(19) United States
(12) Patent Application Publication
(10) Pub. No.: US 2011/0212053 A1
(43) Pub. Date: Sep. 1, 2011

(54) PHOSPHATIDYLINOSITOL 3 KINASE INHIBITORS

(76) Inventors: Dapeng QIAN, Briarcliff Manor, NY (US); Amy Qi Han, Hockessin, DE (US); Mark Hamilton, Hopewell Junction (GB); Eric Wang, San Diego, CA (US)

(21) Appl. No.: 12/737,215

(22) PCT Filed: Jun. 19, 2009
(86) PCT No.: PCT/US09/47970

§ 371 Date: May 3, 2011

Related U.S. Application Data
(60) Provisional application No. 61/073,915, filed on Jun. 19, 2008.

(51) Int. Cl. A61K 38/20; C07D 471/04; C12N 5/09; C07D 498/04; C07D 513/04; A61K 31/437; A61K 31/506; A61K 31/496; A61K 31/501; A61K 31/5377; A61P 35/00; A61P 3/00; A61P 29/00; A61P 37/08; A61P 9/00; A61P 35/02; A61P 19/02; A61P 17/06; A61P 25/00; A61P 11/00; A61P 11/06; A61P 9/10; A61P 9/04; A61P 3/10; A61P 3/04; A61P 37/02; A61P 37/06; A61P 7/00; A61K 38/19; A61K 33/24; A61K 38/50; A61K 38/43; A61K 31/675; A61K 31/573

(52) U.S. Cl. 424/85.2; 546/82; 435/375; 544/333; 544/361; 544/238; 544/126; 544/80; 546/83; 514/293; 514/256; 514/253.03; 514/252.04; 514/232.8; 514/232.5; 424/85.1; 424/649; 424/94.6; 424/94.1; 514/110; 514/180

(57) ABSTRACT

Provided are compounds according to Formula (I):

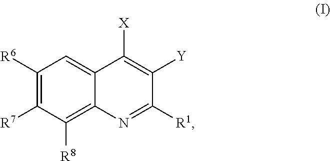

or stereoisomer, prodrug, polymorph, or pharmaceutically acceptable salt forms thereof, wherein X, Y, R¹, R⁶, R⁷, and R⁸ are as defined, and which compounds are effective inhibitors of PI3-kinase and/or other medically and clinically relevant kinases. Also provided are pharmaceutical compositions and methods of using the compounds and compositions as PI3-kinase and kinase inhibitors. More particularly, the compounds of the invention provide treatments and therapeutics for human diseases regulated by, or associated with, the activity of, PI3-kinases and/or protein kinases, or mutant or variant forms thereof.

This application claims benefit of U.S. patent application No. 61/073,915, filed Jun. 19, 2008, the contents of which are incorporated herein in their entirety.

The references cited in this Specification, and their references, are incorporated by reference herein in their entirety where appropriate to more fully describe the state of the art to which this invention relates.

### FIELD OF THE INVENTION

The invention relates to quinoline based compounds, in particular, to small molecules, their stereoisomers, and salts or prodrugs thereof, as inhibitors of phosphatidylinositol 3-kinase (PI3-kinase or PI3K). The invention further relates to the preparation of the described PI3K inhibitor compounds and their use in compositions and as pharmaceuticals for the treatment of various diseases, conditions and disorders.

### BACKGROUND

PI3K comprises a family of lipid kinases that catalyze the phosphorylation of the 3′-OH position of the inositol ring of the glycerol phospholipid, phosphatidylinositol (PI) to produce phosphatidylinositol 3-phosphate (PIP, PI(3)P). (IUPAC-IUB Commission on Biochemical Nomenclature (CBN). Nomenclature of cyclitols. Recommendations 1973. Biochem. J. 153, 23-31 (1976)). PI3K activity yields mono and polyphosphorylated products depending on the phosphorylation state of the substrate, i.e., PI(3)P, PI 3,4-bisphosphate (PI(3,4)P₂, PIP₂) and PI 3,4,5-trisphosphate (PI(3,4,5)P₃, PIP₃). Of these substrates and products, PI(3,4)P₂ and PI(3,4,5)P₃ play a role as recruitment sites for various intracellular signalling proteins which form signalling complexes for the relay of extracellular signalling events to the cytoplasmic face of the cell membrane. PI(3,4,5)P₃ is an especially important signal transduction molecule that has been implicated in many normal physiologic and pathophysiologic processes.

The PI3K family consists of four distinct classes defined by structural and functional characteristics and includes both lipid kinases (Classes I-III) and protein kinases (Class IV). The most fully characterized class of the lipid kinases are the Class I-PI3Ks. Class I comprises three Class Ia isoforms (PI3Kα, PI3Kβ and PI3Kδ) that contain p110 catalytic subunits (p110α, p110β, and p110δ) complexed with a regulatory subunit (p85 or p55). There is a single Class IB PI3Kγ isoform containing a p110γ catalytic subunit complexed with a regulatory p101 subunit. All catalytic p110 subunits share sequence homology and structural similarity including a C2 membrane targeting domain, Ras binding domain, as well as a catalytic kinase domain. In vitro, all PI3Ks can phosphorylate PI, PI(4)P and PI(4,5)P₂to PI(3)P, PI(3,4)P₂ and PI(3,4,5)P₃, respectively. In vivo, only PI(4,5)P₂ is a substrate for PI3Ks. Class Ia PI3Ks are activated through tyrosine kinase signalling and are involved in cell growth, proliferation and survival. PI3Kα and PI3Kβ have been implicated in tumorigenesis in a number of human cancers of various types.

Studies in Drosophila and animal models have indicated that PI3K has a central role in normal development, defining the number and size of cells in tissues. Dysfunction of this pathway leads to growth anomalies and has been established to play a key role in the pathogenesis of Cowden syndrome and tuberous sclerosis among other diseases, pathologies and conditions.

PI3K activity coordinates upstream growth factors with the downstream cellular signals necessary for normal homeostasis, including and cell growth and cell survival. Deregulated or unregulated growth is a hallmark of cancer and the targeting of this biological event is the therapeutic basis of modern anti-cancer strategies including the administration of radiotherapy, chemotherapy, immunological and small molecule agents. More recently, the discovery of nonrandom somatic mutations of the gene encoding PI3Kα in many human tumors suggests an oncogenic role for the mutated enzyme.

PI3Kα appears to be highly relevant in human cancers and malignancies. PI3Kα is overexpressed in human cancers, and activating mutations in the catalytic p110α gene have been identified in both human cancers and tumor cell lines. Accordingly, these activating mutations are suspected to confer a growth advantage. Diverse in vitro observations support this conclusion. For example, human mammary epithelial cells expressing mutated PI3Kα are resistant to cell cycle arrest and apoptosis when exposed to low serum concentrations. Furthermore, the expression of mutated PI3Kα also increases resistance to cytotoxic drugs, and promotes anchorage-independent cell growth in vitro, as well as the growth of tumors in animals.

Class1 PI3K subclasses appear to partition between growth factor receptor tyrosine kinases such as EGFR, and G-protein coupled receptors (GPCRs). Upon ligand stimulation, the Class Ia PI3Kα, β and δ proteins couple to, and are activated by, receptor tyrosine kinases, whereas the PI3Kγ Class Ib enzyme is activated by its association with GPCR βγ subunits released upon GPCR activation. Stimulation of PI3K activity results in the activation of the downstream kinase AKT, a key mediator of PI3K signal transduction and function. The application of inhibitors of PI3K (e.g. wortmannin, LY294002) and of signaling downstream of PI3K (e.g. rapamycin (mTOR)) have helped to define the critical role of the PI3K pathway in relating and integrating extracellular signals to the nuclear events required for promoting cell growth and survival.

An important regulator of PI3K-dependent growth is the tumor suppressor PTEN (phosphatase and tensin homolog). PTEN functions to antagonize PI3-kinase signaling by specifically dephosphorylating 3-OH phosphorylated phosphatidylinositols. In cells, PTEN regulates PI3K signaling by hydrolyzing PI(3,4,5)P₃ to PI(4,5)P₂, and consequently downregulates the signals that control both cell growth and survival. When PTEN activity is removed, the PI3-kinase pathway proceeds unabated. PTEN-inactivating mutations and deletions occur with high frequency in human tumors.

There is significant scientific evidence to suggest that mutational activation of PI3K and/or select receptors that signal through PI3K can sensitize human tumor cells to PI3K inhibitors. However several oncogenes, including those that encode kinases, non-kinases, transcription factors, and GTPases, have now been implicated in resistance to PI3K inhibition, e.g. Src (kinase), Ras (GTPase), Cyclin B (non-kinase), and Myc (transcription). Although the PI3K pathway is the most mutated pathway in human cancers, many of these “resistance factors” are also highly prevalent in human cancers, and could potentially play a role in a large subset of patients who may be poor or non-responders to PI3K selective therapies. This is supported by several preclinical studies demonstrating the lack of efficacy of PI3K inhibitors in tumors harboring mutated Ki-Ras. It has been reported that functional redundancy exists between molecules or factors in different pathways that regulate cell growth, survival, protein translation, etc, such that inhibition of the molecules or factors in one pathway can be overcome by the upregulation or substitution of those in another pathway. In addition, preclinical evidence has demonstrated that PI3K-selective inhibitors (i.e. inhibitors of PI3K family members only) are, in general, cytostatic agents, and that cancer cells and tumors regrow after drug removal. There is therefore a need for inhibitors that not only inhibit the PI3K pathway but also additional, complementary or parallel pathways (e.g. Ras-MAPK) or components of those pathways, e.g. MNK1/2, to minimize or eliminate the potential for pathway redundancy and PI3K inhibitor resistance. In addition, there is a need to develop targeted therapies or signal transduction inhibitors, including PI3K inhibitors, that not only block tumor cell proliferation and tumor growth but are able to induce tumor cell death.

As a therapeutic target, PI3K offers a compelling opportunity to discover and develop new and effective therapies for human diseases. The importance of the activity of PI3K, coupled with the susceptibility of this lipid kinase to mutations that may be associated with tumors, various oncogenic processes, general proliferative diseases, and other diseases, contribute to the relevance of PI3K as a significant therapeutic target. (See, for example, recent reviews by Vogt, P., Bader, A. and Kang, S. 2006 Cell Cycle 5, 946-949 and Admei, A., and Hidalgo, M. 2005 J. Clin. Oncology 23, 5386-5403, and Marone et al. 2008 Biochim. Biophys. Acta 1784 159-185). Although some inhibitors of PI3K have been identified, there is a growing need for other potent and selective PI3K inhibitor compounds, especially those that can target other molecules, particularly clinically relevant molecules that are involved in cell growth, proliferation and survival. Such new inhibitors are valuable, beneficial and advantageous as therapeutic and/or prophylactic treatments for a variety of diseases, disorders and conditions.

### SUMMARY OF THE INVENTION

The present invention provides novel quinoline based compounds, or prodrugs or pharmaceutically acceptable salt forms thereof, which inhibit phosphatidylinositol 3-kinase (PI3-kinase or PI3K).

The PI3K inhibitor compounds of the invention and pharmaceutically acceptable compositions thereof are useful for treating, ameliorating, reducing the severity of, or eliminating a variety of diseases, disorders and conditions, including cancer, tumors, autoimmune diseases, inflammatory diseases, allergic diseases, cardiovascular diseases, diabetes, asthma and organ transplantation rejection in a subject, including human patients, in need thereof.

Compounds of the invention and pharmaceutically acceptable compositions thereof further inhibit other medically and clinically relevant kinases, e.g., protein kinases, such as those involved in, or associated with, various tumors, cancers, neoplasms, and malignancies, gastrointestinal diseases, diseases and disorders of metabolism, inflammatory diseases, autoimmune diseases, and allergic and cardiovascular diseases. Medically and clinically relevant protein kinases targeted by one or more of the compounds of the invention include, but are not limited to, ABL1, ABL2, ALK4, ARKS, AUR A, AXL, BLK, BMX, BRK, BTK, CAMKK2, CDK1, CDK2, CDK3, CDK5, CDK7, CK1δ, CK1ε, CK2α, CK2α2, CLK1, CLK2, CLK3, CLK4, c-MER, c-Src, DYRK1A, DYRK1B, DYRK2, DYRK3, EGFR, EPHA7, FER, FGR, FLT3, FLT4, FMS, FYN, GCK, GSK3α, GSK3β, HCK, HGK, HIPK2, HIPK3, HIPK4, IRAK1, IRAK4, ITK, KDR/VEGFR2, KIT, LCK, LOK, LYN, MELK, MLCK2, MLK1, MNK1, MNK2, MST1, MST2, mTOR, MUSK, NEK1, NEK3, PDGFRα, PDGFRβ, PIM-1, PKCδ (delta), PKCμ (mu), PKCν (nu), PKD2, RET, RIPK2, ROS, RSK1, RSK2, RSK3, RSK4, STK33, TAK1, TAOK1, TAOK3, TRKA, TRKB, TRKC, TTK, TXK, TYK2, YES, ZAK, and or ZAP70 kinases, or mutant, mutationally activated, or variant forms thereof.

The invention provides isolated compounds having at least about 75% purity, at least about 80% purity, at least about 85% purity, at least about 90% purity, at least about 95% purity, at least about 98% purity, at least about 99.5% purity, or at least about 99.8% purity.

The invention provides novel, isolated compounds in a crystal form. The invention provides methods of synthesizing or producing the compounds as described herein.

The present invention provides compositions and pharmaceutical compositions including a pharmaceutically acceptable excipient, carrier, or vehicle, and a therapeutically effective amount of at least one of the compounds of the present invention or a pharmaceutically acceptable salt form thereof.

The present invention provides the compounds of the invention in pharmaceutical compositions in the form of tablets, granules, powders, or capsules for different routes of delivery or administration, such as sublingual, peroral, rectal, or parenteral, including transdermal patch, intravenous, intramuscular, or subcutaneous injection. In one aspect, the invention provides pharmaceutical compositions which are enterically coated.

The present invention provides pharmaceutical compositions wherein the composition is in a controlled release or sustained release formulation, a solution, a topical formulation, lyophilized, a suppository, in an inhaler, a prefilled syringe or a nasal spray device.

The present invention further provides a method of treating PI3K activity related diseases and disorders in a subject in need thereof, comprising administering to a subject a therapeutically effective amount of at least one of the compounds of the invention, or a pharmaceutically acceptable salt form thereof, to treat the PI3-kinase activity related disease or disorder.

The present invention provides a method of treating or targeting PI3K or PI3K-dependent or related signaling pathways, comprising administering to a subject in need of such treatment a therapeutically effective amount of at least one of the compounds of the present invention or a pharmaceutically acceptable salt form thereof. In an embodiment, the compound is an inhibitor of PI3Kα.

The present invention also provides a method for inhibiting or blocking PI3K, or a PI3K-dependent pathway, in the treatment or therapy of cancer, oncogenesis, neoplasms, tumors, or diseases and conditions associated with abnormal PI3K activity. More specifically, embodiments of the present invention provide novel compounds which are useful as inhibitors of PI3K or pharmaceutically acceptable salts thereof, including inhibiting the enzyme activity of converting phosphatidylinositol to phosphatidylinositol 3-phosphate, phosphatidylinositol 4-phosphate to phosphatidylinositol 3,4-bisphosphate and phosphatidinylositol 4,5-bisphosphate to phosphatidinylositol 3,4,5-triphosphate. In an embodiment, the compound is an inhibitor of PI3Kα.

The present invention further provides the described PI3K inhibitor compounds as inhibitors of PI3Kα or the p110α form of PI3K. The invention provides the described PI3K inhibitor compounds as inhibitors of PI3Kβ, PI3Kγ, or PI3Kδ, or the p110β, p110γ (p120γ), or p110δ isoforms of PI3K, as well as different mutant or variant forms thereof, e.g., without limitation, p110α (E542K), p110α (E545K), or p110α (H1047R).

The invention further provides PI3K inhibitor compounds that are potent and selective inhibitors of other kinase activities, such as protein kinases. The PI3K inhibitor compounds of the invention are newly discovered inhibitors of medically and clinically relevant protein kinases, such as those involved in various cancers, tumors, or neoplasms, e.g., breast cancer, hematopoietic cell cancers, lymphocytic cancers, colon cancer, prostate cancer, neural or neuronal cell cancers, brain cancer, glioblastomas, renal cancer, colorectal cancer, pancreatic cancer, non-small cell lung carcinoma (NSCLC), acute lymphoblastic leukemia (ALL); agammaglobulinaemia; gastrointestinal stromal tumors (GIST), bladder cancer, prostate cancer, melanoma, myeloma, acute lymphoblastic leukemia (ALL); agammaglobulinaemia; gastrointestinal stromal tumors (GIST), etc. The invention provides compositions of one or more PI3K inhibitor compounds that also potently and selectively inhibit one or more protein kinases. The invention provides pharmaceutically acceptable compositions containing a therapeutically effective amount of one or more PI3K, e.g., PI3Kα, inhibitor compounds of this invention that inhibit one or more protein kinases, and a pharmaceutically acceptable carrier, excipient or diluent. The invention provides novel compounds that inhibit both PI3K and a protein kinase, which are involved in various diseases and disorders, including, for example, cancers, tumors, inflammatory diseases, allergic diseases, or cardiovascular diseases.

The invention further provides compounds that demonstrate anti-proliferative and apoptotic activity. In an embodiment, the compounds have cytotoxic activity in cells harboring Ras mutations, as demonstrated in Ras mutated cell lines. In an embodiment, compounds of the invention block MNK-eIF4E signaling (protein translation). In an embodiment, compounds of the invention demonstrate cytostatic activity. In an embodiment, compounds of the invention demonstrate both cytostatic and cytotoxic activity. In an embodiment, compounds of the invention demonstrate cytotoxic activity and induce cell death. In an embodiment, compounds of the invention demonstrate pro-apoptotic activity and induce cell death. In an embodiment, compounds of the invention induce caspase activity in tumors harboring mutations that confer resistance to PI3K-selective inhibitors.

The invention provides a method of inducing apoptosis of a tumor or cancer cell, which involves contacting the tumor or cancer cell with a compound as described herein or a composition containing the compound, in an amount effective to induce apoptosis of the tumor or cancer cell. In an embodiment, the tumor or cancer cell is present in a subject and the compound is administered to the subject.

The invention further provides a method of inducing caspase activity in a tumor or cancer cell harboring one or more mutations that confer resistance to a PI3K inhibitor resulting in apoptosis of the tumor or cancer cell which involves contacting the tumor or cancer cell with a compound as described herein or a composition containing the compound, in an amount effective to induce caspase activity in and apoptosis of the tumor or cancer cell. In an embodiment, the tumor or cancer cell harbors at least one mutation in one or more of Ras or Src. In an embodiment, the tumor or cancer cell is present in a subject and the compound is administered to the subject.

In yet another of its aspects, the invention provides a method of inducing caspase activity in a tumor or cancer cell comprising overexpression of a gene or protein that confers resistance to a PI3K inhibitor, and the overexpression results in apoptosis of the tumor or cancer cell which involves contacting the tumor or cancer cell with a compound as described herein or a composition containing the compound, in an amount effective to induce caspase activity in and apoptosis of the tumor or cancer cell. In an embodiment, the tumor or cancer cell overexpresses Myc or cyclin B. In an embodiment, the tumor or cancer cell is present in a subject and the compound is administered to the subject.

The invention further provides a method of inducing cytotoxicity in a tumor or cancer cell by blocking translation of one or more proteins comprising a cellular signal transduction pathway that may lead to aberrant, uncontrolled, or abnormal cell growth and proliferation, in which the method involves contacting the tumor or cancer cell with a compound as described herein or a composition containing the compound, in an amount effective to block the translation of proteins comprising such signal transduction pathway. In an embodiment, the signal transduction pathway does not involve AKT-mTOR or the signaling thereof. In an embodiment, the one or more proteins is MNK, eIF4E, MAPK, RSK, or a combination thereof, such as MKK-eIF4E, or MAPK-RSK.

These and other aspects are provided by the inventive compounds of Formula (I):

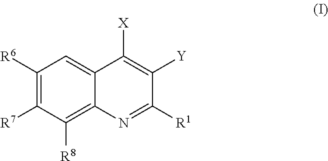

or stereoisomers or pharmaceutically acceptable salt forms thereof, wherein X, Y, R¹, R⁶, R⁷, and R⁸ are as defined below. The compounds of the invention are effective inhibitors of PI3-kinase. In an embodiment, the compounds are effective inhibitors of PI3Kα. In other aspects, the compounds are effective inhibitors of other medicinally and clinically relevant kinases, e.g., protein kinases as described herein.

### DETAILED DESCRIPTION OF THE INVENTION

[1] A first aspect of the invention provides a novel quinoline compound of Formula

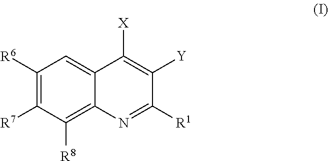

or a stereoisomer, prodrug, or pharmaceutically acceptable salt form, or corresponding polymorph thereof, wherein:
X is NR² or CR², forming a 5 or 6 membered fused heterocycle;
Y is NR³, CR³, S or O, forming a 5 or 6 membered fused heterocycle;
with the proviso that in said 5-membered quinoline fused heterocycle X cannot be NR²;
R¹ is H, OH,
C₁-C₈ alkyl substituted with 0-3 R^{1a},
C₂-C₈ alkenyl substituted with 0-3 R^{1a},
C₂-C₈ alkynyl substituted with 0-3 R^{1a},
C₂-C₈ alkoxy substituted with 0-3 R^{1a},
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, or
5 to 10 membered heterocycle containing 1 to 4 heteroatoms selected from nitrogen, oxygen, and sulfur, wherein said 5 to 10 membered heterocycle is substituted with 0-3 R^{1b};
R^{1a}, at each occurrence, is independently selected from is H, Cl, F, Br, I, CN, NO₂, NR¹²R¹³, OR⁵, SR⁴, C(═O)R₄, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, and
5 to 10 member heterocycle containing 1 to 4 heteroatoms selected from nitrogen, oxygen, and sulphur, wherein said 5 to 10 membered heterocycle is substituted with 0-3 R^{1b};
   with the proviso that said heterocycle is not imidazo
R^{1b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
   with the proviso that R¹ is not

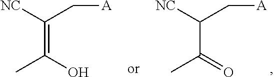

where A is B—(CH₂)ₙ—R^{1c},
B is —CONH—, —SO₂— or —CO—,
n is 1-6, and
R^{1c} is C₁-C₁₄ alkyl,
phenyl,
unsaturated 5-membered heterocycle containing 2 or 3 heteroatoms selected from nitrogen, oxygen, and sulfur,
wherein the phenyl and the unsaturated 5-membered heterocycle are substituted with 0-2 substituents selected independently from halogen, CF₃, hydroxyl, nitro, amino, formylamino, C₁-C₆ alkyl, C₁-C₆ alkoxy, C₂-C₈ alkanoylamino and C₂-C₈ alkanoyloxy;
R² is H, Br,
   C₁-C₈ alkyl substituted with 0-3 R^{2a},
   C₂-C₈ alkenyl substituted with 0-3 R^{2a},
   C₂-C₈ alkynyl substituted with 0-3 R^{2a},
   C₃-C₁₀ carbocycle substituted with 0-3 R^{2b},
   C₁-C₄ sulfonamido substituted with 0-3 R^{2b},
   C₆-C₁₀ aryl substituted with 0-3 R^{2b}, or
   5 to 10 member heterocycle containing 1 to 4 heteroatoms selected from nitrogen, oxygen, and sulfur, wherein said 5 to 10 member heterocycle is substituted with 0-3 R^{2b},
R^{2a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{2b},
   C₁-C₄ sulfonamido substituted with 0-3 R^{2b},
   aryl, arylamine, or allyloxy, at each occurrence substituted with 0-3 R^{2b}, and
   5 to 10 membered heterocycle containing 1 to 4 heteroatoms selected from nitrogen, oxygen, and sulphur, wherein said 5 to 10 membered heterocycle is substituted with 0-3 R^{2b};
R^{2b}, at each occurrence, is independently selected from H, OH, Cl, F, Br, I, CN, NO₂, thiazole, NR¹S(═O)₂CH₃, NR¹₂R¹³, CF₃, acetyl, SCH₃, S(═O)CH₃, S(═O)₂CH₃, H₂N—C(═O)—,
   C₁-C₆ alkyl, C₁-C₄ alkoxy, C₁-C₄ haloalkyl, C₁-C₆ cyanoalkyl,
   C₁-C₄ haloalkoxy, C₁-C₄ cyanoalkoxy, and C₁-C₄ haloalkyl-S—,
   C₁-C₄ alkylhydroxy, C₁-C₄alkylcyano;
R³ is H, O, S,
   C₁-C₈ alkyl substituted with 0-3 R^{3a},
   C₁-C₈ alkylphenyl substituted with 0-3 R^{3a},
   C₂-C₈ alkenyl substituted with 0-3 R^{3a},
   C₂-C₈ alkynyl substituted with 0-3 R^{3a},
   C₂-C₈ alkoxy substituted with 0-3 R^{3a},
   C₃-C₁₀ carbocycle substituted with 0-3 R^{3b},
   C₁-C₄ sulfonamido substituted with 0-3 R^{3b},
   C₆-C₁₀ aryl substituted with 0-3 R^{3b}, or
   5 to 10 membered heterocycle containing 1 to 4 heteroatoms selected from nitrogen, oxygen, and sulfur, wherein said 5 to 10 membered heterocycle is substituted with 0-3 R^{3b},
R^{3a}, at each occurrence, is independently selected from is H, Cl, F, Br, I, CN, NO₂, NR¹²R¹³, OR⁵, SR⁴, C(═O)R⁴ providing the NR² is not substituted by R^{2a} being C(═O)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{3b},
   C₁-C₄ sulfonamido substituted with 0-3 R^{3b},
   C₆-C₁₀ aryl substituted with 0-3 R^{3b}, and
   5 to 10 membered heterocycle containing 1 to 4 heteroatoms selected from nitrogen, oxygen, and sulphur, wherein said 5 to 10 membered heterocycle is substituted with 0-3 R^{3b};
R^{3b}, at each occurrence, is independently selected from H, OH, Cl, F, Br, I, CN, NO₂, NR¹²R¹³, CF₃, acetyl, SCH₃, S(═O)CH₃, S(═O)₂CH₃, H₂N—C(═O)—, NR¹²R¹³C(═O)—
   C₁-C₆ alkyl, C₁-C₄ alkoxy, C₁-C₄ haloalkyl, C₁-C₆ cyanoalkyl,
   C₁-C₄ haloalkoxy, C₁-C₄ cyanoalkoxy, and C₁-C₄ haloalkyl-S—;
R⁴ is H, phenyl, benzyl, C₁-C₄ alkyl, C₃-C₈ cycloalkyl substituted with 0-3 R^{1b}, or
a 5 to 10 member heterocycle containing 1 to 4 heteroatoms selected from nitrogen, oxygen, and sulphur, wherein said 5 to 10 member heterocycle is substituted with 0-3 R^{1b};
R⁵ is H, phenyl, benzyl, or C₁-C₄ alkyl;
R⁶ is H, R^{6a},
   C₁-C₈ alkyl substituted with 0-3 R^{6a},
   C₂-C₈ alkenyl substituted with 0-3 R^{6a},
   C₂-C₈ alkynyl substituted with 0-3 R^{6a},
   C₃-C₁₀ carbocycle substituted with 0-3 R^{6b}, or
   C₁-C₄ sulfonamido substituted with 0-3 R^{6b},
   aryl, arylamine, or alkyloxy, at each occurrence substituted with 0-3 R^{6b}, or
   5 to 13 membered heterocycle containing 1 to 3 fused rings containing 1 to 4 heteroatoms selected from nitrogen, CF, oxygen, and sulfur, wherein said 5 to 10 membered heterocycle is substituted with 0-3 R^{6b},
except where R₆ is in the form of —C(R^{6c})(R^{6d})—NH—CH(R^{6e})(R^{6f}),
   wherein R^{6c} and R^{6d} are independently H, C₁₋₄ haloalkyl or C₁₋₈ alkyl, and
   R^{6e} is a C₁₋₈alkyl or C₁₋₈ alkyl or C₁₋₄ haloalkyl, and
   R^{6f} is phenyl, benzyl, naphthyl or saturated or unsaturated 5- or 6-membered heterocycle containing 1, 2 or 3 atoms selected from nitrogen, oxygen and sulphur with no more than two substituent atoms selected from oxygen and sulphur, and
   wherein said phenyl, benzyl or heterocycle contain 0-3 substituents selected from C₁₋₆ alkyl, C₁₋₄ haloalkyl, —OC₁₋₆ alkyl, halogen, cyano and nitro;
R^{6a}, at each occurrence, is independently selected from is H, OH, Cl, F, Br, I, CN, NO₂, NR¹²R¹³, OR⁵, SR⁴, C(═O)R⁴, —C(═O)NR¹²R¹³, NR¹⁴R¹⁵, S(═O)R⁶, S(═O)₂R¹⁵,
   C(═O)NH₂, —C(═O)phenyl, —Ophenyl, —Opyridyl, phenoxy, CₙF₂ₙ₊₁ (n=1-3),
   C₁-C₆ alkyl, C₁-C₄ alkylphenyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, and
   5 to 10 membered heterocycle containing 1 to 4 heteroatoms selected from nitrogen, oxygen, and sulphur, wherein said 5 to 10 membered heterocycle is substituted with 0-3 R^{1b};
R^{6b}, at each occurrence, is independently selected from H, OH, Cl, F, Br, I, CN, NO₂, NR¹²R¹³, N(C₁-C₄)alkylphenyl, C(═O)NR¹²R¹³, CF₃, acetyl, SCH₃, S(═O)CH₃, S(═O)₂CH₃, S(═O)₂phenyl, C(═O)NR¹²R¹³,
   C₁-C₆ alkyl, C₁-C₄ alkoxy, C₁-C₄ haloalkyl,
   C₁-C₄ haloalkoxy, and C₁-C₄ haloalkyl-S—;
R⁷ is C₁-C₄ alkyl, C₂-C₄ alkenyl, or C₃-C₄ alkynyl or R^{8a};
R⁸ is H, R^{8a},
   C₁-C₈ alkyl substituted with 0-3 R^{8a},
   C₂-C₈ alkenyl substituted with 0-3 R^{8a},
   C₂-C₈ alkynyl substituted with 0-3 R^{8a}
   C₃-C₁₀ carbocycle substituted with 0-3 R^{8b},
   C₁-C₄ sulfonamido substituted with 0-3 R^{8b},
   C₆-C₁₀ aryl substituted with 0-3 R^{8b}, or
   5 to 10 membered heterocycle containing 1 to 4 heteroatoms selected from nitrogen, oxygen, and sulfur, wherein said 5 to 10 membered heterocycle is substituted with 0-3 R^{8b},
R^{8a}, at each occurrence, is independently selected from is H, Cl, F, Br, I, CN, NO₂, NR¹²R¹³, OR₅, SR⁴, C(═O)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{8b},
   C₁-C₄ sulfonamido substituted with 0-3 R^{8b},
   C₆-C₁₀ aryl substituted with 0-3 R^{8b}, and
   5 to 10 membered heterocycle containing 1 to 4 heteroatoms selected from nitrogen, oxygen, and sulphur, wherein said 5 to 10 membered heterocycle is substituted with 0-3 R^{8b};
R^{8b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R¹², at each occurrence, is independently selected from H, C₁-C₆ alkyl, benzyl, phenethyl, (C₁-C₆ alkyl)-C(═O)—, (C₁-C₆ alkyl)-OC(═O)—, (C₁-C₆ alkyl)-S(═O)₂—, and piperidinyl C(═O)—;
R¹³, at each occurrence, is independently selected from
   H, OH, C₁-C₆ alkyl, benzyl, phenethyl,
   (C₁-C₆ alkyl)-C(═O)—, (C₁-C₆)cycloalkyl)C(═O)NH and (C₁-C₆ alkyl)-S(═O)₂—;
alternatively, R¹² and R¹³ together with the nitrogen to which they are attached, may combine to form a 4-7 member ring substituted with 0-3 R^{1b} wherein said 4-7 member ring optionally contains an additional heteroatom selected from O and NH;
R¹⁴, at each occurrence, is independently selected from H, C₁-C₆ alkyl, benzyl, phenethyl, (C₁-C₆ alkyl)-C(═O)—, and (C₁-C₆ alkyl)-S(═O)₂—;
R¹⁵, at each occurrence, is independently selected from
   H, OH, C₁-C₆ alkyl, phenyl, benzyl, phenethyl, (C₁-C₆ alkyl)-OC(═O)—,
   (C₁-C₆ alkyl)-C(═O)—, and (C₁-C₆ alkyl)-S(═O)₂—; and
alternatively, R¹⁴ and R¹⁵, may combine together with the nitrogen to
   which they are attached, to form a 4 to 7 membered ring substituted with 0-3 R^{6a},
      wherein said 4 to 7 membered ring optionally contains
      an heteroatom selected from O and NH.
[2] Another embodiment of the invention provides a compound of Formula (II):

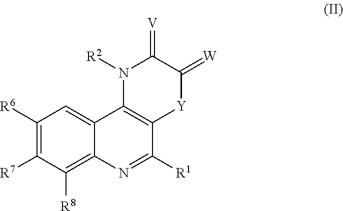

or a stereoisomer, prodrug, or pharmaceutically acceptable salt form, or corresponding polymorph thereof, wherein:
Y is NR³, CR³, or O,
V and W are independently H or O
   with the proviso that W is H when V is O; and when W and V are H, Y is not NR³,
R₁ is H, OH,
   C₁-C₈ alkyl substituted with 0-3 R^{1a},
   C₂-C₈ alkenyl substituted with 0-3 R^{1a},
   C₂-C₈ alkynyl substituted with 0-3 R^{1a},
   C₂-C₈ alkoxy substituted with 0-3 R^{1a},
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, or
   5 to 10 membered heterocycle containing 1 to 4 heteroatoms selected from nitrogen, oxygen, and sulfur, wherein said 5 to 10 membered heterocycle is substituted with 0-3 R^{1b},
R^{1a}, at each occurrence, is independently selected from is H, Cl, F, Br, I, CN, NO2, NR¹²R¹³, OR⁵, SR⁴, C(═O)R₄, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, and
   5 to 10 membered heterocycle containing 1 to 4 heteroatoms selected from nitrogen, oxygen, and sulphur, wherein said 5 to 10 membered heterocycle is substituted with 0-3 R^{1b};
R^{1b}, at each occurrence, is independently selected from H, OH, Cl, F, Br, I, CN, NO₂, NR¹²R¹³, CF₃, acetyl, phenyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R² is H,
   C₁-C₈ alkyl substituted with 0-3 R^{2a},
   C₂-C₈ alkenyl substituted with 0-3 R^{2a},
   C₂-C₈ alkynyl substituted with 0-3 R^{2a},
   C₃-C₁₀ carbocycle substituted with 0-3 R^{2b},
   C₁-C₆ sulfonamido substituted with 0-3 R^{2b},
   C₆-C₁₀ aryl substituted with 0-3 R^{2b}, or
   5 to 10 membered heterocycle containing 1 to 4 heteroatoms selected from nitrogen, oxygen, and sulfur, wherein said 5 to 10 membered heterocycle is substituted with 0-3 R^{2b},
R^{2a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{2b},
   C₁-C₄ sulfonamido substituted with 0-3 R^{2b},
   C₆-C₁₀ aryl substituted with 0-3 R^{2b}, and
   5 to 10 membered heterocycle containing 1 to 4 heteroatoms selected from nitrogen, oxygen, and sulphur, wherein said 5 to 10 membered heterocycle is substituted with 0-3 R^{2b};
R^{2b}, at each occurrence, is independently selected from H, OH, Cl, F, Br, I, CN, NO₂, thiazole, NR¹²R¹³, CF₃, acetyl, SCH₃, S(═O)CH₃, S(═O)₂CH₃, H₂N—C(═O)—,
   C₁-C₆ alkyl, C₁-C₄ alkoxy, C₁-C₄ haloalkyl, C₁-C₆ cyanoalkyl,
   C₁-C₄ haloalkoxy, C₁-C₄ cyanoalkoxy, and C₁-C₄ haloalkyl-S—;
R³ is H,
   C₁-C₈ alkyl substituted with 0-3 R^{3a},
   C₂-C₈ alkenyl substituted with 0-3 R^{3a},
   C₂-C₈ alkynyl substituted with 0-3 R^{3a}
   C₃-C₁₀ carbocycle substituted with 0-3 R^{3b},
   C₁-C₄ sulfonamido substituted with 0-3 R^{3b},
   C₆-C₁₀ aryl substituted with 0-3 R^{3b}, or
   5 to 10 membered heterocycle containing 1 to 4 heteroatoms selected from nitrogen, oxygen, and sulfur, wherein said 5 to 10 membered heterocycle is substituted with 0-3 R^{3b},
R^{3a}, at each occurrence, is independently selected from is H, Cl, F, Br, I, CN, NO₂, NR¹²R¹³, OR⁵, SR⁴, C(═)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{3b},
   C₁-C₄ sulfonamido substituted with 0-3 R^{3b},
   C₆-C₁₀ aryl substituted with 0-3 R^{3b}, and
   5 to 10 membered heterocycle containing 1 to 4 heteroatoms selected from nitrogen, oxygen, and sulphur, wherein said 5 to 10 membered heterocycle is substituted with 0-3 R^{3b};
R^{3b}, at each occurrence, is independently selected from H, OH, Cl, F, Br, I, CN, NO₂, NR¹²R¹³, CF₃, acetyl, SCH₃, S(═O)CH₃, S(═O)₂CH₃, H₂N—C(═O)—,
   C₁-C₆ alkyl, C₁-C₄ alkoxy, C₁-C₄ haloalkyl, C₁-C₆ cyanoalkyl,
   C₁-C₄ haloalkoxy, C₁-C₄ cyanoalkoxy, and C₁-C₄ haloalkyl-S—;
R⁴ is H, phenyl, benzyl, C₁-C₄ alkyl, C₃-C₈ cycloalkyl substituted with 0-3 R^{1b}, or
a 5 to 10 membered heterocycle containing 1 to 4 heteroatoms selected from nitrogen, oxygen, and sulphur, wherein said 5 to 10 membered heterocycle is substituted with 0-3 R^{1b};
R⁵ is H, phenyl, benzyl, or C₁-C₄ alkyl;
R⁶ is H,
   C₁-C₈ alkyl substituted with 0-3 R^{6a},
   C₂-C₈ alkenyl substituted with 0-3 R^{6a},
   C₂-C₈ alkynyl substituted with 0-3 R^{6a},
   C₃-C₁₀ carbocycle substituted with 0-3 R^{6b},
   C₁-C₄ sulfonamido substituted with 0-3 R^{6b},
   C₆-C₁₀aryl substituted with 0-3 R^{6a}; arylamine substituted with 0-3 R^{6a},
   C₁-C₆ alkyloxy substituted with 0-3 R^{6a}, or
   5 to 10 membered heterocycle containing 1 to 4 heteroatoms selected from nitrogen, oxygen, and sulfur, wherein said 5 to 10 membered heterocycle is substituted with 0-3 R^{6b},
R^{6a}, at each occurrence, is independently selected from is H, Cl, F, Br, I, CN, NO₂, NR¹²R¹³, OR⁵, SR⁴, C(═O)R⁴, NR₅R⁶, S(═O)R⁶, S(═O)₂R⁶,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, and
   5 to 10 membered heterocycle containing 1 to 4 heteroatoms selected from nitrogen, oxygen, and sulphur, wherein said 5 to 10 membered heterocycle is substituted with 0-3 R^{1b};
R^{6b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R⁷ is C₁-C₄ alkyl, C₂-C₄ alkenyl, or C₃-C₄ alkynyl;
R⁸ is H,
   C₁-C₈ alkyl substituted with 0-3 R^{8a},
   C₂-C₈ alkenyl substituted with 0-3 R^{8a},
   C₂-C₈ alkynyl substituted with 0-3 R^{8a}
   C₃-C₁₀ carbocycle substituted with 0-3 R^{8b},
   C₁-C₄ sulfonamido substituted with 0-3 R^{8b},
   C₆-C₁₀ aryl substituted with 0-3 R^{8b}, or
   5 to 10 membered heterocycle containing 1 to 4 heteroatoms selected from nitrogen, oxygen, and sulfur, wherein said 5 to 10 membered heterocycle is substituted with 0-3 R^{8b},
R^{8a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R⁴,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{8b},
   C₁-C₄ sulfonamido substituted with 0-3 R^{8b},
   C₆-C₁₀ aryl substituted with 0-3 R^{8b}, and
   5 to 10 membered heterocycle containing 1 to 4 heteroatoms selected from nitrogen, oxygen, and sulphur, wherein said 5 to 10 membered heterocycle is substituted with 0-3 R^{8b};
R^{8b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R¹², at each occurrence, is independently selected from H, C₁-C₆ alkyl, benzyl, phenethyl, (C₁-C₆ alkyl)-C(═O)—, (C₁-C₆ alkyl)-OC(═O)—, (C₁-C₆ alkyl)-S(═O)₂—, and piperdinyl C(═O)—;
R¹³, at each occurrence, is independently selected from
   H, OH, C₁-C₆ alkyl, benzyl, phenethyl,
   (C₁-C₆ alkyl)-C(═O)—, and (C₁-C₆ alkyl)-S(═O)₂—;
alternatively, R¹² and R¹³ together with the nitrogen to which they are attached, may combine to form a 4-7 member ring wherein said 4-7 member ring optionally contains an additional heteroatom selected from O and NH;
R¹⁴, at each occurrence, is independently selected from H, C₁-C₆ alkyl, benzyl, phenethyl, (C₁-C₆ alkyl)-C(═O)—, and (C₁-C₆ alkyl)-S(═O)₂—;
R¹⁵, at each occurrence, is independently selected from
   H, OH, C₁-C₆ alkyl, benzyl, phenethyl, (C₁-C₆ alkyl)-OC(═O)—,
   (C₁-C₆ alkyl)-C(═O)—, and (C₁-C₆ alkyl)-S(═O)₂—; and
alternatively, R¹⁴ and R¹⁵, may combine together with the nitrogen to
   which they are attached, to form a 4 to 7 membered ring,
      wherein said 4 to 7 membered ring optionally contains
      a heteroatom selected from O and NH.
[3] Another embodiment of the invention provides a compound of the Formula (III)

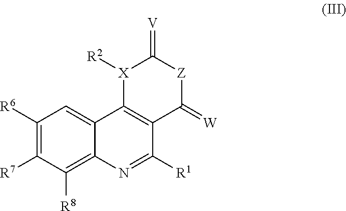

or a stereoisomer, prodrug, or pharmaceutically acceptable salt form, or corresponding polymorph thereof, wherein:
X is N or C;
V and W are independently a single H or O,
W is a single H when V is O;
Z is O, CR₃ or NR₃;
R¹ is H, OH,
   C₁-C₈ alkyl substituted with 0-3 R^{1a},
   C₂-C₈ alkenyl substituted with 0-3 R^{1a},
   C₂-C₈ alkynyl substituted with 0-3 R^{1a},
   C₂-C₈ alkoxy substituted with 0-3 R^{1a},
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, or
   5 to 10 membered heterocycle containing 1 to 4 heteroatoms selected from nitrogen, oxygen, and sulfur, wherein said 5 to 10 membered heterocycle is substituted with 0-3 R^{1b},
R^{1a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, and
   5 to 10 membered heterocycle containing 1 to 4 heteroatoms selected from nitrogen, oxygen, and sulphur, wherein said 5 to 10 membered heterocycle is substituted with 0-3 R^{1b};
R^{1b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R² is H,
   C₁-C₈ alkyl substituted with 0-3 R^{2a},
   C₂-C₈ alkenyl substituted with 0-3 R^{2a},
   C₂-C₈ alkynyl substituted with 0-3 R^{2a}
   C₃-C₁₀ carbocycle substituted with 0-3 R^{2b},
   C₁-C₄ sulfonamido substituted with 0-3 R^{2b},
   C₆-C₁₀ aryl substituted with 0-3 R^{2b}, or
   5 to 10 membered heterocycle containing 1 to 4 heteroatoms selected from nitrogen, oxygen, and sulfur, wherein said 5 to 10 membered heterocycle is substituted with 0-3 R^{2b},
R^{2a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{2b},
   C₁-C₄ sulfonamido substituted with 0-3 R^{2b},
   C₆-C₁₀ aryl substituted with 0-3 R^{2b}, and
   5 to 10 membered heterocycle containing 1 to 4 heteroatoms selected from nitrogen, oxygen, and sulphur, wherein said 5 to 10 membered heterocycle is substituted with 0-3 R^{2b};
R^{2b}, at each occurrence, is independently selected from H, OH, Cl, F, Br, I, CN, NO₂, thiazole, NR¹²R¹³, CF₃, acetyl, SCH₃, S(═O)CH₃, S(═O)₂CH₃, H₂N—C(═O)—,
   C₁-C₆ alkyl, C₁-C₄ alkoxy, C₁-C₄ haloalkyl, C₁-C₆ cyanoalkyl,
   C₁-C₄ haloalkoxy, C₁-C₄ cyanoalkoxy, and C₁-C₄ haloalkyl-S—;
R³ is H, O,
   C₁-C₈ alkyl substituted with 0-3 R^{3a},
   C₂-C₈ alkenyl substituted with 0-3 R^{3a},
   C₂-C₈ alkynyl substituted with 0-3 R^{3a}
   C₃-C₁₀ carbocycle substituted with 0-3 R^{3b},
   C₁-C₄ sulfonamido substituted with 0-3 R^{3b},
   C₆-C₁₀ aryl substituted with 0-3 R^{3b}, or
   5 to 10 membered heterocycle containing 1 to 4 heteroatoms selected from nitrogen, oxygen, and sulfur, wherein said 5 to 10 membered heterocycle is substituted with 0-3 R^{3b},
R^{3a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{3b},
   C₁-C₄ sulfonamido substituted with 0-3 R^{3b},
   C₆-C₁₀ aryl substituted with 0-3 R^{3b}, and
   5 to 10 membered heterocycle containing 1 to 4 heteroatoms selected from nitrogen, oxygen, and sulphur, wherein said 5 to 10 membered heterocycle is substituted with 0-3 R^{3b};
R^{3b}, at each occurrence, is independently selected from H, OH, Cl, F, Br, I, CN, NO₂, NR¹²R¹³, CF₃, acetyl, SCH₃, S(═O)CH₃, S(═O)₂CH₃, H₂N—C(═O)—,
   C₁-C₆ alkyl, C₁-C₄ alkoxy, C₁-C₄ haloalkyl, C₁-C₆ cyanoalkyl,
   C₁-C₄ haloalkoxy, C₁-C₄ cyanoalkoxy, and C₁-C₄ haloalkyl-S—;
R⁴ is H, phenyl, benzyl, C₁-C₄ alkyl, C₃-C₈ cycloalkyl substituted with 0-3 R^{1b}, or
a 5 to 10 membered heterocycle containing 1 to 4 heteroatoms selected from nitrogen, oxygen, and sulphur, wherein said 5 to 10 membered heterocycle is substituted with 0-3 R^{1b};
R⁵ is H, phenyl, benzyl, or C₁-C₄ alkyl;
R⁶ is H,
   C₁-C₈ alkyl substituted with 0-3 R^{6a},
   C₂-C₈ alkenyl substituted with 0-3 R^{6a},
   C₂-C₈ alkynyl substituted with 0-3 R^{6a}
   C₃-C₁₀ carbocycle substituted with 0-3 R^{6b},
   C₁-C₄ sulfonamido substituted with 0-3 R^{6b},
   C₆-C₁₀ aryl substituted with 0-3 R^{6a}; arylamine substituted with 0-3 R^{6a},
   C₁-C₆ alkyloxy substituted with 0-3 R^{6a}, or
   5 to 10 membered heterocycle containing 1 to 4 heteroatoms selected from nitrogen, oxygen, and sulfur, wherein said 5 to 10 membered heterocycle is substituted with 0-3 R^{6b},
R^{6a}, at each occurrence, is independently selected from is H, Cl, F, Br, I, CN, NO₂, NR¹²R¹³, OR₅, SR₄, C(═O)R⁴, NR⁵R⁶, S(═O)R⁶, S(═O)₂R⁶,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, and
   5 to 10 membered heterocycle containing 1 to 4 heteroatoms selected from nitrogen, oxygen, and sulphur, wherein said 5 to 10 membered heterocycle is substituted with 0-3 R^{1b};
R^{6b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R⁷ is C₁-C₄ alkyl, C₂-C₄ alkenyl, or C₃-C₄ alkynyl;
R⁸ is H,
   C₁-C₈ alkyl substituted with 0-3R^{8a},
   C₂-C₈ alkenyl substituted with 0-3 R^{8a},
   C₂-C₈ alkynyl substituted with 0-3 R^{8a},
   C₃-C₁₀ carbocycle substituted with 0-3 R^{8b},
   C₁-C₄ sulfonamido substituted with 0-3 R^{8b},
   C₆-C₁₀ aryl substituted with 0-3 R^{8b}, or
   5 to 10 membered heterocycle containing 1 to 4 heteroatoms selected from nitrogen, oxygen, and sulfur, wherein said 5 to 10 membered heterocycle is substituted with 0-3 R^{8b},
R^{8a}, at each occurrence, is independently selected from is H, Cl, F, Br, I, CN, NO₂, NR¹²R¹³, OR⁵, SR⁴, C(═O)R₄, NR¹⁴R¹⁵, S(═O)R⁶, S(═O)₂R⁴,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{8b},
   C₁-C₄ sulfonamido substituted with 0-3 R^{8b},
   C₆-C₁₀ aryl substituted with 0-3 R^{8b}, and
   5 to 10 membered heterocycle containing 1 to 4 heteroatoms selected from nitrogen, oxygen, and sulphur, wherein said 5 to 10 membered heterocycle is substituted with 0-3 R^{8b};
R^{8b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R¹², at each occurrence, is independently selected from H, C₁-C₆ alkyl, benzyl, phenethyl, (C₁-C₆ alkyl)-C(═O)—, (C₁-C₆ alkyl)-OC(═O)—, (C₁-C₆ alkyl)-S(═O)₂—, and piperdinyl C(═O)—;
R¹³, at each occurrence, is independently selected from
   H, OH, C₁-C₆ alkyl, benzyl, phenethyl,
   (C₁-C₆ alkyl)-C(═O)—, and (C₁-C₆ alkyl)-S(═O)₂—;
alternatively, R¹² and R¹³ together with the nitrogen to which they are attached, may combine to form a 4 to 7 member ring wherein said 4 to 7 membered ring optionally contains an additional heteroatom selected from O and NH;
R¹⁴, at each occurrence, is independently selected from H, C₁-C₆ alkyl, benzyl, phenethyl, (C₁-C₆ alkyl)-C(═O)—, and (C₁-C₆ alkyl)-S(═O)₂—;
R¹⁵, at each occurrence, is independently selected from
   H, OH, C₁-C₆ alkyl, benzyl, phenethyl, (C₁-C₆ alkyl)-OC(═O)—,
   (C₁-C₆ alkyl)-C(═O)—, and (C₁-C₆ alkyl)-S(═O)₂—; and
alternatively, R¹⁴ and R¹⁵, may combine together with the nitrogen to
   which they are attached, to form a 4-7 membered ring,
      wherein said 4-7 membered ring optionally contains
      an heteroatom selected from O and NH.
[4] Another embodiment of the invention provides a compound of the Formula (IV)

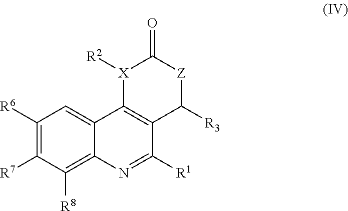

or a stereoisomer, prodrug, or pharmaceutically acceptable salt form, or corresponding polymorph thereof, wherein:
X is N or C;
Z is O, CR³ or NR³ and all other symbols are as described in Formula (III).
[5] Another embodiment of the invention provides a novel compound of Formula (V),

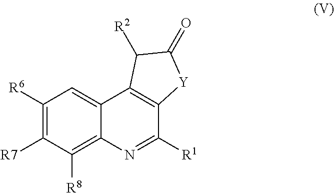

or a stereoisomer, prodrug, pharmaceutically acceptable salt form, or corresponding polymorph thereof,
wherein:
Y is O, CR³ or NR³;
R¹ is H, OH,
   C₁-C₈ alkyl substituted with 0-3 R^{1a},
   C₂-C₈ alkenyl substituted with 0-3 R^{1a},
   C₂-C₈ alkynyl substituted with 0-3 R^{1a},
   C₂-C₈ alkoxy substituted with 0-3 R^{1a},
   C₃-C₁₀ carbocycle substituted with 0-3 R¹_{b},
   C₁-C₄ sulfonamido substituted with 0-3 R^{1b},
   C₆-C₁₀ aryl substituted with 0-3 R^{1b}, or
   5 to 10 membered heterocycle containing 1 to 4 heteroatoms selected from nitrogen, oxygen, and sulfur, wherein said 5 to 10 membered heterocycle is substituted with 0-3 R^{1b},
R^{1a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R_{1b},
   C₁-C₄ sulfonamido substituted with 0-3 R_{1b},
   C₆-C₁₀ aryl substituted with 0-3 R_{1b}, and
   5 to 10 membered heterocycle containing 1 to 4 heteroatoms selected from nitrogen, oxygen, and sulphur, wherein said 5 to 10 membered heterocycle is substituted with 0-3 R_{1b};
R^{1b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R² is H, O,
   C₁-C₈ alkyl substituted with 0-3 R^{2a},
   C₂-C₈ alkenyl substituted with 0-3 R^{2a},
   C₂-C₈ alkynyl substituted with 0-3 R^{2a}
   C₃-C₁₀ carbocycle substituted with 0-3 R^{2b},
   C₁-C₄ sulfonamido substituted with 0-3 R^{2b},
   C₆-C₁₀ aryl substituted with 0-3 R^{2b}, or
   5 to 10 membered heterocycle containing 1 to 4 heteroatoms selected from nitrogen, oxygen, and sulfur, wherein said 5 to 10 membered heterocycle is substituted with 0-3 R^{2b},
R^{2a}, at each occurrence, is independently selected from H, Cl, F, Br, I, CN, NO₂, NR¹²R¹³, OR⁵, SR⁴, C(═O)R⁴, NR¹⁴R¹⁵, S(═O)R₆, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{2b},
   C₁-C₄ sulfonamido substituted with 0-3 R^{2b},
   C₆-C₁₀ aryl substituted with 0-3 R^{2b}, and
   5 to 10 membered heterocycle containing 1 to 4 heteroatoms selected from nitrogen, oxygen, and sulphur, wherein said 5 to 10 membered heterocycle is substituted with 0-3 R^{2b};
R^{2b}, at each occurrence, is independently selected from H, OH, Cl, F, Br, I, CN, NO₂, thiazole, NR¹²R¹³, CF₃, acetyl, SCH₃, S(═O)CH₃, S(═O)₂CH₃, H₂N—C(═O)—,
   C₁-C₆ alkyl, C₁-C₄ alkoxy, C₁-C₄ haloalkyl, C₁-C₆ cyanoalkyl,
   C₁-C₄ haloalkoxy, C₁-C₄ cyanoalkoxy, and C₁-C₄ haloalkyl-S—;
R³ is H,
   C₁-C₈ alkyl substituted with 0-3 R^{3a},
   C₂-C₈ alkenyl substituted with 0-3 R^{3a},
   C₂-C₈ alkynyl substituted with 0-3 R^{3a},
   C₃-C₁₀ carbocycle substituted with 0-3 R^{3a},
   C₁-C₄ sulfonamido substituted with 0-3 R^{3a},
   aryl substituted with 0-3 R^{3a}, or
   5 to 10 membered heterocycle containing 1 to 4 heteroatoms selected from nitrogen, oxygen, and sulfur, wherein said 5 to 10 membered heterocycle is substituted with 0-3 R^{3a},
R^{3a}, at each occurrence, is independently selected from H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{3b},
   C₁-C₄ sulfonamido substituted with 0-3 R^{3b},
   C₆-C₁₀ aryl substituted with 0-3 R^{3b}, and
   5 to 10 membered heterocycle containing 1 to 4 heteroatoms selected from nitrogen, oxygen, and sulphur, wherein said 5 to 10 membered heterocycle is substituted with 0-3 R^{3b};
R^{3b}, at each occurrence, is independently selected from H, OH, Cl, F, Br, I, CN, NO₂, NR₁₂R₁₃, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R⁴ is H, phenyl, benzyl, C₁-C₄ alkyl, C₃-C₈ cycloalkyl substituted with 0-3 R^{1b}, or
a 5 to 10 membered heterocycle containing 1 to 4 heteroatoms selected from nitrogen, oxygen, and sulphur, wherein said 5 to 10 membered heterocycle is substituted with 0-3 R^{1b};
R⁵ is H, phenyl, benzyl, or C₁-C₄ alkyl;
R⁶ is H,
   C₁-C₈ alkyl substituted with 0-3 R^{6a},
   C₂-C₈ alkenyl substituted with 0-3 R^{6a},
   C₂-C₈ alkynyl substituted with 0-3 R^{6a}
   C₃-C₁₀ carbocycle substituted with 0-3 R^{6b},
   C₁-C₄ sulfonamido substituted with 0-3 R₆^{b},
   C₆-C₁₀ aryl substituted with 0-3 R^{6a}; arylamine substituted with 0-3 R^{6a},
   C₁-C₆ alkyloxy substituted with 0-3 R^{6a}, or
   5 to 10 membered heterocycle containing 1 to 4 heteroatoms selected from nitrogen, oxygen, and sulfur, wherein said 5 to 10 membered heterocycle is substituted with 0-3 R^{6b},
R^{6a}, at each occurrence, is independently selected from is H, Cl, F, Br, I, CN, NO₂, NR¹²R¹³, OR⁵, SR⁴, C(═O)R₄, NR⁵R⁶, S(═O)R⁶, S(═O)₂R⁶,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, and
   5 to 10 membered heterocycle containing 1 to 4 heteroatoms selected from nitrogen, oxygen, and sulphur, wherein said 5 to 10 membered heterocycle is substituted with 0-3 R^{1b};
R^{6b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R⁷ is H, C₁-C₄ alkyl, C₂-C₄ alkenyl, or C₃-C₄ alkynyl;
R⁸ is H,
   C₁-C₈ alkyl substituted with 0-3 R^{8a},
   C₂-C₈ alkenyl substituted with 0-3 R^{8a},
   C₂-C₈ alkynyl substituted with 0-3 R^{8a},
   C₃-C₁₀ carbocycle substituted with 0-3 R^{8b},
   C₁-C₄ sulfonamido substituted with 0-3 R^{8b},
   C₆-C₁₀ to aryl substituted with 0-3 R^{8b}, or
   5 to 10 membered heterocycle containing 1 to 4 heteroatoms selected from nitrogen, oxygen, and sulfur, wherein said 5 to 10 membered heterocycle is substituted with 0-3 R^{8b},
R^{8a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R⁴,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{8b},
   C₁-C₄ sulfonamido substituted with 0-3 R^{8b},
   C₆-C₁₀ aryl substituted with 0-3 R^{8b}, and
   5 to 10 membered heterocycle containing 1 to 4 heteroatoms selected from nitrogen, oxygen, and sulphur, wherein said 5 to 10 membered heterocycle is substituted with 0-3 R_{8b};
R^{8b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R¹², at each occurrence, is independently selected from H, C₁-C₆ alkyl, benzyl, phenethyl, (C₁-C₆ alkyl)-C(═O)—, (C₁-C₆ alkyl)-OC(═O)—, (C₁-C₆ alkyl)-S(═O)₂—, and piperidinyl C(═O)—;
R¹³, at each occurrence, is independently selected from
   H, OH, C₁-C₆ alkyl, benzyl, phenethyl,
   (C₁-C₆ alkyl)-C(═O)—, and (C₁-C₆ alkyl)-S(═O)₂—;
alternatively, R¹² and R¹³ together with the nitrogen to which they are attached, may combine to form a 4 to 7 membered ring wherein said 4 to 7 membered ring optionally contains an additional heteroatom selected from O and NH;
R¹⁴, at each occurrence, is independently selected from H, C₁-C₆ alkyl, benzyl, phenethyl, (C₁-C₆ alkyl)-C(═O)—, and (C₁-C₆ alkyl)-S(═O)₂—;
R¹⁵, at each occurrence, is independently selected from
   H, OH, C₁-C₆ alkyl, benzyl, phenethyl, (C₁-C₆ alkyl)-OC(═O)—,
   (C₁-C₆ alkyl)-C(═O)—, and (C₁-C₆ alkyl)-S(═O)₂—; and
alternatively, R¹⁴ and R¹⁵, may combine together with the nitrogen to
   which they are attached, to form a 4 to 7 membered ring,
      wherein said 4 to 7 membered ring optionally contains
      an heteroatom selected from O and NH.
[6] Another embodiment of the invention provides a compound according to Formula (VI),

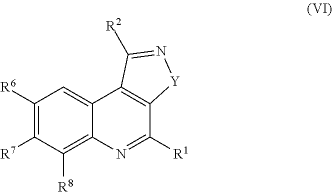

or a stereoisomer, prodrug, or pharmaceutically acceptable salt form, or corresponding polymorph thereof, wherein:
Y is CR₃, O, N^{R3},
R¹ is H, OH,
   C₁-C₈ alkyl substituted with 0-3 R^{1a},
   C₂-C₈ alkenyl substituted with 0-3 R^{1a},
   C₂-C₈ alkynyl substituted with 0-3 R^{1a},
   C₂-C₈ alkoxy substituted with 0-3 R^{1a},
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, or
   5 to 10 membered heterocycle containing 1 to 4 heteroatoms selected from nitrogen, oxygen, and sulfur, wherein said 5 to 10 membered heterocycle is substituted with 0-3 R^{1b},
R^{1a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, and
   5 to 10 membered heterocycle containing 1 to 4 heteroatoms selected from nitrogen, oxygen, and sulphur, wherein said 5 to 10 membered heterocycle is substituted with 0-3 R^{1b};
R^{1b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R² is H,
   C₁-C₈ alkyl substituted with 0-3 R^{2a},
   C₂-C₈ alkenyl substituted with 0-3 R^{2a},
   C₂-C₇ alkynyl substituted with 0-3 R^{2a},
   C₃-C₁₀ carbocycle substituted with 0-3 R^{2b},
   C₁-C₄ sulfonamido substituted with 0-3 R^{2b},
   C₆-C₁₀ aryl substituted with 0-3 R^{2b}, or
   5 to 10 membered heterocycle containing 1 to 4 heteroatoms selected from nitrogen, oxygen, and sulfur, wherein said 5 to 10 membered heterocycle is substituted with 0-3 R^{2b},
R^{2a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{2b},
   C₁-C₄ sulfonamido substituted with 0-3 R^{2b},
   C₆-C₁₀ aryl substituted with 0-3 R^{2b}, and
   5 to 10 membered heterocycle containing 1 to 4 heteroatoms selected from nitrogen, oxygen, and sulphur, wherein said 5 to 10 membered heterocycle is substituted with 0-3 R^{2b};
R^{2b}, at each occurrence, is independently selected from H, OH, Cl, F, Br, I, CN, NO₂, thiazole, NR¹²R¹³, CF₃, acetyl, SCH₃, S(═O)CH₃, S(═O)₂CH₃, H₂N—C(═O)—,
   C₁-C₆ alkyl, C₁-C₄ alkoxy, C₁-C₄ haloalkyl, C₁-C₆ cyanoalkyl,
   C₁-C₄ haloalkoxy, C₁-C₄ cyanoalkoxy, and C₁-C₄ haloalkyl-S—;
R³ is H, O,
   C₁-C₈ alkyl substituted with 0-3 R^{3a},
   C₂-C₈ alkenyl substituted with 0-3 R^{3a},
   C₂-C₈ alkynyl substituted with 0-3 R^{3a}
   C₃-C₁₀ carbocycle substituted with 0-3 R^{3b},
   C₁-C₄ sulfonamido substituted with 0-3 R^{3b},
   aryl substituted with 0-3 R^{3b}, or
   5 to 10 member heterocycle containing 1 to 4 heteroatoms selected from nitrogen, oxygen, and sulfur, wherein said 5 to 10 member heterocycle is substituted with 0-3 R^{3b},
R^{3a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{3b},
   C₁-C₄ sulfonamido substituted with 0-3 R^{3b},
   C₆-C₁₀ aryl substituted with 0-3 R^{3b}, and
   5 to 10 member heterocycle containing 1 to 4 heteroatoms selected from nitrogen, oxygen, and sulphur, wherein said 5 to 10 member heterocycle is substituted with 0-3 R^{3b};
R^{3b}, at each occurrence, is independently selected from H, OH, Cl, F, Br, I, CN, NO₂, NR¹²R¹³, CF₃, acetyl, SCH₃, S(═O)CH₃, S(═O)₂CH₃, H₂N—C(═O)—,
   C₁-C₆ alkyl, C₁-C₄ alkoxy, C₁-C₄ haloalkyl, C₁-C₆ cyanoalkyl,
   C₁-C₄ haloalkoxy, C₁-C₄ cyanoalkoxy, and C₁-C₄ haloalkyl-S—;
R⁴ is H, phenyl, benzyl, C₁-C₄ alkyl, C₃-C₈ cycloalkyl substituted with 0-3 R^{1b}, or
a 5 to 10 membered heterocycle containing 1 to 4 heteroatoms selected from nitrogen, oxygen, and sulphur, wherein said 5 to 10 membered heterocycle is substituted with 0-3 R^{1b};
R⁵ is H, phenyl, benzyl, or C₁-C₄ alkyl;
R⁶ is H,
   C₁-C₈ alkyl substituted with 0-3 R^{6a},
   C₂-C₈ alkenyl substituted with 0-3 R^{6a},
   C₂-C₈ alkynyl substituted with 0-3 R^{6a}
   C₃-C₁₀ carbocycle substituted with 0-3 R^{6b},
   C₁-C₄ sulfonamido substituted with 0-3 R^{6b},
   C₆-C₁₀ aryl substituted with 0-3 R^{6a}; arylamine substituted with 0-3 R^{6a},
   C₁-C₆ alkyloxy substituted with 0-3 R^{6a}, or
   5 to 10 membered heterocycle containing 1 to 4 heteroatoms selected from nitrogen, oxygen, and sulfur, wherein said 5 to 10 membered heterocycle is substituted with 0-3 R^{6b},
R^{6a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R⁶,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, and
   5 to 10 membered heterocycle containing 1 to 4 heteroatoms selected from nitrogen, oxygen, and sulphur, wherein said 5 to 10 membered heterocycle is substituted with 0-3 R^{1b};
R^{6b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R⁷ is H, C₁-C₄ alkyl, C₂-C₄ alkenyl, or C₃-C₄ alkynyl;
R⁸ is H,
   C₁-C₈ alkyl substituted with 0-3 R^{8a},
   C₂-C₈ alkenyl substituted with 0-3 R^{8a},
   C₂-C₈ alkynyl substituted with 0-3 R^{8a}
   C₃-C₁₀ carbocycle substituted with 0-3 R^{8b},
   C₁-C₄ sulfonamido substituted with 0-3 R^{8b},
   C₆-C₁₀ aryl substituted with 0-3 R^{8b}, or
   5 to 10 membered heterocycle containing 1 to 4 heteroatoms selected from nitrogen, oxygen, and sulfur, wherein said 5 to 10 membered heterocycle is substituted with 0-3 R^{8b},
R^{8a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(′O)₂R⁴,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{8b},
   C₁-C₄ sulfonamido substituted with 0-3 R^{8b},
   C₆-C₁₀ aryl substituted with 0-3 R^{8b}, and
   5 to 10 membered heterocycle containing 1 to 4 heteroatoms selected from nitrogen, oxygen, and sulphur, wherein said 5 to 10 membered heterocycle is substituted with 0-3 R^{8b};
R^{8b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R¹², at each occurrence, is independently selected from H, C₁-C₆ alkyl, benzyl, phenethyl, (C₁-C₆ alkyl)-C(═O)—, (C₁-C₆ alkyl)-OC(═O)—, (C₁-C₆ alkyl)-S(═O)₂—, and piperdinyl C(═O)—;
R¹³, at each occurrence, is independently selected from
   H, OH, C₁-C₆ alkyl, benzyl, phenethyl,
   (C₁-C₆ alkyl)-C(═O)—, and (C₁-C₆ alkyl)-S(═O)₂—;
alternatively, R¹² and R¹³ together with the nitrogen to which they are attached, may combine to form a 4 to 7 membered ring wherein said 4 to 7 membered ring optionally contains an additional heteroatom selected from O and NH;
R¹⁴, at each occurrence, is independently selected from H, C₁-C₆ alkyl, benzyl, phenethyl, (C₁-C₆ alkyl)-C(═O)—, and (C₁-C₆ alkyl)-S(═O)₂—;
R¹⁵, at each occurrence, is independently selected from
   H, OH, C₁-C₆ alkyl, benzyl, phenethyl, (C₁-C₆ alkyl)-OC(═O)—,
   (C₁-C₆ alkyl)-C(═O)—, and (C₁-C₆ alkyl)-S(═O)₂—; and
alternatively, R¹⁴ and R¹⁵, may combine together with the nitrogen to
   which they are attached, to form a 4 to 7 membered ring,
      wherein said 4 to 7 membered ring optionally contains
      an heteroatom selected from O and NH.
[7] Another embodiment of the invention provides a compound according to Formula (VII),

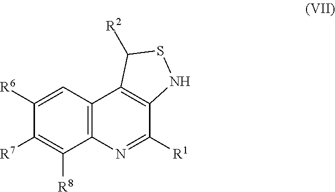

or a stereoisomer, prodrug, pharmaceutically acceptable salt form, or corresponding polymorph thereof, wherein:
R¹ is H, OH,
   C₁-C₈ alkyl substituted with 0-3 R^{1a},
   C₂-C₈ alkenyl substituted with 0-3 R^{1a},
   C₂-C₈ alkynyl substituted with 0-3 R^{1a},
   C₂-C₈ alkoxy substituted with 0-3 R^{1a},
   C₃-C₁₀ carbocycle substituted with 0-3 R^{1b},
   C₁-C₄ sulfonamido substituted with 0-3 R^{1b},
   C₆-C₁₉ aryl substituted with 0-3 R^{1b}, or
   5 to 10 membered heterocycle containing 1 to 4 heteroatoms selected from nitrogen, oxygen, and sulfur, wherein said 5 to 10 membered heterocycle is substituted with 0-3 R^{1b},
R^{1a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, and
   5 to 10 membered heterocycle containing 1 to 4 heteroatoms selected from nitrogen, oxygen, and sulphur, wherein said 5 to 10 membered heterocycle is substituted with 0-3 R^{1b};
R^{1b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R² is H,
   C₁-C₈ alkyl substituted with 0-3 R^{2a},
   C₂-C₈ alkenyl substituted with 0-3 R^{2a},
   C₂-C₈ alkynyl substituted with 0-3 R^{2a},
   C₃-C₁₀ carbocycle substituted with 0-3 R^{2b},
   C₁-C₄ sulfonamido substituted with 0-3 R^{2b},
   C₆-C₁₀ aryl substituted with 0-3 R^{2b}, or
   5 to 10 membered heterocycle containing 1 to 4 heteroatoms selected from nitrogen, oxygen, and sulfur, wherein said 5 to 10 membered heterocycle is substituted with 0-3 R^{2b},
R^{2a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{2b},
   C₁-C₄ sulfonamido substituted with 0-3 R^{2b},
   C₆-C₁₀ aryl substituted with 0-3 R^{2b}, and
   5 to 10 membered heterocycle containing 1 to 4 heteroatoms selected from nitrogen, oxygen, and sulphur, wherein said 5 to 10 membered heterocycle is substituted with 0-3 R^{2b};
R^{2b}, at each occurrence, is independently selected from H, OH, Cl, F, Br, I, CN, NO₂, thiazole, N¹²R¹², CF₃, acetyl, SCH₃, S(═O)CH₃, S(═O)₂CH₃, H₂N—C(═O)—,
   C₁-C₆ alkyl, C₁-C₄ alkoxy, C₁-C₄ haloalkyl, C₁-C₆ cyanoalkyl,
   C₁-C₄ haloalkoxy, C₁-C₄ cyanoalkoxy, and C₁-C₄ haloalkyl-S—;
R⁴ is H, phenyl, benzyl, C₁-C₄ alkyl, C₃-C₈ cycloalkyl substituted with 0-3 R^{1b}, or
a 5 to 10 membered heterocycle containing 1 to 4 heteroatoms selected from nitrogen, oxygen, and sulphur, wherein said 5 to 10 membered heterocycle is substituted with 0-3 R^{1b};
R⁵ is H, phenyl, benzyl, or C₁-C₄ alkyl;
R⁶ is H,
   C₁-C₈ alkyl substituted with 0-3 R^{6a},
   C₂-C₈ alkenyl substituted with 0-3 R^{6a},
   C₂-C₈ alkynyl substituted with 0-3 R^{6a}
   C₃-C₁₀ carbocycle substituted with 0-3 R^{6b},
   C₁-C₄ sulfonamido substituted with 0-3 R^{6b},
   C₆-C₁₀ aryl substituted with 0-3 R^{6a}; arylamine substituted with 0-3 R^{6a},
   C₁-C₆ alkyloxy substituted with 0-3 R^{6a}, or
   5 to 10 membered heterocycle containing 1 to 4 heteroatoms selected from nitrogen, oxygen, and sulfur, wherein said 5 to 10 membered heterocycle is substituted with 0-3 R^{6b},
R^{6a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R⁶,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, and
   5 to 10 membered heterocycle containing 1 to 4 heteroatoms selected from nitrogen, oxygen, and sulphur, wherein said 5 to 10 membered heterocycle is substituted with 0-3 R^{1b};
R^{6b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R⁷ is H, C₁-C₄ alkyl, C₂-C₄ alkenyl, or C₃-C₄ alkynyl;
R⁸ is H,
   C₁-C₈ alkyl substituted with 0-3 R^{8a},
   C₂-C₈ alkenyl substituted with 0-3 R^{8a},
   C₂-C₈ alkynyl substituted with 0-3 R^{8a}
   C₃-C₁₀ carbocycle substituted with 0-3 R^{8b},
   C₁-C₄ sulfonamido substituted with 0-3 R^{8b},
   C₆-C₁₀ aryl substituted with 0-3 R^{8b}, or
   5 to 10 membered heterocycle containing 1 to 4 heteroatoms selected from nitrogen, oxygen, and sulfur, wherein said 5 to 10 membered heterocycle is substituted with 0-3 R^{8b},
R^{8a}, at each occurrence, is independently selected from is H, Cl, F, Br, I, CN, NO₂, NR¹²R¹³, OR⁵, SR⁴, C(═O)R₄, NR¹⁴R¹⁵, S(═O)R⁶, S(═O)₂R⁴,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{8b},
   C₁-C₄ sulfonamido substituted with 0-3 R^{8b},
   C₆-C₁₀ aryl substituted with 0-3 R^{8b}, and
   5 to 10 membered heterocycle containing 1 to 4 heteroatoms selected from nitrogen, oxygen, and sulphur, wherein said 5 to 10 membered heterocycle is substituted with 0-3 R^{8b};
R^{8b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R¹², at each occurrence, is independently selected from H, C₁-C₆ alkyl, benzyl, phenethyl, (C₁-C₆ alkyl)-C(═O)—, (C₁-C₆ alkyl)-OC(═O)—, (C₁-C₆ alkyl)-S(═O)₂—, and piperdinyl C(═O)—;
R¹³, at each occurrence, is independently selected from
   H, OH, C₁-C₆ alkyl, benzyl, phenethyl,
   (C₁-C₆ alkyl)-C(═O)—, and (C₁-C₆ alkyl)-S(═O)₂—;
alternatively, R¹² and R¹³ together with the nitrogen to which they are attached, may combine to form a 4 to 7 membered ring wherein said 4 to 7 membered ring optionally contains an additional heteroatom selected from O and NH;
R¹⁴, at each occurrence, is independently selected from H, C₁-C₆ alkyl, benzyl, phenethyl, (C₁-C₆ alkyl)-C(═O)—, and (C₁-C₆ alkyl)-S(═O)₂—;
R¹⁵, at each occurrence, is independently selected from
   H, OH, C₁-C₆ alkyl, benzyl, phenethyl, (C₁-C₆ alkyl)-OC(═O)—,
   (C₁-C₆ alkyl)-C(═O)—, and (C₁-C₆ alkyl)-S(═O)₂—; and
alternatively, R¹⁴ and R¹⁵, may combine together with the nitrogen to
   which they are attached, to form a 4 to 7 membered ring,
      wherein said 4 to 7 membered ring optionally contains
      an heteroatom selected from O and NH.
Compounds according to Formula (VII) may have an alternate structure according to Formula (VIIa) shown below:

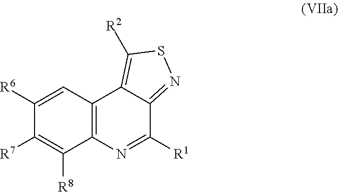

[8] An embodiment of the invention provides a compound according to Formula (VIII),

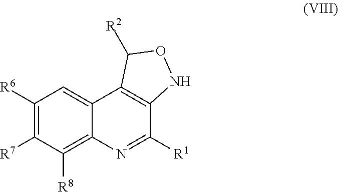

or a stereoisomer, prodrug, pharmaceutically acceptable salt form, or corresponding polymorph thereof, wherein:
R¹ is H, OH,
   C₁-C₈ alkyl substituted with 0-3 R^{1a},
   C₂-C₈ alkenyl substituted with 0-3 R^{1a},
   C₂-C₈ alkynyl substituted with 0-3 R^{1a},
   C₂-C₈ alkoxy substituted with 0-3 R^{1a},
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, or
   5 to 10 membered heterocycle containing 1 to 4 heteroatoms selected from nitrogen, oxygen, and sulfur, wherein said 5 to 10 membered heterocycle is substituted with 0-3 R^{1b},
R^{1a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, and
   5 to 10 membered heterocycle containing 1 to 4 heteroatoms selected from nitrogen, oxygen, and sulphur, wherein said 5 to 10 membered heterocycle is substituted with 0-3 R^{1b};
R^{1b}, at each occurrence, is independently selected from H, OH, Cl, F, Br, I, CN, NO2, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R² is H,
   C₁-C₈ alkyl substituted with 0-3 R^{2a},
   C₂-C₈ alkenyl substituted with 0-3 R^{2a},
   C₂-C₈ alkynyl substituted with 0-3 R^{2a},
   C₃-C₁₀ carbocycle substituted with 0-3 R^{2b},
   C₁-C₄ sulfonamido substituted with 0-3 R^{2b},
   C₆-C₁₀ aryl substituted with 0-3 R^{2b}, or
   5 to 10 membered heterocycle containing 1 to 4 heteroatoms selected from nitrogen, oxygen, and sulfur, wherein said 5 to 10 membered heterocycle is substituted with 0-3 R^{2b},
R^{2a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{2b},
   C₁-C₄ sulfonamido substituted with 0-3 R^{2b},
   C₆-C₁₀ aryl substituted with 0-3 R^{2b}, and
   5 to 10 membered heterocycle containing 1 to 4 heteroatoms selected from nitrogen, oxygen, and sulphur, wherein said 5 to 10 membered heterocycle is substituted with 0-3 R^{2b};
R^{2b}, at each occurrence, is independently selected from H, OH, Cl, F, Br, I, CN, NO₂, thiazole, NR₁₂R₁₃, CF₃, acetyl, SCH₃, S(═O)CH₃, S(═O)₂CH₃, H₂N—C(═O)—,
   C₁-C₆ alkyl, C₁-C₄ alkoxy, C₁-C₄ haloalkyl, C₁-C₆ cyanoalkyl,
   C₁-C₄ haloalkoxy, C₁-C₄ cyanoalkoxy, and C₁-C₄ haloalkyl-S—;
R⁴ is H, phenyl, benzyl, C₁-C₄ alkyl, C₃-C₈ cycloalkyl substituted with 0-3 R^{1b}, or
a 5 to 10 membered heterocycle containing 1 to 4 heteroatoms selected from nitrogen, oxygen, and sulphur, wherein said 5 to 10 membered heterocycle is substituted with 0-3 R^{1b};
R⁵ is H, phenyl, benzyl, or C₁-C₄ alkyl;
R⁶ is H,
   C₁-C₈ alkyl substituted with 0-3 R^{6a},
   C₂-C₈ alkenyl substituted with 0-3 R^{6a},
   C₂-C₈ alkynyl substituted with 0-3 R^{6a}
   C₃-C₁₀ carbocycle substituted with 0-3 R^{6b},
   C₁-C₄ sulfonamido substituted with 0-3 R^{6b},
   C₆-C₁₀ aryl substituted with 0-3 R^{6a}; arylamine substituted with 0-3 R^{6a},
   C₁-C₆ alkyloxy substituted with 0-3 R^{6a}, or
   5 to 10 membered heterocycle containing 1 to 4 heteroatoms selected from nitrogen, oxygen, and sulfur, wherein said 5 to 10 membered heterocycle is substituted with 0-3 R^{6b},
R^{6a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R⁶,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{1b},
   C₁-C₄ sulfonamido substituted with 0-3 R^{1b},
   C₆-C₁₀ aryl substituted with 0-3 R^{1b}, and
   5 to 10 membered heterocycle containing 1 to 4 heteroatoms selected from nitrogen, oxygen, and sulphur, wherein said 5 to 10 membered heterocycle is substituted with 0-3 R^{1b};
R^{6b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R⁷ is H, C₁-C₄ alkyl, C₂-C₄ alkenyl, or C₃-C₄ alkynyl;
R⁸ is H,
   C₁-C₈ alkyl substituted with 0-3 R^{8a},
   C₂-C₈ alkenyl substituted with 0-3 R^{8a},
   C₂-C₈ alkynyl substituted with 0-3 R^{8a}
   C₃-C₁₀ carbocycle substituted with 0-3 R^{8b},
   C₁-C₄ sulfonamido substituted with 0-3 R^{8b},
   C₆-C₁₀ aryl substituted with 0-3 R^{8b}, or
   5 to 10 membered heterocycle containing 1 to 4 heteroatoms selected from nitrogen, oxygen, and sulfur, wherein said 5 to 10 membered heterocycle is substituted with 0-3 R^{8b},
R^{8a}, at each occurrence, is independently selected from is H, Cl, F, Br, I, CN, NO₂, NR¹²R¹³, OR⁵, SR⁴, C(═O)R₄, NR¹⁴R¹⁵, S(═O)R⁶, S(═O)₂R⁴,
   C₁-C₆ alkyl, C₁-C₄ alkoxy, C₁-C₄ haloalkyl,
   C₁-C₄ haloalkoxy, C₁-C₄ haloalkyl-S—,
   C₃-C₁₀ carbocycle substituted with 0-3 R^{8b},
   C₁-C₄ sulfonamido substituted with 0-3 R^{8b},
   C₆-C₁₀ aryl substituted with 0-3 R^{8b}, and
   5 to 10 membered heterocycle containing 1 to 4 heteroatoms selected from nitrogen, oxygen, and sulphur, wherein said 5 to 10 membered heterocycle is substituted with 0-3 R^{8b};
R^{8b}, at each occurrence, is independently selected from H, OH, Cl, F, Br, I, CN, NO₂, NR¹²R¹³, CF₃, acetyl, SCH₃, S(═O)CH₃, S(═O)₂CH₃,
   C₁-C₆ alkyl, C₁-C₄ alkoxy, C₁-C₄ haloalkyl,
   C₁-C₄ haloalkoxy, and C₁-C₄ haloalkyl-S—;
R¹², at each occurrence, is independently selected from H, C₁-C₆ alkyl, benzyl, phenethyl, (C₁-C₆ alkyl)-C(═O)—, (C₁-C₆ alkyl)-OC(═O)—, (C₁-C₆ alkyl)-S(═O)₂—, and piperdinyl C(═O)—;
R¹³, at each occurrence, is independently selected from
   H, OH, C₁-C₆ alkyl, benzyl, phenethyl,
   (C₁-C₆ alkyl)-C(═O)—, and (C₁-C₆ alkyl)-S(═O)₂—;
alternatively, R¹² and R¹³ together with the nitrogen to which they are attached, may combine to form a 4 to 7 membered ring wherein said 4 to 7 membered ring optionally contains an additional heteroatom selected from O and NH;
R¹⁴, at each occurrence, is independently selected from H, C₁-C₆ alkyl, benzyl, phenethyl, (C₁-C₆ alkyl)-C(═O)—, and (C₁-C₆ alkyl)-S(═O)₂—;
R¹⁵, at each occurrence, is independently selected from
   H, OH, C₁-C₆ alkyl, benzyl, phenethyl, (C₁-C₆ alkyl)-OC(═O)—,
   (C₁-C₆ alkyl)-C(═O)—, and (C ₗ-C₆ alkyl)-S(═O)₂—; and
alternatively, R¹⁴ and R¹⁵, may combine together with the nitrogen to
   which they are attached, to form a 4 to 7 membered ring,
      wherein said 4 to 7 membered ring optionally contains
      an heteroatom selected from O and NH.
Compounds according to Formula (VIII) may have an alternate structure according to Formula (VIIa) shown below:

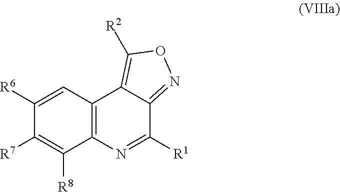

[9] An embodiment of the invention provides a compound according to Formula (II), or a stereoisomer or a pharmaceutically acceptable salt form or prodrug or corresponding polymorphs thereof, is shown below:

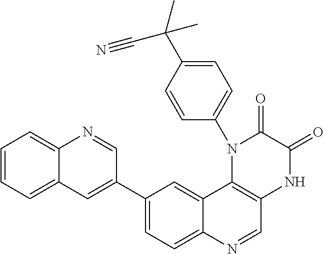

2-(4-(2,3-dioxo-9-(quinolin-3-yl)-3,4-dihydropyrazino[2,3-c]quinolin-1(2H)-yl)phenyl)-2-methylpropanenitrile (1026);

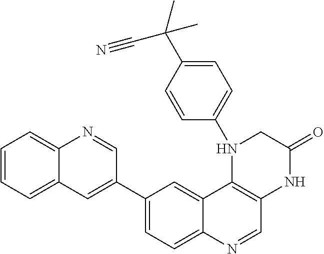

2-methyl-2-(4-(3-oxo-9-(quinolin-3-yl)-3,4-dihydropyrazino[2,3-c]quinolin-1(2H)-yl)phenyl)propanenitrile (1029)
[10] An embodiment of the invention provides a compound (A) according to Formula (V) is shown below:

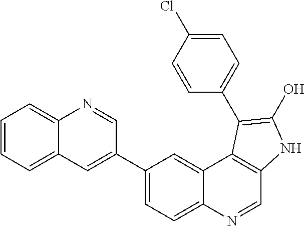

1-(4-chlorophenyl)-8-(quinolin-3-yl)-3H-pyrrolo[2,3-c]quinolin-2-ol (1050)
or a stereoisomer or pharmaceutically acceptable salt forms or prodrug or polymorphs thereof.
[11] Particular embodiments of the invention provides a compound according to Formula (VI), or a stereoisomer or a pharmaceutically acceptable salt form or prodrug or polymorph thereof, selected from:

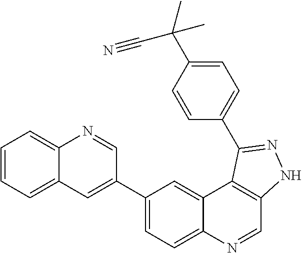

2-methyl-2-(4-(8-(quinolin-3-yl)-3H-pyrazolo[3,4-c]quinolin-1-yl)phenyl)propanenitrile (1078),
or a stereoisomer, pharmaceutically acceptable salt forms or prodrug or polymorph thereof;

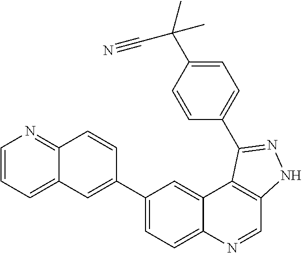

2-methyl-2-(4-(8-(quinolin-6-yl)-3H-pyrazolo[3,4-c]quinolin-1-yl)phenyl)propanenitrile (1110),
or a stereoisomer, pharmaceutically acceptable salt form or prodrug or polymorph thereof;

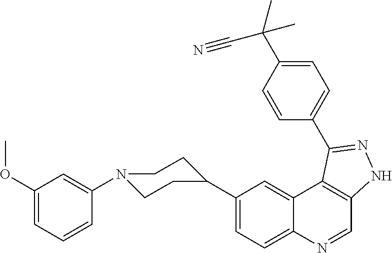

2-(4-(8-(1-(3-methoxyphenyl)piperidin-4-yl)-3H-pyrazolo[3,4-c]quinolin-1-yl)phenyl)-2-methylpropanenitrile (1080), or a stereoisomer, pharmaceutically acceptable salt form or prodrug or polymorph thereof;

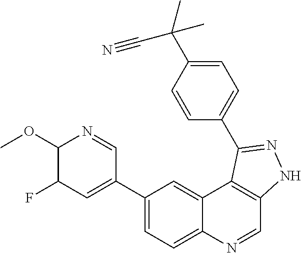

2-(4-(8-(5-fluoro-6-methoxy-5,6-dihydropyridin-3-yl)-3H-pyrazolo[3,4-c]quinolin-1-yl)phenyl)-2-methylpropanenitrile (1081), or a stereoisomer or pharmaceutically acceptable salt form or prodrug or polymorph thereof;

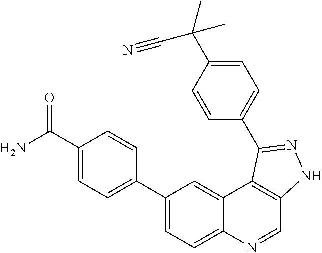

4-(1-(4-(2-cyanopropan-2-yl)phenyl)-3H-pyrazolo[3,4-c]quinolin-8-yl)benzamide (1083),
or a stereoisomer or pharmaceutically acceptable salt form or prodrug or polymorph thereof;

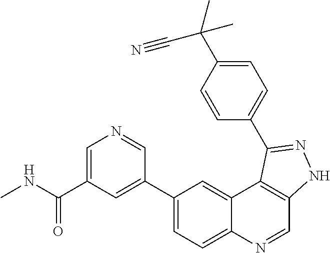

5-(1-(4-(2-cyanopropan-2-yl)phenyl)-3H-pyrazolo[3,4-c]quinolin-8-yl)-N-methylnicotinamide (1085), or a stereoisomer or pharmaceutically acceptable salt form or prodrug or polymorph thereof;

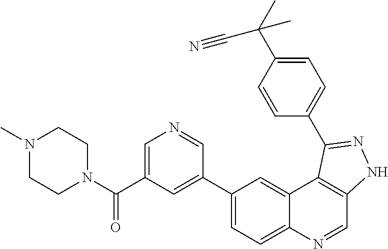

2-methyl-2-(4-(8-(5-(4-methylpiperazine-1-carbonyl)pyridin-3-yl)-3H-pyrazolo[3,4-c]quinolin-1-yl)phenyl)propanenitrile (1087), or a stereoisomer or pharmaceutically acceptable salt forms or prodrug or polymorph thereof;

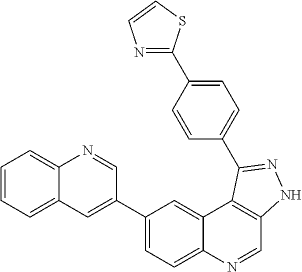

2-(4-(8-(quinolin-3-yl)-3H-pyrazolo[3,4-c]quinolin-1-yl)phenyl)thiazole (1089),
or a stereoisomer or pharmaceutically acceptable salt form or prodrug or polymorph thereof;

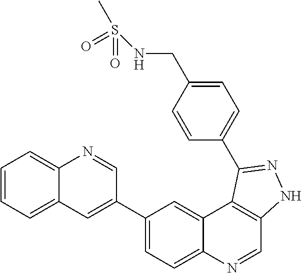

N-(4-(8-(quinolin-3-yl)-3H-pyrazolo[3,4-c]quinolin-1-yl)benzyl)methanesulfonamide (1091),
or a stereoisomer or pharmaceutically acceptable salt form or prodrug or polymorph thereof;

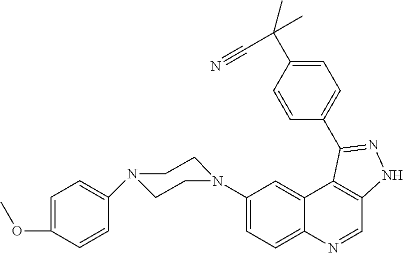

2-(4-(8-(4-(4-methoxyphenyl)piperazin-1-yl)-3H-pyrazolo[3,4-c]quinolin-1-yl)phenyl)-2-methylpropanenitrile (1092), or a stereoisomer or pharmaceutically acceptable salt form or prodrug or polymorph thereof;

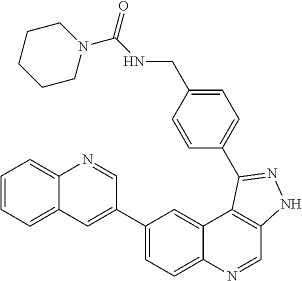

N-(4-(8-(quinolin-3-yl)-3H-pyrazolo[3,4-c]quinolin-1-yl)benzyl)piperidine-1-carboxamide (1093), or a stereoisomer or pharmaceutically acceptable salt forms or prodrug or polymorph thereof;

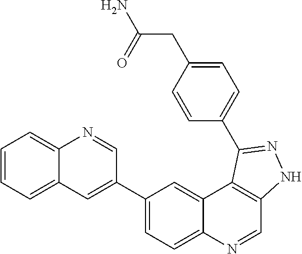

2-(4-(8-(quinolin-3-yl)-3H-pyrazolo[3,4-c]quinolin-1-yl)phenyl)acetamide (1094),
or a stereoisomer or pharmaceutically acceptable salt form or prodrug or polymorph thereof;

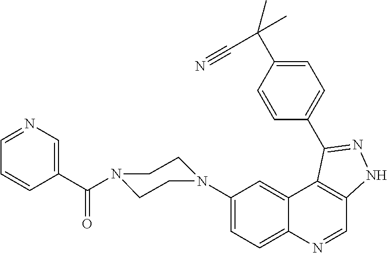

2-methyl-2-(4-(8-(4-nicotinoylpiperazin-1-yl)-3H-pyrazolo[3,4-c]quinolin-1-yl)phenyl)propanenitrile (1095),
or a stereoisomer or pharmaceutically acceptable salt form or prodrug or polymorph thereof;

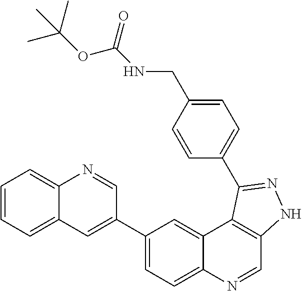

tert-butyl 4-(8-(quinolin-3-yl)-3H-pyrazolo[3,4-c]quinolin-1-yl)benzylcarbamate (1096),
or a stereoisomer or pharmaceutically acceptable salt form or prodrug or polymorph thereof;

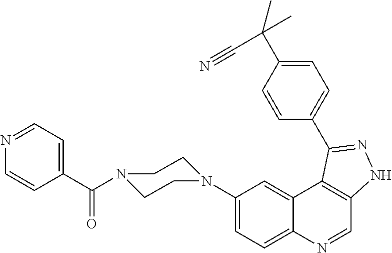

2-(4-(8-(4-isonicotinoylpiperazin-1-yl)-3H-pyrazolo[3,4-c]quinolin-1-yl)phenyl)-2-methylpropanenitrile (1097), or a stereoisomer or pharmaceutically acceptable salt form or prodrug or polymorph thereof;

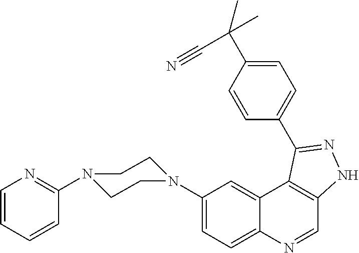

2-methyl-2-(4-(8-(4-(pyridin-2-yl)piperazin-1-yl)-3H-pyrazolo[3,4-c]quinolin-1-yl)phenyl)propanenitrile (1098), or a stereoisomer or pharmaceutically acceptable salt form or prodrug or polymorph thereof;

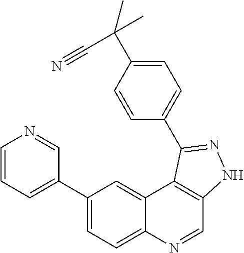

2-methyl-2-(4-(8-(pyridin-3-yl)-3H-pyrazolo[3,4-c]quinolin-1-yl)phenyl)propanenitrile (1107), or a stereoisomer or pharmaceutically acceptable salt form or prodrug or polymorph thereof; and

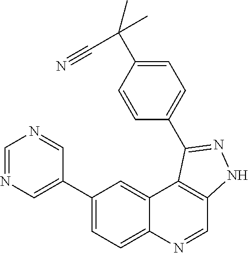

2-methyl-2-(4-(8-(pyrimidin-5-yl)-3H-pyrazolo[3,4-c]quinolin-1-yl)phenyl)propanenitrile (1108), or a stereoisomer or pharmaceutically acceptable salt form or prodrug or polymorph thereof;

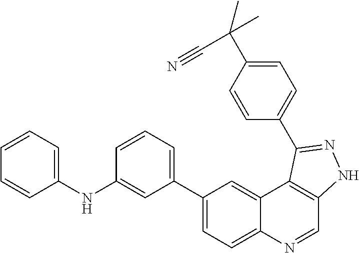

2-methyl-2-(4-(8-(3-(phenylamino)phenyl)-3H-pyrazolo[3,4-c]quinolin-1-yl)phenyl)propanenitrile (1119), or a stereoisomer or pharmaceutically acceptable salt form or prodrug or polymorph thereof;

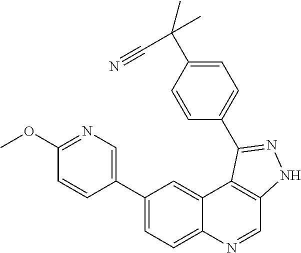

2-(4-(8-(6-methoxypyridin-3-yl)-3H-pyrazolo[3,4-c]quinolin-1-yl)phenyl)-2-methylpropanenitrile (1118), or a stereoisomer or pharmaceutically acceptable salt form or prodrug or polymorph thereof;

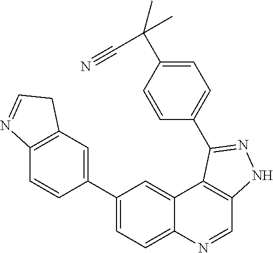

2-(4-(8-(3H-indol-5-yl)-3H-pyrazolo[3,4-c]quinolin-1-yl)phenyl)-2-methylpropanenitrile (1114), or a stereoisomer or pharmaceutically acceptable salt form or prodrug or polymorph thereof;

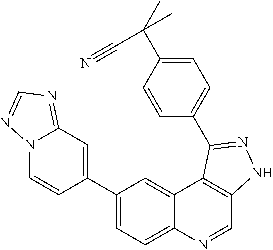

2-(4-(8-(1,3a-dihydro-[1,2,3]triazolo[1,5-a]pyridin-5-yl)-3H-pyrazolo[3,4-c]quinolin-1-yl)phenyl)-2-methylpropanenitrile (1111), or a stereoisomer or pharmaceutically acceptable salt form or prodrug or polymorph thereof;

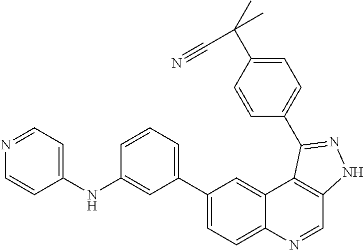

2-methyl-2-(4-(8-(3-(pyridin-4-ylamino)phenyl)-3H-pyrazolo[3,4-c]quinolin-1-yl)phenyl)propanenitrile (1121), or a stereoisomer or pharmaceutically acceptable salt form or prodrug or polymorph thereof;

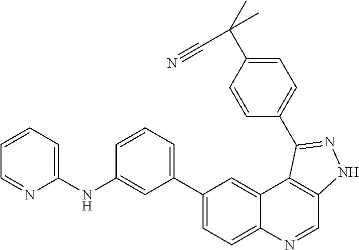

2-methyl-2-(4-(8-(3-(pyridin-2-ylamino)phenyl)-3H-pyrazolo[3,4-c]quinolin-1-yl)phenyl)propanenitrile (1120), or a stereoisomer or pharmaceutically acceptable salt form or prodrug or polymorph thereof;

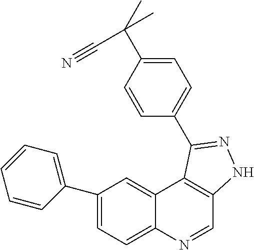

2-methyl-2-(4-(8-phenyl-3H-pyrazolo[3,4-c]quinolin-1-yl)phenyl)propanenitrile (1125),
or a stereoisomer or pharmaceutically acceptable salt form or prodrug or polymorph thereof;

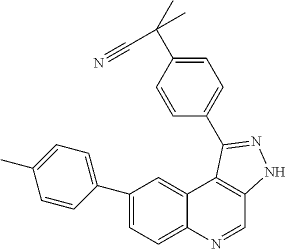

2-methyl-2-(4-(8-p-tolyl-3H-pyrazolo[3,4-c]quinolin-1-yl)phenyl)propanenitrile (1126),
or a stereoisomer or pharmaceutically acceptable salt forms or prodrug or polymorph thereof;

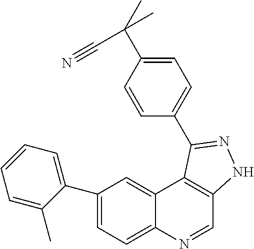

2-methyl-2-(4-(8-o-tolyl-3H-pyrazolo[3,4-c]quinolin-1-yl)phenyl)propanenitrile (1124),
or a stereoisomer or pharmaceutically acceptable salt forms or prodrug or polymorph thereof;

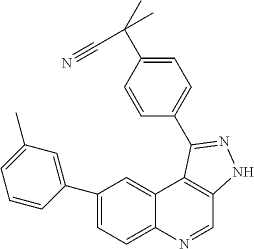

2-methyl-2-(4-(8-m-tolyl-3H-pyrazolo[3,4-c]quinolin-1-yl)phenyl)propanenitrile (1123), or a stereoisomer or pharmaceutically acceptable salt forms or prodrug or polymorph thereof;

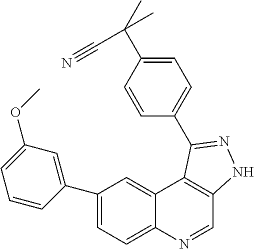

2-(4-(8-(3-methoxyphenyl)-3H-pyrazolo[3,4-c]quinolin-1-yl)phenyl)-2-methylpropanenitrile (1115), or a stereoisomer or pharmaceutically acceptable salt forms or prodrug or polymorph thereof;

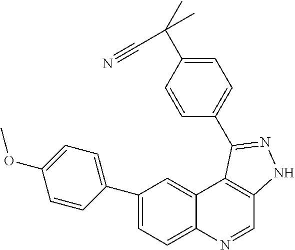

2-(4-(8-(4-methoxyphenyl)-3H-pyrazolo[3,4-c]quinolin-1-yl)phenyl)-2-methylpropanenitrile (1117), or a stereoisomer or pharmaceutically acceptable salt forms or prodrug or polymorph thereof;

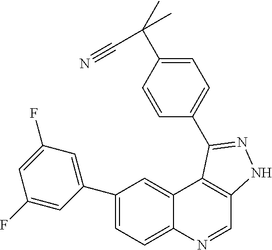

2-(4-(8-(3,5-difluorophenyl)-3H-pyrazolo[3,4-c]quinolin-1-yl)phenyl)-2-methylpropanenitrile (1112), or a stereoisomer or pharmaceutically acceptable salt forms or prodrug or polymorph thereof;

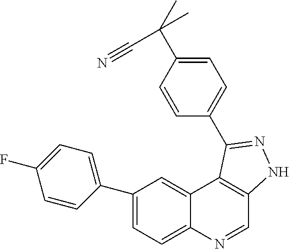

2-(4-(8-(4-fluorophenyl)-3H-pyrazolo[3,4-c]quinolin-1-yl)phenyl)-2-methylpropanenitrile (1116), or a stereoisomer or pharmaceutically acceptable salt forms or prodrug or polymorph thereof;

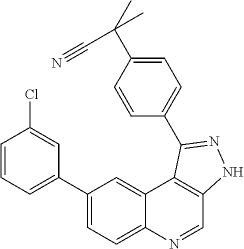

2-(4-(8-(3-chlorophenyl)-3H-pyrazolo[3,4-c]quinolin-1-yl)phenyl)-2-methylpropanenitrile (1113), or a stereoisomer or pharmaceutically acceptable salt forms or prodrug or polymorph thereof.

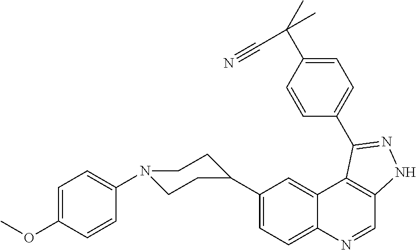

2-(4-(8-(1-(4-methoxyphenyl)piperidin-4-yl)-3H-pyrazolo[3,4-c]quinolin-1-yl)phenyl)-2-methylpropanenitrile (1082)

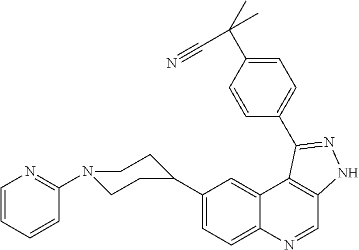

2-methyl-2-(4-(8-(1-(pyridin-2-yl)piperidin-4-yl)-3H-pyrazolo[3,4-c]quinolin-1-yl)phenyl)propanenitrile (1084),

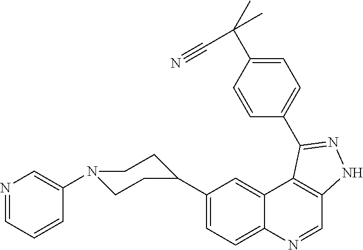

2-methyl-2-(4-(8-(1-(pyridin-3-yl)piperidin-4-yl)-3H-pyrazolo[3,4-c]quinolin-1-yl)phenyl)propanenitrile (1086),

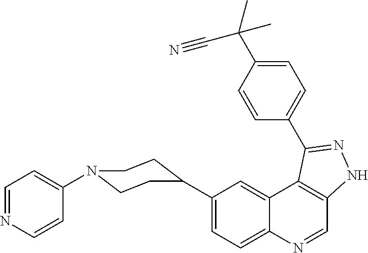

2-methyl-2-(4-(8-(1-(pyridin-4-yl)piperidin-4-yl)-3H-pyrazolo[3,4-c]quinolin-1-yl)phenyl)propanenitrile (1088),

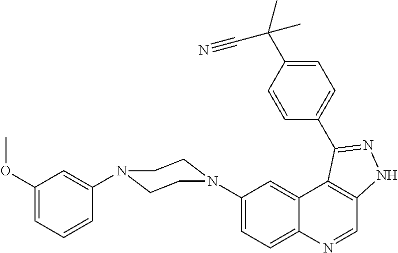

2-(4-(8-(4-(3-methoxyphenyl)piperazin-1-yl)-3H-pyrazolo[3,4-c]quinolin-1-yl)phenyl)-2-methylpropanenitrile (1090),

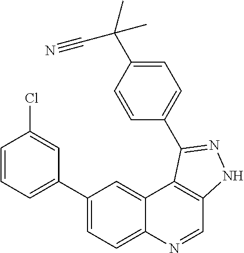

2-(4-(8-(3-chlorophenyl)-3H-pyrazolo[3,4-c]quinolin-1-yl)phenyl)-2-methylpropanenitrile (1113).
An embodiment of the invention provides a compound according to Formula (VII), or a stereoisomer, prodrug, or polymorph, or pharmaceutically acceptable salt form thereof, comprising:

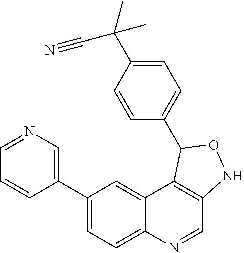

2-methyl-2-(4-(8-(pyridin-3-yl)-1,3-dihydroisoxazolo[3,4-c]quinolin-1-yl)phenyl)propanenitrile (1129).
[12] An embodiment of the invention provides a compound according to Formula (VII), or a stereoisomer, prodrug, or polymorph, or pharmaceutically acceptable salt form thereof, comprising:

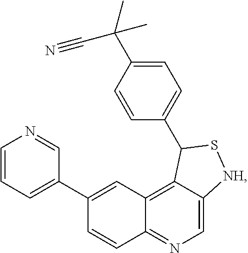

2-methyl-2-(4-(8-(pyridine-3-yl)isothiazolo[3,4-c]quinolin-1-yl)propanenitrile [1122].

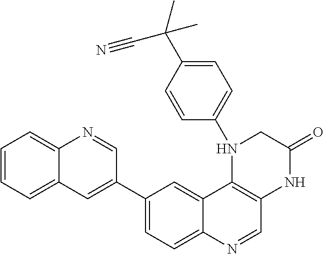

2-methyl-2-(4-(3-oxo-9-(quinolin-3-yl)-3,4-dihydropyrazino[2,3-c]quinolin-1(2H)-yl)phenyl)propanenitrile (1029).

### Summary of Compounds of the Invention is Found in Table A, as Follows.

TABLE A

| S/N | Compound Name |
|---|---|
| 1026 | 2-(4-(2,3-dioxo-9-(quinolin-3-yl)-3,4-dihydropyrazino[2,3-c]quinolin-1(2H)-yl)phenyl)-2- |
| | methylpropanenitrile |
| 1050 | 1-(4-chlorophenyl)-8-(quinolin-3-yl)-3H-pyrrolo[2,3-c]quinolin-2-ol |
| 1078 | 2-methyl-2-(4-(8-(quinolin-3-yl)-3H-pyrazolo[3,4-c]quinolin-1-yl)phenyl)propanenitrile |
| 1080 | 2-(4-(8-(1-(3-methoxyphenyl)piperidin-4-yl)-3H-pyrazolo[3,4-c]quinolin-1-yl)phenyl)-2- |
| | methylpropanenitrile |
| 1081 | 2-(4-(8-(5-fluoro-6-methoxy-5,6-dihydropyridin-3-yl)-3H-pyrazolo[3,4-c]quinolin-1-yl)phenyl)-2- |
| | methylpropanenitrile |
| 1083 | 4-(1-(4-(2-cyanopropan-2-yl)phenyl)-3H-pyrazolo[3,4-c]quinolin-8-yl)benzamide |
| 1085 | 5-(1-(4-(2-cyanopropan-2-yl)phenyl)-3H-pyrazolo[3,4-c]quinolin-8-yl)-N-methylnicotinamide |
| 1087 | 2-methyl-2-(4-(8-(5-(4-methylpiperazine-1-carbonyl)pyridin-3-yl)-3H-pyrazolo[3,4-c]quinolin-1- |
| | yl)phenyl)propanenitrile |
| 1089 | 2-(4-(8-(quinolin-3-yl)-3H-pyrazolo[3,4-c]quinolin-1-yl)phenyl)thiazole |
| 1091 | N-(4-(8-(quinolin-3-yl)-3H-pyrazolo[3,4-c]quinolin-1-yl)benzyl)methanesulfonamide |
| 1092 | 2-(4-(8-(4-(4-methoxyphenyl)piperazin-1-yl)-3H-pyrazolo[3,4-c]quinolin-1-yl)phenyl)-2- |
| | methylpropanenitrile |
| 1093 | N-(4-(8-(quinolin-3-yl)-3H-pyrazolo[3,4-c]quinolin-1-yl)benzyl)piperidine-1-carboxamide |
| 1094 | 2-(4-(8-(quinolin-3-yl)-3H-pyrazolo[3,4-c]quinolin-1-yl)phenyl)acetamide |
| 1095 | 2-methyl-2-(4-(8-(4-nicotinoylpiperazin-1-yl)-3H-pyrazolo[3,4-c]quinolin-1-yl)phenyl)propanenitrile |
| 1096 | tert-butyl 4-(8-(quinolin-3-yl)-3H-pyrazolo[3,4-c]quinolin-1-yl)benzylcarbamate |
| 1097 | 2-(4-(8-(4-isonicotinoylpiperazin-1-yl)-3H-pyrazolo[3,4-c]quinolin-1-yl)phenyl)-2-methylpropanenitrile |
| 1098 | 2-methyl-2-(4-(8-(4-(pyridin-2-yl)piperazin-1-yl)-3H-pyrazolo[3,4-c]quinolin-1-yl)phenyl)propanenitrile |
| 1107 | 2-methyl-2-(4-(8-(pyridin-3-yl)-3H-pyrazolo[3,4-c]quinolin-1-yl)phenyl)propanenitrile |
| 1108 | 2-methyl-2-(4-(8-(pyrimidin-5-yl)-3H-pyrazolo[3,4-c]quinolin-1-yl)phenyl)propanenitrile |
| 1110 | 2-methyl-2-(4-(8-(quinolin-6-yl)-3H-pyrazolo[3,4-c]quinolin-1-yl)phenyl)propanenitrile |
| 1111 | 2-(4-(8-(1,3a-dihydro-[1,2,3]triazolo[1,5-a]pyridin-5-yl)-3H-pyrazolo[3,4-c]quinolin-1-yl)phenyl)-2- |
| | methylpropanenitrile |
| 1112 | 2-(4-(8-(3,5-difluorophenyl)-3H-pyrazolo[3,4-c]quinolin-1-yl)phenyl)-2-methylpropanenitrile |
| 1114 | 2-(4-(8-(3H-indol-5-yl)-3H-pyrazolo[3,4-c]quinolin-1-yl)phenyl)-2-methylpropanenitrile |
| 1115 | 2-(4-(8-(3-methoxyphenyl)-3H-pyrazolo[3,4-c]quinolin-1-yl)phenyl)2-methylpropanenitrile |
| 1116 | 2-(4-(8-(4-fluorophenyl)-3H-pyrazolo[3,4-c]quinolin-1-yl)phenyl)-2-methylpropanenitrile |
| 1117 | 2-(4-(8-(4-methoxyphenyl)-3H-pyrazolo[3,4-c]quinolin-1-yl)phenyl)-2-methylpropanenitrile |
| 1118 | 2-(4-(8-(6-methoxypyridin-3-yl)-3H-pyrazolo[3,4-c]quinolin-1-yl)phenyl)-2-methylpropanenitrile |
| 1119 | 2-methyl-2-(4-(8-(3-(phenylamino)phenyl)-3H-pyrazolo[3,4-c]quinolin-1-yl)phenyl)propanenitrile |
| 1120 | 2-methyl-2-(4-(8-(3-(pyridin-2-ylamino)phenyl)-3H-pyrazolo[3,4-c]quinolin-1-yl)phenyl)propanenitrile |
| 1121 | 2-methyl-2-(4-(8-(3-(pyridin-4-ylamino)phenyl)-3H-pyrazolo[3,4-c]quinolin-1-yl)phenyl)propanenitrile |
| 1122 | 2-methyl-2-(4-(8-(pyridin-3-yl)-1,3-dihydroisothiazolo[3,4-c]quinolin-1-yl)phenyl)propanenitrile |
| 1123 | 2-methyl-2-(4-(8-m-tolyl-3H-pyrazolo[3,4-c]quinolin-1-yl)phenyl)propanenitrile |
| 1124 | 2-methyl-2-(4-(8-o-tolyl-3H-pyrazolo[3,4-c]quinolin-1-yl)phenyl)propanenitrile |
| 1125 | 2-methyl-2-(4-(8-phenyl-3H-pyrazolo[3,4-c]quinolin-1-yl)phenyl)propanenitrile |
| 1126 | 2-methyl-2-(4-(8-p-tolyl-3H-pyrazolo[3,4-c]quinolin-1-yl)phenyl)propanenitrile |
| 1113 | 2-(4-(8-(3-chlorophenyl)-3H-pyrazolo[3,4-c]quinolin-1-yl)phenyl)-2-methylpropanenitrile |
| 1162 | 2-(4-(8-(4-chlorophenyl)-3H-pyrazolo[3,4-c]quinolin-1-yl)phenyl)-2-methylpropanenitrile |
| 1163 | 2-(4-(8-(2-fluorophenyl)-3H-pyrazolo[3,4-c]quinolin-1-yl)phenyl)-2-methylpropanenitrile |
| 1164 | 2-(4-(8-(3-fluorophenyl)-3H-pyrazolo[3,4-c]quinolin-1-yl)phenyl)-2-methytpropanenitrile |
| 1165 | 2-(4-(8-(2-methoxyphenyl)-3H-pyrazolo[3,4-c]quinolin-1-yl)phenyl)-2-methylpropanenitrile |
| 1166 | 2-(4-(8-(1H-pyrazol-4-yl)-3H-pyrazolo[3,4-c]quinolin-1-yl)phenyl)-2-methylpropanenitriIe |
| 1167 | 2-methyl-2-(4-(8-(1-methyl-1H-pyrazol-4-yl)-3H-pyrazolo[3,4-c]quinolin-1-yl)phenyl)propanenitrile |
| 1168 | 2-methyl-2-(4-(8-(1-(methylsulfonyl)-1H-pyrazol-4-yl)-3H-pyrazolo[3,4-c]quinolin-1- |
| | yl)phenyl)propanenitrile |
| 1169 | 2-methyl-2-(4-(8-(1-phenyl-1H-pyrazol-4-yl)-3H-pyrazolo[3,4-c]quinolin-1-yl)phenyl)propanenitrile |
| 1170 | 2-methyl-2-(4-(8-(3-(4-phenylpiperazin-1-yl)phenyl)-3H-pyrazolo[3,4-c]quinolin-1- |
| | yl)phenyl)propanenitrile |
| 1180 | 2-methyl-2-(4-(8-(3-nitrophenyl)-3H-pyrazolo[3,4-c]quinolin-1-yl)phenyl)propanenitrile |
| 1181 | 2-methyl-2-(4-(8-(3-phenoxyphenyl)-3H-pyrazolo[3,4-c]quinolin-1-yl)phenyl)propanenitrile |
| 1182 | 2-methyl-2-(4-(8-(pyridazin-4-yl)-3H-pyrazolo[3,4-c]quinolin-1-yl)phenyl)propanenitrile |
| 1183 | 2-methyl-2-(4-(8-(1-(phenylsulfonyl)-1H-pyrazol-4-yl)-3H-pyrazolo[3,4-c]quinolin-1- |
| | yl)phenyl)propanenitrile |
| 1184 | 2-(4-(8-(1-benzyl-1H-pyrazol-4-yl)-3H-pyrazolo[3,4-c]quinolin-1-yl)phenyl)-2-methylpropanenitrile |
| 1185 | 2-(4-(8-(1H-indol-3-yl)-3H-pyrazolo[3,4-c]quinolin-1-yl)phenyl)-2-methylpropanenitrile |
| 1186 | 2-(4-(8-(1-isopropyl-1H-pyrazol-4-yl)-3H-pyrazolo[3,4-c]quinolin-1-yl)phenyl)-2- |
| | methylpropanenitrile |
| 1187 | 2-methyl-2-(4-(8-(1-(pyridin-4-ylmethyl)-1H-pyrazol-4-yl)-3H-pyrazolo[3,4-c]quinolin-1- |
| | yl)phenyl)propanenitrile |
| 1188 | 2-methyl-2-(4-(8-(pyridin-4-yl)-3H-pyrazolo[3,4-c]quinolin-1-yl)phenyl)propanenitrile |
| 1189 | N-(4-(1-(4-(2-cyanopropan-2-yl)phenyl)-3H-pyrazolo[3,4-c]quinolin-8-yl)phenyl)-N- |
| | methylacetamide |
| 1190 | N-(4-(1-(4-(2-cyanopropan-2-yl)phenyl)-3H-pyrazolo[3,4-c]quinolin-8-yl)phenyl)-N- |
| | methylmethanesulfonamide |
| 1191 | 4-(1-(4-(2-cyanopropan-2-yl)phenyl)-3H-pyrazolo[3,4-c]quinolin-8-yl)benzonitrile |
| 1192 | N-(4-(3-(1-(4-(2-cyanopropan-2-yl)phenyl)-3H-pyrazolo[3,4-c]quinolin-8- |
| | yl)phenylamino)phenyl)acetamide |
| 1193 | N-(4-(3-(1-(4-(2-cyanopropan-2-yl)phenyl)-3H-pyrazolo[3,4-c]quinolin-8- |
| | yl)phenylamino)phenyl)methanesulfonamide |
| 1194 | tert-butyl5-(1-(4-(2-cyanopropan-2-yl)phenyl)-3H-pyrazolo[3,4-c]quinolin-8-yl)pyridin-3- |
| | ylcarbamate |
| 1195 | 2-(4-(8-(4-(cyanomethyl)phenyl)-3H-pyrazolo[3,4-c]quinolin-1-yl)phenyl)-2-methylpropanenitrile |
| 1196 | 2-(4-(8-(4-(2-hydroxypropan-2-yl)phenyl)-3H-pyrazolo[3,4-c]quinolin-1-yl)phenyl)-2- |
| | methylpropanenitrile |
| 1200 | 2-methyl-2-(4-(8-(5-(4-methylpiperazin-1-yl)pyridin-3-yl)-3H-pyrazolo[3,4-c]quinolin-1- |
| | yl)phenyl)propanenitrile |
| 1201 | 2-methyl-2-(4-(8-(2-morpholinopyridin-4-yl)-3H-pyrazolo[3,4-c]quinolin-1-yl)phenyl)propanenitrile |
| 1202 | 5-(1-(4-(2-cyanopropan-2-yl)phenyl)-3H-pyrazolo[3,4-c]quinolin-8-yl)-N-methylpicolinamide |
| 1203 | N-(5-(1-(4-(2-cyanopropan-2-yl)phenyl)-3H-pyrazolo[3,4-c]quinolin-8-yl)pyridin-3- |
| | yl)methanesulfonamide |
| 1204 | N-(5-(1-(4-(2-cyanopropan-2-yl)phenyl)-3H-pyrazolo[3,4-c]quinolin-8-yl)pyridin-3- |
| | yl)cyclopropanecarboxamide cyclopropanecarboxylate salt |
| 1205 | 2-methyl-2-(4-(8-(2-(4-(methylsulfonyl)piperazin-1-yl)pyridin-4-yl)-3H-pyrazolo[3,4-c]quinolin-1- |
| | yl)phenyl)propanenitrile |
| 1206 | 2-(4-(8-(2-methoxyquinolin-6-yl)-3H-pyrazolo[3,4-c]quinolin-1-yl)phenyl)-2-methylpropanenitrile |
| 1207 | 5-(1-(4-(2-cyanopropan-2-yl)phenyl)-3H-pyrazolo[3,4-c]quinolin-8-yl)-N,N-dimethylpicolinamide |
| 1208 | 2-(4-(8-(2-(4-acetylpiperazin-1-yl)pyridin-4-yl)-3H-pyrazolo[3,4-c]quinolin-1-yl)phenyl)-2- |
| | methylpropanenitrile |
| 1209 | 2-methyl-2-(4-(8-(6-(morpholinomethyl)pyridin-3-yl)-3H-pyrazolo[3,4-c]quinolin-1- |
| | yl)phenyl)propanenitrile |
| 1210 | 2-(4-(8-(5-(isopropylamino)pyridin-3-yl)-3H-pyrazolo[3,4-c]quinolin-1-yl)phenyl)-2- |
| | methylpropanenitrile |
| 1211 | 2-(4-(8-(5-(4-acetylpiperazin-1-yl)pyridin-3-yl)-3H-pyrazolo[3,4-c]quinolin-1-yl)phenyl)-2- |
| | methylpropanenitrile |
| 1212 | N-(5-(1-(4-(2-cyanopropan-2-yl)phenyl)-3H-pyrazolo[3,4-c]quinolin-8-yl)pyridin-2- |
| | yl)methanesulfonamide |
| 1213 | N-(5-(1-(4-(2-cyanopropan-2-yl)phenyl)-3H-pyrazolo[3,4-c]quinolin-8-yl)pyridin-2- |
| | yl)cyclopropanecarboxamide cyclopropanecarboxylate |
| 1214 | 5-(1-(4-(2-cyanopropan-2-yl)phenyl)-3H-pyrazolo[3,4-c]quinolin-8-yl)-N,N-dimethylnicotinamide |
| 1215 | N-benzyl-5-(1-(4-(2-cyanopropan-2-yl)phenyl)-3H-pyrazolo[3,4-c]quinolin-8-yl)picolinamide |
| 1216 | N-(5-(1-(4-(2-cyanopropan-2-yl)phenyl)-3H-pyrazolo[3,4-c]quinolin-8-yl)pyridin-2-yl)benzamide |
| 1217 | 2-(4-(8-(5-(1H-imidazol-1-yl)pyridin-3-yl)-3H-pyrazolo[3,4-c]quinolin-1-yl)phenyl)-2- |
| | methylpropanenitrile |
| 1218 | 2-methyl-2-(4-(8-(5-(oxazol-2-yl)pyridin-3-yl)-3H-pyrazolo[3,4-c]quinolin-1- |
| | yl)phenyl)propanenitrile |
| 1219 | 2-methyl-2-(4-(8-(6-(morpholine-4-carbonyl)pyridin-3-yl)-3H-pyrazolo[3,4-c]quinolin-1- |
| | yl)phenyl)propanenitrile |
| 1220 | 2-(4-(8-(6-ethoxypyridin-3-yl)-3H-pyrazolo[3,4-c]quinolin-1-yl)phenyl)-2-methylpropanenitrile |
| 1221 | 2-methyl-2-(4-(8-(6-morpholinopyridin-3-yl)-3H-pyrazolo[3,4-c]quinolin-1-yl)phenyl)propanenitrile |
| 1222 | 2-methyl-2-(4-(8-(6-(pyrrolidin-1-yl)pyridin-3-yl)-3H-pyrazolo[3,4-c]quinolin-1- |
| | yl)phenyl)propanenitrile |
| 1223 | N-benzyl-5-(1-(4-(2-cyanopropan-2-yl)phenyl)-3H-pyrazolo[3,4-c]quinolin-8-yl)nicotinamide |
| 1224 | 2-methyl-2-(4-(8-(5-(morpholinomethyl)pyridin-3-yl)-3H-pyrazolo[3,4-c]quinolin-1- |
| | yl)phenyl)propanenitrile |
| 1225 | 2-methyl-2-(4-(8-(6-(oxazol-2-yl)pyridin-3-yl)-3H-pyrazolo[3,4-c]quinolin-1- |
| | yl)phenyl)propanenitrile |
| 1226 | 2-methyl-2-(4-(8-(5-(pyrrolidin-1-yl)pyridin-3-yl)-3H-pyrazolo[3,4-c]quinolin-1- |
| | yl)phenyl)propanenitrile |
| 1227 | 5-(1-(4-(2-cyanopropan-2-yl)phenyl)-3H-pyrazolo[3,4-c]quinolin-8-yl)-N-isopropylnicotinamide |
| 1228 | 2-methyl-2-(4-(8-(6-(2-oxopyrrolidin-1-yl)pyridin-3-yl)-3H-pyrazolo[3,4-c]quinolin-1- |
| | yl)phenyl)propanenitrile |
| 1229 | 5-(1-(4-(2-cyanopropan-2-yl)phenyl)-3H-pyrazolo[3,4-c]quinolin-8-yl)picolinonitrile |
| 1230 | 2-(4-(8-(6-aminopyridin-3-yl)-3H-pyrazolo[3,4-c]quinolin-1-yl)phenyl)-2-methylpropanenitrile |
| 1231 | 2-(4-(8-(6-hydroxypyridin-3-yl)-3H-pyrazolo[3,4-c]quinolin-1-yl)phenyl)-2-methylpropanenitrile |
| 1232 | 5-(1-(4-(2-cyanopropan-2-yl)phenyl)-3H-pyrazolo[3,4-c]quinolin-8-yl)-N-cyclopropylnicotinamide |
| 1233 | 2-(4-(8-(9H-carbazol-2-yl)-3H-pyrazolo[3,4-c]quinolin-1-yl)phenyl)-2-methylpropanenitrile |
| 1234 | 6-(1-(4-(2-cyanopropan-2-yl)phenyl)-3H-pyrazolo[3,4-c]quinolin-8-yl)picolinonitrile |
| 1235 | 2-methyl-2-(4-(8-(3-morpholinophenyl)-3H-pyrazolo[3,4-c]quinolin-1-yl)phenyl)propanenitrile |
| 1236 | 2-(4-(8-(3-(4-acetylpiperazin-1-yl)phenyl)-3H-pyrazolo[3,4-c]quinolin-1-yl)phenyl)-2- |
| | methylpropanenitrile |
| 1237 | 2-methyl-2-(4-(8-(3-(4-(methylsulfonyl)piperazin-1-yl)phenyl)-3H-pyrazolo[3,4-c]quinolin-1- |
| | yl)phenyl)propanenitrile |
| 1238 | 2-methyl-2-(4-(8-(thieno[2,3-b]pyridin-2-yl)-3H-pyrazolo[3,4-c]quinolin-1-yl)phenyl)propanenitrile |
| 1239 | 2-methyl-2-(4-(8-(3-(pyridin-4-yloxy)phenyl)-3H-pyrazolo[3,4-c]quinolin-1-yl)phenyl)propanenitrile |
| 1240 | 3-(1-(4-(2-cyanopropan-2-yl)phenyl)-3H-pyrazolo[3,4-c)quinolin-8-yl)-N,N-dimethylbenzamide |
| 1241 | 2-(4-(8-(3-(4-fluorophenylamino)phenyl)-3H-pyrazolo[3,4-c]quinolin-1-yl)phenyl)-2- |
| | methylpropanenitrile |
| 1242 | N-benzyl-5-(1-(4-(2-cyanopropan-2-yl)phenyl)-3H-pyrazolo[3,4-c]quinolin-8-yl)picolinamide |
| 1243 | 2-methyl-2-(4-(8-(6-(morpholine-4-carbonyl)pyridin-3-yl)-3H-pyrazolo[3,4-c]quinolin-1- |
| | yl)phenyl)propanenitrile |
| 1244 | 2-methyl-2-(4-(8-(3-(4-methylpiperazin-1-yl)phenyl)-3H-pyrazolo[3,4-c]quinolin-1- |
| | yl)phenyl)propanenitrile |
| 1245 | 2-(4-(8-(1H-indazol-6-yl)-3H-pyrazolo[3,4-c]quinolin-1-yl)phenyl)-2-methylpropanenitrile |
| 1246 | 2-(4-(8-(dibenzo[b,d]furan-3-yl)-3H-pyrazolo[3,4-c]quinolin-1-yl)phenyl)-2-methylpropanenitrile |
| 1247 | 2-(4-(8-(1H-indazol-3-yl)-3H-pyrazolo[3,4-c]quinolin-1-yl)phenyl)-2-methylpropanenitrile |
| 1248 | 2-methyl-2-(4-(8-(pyridin-2-yl)-3H-pyrazolo[3,4-c]quinolin-1-yl)phenyl)propanenitrile |
| 1249 | 2-(4-(8-(6-methoxypyridin-2-yl)-3H-pyrazolo[3,4-c]quinolin-1-yl)phenyl)-2-methylpropanenitrile |
| 1250 | 2-(4-(8-(6-chloro-4-methylpyridin-3-yl)-3H-pyrazolo[3,4-c]quinolin-1-yl)phenyl)-2- |
| | methylpropanenitrile |
| 1251 | 2-(4-(8-(6-chlorothieno[2,3-b]pyridin-2-yl)-3H-pyrazolo[3,4-c]quinolin-1-yl)phenyl)-2- |
| | methylpropanenitrile |
| 1252 | N-(5-(1-(4-(2-cyanopropan-2-yl)phenyl)-3H-pyrazolo[3,4-c]quinolin-8-yl)pyridin-2-yl)acetamide |
| 1253 | 2-methyl-2-(4-(8-(6-phenoxypyridin-3-yl)-3H-pyrazolo[3,4-c]quinolin-1-yl)phenyl)propanenitrile |
| 1254 | 2-(4-(8-(3-aminophenyl)-3H-pyrazolo[3,4-c]quinolin-1-yl)phenyl)-2-methylpropanenitrile |
| 1255 | N-(3-(1-(4-(2-cyanopropan-2-yl)phenyl)-3H-pyrazolo[3,4-c]quinolin-8-yl)phenyl)benzamide |
| 1256 | N-(3-(1-(4-(2-cyanopropan-2-yl)phenyl)-3H-pyrazolo[3,4-c]quinolin-8- |
| | yl)phenyl)benzenesulfonamide |
| 1257 | N-(3-(1-(4-(2-cyanopropan-2-yl)phenyl)-3H-pyrazolo[3,4-c]quinolin-8-yl)phenyl)acetamide |
| 1258 | N-(3-(1-(4-(2-cyanopropan-2-yl)phenyl)-3H-pyrazolo[3,4-c]quinolin-8- |
| | yl)phenyl)methanesulfonamide |
| 1259 | 2-methyl-2-(4-(8-(3-(4-nitrophenylamino)phenyl)-3H-pyrazolo[3,4-c]quinolin-1- |
| | yl)phenyl)propanenitrile |
| 1260 | 2-(4-(8-(3-(4-aminophenylamino)phenyl)-3H-pyrazolo[3,4-c]quinolin-1-yl)phenyl)-2- |
| | methylpropanenitrile |
| 1261 | N-(5-(1-(4-(2-cyanopropan-2-yl)phenyl)-3H-pyrazolo[3,4-c]quinolin-8-yl)pyridin-3- |
| | yl)benzenesulfonamide |
| 1262 | 2-(4-(8-(5-aminopyridin-3-yl)-3H-pyrazolo[3,4-c]quinolin-1-yl)phenyl)-2-methylpropanenitrile |
| 1263 | N-(5-(1-(4-(2-cyanopropan-2-yl)phenyl)-3H-pyrazolo[3,4-c]quinolin-8-yl)pyridin-3-yl)benzamide |
| 1264 | N-(5-(1-(4-(2-cyanopropan-2-yl)phenyl)-3H-pyrazolo[3,4-c]quinolin-8-yl)pyridin-3-yl)acetamide |
| 1265 | N-(5-(1-(4-(2-cyanopropan-2-yl)phenyl)-3H-pyrazolo[3,4-c]quinolin-8-yl)pyridin-3- |
| | yl)methanesulfonamide |
| 1266 | N-(5-(1-(4-(2-cyanopropan-2-yl)phenyl)-3H-pyrazolo[3,4-c]quinolin-8-yl)pyridin-3- |
| | yl)methanesulfonamide |
| 1267 | N-(5-(1-(4-(2-cyanopropan-2-yl)phenyl)-3H-pyrazolo[3,4-c]quinolin-8-yl)pyridin-3- |
| | yl)cyclopropanecarboxamide cyclopropanecarboxylate salt |
| 1268 | 2-methyl-2-(4-(8-(4-methylpiperazin-1-yl)-3H-pyrazolo[3,4-c]quinolin-1-yl)phenyl)propanenitrile |
| 1269 | 2-(4-(8-(dimethylamino)-3H-pyrazolo[3,4-c]quinolin-1-yl)phenyl)-2-methylpropanenitrile |
| 1270 | 2-methyl-2-(4-(8-morpholino-3H-pyrazolo[3,4-c]quinolin-1-yl)phenyl)propanenitrile |
| 1271 | 2-methyl-2-(4-(8-(4-(methylsulfonyl)piperazin-1-yl)-3H-pyrazolo[3,4-c]quinolin-1- |
| | yl)phenyl)propanenitrile |
| 1272 | 2-(4-(8-hydroxy-3H-pyrazolo[3,4-c]quinolin-1-yl)phenyl)-2-methylpropanenitrile |
| 1273 | 2-methyl-2-(4-(2-methyl-8-(pyridin-3-yl)-2H-pyrazolo[3,4-c]quinolin-1-yl)phenyl)propanenitrile |
| 1274 | 2-methyl-2-(4-(3-methyl-8-(pyridin-3-yl)-3H-pyrazolo[3,4-c]quinolin-1-yl)phenyl)propanenitrile |
| 1275 | 2-(4-(2-ethyl-8-(pyridin-3-yl)-2H-pyrazolo[3,4-c]quinolin-1-yl)phenyl)-2-methylpropanenitrile |
| 1276 | 2-(4-(3-ethyl-8-(pyridin-3-yl)-3H-pyrazolo[3,4-c]quinolin-1-yl)phenyl)-2-methylpropanenitrile |
| 1277 | 2-(4-(3-allyl-8-(pyridin-3-yl)-3H-pyrazolo[3,4-c]quinolin-1-yl)phenyl)-2-methylpropanenitrile |
| 1278 | 2-(4-(8-(1H-indol-3-yl)-3-methyl-3H-pyrazolo[3,4-c]quinolin-1-yl)phenyl)-2-methylpropanenitrile |
| 1279 | 2-methyl-2-(4-(3-methyl-8-(quinolin-3-yl)-3H-pyrazolo[3,4-c]quinolin-1-yl)phenyl)propanenitrile |
| 1280 | N-(3-(1-(4-(2-cyanopropan-2-yl)phenyl)-3-methyl-3H-pyrazolo[3,4-c]quinolin-8- |
| | yl)phenyl)methanesulfonamide |
| 1281 | 2-methyl-2-(4-(3-methyl-8-(quinolin-7-yl)-3H-pyrazolo[3,4-c]quinolin-1-yl)phenyl)propanenitrile |
| 1282 | 2-methyl-2-(4-(3-methyl-8-(3-morpholinophenyl)-3H-pyrazolo[3,4-c]quinolin-1- |
| | yl)phenyl)propanenitrile |
| 1283 | 2-methyl-2-(4-(3-methyl-8-(3-(pyridin-4-ylamino)phenyl)-3H-pyrazolo[3,4-c]quinolin-1- |
| | yl)phenyl)propanenitrile |
| 1284 | N-(4-(1-(4-(2-cyanopropan-2-yl)phenyl)-3-methyl-3H-pyrazolo[3,4-c]quinolin-8-yl)phenyl)-N- |
| | methylacetamide |
| 1285 | N-(4-(1-(4-(2-cyanopropan-2-yl)phenyl)-3-methyl-3H-pyrazolo[3,4-c]quinolin-8-yl)phenyl)-N- |
| | methylmethanesulfonamide |
| 1286 | 2-(4-(8-(3-(4-acetylpiperazin-1-yl)phenyl)-3-methyl-3H-pyrazolo[3,4-c]quinolin-1-yl)phenyl)-2- |
| | methylpropanenitrile |
| 1287 | 2-methyl-2-(4-(3-methyl-8-(3-(4-(methylsulfonyl)piperazin-1-yl)phenyl)-3H-pyrazolo[3,4- |
| | c]quinolin-1-yl)phenyl)propanenitrile |
| 1288 | 2-(4-(8-(3-(4-benzylpiperazin-1-yl)phenyl)-3-methyl-3H-pyrazolo[3,4-c]quinolin-1-yl)phenyl)-2- |
| | methylpropanenitrile |
| 1289 | tert-butyl5-(1-(4-(2-cyanopropan-2-yl)phenyl)-3-methyl-3H-pyrazolo[3,4-c]quinolin-8-yl)pyridin-2- |
| | ylcarbamate |
| 1290 | 2-(4-(8-(5-aminopyridin-3-yl)-3-methyl-3H-pyrazolo[3,4-c]quinolin-1-yl)phenyl)-2- |
| | methylpropanenitrile |
| 1291 | N-(5-(1-(4-(2-cyanopropan-2-yl)phenyl)-3-methyl-3H-pyrazolo[3,4-c]quinolin-8-yl)pyridin-3- |
| | yl)acetamide |
| 1292 | N-(5-(1-(4-(2-cyanopropan-2-yl)phenyl)-3-methyl-3H-pyrazolo[3,4-c]quinolin-8-yl)pyridin-3- |
| | yl)methanesulfonamide |
| 1293 | N-(5-(1-(4-(2-cyanopropan-2-yl)phenyl)-3-methyl-3H-pyrazolo[3,4-c]quinolin-8-yl)pyridin-2- |
| | yl)acetamide |
| 1294 | N-(5-(1-(4-(2-cyanopropan-2-yl)phenyl)-3-methyl-3H-pyrazolo[3,4-c]quinolin-8-yl)pyridin-2- |
| | yl)methanesulfonamide |
| 1295 | 2-methyl-2-(4-(3-methyl-8-(5-morpholinopyridin-3-yl)-3H-pyrazolo[3,4-c]quinolin-1- |
| | yl)phenyl)propanenitrile |
| 1296 | 2-methyl-2-(4-(3-methyl-8-(5-(4-methylpiperazin-1-yl)pyridin-3-yl)-3H-pyrazolo[3,4-c]quinolin-1- |
| | yl)phenyl)propanenitrile |
| 1297 | 5-(1-(4-(2-cyanopropan-2-yl)phenyl)-3-methyl-3H-pyrazolo[3,4-c]quinolin-8-yl)-N- |
| | cyclopropylpicolinamide |
| 1298 | 5-(1-(4-(2-cyanopropan-2-yl)phenyl)-3-methyl-3H-pyrazolo[3,4-c]quinolin-8-yl)-N- |
| | cyclopropylnicotinamide |
| 1299 | 2-(4-(8-(5-(dimethylamino)pyridin-3-yl)-3-methyl-3H-pyrazolo[3,4-c]quinolin-1-yl)phenyl)-2- |
| | methylpropanenitrile |
| 1300 | 5-(1-(4-(2-cyanopropan-2-yl)phenyl)-3-methyl-3H-pyrazolo[3,4-c]quinolin-8-yl)-N- |
| | methylnicotinamide |
| 1301 | 2-(4-(8-(5-(isopropylamino)pyridin-3-yl)-3-methyl-3H-pyrazolo[3,4-c]quinolin-1-yl)phenyl)-2- |
| | methylpropanenitrile |
| 1302 | 2-(4-(8-(5-hydroxypyridin-3-yl)-3-methyl-3H-pyrazolo[3,4-c]quinolin-1-yl)phenyl)-2- |
| | methylpropanenitrile |
| 1303 | N-(5-(1-(4-(2-cyanopropan-2-yl)phenyl)-3-methyl-3H-pyrazolo[3,4-c]quinolin-8-yl)pyridin-3- |
| | yl)cyclopropanecarboxamide |
| 1304 | 5-(1-(4-(2-cyanopropan-2-yl)phenyl)-3-methyl-3H-pyrazolo[3,4-c]quinolin-8-yl)-N- |
| | methylpicolinamide |
| 1305 | 8-(pyridin-3-yl)-3H-pyrazolo[3,4-c]quinoline |
| 1306 | methyl 2-(8-(pyridin-3-yl)-3H-pyrazolo[3,4-c]quinolin-3-yl)acetate |
| 1307 | 3-methyl-8-(pyridin-3-yl)-3H-pyrazolo[3,4-c]quinoline |
| 1308 | 2-methyl-8-(pyridin-3-yl)-2H-pyrazolo[3,4-c]quinoline |
| 1309 | 2-methyl-8-(pyridin-3-yl)-2H-pyrazolo[3,4-c]quinoline |
| 1310 | 2-ethyl-8-(pyridin-3-yl)-2H-pyrazolo[3,4-c]quinoline |
| 1311 | N-methyl-N-(4-(8-(pyridin-3-yl)-3H-pyrazolo[3,4-c]quinolin-1-yl)phenyl)acetamide |
| 1312 | 4-(8-(pyridin-3-yl)-3H-pyrazolo[3,4-c]quinolin-1-yl)benzonitrile |
| 1313 | 2-(4-(8-(pyridin-3-yl)-3H-pyrazolo[3,4-c]quinolin-1-yl)phenyl)propan-2-ol |
| 1314 | 2-(4-(8-(pyridin-3-yl)-3H-pyrazolo[3,4-c]quinolin-1-yl)phenyl)acetonitrile |
| 1315 | 1,8-di(pyridin-3-yl)-3H-pyrazolo[3,4-c]quinoline |
| 1316 | tert-butyl 5-(8-(pyridin-3-yl)-3H-pyrazolo[3,4-c]quinolin-1-yl)pyridin-3-ylcarbamate |
| 1317 | N-(4-(8-(pyridin-3-yl)-3H-pyrazolo[3,4-c]quinolin-1-yl)phenyl)methanesulfonamide |
| 1318 | 1-(4-(8-(pyridin-3-yl)-3H-pyrazolo[3,4-c]quinolin-1-yl)phenyl)pyrrolidin-2-one |
| 1319 | N-methyl-N-(4-(8-(pyridin-3-yl)-3H-pyrazolo[3,4-c]quinolin-1-yl)phenyl)methanesulfonamide |
| 1320 | 8-bromo-3H-pyrazolo[3,4-c]quinoline |
| 1321 | N-(5-(3H-pyrazolo[3,4-c]quinolin-8-yl)pyridin-3-yl)acetamide |
| 1322 | N-(5-(3-benzyl-3H-pyrazolo[3,4-c]quinolin-8-yl)pyridin-3-yl)acetamide |
| 1323 | N-(5-(3-benzyl-1-bromo-3H-pyrazolo[3,4-c]quinolin-8-yl)pyridin-3-yl)acetamide |
| 1324 | N-(5-(2-benzyl-2H-pyrazolo[3,4-c]quinolin-8-yl)pyridin-3-yl)acetamide |
| 1325 | N-(5-(3-benzyl-1-(4-cyanophenyl)-3H-pyrazolo[3,4-c]quinolin-8-yl)pyridin-3-yl)acetamide |
| 1326 | 3-benzyl-8-bromo-1-morpholino-3H-pyrazolo[3,4-c]quinoline |
| 1327 | N-(5-(1-(pyridin-4-yl)-3H-pyrazolo[3,4-c]quinolin-8-yl)pyridin-3-yl)acetamide |
| 1328 | 3-benzyl-1,8-dimorpholino-3H-pyrazolo[3,4-c]quinoline |
| 1329 | 1,8-dimorpholino-3H-pyrazolo[3,4-c]quinoline |
| 1330 | 2-methyl-2-(4-(3-oxo-9-(pyridin-3-yl)-3,4-dihydropyrimido[4,5-c]quinolin-1- |
| | yl)phenyl)propanenitrile |
| 1331 | 2-methyl-2-(4-(9-(pyridin-3-yl)pyrimido[4,5-c]quinolin-1-yl)phenyl)propanenitrile |
| 1332 | 2-methyl-2-(4-(8-(pyridin-3-yl)isoxazolo[5,4-c]quinolin-1-yl)phenyl)propanenitrile |
| 1333 | 2-methyl-2-(4-(8-(pyridin-3-yl)isoxazolo[3,4-c]quinolin-1-yl)phenyl)propanenitrile |
| 1334 | 2-methyl-2-(4-(8-(pyridin-3-yl)-1H-[1,2,3]triazolo[4,5-c]quinolin-1-yl)phenyl)propanenitrile |
| 1335 | 2-methyl-2-(4-(8-(pyridin-3-yl)isothiazolo[3,4-c]quinolin-1-yl)phenyl)propanenitrile |
| 1336 | 2-methyl-2-(4-(8-(pyridin-3-yl)isothiazolo[5,4-c]quinolin-1-yl)phenyl)propanenitrile |
| 1337 | N-benzyl-5-(1-(4-(2-cyanopropan-2-yl)phenyl)-3-methyl-3H-pyrazolo[3,4-c]quinolin-8- |
| | yl)nicotinamide |
| 1338 | 2-methyl-2-(4-(3-methyl-8-(2-(4-methylpiperazin-1-yl)pyridin-4-yl)-3H-pyrazolo[3,4-c]quinolin-1- |
| | yl)phenyl)propanenitrile |
| 1339 | 2-(4-(8-(6-(dimethylamino)pyridin-3-yl)-3H-pyrazolo[3,4-c]quinolin-1-yl)phenyl)-2- |
| | methylpropanenitrile |
| 1340 | 2-(4-(8-(6-(dimethylamino)pyridin-3-yl)-3-methyl-3H-pyrazolo[3,4-c]quinolin-1-yl)phenyl)-2- |
| | methylpropanenitrile |
| 1341 | 2-methyl-2-(4-(3-methyl-8-(6-morpholinopyridin-3-yl)-3H-pyrazolo[3,4-c]quinolin-1- |
| | yl)phenyl)propanenitrile |
| 1342 | 3-benzyl-8-(6-ethoxypyridin-3-yl)-1-morpholino-3H-pyrazolo[3,4-c]quinoline |
| 1343 | 8-(6-ethoxypyridin-3-yl)-1-morpholino-3H-pyrazolo[3,4-c]quinoline |
| 1344 | 2-(4-(8-(5-methoxypyridin-3-yl)-3H-pyrazolo[3,4-c]quinolin-1-yl)phenyl)-2-methylpropanenitrile |
| 1345 | 3-benzyl-1-(4-methylpiperazin-1-yl)-3H-pyrazolo[3,4-c]quinoline |
| 1346 | 1-(4-methylpiperazin-1-yl)-3H-pyrazolo[3,4-c]quinoline |
| 1347 | N-(5-(1-(4-cyanophenyl)-3H-pyrazolo[3,4-c]quinolin-8-yl)pyridin-3-yl)acetamide |
| 1348 | 5-(1-(4-(2-cyanopropan-2-yl)phenyl)-3-methyl-3H-pyrazolo[3,4-c]quinolin-8-yl)picolinamide |
| 1349 | N-(5-(1-(4-(2-cyanopropan-2-yl)phenyl)-2-methyl-2H-pyrazolo[3,4-c]quinolin-8-yl)pyridin-3- |
| | yl)cyclopropanecarboxamide |
| 1350 | 2-methyl-2-(4-(2-methyl-8-(5-morpholinopyridin-3-yl)-2H-pyrazolo[3,4-c]quinolin-1- |
| | yl)phenyl)propanenitrile |
| 1351 | N-(4-(1-(4-(2-cyanopropan-2-yl)phenyl)-3-methyl-3H-pyrazolo[3,4-c]quinolin-8-yl)pyridin-2- |
| | yl)acetamide |
| 1352 | 2-methyl-2-(4-(3-methyl-8-(6-(oxazol-2-yl)pyridin-3-yl)-3H-pyrazolo[3,4-c]quinolin-1- |
| | yl)phenyl)propanenitrile |
| 1353 | 2-(4-(8-(6-(1H-pyrazol-1-yl)pyridin-3-yl)-3-methyl-3H-pyrazolo[3,4-c]quinolin-1-yl)phenyl)-2- |
| | methylpropanenitrile |
| 1354 | N-(5-(3-benzyl-1-(4-(2-cyanopropan-2-yl)phenyl)-3H-pyrazolo[3,4-c]quinolin-8-yl)pyridin-3- |
| | yl)acetamide |
| 1355 | N-(5-(3-benzyl-1-(4-(2-cyanopropan-2-yl)phenyl)-3H-pyrazolo[3,4-c]quinolin-8- |
| | yl)pyridin-3-yl)methanesulfonamide |
| 1356 | N-(5-(2-benzyl-1-(4-(2-cyanopropan-2-yl)phenyl)-2H-pyrazolo[3,4-c]quinolin-8-yl)pyridin-3- |
| | yl)acetamide |
| 1357 | N-(5-(2-benzyl-1-(4-(2-cyanopropan-2-yl)phenyl)-2H-pyrazolo[3,4-c]quinolin-8-yl)pyridin-3- |
| | yl)methanesulfonamide |
| 1358 | 5-(1-(4-(2-cyanopropan-2-yl)phenyl)-3-methyl-3H-pyrazolo[3,4-c]quinolin-8-yl)nicotinonitrile |
| 1359 | 5-(1-(4-(2-cyanopropan-2-yl)phenyl)-3H-pyrazolo[3,4-c]quinolin-8-yl)nicotinonitrile |
| 1360 | 2-(4-(8-(6-hydroxypyridin-3-yl)-3-methyl-3H-pyrazolo[3,4-c]quinolin-1-yl)phenyl)-2- |
| | methylpropanenitrile |
| 1361 | 3-benzyl-8-bromo-1-(4-methylpiperazin-1-yl)-3H-pyrazolo[3,4-c]quinoline |
| 1362 | 2-(4-(8-(5-(1H-imidazol-1-yl)pyridin-3-yl)-3-methyl-3H-pyrazolo[3,4-c]quinolin-1-yl)phenyl)-2- |
| | methylpropanenitrile |
| 1363 | 2-(4-(8-(2-methoxypyridin-4-yl)-3-methyl-3H-pyrazolo[3,4-c]quinolin-1-yl)phenyl)-2- |
| | methylpropanenitrile |
| 1364 | N-(5-(1-(4-acetamidophenyl)-3H-pyrazolo[3,4-c]quinolin-8-yl)pyridin-3-yl)acetamide |
| 1365 | 1-(2-benzyl-2H-indazol-4-yl)-6-((4-(methylsulfonyl)piperazin-1-yl)methyl)-3-morpholino-1H- |
| | indazole |
| 1366 | 2-methyl-2-(4-(3-methyl-8-(6-methylpyridin-3-yl)-3H-pyrazolo[3,4-c]quinolin-1- |
| | yl)phenyl)propanenitrile |
| 1367 | 2-(4-(8-(2-aminopyridin-4-yl)-3H-pyrazolo[3,4-c]quinolin-1-yl)phenyl)-2-methylpropanenitrile |
| 1368 | 2-(4-(8-(6-(1H-imidazol-1-yl)pyridin-3-yl)-3-methyl-3H-pyrazolo[3,4-c]quinolin-1-yl)phenyl)-2- |
| | methylpropanenitrile |
| 1369 | 5-(1-(4-(2-cyanopropan-2-yl)phenyl)-3H-pyrazolo[3,4-c]quinolin-8-yl)picolinamide |
| 1370 | 1-(1H-indazol-4-yl)-6-((4-(methylsulfonyl)piperazin-1-yl)methyl)-3-morpholino-1H-indazole |
| 1371 | 1-(2-benzyl-2H-indazol-4-yl)-5-((4-(methylsulfonyl)piperazin-1-yl)methyl)-3-morpholino-1H-indazole |
| 1372 | 1-(1H-indazol-4-yl)-5-((4-(methylsulfonyl)piperazin-1-yl)methyl)-3-morpholino-1H-indazole |
| 1373 | 2-(4-(8-(1-benzyl-1H-pyrazol-4-yl)-3-methyl-3H-pyrazolo[3,4-c]quinolin-1-yl)phenyl)-2- |
| | methylpropanenitrile |

In an embodiment of the invention, synthetic methods of preparing a compound of the invention are provided in the examples described below. In particular, a method is provided for stereoselective synthesis of a salt form of a novel compound as described above. For example, the halide salt form may be bromide, iodide, chloride, or fluoride. The organic anionic-charged species can be, for example, a sulfonate or carboxylate. Exemplary sulfonates are mesylate, besylate, tosylate, or triflate. Exemplary carboxylates are formate, acetate, citrate, or fumarate. The method can further involve exchanging an anion with a different anion. The alkylating agent can be an alkyl group susceptible to nucleophilic attack, and a leaving group. Exemplary methylating agents may be selected from the group consisting of methyl halide, dimethyl sulfate, methyl nitrate and methyl sulfonate. Methyl halides are methyl iodide, methyl bromide, methyl chloride and methyl fluoride. Methyl sulfonates include methyl mesylate, methyl besylate, methyl tosylate, and methyl triflate. In one embodiment, the alkylation is conducted at a temperature range of about 70° C. to about 100° C., or of about 80° C. to about 90° C., or at a temperature of about 88° C. The alkylation reaction may be conducted for a significant period of time, for example, about 1 hour to 24 hours, or about 5 hour to 16 hours or for about 10 hours. The method can further involve purification of the salt using at least one purification technique, such as chromatography or recrystallization. The chromatography can be reverse-phase chromatography or regular phase chromatography. In some embodiments, the regular phase chromatography can use alumina or silica gel. The intermediate can be purified prior to alkylation. According to another embodiment of the invention, a method for isolation and purification of the novel compounds is provided, comprising passing the crude reaction products through a chromatography column and collecting the particular compound which elutes at the appropriate retention time. This process can be used in addition to the method described above, after the deprotecting step and/or the anion exchange resin column step. A novel PI3K kinase and/or protein kinase inhibitor compound of the invention may also be isolated by similar methods.

According to another embodiment of the invention, a method for analyzing stereoisomers is provided. The method involves conducting high performance liquid chromatography (HPLC) and applying specific compound of according to Formula (I-IV) to the chromatography column as a standard. The method preferably involves applying both types of stereoisomers as standards to determine relative retention/elution times.

The foregoing HPLC can be used to determine the relative amount of stereoisomer and the intermediates of the synthesis thereof by determining the area under the respective curves in the chromatogram produced. According to another aspect of the invention a method for isolation and purification of salt intermediate is provided, comprising recrystallizing the crude products or intermediates thereof from a solvent or a mixture of solvents. This process can be in addition to the method described above, after the deprotection step and/or the anion exchange resin column step.

The pharmaceutical preparations of the invention embrace a variety of forms, including, but not limited to, a composition that is enteric coated, a composition that is a controlled release or sustained release formulation, a composition that is a solution, a composition that is a topical formulation, a composition that is a suppository, a composition that is a transdermal patch, a composition that is lyophilized, a composition that is in an inhaler, a compositions that is in a prefilled syringe, a composition that is in a nasal spray device, and the like. The composition can be for oral administration, parenteral administration, mucosal administration, nasal administration, topical administration, ocular administration, local administration, rectal, intrathecal, etc. If parenteral, the administration can be subcutaneous, intravenous, intradermal, intraperitoneal, intrathecal, etc. The pharmaceutical preparation may be in a packaged unit dosage or multi-unit dosage. Routes of administration of the compounds in a pharmaceutically acceptable form may include, without limitation, parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

According to yet another embodiment of the invention, a pharmaceutical preparation containing a compound of the present invention, prodrug, salt or intermediate, in a lyophilized formulation is prepared by combining a cryoprotective agent, such as mannitol, with the same. The lyophilized preparation may also contain any one of, any combination of, or all of a buffering agent, an antioxidant, and an isotonicity agent. In yet another embodiment, the pharmaceutical composition can further comprise at least one compound of the invention, and at least one additional pharmaceutical agent, for example, an agent that is not a PI3K inhibitor. In various embodiments, the pharmaceutical agent is an antiviral agent, an anti-infective agent, an anticancer agent, an antispasmodic agent, an anti-muscarinic agent, a steroidal or non-steroidal anti-inflammatory agent, a pro-motility agent, a 5HT₁ agonist, a 5HT₃ antagonist, a 5HT₄ antagonist, a 5HT₄ agonist, a bile salt sequestering agent, a bulk-forming agent, an alpha2-adrenergic agonist, a mineral oil, an antidepressant, a herbal medicine, an anti-diarrheal medication, a laxative, a stool softener, a fiber or a hematopoietic stimulating agent.

More particularly, depending on the disease or condition to be treated or prevented, one or more additional therapeutic drugs, compounds, reagents, or agents, which are normally or typically administered to treat or prevent the disease or condition, may also be administered with the compounds of this invention, or may also be present in the compositions of this invention. It will be appreciated that additional therapeutic agents that are normally or typically administered to treat or prevent a given disease or condition are termed “appropriate for the disease or condition being treated”.

In an embodiment, chemotherapeutic agents or other antiproliferative agents may be co-administered, administered together with (either at the same time or a different time), or combined with the compounds of the present invention to treat proliferative diseases, tumors, or cancers. Illustrative yet nonlimiting chemotherapeutic drugs that are suitable include alkylating drugs, e.g., cyclophosphamide, melphalan, mechlorethamine, chlorambucil, Ifosfamide; antimetabolites, e.g., methotrexate; purine antagonists and pyrimidine antagonists, e.g., 6-mercaptopurine, 5-fluorouracil, fluorouracil, cytarabile, gemcitabine; spindle poisons, e.g., vinblastine, vincristine, vinorelbine, paclitaxel; podophyllotoxins, e.g., etoposide, irinotecan, topotecan; antibiotics, e.g., doxorubicin, bleomycin, mitomycin, adriamycin, dexamethasone; nitrosoureas, e.g., Carmustine, Lomustine; inorganic ions, e.g., cisplatin, carboplatin; enzymes, e.g., asparaginase; biologic response modifiers, e.g., interleukins, tumor suppressor factors, interleukins, tumor necrosis factor (TNF), hormones, e.g., Tamoxifen, Leuprolide, Flutamide, Megestrol; small molecule inhibitor drugs, e.g., Gleevec®, Sutent®; cyclophosphamide, Taxol, and platinum derivatives.

In another embodiment, other agents, compounds, drugs, or reagents are suitable for administering in combination with the compounds of the present invention, including without limitation, anti-inflammatory agents, e.g., non-steroidal anti-inflammatory drugs (NSAIDs), corticosteroids, TNF blockers or inhibitors, IL-RA, azathioprine, cyclophosphamide, sulfasalazine; agents and treatments for allegeric diseases, agents for treating asthma, e.g., albuterol, Singulair®; agents for treating multiple sclerosis, e.g., β-interferon (e.g., Avonex®, Rebif®), Copaxone®, mitoxantrone; immunosuppressive and immunomodulatory agents, e.g., cyclosporin, tacrolimus, rapamycin, mycophenolate mofetil, interferons, corticosteroids, cyclophosphamide, azathioprine, sulfasalazine; cardiovascular disease treatment agents, e.g., ACE inhibitors, beta-blockers, diuretics, nitrates, calcium channel blockers, statins; diabetes treatment agents, e.g., insulin, glitazones, sulfonyl ureas; and blood disorder treatment agents, e.g., corticosteroids, and anti-leukemia agents.

The amount of additional therapeutic agent, compound, drug, or reagent present in the compositions of the invention, or administered in conjunction with the compounds of the invention, are no more than the amount which would normally be administered in a composition comprising that therapeutic agent, compound, drug, or reagent as the only active agent. As a guide, the amount of additional therapeutic agent, compound, drug, or reagent in a composition according to the present invention will range from about 40%-100% of the amount normally present in a composition comprising that agent, compound, drug, or reagent as the only therapeutically active agent.

In one embodiment of the invention, should a need or desire arise, a compound according to the invention, or a stereoisomer or prodrug thereof, is combined with an anti-diarrhea agent that is loperamide, loperamide analogs, N-oxides of loperamide and analogs, metabolites and prodrugs thereof, diphenoxylate, cisapride, antacids, aluminum hydroxide, magnesium aluminum silicate, magnesium carbonate, magnesium hydroxide, calcium carbonate, polycarbophil, simethicone, hyoscyamine, atropine, furazolidone, difenoxin, octreotide, lansoprazole, omeprazole and enantiomer, kaolin, pectin, activated charcoal, sulphaguanidine, succinylsulphathiazole, phthalylsulphathiazole, bismuth aluminate, bismuth subcarbonate, bismuth subcitrate, bismuth citrate, tripotassium dicitrato bismuthate, bismuth tartrate, bismuth subsalicylate, bismuth subnitrate and bismuth subgallate, opium tincture (paregoric), herbal medicines, plant-derived anti-diarrheal agents or combinations thereof.

The pharmaceutical preparations of the present invention may include, or be diluted into, a pharmaceutically-acceptable carrier. The term “pharmaceutically-acceptable carrier” as used herein means one or more compatible solid, gel, or liquid fillers, diluents or encapsulating substances which are suitable for administration to a human or other mammal such as a non-human primate, a dog, cat, horse, cow, sheep, pig, or goat. The term “carrier” denotes an organic or inorganic ingredient, natural or synthetic, with which the active ingredient is combined to facilitate the application. The carriers are capable of being commingled with the compositions, compounds and preparations of the present invention, and with each other, in a manner such that there is no interaction which would substantially impair the desired pharmaceutical efficacy or stability. Carrier formulations suitable for oral administration, for suppositories, and for parenteral administration, etc., can be found in Remington's Pharmaceutical Sciences, Mack Publishing Company, Easton, Pa.

Aqueous formulations may include a chelating agent, a buffering agent, an anti-oxidant and, optionally, an isotonicity agent. In an embodiment, the formulation is pH adjusted to between 3.0 and 3.5.

Chelating agents include, for example, but are not limited to ethylenediaminetetraacetic acid (EDTA) as a free acid, salt or various combinations and derivatives thereof, citric acid and derivatives thereof, niacinamide and derivatives thereof, sodium desoxycholate and derivatives thereof, and L-glutamic acid, N,N-diacetic acid and derivatives thereof.

Buffering agents include but are not limited to, citric acid, sodium citrate, sodium acetate, acetic acid, sodium phosphate and phosphoric acid, sodium ascorbate, tartaric acid, maleic acid, glycine, sodium lactate, lactic acid, ascorbic acid, imidazole, sodium bicarbonate and carbonic acid, sodium succinate and succinic acid, histidine, and sodium benzoate and benzoic acid, or combinations thereof.

Antioxidants include, for example, an ascorbic acid derivative, butylated hydroxy anisole, butylated hydroxy toluene, alkyl gallate, sodium meta-bisulfite, sodium bisulfite, sodium dithionite, sodium thioglycollate acid, sodium formaldehyde sulfoxylate, tocopheral and derivatives thereof, monothioglycerol, or sodium sulfite or combinations thereof In one embodiment, the antioxidant is monothioglycerol.

Illustrative isotonicity agents include, but are not limited to, sodium chloride, mannitol, lactose, dextrose, glycerol, or sorbitol, or combinations thereof.

Preservatives that can be used with the present compositions include without limitation benzyl alcohol, parabens, thimerosal, chlorobutanol and preferably benzalkonium chloride. Typically, the preservative will be present in a composition in a concentration of up to about 2% by weight. The exact concentration of the preservative, however, will vary depending upon the intended use and can be easily ascertained by one skilled in the art.

The compounds of the invention can be prepared in lyophilized compositions, typically in the presence of a cryoprotecting agent such as mannitol, or lactose, sucrose, polyethylene glycol, and polyvinyl pyrrolidines. Cryoprotecting agents which result in a reconstitution pH of 6.0 or less are desired. The invention therefore provides a lyophilized preparation of the therapeutic agent(s) of the invention. The preparation can contain a cryoprotecting agent, such as mannitol or lactose, which is preferably neutral or acidic in water.

Oral, parenteral and suppository formulations of agents are well known and commercially available. The therapeutic compound(s) of the invention can be added to such well known formulations. One or more compounds of the invention can be mixed together in solution or semi-solid solution in such formulations, provided in a suspension within such formulations, or contained in particles within such formulations. As used herein, “prodrug” refers to compounds specifically designed to maximize the amount of active species that reaches the desired site of reaction that are of themselves typically inactive or minimally active for the activity desired, but through biotransformation are converted into biologically active metabolites.

As used herein, “pharmaceutically acceptable” refers to those compounds, materials, compositions, and/or dosage forms that are, within the scope of sound medical judgment, suitable for contact with the tissues of human beings and animals without a resulting or excessive toxicity, irritation, allergic response, or other problem complications commensurate with a reasonable benefit/risk ratio. As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues, such as amines, alkali or organic salts of acidic residues, such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine. Certain acidic or basic compounds of the present invention may exist as zwitterions. All forms of the compounds, including free acid, free base and zwitterions, are contemplated to be within the scope of the present invention. It is well known in the art that compounds containing both amino and carboxyl groups often exist in equilibrium with their zwitterionic forms. Thus, any of the compounds described herein that contain, for example, both amino and carboxyl groups, also include reference to their corresponding zwitterions.

A product containing therapeutic compound(s) of the invention and, optionally, one or more other active agents can be configured as an oral dosage. The oral dosage may be a liquid, a semisolid or a solid. The oral dosage may be configured to release the therapeutic compound(s) of the invention before, after or simultaneously with the other agent. The oral dosage may be configured to have the therapeutic compound(s) of the invention and the other agents release completely in the stomach, release partially in the stomach and partially in the intestine, in the intestine, in the colon, partially in the stomach, or wholly in the colon. The oral dosage also may be configured whereby the release of the therapeutic compound(s) of the invention is confined to the stomach or intestine while the release of the other active agent is not so confined or is confined differently from the therapeutic compound(s) of the invention. For example, the therapeutic compound(s) of the invention may be an enterically coated core or pellets contained within a pill or capsule that releases the other agent first and releases the therapeutic compound(s) of the invention only after the therapeutic compound(s) of the invention passes through the stomach and into the intestine. The therapeutic compound(s) of the invention also can be in a sustained release material, whereby the therapeutic compound(s) of the invention is released throughout the gastrointestinal tract and the other agent is released on the same or a different schedule. The same objective for therapeutic compound(s) of the invention release can be achieved with immediate release of therapeutic compound(s) of the invention combined with enteric coated therapeutic compound(s) of the invention. In these instances, the other compound or agent could be released immediately in the stomach, throughout the gastrointestinal tract or only in the intestine.

The materials useful for achieving these different release profiles are well known to those of ordinary skill in the art. Immediate release is obtainable by conventional tablets with binders which dissolve in the stomach. Coatings which dissolve at the pH of the stomach, or which dissolve at elevated temperatures, will achieve the same purpose. Release only in the intestine is achieved using conventional enteric coatings such as pH sensitive coatings which dissolve in the pH environment of the intestine (but not the stomach), or coatings that dissolve over time. Release throughout the gastrointestinal tract is achieved by using sustained-release materials and/or combinations of the immediate release systems and sustained and/or delayed intentional release systems (e.g., pellets which dissolve at different pHs).

In the event that it is desirable to release the therapeutic compound(s) of the invention first, the therapeutic compound(s) of the invention could be coated on the surface of the controlled release formulation in any pharmaceutically acceptable carrier suitable for such coatings and for permitting the release of the therapeutic compound(s) of the invention, such as in a temperature sensitive pharmaceutically acceptable carrier routinely used for controlled release. Other coatings, which dissolve when placed in the body, are well known to those of ordinary skill in the art.

The therapeutic compound(s) of the invention also may be mixed throughout a controlled release formulation, whereby it is released before, after, or simultaneously with another agent. The therapeutic compound(s) of the invention may be free, that is, solubilized within the material of the formulation. The therapeutic compound(s) of the invention also may be in the form of vesicles, such as wax-coated micropellets dispersed throughout the material of the formulation. The coated pellets can be fashioned to immediately release the therapeutic compound(s) of the invention based on temperature, pH,.or the like. The pellets also can be configured so as to delay the release of the therapeutic compound(s) of the invention, allowing the other agent a period of time to act before the therapeutic compound(s) of the invention exerts its effects. The therapeutic compound(s) of the invention also can be configured, e.g., as pellets, to release the therapeutic compound(s) of the invention in virtually any sustained release pattern, including patterns exhibiting first order release kinetics or sigmoidal order release kinetics using materials of the prior art and well known to those of ordinary skill in the art.

The therapeutic compound(s) of the invention also can be contained within a core within the controlled release formulation. The core may have any one or any combination of the properties described above in connection with the pellets. The therapeutic compound(s) of the invention may be, for example, in a core coated with a material, dispersed throughout a material, coated onto a material or adsorbed into or throughout a material.

It should be understood that the pellets or core may be of virtually any type. They may be drug coated with a release material, drug interspersed throughout material, drug adsorbed into a material, and so on. The material may be erodible or nonerodible.

The therapeutic compound(s) of the invention may be provided in particles. Particles as used herein means nano or microparticles (or in some instances larger) which can consist in whole or in part of the therapeutic compound(s) of the invention or the other agents as described herein. The particles may contain the therapeutic compound(s) / agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the antagonist in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in a controlled release formulation or controlled release systems. The term “controlled release” is intended to refer to any drug-containing formulation in which the manner and profile of drug release from the formulation are controlled. This refers to immediate as well as nonimmediate release formulations, with nonimmediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.” These formulations may be for any mode of administration.

Delivery systems specific for the gastrointestinal tract are roughly divided into three types: the first is a delayed release system designed to release a drug in response to, for example, a change in pH; the second is a timed-release system designed to release a drug after a predetermined time; and the third is a microflora enzyme system making use of the abundant enterobacteria in the lower part of the gastrointestinal tract (e.g., in a colonic site-directed release formulation).

An example of a delayed release system is one that uses, for example, an acrylic or cellulosic coating material and dissolves on pH change. Because of ease of preparation, many reports on such “enteric coatings” have been made. In general, an enteric coating is one which passes through the stomach without releasing substantial amounts of drug in the stomach (i.e., less than 10% release, 5% release and even 1% release in the stomach) and sufficiently disintegrating in the intestinal tract (by contact with approximately neutral or alkaline intestine juices) to allow the transport (active or passive) of the active agent through the walls of the intestinal tract.

Various in vitro tests for determining whether or not a coating is classified as an enteric coating have been published in the pharmacopoeia of various countries. A coating which remains intact for at least 2 hours, in contact with artificial gastric juices such as HCl of pH 1 at 36 to 38° C. and thereafter disintegrates within 30 minutes in artificial intestinal juices such as a KH₂PO₄ buffered solution of pH 6.8 is one example. One such well known system is EUDRAGIT material, commercially available and reported on by Boehringer, Manchester University, Saale Co., and the like. Enteric coatings are discussed further below.

A timed release system is represented by Time Erosion System (TES) by Fujisawa Pharmaceutical Co., Ltd. and Pulsincap by R. P. Scherer. According to these systems, the site of drug release is decided by the time of transit of a preparation in the gastrointestinal tract. Since the transit of a preparation in the gastrointestinal tract is largely influenced by the gastric emptying time, some time release systems are also enterically coated.

Systems making use of the enterobacteria can be classified into those utilizing degradation of azoaromatic polymers by an azo reductase produced from enterobacteria as reported by a group at Ohio University (M. Saffran, et al., Science, Vol. 233: 1081 (1986)) and a group at Utah University (J. Kopecek, et al., Pharmaceutical Research, 9(12), 1540-1545 (1992)); and those utilizing degradation of polysaccharides by beta-galactosidase of enterobacteria as reported by a group a Hebrew University (unexamined published Japanese patent application No. 5-50863 based on a PCT application) and a group at Freiberg University (K. H. Bauer et al., Pharmaceutical Research, 10(10), S218 (1993)). In addition, the system using chitosan degradable by chitosanase by Teikoku Seiyaku K. K. (unexamined published Japanese patent application No. 4-217924 and unexamined published Japanese patent application No. 4-225922) is also included.

The enteric coating is typically, although not necessarily, a polymeric material. Preferred enteric coating materials comprise bioerodible, gradually hydrolyzable and/or gradually water-soluble polymers. The “coating weight,” or relative amount of coating material per capsule, generally dictates the time interval between ingestion and drug release. Any coating should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention. The selection of the specific enteric coating material will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; ease of application as a coating (substrate friendly); and economical practicality.

Suitable enteric coating materials include, but are not limited to: cellulosic polymers such as cellulose acetate phthalate, cellulose acetate trimellitate, hydroxypropylmethyl cellulose phthalate, hydroxypropyhmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl methacrylate (e.g., those copolymers sold under the trade name EUDRAGIT); vinyl polymers and copolymers such as polyvinyl acetate, polyvinylacetate phthalate, vinylacetate crotonic acid copolymer, and ethylene-vinyl acetate copolymers; and shellac (purified lac). Combinations of different coating materials may also be used. Well known enteric coating material for use herein are those acrylic acid polymers and copolymers available under the trade name EUDRAGIT from Rohm Pharma (Germany). The EUDRAGIT series E, L, S, RL, RS and NE copolymers are available as solubilized in organic solvent, as an aqueous dispersion, or as a dry powder. The EUDRAGIT series RL, NE, and RS copolymers are insoluble in the gastrointestinal tract but are permeable and are used primarily for extended release. The EUDRAGIT series E copolymers dissolve in the stomach. The EUDRAGIT series L, L-30D and (S) copolymers are insoluble in stomach and dissolve in the intestine, and are thus most preferred herein.

A particular methacrylic copolymer is EUDRAGIT L, particularly L-30D and EUDRAGIT L 100-55. In EUDRAGIT L-30D, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH generally present in the fluid of lower gastrointestinal tract. Another particular methacrylic acid polymer is EUDRAGIT S, which differs from EUDRAGIT L-30D in that the ratio of free carboxyl groups to ester groups is approximately 1:2. EUDRAGIT (S) is insoluble at pH below 5.5, but unlike EUDRAGIT L-30D, is poorly soluble in gastrointestinal fluids having a pH in the range of 5.5 to 7.0, such as in the small intestine. This copolymer is soluble at pH 7.0 and above, i.e., the pH generally found in the colon. EUDRAGIT (S) can be used alone as a coating to provide drug delivery in the large intestine. Alternatively, EUDRAGIT S, being poorly soluble in intestinal fluids below pH 7, can be used in combination with EUDRAGIT L-30D, soluble in intestinal fluids above pH 5.5, in order to provide a delayed release composition which can be formulated to deliver the active agent to various segments of the intestinal tract. The more EUDRAGIT L-30D used, the more proximal release and delivery begins, and the more EUDRAGIT (S) used, the more distal release and delivery begins. It will be appreciated by those skilled in the art that both EUDRAGIT L-30D and EUDRAGIT (S) can be replaced with other pharmaceutically acceptable polymers having similar pH solubility characteristics. In certain embodiments of the invention, the preferred enteric coating is ACRYL-EZE™ (methacrylic acid co-polymer type C; Colorcon, West Point, Pa.).

The enteric coating provides for controlled release of the active agent, such that drug release can be accomplished at some generally predictable location. The enteric coating also prevents exposure of the therapeutic agent and carrier to the epithelial and mucosal tissue of the buccal cavity, pharynx, esophagus, and stomach, and to the enzymes associated with these tissues. The enteric coating therefore helps to protect the active agent, carrier and a patient's internal tissue from any adverse event prior to drug release at the desired site of delivery. Furthermore, the coated material of the present invention allows optimization of drug absorption, active agent protection, and safety. Multiple enteric coatings targeted to release the active agent at various regions in the gastrointestinal tract would enable even more effective and sustained improved delivery throughout the gastrointestinal tract.

The coating can, and usually does, contain a plasticizer to prevent the formation of pores and cracks that would permit the penetration of the gastric fluids. Suitable plasticizers include, but are not limited to, triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating comprised of an anionic carboxylic acrylic polymer will usually contain approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The coating can be applied to particles of the therapeutic agent(s), tablets of the therapeutic agent(s), capsules containing the therapeutic agent(s) and the like, using conventional coating methods and equipment. For example, an enteric coating can be applied to a capsule using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6th Ed. (Media, P A: Williams & Wilkins, 1995). The coating thickness, as noted above, must be sufficient to ensure that the oral dosage form remains intact until the desired site of topical delivery in the lower intestinal tract is reached.

In another embodiment, drug dosage forms are provided that comprise an enterically coated, osmotically activated device housing a formulation of the invention. In this embodiment, the drug-containing formulation is encapsulated in a semipermeable membrane or barrier containing a small orifice. As known in the art with respect to so-called “osmotic pump” drug delivery devices, the semipermeable membrane allows passage of water in either direction, but not drug. Therefore, when the device is exposed to aqueous fluids, water will flow into the device due to the osmotic pressure differential between the interior and exterior of the device. As water flows into the device, the drug-containing formulation in the interior will be “pumped” out through the orifice. The rate of drug release will be equivalent to the inflow rate of water times the drug concentration. The rate of water influx and drug efflux can be controlled by the composition and size of the orifice of the device. Suitable materials for the semipermeable membrane include, but are not limited to, polyvinyl alcohol, polyvinyl chloride, semipermeable polyethylene glycols, semipermeable polyurethanes, semipermeable polyamides, semipermeable sulfonated polystyrenes and polystyrene derivatives; semipermeable poly(sodium styrenesulfonate), semipermeable poly(vinylbenzyltrimethylammonium chloride), and cellulosic polymers such as cellulose acetate, cellulose diacetate, cellulose triacetate, cellulose propionate, cellulose acetate propionate, cellulose acetate butyrate, cellulose trivalerate, cellulose trilmate, cellulose tripalmitate, cellulose trioctanoate, cellulose tripropionate, cellulose disuccinate, cellulose dipalmitate, cellulose dicylate, cellulose acetate succinate, cellulose propionate succinate, cellulose acetate octanoate, cellulose valerate palmitate, cellulose acetate heptanate, cellulose acetaldehyde dimethyl acetal, cellulose acetate ethylcarbamate, cellulose acetate methylcarbamate, cellulose dimethylaminoacetate and ethylcellulose.

In another embodiment, drug dosage forms are provided that comprise a sustained release coated device housing a formulation of the invention. In this embodiment, the drug-containing formulation is encapsulated in a sustained release membrane or film. The membrane may be semipermeable, as described above. A semipermeable membrane allows for the passage of water inside the coated device to dissolve the drug. The dissolved drug solution diffuses out through the semipermeable membrane. The rate of drug release depends upon the thickness of the coated film and the release of drug can begin in any part of the GI tract. Suitable membrane materials for such a membrane include ethylcellulose.

In another embodiment, drug dosage forms are provided that comprise a sustained release device housing a formulation of the invention. In this embodiment, the drug-containing formulation is uniformly mixed with a sustained release polymer. These sustained release polymers are high molecular weight water-soluble polymers, which when in contact with water, swell and create channels for water to diffuse inside and dissolve the drug. As the polymers swell and dissolve in water, more of drug is exposed to water for dissolution. Such a system is generally referred to as sustained release matrix. Suitable materials for such a device include hydropropyl methylcellulose, hydroxypropyl cellulose, hydroxyethyl cellulose and methyl cellulose.

In another embodiment, drug dosage forms are provided that comprise an enteric coated device housing a sustained release formulation of the invention. In this embodiment, the drug containing product described above is coated with an enteric polymer. Such a device would not release any drug in the stomach and when the device reaches the intestine, the enteric polymer is first dissolved and only then would the drug release begin. The drug release would take place in a sustained release fashion.

Enterically coated, osmotically activated devices can be manufactured using conventional materials, methods and equipment. For example, osmotically activated devices may be made by first encapsulating, in a pharmaceutically acceptable soft capsule, a liquid or semi-solid formulation of the compounds of the invention as described previously. This interior capsule is then coated with a semipermeable membrane composition (comprising, for example, cellulose acetate and polyethylene glycol 4000 in a suitable solvent such as a methylene chloride-methanol admixture), for example using an air suspension machine, until a sufficiently thick laminate is formed, e.g., around 0.05 mm. The semipermeable laminated capsule is then dried using conventional techniques. Then, an orifice having a desired diameter (e.g., about 0.99 mm) is provided through the semipermeable laminated capsule wall, using, for example, mechanical drilling, laser drilling, mechanical rupturing, or erosion of an erodible element such as a gelatin plug. The osmotically activated device may then be enterically coated as previously described. For osmotically activated devices containing a solid carrier rather than a liquid or semi-solid carrier, the interior capsule is optional; that is, the semipermeable membrane may be formed directly around the carrier-drug composition. However, preferred carriers for use in the drug-containing formulation of the osmotically activated device are solutions, suspensions, liquids, immiscible liquids, emulsions, sols, colloids, and oils. Particularly preferred carriers include, but are not limited to, those used for enterically coated capsules containing liquid or semisolid drug formulations.

Cellulose coatings include those of cellulose acetate phthalate and trimellitate; methacrylic acid copolymers, e.g. copolymers derived from methylacrylic acid and esters thereof, containing at least 40% methylacrylic acid; and especially hydroxypropyl methylcellulose phthalate. Methylacrylates include those of molecular weight above 100,000 daltons based on, e.g. methylacrylate and methyl or ethyl methylacrylate in a ratio of about 1:1. Typical products include Eudragit L, e.g. L 100-55, marketed by Rohm GmbH, Darmstadt, Germany. Typical cellulose acetate phthalates have an acetyl content of 17-26% and a phthalate content of from 30-40% with a viscosity of ca. 45-90 cP. Typical cellulose acetate trimellitates have an acetyl content of 17-26%, a trimellityl content from 25-35% with a viscosity of ca. 15-20 cS. An example of a cellulose acetate trimellitate is the marketed product CAT (Eastman Kodak Company, USA). Hydroxypropyl methylcellulose phthalates typically have a molecular weight of from 20,000 to 130,000 daltons, a hydroxypropyl content of from 5 to 10%, a methoxy content of from 18 to 24% and a phthalyl content from 21 to 35%. An example of a cellulose acetate phthalate is the marketed product CAP (Eastman Kodak, Rochester N.Y., USA). Examples of hydroxypropyl methylcellulose phthalates are the marketed products having a hydroxypropyl content of from 6-10%, a methoxy content of from 20-24%, a phthalyl content of from 21-27%, a molecular weight of about 84,000 daltons, sold under the trademark HP50 and available from Shin-Etsu Chemical Co. Ltd., Tokyo, Japan, and having a hydroxypropyl content, a methoxyl content, and a phthalyl content of 5-9%, 18-22% and 27-35%, respectively, and a molecular weight of 78,000 daltons, known under the trademark HP55 and available from the same supplier.

The therapeutic agents may be provided in coated or uncoated capsules. The capsule material may be either hard or soft, and as will be appreciated by those skilled in the art, typically comprises a tasteless, easily administered and water soluble compound such as gelatin, starch or a cellulosic material. The capsules are preferably sealed, such as with gelatin bands or other biologically amenable sealant material. See, for example, Remington: The Science and Practice of Pharmacy, Nineteenth Edition (Easton, Pa.: Mack Publishing Co., 1995), which describes materials and methods for preparing encapsulated pharmaceuticals.

A product containing therapeutic compound(s) of the invention can be configured as a suppository. The therapeutic compound(s) of the invention can be placed anywhere within or on the suppository to favorably affect the relative release of the therapeutic compound(s). The nature of the release can be zero order, first order, or sigmoidal, as desired.

Suppositories are solid dosage forms of medicine intended for administration via the rectum. Suppositories are compounded so as to melt, soften, or dissolve in the body cavity (around 98.6° F.) thereby releasing the medication contained therein. Suppository bases should be stable, nonirritating, chemically inert, and physiologically inert. Many commercially available suppositories contain oily or fatty base materials, such as cocoa butter, coconut oil, palm kernel oil, and palm oil, which often melt or deform at room temperature necessitating cool storage or other storage limitations. U.S. Pat. No. 4,837,214 to Tanaka et al. describes a suppository base comprised of 80 to 99 percent by weight of a lauric-type fat having a hydroxyl value of 20 or smaller and containing glycerides of fatty acids having 8 to 18 carbon atoms combined with 1 to 20 percent by weight diglycerides of fatty acids (which erucic acid is an example of). The shelf life of these type of suppositories is limited due to degradation. Other suppository bases contain alcohols, surfactants, and such diluents which raise the melting temperature but also can lead to poor absorption of the medicine and side effects due to irritation of the local mucous membranes (see for example, U.S. Pat. No. 6,099,853 to Hartelendy et al., U.S. Pat. No. 4,999,342 to Ahmad et al., and U.S. Pat. No. 4,765,978 to Abidi et al.).

The base used in the pharmaceutical suppository composition of this invention includes, in general, oils and fats comprising triglycerides as main components such as cacao butter, palm fat, palm kernel oil, coconut oil, fractionated coconut oil, lard and WITEPSOL®, waxes such as lanolin and reduced lanolin; hydrocarbons such as VASELINE®, squalene, squalane and liquid paraffin; long to medium chain fatty acids such as caprylic acid, lauric acid, stearic acid and oleic acid; higher alcohols such as lauryl alcohol, cetanol and stearyl alcohol; fatty acid esters such as butyl stearate and dilauryl malonate; medium to long chain carboxylic acid esters of glycerin such as triolein and tristearin; glycerin-substituted carboxylic acid esters such as glycerin acetoacetate; and polyethylene glycols and its derivatives, such as macrogols and cetomacrogol. They may be used either singly or in combination of two or more. If desired, the composition of this invention may further include a surface-active agent, a coloring agent, etc., which are ordinarily used in suppositories.

The pharmaceutical compositions of this invention may be prepared by uniformly mixing predetermined amounts of the active ingredient, the absorption aid and optionally the base, etc. in a stirrer or a grinding mill, at an elevated temperature if required. The resulting composition may be formed into a suppository in unit dosage form by, for example, casting the mixture in a mold, or by forming it into a gelatin capsule using a capsule filling machine.

The compositions according to the present invention also can be administered as a nasal spray, nasal drop, suspension, gel, ointment, cream or powder. The administration of a composition can also include using a nasal tampon or a nasal sponge containing or impregnated with a composition of the present invention.

The nasal delivery systems that can be used with the present invention can take various forms including aqueous preparations, non-aqueous preparations and combinations thereof. Aqueous preparations include, for example, aqueous gels, aqueous suspensions, aqueous liposomal dispersions, aqueous emulsions, aqueous microemulsions and combinations thereof. Non-aqueous preparations include, for example, non-aqueous gels, non-aqueous suspensions, non-aqueous liposomal dispersions, non-aqueous emulsions, non-aqueous microemulsions and combinations thereof. The various forms of the nasal delivery systems can include a buffer to maintain pH, a pharmaceutically acceptable thickening agent and a humectant. The pH of the buffer can be selected to optimize the absorption of the therapeutic agent(s) across the nasal mucosa.

With respect to the non-aqueous nasal formulations, suitable forms of buffering agents can be selected such that when the formulation is delivered into the nasal cavity of a mammal, selected pH ranges are achieved therein upon contact with, e.g., a nasal mucosa. In the present invention, the pH of the compositions should be maintained from about 2.0 to about 6.0. It is desirable that the pH of the compositions is one which does not cause significant irritation to the nasal mucosa of a recipient upon administration. An aerosol or spray device may be used in conjunction with the nasal delivery systems of the invention.

The viscosity of the compositions of the present invention can be maintained at a desired level using a pharmaceutically acceptable thickening agent. Thickening agents that can be used in accordance with the present invention include methyl cellulose, xanthan gum, carboxymethyl cellulose, hydroxypropyl cellulose, carbomer, polyvinyl alcohol, alginates, acacia, chitosans and combinations thereof. The concentration of the thickening agent will depend upon the agent selected and the viscosity desired. Such agents can also be used in a powder formulation discussed above.

The compositions of the present invention can also include a humectant to reduce or prevent drying of the mucus membrane and to prevent irritation thereof Illustratively, suitable humectants that can be used in the present invention include sorbitol, mineral oil, vegetable oil and glycerol; soothing agents; membrane conditioners; sweeteners; and combinations thereof. The concentration of the humectant in the present compositions will vary depending upon the agent selected.

One or more therapeutic agents may be incorporated into the nasal delivery system or any other delivery system described herein.

A composition formulated for topical administration may be liquid or semi-solid (including, for example, a gel, lotion, emulsion, cream, ointment, spray or aerosol) or may be provided in combination with a “finite” carrier, for example, a non-spreading material that retains its form, including, for example, a patch, bioadhesive, dressing or bandage. It may be aqueous or non-aqueous; it may be formulated as a solution, emulsion, dispersion, a suspension or any other mixture.

Some modes of administration include topical application to the skin, eyes or mucosa. Thus, typical vehicles are those suitable for pharmaceutical or cosmetic application to body surfaces. The compositions provided herein may be applied topically or locally to various areas in the body of a patient. As noted above, topical application is intended to refer to application to the tissue of an accessible body surface, such as, for example, the skin (the outer integument or covering) and the mucosa (the mucous-producing, secreting and/or containing surfaces). Exemplary mucosal surfaces include the mucosal surfaces of the eyes, mouth (such as the lips, tongue, gums, cheeks, sublingual and roof of the mouth), larynx, esophagus, bronchial, nasal passages, vagina and rectum/anus; in some embodiments, preferably the mouth, larynx, esophagus, vagina and rectum/anus; in other embodiments, preferably the eyes, larynx, esophagus, bronchial, nasal passages, and vagina and rectum/anus. As noted above, local application herein refers to application to a discrete internal area of the body, such as, for example, a joint, soft tissue area (such as muscle, tendon, ligaments, intraocular or other fleshy internal areas), or other internal area of the body. Thus, as used herein, local application refers to applications to discrete areas of the body.

Also in certain embodiments, including embodiments that involve aqueous vehicles, the compositions may also contain a glycol, that is, a compound containing two or more hydroxy groups. A glycol which is particularly preferred for use in the compositions is propylene glycol. In these embodiments, the glycol is preferably included in the compositions in a concentration of from greater than 0 to about 5 wt. %, based on the total weight of the composition. More preferably, the compositions contain from about 0.1 to less than about 5 wt. % of a glycol, with from about 0.5 to about 2 wt. % being even more preferred. Still more preferably, the compositions contain about 1 wt. % of a glycol.

For local internal administration, such as intra-articular administration, the compositions are preferably formulated as a solution or a suspension in an aqueous-based medium, such as isotonically buffered saline or are combined with a biocompatible support or bioadhesive intended for internal administration.

Lotions, which, for example, may be in the form of a suspension, dispersion or emulsion, contain an effective concentration of one or more of the compounds. The effective concentration is preferably to deliver an effective amount, typically at a concentration of between about 0.1-50%, by weight, or more of one or more of the compounds provided herein. The lotions also contain by weight from 1% to 50% of an emollient and the balance water, a suitable buffer, and other agents as described above. Any emollients known to those of skill in the art as suitable for application to human skin may be used. These include, but are not limited to, the following: (a) Hydrocarbon oils and waxes, including mineral oil, petrolatum, paraffin, ceresin, ozokerite, microcrystalline wax, polyethylene, and perhydrosqualene. b) Silicone oils, including dimethylpolysiloxanes, methylphenylpolysiloxanes, water-soluble and alcohol-soluble silicone-glycol copolymers. (c) Triglyceride fats and oils, including those derived from vegetable, animal and marine sources. Examples include, but are not limited to, castor oil, safflower oil, cotton seed oil, corn oil, olive oil, cod liver oil, almond oil, avocado oil, palm oil, sesame oil, and soybean oil. (d) Acetoglyceride esters, such as acetylated monoglycerides. (e) Ethoxylated glycerides, such as ethoxylated glyceryl monostearate. (f) Alkyl esters of fatty acids having 10 to 20 carbon atoms. Methyl, isopropyl and butyl esters of fatty acids are useful herein. Examples include, but are not limited to, hexyl laurate, isohexyl laurate, isohexyl palmitate, isopropyl palmitate, isopropyl myristate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate. (g) Alkenyl esters of fatty acids having 10 to 20 carbon atoms. Examples thereof include, but are not limited to, oleyl myristate, oleyl stearate, and oleyl oleate. (h) Fatty acids having 9 to 22 carbon atoms. Suitable examples include, but are not limited to, pelargonic, lauric, myristic, palmitic, stearic, isostearic, hydroxystearic, oleic, linoleic, ricinoleic, arachidonic, behenic, and erucic acids. (i) Fatty alcohols having 10 to 22 carbon atoms, such as, but not limited to, lauryl, myristyl, cetyl, hexadecyl, stearyl, isostearyl, hydroxystearyl, oleyl, ricinoleyl, behenyl, erucyl, and 2-octyl dodecyl alcohols. (j) Fatty alcohol ethers, including, but not limited to ethoxylated fatty alcohols of 10 to 20 carbon atoms, such as, but are not limited to, the lauryl, cetyl, stearyl, isostearyl, oleyl, and cholesterol alcohols having attached thereto from 1 to 50 ethylene oxide groups or 1 to 50 propylene oxide groups or mixtures thereof. (k) Ether-esters, such as fatty acid esters of ethoxylated fatty alcohols. (l) Lanolin and derivatives, including, but not limited to, lanolin, lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols, lanolin alcohols linoleate, lanolin alcohols ricinoleate, acetate of lanolin alcohols ricinoleate, acetate of ethoxylated alcohol(S)-esters, hydrogenolysis of lanolin, ethoxylated hydrogenated lanolin, ethoxylated sorbitol lanolin, and liquid and semisolid lanolin absorption bases. (m) polyhydric alcohols and polyether derivatives, including, but not limited to, propylene glycol, dipropylene glycol, polypropylene glycol [M.W. 2000-4000], polyoxyethylene polyoxypropylene glycols, polyoxypropylene polyoxyethylene glycols, glycerol, ethoxylated glycerol, propoxylated glycerol, sorbitol, ethoxylated sorbitol, hydroxypropyl sorbitol, polyethylene glycol [M.W. 200-6000], methoxy polyethylene glycols 350, 550, 750, 2000, 5000, poly(ethylene oxide) homopolymers [M.W. 100,000-5,000,000], polyalkylene glycols and derivatives, hexylene glycol (2-methyl-2,4-pentanediol), 1,3-butylene glycol, 1,2,6,-hexanetriol, ethohexadiol USP (2-ethyl-1,3-hexanediol), C.sub.15 -C.sub.18 vicinal glycol and polyoxypropylene derivatives of trimethylolpropane. (n) polyhydric alcohol esters, including, but not limited to, ethylene glycol mono- and di-fatty acid esters, diethylene glycol mono- and di-fatty acid esters, polyethylene glycol [M.W. 200-6000], mono- and di-fatty esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters. (o) Wax esters, including, but not limited to, beeswax, spermaceti, myristyl myristate, and stearyl stearate and beeswax derivatives, including, but not limited to, polyoxyethylene sorbitol beeswax, which are reaction products of beeswax with ethoxylated sorbitol of varying ethylene oxide content that form a mixture of ether-esters. (p) Vegetable waxes, including, but not limited to, carnauba and candelilla waxes. (q) phospholipids, such as lecithin and derivatives. (r) Sterols, including, but not limited to, cholesterol and cholesterol fatty acid esters. (s) Amides, such as fatty acid amides, ethoxylated fatty acid amides, and solid fatty acid alkanolamides.

The lotions further preferably contain, by weight, from 1% to 10%, or preferably from 2% to 5%, of an emulsifier. The emulsifiers can be nonionic, anionic or cationic. Examples of satisfactory nonionic emulsifiers include, but are not limited to, fatty alcohols having 10 to 20 carbon atoms, fatty alcohols having 10 to 20 carbon atoms condensed with 2 to 20 moles of ethylene oxide or propylene oxide, alkyl phenols with 6 to 12 carbon atoms in the alkyl chain condensed with 2 to 20 moles of ethylene oxide, mono- and di-fatty acid esters of ethylene oxide, mono- and di-fatty acid esters of ethylene glycol where the fatty acid moiety contains from 10 to 20 carbon atoms, diethylene glycol, polyethylene glycols of molecular weight 200 to 6000, propylene glycols of molecular weight 200 to 3000, glycerol, sorbitol, sorbitan, polyoxyethylene sorbitol, polyoxyethylene sorbitan and hydrophilic wax esters. Suitable anionic emulsifiers include, but are not limited to, the fatty acid soaps, e.g., sodium, potassium and triethanolamine soaps, where the fatty acid moiety contains from 10 to 20 carbon atoms. Other suitable anionic emulsifiers include, but are not limited to, the alkali metal, ammonium or substituted ammonium alkyl sulfates, alkyl arylsulfonates, and alkyl ethoxy ether sulfonates having 10 to 30 carbon atoms in the alkyl moiety. The alkyl ethoxy ether sulfonates contain from 1 to 50 ethylene oxide units. Among satisfactory cationic emulsifiers are quaternary ammonium, morpholinium and pyridinium compounds. Certain of the emollients described in preceding paragraphs also have emulsifying properties. When a lotion is formulated containing such an emollient, an additional emulsifier is not needed, though it can be included in the composition.

The balance of the lotion is water or a C₂ or C₃ alcohol, or a mixture of water and the alcohol. The lotions are formulated by simply admixing all of the components together. Preferably the compound, such as loperamide, is dissolved, suspended or otherwise uniformly dispersed in the mixture.

Other conventional components of such lotions may be included. One such additive is a thickening agent at a level from 1% to 10% by weight of the composition. Examples of suitable thickening agents include, but are not limited to: cross-linked carboxypolymethylene polymers, ethyl cellulose, polyethylene glycols, gum tragacanth, gum kharaya, xanthan gums and bentonite, hydroxyethyl cellulose, and hydroxypropyl cellulose.

Creams can be formulated to contain a concentration effective to deliver an effective amount of therapeutic agent(s) of the invention to the treated tissue, typically at between about 0.1%, preferably at greater than 1% up to and greater than 50%, preferably between about 3% and 50%, more preferably between about 5% and 15% therapeutic agent(s) of the invention. The creams also contain from 5% to 50%, preferably from 10% to 25%, of an emollient and the remainder is water or other suitable non-toxic carrier, such as an isotonic buffer. The emollients, as described above for the lotions, can also be used in the cream compositions. The cream may also contain a suitable emulsifier, as described above. The emulsifier is included in the composition at a level from 3% to 50%, preferably from 5% to 20%.

These compositions that are formulated as solutions or suspensions may be applied to the skin, or, may be formulated as an aerosol or foam and applied to the skin as a spray-on. The aerosol compositions typically contain, by weight, from 25% to 80%, preferably from 30% to 50%, of a suitable propellant. Examples of such propellants are the chlorinated, fluorinated and chlorofluorinated lower molecular weight hydrocarbons. Nitrous oxide, carbon dioxide, butane, and propane are also used as propellant gases. These propellants are used as understood in the art in a quantity and under a pressure suitable to expel the contents of the container.

Suitably prepared solutions and suspensions may also be topically applied to the eyes and mucosa. Solutions, particularly those intended for ophthalmic use, may be formulated as 0.01%-10% isotonic solutions, pH about 5-7, with appropriate salts, and preferably containing one or more of the compounds herein at a concentration of about 0.1%, preferably greater than 1%, up to 50% or more. Suitable ophthalmic solutions are known [see, e.g., U.S. Pat. No. 5,116,868, which describes typical compositions of ophthalmic irrigation solutions and solutions for topical application]. Such solutions, which have a pH adjusted to about 7.4, contain, for example, 90-100 mM sodium chloride, 4-6 mM dibasic potassium phosphate, 4-6 mM dibasic sodium phosphate, 8-12 mM sodium citrate, 0.5-1.5 mM magnesium chloride, 1.5-2.5 mM calcium chloride, 15-25 mM sodium acetate, 10-20 mM D.L.-sodium, β-hydroxybutyrate and 5-5.5 mM glucose.

Gel compositions can be formulated by simply admixing a suitable thickening agent to the previously described solution or suspension compositions. Examples of suitable thickening agents have been previously described with respect to the lotions.

The gelled compositions contain an effective amount of therapeutic agent(s) of the invention, typically at a concentration of between about 0.1-50% by weight or more of one or more of the compounds provided herein.; from 5% to 75%, preferably from 10% to 50%, of an organic solvent as previously described; from 0.5% to 20%, preferably from 1% to 10% of the thickening agent; the balance being water or other aqueous or non-aqueous carrier, such as, for example, an organic liquid, or a mixture of carriers.

The formulations can be constructed and designed to create steady state plasma levels. Steady state plasma concentrations can be measured using HPLC techniques, as are known to those of skill in the art. Steady state is achieved when the rate of drug availability is equal to the rate of drug elimination from the circulation. In typical therapeutic settings, the therapeutic agent(s) of the invention will be administered to patients either on a periodic dosing regimen or with a constant infusion regimen. The concentration of drug in the plasma will tend to rise immediately after the onset of administration and will tend to fall over time as the drug is eliminated from the circulation by means of distribution into cells and tissues, by metabolism, or by excretion. Steady state will be obtained when the mean drug concentration remains constant over time. In the case of intermittent dosing, the pattern of the drug concentration cycle is repeated identically in each interval between doses with the mean concentration remaining constant. In the case of constant infusion, the mean drug concentration will remain constant with very little oscillation. The achievement of steady state is determined by means of measuring the concentration of drug in plasma over at least one cycle of dosing such that one can verify that the cycle is being repeated identically from dose to dose. Typically, in an intermittent dosing regimen, maintenance of steady state can be verified by determining drug concentrations at the consecutive troughs of a cycle, just prior to administration of another dose. In a constant infusion regimen where oscillation in the concentration is low, steady state can be verified by any two consecutive measurements of drug concentration.

A therapeutic feature of the compounds of this invention is inhibition of the PI3K family of lipid kinases, particularly PI3Kα, inhibition of the PI3K-related protein kinase family (PIKK) comprising mTOR, hSMG-1, ATR, ATM, DNA-ATR and the potential signal disruption of other growth factor receptors or signaling components that share binding domains with the PI3K or cooperate with PI3K in disease progression. Accordingly, the invention herein is suited for chronic, acute, symptomatic, therapeutic, or prophylactic treatment of human or animal diseases comprising cancers and associated maladies of malignant or benign growth; disorders of metabolism; exaggerated inflammation and allergic responses; cardiovascular diseases; and complications associated with transplantation. Additionally, in an embodiment, the compounds of this invention act as potent and selective dual inhibitors of PI3K and a protein kinase. . In an embodiment, the compound is an inhibitor of PI3Kα, or a dual inhibitor of PI3Kα and a protein kinase. In a further embodiment, the compounds of this invention act as potent and selective inhibitors of a protein kinase.

A compound of the invention, e.g., Formulas (I-IV) as described herein, is therapeutic for oncologic disorders comprising deregulated cell growth, proliferation, cell survival, cell cycle progression, angiogenesis and metastasis that can result in malignant or benign tumor growth and dissemination. In an embodiment, the invention encompasses a method of treating or lessening the severity of cancer and tumors that may be associated or manifested therewith. This includes deregulated growth of all four cell types, namely, epithelial, connective, nervous and muscle cells, which comprise, but are not limited to, the following cancers: adrenal, bladder, genitourinary tract, brain, medulloblastoma, glioblastoma, breast, cervical (endometrial, uterine), colon, colorectal, esophageal, tongue, mouth, pharynx (oral), lip, buccal cavity, head/neck, kidney, liver, lung, NSCLC, ovarian, pancreatic, prostate, rectal, sarcoma, skin (melanoma and Kaposi's sarcoma), melanoma, myeloma, stomach, thyroid, central nervous system and vaginal cancer. Also included are leukemias, such as acute myeloid leukemia, acute lymphocytic leukemia, chronic myeloid leukemia, and chronic lymphocytic leukemia; acute lymphocytic leukemia (ALL), chronic myelogenous leukemia (CML), multiple myeloma, neuroblastoma, glioma, glioblastoma, lymphoma, sarcoma, and tumors commonly referred to and treated as solid tumors. A solid tumor is as understood by one having skill in the art, and is typically characterized as an abnormal mass of tissue that usually does not contain cysts or liquid areas and may be benign or malignant. Different types of solid tumors are named for the types of cells that form them. Examples of solid tumors comprise sarcomas, carcinomas, and lymphomas. Additionally, solid tumors are also designated by the affected areas or organ, such head and neck, breast, ovary, colon, prostate, brain, kidney, liver, adrenal, gastrointestinal, colon carcinoma, colorectal adenoma. In an embodiment, the compounds and compositions of the invention are provided for use in treating or reducing the severity of organ transplantation rejection. In an embodiment, the compounds and compositions of the invention are provided for use in treating stem cell diseases, disorders and conditions associated with deregulated PI3K and/or protein kinase activity, e.g., MELK or MNK or MNK1, activity.

A compound of the invention, i.e., a compound of Formulas (I-IV) described herein, is also therapeutic for benign or malignant cancers, such as wherein genetic aberrations or environmental conditions lead to activation of the PI3K family, such as overexpression or activating or deregulating mutations, e.g., in the gene product of PIK3CA; and/or genetic or environmental alterations that inactivate negative regulators of the PI3K pathway such as the PTEN phosphatase, which hyrodrolyzes the products of PI3K; and/or analogous conditions that activate other growth factor pathways that interact or cooperate with the PI3K pathway and together acerbate the pathology. Cancers and related syndromes identified to possess these genetically- or environmentally-derived aberrations are suitable for treatment with the compounds of the invention described herein and include, but are not limited to, cancers of the thyroid, leukemia, melanoma, prostate, ovary, cervix, lung, colon, rectum, brain, breast, liver, stomach, endometrium; Cowden's syndrome, Bannayan-Reiley-Ruvalcab syndrome, Proteus syndrome, Proteus-like syndrome and Peutz-Jeghers syndrome.

A compound of the invention may also be therapeutic for diseases of inflammation and allergy because PI3Kδ and PI3Kγ are signaling components in cells required to mount an inflammatory response, such as neutrophils, macrophage, mast cells, T-cells, B-cells, plasma cells, dendritic cells and eosinophils. The associated inflammatory diseases or conditions treatable by the invention include, but are not limited to, autoimmune diseases and common arthritis types, including rheumatoid arthritis, osteoarthritis, ankyolsing spondylitis, psoriatic arthritis; psoriasis, systemic lupus erythematosus, glomerulonephritis, scleroderma, general renal failure, inflammatory bowel disease, ulcerative colitis, Crohn's disease, pancreatitis, multiple sclerosis; inflammation due to hyer-responsiveness to cytokine production, chronic obstructive pulmonary, airway or lung disease (COPD, COAD or COLD), acute respiratory distress syndrome (ARDS) and occupation-related diseases such as aluminosis, anthracosis, asbestosis, chalicosis, ptilosis, siderosis, silicosis, tabacosis and byssinosis. Additionally, a compound of the present invention encompasses treatment of parasite-related diseases involving hypereosinophilia. Since PI3Kδ and PI3Kγ are also signaling components in basophils, eosinophils and mast cells, a compound of the invention is also therapeutic for diseases and conditions related to immediate-type hypersensitivity, also referred to as allergic responses, conditions and diseases. These diseases and conditions include, but are not limited to, asthma (extrinsic or intrinsic), asthma related sequelae including small and large airway hyperactivity, bronanaphylaxis, aspirin-induced asthma, allergic airway inflammation, urticaria, Steven-Johnson syndrome, atopic dermatitis, bolus pemphigoid and the like. A compound of the invention is therapeutic for diseases involving neutrophils, macrophages, mast cells, T-cells, B-cells, plasma-cells, basophiles, eosinophiles and mast cells.

A compound of the present invention may also be used in treatment or therapy for metabolic diseases, such as diabetes and obesity, especially since the PI3K/PKB/Akt pathway signals through the mammalian Target of Rapamycin (mTOR) and thereby contributes to the regulation of fat metabolism. Aberrant fat metabolism contributes directly to obesity and indirectly to type-2 diabetes and the proper homeostatic management of glucose and insulin.

Cardiovascular diseases, acute heart failure, enlargement of the heart, and atherosclerosis are also diseases that are suitable for treatment or therapy using a compound of the invention described herein, since, without wishing to be bound by theory, disruption of PI3Kγ and PI3Kδ reduces infarct size and reprofusion injury. Additionally, a compound of the invention may be therapeutic for atherosclerosis, since, without wishing to be bound by theory, disruption of PI3Kγ inhibits downstream signaling of oxidized LDL, a necessary component in the progression of the disease. Nonlimiting examples of cardiovascular diseases also include pulmonary hypertension, deep venous thrombosis, stroke, myocardial infarction, myocardial contractility diseases or disorders, ischemia, thromboembolism, pulmonary embolism, acute arterial ischemia, peripheral thrombotic occlusions, coronary artery disease and acute coronary syndrome (ACS). In an embodiment, the cardiovascular disease treated or lessened by a compound of this invention is artherosclerosis. In an embodiment, the the cardiovascular disease treated or lessened by a compound of this invention is a myocardial contractility disease or disorder or an acute coronary syndrome.

In addition to the monotherapies described above, a compound of the invention is also therapeutic in combination with existing therapies directed to non-PI3K targets. In this context, a combination is defined as a fixed proportion of the compound of the invention and another non-PI3K inhibitor compound or compounds to be administered to the patient simultaneously, as in a kit, or at separate and distinct, or predetermined time periods or time intervals. The non-PI3K inhibitor compound or compounds need not be restricted to small molecular compounds such as those of this invention. The non-PI3K inhibitor compound may be a biologic such as an antibody, receptor, binding protein, lipid, sugar or the like. Furthermore, the non-PI3K inhibitor component or components of the combination may also represent energy in the form of radiation, or sources from the full range of the electromagnetic spectrum such heat, sound, X-ray or the like. Sources of irradiation, which may be externally or internally applied, include cobalt, gold, tritium, and radioisotopes capable of supplying effective translational energy for killing malignant tumors and tumorivasculary tissues.

Combinations of agents for use with the compounds of the invention in the field of cancer therapeutics, for example, include existing or novel therapeutic entities that impinge on other growth factor or proliferation pathways, activate apoptosis, inhibit cell cycle progression, inhibit angiogenesis, inhibit lymph angiogenesis, inhibit metastasis; and therapeutics of other mechanisms of action that correct or regulate cell growth to limit tumor growth and dissemination. Non limiting examples for combination therapy with the instant invention include inhibitors of mTOR and MAP kinase-dependent signaling pathways; antiproliferatives such as aromatse inhibitors; cytotoxic antiproliferatives such as topoisomerase inhibitors and tubulin inhibitors and other entities which affect cell cycle progress; inducers of apoptosis, such as ionizing radiation; inhibitors of metastasis including matrix metallo proteinase inhibitors.

By analogy, the compounds of this invention maybe combined with existing non-PI3K directed therapies for metabolic diseases, inflammatory and allergic disorders, atherosclerosis, cardiovascular disease, as described above for monotherapeutic uses. Non limiting examples include combinations of the compounds of this invention with cyclooxygenase, leukotriene inhibitors; or antibodies or binding proteins directed against the appropriate cytokine or T-cell.

In one embodiment, the novel compounds of the invention function as mono-specific inhibitors of PI3-kinase. In some embodiments, the compounds of the invention inhibit PI3K of the α, β, γ and/or δ isoforms, e.g., p110α, p110β, p110γ(p120γ), p110δ, a combination of these isoforms, or mutant or variant forms thereof. In an embodiment, one or more compounds of the invention inhibit PI3Kα, p110α, or a mutant form thereof. In an embodiment, one or more compounds of the invention inhibit PI3Kβ3, p110β, or a mutant form thereof. In an embodiment, one or more compounds of the invention inhibit PI3Kγ, p110γ(p120γ), or a mutant form thereof. In an embodiment, one or more compounds of the invention inhibit PI3Kδ, p110δ, or a mutant form thereof. In an embodiment, a compound of the invention inhibits the activity of PI3Kα, PI3Kβ, PI3Kγ, PI3Kδ, or a combination thereof. In an embodiment, a compound of the invention inhibits the activity of mutant or variant PI3Kα, PI3Kβ, PI3Kγ, PI3Kδ, or a combination thereof. In an embodiment, a compound of the invention inhibits the activity of one or more of p110α, p110β, p110γ(p120γ), p110δ, or a combination thereof In an embodiment, a compound of the invention inhibits the activity of one or more of mutant or variant p110α, p110β, p110γ(p120γ), p110δ, or a combination thereof In an embodiment, a compound of the invention inhibits PI3Kα or a mutant form thereof.

In a further embodiment, the novel compounds of the invention advantageously function as inhibitors not only of PI3K, but also as potent and selective inhibitors of other kinases, such as protein kinases, which regulate numerous biological properties, including cell growth, proliferation, differentiation, survival, migration and metabolism, and which are associated with cancers, tumors, and other diseases and pathologies. In an embodiment of this invention, the novel compounds described herein target kinases which are implicated in uncontrolled, deregulated, oncogenic, or aberrant cell growth and/or in cancers and tumors. In an embodiment, the novel compounds described herein target kinases which are implicated in stem cell development, growth and/or proliferation. Accordingly, the invention provides compounds and pharmaceutical compositions thereof, which are useful as PI3K inhibitors, e.g., a PI3Kα inhibitor, and/or protein kinase inhibitors, as well as methods for using such compounds to treat, ameliorate, reduce, eliminate, or prevent a condition, disease, or pathology, associated with abnormal or deregulated kinase activity. In some embodiments, the invention provides methods for using the compounds of the invention to treat, ameliorate, reduce the severity of, eliminate, or prevent diseases or disorders, e.g., cancers, neoplasms, tumors, inflammatory diseases, allergic diseases, etc., involving activation or activity of PDGFRα, e.g., PDGFRα(D842V), PDGFRα(V561D), PDGFRα(T674I), FLT3, e.g., FLT3(D835Y), c-KIT, e.g. c-KIT(D816V), c-KIT(V654A), EGFR, e.g. (L858R), ABL1, ABL2, ALK4, ARKS, AUR A, AXL, BLK, BMX, BRK, BTK, CAMKK2, CDK1, CDK2, CDK3, CDK5, CDK7, CK1δ, CK1ε, CK2α, CK2α2, CLK1, CLK2, CLK3, CLK4, c-MER, c-Src, DYRK1A, DYRK1B,DYRK2, DYRK3, EGFR, EPHA7, FER, FGR, FLT3, FLT4, FMS, FYN, GCK, GSK3α, GSK3β, HCK, HGK, HIPK2, HIPK3, HIPK4, IRAKI, IRAK4, ITK, KDR/VEGFR2, LCK, LOK, LYN, MELK, MLCK2, MLK1, MNK1, MNK2, MST1, MST2, mTOR, MUSK, NEK1, NEK3, PDGFRβ, PIM-1, PKCδ (delta), PKCμ (mu), PKCv (nu), PKD2, RET, RIPK2, ROS, RSK1, RSK2, RSK3, RSK4, STK33, TAK1, TAOK1, TAOK3, TRKA, TRKB, TRKC, TTK, TXK,TYK2, YES, ZAK, and or ZAP70 kinases, or mutant or variant forms of each of the foregoing. In an embodiment, the activation or activity of PDGFRα, e.g., PDGFRα(D842V), FLT3, e.g., FLT3(D835Y), c-KIT, e.g. c-KIT(D816V), EGFR, e.g. (L858R), ABL1, ABL2, ALK4, ARKS, AUR A, AXL, BLK, BMX, BRK, BTK, CAMKK2, CDK1, CDK2, CDK3, CDK5, CDK7, CK1δ, CK1ε, CK2α, CK2α2, CLK1, CLK2, CLK3, CLK4, c-MER, c-Src, DYRK1A, DYRK1B,DYRK2, DYRK3, EGFR, EPHA7, FER, FGR, FLT3, FLT4, FMS, FYN, GCK, GSK3α, GSK3β, HCK, HGK, HIPK2, HIPK3, HIPK4, IRAK1, IRAK4, ITK, KDR/VEGFR2, LCK, LOK, LYN, MELK, MLCK2, MLK1, MNK1, MNK2, MST1, MST2, mTOR, MUSK, NEK1, NEK3, PDGFRβ, PIM-1, PKCδ (delta), PKCμ (mu), PKCν (nu), PKD2, RET, RIPK2, ROS, RSK1, RSK2, RSK3, RSK4, STK33, TAK1, TAOK1, TAOK3, TRKA, TRKB, TRKC, TTK, TXK,TYK2, YES, ZAK, and or ZAP70 kinases, or mutant or variant forms thereof, comprises their abnormal or deregulated activation or activity. Compounds of the invention which may function as inhibitors of both PI3K and one or more other kinases are referred to as PI3K multiplex kinase inhibitors herein.

Because kinases are major regulators of many key cellular functions and play critical roles in a wide range of diseases and pathologies, they serve as suitable and selective targets for drugs. In general, kinases constitute the largest enzyme family, comprising about 2% of the human genome. To date, over 500 protein and lipid kinases have been identified. As targets for treating, and perhaps preventing, cancers, kinases are highly druggable molecules and can be molecularly targeted by cancer therapeutics, e.g., the compounds of this invention, to treat and prevent a variety of cancers in which the kinases, or mutant forms thereof, are overexpressed and/or active.

In accordance with this invention, the compounds of the invention serve as anti-cancer therapies to reduce, inhibit, diminish, alleviate, eradicate, eliminate, destroy and/or prevent the growth and/or recurrence of tumors, neoplasms and cancers. The novel compounds of the invention provide improved biopharmaceutical properties, isoform selectivity, potency, and pharmacokinetic profile for use in therapeutic applications. Without wishing to be bound by theory, the inventive compounds may act in one or more of the following ways to attack and, ultimately eliminate, tumor and cancer cells: by targeting angiogenesis, ultimately to starve the tumor or cancer to deprive it of its blood, oxygen and nutrient supplies; by targeting pathways for cell existence, growth, proliferation and death; by harnessing the host immune response to mount a defense against tumors, neoplasms and cancers, ultimately causing a host's rejection of the tumor, neoplasm, or cancer; and/or by targeting tumor-reinitiating cells, ultimately to eliminate tumor stem cells, which give rise to more tumor cells. The targeting of pathways intrinsic to the existence of a tumor or cancer cell frequently involves the targeting of molecules that play a role in signal transduction in a cell, or that play a role in the cell cycle, proteolysis, metabolism, or DNA repair, etc.

For the compounds of the invention that act as inhibitors of PI3-kinase and another cellular target molecule, e.g., a protein kinase, the desired level of selectivity is associated with the therapeutic area in which the compound is used as treatment. The advantages of a compound of the invention having activity against multiple targets include improving its efficacy as a drug; limiting drug resistance; broadening indications in which the compound may be effective; and having a potent and directed inhibitory effect on a selected tumor or cancer type. That an effective inhibitor of PI3K activity, particularly the inhibition of a PI3-kinase (p110α) mutation, would be of significant value, need and advantage for patients presenting with, or undergoing treatment for, tumors, neoplasms and cancers is supported by the knowledge that one or more of the molecules that play a role in the PI3K pathway may be highly mutated or aberrant in various human tumors and cancers. Illustratively and without limitation, mutational activation of the PI3K pathway is highly prevalent in human tumors and cancers. Estimates of mutation incidence are as follows: (i) brain: PI3Kα mutation (27%), PTEN deletion/mutation (40-50%); (ii) Lung: PI3Ka mutation (4%), PTEN deletion/mutation (42%); (iii) breast: PI3Kα mutation (27%), PTEN deletion/mutation (16%); (iv) gastric: PI3Kα mutation (25%); (v) liver: PI3Kα mutation (36%); ovary: PI3Kα mutation (4-12%); (vi) colon: PI3Kα mutation (32%); (vii) uterus: PI3Kα mutation (40%); (viii) prostate: PTEN deletion/mutation (40-50%).

In one embodiment, the compounds of the invention have anti-cancer/anti-tumor efficacy as a monotherapy. In another embodiment, the anti-PI3K inhibitor compounds of the invention have enhanced anti-cancer/anti-tumor efficacy in a combination therapy, e.g., when administered or provided with one or more anti-cancer drugs or molecules or other inhibitory drugs, such as, for example, chemical drugs, other small molecule compounds, or monoclonal antibodies. In an embodiment, the compounds of the invention have efficacy as inhibitors of kinases, such as MELK, which is expressed in tumor cell lines, in stem cells or progenitor cells, and in tumor stem cells or progenitor cells, e.g., brain tumor or cancer stem cells, as a monotherapy or as combination therapies. Nonlimiting examples of other cancer agents with which the compounds of the invention may be co-administered or co-provided include established anti-cancer drugs such as docetaxel, paclitaxel; VEGF inhibitors; PTEN-activating agents; and anti-oncogenic drugs. Examples of small molecule drugs for use in a combination therapy include, without limitation, Gleevec® (Novartis), which targets Bcr-abl, Kit and PDGFR for the treatment of chronic myelogenous leukemia (CML) and gastrointestinal stromal tumors (GIST); Iressa® (Astra-Zeneca), which targets EGFR for the treatment of non small cell lung carcinoma (NSCLC); Tarceva® (Genentech/OSI), which targets EGFR for the treatment of NSCLC and pancreatic cancer (PanC); Nexavar® (Bayer/Onyx), which targets VEGFR, PDGFR and Raf for the treatment of renal cell carcinoma (RCC) and hepatocellular carcinoma (HCC); Sutent® (Pfizer/Sugen), which targets VEGFR, PDGFR and KIT for the treatment of GIST and RCC; Sprycel® (Bristol-Myers Squibb), which targets Bcr-abl and Src for the treatment of CML and Ph+ acute lymphocytic leukemia (Ph+-ALL); Tykerb® (Glaxo-SmithKline), which targets EGFR and HER2 for the treatment of HER2+ breast cancer (BC); Torisel® (Wyeth), which targets mTOR for the treatment of RCC; and Tasigna® (Novartis), which targets Bcr-abl, KIT and PDGFR for the treatment of CML. Examples of monoclonal antibody drugs for use in a combination therapy include, without limitation, Herceptin® (Genentech), which targets HER2 for the treatment of HER2+ BC; Erbitux® (Imclone/BMS/Merck KGaA), which targets EGFR for the treatment of colorectal cancers (CRC) and cancers of the head and neck; and Vectibix® (Amgen (Abgenix)), which targets EGFR for the treatment of CRC.

One having skill in the art will appreciate that compounds of the invention, which are identified as PI3K inhibitors and/or protein kinase inhibitors, can be tested and validated in vivo in animal tumor models and xenograft animal models. For example, breast, colon, lung and prostate cancer xenograft xenograft models are available for assessing a compound's efficacy, alone or in combination with other small molecule or biologic drugs and compounds.

In an embodiment of the present invention, one or more compounds of the invention inhibits a protein kinase. In an embodiment, the kinase is a validated target molecule in oncology. In an embodiment of the present invention, one or more compounds of the invention targets and inhibits not only PI3K but also a protein kinase molecule, which may have implications in oncology, inflammatory disease, allergic disease, or other diseases, conditions and disorders. In an embodiment, the kinase is selected from one or more of ABL1, ABL2, ALK4, ARKS, AUR A, AXL, BLK, BMX, BRK, BTK, CAMKK2, CDK1, CDK2, CDK3, CDK5, CDK7, CK1δ, CK1ε, CK2α, CK2α2, CLK1, CLK2, CLK3, CLK4, c-MER, c-Src, DYRK1A, DYRKIB,DYRK2, DYRK3, EGFR, EPHA7, FER, FGR, FLT3, FLT4, FMS, FYN, GCK, GSK3α, GSK3β, HCK, HGK, HIPK2, HIPK3, HIPK4, IRAK1, IRAK4, ITK, KDR/VEGFR2, KIT, LCK, LOK, LYN, MELK, MLCK2, MLK1, MNK1, MNK2, MST1, MST2, mTOR, MUSK, NEK1, NEK3, PDGFRα, PDGFRβ, PIM-1, PKCδ (delta), PKCμ (mu), PKCν (nu), PKD2, RET, RIPK2, ROS, RSK1, RSK2, RSK3, RSK4, STK33, TAK1, TAOK1, TAOK3, TRKA, TRKB, TRKC, TTK, TXK,TYK2, YES, ZAK, and or ZAP70 kinases, or mutant, mutationally activated or variant forms thereof. In an embodiment, the kinase is CDK1, CDK2, PDGFRα, FLT3, MELK, GSK3α/β, TRKC, DYRK2, c-MER, CLK1, CLK4, CK2α2, or a mutant or variant form thereof. In an embodiment, the kinase is RIPK2, PIM-1, CK2α, HCK, IRAK4, MNK1 or a mutant or variant form thereof. In an embodiment, the kinase is ABL1, BMX/ETK, KIT, KIT(D816V), mTOR, ITK, MLK1, MNK2, BTK, c-SRC, FYN or a mutant or variant form thereof. In an embodiment, the kinase is CDK1, CDK7, MELK, TRKC, PIM1, or a mutant or variant form thereof. In an embodiment, the kinase is MELK. In an embodiment, the kinase is MNK, e.g., MNK1, MNK2. In an embodiment, the kinase is PDGFRα.

The above-noted kinases are involved in one or more disease indications and can serve as suitable and druggable targets whose activities and functions can be inhibited by one or more of the compounds of the present invention. More specifically, mutated PDGFRα (D842V) is involved in specific cancers, e.g., gastrointestinal soft tissue carcinoma (GIST). PDGFRα and PDGFRβ receptor tyrosine kinases (RTK) function in the regulation of cell growth and survival, as well as in angiogenesis. The PDGFRα(D842V) kinase mutant has been found to be resistant to the small molecule drugs Gleevec® and Sutent®. The FLT3, RTK, and the FLT3(D835Y) mutant kinase, are involved in haematopoiteic cancers, e.g., AML and ALL, and also function in the regulation of cell growth, survival and differentiation of hematopoietic cells in bone marrow. MELK, a novel kinase which is overexpressed in multiple cancer types, e.g., breast, brain, colon, ovary and lung cancers, is postulated to regulate cell growth and survival. MELK represents a product of a developmentally regulated gene that is highly expressed in tumor cells and in cancer stem cells. More particularly, MELK may play a role in cancers and tumors of the brain and nervous system, e.g., gliomas, glioblastomas. GSK3α/β, which is involved in cancer, Alzheimer's disease and diabetes, functions in the regulation of energy metabolism, gene transcription and cell survival. GSK3α kinase has been implicated in the production of β-amyloid plaques in Alzheimer's disease. GSK3α hyperactivity has been implicated in Type II diabetes. GSK3β kinase has been implicated in tumor cell proliferation, survival and drug resistance. The TRKC RTK (also known as NTRK3), which is involved in cancers of the brain, breast, etc., functions in the regulation of neuronal cell growth, survival and differentiation. It has been found as an oncogenic fusion protein, ETV6-NTRK3. CK2α2 kinase, which is involved in cancers of the lung and breast, is found to be overexpressed in lung and breast cancers and is implicated in the regulation of cell survival, gene transcription and DNA-break repair. DYRK2 kinase has been implicated in the regulation of cell growth and/or development. c-MER kinase, which is involved in cancer and thrombosis, has been implicated in the regulation of platelet function and retinal pigment cell phagocytosis. The aberrant expression of c-MER kinase has been observed in cases of pediatric ALL. CLK1 is a Cdc2-like kinase that has been implicated in the regulation of RNA splicing. PIM-1 kinase, which is involved in prostate cancer and lymphoma, functions in the regulation of cell proliferation and survival and can induce genomic instability. CK2α, which is involved in cancers of the lung and breast, is found to be overexpressed in lung and breast cancers and is implicated in the regulation of cell survival, gene transcription and DNA-break repair. RIPK2 kinase is involved in inflammation and in the regulation of chemokine receptor and TCR signaling and activation of NFκB. HCK is also involved in inflammation and is implicated in neutrophil migration and degranulation; this kinase may be involved in coupling Fc receptor to the activation of the respiratory burst. IRAK4 kinase is involved in inflammation and in the regulation of NFκB in response to activation of Toll-like receptors and IL-1R family members. mTOR, which is involved in cancers and transplantation, functions in the regulation of cell proliferation and survival, and in the regulation of protein synthesis and responses to hypoxia. ABL1, which is involved in cancers such as AML and CML, functions in the regulation of cell proliferation and survival and is found as oncogenic fusion proteins, e.g., Bcr-Abl, TEL-Abl. BMX/ETK, which is involved in cancer and angiogenesis, has been implicated in the regulation of angiogenesis and apoptosis by TNF and VEGF. The c-KIT RTK, and specifically mutated c-KIT(D816V) are involved in cancers such as AML and GIST, and in mastocytosis, and function in the regulation of cell growth and survival. The KIT(D816V) mutation is frequently observed in human cancers and is resistant to known drugs such as Gleevec®. ITK is involved in inflammation and in the regulation of T cell proliferation and differentiation. MLK1 kinase is known to be an upstream regulator of p38 MAP kinase signalling. MNK1 and MNK2 kinases are known to be activated by MAP kinases and are involved in the regulation of gene transcription, in particular the expression of growth promoting and and anti-apoptotic genes, by activating the eIF-4E subunit of the translation initiation complex. In particular, MNK1 is a serine/threonine kinase that acts downstream of p38 and phosphorylates eukaryotic initiation factor 4e (eIF4E), which is involved in mRNA translation and its regulation, as demonstrated by in vitro studies. BTK, which is involved in cancers such as B-cell leukemias and lymphomas and in inflammation, functions in the regulation of B cell differentiation. CDK1 and CDK2 are critical regulators of the cell cycle and regulate cell entry into, and progression through, the M- and S-phases of the cell cycle. Accordingly, it will be appreciated from the foregoing that there is a serious need for new compounds that target these kinases and mutant kinases and inhibit their respective functions and activities, for the treatment of cancers, such as GIST, cancers of the brain and nervous system, breast cancer, brain cancer, lung cancer, and ovarian cancer, and in the treatment of inflammatory diseases, allergy diseases and other diseases, disorders, pathologies and conditions.

In an embodiment, one or more compounds of the invention inhibits PI3K and MELK (maternal embryonic leucine zipper kinase), which is a member of the Snfl/AMPK-related ser/thr kinase family. MELK is expressed mainly during embryonic development and is found in low abundance in adult tissues; however, this kinase is overexpressed in multiple cancers, in particular, in 96% of brain tumors/cancers, in 23% of lung tumors/cancers, in 92% of breast tumors/cancers, in 13% of ovary tumors/cancers and in 96% of colon tumors/cancers. In brain cancer stem cells, the overexpression of MELK correlates with malignancy grade and survival in GBM patients. This kinase is postulated to act as a critical regulator of cell cycle progression and apoptosis. Thus, a compound of this invention that inhibits the activity of MELK advantageously serves as a drug that can affect oncogenic function and treat tumors and cancers in which this kinase activity results in a dysregulation of cell growth. In an embodiment, one or more compounds of the invention inhibits PI3Kα and MELK.

Human cell models exist that relate to particular tumor types in which MELK activity and/or overexpression is associated and for which an oncogenic function can be assessed following treatment with a given drug, compound or agent that targets MELK. In this way, candidate compounds, including compounds of the present invention, can be tested for their activity against MELK. Additionally, siRNA that inhibits MELK activity has been used in validating MELK as an oncogenic target in a number of tumor types, for example, tumors of the breast, colon, brain, pancreas and cervix, cells transformed by tumor virus, and brain cancer stem cells, in which oncogenic function associated with MELK activity is assessed. MELK siRNA studies provide tools with which to assess the inhibitory activity of the compounds of the invention against an oncogenic function attributed to MELK activity in human tumor cell models in an assayable format. Illustratively, human cell models of breast cancers/tumors include T47D, MCF-7 and BT-549, with growth as assessable oncogenic function; a human cell model of colon cancer/tumor includes HCT-116, with growth as assessable oncogenic function; human cell models of brain cancers/tumors include Daoy, T98G, U-87MG, GBM1600 derived from primary human tumors and GNS1, GNS2, GNS3 and GNS4 derived from primary human tumors, with growth and survival as assessable oncogenic function; a human cell model of pancreatic cancer/tumor includes PANC-1, with growth as assessable oncogenic function; a human cell model of cervical cancer/tumor includes HeLa, with growth as assessable oncogenic function; a human cell model of tumor virus transformed cells includes SVT2 (mouse 3T3/SV40), with tumor growth in mice as assessable oncogenic function; a human cell model of brain cancer stem cells includes GNS4, with cell growth and survival as assessable oncogenic function.

An ability of the compounds of the invention to target and inhibit the activities of PI3K and MELK, of PI3K, or of MELK, provides a profound advantage for the use of these compounds to treat various cancers, tumors, diseases and conditions in which the activity of these kinases, or mutated forms thereof, are associated with aberrant cell growth and/or survival, leading to debilitating disease and pathologies. As an example, mutation of PI3K and overexpression of MELK have been reported in several cancer and tumor types in which dysregulation of one or more molecules in cellular pathways and processes have been implicated in causing, or being associated with, resulting diseases and cancers. More specifically, it has been found that 27% of brain cancers harbor a p110α mutation and 96% of such cancers show overexpression of MELK; 4% of lung cancers harbor a p110α mutation and 23% of such cancers show overexpression of MELK; 27% of breast cancers harbor a p110α mutation and 92% of such cancers show overexpression of MELK; 32% of colon cancers harbor a p110α mutation and 96% of such cancers show overexpression of MELK; and 4-12% of ovarian cancers harbor a p110α mutation and 13% of such cancers show overexpression of MELK. Additionally, 25% of gastric cancers, 36% of liver cancers and 40% of uterine cancers harbor a p110a mutation. Further, 40-50% of brain cancers, 16% of breast cancers, 42% of lung cancers and 40-50% of prostate cancers have been found to have a PTEN deletion/mutation. Thus, targeting these (and other) cancer- and disease-associated kinases as provided by compounds such as those of the present invention affords new treatments and drug therapies for patients afflicted with a wide range of cancers and tumors.

In another embodiment, one or more compounds of the invention inhibits PI3K and PDGFRα (D842V). The D842V mutation has been observed to be the most prevalent PDGFRα-activating mutation in GIST, a soft tissue carcinoma of the GI tract, which represents 1-3% of all GI cancers and affects approximately 5000 patients per year in the United States. GIST occurs across all geographic regions, ethnic groups and genders and is particularly diagnosed in patients over 50 years of age. Because the therapeutic options for treating GIST are neither universally successful nor optimal, surgery is the standard of care for primary GIST disease. However, surgery is not always effective. In addition, GIST is insensitive to radiation therapy and is resistant to conventional chemotherapy. Further, GIST carrying the PDGFRα-activating mutation has shown primary and secondary resistance following treatment with Gleevec® (Imatinib mesylate), and is insensitive to Sutent® and Tarsigna®.

In another embodiment, one or more compounds of the invention inhibit PI3K and MNK1 and/or MNK2. The MNK1 and MNK2 kinases are regulated via the ERK1/2 and p38^{MAPK} pathways, positioning these kinases on stress and proliferative signaling pathways. MNK1 and MNK2 regulate protein translation through the elongation initiation factor 4E (eIF-4E), a component of the translation initiation complex. eIF-4E is overexpressed in human cancers including breast, colon and head-and-neck cancers. Overexpression of MNK1 and eIF-4E induces tumors. eIF-4E regulates genes containing long, GC-rich 5′-UTR sequences that are associated with cell proliferation and survival, including pro-proliferative factors, cyclin D1, VEGF and FGF, and anti-apoptotic factors survivin, c-IAP, Bcl-_{XL}. Protein translation is a critical process in tumorigenesis and cancer progression. eIF-4E is regulated by the mTOR substrate, 4E-binding proteins (4E-BPs), 4E-BP1 is limited in its level of expression, and therefore the overexpression of eIF-4E is sufficient to overcome 4E-BP1 mediated silencing of eIF-4E. Agents that target protein translation through blockade of PI3K and/or mTOR signaling alone would be expected to be less efficacious under conditions of eIF-4E overexpression. Agents that inhibit PI3K and MNK1 and/or MNK2 would therefore be expected to be more efficacious in tumors and cancers where eIF4E is overexpressed. At present, there are no medically approved therapies that target MNK1 and/or MNK2. Accordingly, it will be appreciated that there are significant benefits and as-yet unmet needs for compounds of the present invention that can target and inhibit a kinase associated with oncology, inflammation, and/or other diseases. It will be further appreciated that there are significant benefits and as-yet unmet needs for a compound of the present invention that can target and inhibit both PI3-kinase and another kinase that is associated with oncology, inflammation, and/or other diseases. In an embodiment such other kinase is MELK. In an embodiment, such other kinase is PDGFRα (D842V). In an embodiment, such other kinase is MNK1 and/or MNK2. In various embodiments, a pyrazoloquinoline of Formula (VI) according to the present invention inhibits PI3Kα and MELK, or PI3K and PDGFRα(D842V), or PI3K and MNK1 and/or MNK2.

In another embodiment, the invention encompasses a method of inhibiting PI3K activity in a cell or in a biological sample, comprising contacting the cell or biological sample with a compound or composition of the invention. In another embodiment, the invention encompasses a method of inhibiting a specific protein kinase activity in a cell or in a biological sample, comprising contacting the cell or biological sample with a compound or composition of the invention. In an embodiment, the protein kinase is one or more one or more of, but not limited to, ABL1, ABL2, ALK4, ARKS, AUR A, AXL, BLK, BMX, BRK, BTK, CAMKK2, CDK1, CDK2, CDK3, CDK5, CDK7, CK1δ, CK1ε, CK2α, CK2α2, CLK1, CLK2, CLK3, CLK4, c-MER, c-Src, DYRK1A, DYRK1B,DYRK2, DYRK3, EGFR, EPHA7, FER, FGR, FLT3, FLT4, FMS, FYN, GCK, GSK3α, GSK3β, HCK, HGK, HIPK2, HIPK3, HIPK4, IRAK1, IRAK4, ITK, KDR/VEGFR2, KIT, LCK, LOK, LYN, MELK, MLCK2, MLK1, MNK1, MNK2, MST1, MST2, mTOR, MUSK, NEK1, NEK3, PDGFRα, PDGFRβ, PIM-1, PKCδ (delta), PKCμ (mu), PKCν (nu), PKD2, RET, RIPK2, ROS, RSK1, RSK2, RSK3, RSK4, STK33, TAK1, TAOK1, TAOK3, TRKA, TRKB, TRKC, TTK, TXK,TYK2, YES, ZAK, and or ZAP70 kinases, or mutant, mutationally activated or variant forms thereof. In an embodiment, the kinase is RIPK2, PIM-1, CK2α, HCK, IRAK4, or a mutant or variant form thereof. In an embodiment, the kinase is ABL1, BMX/ETK, c-KIT, mTOR, ITK, MLK1, MNK1, MNK2, BTK, or a mutant or variant form thereof. In an embodiment, the kinase is MELK, TRKc, PDGFRα, PIM1, or a mutant or variant form thereof. In an embodiment, the kinase is MELK. In an embodiment, the kinase is PDGFRα(D842V). In an embodiment, the kinase is MNK 1 or MNK2.

A biological sample refers to an in vitro or ex vivo sample and includes, without limitation, cell cultures or extracts thereof; cell, tissue or organ samples, or extracts thereof; biopsied material obtained from a subject, i.e., an animal or mammalian subject, including humans, or extracts thereof; and blood, plasma, serum, urine, saliva, feces, semen, tears, body cavity lavage material, or other body fluids or extracts thereof.

In another embodiment, one or more of the compounds of the present invention, or pharmaceutically acceptable compositions containing such compounds, are employed for coating or lining an implantable medical device, e.g., stents, catheters, grafts, vascular grafts, prostheses and artificial valves. As will be appreciated by one having skill in the art, vascular grafts have been used to overcome restenosis, or a re-narrowing of a vessel wall following injury or surgery. In some patients, the implantation of a stent or another type of implantable device may be associated with a risk of clot formation (embolism) or platelet activation. To overcome or mitigate this risk, the stent or device can be coated (pre-coated) with a compound of the invention or a pharmaceutically acceptable composition thereof. In accordance with the invention, such coating (or pre-coating) may reduce or prevent inflammation reactions or undesirable cell proliferation following implantation.

General description of suitable coatings and coated implantable devices may be found in U.S. Pat. Nos. 6,099,562; 5,886,026 and 5,304,121. The coatings are typically biocompatible polymeric materials such as a hydrogel polymer, polymethyldisiloxane, polycaprolactone, polyethylene glycol, polylactic acid, ethylene vinyl acetate and mixtures or combinations thereof Optionally, the coatings may be covered with a suitable topcoat of a material such as fluorosilicone, polysaccharides, polyethylene glycol, phospholipid, or a combination thereof, to impart controlled release characteristics for the coated compounds or compositions. An implantable medical device coated or lined with a compound or composition according to the present invention is a further embodiment embraced by the invention. Additionally, the compounds may be coated on an implantable medical device through the use of beads or particles or through co-formulation with a polymer or other molecule to provide a drug depot, which allows the compound (drug) to be released over a longer time period relative to the administration of an aqueous formulation of the compound (drug).

Although it is understood that mutational activation of PI3K and/or select receptors that signal through PI3K can sensitize human tumor cells to PI3K inhibitors, the inhibition of PI3K activity in tumors may be affected by the presence of several oncogenes, including those that encode kinases, non-kinases, transcription factors, and GTPases, e.g., Src (kinase), Ras (GTPase), Cyclin B (non-kinase), and Myc (transcription), which can act as “resistance factors” leading to resistance to PI3K inhibition. Although the PI3K pathway is the most mutated pathway in human cancers, many of these “resistance factors” are also highly prevalent in human cancers, and may play a role in a large subset of patients who are poor or non-responders to PI3K selective therapies. This is supported by several preclinical studies demonstrating the lack of efficacy of PI3K inhibitors in tumors harboring mutated Ki-Ras. Because functional redundancy exists between molecules or factors in different pathways that regulate cell growth, survival, protein translation, etc, inhibition of the molecules or factors in one pathway can be overcome by the upregulation or substitution of those in another pathway. In addition, preclinical evidence has demonstrated that PI3K-selective inhibitors (i.e. inhibitors of PI3K family members only) are, generally, cytostatic agents, and that cancer cells and tumors regrow after drug removal. The present invention provides unique inhibitors that not only inhibit the PI3K pathway but also additional, complementary or parallel pathways (e.g. Ras-MAPK) or components of those pathways, e.g. MNK1/2, so as to minimize or eliminate the potential for pathway redundancy and PI3K inhibitor resistance. Accordingly, the compounds of the invention serve as targeted therapies or signal transduction inhibitors, including PI3K inhibitors, that not only block tumor cell proliferation and tumor growth but are able to induce tumor cell death.

In accordance with the invention, the PI3K and kinase signaling pathways offer various targets for therapeutic intervention by inhibitor compounds provided herein. As described, compounds of the invention inhibit PI3K, e.g., PI3K-α, activity alone, or in combination with one or more cellular kinases. An aspect of the therapeutic potential for a specific inhibitor of one or more targets of PI3K signaling is the ability to identify predictors, e.g., positive or negative predictors, of response to such an inhibitor. Such an ability allows the identification of those individuals afflicted with one or more cancers or tumors who are the most likely to receive the maximum therapeutic benefit from an inhibitor used as a treatment drug can be identified. For example, mutant PI3K-α (i.e., p110α) and loss of PTEN activity can be sufficient, but not necessary, predictors of sensitivity to an inhibitor in the presence of wild-type Ras, while, by contrast, mutant, oncogenic Ras serves as a clearer predictor of resistance to the inhibitor. (N. T. Ihle et al., 2009, Cancer Res., 69(1): 143-150). This may occur even in tumors having coexisting mutations in PI3K-α, since mutant active Ras is able to mediate resistance through its ability to utilize multiple pathways for tumorigenesis. (Ibid.) Accordingly, mutant oncogenic Ras may be considered to be a primary determinant of resistance to the anti-tumor activity of a therapeutic PI3K-a inhibitor compound or drug. The ability of a PI3K inhibitor to inhibit PI3K activity and mutant Ras activity would allow treatment for those patients who are identified as having tumors containing mutant Ras. In an embodiment, the invention provides compounds that inhibit PI3K-α in cells having mutated Ras.

Ras (e.g., Ki-Ras, H-Ras and N-Ras) mutations occur in a variety of human cancers and tumors and in a number of cancers and tumors which also have mutations in PI3K-α (i.e., p110α). For example, 50% of thyroid cancers have been found to have mutations in Ras. In addition, 10% of breast cancers, 90% of pancreatic cancers, 40-60% of colon cancers, 25% of cervical cancers, 30% of lung cancers, 30% of blood cancers, e.g., leukemia, etc., 50% of ovarian cancers and 30% of skin cancers have been shown to have Ras mutations. In many of the same cancers and tumors, p110a mutations are common. For example, p110α mutations are found in 27% of brain cancers and tumors, 26% of breast cancers and tumors, 25% of gastric cancers and tumors, 36% of liver cancers and tumors, 32% of colon cancers and tumors, 40% of uterus cancer and tumors, 4% of lung cancers and tumors and 4-12% of ovarian cancers and tumors. Thus, illustratively and without limitation, it can be seen that a high percentage of cancers and tumors of the breast, colon, lung and ovary show mutations in both p110α and Ras. Moreover, in many of the foregoing cancers and tumors, PTEN deletions or mutations are found. For example, PTEN deletions or mutations have been found in 40-50% of brain cancers and tumors, in 16% of breast cancers and tumors, in 42% of lung cancers and tumors and in 40-50% of prostate cancers and tumors.

It has been reported that those cancers and tumors having a mutated Ras protein are frequently resistant to inhibitors of PI3K, particularly, PI3K-α. Thus, mutation in Ras is a potential indicator of resistance to PI3K inhibition in cancers and tumors to be treated. Accordingly, an inhibitor of PI3K, particularly PI3Kα, which is effective in treating cancers and tumors which also have mutated Ras is a much needed and unique oncogenic therapeutic. The present invention provides such inhibitors, namely, compounds of the invention show effective inhibition of PI3K-α in human cancer and tumor xenograft cell lines of various tissue origins having mutations in Ras. For example, PI3K inhibition resistant human tumor xenografts from a variety of commercially-available histotypes, such as breast, gliobastoma, medulloblastoma, melanoma, prostate, colorectal, non-small cell lung carcinoma (NSCLC), myeloma, pancreas and bladder, have been identified. Examples of such cell lines include, but are not limited to, MCF-7, U-87 MG, T98G, Daoy, A375, Colo829, G361, LNCaP C4-2, HCT116, HCT15, RKO, Colo205, T84, NCI-H1299, A549, NCI-H69, NCI-H460, RPMI-8226A, PANC-1, MiaPaCa-2, Capan-2, AsPC-1, PL45 and T24. A number of these cell lines contain mutated Ras (e.g., mutated Ki-Ras or H-Ras); some contain additional mutations, such as in MAP kinase or B-Raf (e.g., B-Raf^{V600E}). As described in Example 8, compounds of the invention inhibit proliferation of human xenograft cell lines that are resistant to PI3K inhibition.

The inhibitors of the invention can induce apoptosis, i.e., are pro-apoptotic, in oncogenic cells having oncogenic potential in in vitro assays. In an embodiment, the inhibitors induce early apoptosis. In an embodiment, the inhibitors induce late apoptosis. To assess the pro-apoptotic activity of compounds of the invention, HCT116 cells containing mutated Ras, (Ki-Ras^{G13D}) and mutated PI3K-α (PI3K-α^{H1047R}) were incubated with a Pyrazoloquinoline compound of the invention at concentrations of 0.5, 1.0 and 5.0 μM for 16 hours. Cell lysates were prepared and the relevant indicators of apoptosis were analyzed—active caspase 3 in the case of early apoptosis and cleaved PARP in the case of late apoptosis. At the 1.0 and 5.0 μM concentration, a representative pyrazoloquinoline compound of the invention induced both early and late cellular apoptosis.

The compounds of the invention inhibit the proliferation of tumor cells from various tissue sources as demonstrated in cell based assays in vitro. A representative PI3K-α inhibitor compound of the invention (PI3K-α inhibition: ˜3 nM) was found to inhibit proliferation of glioblastoma, colorectal cancer, breast, pancreas, and NSCLC cells at EC50 values ranging from about 23 nM to about 248 nM. Other compounds of the invention inhibited these cells at EC50 values ranging from about 10 nM to about 600 nM.

The invention further provides compounds that demonstrate anti-proliferative and apoptotic activity. In an embodiment, the compounds have cytotoxic activity in cells harboring Ras mutations, as demonstrated in Ras mutated cell lines. In an embodiment, compounds of the invention block MNK-eIF4E signaling (protein translation). In an embodiment, compounds of the invention demonstrate cytostatic activity. In an embodiment, compounds of the invention demonstrate cytotoxic activity and induce cell death. In an embodiment, compounds of the invention induce caspase activity, which leads to cell death, in tumors harboring mutations that confer resistance to PI3K-selective inhibitors. Examples of such tumors include, without limitation, those with Ras mutations, those with Src mutations, those with Myc overexpression, those with Cyclin B overexpression, or those with a combination thereof. In an embodiment, compounds of the invention target and inhibit protein translation downstream of AKT-mTOR, i.e., pathways involving MNK-eIF4E or MAPK-RSK.

### EXAMPLE A

### Assays for Determining Activity of PI3K Compounds of the Invention

Culture media and experimental reagents: Dulbecco's Modified Eagle's Medium (DMEM), Medium 200, fetal bovine serum (FBS) and Low Serum Growth Supplement (LSGS), and antibiotics (penicillin/streptomycin) were purchased from Invitrogen. Bovine serum albumin (BSA), ultrapure ATP and dimethylsulfoxide (DMSO) were purchased from Sigma-Aldrich. Insulin-like growth factor-1 (IGF-1) was purchased from EMD Calbiochem. Recombinat kinases (PI3Kα, PI3Kβ, PI3Kδ and PI3Kγ, mutant PI3Kα(H1047R), mutant PI3Kα(E545K) and MELK) and ZIPtide peptide substrate were purchased from Invitrogen or Millipore. Vascular endothelial cell growth factor (VEGF₁₋₁₆₅) was purchased from R&D Systems. All primary antibodies were purchased from Cell Signaling Technology. Horseradish peroxidase (HRP) conjugated antibodies were purchased from GE Healthcare. Fluorophore-labeled IRDye 800CW and IRDye 680 detection antibodies and Odyssey blocking buffer were purchased from LI-COR Inc. Enhanced chemiluminescence (ECL) reagents were purchased from Pierce. All reference kinase inhibitors were purchased from commercial sources.

Cell culture: All human tumor cell lines were purchased from ATCC (American Type Culture Collection, P.O. Box 1549, Manassas, Va. 20108 USA) and were maintained in DMEM supplemented with 10% (v/v) fetal bovine serum (FBS) and antibiotics (complete medium). Human umbilical vein endothelial cells (HUVEC) and human microvascular endothelial cells (HMVEC) were purchased from Lonza and maintained in Medium 200 supplemented with LSGS and antibiotics (EC medium). All cell lines were maintained at 37° C. in a 5% CO₂ atmosphere.

Compound Preparation: KinaseGLO assay: 30× stocks of inhibitors were prepared in DMSO. A 10 mM master stock was prepared and all other stocks were generated by serial dilution of this master stock in DMSO. The final concentration of DMSO in the kinase reaction was 3.3% (v/v).

Compound Preparation: Cell-based assays: 1000× stocks of inhibitors were prepared in DMSO. A 10 mM master stock was prepared and all other stocks were generated by serial dilution of the master stock in DMSO. The final concentration of DMSO in cell-based assays was 0.1% (v/v).

PI3-Kinase Assay: The KinaseGLO® Luminescent Kinase Assay (Promega) is a luciferin-luciferase based luminescent assay that utilizes a proprietary thermostable luciferase to quantify the ATP remaining in a solution after a kinase reaction. The luminescent signal generated using KinaseGLO® directly correlates with ATP concentration, and therefore inversely correlates with kinase activity.

PI3K enzyme sample was thawed and diluted to 100 ng/μL in enzyme dilution buffer (50 mM HEPES, pH7.5, 3.0 mM MgCl₂, 1.0 mM EGTA) and stored on ice. PI3Kα enzyme was diluted to a final concentration of 1 ng/μL in reaction buffer (for PI3Kα; 50 mM HEPES, pH7.5, 100 mM NaCl, 2.0 mM DTT, 3.0 mM MgCl₂, 1.0 mM EGTA, 10 μM ATP) and gently mixed. 25 μL of this kinase reaction was then aliquoted into test wells in a Microfluor2 white 96-well plate (Thermo Scientific). As a preincubation step, 1 μl of 30× compound dilutions was added to the appropriate test wells to generate a ten point dose-response ranging from 0.0 μM-10 μM inhibitor. The enzyme-inhibitor mixture was then incubated for 30 minutes at 4° C. After this preincubation, 4.0 μL of 200 μM SC-PI stock (50 μM final concentration) was added to each test well to initiate the kinase reaction. Reactions were incubated at room temperature for 2 hours with moderate shaking. The KinaseGLO® luminescent reagent (Promega) was prepared according to the manufacturer's instructions. Both the KinaseGLO® buffer and substrate were allowed to thaw and equilibrate at room temperature for two hours prior to mixing and use. The PI3-kinase reaction was terminated by the addition of 30 μL of reconstituted KinaseGLO® reagent to each reaction well and then incubated in the dark for 10 minutes at room temperature. Luminescent signal was detected using a SpectraMax M5 (Molecular Devices). Maximal luminescence (no PI3K activity/maximal PI3K inhibition) was determined from reaction buffer plus SC-PI substrate only controls. The minimal signal (maximal PI3K activity) was determined by the 0.0 μM compound (DMSO only) control. IC₅₀ values were calculated via non-linear regression using GraphPad Prism data analysis software.

MELK kinase assay: The ability of compounds to inhibit MELK was also determined using the KinaseGLO assay. Briefly, MELK enzymes was thawed and diluted to a final concentration of 2.64 ng/μL in reaction buffer (50 mM HEPES, pH7.5, 100 mM NaCl, 2.0 mM DTT, 3.0 mM MgCl₂, 1.0 mM EGTA, 10 μM ATP). 19 μl of this kinase reaction mixture was aliquoted into test wells (50 ng MELK/well) in a Microfluor2 white 96-well plate (Thermo Scientific). As a preincubation step, 1 μl of 30× compound dilutions was added to the appropriate test wells to generate a ten point dose-response ranging from 0.0 μM-10 μM inhibitor, and incubated for 15 minutes at 4° C. The reaction was started by addition of 10 μL of 150 μM ZIPtide peptide substrate resuspended in reaction buffer (50 μM final concentration of ZIPtide). Reactions were incubated at room temperature for 2 hours prior to addition of 30 μL of KinaseGLO reagent and development as described. Maximal luminescence (no MELK activity/maximal MELK inhibition) was determined from reaction buffer plus peptide substrate only controls. The minimal signal (maximal MELK activity) was determined by the 0.0 μM compound (DMSO only) control. IC₅₀ values were calculated via non-linear regression using GraphPad Prism data analysis software.

In-cell western blot (cytoblot) mechanistic assay; Measurement of AKT phosphorylation: The in-cell western blot assay is a quantitative 96-well immunocytochemistry assay using fixed cells. Phosphorylated AKT is detected and quantified via either enhanced chemiluminescence (ECL) or near infra-red (LI-COR) methods. Log-phase cells (10,000 A549, RKO or 30,000 U-87 MG) were plated in collagen coated, 96 well plates (Nunc) in 200 μL of growth medium (10% FBS/DMEM) and allowed to attach overnight at 37° C. in 5% CO₂ incubator. Black 96 well plates were used for ECL assays. White, clear bottomed 96 well plates were used for LI-COR assays. The following day the growth medium was removed. For experiments using U-87 MG cells and RKO cells, the growth medium was replaced with 200 μL of fresh growth medium containing inhibitors dissolved in DMSO. PI3K inhibitors were dosed using half-log dilutions to generate an eight-point dose-response curve from 0.0 μM-10 μM for EC₅₀ determinations, and the final concentration of DMSO in the test wells was 0.1% (v/v). The inhibitor treated cells were then incubated for 1 hour at 37° C. For A549 cells, the growth medium was removed and replaced with starvation-medium (DMEM) containing 0.1% BSA and the cells were incubated for an additional 18 hours at 37° C. The starvation medium was replaced with 200 μL of fresh starvation medium containing inhibitors dosed using half-log dilutions to generate an eight-point dose-response curve from 0.0 μM-10 μM. The cells were then incubated with test compound for 1 hour at 37° C. After this incubation, the serum-starved A549 cells were stimulated for 10 minutes with 50 ng/ml IGF-1 to induce PI3K activation and AKT phosphorylation.

After the inhibitor incubation phase (with or without IGF-1 stimulation), the medium was aspirated, the cells washed once with Tris-Buffered Saline (TBS) and then fixed in the wells by addition of 200 μL of cold 3.7% (v/v) formaldehyde diluted in TBS to test wells and incubated for 15 minutes at 4° C. The formaldehyde was removed and the cells permeabilized by the addition of 50 μL of methanol (at −20° C.) and incubated for 5 minutes: (ii) Enhanced Chemiluminescence (ECL) Detection: The methanol was removed, and the test wells were blocked with 200 μL of 1% (w/v) BSA in TBS to block non-specific antibody binding sites. The plates were then incubated for 30-60 minutes at room temperature. After removal of the blocking buffer, 50 μL of 1:250 diluted p-(S473)AKT or p-(T308)AKT antibodies (Cell Signaling Technology) in 0.1% (w/v) BSA in TBS, was added to test wells and the plate were incubated at room temperature for 1 hour or overnight at 4° C. with gentle rotation. Plates were then washed 3 times with cold TBS containing 0.05% (v/v) Tween 20 (TBS-T). Next, 100 μL of a 1:250 dilution of horseradish peroxidase (HRP)-conjugated antibody diluted in TBS-T was added to test wells and the plate was incubated for 1 hour at room temperature. The wells were then was washed four times with ice-cold TBS-T. Phospho-AKT luminescent signal was detected after a 1 minute incubation of test wells with 100 μL of ECL reagent using a SpectraMax M5 luminometer. (ii) Near-infrared fluorescence detection (LI-COR): After removal of the methanol, wells were blocked with 200 μL of LI-COR blocking buffer for 1 hour at room temperature. Test wells were then incubated with 50 μL of phospho-AKT antibody diluted 1:500 to 1:1000 in LI-COR blocking buffer and incubated for 1-18 hours with gentle shaking. Wells were washed four times with TBS containing 0.05% Tween 20 (TBS-T). 50 μL of LI-COR IRDye-conjugated detection antibody, diluted 1:500 in LI-COR blocking buffer, was added to test wells and incubated in the dark at room temperature for 1 hour. Test wells were washed four times in the dark with TBST and the plates then scanned on a LI-COR Odyssey near-infra red fluorescent imager (LI-COR Inc.) to detect and quantitate AKT phosphorylation. EC₅₀ values for both assays were calculated via non-linear regression using GraphPad Prism data analysis software.

CellTiter-GLO® Cell Proliferation Assay: CellTiter-GLO® (Promega) quantifies cell number as a measure of the ATP in a cell culture. 1,000-10,000 human tumor cells (of multiple histotypes) were plated in 100 μL of growth medium (10% (v/v) FBS in DMEM) in white, collagen coated 96-well plates (Biocoat) and allowed to adhere overnight at 37° C. in a 5% CO₂ incubator. All test inhibitors were prepared in DMSO. For EC₅₀ determinations, 1000× inhibitor stocks were prepared from a 10 mM master stock by serial dilution in DMSO and subsequently diluted in fresh growth medium to the desired inhibitor concentration. The growth medium was removed by aspiration and replaced with fresh medium containing inhibitors. The final concentration of DMSO in a test well was 0.1% (v/v). For EC₅₀ determinations, eight-point dose responses ranging from 0.0 μM to 10 μM inhibitor were used. Cells were then incubated with inhibitors for 72 hours at 37° C. in a 5% CO₂ incubator. Control cells (0.1% DMSO only) were included for time 0 hours and at 72 hours to determine the starting cell number and cell number after 72 hours of proliferation.

After 72 hours, the plates were removed from the incubator and equilibrated to room temperature for 30 minutes. CellTiter-GLO® reagent (Promega) was prepared according to the manufacturers instructions. 100 μL of freshly prepared CellTiter-GLO® reagent was added to each test well and the contents mixed at room temperature for 2 minutes on an orbital shaker. Plates were incubated in the dark for 10 minutes at room temperature before reading luminescence on a SpectraMax M5 luminometer. Results are expressed as % inhibition relative to control cells (DMSO only). EC₅₀ values were calculated via non-linear regression using GraphPad Prism data analysis software.

Endothelial cell (EC) proliferation assay: To evaluate the anti-angiogenic activity of the PI3K inhibitor compounds of the invention, compounds were assayed in a VEGF-induced endothelial cell proliferation assay. HUVEC cells were seeded at 10,000 cells per well in clear, gelatin-coated 96 well microplates and allowed to attach overnight. The following day, the EC medium was removed and the cells incubated in fresh EC medium containing increasing concentrations of the PI3K inhibitor compounds (from 0.003 μM to 10.0 μM in half-log dilutions). Cells were then stimulated with 50 ng/mL of VEGF₁₋₁₆₅ in the presence of bromodeoxyuridine (BrdU) for 24 hours. Cell proliferation was detected using the Cell Proliferation (BrdU) ELISA assay, according to the manufacturer's instructions (Roche), and measured on a Spectramax M5 multi-detection microplate reader (Molecular Devices Corp.). EC₅₀ values were calculated via non-linear regression using GraphPad Prism data analysis software.

Caspase-GLO 3/7 Assay: To determine the ability of the PI3K inhibitor compounds of the invention to induce programmed cell death (apoptosis), caspase activity was monitored as an indicator of apoptosis. Human HCT116 colorectal carcinoma and PANC-1 pancreatic carcinoma cells were plated at 2000-8000 cells per well in white 96-well microplates and allowed to attach overnight. The following day the medium was removed by aspiration and the cells were washed once with 200 μL of DMEM containing 0.5% (v/v) FBS (assay medium). The cells were then incubated in 100 μL of assay medium containing increasing concentrations of the PI3K inhibitor compounds (from 0.003 μM to 10.0 μM in half-log dilutions) for 24 to 72 hours. Caspase activity was detected using the Caspase-GLO 3/7 assay (Promega) according to the manufacturer's instructions and measured on a Spectramax M5 multi-detection microplate reader (Molecular Devices Corp.). EC₅₀ values were calculated via non-linear regression using GraphPad Prism data analysis software.

Western blot analysis: 10 μg-25 μg of cell protein was resolved by SDS-PAGE and transferred to nitrocellulose or PVDF membranes. Membranes were blocked with Odyssey Blocking Buffer and probed with primary antibodies diluted 1:200-1:000 in Odyssey blocking buffer for up to 16 hours at 4° C. After washing, proteins of interest were detected signal via IRDye-labeled antibodies diluted 1:500 in Odyssey blocking buffer for one hour at room temperature and visualized on an Odyssey near-infra red fluorescent imager (LI-COR Inc.).

Kinase Profiling: The inhibitory activity of the PI3K inhibitor compounds of the invention against 250 protein kinases was determined via the radiometric HotSpot Kinase Profiling (Reaction Biology Corporation, Malvern, Pa.). Compounds were profiled at a final concentration of 1 μM compound against recombinant protein kinases in the presence of 10 μM ATP and data were expressed as percent inhibition. IC50 and/or percent inhibition at 1 μM compound values were determined for select compounds against a subset of kinases (MELK, MNK1, MNK2, mTOR, PIM-1. TRKC, and PDGFRα(D842V)).

### EXAMPLE B

### Activities of Compounds of the Invention

Various embodiments of a pyrazoloquinoline compound of the invention may inhibit PI3Kα at an IC50 value of <100 nM, or <500 nM, or <2000 nM. An embodiment of a pyrazoloquinoline compound of the invention may inhibit PI3Kα at an IC₅₀ value of <0.001-0.850 μM. An embodiment of a pyrazoloquinoline compound of the invention may inhibit PI3Kα at an IC₅₀ value of 0.006-0.500 μM. Another embodiment of a pyrazoloquinoline compound of the invention may inhibit PI3Kα at an IC₅₀ value of 0.005-0.100 μM. An embodiment of a pyrazoloquinoline compound of the invention may inhibit PI3Kα at an IC₅₀ value of 0.008-0.060 μM. An embodiment of a pyrazoloquinoline compound of the invention may inhibit PI3Kα at an IC₅₀ value of 0.010-0.050 μM. An embodiment of a pyrazoloquinoline compound of the invention was found to inhibit PI3Kα at an IC₅₀ value of 0.008 μM, 0.009 μM, 0.010 μM, 0.011 μM, 0.036 μM, 0.046 μM, 0.061 μM, 0.177 μM, or 0.467 μM. In another embodiment, a pyrazoloquinoline compound of the invention may inhibit PI3Kα(E545K) at an IC₅₀ value of from 0.005-0.050 μM. An embodiment of a pyrazoloquinoline compound of the invention may inhibit PI3Kα(E545K) at an IC₅₀ value of 0.009 μM, 0.031 μM, or 0.038 μM.

An embodiment of a pyrazoloquinoline compound of the invention may inhibit PI3Kα(H1047R) at an IC₅₀ value of from 0.005-0.100 μM. In another embodiment, a pyrazoloquinoline compound of the invention may inhibit PI3Kα(H1047R) at an IC₅₀ value of from 0.010-0.060 μM. In an embodiment, a pyrazoloquinoline compound of the invention may inhibit PI3Kα(H1047R) at an IC₅₀ value of from 0.005-0.100 μM. An embodiment of a pyrazoloquinoline compound of the invention may inhibit PI3Kα(H1047R) at an IC₅₀ value of from 0.015-0.060 μM. In an embodiment, a pyrazoloquinoline compound of the invention may inhibit PI3Kα(H1047R) at an IC₅₀ value of 0.016 μM, 0.034 μM, or 0.058 μM. An embodimentof a pyrazoloquinoline compound of the invention may inhibit PI3Kβ at an IC₅₀ value of from 0.015-0.800 μM. Another embodiment of a pyrazoloquinoline compound of the invention may inhibit PI3Kβ at an IC₅₀ value of 0.030-0.750 μM. In an embodiment, a pyrazoloquinoline compound of the invention may inhibit PI3Kβ at an IC₅₀ value of 0.041 μM, 0.110 μM, or 0.707 μM. In an embodiment, a pyrazoloquinoline compound of the invention inhibits PI3Kγ at an IC₅₀ value of 0.005-0.050 μM. An embodiment of a pyrazoloquinoline compound of the invention may inhibit PI3Kγ at an IC₅₀ value of 0.010-0.040 μM. Different embodiments of a pyrazoloquinoline compound of the invention may inhibit PI3Kγ at an IC₅₀ value of 0.015 μM, 0.026 μM, or 0.027 μM. An embodiment of a pyrazoloquinoline compound of the invention may inhibit PI3Kδ at an IC₅₀ value of 0.005-0.050 μM. An embodiment of a pyrazoloquinoline compound of the invention may inhibit PI3Kδ at an IC₅₀ value of 0.010-0.040 μM. An embodiment of a pyrazoloquinoline compound of the invention was found to inhibit PI3Kδ at an IC₅₀ value of 0.013 μM or 0.027 μM.

In an embodiment, a pyrazoloquinoline compound of the invention may inhibit MELK at an IC₅₀ value of <100 nM, or <500 nM, or <2000 nM. In another embodiment, a pyrazoloquinoline compound of the invention may inhibit MELK at an IC₅₀ value of 0.001-0.850 μM. An embodimentof a pyrazoloquinoline compound of the invention may inhibit MELK at an IC₅₀ value of 0.006-0.560 μM. In a further embodiment, a pyrazoloquinoline compound of the invention may inhibit MELK at an IC₅₀ value of 0.005-0.350 μM. An embodiment of a pyrazoloquinoline compound of the invention may inhibit PI3Kα at an IC₅₀ value of 0.005-0.100 μM. An embodiment of a pyrazoloquinoline compound of the invention may inhibit MELK at an IC₅₀ value of 0.005 μM, 0.016 μM, 0.022 μM, 0.133 μM, 0.310 μM, 0.559 μM, or 0.750 μM. Another embodiment of a pyrazoloquinoline compound of the invention may potently and selectively inhibit both PI3Kα (IC₅₀=0.008 μM) and MELK (IC₅₀=0.008 μM). In another embodiment, a pyrazoloquinoline compound of the invention may potently and selectively inhibit both PI3Kα (IC₅₀=0.010 μM) and MELK (IC₅₀=0.016 μM). In an embodiment, a pyrazoloquinoline compound of the invention may potently and selectively inhibit both PI3Kα (IC₅₀=0.046 μM) and MELK (IC₅₀=0.022 μM). An embodiment of a pyrazoloquinoline compound of the invention may potently and selectively inhibit both PI3Kα (IC₅₀=0.009 μM) and MELK (IC₅₀=0.133 μM).

In an embodiment, a pyrazoloquinoline compound of the invention may inhibit mTOR at IC₅₀ values of from 0.050-900 μM. An embodiment of the pyrazoloquinoline compounds of the invention inhibit mTOR at IC₅₀ values from 0.100-850 μM. In an embodiment, a pyrazoloquinoline compound of the invention may inhibit mTOR at an IC₅₀ value of 0.084 μM, 0.443 μM, 0.354 μM, 0.470 μM, 0.554 μM, or 0.841 μM.

An embodiment of the pyrazoloquinoline compounds of the invention may inhibit PDGFR or a mutant thereof, e.g., PDGFRα(D842V), at IC₅₀ values of from 0.001-550 μM. In another embodiment, the pyrazoloquinoline compounds of the invention may inhibit PDGFR or a mutant thereof, e.g., PDGFRα(D842V), at IC₅₀ values of from 0.005-500 μM. An embodiment of the pyrazoloquinoline compounds of the invention may inhibit PDGFR or a mutant thereof, e.g., PDGFRα(D842V), at IC₅₀ values of from 0.010-300 μM. An embodiment of the pyrazoloquinoline compounds of the invention may inhibit PDGFR or a mutant thereof, e.g., PDGFRα(D842V), at IC₅₀ values of from 0.015-170 μM. An embodiment of a pyrazoloquinoline compound of the invention may inhibit PDGFR or a mutant thereof, e.g., PDGFRα(D842V), at an IC₅₀ value of 0.015 μM, 0.023 μM, 0.159 μM, 0.170 μM, or 0.285 μM

An embodiment of the pyrazoloquinoline compounds of the invention may inhibit MNK1 and/or MNK2 or a mutant thereof, e.g., (T385D)MNK1, at IC₅₀ values of from 0.001-250 μM or by >70% at a compound concentration of 1 μM

An embodiment of the pyrazoloquinoline compounds of the invention may inhibit at medically and clinically relevant kinases, such as one or more of, but not limited to, ABL1, ABL2, ALK4, ARKS, AUR A, AXL, BLK, BMX, BRK, BTK, CAMKK2, CDK1, CDK2, CDK3, CDK5, CDK7, CK1δ, CK1ε, CK2α, CK2α2, CLK1, CLK2, CLK3, CLK4, c-MER, c-Src, DYRK1A, DYRK1B,DYRK2, DYRK3, EGFR, EGFR(L858R), EPHA7, FER, FGR, FLT3, FLT3(D835Y), FLT4, FMS, FYN, GCK, GSK3α, GSK3β, HCK, HGK, HIPK2, HIPK3, HIPK4, IRAK1, IRAK4, ITK, KDR/VEGFR2, KIT, KIT(V654A), KIT(D816V), LCK, LOK, LYN, MELK, MER, MLCK2, MLK1, MNK1, MNK2, MST1, MST2, mTOR, MUSK, NEK1, NEK3, PASK, PDGFRα, PDGFRα(V561D), PDGFRα(T674I), PDGFRα(D842V), PDGFRβ, PIM-1, PKCδ (delta), PKCμ (mu), PKCν (nu), PKD2, RET, RIPK2, ROS, RSK1, RSK2, RSK3, RSK4, SRC, STK33, TAK1, TAOK1, TAOK3, TRKA, TRKB, TRKC, TTK, TXK, TYK, TYK2, YES, ZAK, and ZAP70 kinases, or mutant, mutationally activated, or variant forms thereof, by >50% at a compound concentration of 1 μM.

The foregoing IC50 values represent averages of multiple samples (n) tested. In most cases, n typically equals 1-10.

An embodiment of the present invention encompasses a compound, in particular, a pyrazoloquinoline compound as described herein, that may exhibit potent PI3K inhibitory activity and selectivity over an unrelated target, e.g., IRK, and exhibits desirable pharmacokinetic properties and in vivo efficacy. An embodiment of a pyrazoloquinoline compound of the invention may inhibit a particular class or isoform of PI3K, e.g., PI3Kα or p110α; P13Kβ or p110β; PI3Kγ or p110γ; or PI3Kδ or p110δ, or a mutant form thereof. In another embodiment, a pyrazoloquinoline compound of the invention may not inhibit a particular class or isoform of PI3K. An embodiment of a pyrazoloquinoline compound of the invention may exhibit selective and potent inhibition of a kinase (e.g., protein kinase) that is associated with a disease, condition, or pathology, such as, but not limited to, cancers, tumors, neoplasms, malignancies, inflammatory diseases, or cardiovascular diseases. In the protein kinase profiling assay, compounds of the invention also exhibited inhibitory activity against TRKC. For example, compounds demonstrated >97% inhibition of TRKC at 1 μM, e.g., 1107, 1108, 1111, 1121; compounds demonstrated >80% inhibition of TRKC at 1 μM, e.g., 1110; compounds demonstrated ≧70% inhibition of TRKC at 1 μM, e.g., 1122, 1113; compounds demonstrated ≧75% inhibition of TRKC at 1 μM, e.g., 1116, 1112, 1125; compounds demonstrated >60% inhibition of TRKC at 1 μM, e.g., 1124, 1123, 1115; and compounds demonstrated >50% inhibition of TRKC at 1 μM, e.g., 1117. Compounds of the invention further inhibited PIM-1 as assessed in the protein kinase profiling assays. Compounds demonstrated >95% inhibition of PIM-1 at 1 μM, e.g., 1111, 1121; compounds demonstrated >85-95% inhibition of PIM-1 at 1 μM, e.g., 1107; compounds demonstrated ≧60-85% inhibition of PIM-1 at 1 μM, e.g., 1110 1108, 1125, 1115, 1117, 1116; and compounds demonstrated 50-60% inhibition of PIM-1 at 1 μM, e.g., 1123, 1115, 1113, 1122.

In an embodiment of the invention, exemplary compounds of the invention exhibited biochemical potency (e.g., inhibitory activity (IC₅₀ of less than 10 nM) against PI3K; high selectivity (inactivity) against an irrelevant target (e.g., IRK); and potency in cell-based assays. According to an embodiment, a PI3K inhibitor compound of the invention also targeted and selectively inhibited specific kinases other than PI3K. More particularly, an exemplary compound, at a concentration of 1 μM in the presence of 10 μM ATP, may demonstrate ≧90% inhibition of BLK1, CDK1, CDK2, CK2α2, CLK1, CLK4, LCK, DYRK2, FLT3, GCK, HCK, IRAK1, IRAK4, ITK, c-MER, LYN, MELK, PDGFRβ, PIM-1, TRKC; ≧80% inhibition of ABL1, BRK, CDK5, DYRK3, SRC, FLT4, FYN, HIPK3, HIPK4, MLCK, MNK1, PDGFRα, TAK1 and YES; or ≧70% inhibition of ARKS, AXL, CK2a, CLK2, CLK3, FGR, FMS, MST1, MUSK, NEK1, ROS, RSK1, TAOK and TRKB in a kinase profiling assay employing high throughput radiometric techniques and nanoliter volume technology (Reaction Biology Corporation, Malvern Pa.). Such an assay may be employed to profile the activity of the compounds of the invention against a panel of a large number of protein kinases (i.e. 254), which is understood to be representative of all kinase families and also includes clinically relevant mutants of certain oncogenic kinases.

In an embodiment of the invention, exemplary compounds of the invention exhibited biochemical potency (e.g., inhibitory activity (IC50 of less than 10 nM) against PI3K, namely, PI3Kα; high selectivity (inactivity) against an irrelevant target (e.g., IRK); and potency in cell-based assays. According to an embodiment, a PI3K inhibitor compound of the invention also targeted and selectively inhibited specific kinases other than PI3Kα. More particularly, an exemplary pyrazoloquinoline compound, at a concentration of 1 μM in the presence of 10 uM ATP, demonstrated ≧90% inhibition of PDGFRα(D842V), TRKc, FLT3(D835Y), DYRK2, FLT3, c-MER, MELK, CLK1, GSK3α/β and CK2α2; ≧80% inhibition of RIPK2, HCK, PIM-1, IRAK4 and CK2α; and ≧70% inhibition of ABL1, ITK, BMX/ETK, MLK1, c-KIT(D816V), MNK2, mTOR and BTK in a kinase profiling assay employing high throughput radiometric techniques and nanoliter volume technology (Reaction Biology Corporation, Malvern Pa.). In an embodiment, another pyrazoloquinoline PI3Kα inhibitor compound of the invention, at a concentration of 1 μM in the presence of 10 μM ATP, demonstrated ≧90% inhibition of BLK1, CDK1, CDK2, CK2α2, CLK1, CLK4, LCK, DYRK2, FLT3, GCK, HCK, IRAK1, IRAK4, ITK, c-MER, LYN, MELK, PDGFRβ, PIM-1, TRKC; ≧80% inhibition of ABL1, BRK, CDK5, DYRK3, SRC, FLT4, FYN, HIPK3, HIPK4, MLCK, MNK1, PDGFRα, TAK1 and YES; or ≧70% inhibition of ARK5, AXL, CK2a, CLK2, CLK3, FGR, FMS, MST1, MUSK, NEK1, ROS, RSK1, TAOK and TRKB in a kinase profiling assay employing high throughput radiometric techniques and nanoliter volume technology (Reaction Biology Corporation, Malvern Pa.). Such an assay may be employed to profile the activity of the compounds of the invention against a panel of a large, yet non-exhaustive, number of protein kinases (i.e. 250), which those in the art will appreciate are representative of all kinase families; the panel also includes clinically relevant mutants of certain oncogenic kinases.

Results of experiments demonstrating exemplary compounds of the invention capable of inhibiting various PI3K isoforms and mutants thereof and some protein, e.g., serine/threonine, kinases are presented below. The recorded values are measured in nM concentration at 50% inhibition. Measurements were obtained as described above for determining the biochemical activities of the compounds of the invention. Additionally, the tested compounds did not inhibit the protein kinase IRT. Several compounds of the invention that significantly inhibited PI3Kα at an IC50 level of <10 nM, e.g., 1110, 1108, 1107, 1118, 1122, 1119, and other compounds that inhibited PI3Kα at an IC50 level of >20 nM, e.g.,1078, 1119, 1114, 1111,1121 and 1120. Compounds of the invention also inhibited PI3Kα mutants, such as PI3Kα(E545K), at an IC50 level of <10 nM, e.g., 1107, or from 15-55 nM, e.g., 1078, 1110, and PI3Kα(H1047R), at an IC50 level of 5-25 nM, e.g., 1107, or >25 nM, e.g., 1078, 1110. Compounds of the invention inhibited PI3Kβ at an IC50 level of 35-50 nM, e.g., 1107, and at an IC50 level of >50 nM, e.g., 1078, 1110. Compounds of the invention inhibited PI3Kδ at an IC50 level of <15 nM, e.g., 1078, and at an IC50 level of 10-35 nM, e.g., 1078, 1107, 1110. Compounds of the invention inhibited PI3Kγ at an IC50 level of 10-25 nM, e.g.,1078, 1107, 1110. In addition, following the profiling assays conducted to determine inhibitory activity against various protein kinases as described supra, compounds of the invention were found to exhibit inhibitory activity against MELK at an IC50 value of <12 nM, e.g., 1107; at an IC50 value of 12-20 nM, e.g., 1108, 1121; at an IC50 value of 20-30 nM, e.g., 1111; and at an IC50 value of >30 nM, e.g., 1078, 1110, 1108, 1118, 1114, 1125, 1126, 1124, 1123, 1115, 1117,1112, 1116, 1113, 1122, and against PDGFRα(D842V) at an IC50 value of 20-25 nM, e.g., 1817, 1529; at an IC50 value of >100 nM, e.g., 1112, 1113, 1114, 1121, 1110, 1108; and at an IC50 value of >1 μM, e.g., 1118, 1125, 1123 and 1115.

Furthermore, compounds of the invention were assayed in cell-based assays for their ability to inhibit phospho-AKT in human cancer cells and proliferation of such cells expressing PI3K, employing the methods described hereinabove. Multiple measurements of such pyrazoloquinoline compounds of the invention were recorded as means of the concentration in nM yielding 50% inhibition of p-AKT in U-87 MG cells or IGF-1-stimulated A549 cells. The inhibition of phosphorylation of the S473 site in AKT (p-AKT(S473)) was evaluated. For example, the potency of AKT phosphorylation inhibition of a compound of the invention, e.g., 1078, as determined by measured IC₅₀ values, was found to be 88 nM against the p-AKT(S473) phosphorylation site in U-87 MG cells, and 43 nM against the p-AKT(S473) phosphorylation site in A549 cells. As another example, compound 1107 demonstrated an IC₅₀ value of 54 nM against the p-AKT(S473) phosphorylation site in U-87 MG cells, and 189 nM against the p-AKT(S473) phosphorylation site in A549 cells, while compound 1110 demonstrated an IC₅₀ value of 81 nM against the p-AKT(S473) phosphorylation site in U-87 MG cells, and 210 nM against the p-AKT(S473) phosphorylation site in A549 cells. In addition, in preliminary experiments, exemplary compounds, 1078, 1107, and 1110, achieved 50% inhibition of U-87 MG cell proliferation at mean concentrations of 295, 470 and ND, respectively. The measurements were obtained by methods described above for determining activity of PI3K compounds of the invention.

### EXAMPLE C

### Pyrazoloquinoline Compounds of the Invention are Potent Inhibitors of Class I and Class IV PI3Ks

Compounds were assayed against the Class I PI3Kα, PI3Kβ, PI3Kγ, PI3Kδ, PI3Kα(H1047R) and PI3Kα(E545K) in the KinaseGLO assay as described in Example 2, and against the Class IV PI3K mTOR in the radiometric assay. IC₅₀ values (μM) were determined using 8-10 point half-log dose dilutions and calculated via nonlinear regression analysis using Graphpad Prism data analysis software. The IC₅₀ values of three pyrazoloquinoline compounds of the invention revealed that such compounds had high levels of potency in inhibiting PI3Ks of classes I and IV. For example, for PI3Kα, IC₅₀ values were 0.0048, 0.0092 and 0.0446; for PI3Kβ, IC₅₀ values were 0.0413, 0.1096 and 0.7070; for PI3Kδ, IC₅₀ values were 0.0084, 0.0197 and 0.0239; for PI3Kγ, IC₅₀ values were 0.0150, 0.0173 and 0.0268; for mTOR, IC₅₀ values were 0.1338, 0.4425 and 0.5338; for PI3Kα(E545K), IC₅₀ values were 0.0091, 0.0308 and 0.0377; and for PI3Kα(H1047R), IC₅₀ values were 0.0161, 0.0335 and 0.0575.

### EXAMPLE D

### Pyrazoloquinoline Compounds of the Invention are Active Against Multiple Protein Kinases

Compounds of the invention were profiled against over 250 protein kinases at a final compound concentration of 1 μM. PI3K inhibitor compounds of the invention showed distinct profiles of inhibitory activity against target kinases. For example, a parazoloquinoline compound of the invention inhibited the following kinases by 50% or greater: ABL1, ABL2, AURORA A (AUR A), AXL, BMX, BTK, CAMKK2, CDK1, CK1δ, CK1ε, CK2α, CK2α2, CLK1, CLK3, DYRK2, EPHA7, FGR, FLT3, FLT4, FYN, GSK3α, GSK3β, HGK/MAP4K4, HIPK2, IRAK1, IRAK4, ITK, KDR/VEGFR2, LOK/STK10, LYN, MELK, MER, MLK1, MNK2, mTOR, MUSK/FRAP, MUSK, PDGFRα, PIM-1, RET, RIPK2, ROS/ROS1, SRC, STK33, TRKA/NTRK1, TRKB/NTRK2, TRKC/NTRK3, TYK2, YES, ZAK/MLTK, and ZAP70. Mutated kinases inhibited: EGFR(L858R), FLT3(D835Y), KIT(V654A), PDGFRα(V561D), PDGFRα(T674I) and PDGFRα(D842V). Another pyrazoloquinoline compound of the invention inhibited the following kinases by 50% or greater: ABL1, ABL2, ALK4, AXL, BLK, BRK, CAMKK2, CDK1, CDK2, CDK3, CDK5, CDK7, CK1δ, CK1ε, CK2α, CK2α2, CLK1, CLK2, CLK3, CLK4, DYRK1/DYRK1A, DYRK2, DYRK3, EPHA7, FER, FGR, FLT3(CD), FLT4NEGFR3, FMS, FYN, GCK/MAP4K2, GSK3α, HCK, HGK/MAP4K4, HIPK2, HIPK3, HIPK4, IRAK1, IRAK4, ITK, KDRNEGFR2, LCK, LOK/STK10, LYN, MELK, MER, MLCK2/MYLK2, MLK1/MAP3K9, MNK1, MST1/STK4, MST2/STK3, MUSK, NEK1, PDGFRα, PDGFRβ, PIM-1, PKCδ(delta), PKCμ(mu)/PKD1, PKCν(nu)/PKD3, PKD2/PRKD2, RET, RIPK2, ROS/ROS1, RSK1, RSK2, RSK3, RSK4/RPS6KA6, TAK1/MAPK3K7, TAOK1, TAOK3/JIK1, TRKA/NTRK1, TRKB/NTRK2, TRKC/NTRK3, TTK, TXK, YES and ZAP70.

### EXAMPLE E

### Biochemical Potency of Pyrazoloquinoline Compounds of the Invention Against Select Protein Kinases

Compounds of the invention were assayed against the select kinases using the KinaseGLO assay (MELK) or the radiometric assay (PIM, TRKC, PDGFRα(D842V), MNK1 and mTOR). IC₅₀ values were determined using 8-10 point half-log dose dilutions and calculated via nonlinear regression analysis using Graphpad Prism data analysis software. The Example 6 Table below shows that four representative pyrazoloquinoline compounds of the invention, P, Q, R, T, have biochemical activity against the tested protein kinases.

### EXAMPLE F

### Cellular Activity of the Pyrazolquinoline PI3K Multiplex Kinase Inhibitors of the Invention

Experiments were conducted to assess the ability of pyrazolquinoline PI3K multiplex kinase inhibitors of the invention to block AKT phosphorylation in growth factor stimulated A549 NSCLC cells and U-87 MG GBM cells. A549 and U-87 MG cells were incubated with 5 different pyrazoloquinoline compounds of the invention, P-T, for one hour and the levels of phosphorylated AKT (serine 473 and/or threonine 308), as a measure of PI3K activity, were detected and quantified using an in-cell western blot assay.

### EXAMPLE G

### Inhibition of Human Tumor Cell Proliferation by Pyrazolquinoline PI3K Multiplex Kinase Inhibitors of the Invention

Experiments were conducted to examine the ability of pyrazoloquinoline PI3K multiplex kinase compounds of the invention to inhibit the proliferation of a variety of human tumor cells. Human tumor cell lines of multiple histotypes were incubated with four different pyrazoloquinoline compounds of the invention, for 72 hours as described. After 72 hours, cell proliferation was measured and quantified using the Cell Titer-GLO assay. Cell proliferation EC₅₀ values (μM values) were calculated via non-linear regression analysis using GraphPad Prism data analysis software. Cell line histotypes: (GBM) glioblastoma; (MB) medulloblastoma; (CRC) colorectal carcinoma; (BC) breast carcinoma; (PaC) pancreatic carcinoma; (NSCLC) non-small cell lung carcinoma.

### EXAMPLE H

### Inhibition of Human Tumor Cell Proliferation by Pyrazolquinoline PI3K Multiplex Kinase Inhibitors of the Invention

Experiments were carried out to assess the ability of compounds of the invention to inhibit the proliferation of endothelial cells using the endothelial cell line HUVEC and HMVEC. HUVEC and HMVEC endothelial cells were dosed with using half-log, eight-point compound dilutions ranging from 0.001 μM-10 μM concentrations of control endothelial cell inhibitor compounds and a parazoloquinoline compound of the invention. Cells were then stimulated with VEGF₁₋₁₆₅ for 24 hours, and cell proliferation (as new DNA synthesis) was detected and quantified using the Cell Proliferation (BrdU) assay as described. Cell proliferation EC₅₀ values were calculated via non-linear regression analysis using GraphPad Prism data analysis software. A compound of the invention showed potent inhibition of endothelial cell proliferation, i.e., an EC₅₀ of 0.0505 against HUVECs and ., an EC₅₀ of 0.0314 against HMVECs.

### EXAMPLE I

### Pyrazoloquinoline PI3K Multiplex Kinase Inhibitor Compounds of the Invention Induce Human Tumor Cell Death Through the Activation of Caspases

Compounds of the invention which function as PI3K multiplex kinase inhibitors induce human tumor cell death through the activation of caspases. HCT116 cells were treated overnight with a pyrazoloquinoline compound of the invention, or with a commercially available dual PI3K(α,β,δ,γ)/mTOR inhibitor, or with a commercially available PI3K(α,β,δ,γ)-only inhibitor at concentrations of 0.5, 1.0 and 5.0 μM. After treatment, cell lysates were prepared and analyzed for the presence of active caspases (cleaved caspase 3) and cleaved PARP. Only cells treated with a compound of the invention showed both cleaved PARP and cleaved caspase 3 at a compound concentration of 5.0 μM. In addition, a PI3K multiplex kinase inhibitor compound of the invention induced caspase activity in human tumor HCT116 cells that had been incubated with the compound for 24 hours prior to detection of caspase activity via CaspaseGLO assay described in Example 2. The EC₅₀ values of caspase activity were calculated using non-linear regression analysis (Graphpad Prism), with staurosporine control determining the assay upper and lower limits. The EC₅₀ value (μM) for staurosporine was 0.007, while the EC₅₀ value (μM) for the compound of the invention was 0.396.

### EXAMPLE J

### Pyrazoloquinoline PI3K Multiplex Kinase Inhibitor Compounds of the Invention Block eIF-4E Phosphorylation

To assess whether PI3K multiplex kinase inhibitor compounds of the invention blocked eIF-4E phosphorylation, HCT116 cells were incubated for 24 hours with either 0, 0.5, 1.0, or 5.0 μM of a pyrazoloquinoline compound of the invention. Cell lysates were prepared and analyzed by western blot for changes in eIF-4E phosphorylation, which indicates MNK1/2 inhibition. At compound concentrations of 1.0 and 5.0 μM, the phosphorylation of p-(S209)eIF-4E was clearly reduced relative to 0 and 0.5 μM concentrations. By contrast, no inhibition of phosphorylation of p-(T202/S204)ERK1/2 was observed at any concentration of the compound of the invention.

### EXAMPLE K

### Pyrazoloquinoline PI3K Multiplex Kinase Inhibitor Compounds of the Invention Induce Cell Death in Human Tumor Cell Lines Including Cell Lines Expressing Mutated Ras

Compounds of the invention were assayed for their ability to induce cell death in human tumor cell lines expressing mutated Ras. The ability of a PI3K inhibitor compound of the invention to inhibit tumor cell growth (cytostatic) or induce tumor cell death (cytotoxic) was evaluated in a panel of human tumor cell lines incubated with increasing concentrations of compounds for 72 hours. The mutational status of the tumor cell lines was obtained from the Wellcome Trust Sanger Institute, CGP Cancer Cell Line Project, Cambridge, UK. The relative values for cytostatic and cytotoxic activities are scored as follows: (i) +++<0.1 μM; (ii) ++<0.5 μM; (iii) +<1.0 μM; (iv) − no significant activity.

### Synthesis

Compounds of the invention can be prepared by the following examples.

### EXAMPLES

### Example 1

### Preparation of PI3K Inhibitor Compounds

General procedures to prepare the title compounds, abbreviations, and five sub-sections which describe the preparation of building blocks, intermediates, and final compounds of the invention are provided hereinbelow.

I. Preparation of building blocks

II. Preparation of scaffold H (variation on 8-position of pyrazolo[3,4-c]quinoline),

III. Preparation of scaffold J=(variation on 3-position of pyrazolo[3,4-c]quinoline),

IV. Preparation of scaffold R (variation on 1-position of pyrazolo[3,4-c]quinoline),

V. Preparation of other scaffolds (replacement of pyrazolo moiety).

General Procedures

Solvents were dried by distillation from a drying agent: THF from Na/benzophenone; CH₂Cl₂ from CaH₂. ¹H NMR was recorded on a 400 MHz (Avance III) spectrometer and the chemical shifts are reported downfield from TMS as an internal standard, and for peak assignmet, s (sigle), b (broad), d (double), t (triple), m (multiple), J=(Coupling constant, Hz). LC-MS was recorded on Agilent LC/MSD 1200 (mobile phase: CH₃CN+10 mM NH₄HCO₃ with H₂O, gradient from 5% to 95%. HPLC was recorded on Agilent LC/MSD 1200 (Mobile phase: H₂O/CH₃CN/10 mMNH₄HCO₃,). Chromatogram was visualized with UV light (254 and 214 nm). Flash Chromatography was performed on silica gel (200-400 mesh).

Abbreviations

Ac: acetyl

Bn: benzyl

BOC: t-butoxycarbonyl

Bz: benzoyl

Cbz: benzyloxycarbonyl

MOM: methoxymethyl

Ms methanasulfonyl

piv: pivalic

TMS: trimethylsilyl

Ts: toluenesulfonyl

DIBAL-H: diisobutylaluminum hydride

n-BuLi: n-Buthyl Lithium

DIPEA: diisopropylethylamine

DMAP: 4-dimethylamino pyridine

DMF: dimethylformamide

DMCO: acetone

DMSO: dimethylsulfoxide

mCPBA: meta-Chloroperoxybenzoic acid

NBS: N-bromosuccunimide

Pd₂(dba)₃: tris(dibenzylideneacetone)dipalladium

TMEDA: tetramethylethylenediamine

DCM: dichloromethane

EA: ethyl acetate

PE: petroleum ether

TEA: triethyl amine

TFA: trifluroacetic acid

THF: terahydrofuran

I. Preparation of Building Blocks

Building blocks used in syntheses of final compounds are provided in sub-sections (I-1) halides, (I-2) boronic acids and (I-3) boronate esters below.

I-1. Preparation of Halides

The invention provides new halide compounds. Building blocks (bromides and iodides) for such compounds were prepared by following nine generic methods (i.e., Methods A, B, C, D, E, F, G, H, and I).

Method A

Replace halide with amine or alkoxy group.

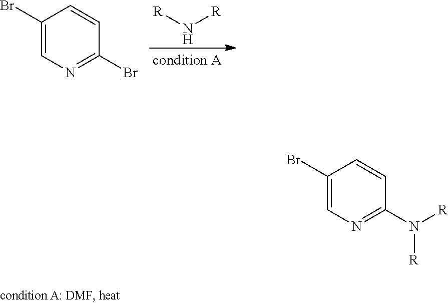

### Example for 4-(5-bromopyridin-2-yl)morpholine

A solution of 2,5-dibromopyridine (1.5 g, 6.3 mmol), L-Proline (0.08 g, 1.3 mmol), pyrrolidine (0.9 g, 0.0125 mol), AcOK (3.2 g, 33 mmol) and Cu I (0.3 g, 1.3 mmol) in DMF (40 mL) was stirred under N₂ and at 85° C. for 12 h. The solution was filtered to remove the catalyst. The residue was diluted with H₂O and was extracted with EA. The organic layer was washed with brine, dried over Na₂SO₄ and filtered. The filtrate was evaporated to give the target product (0.9 g, 63%). MS (m/z) (M⁺+H): 243, 245.

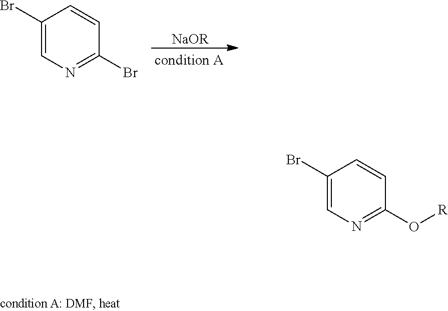

### Example for 5-bromo-2-ethoxypyridine

Freshly cut sodium (74 mg, 3.2 mmol) was dissolved in ethanol (1.2 ml) followed by addition of 2,5-dibromopyridine (236 mg, 1 mmol) in DMF (3 ml). The mixture was stirred for 1 h at 80° C., and cooled to rt. The reaction was quenched with water (4 mL) and extracted with ether three times. The combined organic layer was washed with brine, dried over Na₂SO₄, and concentrated in vacuo. The residue was purified by flash column chromatography to afford the target product as a white solid (192 mg, 95%). MS (m/z) (M⁺+H): 202, 204.

Method B

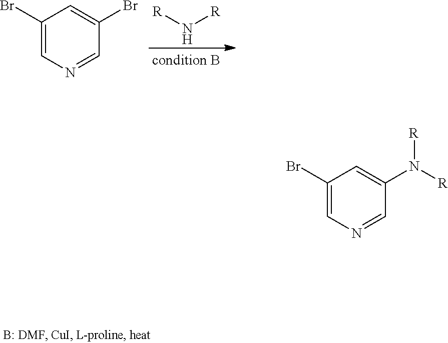

### Example for 3-bromo-5-(1H-imidazol-1-yl)pyridine

A solution of 3,5-dibromopyridine (3 g, 12.7 mmol), L-Proline (0.15 g, 1.3 mmol), imidazole (1.73 g, 25.4 mmol), AcOK (6.5 g, 66.3 mmol) and CuI (0.49 g, 2.6 mmol) in DMF (40 mL) was stirred under N₂ and at 85° C. for 12 h. The solution was filtered to remove the catalyst. The residue remaining was diluted with H₂O and was extracted with EA and the organic layer was washed with brine, dried over Na₂SO₄ to give 3-bromo-5-(1H-imidazol-1-yl)pyridine (1.3 g, 46%). MS (m/z) (M⁺+H): 224 and 226.

### Example for 5-bromo-N-isopropylpyridin-3-amine

A solution of 3,5-dibromopyridine (3 g, 12.7 mmol), L-Proline (0.15 g, 1.3 mmol), isopropylamine (1.47 g, 25 mmol), AcOK (6.5 g, 66.3 mmol) and CuI (0.49 g, 2.6 mmol) in DMF (40 mL) was stirred under N₂ and at 85° C. for 12 h. The solution was filtered to remove the catalyst. The remaining residue was diluted with H₂O and was extracted with EA, and the organic layer was washed with brine, dried over Na₂SO₄, and evaporated to give 5-bromo-N-isopropylpyridin-3-amine (1.39 g, 51%). MS (m/z) (M⁺+H): 215, 217.

Method C

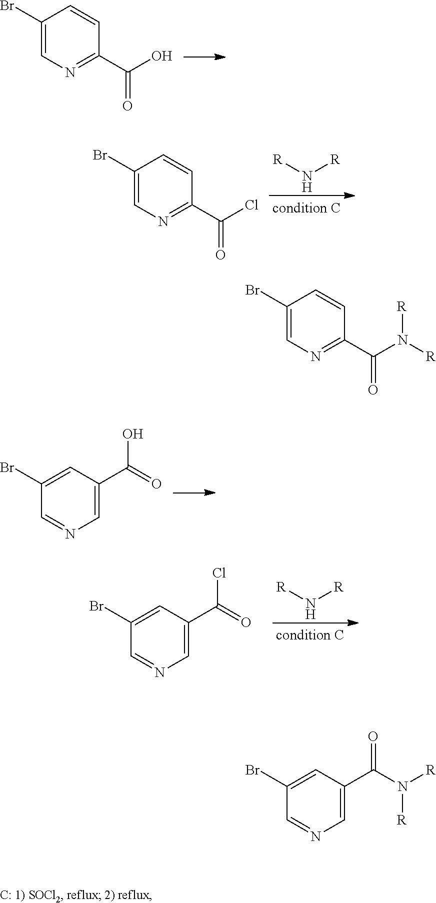

### Example for N-benzyl-5-bromonicotinamide

A solution of 5-bromonicotinic acid (10 g, 49.5 mmol) in SOCl₂ (80 mL) was stirred at reflux temperature for 12 hours. SOCl₂was evaporated in vacuum to give 5-bromonicotinoyl chloride (10.5 g, 95%). A solution of 5-bromonicotinoyl chloride (2.06 g, 9.4 mmol) and Et₃N (15 mL) was stirred in DCM (80 mL), then Benzylamine(1 g, 9.4 mmol) was added into the above solution at 0° C., which was stirred at room temperature for 12 h. The solution was diluted with H₂O, and extracted with DCM. The organic layer was washed with brine, dried over Na₂SO₄, and filtered. The filtrate was evaporated to give N-benzyl-5-bromonicotinamide (2.98 g, 86.5%). MS (m/z) (M⁺+H): 291 and 293.

Method D

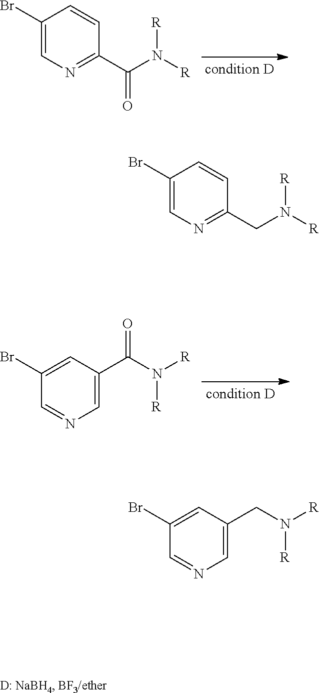

### Example for 4-((5-bromopyridin-3-yl)methyl)morpholine

A solution of (5-bromopyridin-3-yl)(morpholino)methanone (2.9 g, 0.1 mmol) and NaBH₄ (5 g, 13.3 mmol) was stirred in THF (80 mL) for 1 h, then BF₃.C₂H_{S}OC₂H₅ (10 mL) was added into the above solution at 0° C. The mixture was stirred at room temperature for 3 d. The reaction mixture was then filtered to remove solids. The filtrate was evaporated in vacuum and purification (EA:PE=1:1) to give 4-((5-bromopyridin-3-yl)methyl)morpholine (2.05 g, 80%). MS (m/z) (M⁺+H): 257 and 259.

Method E

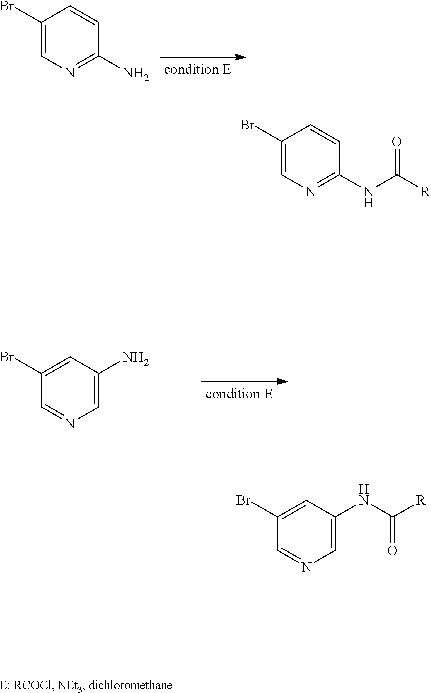

### Example for N-(5-bromopyridin-2-yl)benzamide

A solution of 5-bromopyridin-2-amine (2 g, 11.5 mmol) and Et₃N (15 mL) was stirred in DCM (80 mL), then BzCl (3.2 g, 23 mmol) was added into the above solution at 0° C., which was stirred at room temperature for 12 h. The solution was diluted with H₂O and was extracted with DCM. The organic layer was washed with brine, dried over Na₂SO₄ to give N-(5-bromopyridin-2-yl)benzamide (2.8 g, 90%). MS for C₁₂H₉BrN₂O (m/z) (M⁺+H): 277, 279.

Method F

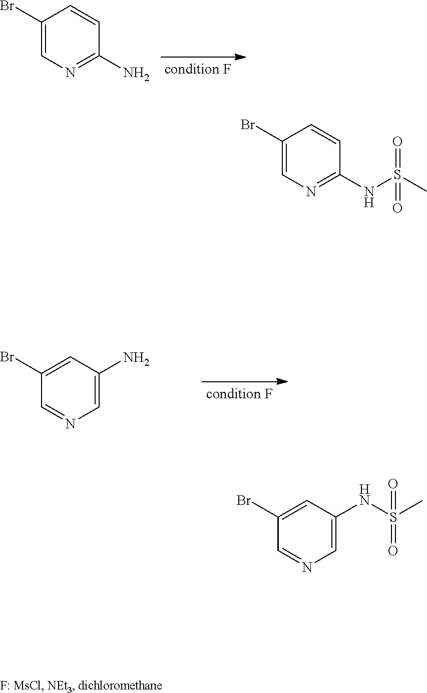

### Example for N-(5-bromopyridin-3-yl)methanesulfonamide

A solution of 5-bromopyridin-3-amine (1.73 g, 10 mmol) and pyridine (15 mL) was stirred in DCM (80 mL), then MsCl (1.15 g, 10 mmol) was added into the above solution at 0° C., which was stirred at room temperature for 12 h. The solution was diluted with H₂O and was extracted with DCM. The organic layer was washed with brine, dried over Na₂SO₄ to give N-(5-bromopyridin-3-yl)methanesulfonamide (2.8 g, 90%). MS for C₁₂H₉BrN₂O (m/z) (M⁺+H): 277 279.

Method G

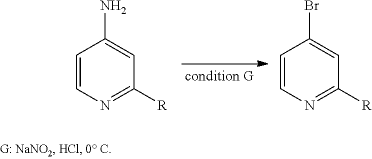

### Example for 1-(4-bromopyridin-2-yl)piperazine

A solution of 1-(4-aminopyridin-2-yl)piperazine (6.9 g, 39 mmol) in 48% HBr (300 mL) was stirred and cooled to 0° C. Then a solution of NaNO₂ (2.5 g, 39 mmol) in 15 mL water was added dropswise with controlling the inner temperature under 0° C. The reaction mixture was stirred for 20 mins and CuBr (5.38 g, 39 mmol) was added, and then warned to room temperature for 3 h. The mixture was basified with KOH to PH=10, and extracted with ethyl acetate (500 mL×3). The combined ethyl acetate extracts was dried over Mg₂SO₄, filtered, concentrated give crude, which was purified via column chromatography (PE:EA:CH3OH=1:1:0.1) to afford (1-(4-bromopyridin-2-yl)piperazine) (2.26 g, 24% for 2 steps), >90% pure. MS (m/z) (M⁺+H): 242, 244.

Method H, Multistep Reactions for Hetero Aryls

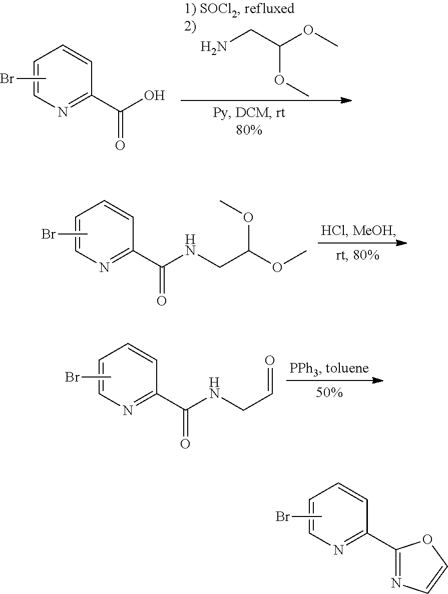

### Example for 5-bromo-2-(oxazol-2-yl)pyridine

5-bromopicolinic acid (0.5 g, 2.5 mmol) was dissolved in SOCl₂ (2 mL) and refluxed for 2 h, then SOCl₂ was removed under vacuum to give compound 5-bromopicolinoyl chloride (0.50 g, 95%).

To a solution of 5-bromopicolinoyl chloride (0.5 g, 2.5 mmol) in EtOH (30 mL) was added 2,2-dimethoxyethanamin (0.26 g, 2.5 mmol) and TEA (1.1 g, 10 mml). The reaction mixture was refluxed for 2 h, concentrated and chromatography (EA:PE=1:5) to give compound 5 (0.58 g, 80%) as a light yellow solid.

To a solution of 5-bromo-N-(2,2-dimethoxyethyl)picolinamide (0.58 g, 2.0 mmol) in DCM (30 mL) was added concentrated HCl (2 mL). The reaction mixture was stirred for 4 h at rt, saturated NaHCO₃ solution was added to PH 7.5, organic layer was washed with brine, dried over MgSO₄, concentrated to give 5-bromo-N-(2-oxoethyl)picolinamide (0.37 g, 80%) as white solid. To a solution of 5-bromo-N-(2-oxoethyl)picolinamide (0.24 g, 1 mmol) in Toluene (10 mL) was added Ph₃PO (0.56 g, 2 mmol). The reaction mixture was refluxed overnight, concentrated and chromatography (EA:PE=1:5) to give 5-bromo-2-(oxazol-2-yl)pyridine (0.098 g, 50%) as white solid. MS (m/z) (M⁺+H): 225, 227.

Method I, Other Hetero-Aryl Building Blocks

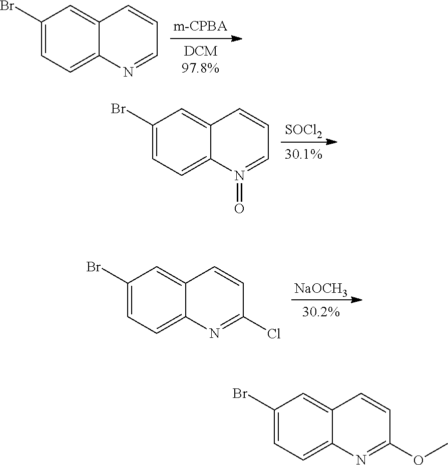

To a solution of 6-bromoquinoline (17.3 g, 83.17 mmol) in DCM (180 ml) was added m-CPBA (21.53 g,124.8 mmol) in portions at 0° C. The mixture was stirred at R.T. for 3 h. The mixture was quenched with saturation NaHCO₃, extracted with DCM. The organic phases were combined and dried over Na₂SO₄, and filtered. The filtrate was evaporated in vacuo to give 6-bromoquinoline N-oxide (17.9 g, 97.8%).

A solution of 6-bromoquinoline N-oxide (15.4 g, 68.75 mmol) in SOCl₂ (100 ml) was heated to refluxed for 6 h and was cooled to R.T. SOCl₂ was removed in vacuo, to the residue was added water (100 ml) and DCM (200 ml). The organic layer was separated, washed with brine, dried over Na₂SO₄, and filtered. The filtrate was evaporated to give 2-chloro-6-bromoquinoline (7.8 g, 30.1%).

To a solution of Na (0.14 g, 6.0 mmol) in methanol (15 ml) was added 2-chloro-6-bromoquinoline (1.2 g, 5.0 mmol). The mixture was heated to reflux for 17 h. The mixture was cooled to R.T. and evaporated to dryness. To the residue was added water (15 ml) and DCM (20 ml). The organic layer was separated, washed with brine, dried over Na₂SO₄, and filtered. The filtrate was evaporated in vacuo to give crude product, which was purified via column chromatography (PE:EA=40:1) to yield 2-methoxy-6-bromoquinoline as off white powder (0.36 g, 30.2%). MS (m/z) (M⁺+H): 238, 240.

I-2. Preparation of Boronic Acids

The invention provides boronic acids and boronate compounds. Building blocks for such compounds were prepared by procedures similar to those which have been reported in the art.

Method 1

Method 1 is to prepare aryl boronic acids from the corresponding aryl halides under four different reaction conditions (a. b. c. and d).

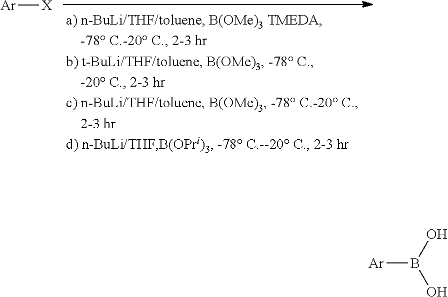

### Condition a: Example for 3-(phenylamino)phenylboronic acid)

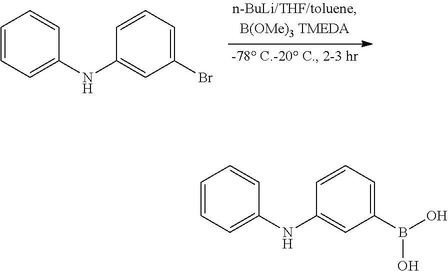

To the solution of N-(3-bromophenyl)benzenamine (2.5 g, 10 mmol), trimethyl borate (1.25g, 11 mmol) in toluene and THF (20 mL,VN=4:1) was added TMEDA (1.mL), then n-BuLi (9 mL, 22 mmol, 2.5 M in hexane) at −78° C. The reaction mixture was stirred for 1 h at −78° C., then the reaction mixture was warmed to −15° C. and quenched with 2 N HCl (5 mL, 10 mmol). The mixture was concentrated and purification by chromatography (MeOH:DCM=1:10) to give 3-(phenylamino)phenylboronic acid (0.5 g, 25%) as light yellow solid. MS (m/z) (M⁺+H): 214.

### Condition b: Example for [1,2,4]triazolo[1,5-a]pyridin-6-ylboronic acid)

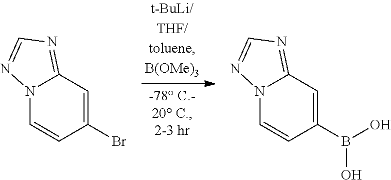

To the solution of compound 6-bromo-[1,2,4]triazolo[1,5-a]pyridine (0.2 g, 1 mmol), trimethyl borate (0.12 g, 1.2 mmol) in toluene and THF (50 mL, V/V=4:1) was added t-BuLi (1.2 mL, 1.2 mmol, 1.0 M in hexane) at −78° C. The reaction mixture was stirred for 1 h at −78° C., then the reaction mixture was warmed to −15° C. and quenched with 2 N HCl (1 mL, 2 mol). The mixture was concentrated. Purification by chromatography (MeOH:DCM=1:10) afforded [1,2,4]triazolo[1,5-a]pyridin-6-ylboronic acid (0.10 g, 55%) as light yellow solid. MS (m/z) (M⁺+H): 164.

### Condition c: Example for pyrimidin-5-ylboronic acid)

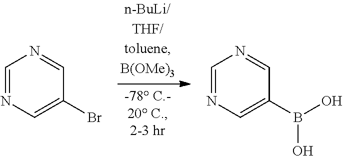

To a solution of compound 5-bromopyrimidine (8 g, 50 mmol), trimethyl borate (4.6g, 60 mmol) in toluene and THF (150 mL, V/V=4:1) was added n-BuLi (24 mL, 60 mmol, 2.5 M in hexane) at −78° C. The reaction mixture was stirred for 1 h at −78° C., then warmed to −15° C. and quenched with 2 N HCl (50 mL, 100 mmol). The mixture was concentrated and purification by chromatography (MeOH:DCM=1:10) to give pyrimidin-5-ylboronic acid (4 g, 64%) as light yellow solid. MS (m/z) (M⁺+H): 125.

### Condition d: Example for quinolin-3-ylboronic acid)

To a solution of compound 3-bromoquinoline (2.08 g, 10 mmol), triisopropyl borate (2.3g, 12 mmol) in THF (20 mL) was added n-BuLi (4 mL, 2.5 M in hexane) at −78° C. The reaction mixture was stirred for 1 h at −78° C., then warmed to −20° C. and quenched with 2 N HCl (50 mL, 100 mmol). The mixture was concentrated and purification by chromatography (MeOH:DCM=1:10) to give quinolin-3-ylboronic acid (0.97g, 56%) as light yellow solid. MS (M/Z) M⁺+H): M⁺+H=174.

Method 2

Method 2 is to prepare aryl boronic esters from the corresponding aryl halides, then further converter aryl boronic esters to the corresponding aryl boronic acids.

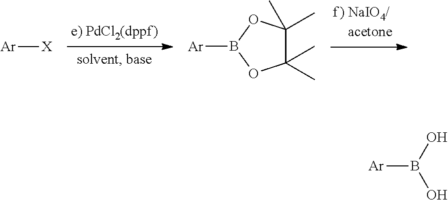

### Condition e: Example for 1-phenyl-4-(4,4,5,5-tetramethyl-1,3,2-dioxaborolan-2-yl)-1H-pyrazole

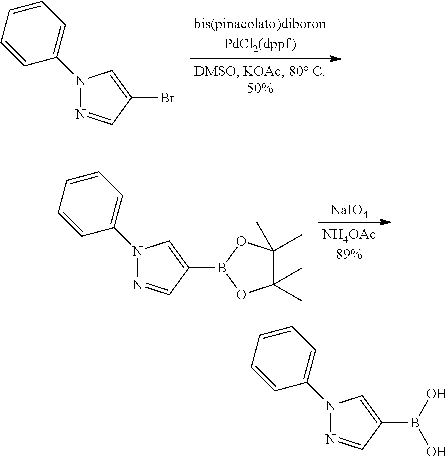

To a solution of compound 4-bromo-1-phenyl-1H-pyrazole (0.5 g, 2.3 mmol) in DMSO (50 mL) was added KOAc (0.66 g, 6.8 mmol), bis(pinacolato)diboron (0.63 g, 2.5 mmol) and PdCl₂(dppf) (0.076 g, 0.11 mmol) at room temperature. The reaction mixture was stirred overnight at 80° C. The result mixture was diluted with EA, washed with brine, dried over Na₂SO₄, concentrated, purified by chromatography (EA:PE=1:12) to give 1-phenyl-4-(4,4,5,5-tetramethyl-1,3,2-dioxaborolan-2-yl)-1H- pyrazole (0.3 g, 50%) as light yellow solid.

### Condition f: Example for 1H-pyrazol-4-ylboronic acid

To the solution of 4-pinacolatoboron-1H-pyrazole (0.6 g, 3.1 mmol) in acetone/water (30 mL, v/v=1:1) was added NaIO₄ (2.0 g, 9.3 mmol) and NH₄OAc (0.54 g, 7.1 mmol). The reaction mixture was stirred overnight at room temperature. The result mixture was concentrated to give residues, purification by chromatography (MeOH:DCM=1:5) to give compound 1H-pyrazol-4-ylboronic acid (0.3 g, 89%) as light yellow solid.

MS (m/z) (M⁺+H): 189.

I-3. Preparation of Boronate Esters

Method 1

Method 1 is to prepare the aryl boronic ester from the corresponding aryl halides with four different work-up procedures (a, b, c, and d).

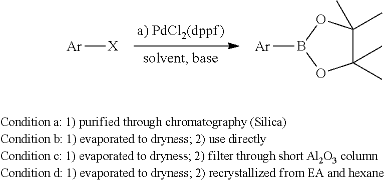

### Condition a: Example for 7-(4,4,5,5-tetramethyl-1,3,2-dioxaborolan-2-yl)quinoline

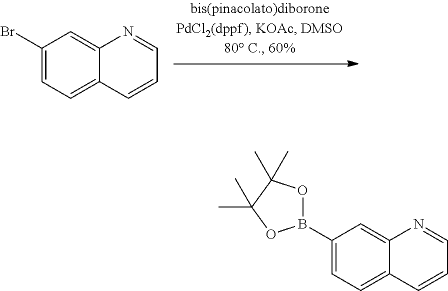

To a solution of 7-bromoquinoline (520 mg, 2.5 mmol) in DMSO (50 mL) was added KOAc (750 mg, 7.5 mmol), bis(pinacolato)diboron (690 mg, 2.75 mmol) and PdCl₂(dppf) (85 mg, 0.125 mmol) at room temperature. The reaction mixture was stirred overnight at 80° C. under N₂ protection. The resulted mixture was diluted with EA and washed with brine, dried over Na₂SO₄, and concentrated. The residue was purified by silica gel column chromatography (EA:PE=1:5) to afford 7-(4,4,5,5-tetramethyl-1,3,2 -dioxaborolan-2-yl)quinoline (380 mg, 60%) as yellow solid. MS (m/z) (M⁺+H): 255 (ester) and 173 (acid).

### Condition b: Example for 4-(5-(4,4,5,5-tetramethyl-1,3,2-dioxaborolan-2-yl)pyridin-2-yl)morpholine

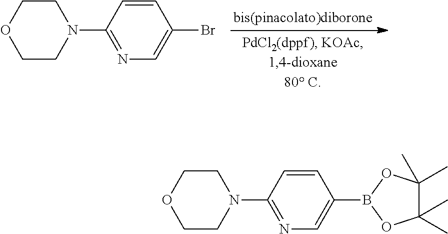

To a 25 mL round-bottom flask charged with 4-(5-bromopyridin-2-yl)morpholine (50 mg 1 eq), bis(pinacolato)diboron (1.1 eq), PdCl₂(dppf) (0.05 eq), AcOK (3 eq) in 15 mL of dioxane. The mixture was thoroughly degassed by alternately connecting the flask to vacuum and nitrogen. This solution was then heated at 85° C. for 8 h. The solvent was removed to yield a residue containing 4-(5-(4,4,5,5-tetramethyl-1,3,2-dioxaborolan-2-yl)pyridin-2-yl)morpholine, which could be used in sequential Suzuki-Miyaura Cross Coupling. MS (m/z) (M⁺+H): 291 (ester) and 209 (acid).

### Condition c: Example for 4-((5-(4,4,5,5-tetramethyl-1,3,2-dioxaborolan-2-yl)pyridin-3-yl)methyl)morpholine

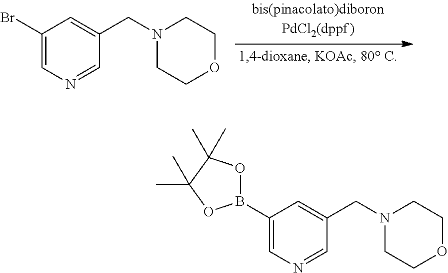

To a 25 mL round-bottom flask charged with 4-((5-bromopyridin-3-yl)methyl)morpholine (50 mg, 0.19 mmol, 1 eq), bis(pinacolato)diboron (1.1 eq), PdCl₂(dppf) (0.05 eq), AcOK (3 eq) in 15 mL of dioxane. The mixture was thoroughly degassed by alternately connecting the flask to vacuum and nitrogen. This solution was then heated at 85° C. for 8 h. The solvent was removed in vacauo. The residue was dissolved in dichloromethane and filtered through a short alumina column to remove some salt. The solution from chromatography was evaporated to yield a residue containing 4-((5-(4,4,5,5-tetramethyl-1,3,2- dioxaborolan -2-yl)pyridine -3-yl)methyl)morpholine, which could be used in sequential Suzuki-Miyaura Cross Coupling reaction. MS (m/z) (M⁺+H): 305 (ester) and 223 (acid).

### Condition d: Example for N-(5-(4,4,5,5-tetramethyl-1,3,2-dioxaborolan-2-yl) pyridin-3-yl)methanesulfonamide

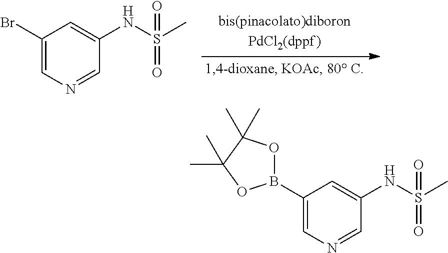

To a 100 mL round-bottom flask charged with N-(5-bromopyridin-3-yl)methane-sulfonamide (500 mg, 2 mmol, 1 eq), bis(pinacolato)diboron (1.1 eq), PdCl₂(dppf) (0.05 eq), AcOK (3 eq) in 50 mL of dioxane. The mixture was thoroughly degassed by alternately connecting the flask to vacuum and nitrogen. The solution was then heated at 85° C. for 8 h. The solvent was removed in vacauo. The residue was dissolved in ethyl acetate and filtered to remove salt. To the filtrate was added hexane to precipitate N-(5-(4,4,5,5-tetramethyl-1,3,2-dioxaborolan-2-yl)pyridin-3-yl)methane sulfonamide as a brownish yellow solid, which could be used in sequential Suzuki-Miyaura Cross Coupling reaction. MS (m/z) (M⁺+H): 299 (ester) and 217 (acid).

Method 2

Method 2 is to prepare the aryl boronic ester by alkylation, acylation, or sulfonation of the functional moieties, such as hetero ring nitrogen.

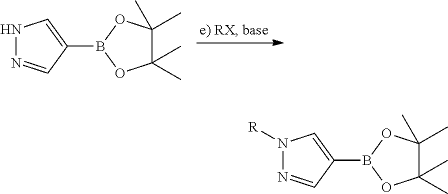

### Example for 1-(methylsulfonyl)-4-(4,4,5,5-tetramethyl-1,3,2-dioxaborolan-2-yl)-1H-pyrazole

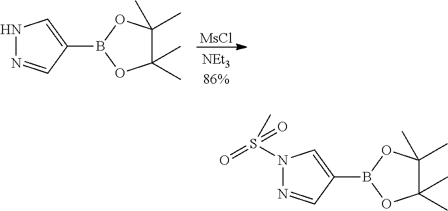

To a solution of 4-pinacolatoboron-1H-pyrazole (0.6 g, 3.1 mmol) in DCM (30 mL) was added TEA (1.0 g, 10 mmol) and MsCl (0.52 g, 4.5 mmol) at 0° C. The reaction mixture was stirred overnight at room temperature. The reaction was quenched with MeOH (1 mL), concentrated to give residues. Purification by chromatography (EA:PE=1:10) gave 1-(methylsulfonyl)-4-(4,4,5,5-tetramethyl-1,3,2-dioxaborolan-2-yl)-1H-pyrazole (0.75 g, 86%) as light yellow solid. MS (m/z) (M⁺+H): 273.

### Example for 1-methyl-4-(4,4,5,5-tetramethyl-1,3,2-dioxaborolan-2-yl)-1H-pyrazole

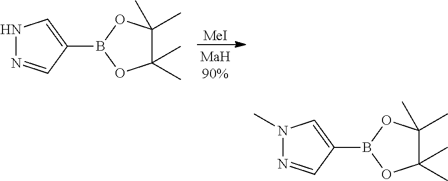

To the solution of 4-pinacolatoboron-1H-pyrazole (1.0 g, 5.0 mmol) in THF (30 mL) was added NaH (0.4 g, 10 mmol). After addition of NaH was completed, to the reaction mixture was added CH₃I (1.42 g, 10 mmol) and stirred overnight at room temperature. The reaction was quenched with MeOH (1 mL). The result mixture was concentrated to give residues, purification by chromatography (EA:PE=1:10) to give compound 1-methyl-4-(4,4,5,5-tetramethyl-1,3,2-dioxaborolan-2-yl)-1H-pyrazole (0.9 g, 90%) as light yellow solid. MS (m/z) (M⁺+H): 209.

II. Preparation of 8-Substituted Pyrazolo[3,4-c]quinoline Derivatives (Scaffold H)

There are two synthesis Routes (Route 1 and Route 2) to prepare key intermediate, 2-(4-(3-acetyl-8-bromo-3H-pyrazolo[3,4-c]quinolin-1-yl)phenyl)-2-methyl propanenitrile (11), which is used for preparing 8-substituted pyrazolo[3,4-c]quinoline derivatives.

The pyrazolo[3,4-c]quinoline derivatives with variation on 8-position were prepared in following six methods.
II-1a. Method 1a Aryl halide 11 coupling with boronic acids
II-1b. Method 1b Aryl halide 11 coupling with boronic esters
II-2. Method 2 Aryl borate 33 coupling with aryl halides
II-3. Method 3 Aryl borate 34 coupling with aryl halides and then ring closure
II-4. Method 4 Modification of pyrazolo[3,4-c]quinoline derivatives
II-5. Method 5 Aryl halide 23 coupling with amines

Route 1. Preparation of Key Intermediate for Scaffold H

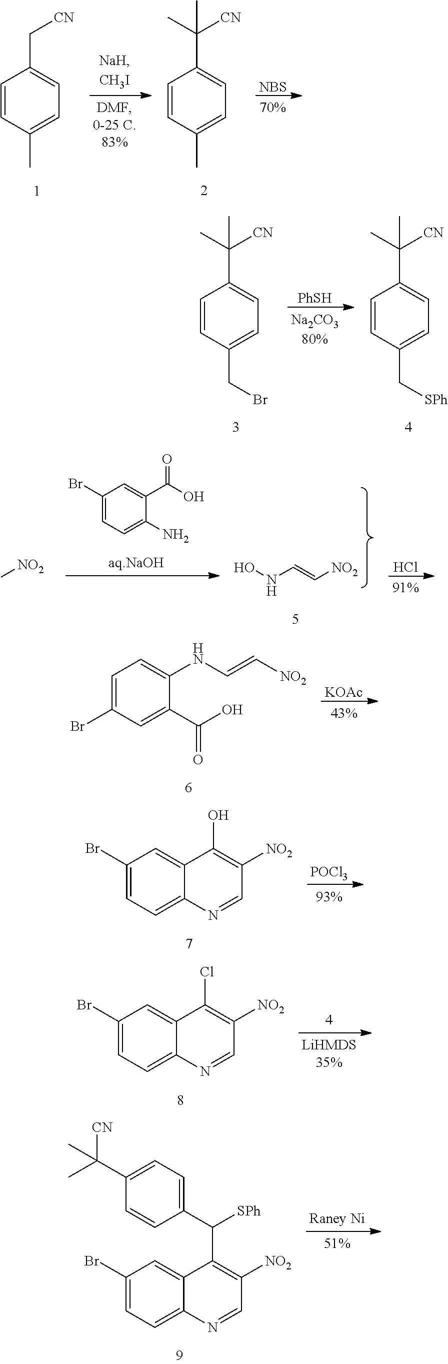

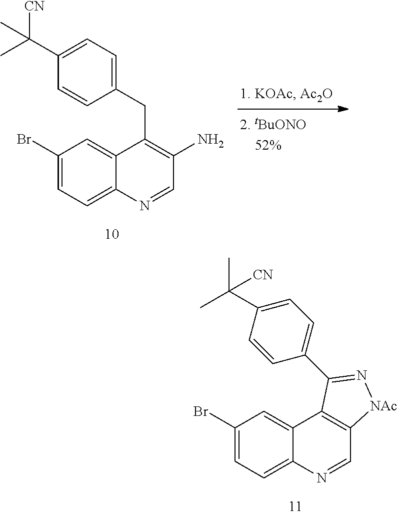

### 2-Methyl-2-p-tolylpropanenitrile (2)

To a solution of 2-p-tolylacetonitrile (65.6 g, 0.5 mol) in DMF (1 L) was added NaH (40 g, 1.0 mol, 60%) at 0° C. After addition of NaH completed, MeI (142 g, 1.0 mol) was added at room temperature. The reaction mixture was stirred overnight at room temperature, quenched with methanol (100 mL), diluted with water (1 L) and extracted with EA (1 L). The organic layer was washed with brine, dried over Na₂SO₄, and concentrated to give 2 (66 g, 83%) as light yellow oil. MS (m/z) (M⁺+H): 160.

### 2-(4-(Bromomethyl)phenyl)-2-methylpropanenitrile (3)

To a solution of compound 2 (32 g, 0.2 mol) in CCl₄ (300 ml) was added NBS (40 g, 0.22 mol) and AIBN (1.64 g, 0.01 mol). The reaction mixture was refluxed for 2 h. After cooled to room temperature, solid was removed by filtration. The filtrate was evaporated to dryness to give crude compound 3 (33 g, 70%) as yellow oil. MS (m/z) (M⁺+H): 238, 240.

### 2-Methyl-2-(4-(phenylthiomethyl)phenyl)propanenitrile (4)

To a solution of compound 3 (1.6 g, 0.0067 mol) in methanol (10 mL) was added Na₂CO₃ (0.7 g, 0.0067 mol) and benzenethiol (0.74 g, 0.0067 mol). The reaction mixture was stirred for 3 h at room temperature. The mixture was diluted with water (20 mL), extracted with EA (30 mL), washed with brine, dried over Na₂SO₄, concentrated, and re-crystallized with methanol to give Compound 4 (1.6 g, 80%). LC-MS (M/Z) M⁺+H): 267; ¹H-NMR (400 MHz, CDCl₃, ppm), δ7.20-7.40 (9H, C₆H₅ and C₆H₄), 4.12(s, 2H, CH₂), 1.71(s, 6H, 2CH₃).

### (E)-N-Hydroxy-2-nitroethenamine (5)

A solution of sodium hydroxide (112 g, 2.8 mol) in water (250 mL) was cooled and stirred at room temperature, to which, nitromethane (61 g, 1.0 mol) was added dropwisely at room temperature and slowly raised to 45° C. for 5 min then cooled to room temperature. Another half amount of nitromethane (61 g, 1.0 mol) was added drop wisely at 45° C. The mixture was stirred for 10 min till clear red solution was obtained. The solution was then heated to 50° C. for 5 min and finally cooled to room temperature, poured onto crashed ice (600 g), and acidified with concentrated hydrogen chloride. The resultant solution of methazonic acid 5 was immediately used for next step.

### (E)-5-Bromo-2-(2-nitrovinylamino)benzoic acid (6)

Compound 5 was immediately added to a filtered solution of 5-bromoanthranilic acid (23.76 g, 0.11 mol) and 500 ml of conc. HCl in 1000 ml water. The solution was allowed to stand at room temperature for 18 hours, and then filtered. The solid product was washed repeatedly with water. The cake was sliced into thin flakes and allowed to dry at room temperature to give compound 6 (26 g, 91%). MS (m/z) (M⁺+H): 287, 289.

### 6-Bromo-3-nitroquinolin-4-ol (7)

Compound 6 (15 g, 0.052 mol) and potassium acetate (6.16 g, 0.063 mol) in acetic anhydride (100 mL) were stirred for 1.5 h at 120° C. The precipitate was filtered and washed with acetic acid until the filtrate was colorless and then with water. The solid was dried to give 7 (6 g, 43%). MS (m/z) (M⁺+H): 269, 271.

### 6-Bromo-4-chloro-3-nitroquinoline (8)

To a solution of 7 (15 g, 0.056 mol) in acetonitrile (80 mL) and DIPEA (15.9 g, 0.123 mol), was added POCl₃ (17.1 g, 0.112 mol) dropwisely at 0° C. The reaction temperature was slowly raised to 100° C. for 2 hours. The mixture was cooled and poured onto ice-water. After Neutralized with aq NaHCO₃, extracted with ethyl acetate, and dried over Na₂SO₄, the crude product was obtained by evaporating of solution to dryness (15 g, 93%) as a brown solid. MS (m/z) (M⁺+H): 287, 289.

### 2-(4-((6-Bromo-3-nitroquinolin-4-yl)(phenylthio)methyl)phenyl)-2-methylpropanenitrile (9)

Compound 8 (0.535 g, 2 mmol) in THF (2 mL) was cooled to −78° C., to which, LHMDS (0.0020 mol) was added drop wise. After the addition completed, the resultant mixture was stirred for 30 min at −78° C. Compound 4 (0.208 g, 1 mmol) in 3 ml THF was add dropwise then stirred for 1 h. The reaction mixture was warmed to room temperature slowly, quenched with NH4Cl, extracted with EA. The organic layer was washed with brine, dried with MgSO₄, filtered, and evaporated. Pure product was obtained from column chromatography (EA:PE=1:20 to 1:10) (0.180 g, 35%) as brown oil. MS (m/z) (M⁺+H): 518, 520.

### 2-(4-((3-Amino-6-bromoquinolin-4-yl)methyl)phenyl)-2-methylpropanenitrile (10)

To a solution of compound 9 (0.052 g, 0.1 mmol) in THF was added Raney-Ni (1.5 g). The reaction mixture was stirred overnight under hydrogen gas atmosphere and filtered. The filtrate was concentrated to dryness to give crude product. Purification through column chromatography (EA:PE=1:6 to 1:3) yielded compound 10 (20 mg, 51%) as a light yellow solid. MS (m/z) (M⁺+H): 380, 382; ¹H-NMR (δ, 400 MHz, CDCl₃, ppm), 8.51 (s, 1H), 7.98 (d, 1H, J=1.83 Hz), 7.87 (d, 1H, J=8.79 Hz), 7.54 (dd, J=1=2.20 Hz, J=2=7.30 Hz), 7.38 (d, 2H, J=8.07 Hz), 7.14 (d, 2H, J=8.07 Hz), 4.24 (s, 2H, NH₂), 3.90 (s, 2H, CH₂) 1.69 (s, 6H, 2CH₃).

### 2-(4-(3-Acetyl-8-bromo-3H-pyrazolo[3,4-c]quinolin-1-yl)phenyl)-2-methyl propanenitrile (11)

To a solution of compound 10 (380 mg, 1 mmol) in toluene (50 mL) was added KOAc (200 mg, 2 mmol) and acetic anhydride (300 mg). The reaction was monitored by HPLC for the consumption of starting material. To the reaction mixture was charged isoamylnitrite (120 mg, 1.2 mmol). The resulting mixture was heated to 80° C. and stirred for 18 h, at which time HPLC of an aliquot indicated the reaction was complete. The solution was concentrated and the residue was purified by column (EA:PE=1:10 to 1:5) to give compound 11 (250 mg, 52%) as a light yellow solid. MS (m/z) (M⁺+H): 432, 434; ¹H-NMR (δ, 400 MHz, DMSO-d6, ppm), 9.92 (s, 1H), 8.18 (d, 1H, J=8.8 Hz), 8.12 (d, 1H, J=2.20 Hz), 7.84-7.95 (m, 4H), 2.84 (s, 3H, Ac), 1.82 (s, 6H, 2CH₃).

Route 2. Preparation of Key Intermediate for Scaffold H

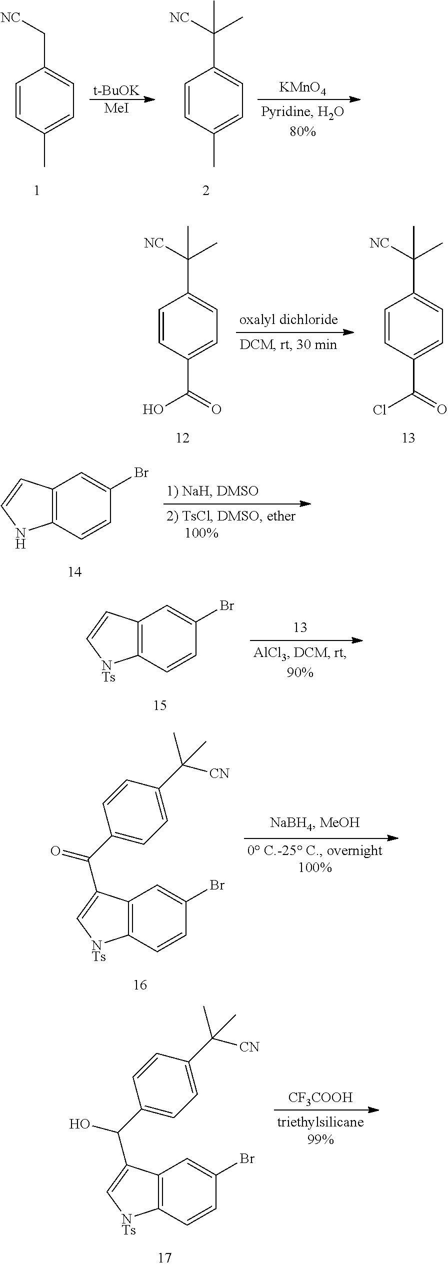

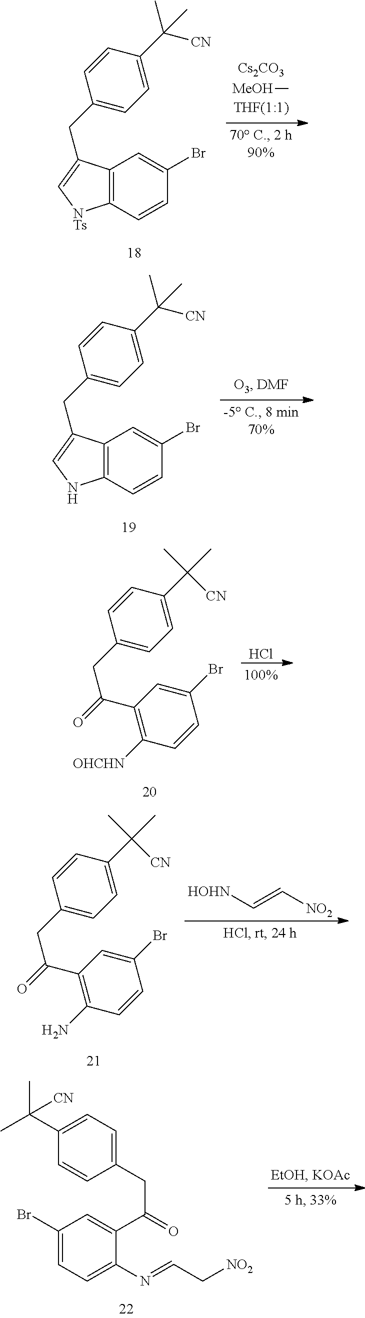

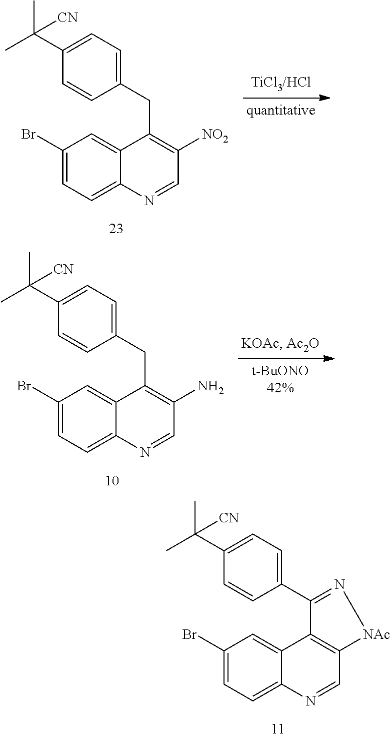

### 4-(2-Cyanopropan-2-yl)benzoic acid (12)

Compound 2 (2 g, 10 mmol) was suspended in a mixture of 10 mL of pyridine and 20 mL of water. KMnO₄ (7.9 g, 50 mmol) was added in one portion. The mixture was heated to reflux for 1 h. Then most pyridine was removed under reduced pressure, and the hot mixture was filtered. The filtrate was acidified with 6 N HCl. The precipitate was collected and dried (1.5 g, 80%).

### 4-(2-Cyanopropan-2-yl)benzoyl chloride (13)

Oxalyl chloride (12.6 g, 0.1 mmol) was added dropwisely to a solution of compound 12 (18 g, 0.1 mmol) in dichloromethane (300 mL). DMF (0.05 mL) as catalyst was dropped into resulting solution. The solution was stirred at room temperature for 30 min. The solvent was removed under reduced pressure to afford acid chloride (20 g) in nearly quantitatively yield and directly utilized in the next step without further purification.

### 5-Bromo-1-tosyl-1H-indole (15)

Sodium hydride (0.44 g, 60% in mineral oil, 11 mmol) was carefully added to a solution of 5-bromo-1H-indole (1.9 g, 10 mmol) in anhydrous dimethylsulfoxide (10 mL) at 0° C. The mixture was stirred at this temperature for 30 minutes. Then p-TsCl (1.9 g, 10 mmol) in anhydrous ether (10 mL) was dropped into the resulting solution at 0° C. and the mixture was stirred at room temperature for one hour. Ether was removed under reduced pressure and the residue was poured onto crashed ice. The product (3.5 g) was collected by filtration as white solid in nearly quantitatively yield, which was used in the next step without further purification. MS (m/z) (M⁺+H) for C₁₅H₁₂BrNO₂S: 350, 352. ¹H-NMR: (δ, ppm, CDCl₃, 400 Hz): 7.86 (dd, 1H), 7.74 (d, 2H), 7.72 (s, 1H), 7.56 (d, 1H), 7.40 (dd, 1H), 7.23 (d, 2H), 6.58 (t, 1H), 2.34 (s, 3H).

### 2-(4-(5-Bromo-1-tosyl-1H-indole-3-carbonyl)phenyl)-2-methylpropanenitrile (16)

To a magnetically stirred suspension of AlCl₃ (1.6 g, 20 mmol) in CH₂Cl₂ (50 mL) at 25° C. was added the acid chloride 13 (1.1 g, 5.7 mmol) in 10 mL of CH₂Cl₂, and the mixture was stirred for 10 min. A solution of compound 15 (2 g, 5.7 mmol) was added dropwise, and the mixture was stirred overnight at 25° C. and quenched with ice. The usual workup and flash chromatography afforded product as white solid (2.7 g) in 90%. MS (m/z) (M⁺+H): for C₂₆H₂₁BrN₂O₃S: 521, 523. ¹H-NMR: (δ, ppm, CDCl₃, 400 Hz): 8.49 (m, 1H), 7.99 (s, 1H), 7.86 (m, 3H), 7.78 (m, 2H), 7.66 (q, 2H), 7.51 (dd, 1H), 7.28 (m, 2H), 2.38 (s, 3H), 1.81 (s, 6H).

### 2-(4-((5-Bromo-1-tosyl-1H-indol-3-yl)(hydroxy)methyl)phenyl)-2-methylpropanenitrile (17)

To a solution of 16 (140 g, 0.26 mol) in MeOH (2 L) was added NaBH₄ (30.6 g, 0.77 mol.) at r.t. The mixture was stirred for 2 h, and concentrated to ca. 200 mL. The mixture was diluted with ice-water (500 mL), and extracted with DCM (2×300 mL). The organic phases were combined, dried over Na₂SO₄, filtered. The filtrate was evaporated to dryness to give compound 17 (128 g, 96%) as a light yellow solid. MS (M/Z) M++H): 523 and 525.

### 2-(4-((5-Bromo-1-tosyl-1H-indol-3-yl)methyl)phenyl)-2-methylpropanenitrile (18)

To a solution of 17 (128 g, 0.25 mol) in DCM (1 L) was added Et₃SiH (142 g, 1.25 mol), CF₃COOH (83 g 0.75 mol) at r.t. The mixture was stirred for 2 hours. To the mixture, was added water (1 L), and the mixture was extracted with DCM (2×200 mL). The organic phases were combined, dried over Na₂SO₄, and filtered. The filtrate was concentrated to give 18 (114 g, 90%) as a light yellow solid. MS (M/Z) M++H): 507, 509.

### 2-(4-((5-Bromo-1-tosyl-1H-indol-3-yl)methyl)phenyl)-2-methylpropanenitrile (18)

Compound 18 can also be prepared through a one-step reaction. To magnetically stirred trifluoroacetic acid (25 mL) at 0° C. under N₂ was added sodium borohydride (30 mmol) over 30 min. To this mixture was added dropwise a solution of compound 16 (0.5 g) in CH₂Cl₂ (25 mL) over 30 min at 15° C. The mixture was stirred overnight at 25° C., diluted with water (75 mL), and neutralized by the addition of sodium hydroxide pellets at 0° C. The layers were separated, the aqueous layer was extracted with CH₂Cl₂, and the usual workup gave 0.48 g (99%) of Compound 18. MS (m/z) (M⁺+H) for C₂₆H₂₃BrN₂O₂S: 507.

### 2-(4-((5-Bromo-1H-indol-3-yl)methyl)phenyl)-2-methylpropanenitrile (19)

Compound 18 (0.3 g, 0.6 mmol) was dissolved in a mixture of THF (5 mL) and MeOH (2.5 mL) at ambient temperature. Cesium carbonate (0.6 g, 1.8 mmol) was added to above solution. The resulting mixture was stirred at 70° C. for 2 h. and the solvent was removed under reduced pressure. To the residue was added water (25 mL). The solid were collected by filtration, washed with water and dried. Yield: 0.2 g, 90%. MS (m/z) (M⁺+H) for C₁₉H₁₇BrN₂: 353.

### N-(4-Bromo-2-(2-(4-(2-cyanopropan-2-yl)phenyl)acetyl)phenyl)formamide (20)

A stream of ozone gas (5%, carried by oxygen) was passed through a solution of compound 19 (150 mg) in DMF (5 m L) at −5° C. for about 8 min until starting material was completely consumed, which was detected by MS. The resulting solution was added to saturated NaCl aqueous solution (30 mL), extracted with EtOAc (30 mL) and dried over Na₂SO₄ to afford the desired product (134 mg) in 70% yield and 85% purity (MS, 214 nm). MS (m/z) (M⁺+H) for C₁₉H₁₇BrN₂O₂: 385, 387. H¹-NMR: (δ, ppm, CDCl₃, 400 Hz): 11.37 (s, 1H), 8.68 (t, 1H), 8.41 (s, 1H), 8.15 (d, 1H), 7.66 (dd, 1H), 7.48 (d, 2H), 7.25 (d, 2H), 4.34 (s, 2H), 1.74 (s, 6H).

### 2-(4-(2-(2-Amino-5-bromophenyl)-2-oxoethyl)phenyl)-2-methylpropanenitrile (21)

A solution of compound 20 (120 mg) was dissolved in acetone (50 mL) and concentrated HCl (3 ml). The mixture was stirred for 24 hours. The reaction was monitored by MS. After the starting material was consumed, the reaction mixture was used in further reaction without further purification. MS (m/z) (M⁺+H): 358.

### (E)-2-(4-(2-(5-Bromo-2-(2-nitroethylideneamino)phenyl)-2-oxoethyl)phenyl)-2-methylpropanenitrile (22)

To a solution of compound 21 in acetone/water, was added fresh-prepared methazonic acid (0.2 g) in water (4 mL) and concentrated hydrochloric acid (3 mL). The reaction mixture was stirred for 24 hours and filtered. The solid was washed with brine and dried by vacuum to give 22 (135 mg, 100% for 2 steps). MS (m/z) (M⁺+H): 429.

### 2-(4-((6-Bromo-3-nitroquinolin-4-yl)methyl)phenyl)-2-methylpropanenitrile (23)

To a solution of compound 22 (4.7 g, 11 mmol) in EtOH (300 mL), was added KOAc (3.3 g, 33 mmol). The reaction mixture was stirred for 5 h at reflux. The reaction mixture was concentrated. The residue was diluted with water and extracted with DCM (3×200 mL). The organic layers were combined, dried over Na₂SO₄, filtered, and concentrated. The residue was purification by silica-gel chromatography (EA:PE=1:10) to give 23 (1.5 g, 33%) as a light yellow solid. MS (m/z) (M⁺+H): 410, 412. H¹-NMR: (δ, ppm, CDCl₃, 400 Hz): 9.31 (s, 1H), 8.25(d, 1H), 8.09 (d, 1H), 7.94 (dd, 1H), 7.39 (m, 2H), 7.12 (m, 2H), 4.68 (s, 2H), 1.69 (s, 6H).

II-1a. Method 1a Aryl Halide 11 Coupling with Bronic Acids

Coupling reactions of aryl halide 11 with boronic acids were carried under three different reaction conditions (Condition a, Condition b, Condition c) and provided final compounds (31). The reaction conditions and results were summarized in the following Table, and the final compounds were summarized in Table II-1a

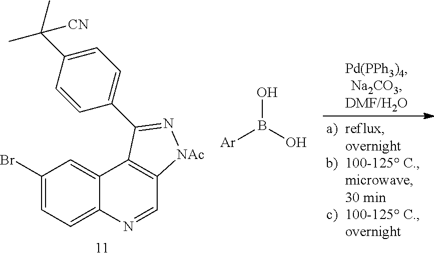

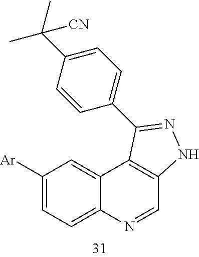

| Entry | ArB(OH)₂ | Method | Yield (%) |
|---|---|---|---|
| 1 | 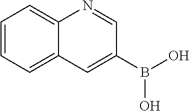 | c | 41 |
| 2 | 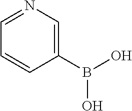 | c a | 43 56 |
| 3 | 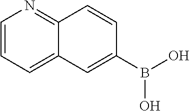 | c | 41 |
| 4 | 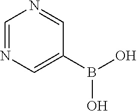 | c | 61 |
| 5 | 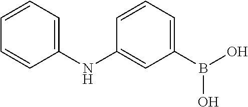 | c | 85 |
| 6 | 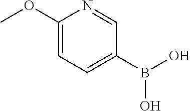 | c | 39 |
| 7 | 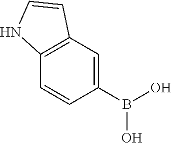 | c | 35 |
| 8 | 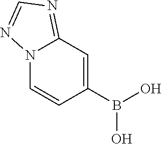 | c | 22 |
| 9 | 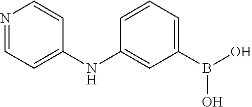 | c | 31 |
| 10 | 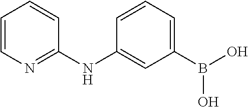 | c | 22 |
| 11 | 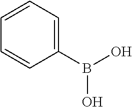 | c | 55 |
| 12 | 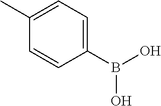 | c | 38 |
| 13 | 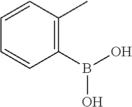 | c | 25 |
| 14 | 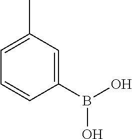 | c | 38 |
| 15 | 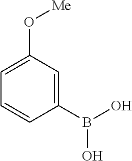 | c | 30 |
| 16 | 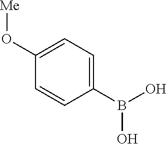 | c | 30 |
| 17 | 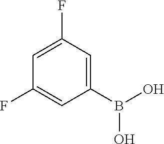 | c | 28 |
| 18 | 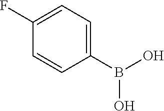 | c | 25 |
| 19 | 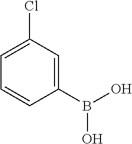 | c | 17 |
| 20 | 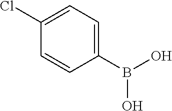 | c | 26 |
| 21 | 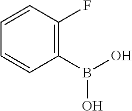 | c | 25 |
| 22 | 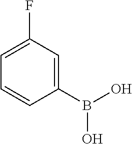 | c | 21 |
| 23 | 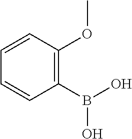 | c | 30 |
| 24 | 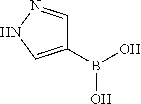 | b | 22 |
| 25 | 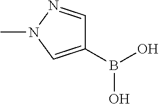 | b | 25 |
| 26 | 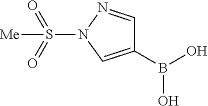 | b | 34 |
| 27 | 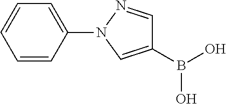 | b | 30 |
| 28 | 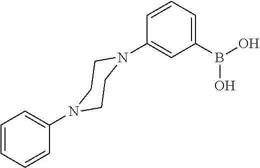 | b | 20 |
| 29 | 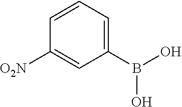 | b | 63 |
| 30 | 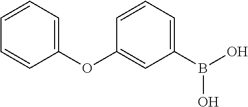 | b | 30 |
| 31 | 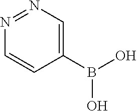 | b | 30 |
| 32 | 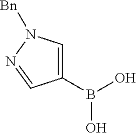 | b | 24 |
| 33 | 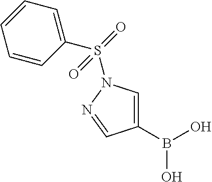 | b | 20 |
| 34 | 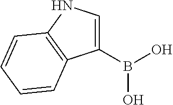 | b | 23 |
| 35 | 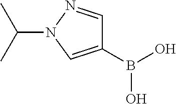 | b | 20 |
| 36 | 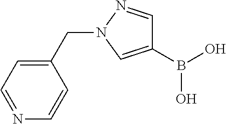 | b | 23 |
| 37 | 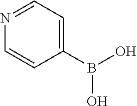 | b | 38 |
| 38 | 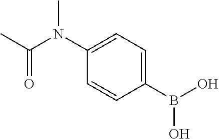 | b | 30 |
| 39 | 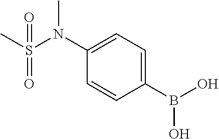 | b | 30 |
| 40 | 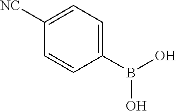 | b | 38 |

TABLE II-1a

| Cpd | | | | MS | |
|---|---|---|---|---|---|
| No. | No | Structure | MF/MW | (M⁺ + H) | IUPAC Name |
| 1078 | 1 | 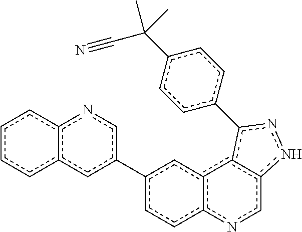 | C29H21N5/ 439.5 | 440 | 2-methyl-2-(4-(8-(quinolin- 3-yl)-3H-pyrazolo[3,4- c]quinolin-1- yl)phenyl)propanenitrile |
| 1107 | 2 | 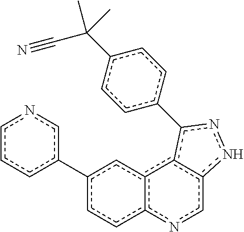 | C25H19N5/ 389.5 | 390 | 2-methyl-2-(4-(8-(pyridin-3- yl)-3H-pyrazolo[3,4- c]quinolin-1- yl)phenyl)propanenitrile |
| 1110 | 3 | 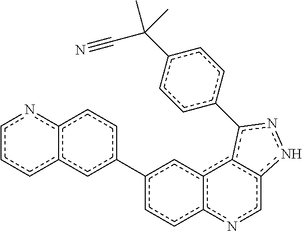 | C29H21N5/ 439.5 | 439 | 2-methyl-2-(4-(8-(quinolin- 6-yl)-3H-pyrazolo[3,4- c]quinolin-1- yl)phenyl)propanenitrile |
| 1108 | 4 | 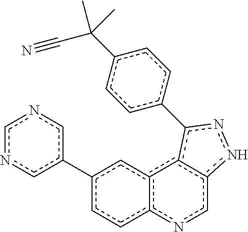 | C24H18N6/ 390.4 | 391 | 2-methyl-2-(4-(8- (pyrimidin-5-yl)-3H- pyrazolo[3,4-c]quinolin-1- yl)phenyl)propanenitrile |
| 1119 | 5 | 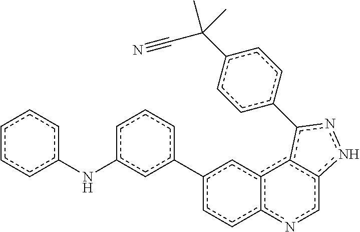 | C32H25N5/ 479.6 | 480 | 2-methyl-2-(4-(8-(3- (phenylamino)phenyl)-3H- pyrazolo[3,4-c]quinolin-1- yl)phenyl)propanenitrile |
| 1118 | 6 | 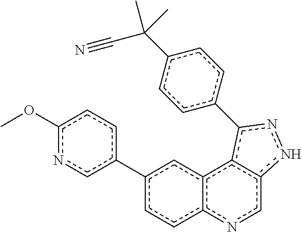 | C26H21N5O/ 419.5 | 420 | 2-(4-(8-(6-methoxypyridin- 3-yl)-3H-pyrazolo[3,4- c]quinolin-1-yl)phenyl)-2- methylpropanenitrile |
| 1114 | 7 | 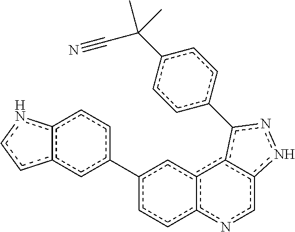 | C28H21N5/ 427.5 | 428 | 2-(4-(8-(1H-indol-5-yl)-3H- pyrazolo[3,4-c]quinolin-1- yl)phenyl)-2- methylpropanenitrile |
| 1111 | 8 | 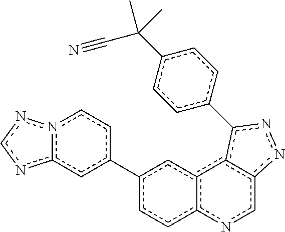 | C26H19N7/ 429.5 | 430 | 2-(4-(8-[1,2,4]triazolo[1,5- a]pyridin-7-yl)-3H- pyrazolo[3,4-c]quinolin-1- yl)phenyl)-2- methylpropanenitrile |
| 1121 | 9 | 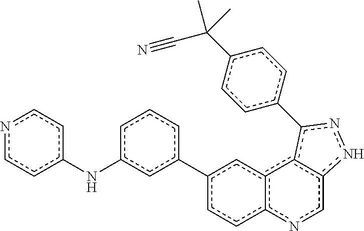 | C31H24N6/ 480.6 | 481 | 2-methyl-2-(4-(8-(3- (pyridin-4-ylamino)phenyl)- 3H-pyrazolo[3,4-c]quinolin- 1-yl)phenyl)propanenitrile |
| 1119 | 10 | 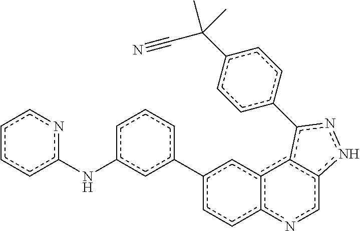 | C31H24N6/ 480.6 | 481 | 2-methyl-2-(4-(8-(3- (pyridin-2-ylamino)phenyl)- 3H-pyrazolo[3,4-c]quinolin- 1-yl)phenyl)propanenitrile |
| 1125 | 11 | 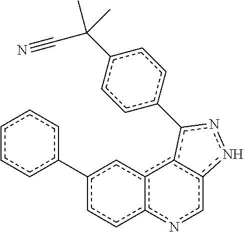 | C26H20N4/ 388.5 | 389 | 2-methyl-2-(4-(8-phenyl- 3H-pyrazolo[3,4-c]quinolin- 1-yl)phenyl)propanenitrile |
| 1126 | 12 | 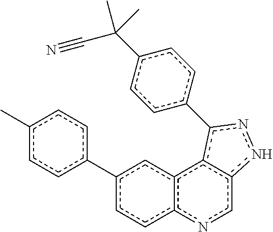 | C27H22N4/ 402.5 | 403 | 2-methyl-2-(4-(8-p-tolyl- 3H-pyrazolo[3,4-c]quinolin- 1-yl)phenyl)propanenitrile |
| 1124 | 13 | 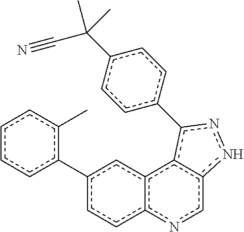 | C27H22N4/ 402.5 | 403 | 2-methyl-2-(4-(8-o-tolyl- 3H-pyrazolo[3,4-c]quinolin- 1-yl)phenyl)propanenitrile |
| 1123 | 14 | 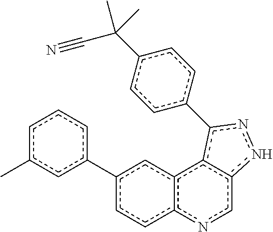 | C27H22N4/ 402.5 | 403 | 2-methyl-2-(4-(8-m-tolyl- 3H-pyrazolo[3,4-c]quinolin- 1-yl)phenyl)propanenitrile |
| 1115 | 15 | 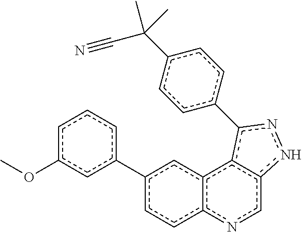 | C27H22N4O/ 418.5 | 419 | 2-(4-(8-(3-methoxyphenyl)- 3H-pyrazolo[3,4-c]quinolin- 1-yl)phenyl)-2- methylpropanenitrile |
| 1117 | 16 | 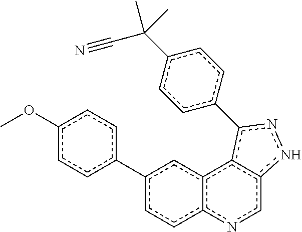 | C27H22N4O/ 418.5 | 419 | 2-(4-(8-(4-methoxyphenyl)- 3H-pyrazolo[3,4-c]quinolin- 1-yl)phenyl)-2- methylpropanenitrile |
| 1112 | 17 | 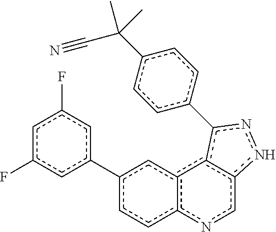 | C26H18F2N4/ 424.4 | 425 | 2-(4-(8-(3,5- difluorophenyl)-3H- pyrazolo[3,4-c]quinolin-1- yl)phenyl)-2- methylpropanenitrile |
| 1116 | 18 | 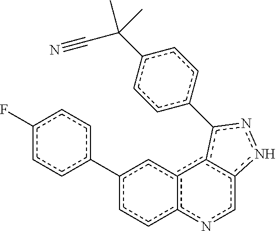 | C26H19FN4/ 406.5 | 407 | 2-(4-(8-(4-fluorophenyl)- 3H-pyrazolo[3,4-c]quinolin- 1-yl)phenyl)-2- methylpropanenitrile |
| 1113 | 19 | 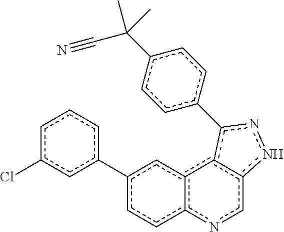 | C26H19ClN4/ 422.9 | 423 | 2-(4-(8-(3-chlorophenyl)- 3H-pyrazolo[3,4-c]quinolin- 1-yl)phenyl)-2- methylpropanenitrile |
| 1162 | 20 | 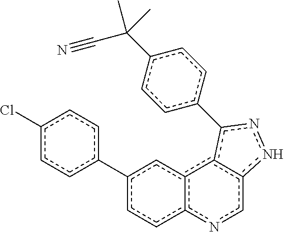 | C26H19ClN4/ 422.9 | 423 | 2-(4-(8-(4-chlorophenyl)- 3H-pyrazolo[3,4-c]quinolin- 1-yl)phenyl)-2- methylpropanenitrile |
| 1163 | 21 | 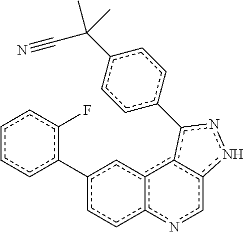 | C26H19FN4/ 406.5 | 407 | 2-(4-(8-(2-fluorophenyl)- 3H-pyrazolo[3,4-c]quinolin- 1-yl)phenyl)-2- methylpropanenitrile |
| 1164 | 22 | 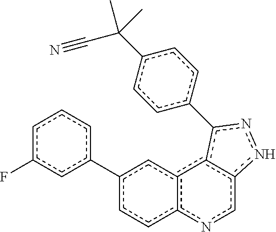 | C26H19FN4/ 406.5 | 407 | 2-(4-(8-(3-fluorophenyl)- 3H-pyrazolo[3,4-c]quinolin- 1-yl)phenyl)-2- methylpropanenitrile |
| 1165 | 23 | 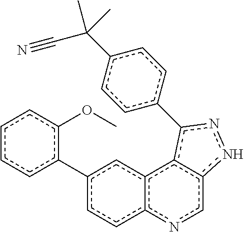 | C27H22N4O/ 418.5 | 419 | 2-(4-(8-(2-methoxyphenyl)- 3H-pyrazolo[3,4-c]quinolin- 1-yl)phenyl)-2- methylpropanenitrile |
| 1166 | 24 | 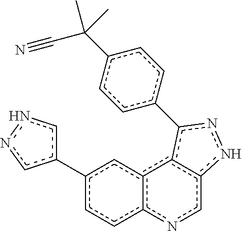 | C23H18N6/ 378.4 | 379 | 2-(4-(8-(1H-pyrazol-4-yl)- 3H-pyrazolo[3,4-c]quinolin- 1-yl)phenyl)-2- methylpropanenitrile |
| 1167 | 25 | 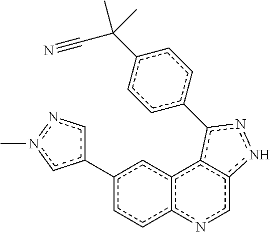 | C24H20N6/ 392.5 | 393 | 2-methyl-2-(4-(8-(1-methyl- 1H-pyrazol-4-yl)-3H- pyrazolo[3,4-c]quinolin-1- yl)phenyl)propanenitrile |
| 1168 | 26 | 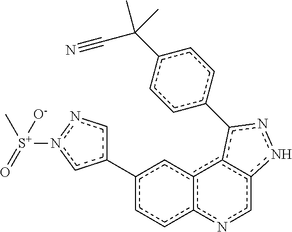 | C24H20N6O2S/ 456.5 | 457 | 2-methyl-2-(4-(8-(1- (methylsulfonyl)-1H- pyrazol-4-yl)-3H- pyrazolo[3,4-c]quinolin-1- yl)phenyl)propanenitrile |
| 1169 | 27 | 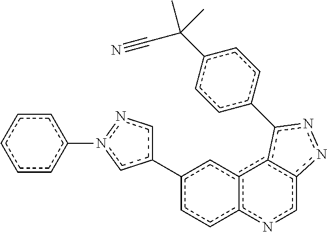 | C29H22N6/ 454.5 | 455 | 2-methyl-2-(4-(8-(1-phenyl- 1H-pyrazol-4-yl)-3H- pyrazolo[3,4-c]quinolin-1- yl)phenyl)propanenitrile |
| 1170 | 28 | 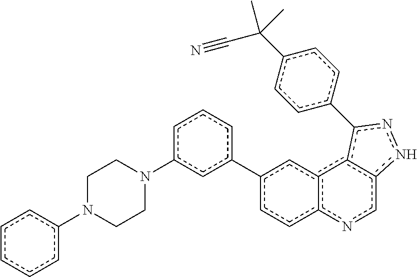 | C36H32N6/ 548.7 | 549 | 2-methyl-2-(4-(8-(3-(4- phenylpiperazin-1- yl)phenyl)-3H-pyrazolo[3,4- c]quinolin-1- yl)phenyl)propanenitrile |
| 1180 | 29 | 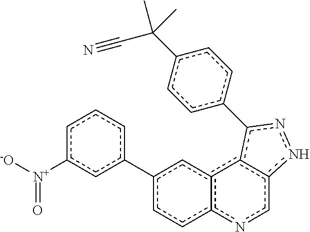 | C26H19N5O2/ 433.5 | 434 | 2-methyl-2-(4-(8-(3- nitrophenyl)-3H- pyrazolo[3,4-c]quinolin-1- yl)phenyl)propanenitrile |
| 1181 | 30 | 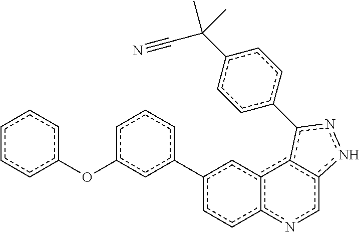 | C32H24N4O/ 480.6 | 481.3 | 2-methyl-2-(4-(8-(3- phenoxyphenyl)-3H- pyrazolo[3,4-c]quinolin-1- yl)phenyl)propanenitrile |
| 1182 | 31 | 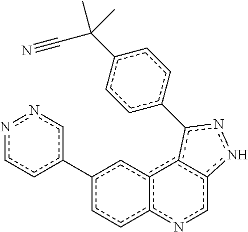 | C24H18N6/ 390.4 | 391.4 | 2-methyl-2-(4-(8- (pyridazin-4-yl)-3H- pyrazolo[3,4-c]quinolin-1- yl)phenyl)propanenitrile |
| 1183 | 32 | 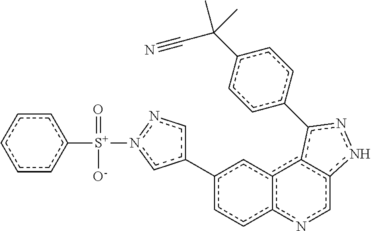 | C29H22N6O2S/ 518.6 | 519 | 2-methyl-2-(4-(8-(1- (phenylsulfonyl)-1H- pyrazol-4-yl)-3H-pyrazolo[3,4-c]quinolin-1- yl)phenyl)propanenitrile |
| 1184 | 33 | 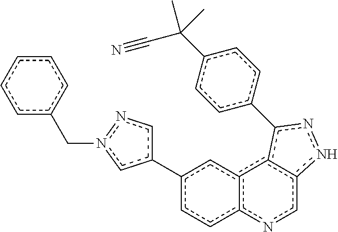 | C30H24N6/ 468.6 | 469 | 2-(4-(8-(1-benzyl-1H- pyrazol-4-yl)-3H- pyrazolo[3,4-c]quinolin-1- yl)phenyl)-2- methylpropanenitrile |
| 1185 | 34 | 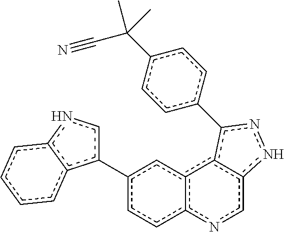 | C28H21N5/ 427.5 | 428 | 2-(4-(8-(1H-indol-3-yl)-3H- pyrazolo[3,4-c]quinolin-1- yl)phenyl)-2- methylpropanenitrile |
| 1186 | 35 | 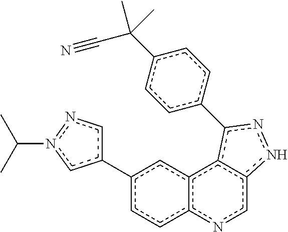 | C26H24N6/ 420.5 | 421 | 2-(4-(8-(1-isopropyl-1H- pyrazol-4-yl)-3H- pyrazolo[3,4-c]quinolin-1- yl)phenyl)-2- methylpropanenitrile |
| 1187 | 36 | 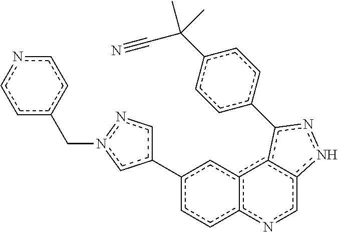 | C29H23N7/ 469.5 | 470 | 2-methyl-2-(4-(8-(1- (pyridin-4-ylmethyl)-1H- pyrazol-4-yl)-3H- pyrazolo[3,4-c]quinolin-1- yl)phenyl)propanenitrile |
| 1188 | 37 | 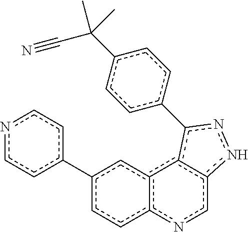 | C25H19N5/ 389.5 | 390 | 2-methyl-2-(4-(8-(pyridin-4- yl)-3H-pyrazolo[3,4- c]quinolin-1- yl)phenyl)propanenitrile |
| 1189 | 38 | 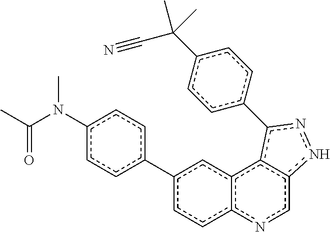 | C29H25N5O/ 459.5 | 460 | N-(4-(1-(4-(2-cyanopropan- 2-yl)phenyl)-3H- pyrazolo[3,4-c]quinolin-8- yl)phenyl)-N- methylacetamide |
| 1190 | 39 | 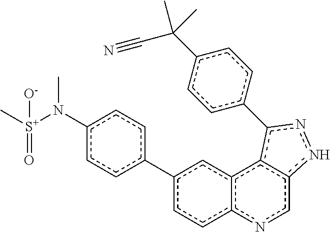 | C28H25N5O2S/ 495.6 | 496 | N-(4-(1-(4-(2-cyanopropan- 2-yl)phenyl)-3H- pyrazolo[3,4-c]quinolin-8- yl)phenyl)-N- methylmethanesulfonamide |
| 1191 | 40 | 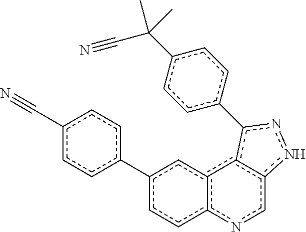 | C27H19N5/ 413.5 | 414.2 | 4-(1-(4-(2-cyanopropan-2- yl)phenyl)-3H-pyrazolo[3,4- c]quinolin-8-yl)benzonitrile |

Synthetic Procedures for Preparing Compounds in Table II-1a.

### General Procedure for Condition a (Example 2)

To a solution of 11, 2-(4-(3-acetyl-8-bromo-3H-pyrazolo[3,4-c]quinolin-1-yl)phenyl)-2-methylpropanenitrile (90 mg, 0.2 mmol) in DMF (15 ml) was added building block as boronic acid (1.0 mmol), 1M Na₂CO₃ (138 mg, 1.0 mmol, in 1 mL water) and Pd(PPh₃)₄ (11 mg, 0.01 mmol). The mixture was purged with nitrogen gas several times and stirred overnight at 100-120° C. The mixture was diluted with water (20 mL) and extracted with DCM (50 mL×3). The organic phases were combined, washed with brine, dried over Na₂SO₄, filtered, and concentrated. The residue was purified through silica gel column eluted with DCM:Methanol 50:1 to 30:1. The obtained solid was re-crystallized from methanol/ether to give product Example 2 as pale yellow powder (30 mg, 43%).

### General Procedure for Condition b (Example 20)

To a solution of 11 (90 mg, 0.2 mmol) in DMF (4 mL) was added 2-fluorophenylboronic acid (1 mmol), 1M Na₂CO₃ (100 mg, 0.6 mmol, in 0.6 mL water) and Pd(PPh₃)₄ (22 mg, 0.1 mmol). The reaction mixture was purged with nitrogen and stirred under microwave for 30 min at 105-120° C. The reaction mixture was diluted with water (10 mL) and extracted with DCM (3×20 mL). The organic layer was washed with brine, dried over Na₂SO₄, filtered. The filtrate was concentrated. The resulting residue was purified by column chromatography (DCM:Methanol 80:1 to 60:1) to give Example 20 (16 mg, 26%) as a light yellow solid.

### General Procedure for Condition c (Example 2)

To a solution of 2-(4-(3-acetyl-8-bromo-3H-pyrazolo[3,4-c]quinolin-1-yl)phenyl)-2-methylpropanenitrile (90 mg, 0.2 mmol) in DMF (15 ml) was added building block as boronic acid (1.0 mmol), 1M Na₂CO₃ (138 mg, 1.0 mmol, in 1 mL water) and Pd(PPh₃)₄ (11 mg, 0.01 mmol). The mixture was purged with nitrogen gas several times and stirred overnight at 100-120° C. The mixture was diluted with water (20 mL) and extracted with DCM (50 mL×3). The organic phases were combined, washed with brine, dried over Na₂SO₄, filtered, and concentrated. The residue was purified through silica gel column eluted with DCM:Methanol 50:1 to 30:1. The obtained solid was re-crystallized from methanol/ether to give product Example 2 as pale yellow powder (30 mg, 43%).

### Example 1

### 2-methyl-2-(4-(8-(quinolin-3-yl)-3H-pyrazolo[3,4-c]quinolin-1-yl)phenyl)propanenitrile (1078)

29 mg offwhite powder, 41% yield. MS (m/z) (M⁺+H): 440. ¹H-NMR: (δ, ppm, DMSO-d6, 400 Hz) 14.3 (s, 1H), 9.34 (s, 1H), 9.19 (d, 1H, J=2.18 Hz), 8.55 (d, 1H, J=2.18 Hz), 8.49 (s, 1H), 8.27-8.31 (m, 1H), 8.13-8.19 (m, 1H), 8.04-8.07 (m, 2H), 7.97-7.99 (m, 2H), 7.84-7.86 (m, 2H), 7.76-7.80 (m, 2H), 7.63-7.67 (m, 2H), 1.75-1.81 (m, 6H, 2CH3).

### Example 2

### 2-methyl-2-(4-(8-(pyridin-3-yl)-3H-pyrazolo[3,4-c]quinolin-1-yl)phenyl)propanenitrile (1107)

30 mg brown powder, 43% yield. (MS (m/z) M⁺+H): 390. ¹H-NMR (δ, ppm, DMSO-d6, 300 MHz): 14.26 (s, 1H, NH), 9.34 (s, 1H), 8.83 (d, 1H, J=2.2 Hz), 8.57 (d, 1H, J=4.76 Hz), 8.29(d, 1H, J=1.83 Hz), 8.24 (d, 1H, J=8.43 Hz), 7.97-8.03 (m, 2H), 7.91 (d, 2H, J=8.43 Hz), 7.82 (d, 2H, J=8.07 Hz), 7.44-7.47 (m, 1H), 1.78 (s, 6H).

### Example 3

### 2-methyl-2-(4-(8-(quinolin-6-yl)-3H-pyrazolo[3,4-c]quinolin-1-yl)phenyl)propanenitrile (1110)

41% yield. MS (m/z) (M⁺+H): 439. ¹H-NMR: (δ, ppm, DMSO-d6, 400 Hz) 14.29 (s, 1H), 9.34 (s, 1H), 8.91-8.94 (m, 1H), 7.86-8.50 (m, 11H), 7.52-7.60 (m, 1H), 1.80 (s, 6H).

### Example 4

### 2-methyl-2-(4-(8-(pyrimidin-5-yl)-3H-pyrazolo[3,4-c]quinolin-1-yl)phenyl)propanenitrile (1108)

61% yield. MS (m/z) (M⁺+H): 391. ¹H-NMR: (δ, ppm, DMSO-d6, 400 Hz) 14.32 (s, 1H), 9.34 (s, 1H), 9.20 (s, 1H), 9.05 (d, 2H, J=4.39 Hz), 8.33 (s, 1H), 8.28 (d, 1H, J=8.78 Hz), 8.08 (dd, J=11.95 Hz, J=27.10 Hz), 7.93 (d, 2H, J=8.29 Hz), 7.78 (d, 2H, J=7.81 Hz).

### Example 5

### 2-methyl-2-(4-(8-(3-(phenylamino)phenyl)-3H-pyrazolo[3,4-c]quinolin-1-yl)phenyl)propanenitrile (1119)

85% yield. MS (m/z) (M⁺+H): 480. ¹H-NMR: (δ, ppm, DMSO-d6, 400 Hz) 14.23 (s, 1H), 9.28 (s, 1H), 8.31 (s, 2H), 8.20 (d, 2H, J=8.30 Hz), 7.83-7.89 (m, 3H), 7.71 (m, 2H, J=7.81 Hz), 7.21-7.31 (m, 4H), 7.02-7.11 (m, 4H), 6.82-6.86 (m, 1H), 1.68 (s, 6H).

### Example 6

### 2-(4-(8-(6-methoxypyridin-3-yl)-3H-pyrazolo[3,4-c]quinolin-1-yl)phenyl)-2-methylpropanenitrile

39% yield. MS (m/z) (M⁺+H): 420. ¹H-NMR: (δ, ppm, DMSO-d6, 400 Hz) 14.23 (s, 1H), 9.27 (s, 1H), 8.51 (s, 1H), 8.40 (s, 1H), 8.14-8.17 (m, 2H), 7.77-7.92 (m, 5H), 6.86 (d, 1H, J=8.52 Hz), 6.74 (d, 1H, J=8.24 Hz), 3.86 (s, 3H), 1.79 (s, 6H).

### Example 7

### 2-(4-(8-(1H-indol-5-yl)-3H-pyrazolo[3,4-c]quinolin-1-yl)phenyl)-2-methylpropanenitrile (1114)

35% yield. MS (m/z) (M⁺+H): 428. ¹H-NMR: (δ, ppm, DMSO-d6, 400 MHz) 11.23 (s, 1H), 9.17 (s, 1H), 8.38 (s, 1H), 8.06 (d, 1H), 7.91 (d, 2H, J=8.29 Hz), 7.87 (d, 1H), 7.79 (s, 1H), 7.77 (d, 2H, J=8.29 Hz), 7.44 (2H, J=8.28 Hz), 7.36-7.37 (m, 2H), 6.46 (s, 1H), 1.62 (s, 6H).

### Example 8

### 2-(4-(8-([1,2,4]triazolo[1,5-a]pyridin-7-yl)-3H-pyrazolo[3,4-c]quinolin-1-yl)phenyl)-2-methylpropanenitrile

22% yield. MS (m/z) (M⁺+H): 430. ¹H-NMR: (δ, ppm, DMSO-d6, 400 Hz) 14.30 (s, 1H), 9.33 (s, 1H), 9.29 (s, 1H), 8.57 (s, 1H), 8.36 (s, 1H), 8.24 (d, 1H, J=8.52 Hz), 8.10 (d, 1H, J=8.52 Hz), 7.81-7.97 (m, 6H), 1.81 (s, 6H).

### Example 9

### 2-methyl-2-(4-(8-(3-(pyridin-4-ylamino)phenyl)-3H-pyrazolo[3,4-c]quinolin-1-yl)phenyl)propanenitrile (111a)

31% yield. MS (m/z) (M⁺+H): 481. ¹H-NMR: (δ, ppm, DMSO-d6, 400 Hz) 9.31 (s, 1H), 9.12 (s, 1H), 8.94 (s, 1H), 8.34 (s, 1H), 8.19-8.21 (m, 3H), 7.91-7.96 (m, 2H), 7.75 (d, 2H, J=6.97 Hz), 7.31 (s, 1H), 7.30 (d, 1H, J=7.34 Hz), 7.25 (d, 1H, J=7.33 Hz), 7.13 (t, 1H, J=7.70 Hz), 6.95 (d, 2H, J=25.66 Hz), 6.63 (s, 1H), 6.47 (d, 1H, J=7.33 Hz), 1.68 (s, 6H).

### Example 10

### 2-methyl-2-(4-(8-(3-(pyridin-2-ylamino)phenyl)-3H-pyrazolo[3,4-c]quinolin-1-yl)phenyl)propanenitrile (1119)

22% yield. MS (m/z) (M⁺+H): 481. ¹H-NMR: (δ, ppm, DMSO-d6, 400 Hz) 14.24 (s, 1H), 9.29 (s, 1H), 9.17 (s, 1H), 8.92 (s, 1H), 7.51-8.33 (m, 8H), 7.31 (t, 1H, J=7.80 Hz), 7.21 (t, 1H, J=7.8 Hz), 7.10 (d, 1H, J=7.32 Hz), 6.67-6.87 (m, 3H), 1.62 (s, 6H).

### Example 11

### 2-methyl-2-(4-(8-phenyl-3H-pyrazolo[3,4-c]quinolin-1-yl)phenyl)propanenitrile (1125)

55% yield. MS (m/z) (M⁺+H): 389. ¹H-NMR: (δ, ppm, MeOH-D4+DMSO-d6, 400 Hz) 9.18 (s, 1H), 8.24 (s, 1H), 8.12 (d, 1H, J=8.77 Hz), 7.88 (d, 1H, J=8.77 Hz), 7.82 (d, 2H, J=8.29 Hz), 7.73 (d, 2H, J=8.28 Hz), 7.52 (d, 2H, J=7.31 Hz), 7.26-7.37 (m, 3H).

### Example 12

### 2-methyl-2-(4-(8-p-tolyl-3H-pyrazolo[3,4-c]quinolin-1-yl)phenyl)propanenitrile (1126)

38% yield. MS (m/z) M⁺+H): 403. ¹H-NMR: (δ, ppm, MeOH-D4+DMSO-d6, 400 Hz) 9.23 (s, 1H), 8.27 (d, 1H, J=1.95 Hz), 8.17 (d, 1H, J=8.77 Hz), 7.92 (dd, 1H, J=11.95 Hz, J=28.77 Hz), 7.89 (d, 2H, J=8.29 Hz), 7.78 (d, 2H, J=8.29 Hz), 7.38 (d, 2H, J=8.29 Hz), 7.22 (d, 2H, J=8.29 Hz), 2.30 (s, 3H), 1.78 (s, 6H).

### Example 13

### 2-methyl-2-(4-(8-o-tolyl-3H-pyrazolo[3,4-c]quinolin-1-yl)phenyl)propanenitrile (1124)

25% yield. MS (m/z) (M⁺+H): 403. ¹H-NMR: (δ, ppm, DMSO-d6, 400 Hz) 14.24 (s, 1H), 9.32 (s, 1H), 8.20 (d, 1H, J=8.29 Hz), 8.01 (s, 1H), 7.84 (d, 2H, J=8.29 Hz), 7.66-7.76 (m, 3H), 7.27 (s, 4H), 2.22 (s, 3H), 1.73 (s, 6H).

### Example 14

### 2-methyl-2-(4-(8-m-tolyl-3H-pyrazolo[3,4-c]quinolin-1-yl)phenyl)propanenitrile (1123)

38% yield. MS (m/z) (M⁺+H): 403. ¹H-NMR: (δ, ppm, MeOH-D4+DMSO-d6, 400 Hz) 9.26 (s, 1H), 8.33 (s, 1H), 8.16 (s, 1H), 7.91 (brs, 3H), 7.80 (brs, 2H), 7.41 (brs, 2H), 7.30 (s, 1H), 7.17 (s, 1H), 2.34 (s, 3H), 1.77 (s, 6H).

### Example 15

### 2-(4-(8-(3-methoxyphenyl)-3H-pyrazolo[3,4-c]quinolin-1-yl)phenyl)-2-methylpropanenitrile (1115)

30% yield. MS (m/z) (M⁺+H): 419. ¹H-NMR: (δ, ppm, DMSO-d6, 400 Hz) 14.26 (s, 1H), 9.30 (s, 1H), 8.32 (s, 1H), 8.19 (d, 1H, J=7.80 Hz), 7.85-7.98 (m, 3H), 7.79 (d, 2H, J=7.31 Hz), 7.36 (t, 1H, J=7.80 Hz), 7.20(d, 1H, J=7.80 Hz), 7.15 (s, 1H), 3.82 (s, 3H), 1.79 (s, 6H).

### Example 16

### 2-(4-(8-(4-methoxyphenyl)-3H-pyrazolo[3,4-c]quinolin-1-yl)phenyl)-2-methylpropanenitrile (1117)

30% yield. MS (m/z) (M⁺+H): 419. ¹H-NMR: (δ, ppm, MeOH-D4+DMSO-d6, 400 Hz) 9.20 (s, 1H), 8.20 (s, 1H), 8.13 (d, 1H, J=8.77 Hz), 7.80-7.88 (m, 3H), 7.76 (d, 2H, J=8.29 Hz), 7.50 (d, 2H, J=8.29 Hz), 7.08 (d, 2H, J=8.77 Hz), 3.75 (s, 3H), 1.78 (s, 6H).

### Example 17

### 2-(4-(8-(3,5-difluorophenyl)-3H-pyrazolo[3,4-c]quinolin-1-yl)phenyl)-2-methylpropanenitrile (1112)

28% yield. MS (m/z) (M⁺+H): 425. ¹H-NMR: (δ, ppm, DMSO-d6, 400 Hz) 14.27 (s, 1H), 9.31 (s, 1H), 8.26 (s, 1H), 8.18 (d, 1H, J=8.30 Hz), 8.10 (d, 1H, J=7.32 Hz), 7.58-8.00 (m, 4H), 7.28 (d, 3H), 1.79 (s, 6H).

### Example 18

### 2-(4-(8-(4-fluorophenyl)-3H-pyrazolo[3,4-c]quinolin-1-yl)phenyl)-2-methylpropanenitrile (1116)

25% yield. MS (m/z) (M⁺+H): 407. ¹H-NMR: (δ, ppm, DMSO-d6, 400 MHz): 14.77 (s, 1H), 9.29 (s, 1H), 8.25 (s, 1H), 8.23 (d, 1H, J=15.12 Hz), 7.84-7.95 (m, 3H), 7.74-7.79 (m, 2H), 7.58-7.67 (m, 2H), 7.27 (t, 2H, J=8.73 Hz), 1.80 (s, 6H).

### Example 19

### 2-(4-(8-(3-chlorophenyl)-3H-pyrazolo[3,4-c]quinolin-1-yl)phenyl)-2-methylpropanenitrile (1113)

17% yield. MS (m/z) (M⁺+H): 423. ¹H-NMR: (δ, ppm, DMSO-d6, 400 Hz) 14.33 (s, 1H), 9.31 (s, 1H), 8.32 (s, 1H), 8.20 (d, 1H, J=8.78 Hz), 7.89-7.94 (m, 3H), 7.74-7.80 (m, 2H), 7.60-7.63 (m, 2H), 7.43-7.48 (m, 2H), 1.80 (s, 6H).

### Example 20

### 2-(4-(8-(4-chlorophenyl)-3H-pyrazolo[3,4-c]quinolin-1-yl)phenyl)-2-methylpropanenitrile (1162)

26% yield. MS (m/z) (M⁺+H): 423. ¹H-NMR: (δ, ppm, DMSO-d6, 400 Hz) 9.32 (s, 1H), 8.31 (s, 1H), 8.24 (d, 1H, J=8.43 Hz), 7.98(d, 1H, J=8.43 Hz), 7.93(d, 2H, J=8.06 Hz), 7.82(d, 2H, J=8.43 Hz), 7.65(d, 2H, J=8.43 Hz), 7.52(d, 2H, J=8.80 Hz), 1.80(s, 6H).

### Example 21

### 2-(4-(8-(2-fluorophenyl)-3H-pyrazolo[3,4-c]quinolin-1-yl)phenyl)-2-methylpropanenitrile (1163)

25% yield. MS (m/z) (M⁺+H): 407. ¹H-NMR: (δ, ppm, DMSO-d6, 400 Hz) 14.28 (s, 1H), 9.33 (s, 1H), 8.29 (s, 1H), 8.24 (d, 1H, J=8.77 Hz), 7.74-7.87 (m, 5H), 7.55-7.59 (m, 1H), 7.43 (brs, 1H), 7.28-7.32 (m, 2H), 1.77 (s, 6H).

### Example 22

### 2-(4-(8-(3-fluorophenyl)-3H-pyrazolo[3,4-c]quinolin-1-yl)phenyl)-2-methylpropanenitrile (1164)

21% yield. MS (m/z) (M⁺+H): 407. ¹H-NMR: (δ, ppm, DMSO-d6, 400 Hz) 14.27 (s, 1H), 9.32 (s, 1H), 8.31 (s, 1H), 8.23 (d, 1H, J=8.28 Hz), 8.02 (d, 1H, J=8.28 Hz), 7.93 (d, 2H, J=7.80 Hz), 7.82 (d, 2H, J=8.28 Hz), 7.44-7.51 (m, 3H), 7.10 (m, 1H), 1.80 (s, 6H).

### Example 23

### 2-(4-(8-(2-methoxyphenyl)-3H-pyrazolo[3,4-c]quinolin-1-yl)phenyl)-2-methylpropanenitrile (1165)

30% yield. MS (m/z) (M⁺+H): 419. ¹H-NMR: (δ, ppm, DMSO-d6, 400 Hz) 14.23 (s, 1H), 9.29 (s, 1H), 8.35 (s, 1H), 8.17 (d, 1H, J=8.78 Hz), 7.87 (d, 2H, J=7.8 Hz), 7.73-7.79 (m, 3H), 7.36 (t, 2H, J=7.31 Hz), 7.11 (d, 1H, J=8.29 Hz), 7.03 (t, 1H, J=7.31 Hz), 3.74 (s, 2H), 1.80 (s, 2H).

### Example 24

### 2-(4-(8-(1H-pyrazol-4-yl)-3H-pyrazolo[3,4-c]quinolin-1-yl)phenyl)-2-methylpropanenitrile (1166)

22% yield. MS (m/z) (M⁺+H): 379. ¹H-NMR: (δ, ppm, DMSO-d6, 400 Hz) 12.81 (brs, 2H), 9.14 (s, 1H), 8.27 (d, 1H, J=1.96 Hz), 8.02 (s, 2H, J=8.78 Hz), 7.89 (t, 3H, J=8.29 Hz), 7.77-7.79 (m, 3H), 1.82 (s, 6H)

### Example 25

### 2-methyl-2-(4-(8-(1-methyl-1H-pyrazol-4-yl)-3H-pyrazolo[3,4-c]quinolin-1-yl)phenyl)propanenitrile (1167)

25% yield. MS (m/z) (M⁺+H): 393. ¹H-NMR: (δ, ppm, DMSO-d6, 400 Hz) 14.23 (brs, 1H), 9.20 (s, 1H), 8.13 (s, 1H), 8.08 (d, 1H, J=8.52 Hz), 7.95 (s, 1H), 7.69-8.09 (m, 5H), 3.84 (s, 3H), 1.82 (s, 6H).

### Example 26

### 2-methyl-2-(4-(8-(1-(methylsulfonyl)-1H-pyrazol-4-yl)-3H-pyrazolo[3,4-c]quinolin-1-yl)phenyl)propanenitrile (1168)

34% yield. MS (m/z) (M⁺+H): 457. ¹H-NMR: (δ, ppm, DMSO-d6, 400 Hz) 14.24 (s, 1H), 9.27 (s, 1H), 8.30 (s, 1H), 8.18 (s, 1H), 8.15 (d, 1H, J=6.88 Hz), 8.03 (d, 1H, J=8.24 Hz), 7.91 (d, 2H, J=7.98 Hz), 7.82 (d, 2H, J=7.97 Hz), 3.59 (s, 3H), 1.81 (s, 1H).

### Example 27

### 2-methyl-2-(4-(8-(1-phenyl-1H-pyrazol-4-yl)-3H-pyrazolo[3,4-c]quinolin-1-yl)phenyl)propanenitrile (1169)

30% yield. MS (m/z) (M⁺+H): 455. ¹H-NMR: (δ, ppm, DMSO-d6, 400 Hz) 14.21 (s, 1H), 9.28 (s, 1H), 8.82 (s, 1H), 8.34 (s, 1H), 8.17 (d, 1H, J=8.25 Hz), 7.99 (d, 1H, J=8.52 Hz), 7.93 (d, 4H, J=9.73 Hz), 7.85 (d, 4H, J=7.70 Hz), 7.50-7.76 (m, 2H), 7.32-7.36 (m, 1H), 1.81 (s, 6H).

### Example 28

### 2-methyl-2-(4-(8-(3-(4-phenylpiperazin-1-yl)phenyl)-3H-pyrazolo[3,4-c]quinolin-1-yl)phenyl)propanenitrile (1170)

20% yield. MS (m/z) (M⁺+H): 549. ¹H-NMR: (δ, ppm, DMSO-d6, 400 Hz) 14.26 (s, 1H), 9.32 (s, 1H), 8.34 (d, 1H), 8.23 (d, 1H, J=8.29 Hz), 7.96 (d, 3H, J=8.29 Hz), 7.79 (d, 2H, J=8.29 Hz), 7.23-7.33 (m, 4H), 7.01-7.03 (m, 4H), 6.82 (t, 1H, J=7.32 Hz), 3.36 (s, 4H), 3.34 (d, 4H, J=5.86 Hz), 1.77 (s, 6H).

### Example 29

### 2-methyl-2-(4-(8-(3-nitrophenyl)-3H-pyrazolo[3,4-c]quinolin-1-yl)phenyl)propanenitrile (1180)

63% yield. MS (m/z) (M⁺+H): 434. ¹H-NMR: (δ, ppm, DMSO-d6, 400 Hz) 14.30 (s, 1H), 9.33 (s, 1H), 8.47 (s, 1H), 8.40 (s, 1H), 8.08-8.27 (m, 4H), 7.97 (d, 2H, J=8.04 Hz), 7.73-7.83 (m, 3H), 1.78 (s, 6H).

### Example 30

### 2-methyl-2-(4-(8-(3-phenoxyphenyl)-3H-pyrazolo[3,4-c]quinolin-1-yl)phenyl)propanenitrile (1181)

30% yield. MS (m/z) (M⁺+H): 481. ¹H-NMR: (δ, ppm, DMSO-d6, 400 Hz) 9.28 (s, 1H), 8.35 (s, 1H), 8.18 (d, 1H), 7.90 (d, 3H), 7.74 (d, 2H), 7.41 (m, 4H), 7.29 (d, 1H), 7.16 (t, 1H), 7.06 (d, 2H), 6.95 (d, 1H), 1.80 (s, 6H).

### Example 31

### 2-methyl-2-(4-(8-(pyridazin-4-yl)-3H-pyrazolo[3,4-c]quinolin-1-yl)phenyl)propanenitrile (1182)

30% yield. MS (m/z) (M⁺+H): 391. ¹H-NMR: (δ, ppm, DMSO-d6, 400 Hz) 14.30 (s, 1H), 9.34 (s, 1H), 9.19 (s, 1H), 9.06 (s, 1H), 8.34-8.09 (m, 2H), 8.08 (m, 1H,), 7.94 (m, 2H), 7.84 (m, 2H), 1.79 (s, 6H).

### Example 32

### 2-methyl-2-(4-(8-(1-(phenylsulfonyl)-1H-pyrazol-4-yl)-3H-pyrazolo[3,4-c]quinolin-1-yl)phenyl)propanenitrile (1183)

24% yield. MS (m/z) (M⁺+H): 519. ¹H-NMR: (δ, ppm, DMSO-d6, 400 Hz) 14.25 (s, 1H), 9.28 (s, 1H), 8.27 (s, 1H), 8.15-8.01 (m, 5H), 7.93-7.81 (m, 5H), 7.68 (m, 2H), 1.82 (s, 6H).

### Example 33

### 2-(4-(8-(1-benzyl-1H-pyrazol-4-yl)-3H-pyrazolo[3,4-c]quinolin-1-yl)phenyl)-2-methylpropanenitrile (1184)

20% yield. MS (m/z) (M⁺+H): 469. ¹H-NMR: (δ, ppm, DMSO-d6, 400 Hz) 14.17 (s, 1H), 9.22 (s, 1H), 8.19 (d, 2H), 8.10 (d, 1H), 7.81-7.91 (m, 5H), 7.67 (s, 1H), 7.26-7.37 (m, 5H), 5.53 (s, 2H), 1.81 (s, 6H).

### Example 34

### 2-(4-(8-(1H-indol-3-yl)-3H-pyrazolo[3,4-c]quinolin-1-yl)phenyl)-2-methylpropanenitrile (1185)

23% yield. MS (m/z) (M⁺+H): 428. ¹H-NMR: (δ, ppm, DMSO-d6, 400 Hz) 11.20 (s, 1H), 9.25 (s, 1H), 8.35 (s, 1H), 8.02 (s, 1H), 7.90-7.81 (m, 5H), 7.47-7.36 (m, 3H,), 6.49 (s, 1H), 1.82 (s, 6H).

### Example 35

### 2-(4-(8-(1-isopropyl-1H-pyrazol-4-yl)-3H-pyrazolo[3,4-c]quinolin-1-yl)phenyl)-2-methylpropanenitrile (1186)

20% yield. MS (m/z) (M⁺+H): 421. ¹H-NMR: (δ, ppm, DMSO-d6, 400 Hz) 9.86 (s, 1H), 8.38 (s, 1H), 8.24 (d, 1H, J=8.8 Hz), 8.15 (d, 1H, J=2 Hz), 7.89 (m, 5H), 5.43 (m, 1H), 1.89 (s, 6H), 1.76 (d, 6H).

### Example 36

### 2-methyl-2-(4-(8-(1-(pyridin-4-ylmethyl)-1H-pyrazol-4-yl)-3H-pyrazolo[3,4-c]quinolin-1-yl)phenyl)propanenitrile (1187)

23% yield. MS (m/z) (M⁺+H): 470. ¹H-NMR: (δ, ppm, DMSO-d6, 400 Hz) 13.08 (s, 1H), 9.44 (s, 1H), 8.54 (d, 2H, J=4.61 Hz), 8.12 (m, 3H), 7.94-7.81 (m, 5H), 7.69 (s, 1H), 7.28 (d, 2H, J=6.5 Hz), 4.53 (s, 2H), 1.83 (s, 6H).

### Example 37

### 2-methyl-2-(4-(8-(pyridin-4-yl)-3H-pyrazolo[3,4-c]quinolin-1-yl)phenyl)propanenitrile (1188)

38% yield. MS (m/z) (M⁺+H): 390. ¹H-NMR: (δ, ppm, DMSO-d6, 400 Hz) 14.33 (s, 1H), 8.63 (d, 2H, J=5.36 Hz), 8.40 (s, 1H), 8.26 (d, 1H, J=8.78 Hz), 8.07 (d, 1H, J=8.77 Hz), 7.93 (d, 2H, J=7.80 Hz), 7.83 (d, 2H, J=8.29 Hz), 1.82 (s, 6H).

### Example 38

### N-(4-(1-(4-(2-cyanopropan-2-yl)phenyl)-3H-pyrazolo[3,4-c]quinolin-8-yl)phenyl)-N-methylacetamide (1189)

30% yield. MS (m/z) (M⁺+H): 460. ¹H-NMR: (δ, ppm, DMSO-d6, 400 Hz) 14.20 (s, 1H), 9.27 (s, 1H), 8.25 (s, 1H), 8.20 (d, 1H, J=8.79 Hz), 7.97 (d, 1H, J=7.81 Hz), 7.88 (d, 2H, J=8.06 Hz), 7.78 (d, 2H, J=8.05 Hz), 7.63 (d, 2H, J=7.81 Hz), 7.38 (s, 2H, J=7.81 Hz), 3.15 (s, 3H), 1.77 (s, 6H).

### Example 39

### N-(4-(1-(4-(2-cyanopropan-2-yl)phenyl)-3H-pyrazolo[3,4-c]quinolin-8-yl)phenyl)-N-methyl methanesulfonamide (1190)

30% yield. MS (m/z) (M⁺+H): 496. ¹H-NMR: (δ, ppm, DMSO-d6, 400 Hz) 14.20 (s, 1H), 9.27 (s, 1H), 8.23 (d, 1H, J=1.47 Hz), 8.20 (d, 1H, J=8.55 Hz), 7.95 (d, 1H, J=8.79 Hz), 7.88 (d, 2H, J=8.30 Hz), 7.79 (d, 2H, J=8.30 Hz), 7.61 (d, 2H, J=8.55 Hz), 7.45 (d, 2H, J=8.54 Hz), 3.26 (s, 3H), 2.93 (s, 3H), 1.78 (s, 6H).

### Example 40

### 4-(1-(4-(2-cyanopropan-2-yl)phenyl)-3H-pyrazolo[3,4-c]quinolin-8-l)benzonitrile (1191)

38% yield. MS (m/z) (M⁺+H): 414. ¹H-NMR: (δ, ppm, DMSO-d6, 400 Hz) 14.28 (s, 1H), 9.32 (s, 1H), 8.35 (d, 1H, J=1.95 Hz), 8.24 (d, 1H, J=8.19 Hz), 8.00-8.02 (m, 1H), 7.89-7.92 (m, 4H), 7.79 (d, 4H, J=8.29 Hz), 1.80 (s, 6H).

II-1b. Method 1b Aryl Halide 11 Coupling with Boronic Esters

Coupling reactions of aryl halide 11 with boronic esters were carried under three different reaction conditions (Condition a, Condition b, Condition c) and provided final compounds (31). The reaction conditions and results were summarized in the following Table, and the final compounds were summarized in Table II-1b

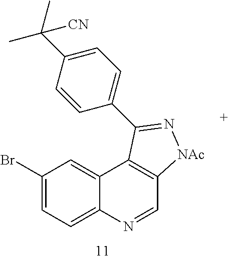

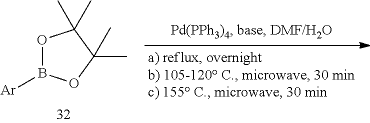

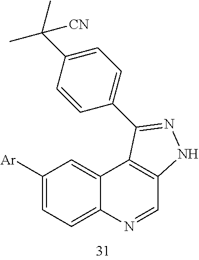

| En- | | Meth- | Yield |
|---|---|---|---|
| try | ArB(OR)₂ (32) | od | (%) |
| 1 | 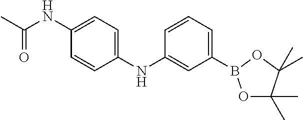 | b | 10 2 steps |
| 2 | 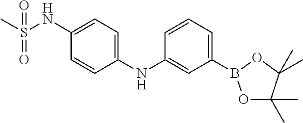 | b | 11 2 steps |
| 3 | 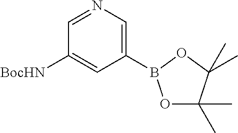 | b | 38 |
| 4 | 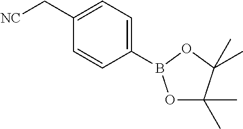 | b 1 hr | 38 |
| 5 | 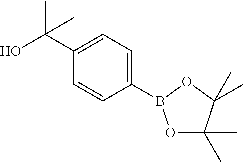 | b 1 hr | 34 |
| 6 | 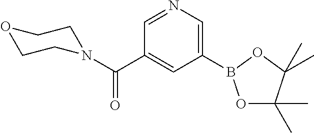 | b | 23 |
| 7 | 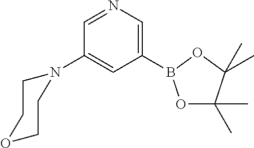 | b | 21 |
| 8 | 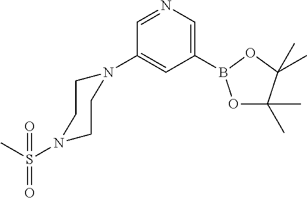 | b | 22 |
| 9 | 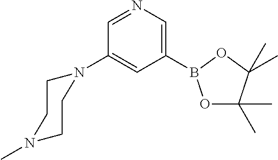 | b | 80 |
| 10 | 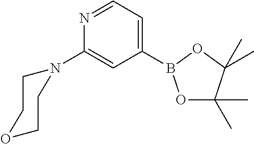 | c | 40 |
| 11 | 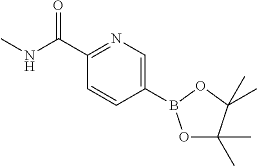 | b | 22 |
| 12 | 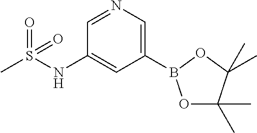 | b | 46 |
| 13 | 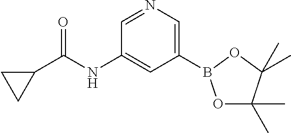 | b | 22 |
| 14 | 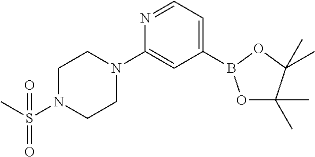 | c | 50 |
| 15 | 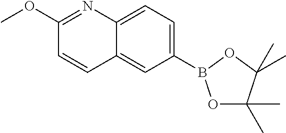 | c | 45 |
| 16 | 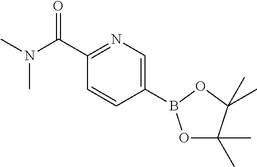 | b | 20 |
| 17 | 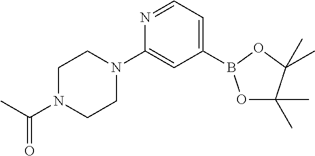 | a or c | 50 |
| 18 | 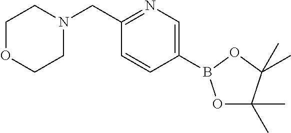 | a | 20% |
| 19 | 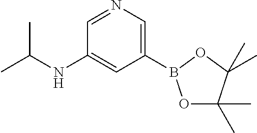 | a | 30% |
| 20 | 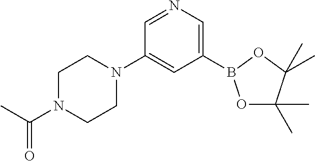 | c | 60 |
| 21 | 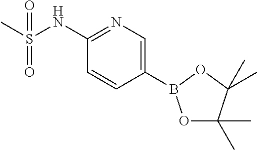 | b | 35 |
| 22 | 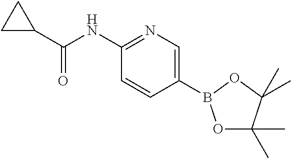 | b | 27 |
| 23 | 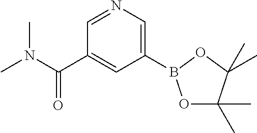 | b | 24 |
| 24 | 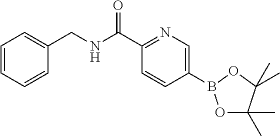 | a | 40 |
| 25 | 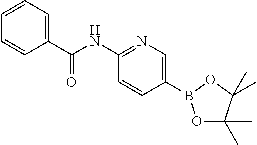 | a or c | 27 |
| 26 | 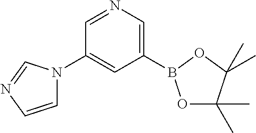 | b | 30 |
| 27 | 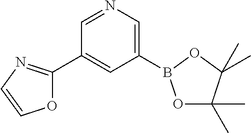 | b | 21 |
| 28 | 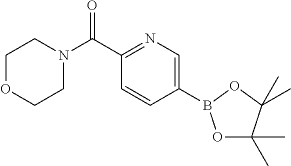 | a | 38 |
| 29 | 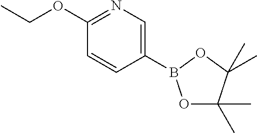 | a | 32 |
| 30 | 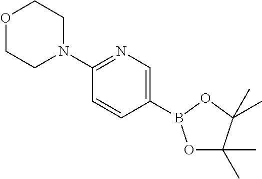 | a | 30 |
| 31 | 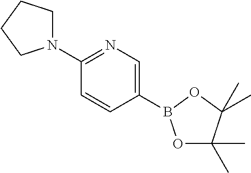 | a | 20 |
| 32 | 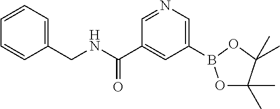 | b | 23 |
| 33 | 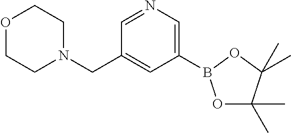 | a | 31 |
| 34 | 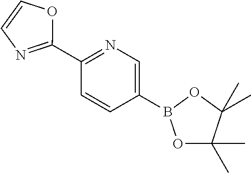 | a | 22 |
| 35 | 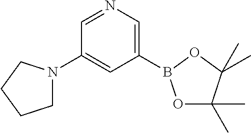 | b | 23 |
| 36 | 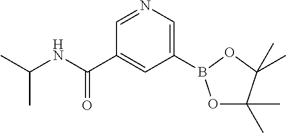 | a | 20 |
| 37 | 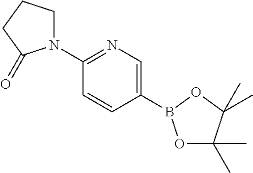 | a | 29 |
| 38 | 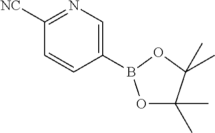 | a | 38 |
| 39 | 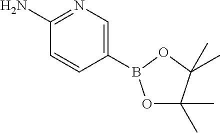 | a | 29 |
| 40 | 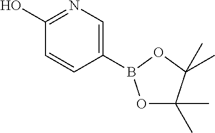 | a | 22 |
| 41 | 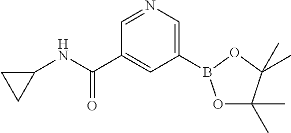 | a | 29 |

TABLE II-1b

| Cpd | | | | MS | |
|---|---|---|---|---|---|
| No. | Ex | Structure | MF/MW | (M⁺ + H) | IUPAC |
| 1192 | 1 | 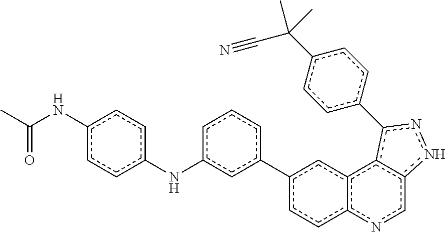 | C34H28N6O/ 536.6 | 537 | N-(4-(3-(1-(4-(2- cyanopropan-2-yl)phenyl)- 3H-pyrazolo[3,4-c]quinolin- 8-yl)phenylamino) phenyl)acetamide |
| 1193 | 2 | 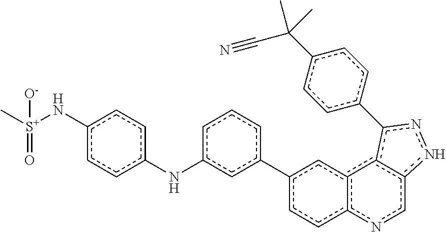 | C33H28N6O2S/ 572.7 | 573 | N-(4-(3-(1-(4-(2- cyanopropan-2-yl)phenyl)- 3H-pyrazolo[3,4-c] quinolin-8-yl)phenylamino) phenyl)methanesulfonamide |
| 1194 | 3 | 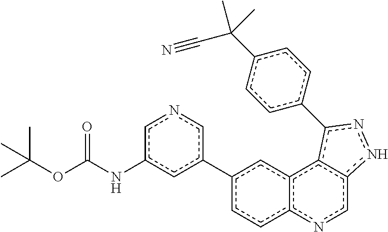 | C30H28N6O2/ 504.6 | 505 | tert-butyl 5-(1-(4-(2- cyanopropan-2-yl)phenyl)- 3H-pyrazolo[3,4-c]quinolin- 8-yl)pyridin-3-ylcarbamate |
| 1195 | 4 | 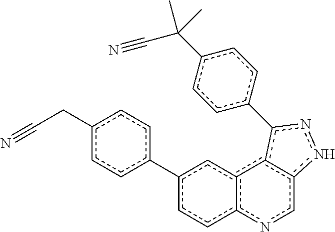 | C28H21N5/ 427.5 | 428 | 2-(4-(8-(4-(cyanomethyl) phenyl)-3H-pyrazolo [3,4-c]quinolin-1-yl)phenyl)- 2-methylpropanenitrile |
| 1196 | 5 | 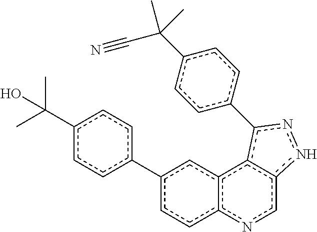 | C29H26N4O/ 446.5 | 447 | 2-(4-(8-(4-(2- hydroxypropan-2-yl)phenyl)- 3H-pyrazolo[3,4-c] quinolin-1-yl)phenyl)- 2-methylpropanenitrile |
| 1197 | 6 | 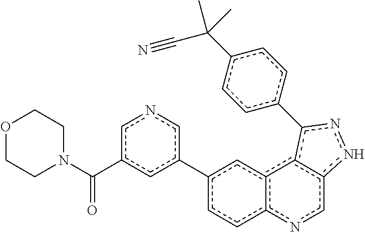 | C30H26N6O2/ 502.6 | 525 | 2-methyl-2-(4-(8-(5- (morpholine-4- carbonyl)pyridin-3-yl)-3H- pyrazolo[3,4-c]quinolin-1- yl)phenyl)propanenitrile |
| 1198 | 7 | 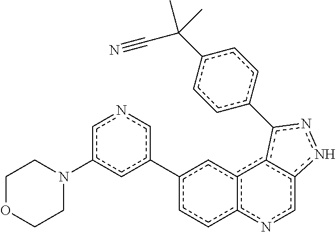 | C29H26N6O/ 474.6 | 475 | 2-methyl-2-(4-(8-(5- morpholinopyridin-3-yl)-3H- pyrazolo[3,4-c]quinolin-1- yl)phenyl)propanenitrile |
| 1199 | 8 | 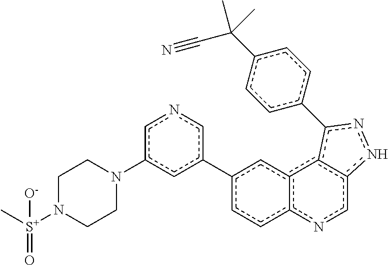 | C30H29N7O2S/ 551.7 | 552 | 2-methyl-2-(4-(8- (5-(4-(methylsulfonyl) piperazin-1-yl)pyridin- 3-yl)-3H-pyrazolo[3,4-c] quinolin-1-yl)phenyl) propanenitrile |
| 1200 | 9 | 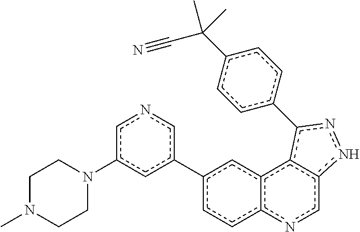 | C30H29N7/ 487.6 | 488 | 2-methyl-2-(4-(8- (5-(4-methylpiperazin- 1-yl)pyridin-3-yl)-3H- pyrazolo[3,4-c]quinolin-1- yl)phenyl)propanenitrile |
| 1201 | 10 | 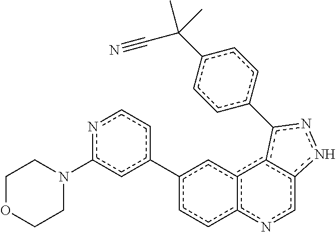 | C29H26N6O/ 474.6 | 475 | 2-methyl-2-(4-(8-(2- morpholinopyridin-4-yl)-3H- pyrazolo[3,4-c]quinolin-1- yl)phenyl)propanenitrile |
| 1202 | 11 | 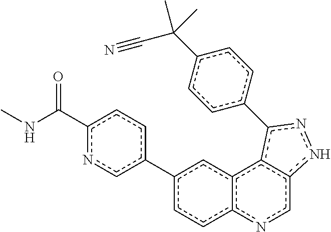 | C27H22N6O/ 446.5 | 447 | 5-(1-(4-(2-cyanopropan- 2-yl)phenyl)-3H-pyrazolo [3,4-c]quinolin-8-yl)-N- methylpicolinamide |
| 1203 | 12 | 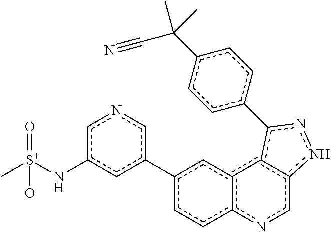 | C26H22N6O2S/ 482.6 | 483 | N-(5-(1-(4-(2-cyanopropan- 2-yl)phenyl)-3H-pyrazolo [3,4-c]quinolin-8-yl) pyridin-3-yl) methanesulfonamide |
| 1204 | 13 | 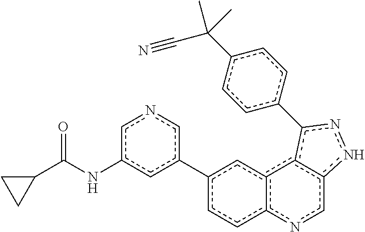 | C33H30N6O3/ 472.5 | 472 | N-(5-(1-(4-(2-cyanopropan- 2-yl)phenyl)-3H-pyrazolo [3,4-c]quinolin-8-yl)pyridin- 3-yl)cyclopropane- carboxamide cyclopropanecarboxylate salt |
| 1205 | 14 | 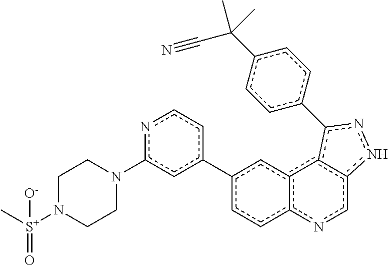 | C30H29N7O2S/ 551.7 | 550 | 2-methyl-2-(4-(8- (2-(4-(methylsulfonyl) piperazin-1-yl)pyridin-4-yl)- 3H-pyrazolo[3,4-c] quinolin-1-yl)phenyl) propanenitrile |
| 1206 | 15 | 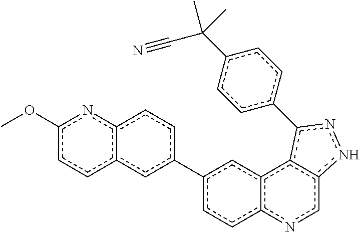 | C30H23N5O/ 469.5 | 470 | 2-(4-(8-(2-methoxyquinolin- 6-yl)-3H-pyrazolo[3,4-c] quinolin-1-yl)phenyl)- 2-methylpropanenitrile |
| 1207 | 16 | 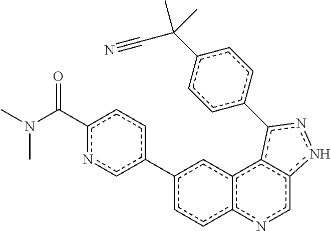 | C28H24N6O/ 460.5 | 461 | 5-(1-(4-(2-cyanopropan-2-yl) phenyl)-3H-pyrazolo [3,4-c]quinolin-8-yl)- N,N-dimethylpicolinamide |
| 1208 | 17 | 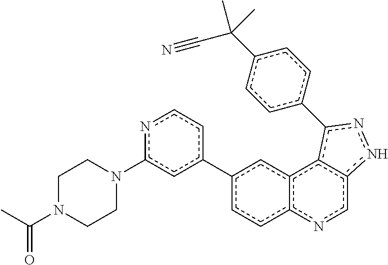 | C31H29N7O/ 515.6 | 516 | 2-(4-(8-(2-(4-acetyl- piperazin-1-yl)pyridin-4- yl)-3H-pyrazolo[3,4- c]quinolin-1-yl)phenyl)-2- methylpropanenitrile |
| 1209 | 18 | 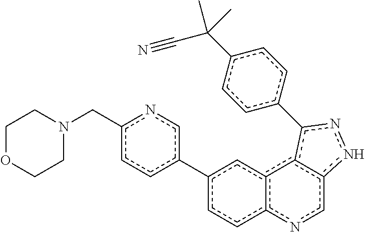 | C30H28N6O/ 488.6 | 489 | 2-methyl-2-(4-(8-(6- (morpholinomethyl)pyridin- 3-yl)-3H-pyrazolo[3,4-c] quinolin-1-yl)phenyl) propanenitrile |
| 1210 | 19 | 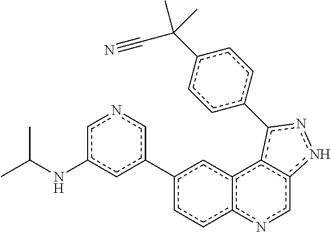 | C28H26N6/ 446.5 | 447 | 2-(4-(8-(5- (isopropylamino)pyridin- 3-yl)-3H-pyrazolo[3,4-c] quinolin-1-yl)phenyl)-2- methylpropanenitrile |
| 12011 | 20 | 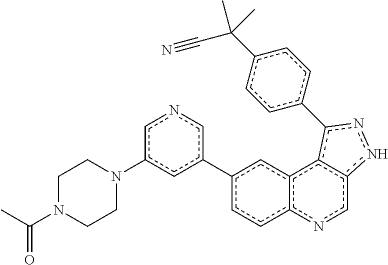 | C31H29N7O/ 515.6 | 516 | 2-(4-(8-(5-(4-acetyl- piperazin-1-yl)pyridin- 3-yl)-3H-pyrazolo [3,4-c]quinolin-1- yl)phenyl)-2- methylpropanenitrile |
| 1212 | 21 | 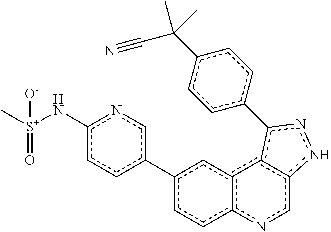 | C26H22N6O2S/ 482.6 | 483 | N-(5-(1-(4-(2- cyanopropan-2-yl)phenyl)- 3H-pyrazolo[3,4-c] quinolin-8-yl)pyridin-2- yl)methanesulfonamide |
| 1213 | 22 | 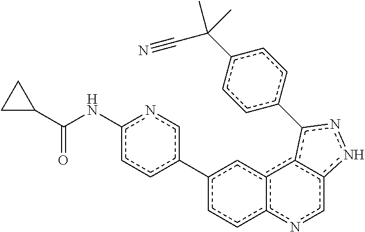 | C33H30N6O3/ 472.5 | 473 | N-(5-(1-(4-(2-cyanopropan- 2-yl)phenyl)-3H-pyrazolo [3,4-c]quinolin-8-yl)pyridin- 2-yl)cyclopropane- carboxamide cyclopropanecarboxylate |
| 1214 | 23 | 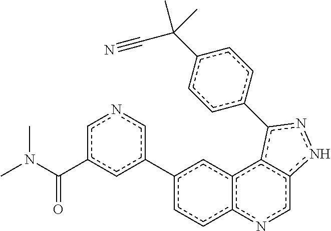 | C28H24N6O/ 460.5 | 461; M − 1 459 | 5-(1-(4-(2-cyanopropan-2- yl)phenyl)-3H-pyrazolo [3,4-c]quinolin-8-yl)-N,N- dimethylnicotinamide |
| 1215 | 24 | 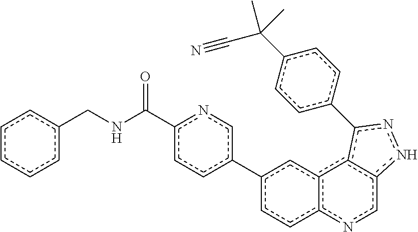 | C33H26N6O/ 522.6 | M − 1 521 | N-benzyl-5-(1-(4-(2- cyanopropan-2-yl)phenyl)- 3H-pyrazolo[3,4-c]quinolin- 8-yl)picolinamide |
| 1216 | 25 | 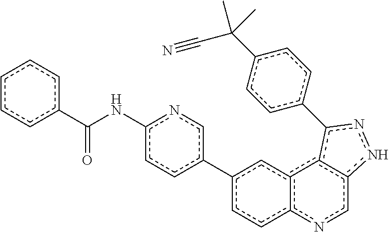 | C32H24N6O/ 508.6 | 509. | N-(5-(1-(4-(2-cyanopropan- 2-yl)phenyl)-3H-pyrazolo [3,4-c]quinolin-8-yl)pyridin- 2-yl)benzamide |
| 1217 | 26 | 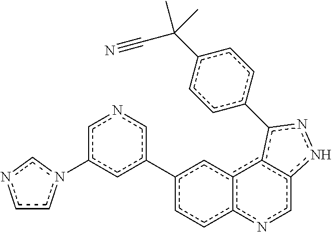 | C28H21N7/ 455.5 | 456. | 2-(4-(8-(5-(1H-imidazol- 1-yl)pyridin-3-yl)-3H- pyrazolo[3,4-c]quinolin- 1-yl)phenyl)-2- methylpropanenitrile |
| 1218 | 27 | 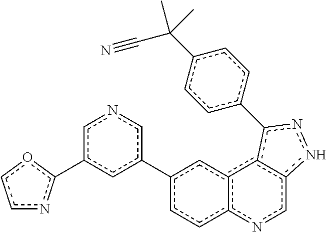 | C28H20N6O/ 456.5 | 457. | 2-methyl-2-(4-(8-(5- (oxazol-2-yl)pyridin-3-yl)- 3H-pyrazolo[3,4-c]quinolin- 1-yl)phenyl)propanenitrile |
| 1219 | 28 | 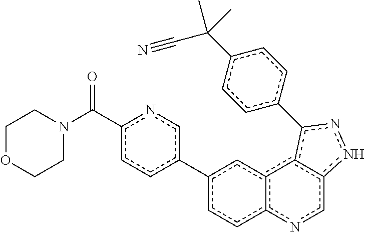 | C30H26N6O2/ 502.6 | 503 | 2-methyl-2-(4-(8-(6- (morpholine-4- carbonyl)pyridin-3-yl)-3H- pyrazolo[3,4-c]quinolin-1- yl)phenyl)propanenitrile |
| 1220 | 29 | 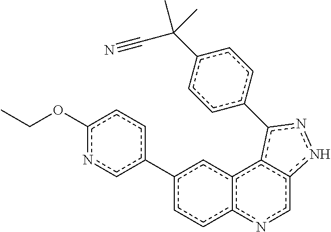 | C27H23N5O/ 433.5 | 434 | 2-(4-(8-(6-ethoxypyridin- 3-yl)-3H-pyrazolo[3,4-c] quinolin-1-yl)phenyl)-2- methylpropanenitrile |
| 1221 | 30 | 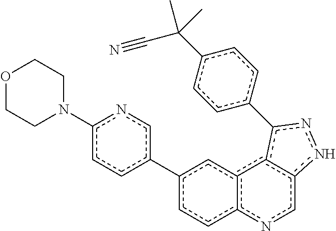 | C29H26N6O/ 474.6 | 475 | 2-methyl-2-(4-(8-(6- morpholinopyridin-3-yl)- 3H-pyrazolo[3,4-c] quinolin-1-yl)phenyl) propanenitrile |
| 1222 | 31 | 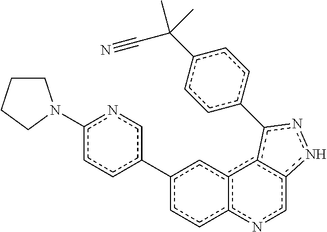 | C29H26N6/ 458.6 | 459 | 2-methyl-2-(4-(8-(6- (pyrrolidin-1-yl)pyridin- 3-yl)-3H-pyrazolo[3,4-c] quinolin-1-yl) phenyl)propanenitrile |
| 1223 | 32 | 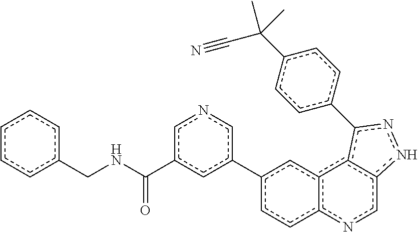 | C33H26N6O/ 522.6 | M − 1 521 | N-benzyl-5-(1-(4-(2- cyanopropan-2-yl)phenyl)- 3H-pyrazolo[3,4-c] quinolin-8-yl)nicotinamide |
| 1224 | 33 | 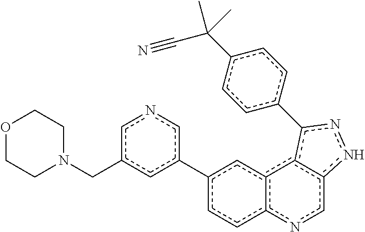 | C30H28N6O/ 488.6 | 489 | 2-methyl-2-(4-(8-(5- (morpholinomethyl) pyridin-3-yl)-3H-pyrazolo [3,4-c]quinolin-1- yl)phenyl)propanenitrile |
| 1225 | 34 | 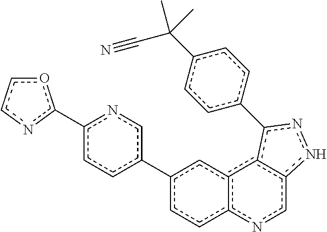 | C28H20N6O/ 456.5 | 457 | 2-methyl-2-(4-(8-(6- (oxazol-2-yl)pyridin-3-yl)- 3H-pyrazolo[3,4-c] quinolin-1-yl)phenyl) propanenitrile |
| 1226 | 35 | 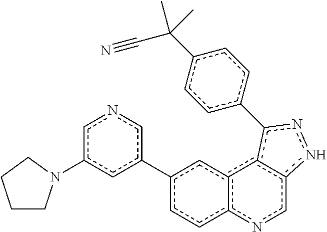 | C29H26N6/ 458.6 | 459 | 2-methyl-2-(4-(8-(5- (pyrrolidin-1-yl)pyridin- 3-yl)-3H-pyrazolo[3,4-c] quinolin-1-yl)phenyl) propanenitrile |
| 1227 | 36 | 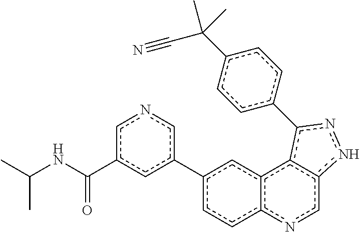 | C29H26N6O/ 474.6 | 475 | 5-(1-(4-(2-cyanopropan-2- yl)phenyl)-3H-pyrazolo [3,4-c]quinolin-8-yl)-N- isopropylnicotinamide |
| 1228 | 37 | 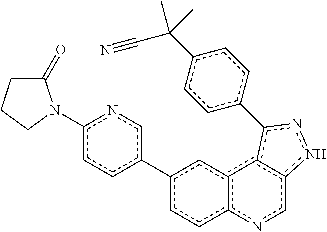 | C29H24N6O/ 472.5 | 473 | 2-methyl-2-(4-(8-(6-(2- oxopyrrolidin-1-yl)pyridin- 3-yl)-3H-pyrazolo[3,4- c]quinolin-1-yl) phenyl)propanenitrile |
| 1229 | 38 | 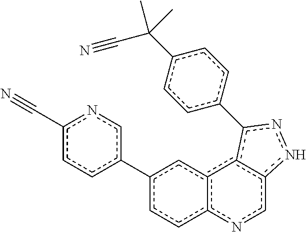 | C26H18N6/ 414.5 | M + Na4 37 | 5-(1-(4-(2-cyanopropan-2- yl)phenyl)-3H-pyrazolo [3,4-c]quinolin-8-yl) picolinonitrile |
| 1230 | 39 | 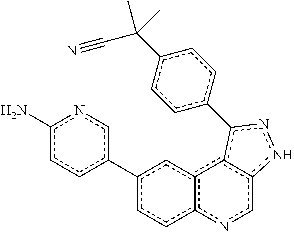 | C25H20N6/ 404.5 | 405 | 2-(4-(8-(6-aminopyridin- 3-yl)-3H-pyrazolo[3,4-c] quinolin-1-yl)phenyl)-2- methylpropanenitrile |
| 1231 | 40 | 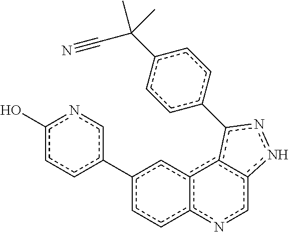 | C25H19N5O/ 405.5 | 406 | 2-(4-(8-(6-hydroxypyridin- 3-yl)-3H-pyrazolo[3,4- c]quinolin-1-yl)phenyl)-2- methylpropanenitrile |
| 1232 | 41 | 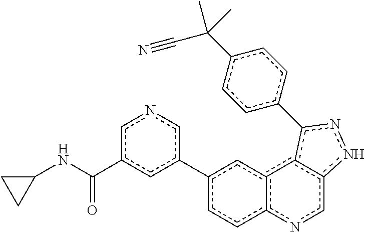 | C29H24N6O/ 472.5 | 472. | 5-(1-(4-(2-cyanopropan- 2-yl)phenyl)-3H-pyrazolo [3,4-c]quinolin-8-yl)-N- cyclopropylnicotinamide |

Synthetic Procedures for Preparing Compounds in Table II-1b.

### General Syntheses Procedure for Compounds in Table II-1b: (Example 3)

To a solution of 2-(4-(3-acetyl-8-bromo-3H-pyrazolo[3,4-c]quinolin-1-yl)phenyl)-2-methylpropanenitrile (58 mg, 0.14 mmol) in DMF (2 mL) was added tert-butyl 5-(4,4,5,5-tetramethyl-1,3,2-dioxaborolan-2-yl)pyridin-3-ylcarbamate (50 mg, 0.16 mmol), 2 M K₂CO₃ aqueous solution (0.42 mmol, 0.21 mL) and Pd(PPh₃)₄ (10 mg). The reaction mixture was stirred under microwave for 30 min at 155° C. The mixture was diluted with water (10 mL) and extracted with DCM (3×20 mL). Organic layer was washed with brine, dried over Na₂SO₄, filtered, and the filtrate was concentrated. The residue was purified by column chromatography to give product.

### Example 1

### N-(4-(3-(1-(4-(2-cyanopropan-2-yl)phenyl)-3H-pyrazolo[3,4-c]quinolin-8-yl)phenylamino)phenyl)acetamide (1192)

10% yield for 2 steps. MS (m/z) (M⁺+H): 537. ¹H NMR (δ, ppm, DMSO-d6, 400 Hz) 9.67 (s, 1H), 8.46 (d, 1H, J=1.96 Hz), 8.38 (d, 1H, J=7.46 Hz), 8.09-8.15 (m, 3H), 7.95 (d, 2H, J=4.7 Hz), 7.88 (d, 2H, J=4.69 Hz), 7.61-7.73 (m, 5H), 7.45-7.48 (m, 1H), 1.99 (s, 3H), 1.81 (s, 6H).

### Example 2

### N-(4-(3-(1-(4-(2-cyanopropan-2-yl)phenyl)-3H-pyrazolo[3,4-c]quinolin-8-yl)phenylamino)phenyl)methanesulfonamide (1193)

11% for 2 steps. MS (m/z) (M⁺+H): 573. ¹H-NMR: (δ, ppm, MeOH-D4, 400 Hz) 9.99 (s, 1H), 8.51-8.56 (m, 2H), 8.31 (d, 1H, J=10.56 Hz), 8.23 (d, 2H, J=9.00 Hz), 7.78-8.05 (m, 7H), 7.58 (d, 2H, J=7.05 Hz), 7.50-7.52 (m, 5H), 2.70 (s, 3H), 1.86 (s, 6H).

### Example 3

### tert-butyl 5-(1-(4-(2-cyanopropan-2-yl)phenyl)-3H-pyrazolo[3,4-c]quinolin-8-yl)pyridin-3-yl carbamate (1194)

60% yield. MS (m/z) (M⁺+H): 505.3. ¹H-NMR: (δ, ppm, MeOH-D4, 400 Hz) 9.24 (s, 1H), 8.56 (d, 1H, J=2.20 Hz), 8.19-8.35 (m, 4H), 7.80-7.90 (m, 5H), 1.82 (s, 6H), 1.57 (s, 9H).

### Example 4

### 2-(4-(8-(4-(cyanomethyl)phenyl)-3H-pyrazolo[3,4-c]quinolin-1-yl)phenyl)-2-methylpropanenitrile (1195)

38% yield. MS (m/z) (M⁺+H): 428. ¹H-NMR: (δ, ppm, DMSO-d6, 400 Hz) 9.28 (s, 1H), 8.19-8.29 (m, 2H), 7.79-7.97 (m, 5H), 7.63 (d, 2H, J=8.72 Hz), 7.42 (d, 2H, J=8.22 Hz), 4.08 (s, 2H), 1.80 (s, 6H).

### Example 5

### 2-(4-(8-(4-(2-hydroxypropan-2-yl)phenyl)-3H-pyrazolo[3,4-c]quinolin-1-yl)phenyl)-2-methylpropanenitrile (1196)

34% yield. MS (m/z) (M⁺+H): 447. ¹H-NMR: (δ, ppm, MeOH-D4, 400 Hz) 9.19 (s, 1H), 8.30 (s, 1H), 8.19 (d, 1H, J=8.60 Hz), 7.95 (d, 1H, J=4.65 Hz), 7.81-7.88 (m, 3H), 7.61-7.67 (m, 111), 7.51-7.58 (m, 4H), 1.85 (s, 6H), 1.55 (s, 6H).

### Example 6

### 2-methyl-2-(4-(8-(5-(morpholine-4-carbonyl)pyridin-3-yl)-3H-pyrazolo[3,4-c]quinolin-1-yl)phenyl)propanenitrile (1197)

23% yield. MS (m/z) (M⁺+H): 525. ¹H-NMR: (δ, ppm, DMSO-d6, 400 Hz) 9.93 (s, 1H), 9.24 (d, 1H, J=1.96 Hz), 8.93 (d, 1H, J=5.68 Hz), 8.86 (d, 1H, J=6.26 Hz), 8.66 (s, 1H), 8.60 (d, 1H, J=8.51 Hz), 8.35-8.32 (m, 2H), 8.20-8.23 (m, 1H), 8.00-8.02 (m, 2H), 7.89-7.91 (m, 2H), 3.87-3.90 (m, 4H), 3.21-3.24 (m, 4H), 1.85 (s, 6H).

### Example 7

### 2-methyl-2-(4-(8-(5-morpholinopyridin-3-yl)-3H-pyrazolo[3,4-c]quinolin-1-yl)phenyl)propanenitrile (1198)

21% yield. MS (m/z) (M⁺+H): 475. ¹H-NMR: (δ, ppm, MeOH-D4, 400 Hz) 10.03 (s, 1H), 8.52-8.56 (m, 2H), 8.24-8.26 (m, 2H,), 8.14 (t, 1H, J=2.35 Hz), 8.00-8.03 (m, 1H), 7.93-7.97 (m, 5H), 3.89-3.91 (m, 4H), 3.22-3.24 (m, 4H), 1.85 (s, 6H).

### Example 8

### 2-methyl-2-(4-(8-(5-(4-(methylsulfonyl)piperazin-1-yl)pyridin-3-yl)-3H-pyrazolo[3,4-c]quinolin-1-yl)phenyl)propanenitrile (1199)

22% yield MS (m/z) (M⁺+H): 552. ¹H-NMR: (δ, ppm, MeOH-D4, 400 Hz) 10.04 (s, 1H), 8.56 (d, 1H, J=1.76 Hz), 8.51 (d, 1H, J=8.81 Hz), 8.25-8.27 (m, 2H), 8.12 (d, 1H, J=1.17 Hz), 8.00-8.02 (m, 2H), 7.92-7.95 (m, 2H), 3.52-3.55 (m, 4H), 3.31-3.35 (m, 4H), 2.96 (s, 3H), 1.85 (s, 6H).

### Example 9

### 2-methyl-2-(4-(8-(5-(4-methylpiperazin-1-yl)pyridin-3-yl)-3H-pyrazolo[3,4-c]quinolin-1-yl)phenyl)propanenitrile (1200)

80% yield. MS (m/z) (M⁺+H): 488. ¹H-NMR: (δ, ppm, DMSO-d6, 400 Hz) 9.31 (s, 1H), 8.32 (m, 2H), 8.21 (m, 2H), 7.99 (m, 3H), 7.95 (d, 2H), 7.49 (m, 1H), 3.31 (s, 8H), 2.22 (s, 3H), 1.76 (s, 6H).

### Example 10

### 2-methyl-2-(4-(8-(2-morpholinopyridin-4-yl)-3H-pyrazolo[3,4-c]quinolin-1-yl)phenyl)propanenitrile (1201)

40% yield. MS (m/z) (M⁺+H): 475. ¹H-NMR: (δ, ppm, DMSO-d6, 400 Hz) 14.29 (s, 1H), 9.32 (s, 1H), 8.39 (s, 1H), 8.22 (m, 2H), 7.96 (m, 3H), 7.78 (d, 2H), 7.05 (s, 1H), 6.85 (d, 1H), 3.72 (s, 4H), 3.50 (s, 4H), 1.72 (s, 6H).

### Example 11

### 5-(1-(4-(2-cyanopropan-2-yl)phenyl)-3H-pyrazolo[3,4-c]quinolin-8-yl)-N-methylpicolinamide (1202)

22% yield MS (m/z) (M⁺+H): 447. ¹H-NMR: (δ, ppm, DMSO-d6, 400 Hz) 9.12 (s, 1H), 8.37 (s, 1H), 8.04-8.02 (d, 1H), 7.88-7.86(m, 4H), 7.69-7.61 (m, 3H), 7.43 (s, 1H), 3.41 (s, 3H), 1.75 (s, 6H).

### Example 12

### N-(5-(1-(4-(2-cyanopropan-2-yl)phenyl)-3H-pyrazolo[3,4-c]quinolin-8-yl)pyridin-3-yl)methanesulfonamide (1203)

46% yield. MS (m/z) (M⁺+H): 483. ¹H-NMR: (δ, ppm, MeOH-D4, 400 Hz) 10.06 (s, 1H), 8.98 (s, 1H), 8.78 (s, 1H), 8.69 (d, 2H), 8.58-8.56 (d, 1H), 8.37-8.39 (d, 1H), 8.04-8.02 (d, 21-I), 7.94-7.92 (d, 2H), 3.30 (s, 3H), 1.85 (s, 6H).

### Example 13

### N-(5-(1-(4-(2-cyanopropan-2-yl)phenyl)-3H-pyrazolo[3,4-c]quinolin-8-yl)pyridin-3-yl) cyclopropanecarboxamide cyclopropanecarboxylate (1204)

22% yield. MS (m/z) (M⁺+H): 472 as free base. ¹H-NMR: (δ, ppm, MeOH-D4, 400 Hz) 10.06 (s, 1H), 9.31 (s, 1H), 9.02 (s, 1H), 8.90 (s, 1H), 8.67 (s, 1H), 8.55 (d, 1H, J=8.52 Hz), 8.33 (d, 1H, J=8.24 Hz), 8.04-7.95 (m, 4H), 3.22-3.19 (m, 1H), 2.02-2.00 (m, 1H), 1.85 (s, 6H), 1.33-1.28 (m, 2H), 1.09-1.01 (m, 2H).

### Example 14

### 2-methyl-2-(4-(8-(2-(4-(methylsulfonyl)piperazin-1-yl)pyridin-4-yl)-3H-pyrazolo[3,4-c]quinolin-1-yl)phenyl)propanenitrile (1205)

50% yield. MS (m/z) (M⁺+H): 550. ¹H-NMR: (δ, ppm, DMSO-d6, 400 Hz) 14.29 (s, 1H), 9.32 (s, 1H), 8.38 (s, 1H), 8.23 (m, 2H), 8.04 (m, 3H), 7.93 (m, 2H), 7.13 (s, 1H), 6.82 (m, 1H), 3.69 (t, 4H), 3.22 (t, 4H), 2.89 (s, 3H), 1.78 (s, 6H).

### Example 15

### 2-(4-(8-(2-methoxyquinolin-6-yl)-3H-pyrazolo[3,4-c]quinolin-1-yl)phenyl)-2-methylpropanenitrile (1206)

45% yield. MS (m/z) (M⁺+H): 470. ¹H-NMR: (δ, ppm, DMSO-d6, 400 Hz) 9.28 (s, 1H), 8.46 (s, 1H), 8.05 (m, 4H), 7.91 (m, 5H), 7.03 (d, 1H), 3.99 (s, 3H), 1.78 (s, 6H).

### Example 16

### 5-(1-(4-(2-cyanopropan-2-yl)phenyl)-3H-pyrazolo[3,4-c]quinolin-8-yl)-N,N-dimethylpicolinamide (1207)

20% yield. MS (m/z) (M⁺+H): 461. ¹H-NMR: (δ, ppm, MeOH-D4, 400 Hz) 10.05 (s, 1H), 8.57-8.51 (m, 2H), 8.27-8.24 (m, 2H), 8.13-8.12 (m, 1H), 8.06-7.93 (m, 5H), 3.04 (s, 3H), 2.90 (s, 3H), 1.85 (s, 6H).

### Example 17

### 2-(4-(8-(2-(4-acetylpiperazin-1-yl)pyridin-4-yl)-3H-pyrazolo[3,4-c]quinolin-1-yl)phenyl)-2-methylpropanenitrile (1208)

50% yield. MS (m/z) (M⁺+H): 516. ¹H-NMR: (δ, ppm, DMSO-d6, 400 Hz) 14.29 (s, 1H), 9.32 (s, 1H), 8.39 (s, 1H), 8.14 (m, 2H), 8.03 (m, 3H), 7.79 (m, 2H), 7.07 (s, 1H), 6.83 (d, 1H), 3.61 (d, 8H), 2.04 (s, 3H), 1.78 (s, 6H).

### Example 18

### 2-methyl-2-(4-(8-(6-(morpholinomethyl)pyridin-3-yl)-3H-pyrazolo[3,4-c]quinolin-1-yl)phenyl)propanenitrile (1209)

20% yield. MS (m/z) (M⁺+H): 489. ¹H-NMR: (δ, ppm, DMCO-d6, 400 MHz): 9.98 (s, 1H), 8.75 (s, 1H), 8.37-8.34 (d, 1H), 8.27 (s, 1H), 8.15-8.13 (d, 1H), 8.04-7.94 (m, 5H), 7.61-7.59 (d, 1H), 3.67-3.64 (m, 6H), 2.49-2.47 (t, 4H), 1.90 (s, 6H). (δ, ppm, CDCl3, 400 MHz): 9.30 (s, 1H), 8.81 (s, 1H), 8.38-8.33 (d, 2H), 7.91-7.88 (m, 3H), 7.83-7.81 (d, 1H), 7.74-7.72 (d, 2H), 7.50-7.48 (d, 1H), 3.78-3.71 (m, 6H), 2.55 (s, 4H), 1.84 (s, 6H).

### Example 19

### 2-(4-(8-(5-(isopropylamino)pyridin-3-yl)-3H-pyrazolo[3,4-c]quinolin-1-yl)phenyl)-2-methylpropanenitrile (1210

30% yield. MS (m/z) (M⁺+H): 447. ¹H-NMR: (δ, ppm, DMCO-d6, 400 MHz): 9.33 (s, 1H), 8.44 (s, 1H), 8.24 (d, 1H), 8.06 (s, 1H), 8.01 (m, 3H), 7.94 (m, 1H), 7.84 (d, 2H), 7.14 (s, 1H), 3.75 (m, 1H), 1.86 (s, 6H), 1.26 (d, 6H).

### Example 20

### 2-(4-(8-(5-(4-acetylpiperazin-1-yl)pyridin-3-yl)-3H-pyrazolo[3,4-c]quinolin-1-yl)phenyl)-2-methylpropanenitrile (1211)

60% yield. MS (m/z) (M⁺+H): 516. ¹H-NMR: (δ, ppm, DMSO-d6, 300 Hz) 9.35 (s, 1H), 8.37 (s, 2H), 8.27 (d, 2H), 8.02 (m, 3H), 7.82 (m, 2H), 7.56 (s, 1H), 3.67 (m, 4H), 3.29 (m, 4H), 2.10 (s, 3H), 1.81 (s, 6H).

### Example 21

### N-(5-(1-(4-(2-cyanopropan-2-yl)phenyl)-3H-pyrazolo[3,4-c]quinolin-8-yl)pyridin-2-yl)methanesulfonamide (1212)

35% yield. MS (m/z) (M⁺+H): 483. ¹H-NMR: (δ, ppm, MeOH-D4, 400 Hz) 10.00 (s, 1H), 8.60 (d, 1H, J=8.81 Hz), 8.41 (s, 1H), 8.38 (s, 1H), 8.29 (d, 1H, J=1.95 Hz), 8.13-7.80 (m, 5H), 7.21 (d, 1H, J=9.59 Hz), 3.84 (s, 3H), 1.89 (s, 6H).

### Example 22

### N-(5-(1-(4-(2-cyanopropan-2-yl)phenyl)-3H-pyrazolo[3,4-c]quinolin-8-yl)pyridin-2-yl) cyclopropanecarboxamide cyclopropanecarboxylate (1213)

27% yield. MS (m/z) (M⁺+H): 473 as free base. ¹H-NMR: (δ, ppm, MeOH-D4, 400 Hz) 10.03 (s, 1H), 8.68-8.64 (m, 2H), 8.58 (s, 1H), 8.54 (d, 1H, J=8.61 Hz), 8.31 (d, 1H, J=9.97 Hz), 8.02 (d, 1H, J=8.02 Hz), 7.92 (d, 2H, J=8.02 Hz), 7.70 (d, 2H, J=8.81 Hz), 3.15-3.13 (m, 1H), 2.04-2.01 (m, 1H), 1.86 (s, 6H), 1.28-1.25 (m, 4H).

### Example 23

### 5-(1-(4-(2-cyanopropan-2-yl)phenyl)-3H-pyrazolo[3,4-c]quinolin-8-yl)-N,N-dimethylnicotinamide (1214)

11 mg, 24% yield. MS (m/z) (M⁺+H): M-1459. H¹ NMR (δ, ppm, DMSO-d₆, 400 MHz): 14.37 (s, 1H), 9.33 (s, 1H), 8.78 (s, 1H), 8.61 (s, 1H), 8.81 (s, 1H), 8.23-8.09 (m, 3H), 7.87-7.7.77 (m, 5H), 2.99 (s, 3H), 2.92 (s, 3H), 1.80 (s, 6H).

### Example 24

### N-benzyl-5-(1-(4-(2-cyanopropan-2-yl)phenyl)-3H-pyrazolo[3,4-c]quinolin-8-yl)picolinamide (1215)

40% yield. MS (m/z) (M⁺+H): 521. ¹H-NMR: (δ, ppm, CDCl3, 400 MHz): 11.20 (s, 1H), 8.71 (s, 1H), 8.39-8.27 (m, 4H), 8.01-7.87 (m, 4H), 7.74-7.72 (d, 2H), 7.38-7.35 (m, 4H), 4.71 (d, 2H), 1.82 (s, 6H); (δ, ppm, MeOH-D4, 400 MHz): 9.28 (s, 1H), 8.85 (s, 1H), 8.42 (s, 1H), 8.30 (d, 1H), 8.17 (d, 1H), 8.06 (d, 1H), 7.92-7.84 (m, 4H), 7.39-7.27 (m, 5H), 4.64 (s, 2H), 1.83 (s, 6H).

### Example 25

### N-(5-(1-(4-(2-cyanopropan-2-yl)phenyl)-3H-pyrazolo[3,4-c]quinolin-8-yl)pyridin-2-yl)benzamide (1216)

13 mg, 27% yield. MS (m/z) (M⁺+H): 509 ¹H-NMR: (δ, ppm, MeOH-d4, 400 MHz): 10.08 (s, 1H), 9.50 (s, 1H), 9.40 (s, 1H), 9.33 (s, 1H), 8.85 (s, 1H), 8.55 (d, 1H, J=8.80 Hz), 8.46 (d, 1H, J=8.80 Hz), 8.31 (s, 1H), 8.10 (d, 2H, J=8.80 Hz), 7.96 (d, 2H, J=7.60 Hz), 7.61 (s, 1H), 1.86 (s, 6H).

### Example 26

### 2-(4-(8-(5-(1H-imidazol-1-yl)pyridin-3-yl)-3H-pyrazolo[3,4-c]quinolin-1-yl)phenyl)-2-methylpropanenitrile (1217)

22 mg, 30% yield. MS (m/z) (M⁺+H): 456. ¹H-NMR: (δ, ppm, MeOH-d4, 400 MHz): 10.04 (s, 1H), 9.78 (s, 1H), 9.23 (s, 1H), 9.16 (s, 1H), 8.85 (s, 1H), 8.72 (s, 1H), 8.52 (d, 1H, J=8.80 Hz), 8.45 (d, 1H, J=8.80 Hz), 8.32 (s, 1H), 7.91 (d, 2H, J=8.22 Hz), 7.88-7.86 (m, 3H), 1.81 (s, 6H).

### Example 27

### 2-methyl-2-(4-(8-(5-(oxazol-2-yl)pyridin-3-yl)-3H-pyrazolo[3,4-c]quinolin-1-yl)phenyl)propanenitrile (1218)

18 mg, 21% yield. MS (m/z) (M⁺+H): 457. ¹H-NMR: (δ, ppm, MeOH-d4, 400 MHz): 10.08 (s, 1H), 9.78 (s, 1H), 9.23 (s, 1H), 9.16 (s, 1H), 8.85 (s, 1H), 8.72 (s, 1H), 8.52 (d, 1H, J=8.80 Hz), 8.45 (d, 1H, J=8.80 Hz), 8.32 (s, 1H), 7.91 (d, 2H, J=8.22 Hz), 7.88-7.86 (m, 3H), 1.81 (s, 6H).

### Example 28

### 2-methyl-2-(4-(8-(6-(morpholine-4-carbonyl)pyridin-3-yl)-3H-pyrazolo[3,4-c]quinolin-1-yl)phenyl)propanenitrile (1219)

38% yield. MS (m/z) (M⁺+H): 503. ¹H-NMR: (δ, ppm, acetone-d6, 400 Hz) 9.37 (s, 1H), 8.85 (d, 1H), 8.49 (d, 1H), 8.19 (d, 1H), 8.07 (m, 1H), 7.99 (m, 5H), 7.74 (d, 1H), 3.73 (s, 4H), 3.66 (d, 4H), 1.87 (s, 6H).

### Example 29

### 2-(4-(8-(6-ethoxypyridin-3-yl)-3H-pyrazolo[3,4-c]quinolin-1-yl)phenyl)-2-methylpropanenitrile (1220)

Yield: 32%. MS (m/z) (M⁺+H): 434. ¹H-NMR: (δ, ppm, acetone-d6, 400 Hz) 9.27 (s, 1H), 8.40 (s, 1H), 8.17 (s, 1H), 7.95-7.78 (m, 6H), 6.86-6.84 (d, 1H, J=8.8 Hz), 4.34-4.29 (q, 2H), 1.79 (s, 6H), 1.33-1.29 (t, 3H).

### Example 30

### 2-methyl-2-(4-(8-(6-morpholinopyridin-3-yl)-3H-pyrazolo[3,4-c]quinolin-1-yl)phenyl)propanenitrile (1221)

Yield: 30%. MS (m/z) (M⁺+H): 475. ¹H-NMR: (δ, ppm, acetone-d6, 400 Hz) 9.28 (s, 1H), 8.45 (m, 1H), 8.21 (m, 1H), 7.97 (d, 1H), 7.92 (m, 6H), 6.89 (d, 1H), 3.74 (m, 4H), 3.53 (s, 4H), 1.87 (s, 6H).

### Example 31

### 2-methyl-2-(4-(8-(6-(pyrrolidin-1-yl)pyridin-3-yl)-3H-pyrazolo[3,4-c]quinolin-1-yl)phenyl)propanenitrile(1222)

Yield: 20%. MS (m/z) (M⁺+H): 459. ¹H-NMR: (δ, ppm, DMCO-d6, 400 MHz): 9.26 (s, 1H), 8.40 (s, 1H), 8.26 (s, 1H), 8.20-8.18 (d, 1H), 7.97-7.86 (m, 5H), 7.74-7.71 (dd, 1H), 6.51 (d, 1H), 3.48-4.45 (s, 4H), 1.89 (s, 6H).

### Example 32

### N-benzyl-5-(1-(4-(2-cyanopropan-2-yl)phenyl)-3H-pyrazolo[3,4-c]quinolin-8-yl)nicotinamide (1223)

Yield: 12 mg, 23%. MS (m/z) (M⁺+H): 521. ¹H-NMR (δ, ppm, MeOH-d4, 400 MHz): 10.04 (s, 1H), 9.46 (s, 1H), 9.41 (s, 1H), 9.36 (s, 1H), 8.81 (s, 1H), 8.49 (d, 1H, J=8.40 Hz), 8.05 (d, 2H, J=8.40 Hz), 7.91 (d, 2H, J=8.00 Hz), 7.43 (d, 2H, J=6.80 Hz), 7.38 (m, 2H), 7.28-7.23 (m, 1H), 4.67 (s, 2H), 1.78 (s, 6H).

### Example 33

### 2-methyl-2-(4-(8-(5-(morpholinomethyl)pyridin-3-yl)-3H-pyrazolo[3,4-c]quinolin-1-yl)phenyl)propanenitrile (1224)

Yield: 31%. MS (m/z) (M⁺+H): 489. ¹H-NMR: (δ, ppm, CDCl3, 400 MHz): 9.28 (s, 1H), 8.71 (s, 1H), 8.56 (s, 1H), 8.44 (s, 1H), 8.34 (d, 2H), 7.89 (m, 4H), 7.73 (d, 2H), 3.73 (t, 4H), 3.59 (s, 2H), 2.50 (s, 4H), 1.83 (s, 6H).

### Example 34

### 2-methyl-2-(4-(8-(6-(oxazol-2-yl)pyridin-3-yl)-3H-pyrazolo[3,4-c]quinolin-1-yl)phenyl)propanenitrile (1225)

Yield: 10 mg, 22% as a light yellow solid. MS (m/z) (M⁺+H): 457. ¹H-NMR (δ, ppm, MeOH-d4, 400 MHz): 10.06 (s, 1H), 9.29(s, 1H), 8.97 (d, 1H, J=5.20 Hz), 8.92 (d, 1H, J=8.40 Hz), 8.69 (d, 1H, J=1.60 Hz), 8.55 (d, 1H, J=13.60 Hz), 8.40-8.38 (m, 1H), 8.29-8.25 (m, 1H), 8.09 (s, 1H), 8.03 (d, 2H, J=8.40 Hz), 7.91 (d, 2H, J=8.00 Hz), 1.85 (s, 6H).

### Example 35

### 2-methyl-2-(4-(8-(5-(pyrrolidin-1-yl)pyridin-3-yl)-3H-pyrazolo[3,4-c]quinolin-1-yl)phenyl)propanenitrile (1226)

Yield: 10 mg, 23%. MS (m/z) (M⁺+H): 459. ¹H-NMR (δ, ppm, MeOH-d4, 400 MHz): 10.04 (s, 1H), 9.30 (s, 1H), 8.99-8.93 (m, 1H), 8.67 (d, 1H, J=2.00 Hz), 8.58 (d, 1H, J=11.60 Hz), 8.42-8.39 (m, 1H), 8.31-8.26 (m, 1H), 8.03 (d, 2H, J=11.20 Hz), 7.91 (d, 2H, J=8.80 Hz), 3.29 (m, 4H), 2.03-1.99 (m, 4H), 1.85 (s, 6H).

### Example 36

### 5-(1-(4-(2-cyanopropan-2-yl)phenyl)-3H-pyrazolo[3,4-c]quinolin-8-yl)-N-isopropylnicotinamide (1227)

Yield: 18 mg, 38%. MS (m/z) (M⁺+H): 475. ¹H-NMR (δ, ppm, MeOH-d₄, 400 MHz): 9.88 (s, 1H), 9.13 (d, 2H, J=8.80 Hz), 8.83-8.67 (m, 2H), 8.42 (d, 1H, J=8.40 Hz), 8.31(d, 1H, J=8.80 Hz), 7.99 (d, 2H, J=8.00 Hz), 7.90 (d, 2H, J=8.00 Hz), 4.30-4.26 (m, 1H), 1.85 (s, 6H), 1.31 (d, 6H, J=6.80 Hz).

### Example 37

### 2-methyl-2-(4-(8-(6-(2-oxopyrrolidin-1-yl)pyridin-3-yl)-3H-pyrazolo[3,4-c]quinolin-1-yl)phenyl)propanenitrile (1228)

35 mg, yield: 29%. ¹H-NMR (400 MHz, CDCl₃) δ: 9.28 (s, 1H), 8.54 (d, 1H, J=2.0 Hz), 8.46 (d, 1H, J=8.8 Hz), 8.33 (s, 1H), 8.31 (s, 1H), 7.84-7.88 (m, 4H), 7.74 (s, 1H), 7.72 (s, 1H), 4.14 (t, 2H, J=14.4 Hz), 2.70 (t, 2H, J=16.4 Hz), 2.15-2.19 (m, 2H), 1.85 (s, 6H).

### Example 38

### 5-(1-(4-(2-cyanopropan-2-yl)phenyl)-3H-pyrazolo[3,4-c]quinolin-8-yl)picolinonitrile (1229)

Yield: 18 mg, 38%. MS (m/z) (M⁺+Na): 437. ¹H-NMR (δ, ppm, DMSO-d₆, 400 MHz): 14.33 (s, 1H), 9.36 (s, 1H), 9.02 (d, 1H, J=2.00 Hz), 8.40 (s, 1H), 8.30-8.25 (m, 2H), 8.14-8.10 (m, 2H), 7.94 (d, 2H, J=8.40 Hz), 7.79 (d, 2H, J=8.00 Hz), 1.79 (s, 6H).

### Example 39

### 2-(4-(8-(6-aminopyridin-3-yl)-3H-pyrazolo[3,4-c]quinolin-1-yl)phenyl)-2-methylpropanenitrile (1230)

Yield: 30 mg, 29%. MS (m/z) (M⁺+Na): 437. ¹H-NMR (δ, ppm, MeOH-d4, 400 MHz): 9.18 (s, 1H), 8.24(s, 1H), 8.14-8.18(m, 2H), 7.82-7.91(m, 5H), 7.67(d, 1H), 6.64(d, 1H), 1.86(s, 6H).

### Example 40

### 2-(4-(8-(6-hydroxypyridin-3-yl)-3H-pyrazolo[3,4-c]quinolin-1-yl)phenyl)-2-methylpropanenitrile (1231)

Yield: 22%. MS (m/z) (M⁺+Na): 437. ¹H-NMR (δ, ppm, MeOH-D4, 400 MHz): 9.22 (s, 1H), 8.21 (s, 1H), 8.18 (s, 1H), 7.71-7.91 (m, 8H), 7.71 (s, 1H), 6.63 (d, 1H), 1.86 (s, 6H).

### Example 41

### 5-(1-(4-(2-cyanopropan-2-yl)phenyl)-3H-pyrazolo[3,4-c]quinolin-8-yl)-N-cyclopropylnicotinamide (1232)

14 mg, 30%. MS (m/z) (M⁺+H): 472. ¹H-NMR (δ, ppm, DMSO-d₆, 400 MHz): 9.66 (s, 1H), 9.07-9.05 (m, 2H), 8.91 (d, 1H, J=4.40 Hz), 8.58 (d, 1H, J=2.00 Hz), 8.48 (d, 1H, J=4.40 Hz), 8.39 (d, 1H, J=8.40 Hz), 8.24 (d, 1H, J=8.80 Hz), 7.90 (d, 2H, J=8.40 Hz), 7.81 (d, 2H, J=8.40 Hz), 2.94-2.89 (m, 1H), 1.79 (s, 6H), 0.78-0.73 (m, 2H), 0.64-0.61 (m, 2H).

II-2. Method 2 Aryl Borate 33 Coupling with Aryl Halides

Coupling reaction of aryl halide 11 with diboron provided boronic ester 33, which was coupled with aryl halides under three different reaction conditions (Condition a, Condition b, Condition c) and provided final compounds (31). The reaction conditions and results were summarized in the following Table, and the final compounds were summarized in Table II-2.

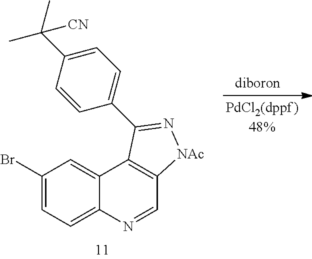

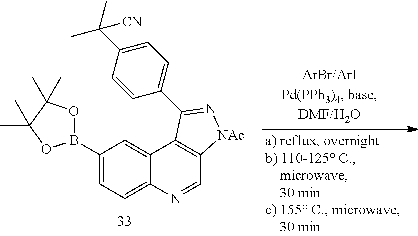

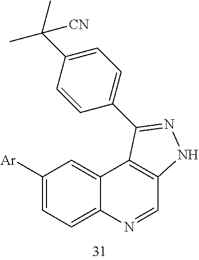

| | | | Yield |
|---|---|---|---|
| Entry | ArBr/ArI | Method | (%) |
| 1 | 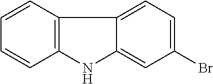 | c | 21 |
| 2 | 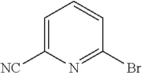 | c | 26 |
| 3 | 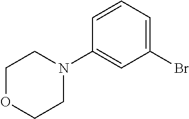 | c | 25 |
| 4 | 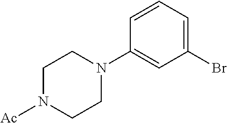 | c | 20 |
| 5 | 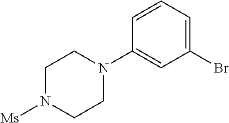 | c | 18 |
| 6 | 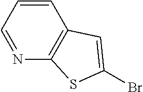 | c | 22 |
| 7 | 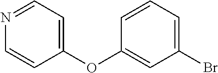 | c | 16 |
| 8 | 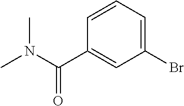 | c | 39 |
| 9 | 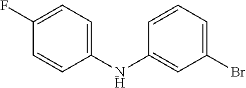 | c | 20 |
| 10 | 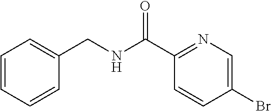 | c | 40% 2 steps |
| 11 | 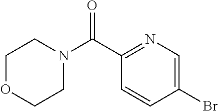 | c | 38% |

Note:
Examples 10 and 11 were also prepared by method a in II-1a.

TABLE II-2

| Cpd | | | | MS | |
|---|---|---|---|---|---|
| No | Ex | Structure | MF/MW | (M⁺ + H) | IUPAC |
| 1233 | 1 | 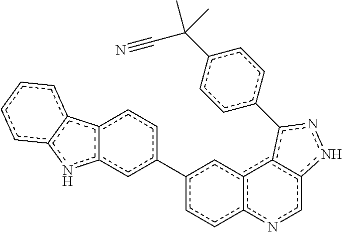 | C32H23N5/ 477.6 | 478 | 2-(4-(8-(9H-carbazol-2-yl)- 3H-pyrazolo[3,4-c]quinolin-1- yl)phenyl)-2- methylpropanenitrile |
| 1234 | 2 | 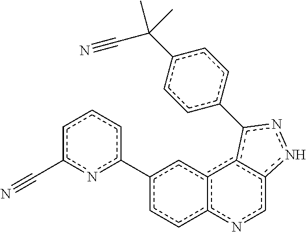 | C26H18N6/ 414.5 | 415 | 6-(1-(4-(2-cyanopropan-2- yl)phenyl)-3H-pyrazolo[3,4- c]quinolin-8-yl)picolinonitrile |
| 1235 | 3 | 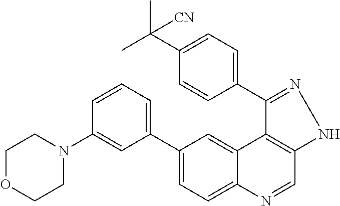 | C30H27N5O/ 473.22 | 474 | 2-methyl-2-(4-(8-(3- morpholinophenyl)-3H- pyrazolo[3,4-c]quinolin-1- yl)phenyl)propanenitrile |
| 1236 | 4 | 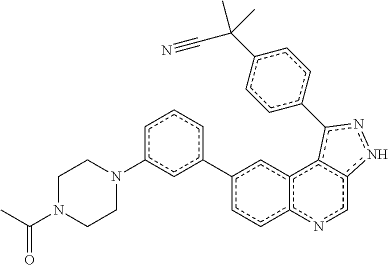 | C32H30N6O/ 514.6 | 515 | 2-(4-(8-(3-(4-acetylpiperazin- 1-yl)phenyl)-3H-pyrazolo[3,4- c]quinolin-1-yl)phenyl)-2- methylpropanenitrile |
| 1237 | 5 | 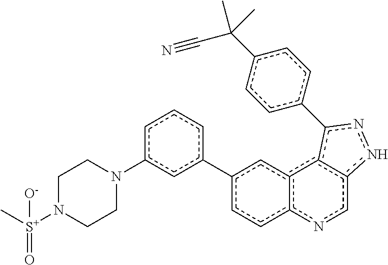 | C31H30N6O2S/ 550.7 | 593 | 2-methyl-2-(4-(8-(3-(4- (methylsulfonyl)piperazin-1- yl)phenyl)-3H-pyrazolo[3,4- c]quinolin-1- yl)phenyl)propanenitrile |
| 1238 | 6 | 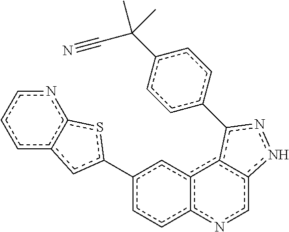 | C27H19N5S/ 445.5 | 446 | 2-methyl-2-(4-(8-(thieno [2,3-b]pyridin-2-yl)-3H- pyrazolo[3,4-c]quinolin-1- yl)phenyl)propanenitrile |
| 1239 | 7 | 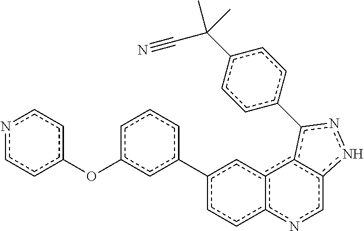 | C31H23N5O/ 481.5 | 482 | 2-methyl-2-(4-(8-(3-(pyridin- 4-yloxy)phenyl)-3H- pyrazolo[3,4-c]quinolin-1- yl)phenyl)propanenitrile |
| 1240 | 8 | 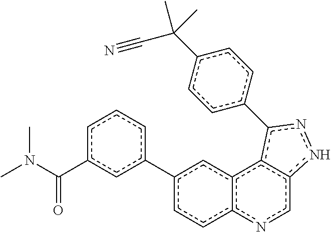 | C29H25N5O/ 459.5 | 460 | 3-(1-(4-(2-cyanopropan-2- yl)phenyl)-3H-pyrazolo[3,4- c]quinolin-8-yl)-N,N- dimethylbenzamide |
| 1241 | 9 | 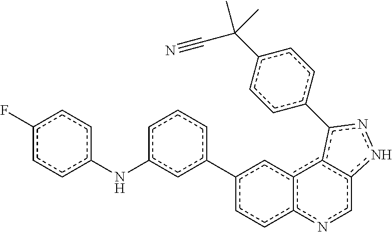 | C32H24FN5/ 497.6 | 498 | 2-(4-(8-(3-(4- fluorophenylamino)phenyl)- 3H-pyrazolo[3,4-c]quinolin-1- yl)phenyl)-2- methylpropanenitrile |
| 1242 | 10 | 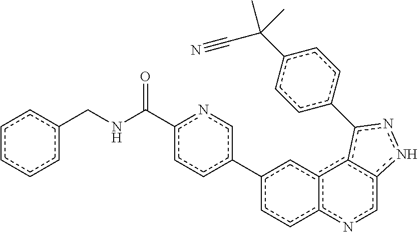 | C33H26N6O/ 522.6 | M − 1 521 | N-benzyl-5-(1-(4-(2- cyanopropan-2-yl)phenyl)-3H- pyrazolo[3,4-c]quinolin-8- yl)picolinamide |
| 1243 | 11 | 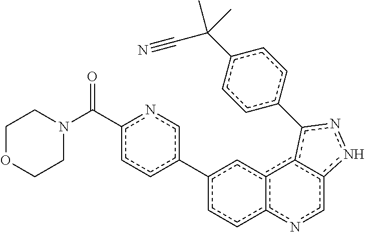 | C30H26N6O2/ 502.6 | 503 | 2-methyl-2-(4-(8-(6- (morpholine-4- carbonyl)pyridin-3-yl)-3H- pyrazolo[3,4-c]quinolin-1- yl)phenyl)propanenitrile |

Synthetic Procedures for Preparing Compounds in Table II-2.

### 2-(4-(3-acetyl-8-(4,4,5,5-tetramethyl-1,3,2-dioxaborolan-2-yl)-3H-pyrazolo[3,4-c]quinolin-1-yl)phenyl)-2-methylpropanenitrile (33)

A mixture of 11 (1 g, 0.0023 mmol), bis(pinacolato)diboron (0.7 g, 0.003 mmol), PdCl₂(dppf)₂ (0.085 g, 0.001 mmol) and KOAc (1.2 g, 0.0115 mmol) in toluene (20 mL) was stirred under N₂ at 110° C. for 12 h. The solution was diluted with H₂O, extracted with EA. The organic layer was washed with brine, dried over Na₂SO₄ and purified by column chromatography with (silica gel, EA/PE) to give product (0.64 g, 65%). MS (m/z) (M⁺+H): 481 (as ester), 399 (as acid). ¹H-NMR: (δ, ppm, CDCl₃, 400 Hz): 10.11 (s, 1H), 8.65 (s, 1H), 8.26 (d, 1H), 8.07 (dd, 1H), 7.89 (t, 2H), 7.75 (t, 2H), 2.91 (s, 3H), 1.86 (s, 6H), 1.32 (s, 12H).

### General Syntheses Procedure for Compounds in Table II-2 (Example 1)

To a solution of 33 (50 mg, 0.1 mmol) in DMF (2.5 mL) was added 2-bromo-9H-carbazole (29 mg, 0.12 mmol), 2M Na₂CO₃(0.15 mmol, 0.15 mL) and Pd(PPh₃)₄ (10 mg). The reaction mixture was stirred under microwave for 30 min at 155° C. The mixture was diluted with water (10 mL) and extracted with DCM (3×20 mL). Organic layer was washed with brine, dried over Na₂SO₄, filtered, and the filtrate was concentrated. The residue was purified by silica gel column chromatography (DCM:Methanol 80:1 to 60:1) to give the desired product (10 mg, 21%) as a light yellow solid. MS (m/z) (M⁺+H): 478. ¹H-NMR (δ, ppm, DMSO-d6, 400 MHz): 11.40 (s, 1H), 9.27 (s, 1H), 8.45 (s, 1H), 8.20 (m, 2H), 7.94 (m, 3H), 7.80 (m, 2H), 7.72 (m, 1H), 7.48 (m, 1H), 7.40 (m, 2H), 7.17 (m, 1H), 1.73 (s, 6H).

### Example 2

### 6-(1-(4-(2-cyanopropan-2-yl)phenyl)-3H-pyrazolo[3,4-c]quinolin-8-yl)picolinonitrile (1234)

26% yield. MS (m/z) (M⁺+H): 415. ¹H-NMR: (δ, ppm, DMSO-d6, 400 Hz) 9.34 (s, 1H), 9.08 (s, 1H), 8.30 (m, 3H), 8.13 (t, 1H), 7.98 (m, 3H), 7.79 (d, 2H, J=8.22 Hz), 1.81 (s, 6H).

### Example 3

### 2-methyl-2-(4-(8-(3-morpholinophenyl)-3H-pyrazolo[3,4-c]quinolin-1-yl)phenyl)propanenitrile (1235)

25% yield. MS (m/z) (M⁺+H): 474. ¹H-NMR: (δ, ppm, DMSO-d6, 400 Hz) 9.30 (s, 1H), 8.40 (s, 1H), 8.32 (d, 1H, J=8.61 Hz), 7.84 (m, 3H), 7.73 (m, 3H), 7.34 (t, 1H), 7.12 (s, 1H), 7.05 (d, 1H, J=7.24 Hz), 6.93 (m, 1H), 3.90 (t, 4H), 3.17 (t, 4H), 1.82 (s, 6H).

### Example 4

### 2-(4-(8-(3-(4-acetylpiperazin-1-yl)phenyl)-3H-pyrazolo[3,4-c]quinolin-1-yl)phenyl)-2-methylpropanenitrile (1236)

20% yield. MS (m/z) (M⁺+H): 515. ¹H-NMR: (δ, ppm, DMSO-d6, 400 Hz) 9.28 (s, 1H), 8.30 (s, 1H), 8.20 (m, 1H), 7.94 (d, 3H, J=8.22 Hz), 7.77 (d, 2H, J=8.21 Hz), 7.30 (t, 1H), 7.17 (s, 1H), 6.99 (t, 2H), 3.62 (3, 4H), 3.14 (m, 4H), 2.04 (s, 3H), 1.76 (s, 6H).

### Example 5

### 2-methyl-2-(4-(8-(3-(4-(methylsulfonyl)piperazin-1-yl)phenyl)-3H-pyrazolo[3,4-c]quinolin-1-yl)phenyl)propanenitrile (1237)

18% yield MS (m/z) (M⁺+H): 593. ¹H-NMR: (δ, ppm, DMSO-d6, 400 Hz) 9.17 (s, 1H), 8.35 (s, 1H), 8.09 (d, 1H, J=8.61 Hz), 7.93 (d, 2H, J=8.22 Hz), 7.80 (d, 1H, J=8.21 Hz), 7.71 (d, 2H, J=8.61 Hz), 7.29 (t, 1H), 7.21 (s, 1H), 7.05 (m, 2H), 2.92 (s, 3H), 1.76 (s, 6H).

### Example 6

### 2-methyl-2-(4-(8-(thieno[2,3-b]pyridin-2-yl)-3H-pyrazolo[3,4-c]quinolin-1-yl)phenyl)propanenitrile (1238)

22% yield MS (m/z) (M⁺+H): 446. ¹H-NMR: (δ, ppm, DMSO-d6, 400 Hz) 9.31 (s, 1H), 8.53 (m, 1H), 8.33 (s, 1H), 8.18 (m, 3H), 7.90 (m, 5H), 7.44 (m, 1H), 1.85 (s, 6H).

### Example 7

### 2-methyl-2-(4-(8-(3-(pyridin-4-yloxy)phenyl)-3H-pyrazolo[3,4-c]quinolin-1-yl)phenyl)propanenitrile (1239)

16% yield. MS (m/z) (M⁺+H): 482. ¹H-NMR: (δ, ppm, DMSO-d6, 400 Hz) 9.31 (s, 1H), 8.48 (m, 2H), 8.38 (d, 1H, J=2.2 Hz), 8.24 (d, 1H, J=8.80 Hz), 8.04 (m, 1H), 7.94 (d, 2H, J=8.54 Hz), 7.77 (d, 2H, J=8.24 Hz), 7.57 (m, 2H), 7.44 (s, 1H), 7.19 (m, 1H), 6.97 (m, 2H), 1.72 (s, 6H).

### Example 8

### 3-(1-(4-(2-cyanopropan-2-yl)phenyl)-3H-pyrazolo[3,4-c]quinolin-8-yl)-N,N-dimethylbenzamide (1240)

39% yield. MS (m/z) (M⁺+H): 460. ¹H-NMR: (δ, ppm, DMSO-d6, 400 Hz) 14.21 (s, 1H), 9.29 (s, 1H), 8.38 (s, 1H), 8.23 (d, 2H, J=8.54 Hz), 8.00 (d, 2H, J=8.05 Hz), 7.93 (d, 2H, J=8.30 Hz), 7.77 (m, 3H), 7.62 (s, 1H), 7.52 (m, 1H), 7.40 (d, 1H, J=7.81 Hz), 3.01 (d, 6H, J=35.64 Hz), 1.79 (s, 6H).

### Example 9

### 2-(4-(8-(3-(4-fluorophenylamino)phenyl)-3H-pyrazolo[3,4-c]quinolin-1-yl)phenyl)-2-methylpropanenitrile (1241)

20% yield. MS (m/z) (M⁺+H): 498. ¹H-NMR: (δ, ppm, DMSO-d6, 400 Hz) 14.25 (s, 1H), 9.28 (s, 1H), 8.28 (m, 2H), 8.19 (m, 1H), 7.89 (m, 3H), 7.75 (m, 2H), 7.22 (m, 2H), 7.08 (m, 3H), 7.00 (m, 2H), 1.69 (s, 6H).

### Example 10

### N-benzyl-5-(1-(4-(2-cyanopropan-2-yl)phenyl)-3H-pyrazolo[3,4-c]quinolin-8-yl)picolinamide (1242)

40% yield. MS (m/z) (M⁺+H): 521. ¹H-NMR: (δ, ppm, CDCl3, 400 MHz): 11.20 (s, 1H), 9.30 (s, 1H), 8.71 (s, 1H), 8.39-8.27 (m, 4H), 8.01-7.87 (m, 4H), 7.74-7.72 (d, 2H), 7.38-7.35 (m, 4H), 4.71 (d, 2H), 1.82 (s, 6H); (δ, ppm, MeOH-D4, 400 MHz): 9.28 (s, 1H), 8.85 (s, 1H), 8.42 (s, 1H), 8.30 (d, 1H), 8.17 (d, 1H), 8.06 (d, 1H), 7.92-7.84 (m, 4H), 7.39-7.27 (m, 5H), 4.64 (s, 2H), 1.83 (s, 6H).

### Example 11

### 2-methyl-2-(4-(8-(6-(morpholine-4-carbonyl)pyridin-3-yl)-3H-pyrazolo[3,4-c]quinolin-1-yl)phenyl)propanenitrile (1243)

38% yield. MS (m/z) (M⁺+H): 503. ¹H-NMR: (δ, ppm, DMCO-d6, 400 Hz) 9.37 (s, 1H), 8.85 (d, 1H), 8.49 (d, 1H), 8.19 (d, 1H), 8.07 (m, 1H), 7.99 (m, 5H), 7.74 (d, 1H), 3.73 (s, 4H), 3.66 (d, 4H), 1.87 (s, 6H).

II-3. Method 3. Aryl Borate 34 Coupling with Aryl Halides Followed by Ring Closure

Coupling reaction of aryl halide 23 with diboron provided boronic ester 34, which was under Suzuki coupling reaction with aryl halides to provide 35 (a to J). Reduction of nitro compounds, followed by ring closure, afforded the final compounds 37 (a to J). All reaction yields were summarized in the following Table, and the final compounds were summarized in Table II-3.

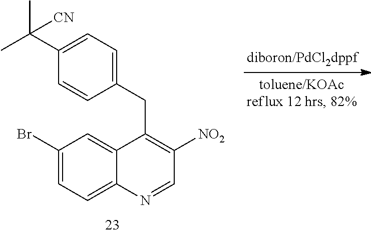

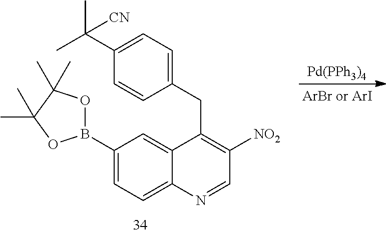

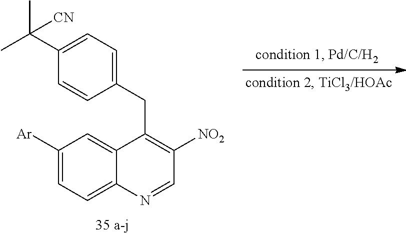

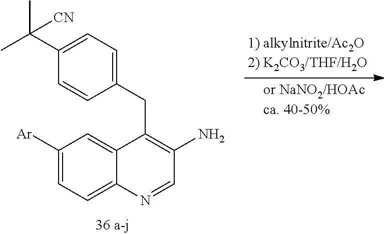

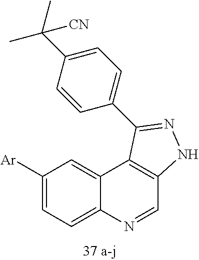

| Entry | ArBr/ArI | Yield(%)¹ |
|---|---|---|
| 1 | 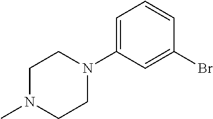 | 73%² 73%³ 22%⁴ |
| 2 | 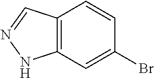 | 44%² 82%³ 34*80%⁴ |
| 3 | 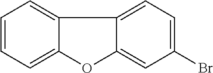 | 60%² 97%³ 27*80%⁴ |
| 4 | 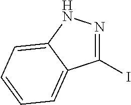 | 33%² 86%³ 50*80% |
| 5 | 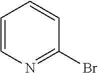 | 83%² 100%³ 64%⁴ |
| 6 | 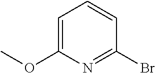 | 52%² 96%³ 20*90%⁴ |
| 7 | 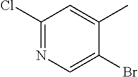 | 37%² 80%³ 44%⁴ |
| 8 | 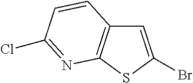 | 82.5%² 88%³ 29*19%⁴ |
| 9 | 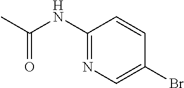 | 69%² 100%³ 18*80%⁴ |
| 10 | 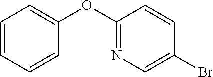 | 64%² 100%³ 52*100%⁴ |

Note:
¹method II-3
²yield of Suzuki Coupling
³yield of reduction
⁴yield of diazotization (and de-protection).

TABLE II-3

| Cpd | | | | MS | |
|---|---|---|---|---|---|
| No | Ex | Structures | MF/MW | (M⁺ + H) | IUPAC Name |
| 1244 | 1 | 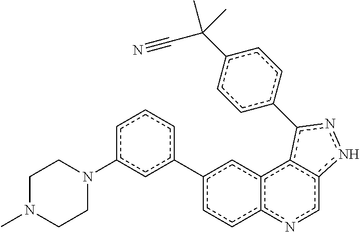 | C31H30N6/ 486.6 | 487 | 2-methyl-2-(4-(8-(3-(4- methylpiperazin-1- yl)phenyl)-3H- pyrazolo[3,4-c]quinolin-1- yl)phenyl)propanenitrile |
| 1245 | 2 | 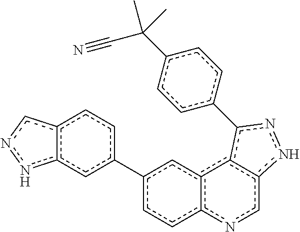 | C27H20N6/ 428.5 | 429.4 | 2-(4-(8-(1H-indazol-6-yl)- 3H-pyrazolo[3,4- c]quinolin-1-yl)phenyl)-2- methylpropanenitrile |
| 1246 | 3 | 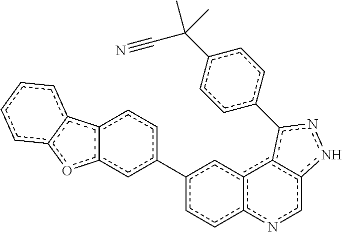 | C32H22N4O/ 478.5 | 479 | 2-(4-(8-(dibenzo[b,d]furan- 3-yl)-3H-pyrazolo[3,4- c]quinolin-1-yl)phenyl)-2- methylpropanenitrile |
| 1247 | 4 | 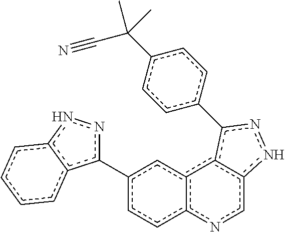 | C27H20N6/ 428.5 | 420 | 2-(4-(8-(1H-indazol-3-yl)- 3H-pyrazolo[3,4- c]quinolin-1-yl)phenyl)-2- methylpropanenitrile |
| 1248 | 5 | 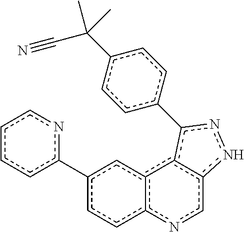 | C25H19N5/ 389.5 | 390 | 2-methyl-2-(4-(8-(pyridin- 2-yl)-3H-pyrazolo[3,4- c]quinolin-1- yl)phenyl)propanenitrile |
| 1249 | 6 | 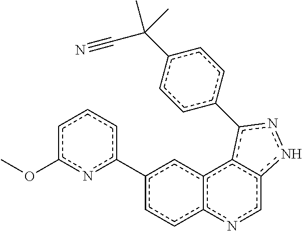 | C26H21N5O/ 419.5 | 479 | 2-(4-(8-(6- methoxypyridin-2-yl)- 3H-pyrazolo[3,4-c]quinolin- 1-yl)phenyl)-2- methylpropanenitrile |
| 1250 | 7 | 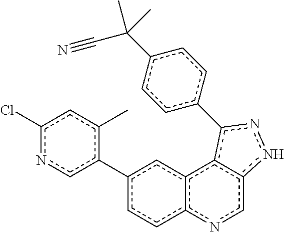 | C26H20ClN5/ 437.9 | 427 | 2-(4-(8-(6-chloro-4- methylpyridin-3-yl)-3H- pyrazolo[3,4-c]quinolin-1- yl)phenyl)-2- methylpropanenitrile |
| 1251 | 8 | 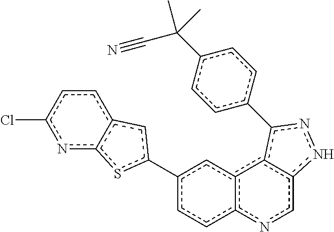 | C27H18ClN5S/ 480 | 480 | 2-(4-(8-(6- chlorothieno[2,3-b]pyridin- 2-yl)-3H-pyrazolo[3,4- c]quinolin-1-yl)phenyl)-2- methylpropanenitrile |
| 1252 | 9 | 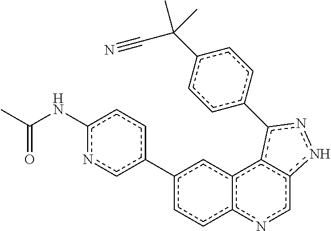 | C27H22N6O/ 446.5 | 447 | N-(5-(1-(4-(2- cyanopropan-2-yl)phenyl)- 3H-pyrazolo[3,4- c]quinolin-8-yl)pyridin-2- yl)acetamide |
| 1253 | 10 | 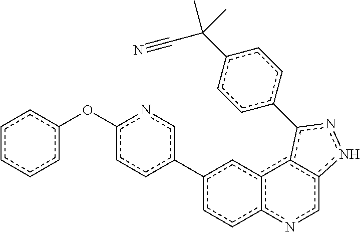 | C31H23N5O/ 481.5 | 482 | 2-methyl-2-(4-(8-(6- phenoxypyridin-3-yl)-3H- pyrazolo[3,4-c]quinolin-1- yl)phenyl)propanenitrile |

Synthetic Procedures for Preparing Compounds in Table II-3.

### 2-methyl-2-(4-((3-nitro-6-(4,4,5,5-tetramethyl-1,3,2-dioxaborolan-2-yl)quinolin-4-yl)methyl)phenyl)propanenitrile (34)

A mixture of 23, 2-(4-((6-bromo-3-nitroquinolin-4-yl)methyl)phenyl)-2-methyl propanenitrile (0.21 g, 0.51 mmol), bis(pinacolato)diboron (0.14 g, 0.55 mmol), PdCl₂(dppf) (0.02 g, 0.05 eq.), KOAc (0.15 g, 1.53 mmol) and toluene (15 ml) was degassed and charged with dry N₂, and heated to reflux for 8 h. After solvent was removed under reduced pressure, the residue was purified by column chromatography (silica gel, EA:PE 1:5) to give 0.18 g boronate ester 34 as white solid (yield, 78%). MS (m/z) (M⁺+H): 458, 376 (as acid form).

### Example 1

### 2-methyl-2-(4-(8-(3-(4-methylpiperazin-1-yl)phenyl)-3H-pyrazolo[3,4-c]quinolin-1-yl)phenyl)propanenitrile (1244)

A mixture of compound 34 (0.20 g, 0.44 mmol), 1-(3-bromophenyl)-4-methyl piperazine (0.17 g, 1.1 eq.), Pd(PPh₃)₄ (0.03 g, 0.05 eq.), Na₂CO₃ (0.14 g, 3 eq.), toluene (20 ml) and H₂O (2 ml) was degassed and protected with N₂, and heated to reflux for 12 h. The mixture was cooled, diluted with water (5 ml) and extracted with EA (10 ml). Organic layer was dried over Na₂SO₄, concentrated and purified by column chromatography (silica gel, EA:PE 1:5) to give 35a, 2-(4-((6-(9H-carbazol-2-yl)-3-nitroquinolin-4-yl)methyl)phenyl)-2-methylpropanenitrile (75 mg, 34%) as yellow solid MS (m/z) (M⁺+H): 506.

A mixture of 35a (75 mg, 0.15 mmol), Pd/C (15 mg) and MeOH (20 ml) was hydrogenated for 2 h. The mixture was filtrated and evaporated to dryness to give 36a, 2-(4-((3-amino-6-(9H-carbazol-2-yl)quinolin-4-yl)methyl)phenyl)-2-methylpropanenitrile (60 mg, 85%). MS (m/z) (M⁺+H): 476.

To a solution of 36a (60 mg, 0.03 mmol) in 10 ml of AcOH was added a solution of NaNO₂ (9 mg in 0.5 ml of H₂O) at room temperature. The mixture was stirred overnight. Solvent was removed under reduced pressure, and the residue was purified by column chromatography (silica gel, EA/PE 1:1) to give 37a, 2-methyl-2-(4-(8-(3-(4-methylpiperazin-1-yl)phenyl)-3H-pyrazolo[3,4-c]quinolin-1-yl)phenyl)propanenitrile (23 mg, 38%). MS (m/z) (M⁺+H): 487. ¹H-NMR (DMSO-d₆) δ 14.30 (s, 1H), 9.28 (s, 1H), 8.30 (s, 1H), 8.17 (d, 1H, J=8.29 Hz), 7.95˜7.91 (m, 3H), 7.76 (d, 2H, J=8.29 Hz), 7.26 (t, 1H, J=7.80 Hz), 7.14 (s, 1H), 6.97 (t, 2H, J=8.78 Hz), 3.19˜3.14 (m, 8H), 2.22 (s, 3H), 1.78 (s, 6H).

### Example 2

### 2-(4-(8-(1H-indazol-6-yl)-3H-pyrazolo[3,4-c]quinolin-1-yl)phenyl)-2-methylpropanenitrile (1245)

To a solution of compound 34 (160 mg, 0.35 mmol) in DMF (5 mL) was added 6-bromo-1H-indazole (103 mg, 0.53 mmol), 1M Na₂CO₃ (91 mg, 1.05 mmol, in 1.0 mL water) and Pd(PPh₃)₄ (39 mg, 0.035 mmol). The reaction mixture was protected with N₂, and stirred under microwave for 30 min at 100° C. The mixture was diluted with water (10 mL), extracted with DCM (3×20 mL), washed with brine, dried over Na₂SO₄, filtered, and concentrated. The resulting residue was purified by column chromatography (DCM:Methanol 80:1 to 60:1) to give 35b, 2-(4-((6-(1H-indazol-6-yl)-3-nitroquinolin-4-yl)methyl)phenyl)-2-methylpropanenitrile (70 mg, 45%) as a light yellow solid.

To a suspension of 35b (67 mg, 0.15 mmol) in acetic acid (5 mL) and H₂O (2 mL) was added rapidly a solution of TiCl₃ (1.5 mL, 13% in a 20% HCl solution). After stirring for 30 min at room temperature, a solution of 15% NaOH was added until pH 9. The reaction mixture was extracted with DCM, washed with brine, dried over MgSO₄ and concentrated to give 36b, 2-(4-((3-amino-6-(1H-indazol-6-yl)quinolin-4-yl)methyl)phenyl)-2-methylpropanenitrile (51 mg. 82%).

To a solution of 36b (42 mg, 0.1 mmol) in toluene (50 mL) was added KOAc (30 mg, 0.3 mmol) and acetic anhydride (21 mg, 0.2 mmol) under stirring. The mixture was monitored by HPLC for the consumption of starting material. To the mixture was added isoamylnitrite (12 mg, 0.12 mol). The resulting mixture was heated to 80° C. and stirred for 18 h. The solvent was removed under reduced pressure. The residue was purified by chromatography (silica gel, EA:PE1:5 to 1:1) to give acetylated product (26 mg, 50%) as a light yellow solid. To the solution of above acetylated product (26 mg, 0.05 mmol) in EtOH (10 mL) was added K₂CO₃ (14 mg, 0.1 mmol). The reaction mixture was refluxed overnight. The solution was concentrated under reduced pressure. The residue was purified by column chromatography (MeOH:DCM1:80 to 1:30) to give 37b (17 mg, 80%) as a light yellow solid. MS (m/z) (M⁺+H): 429. ¹H-NMR (δ, DMSO-d6, 400 MHz, ppm), 11.19 (s, 1H), 9.24 (s, 1H), 8.33 (d, 1H, J=1.95 Hz), 8.17 (d, 1H, J=8.77 Hz), 7.98-8.01 (m, 1H), 7.92 (d, 2H, J=8.29 Hz), 7.80-7.84 (m, 3H), 7.44 (d, 1H, J=8.78 Hz), 7.35-7.38 (m, 2H), 6.48 (s, 1H), 1.80 (s, 6H).

### Example 3

### 2-(4-(8-(dibenzo[b,d]furan-3-yl)-3H-pyrazolo[3,4-c]quinolin-1-yl)phenyl)-2-methylpropanenitrile (1246)

A mixture of compound 34 (200 mg, 0.44 mmol, 1 eq), 3-bromodibenzofuran (118 mg, 0.48 mmol, 1.1 eq), Pd(PPh₃)₄ (10 mg, 0.008 mmol, 0.02 eq), Na₂CO₃ (137 mg, 1.3 mmol, 3 eq), toluene (10 mL) and H₂O (2 mL) was degassed, protected with N₂, and refluxed for 12 hrs. After cooled to room temperature, the mixture was diluted with water, extracted with EA. The organic phase was dried over MgSO₄ and filtered. The filtrate was concentrated and purified by column chromatography (silica gel) to give 35c, 2-(4-((6-(dibenzo[b,d]furan-3-yl)-3-nitroquinolin-4-yl)methyl)phenyl)-2-methyl propanenitrile (156 mg, 50%).

A mixture of 35c (235 mg, 0.50 mmol, 1 eq) and Pd/C (35 mg, 15% eq) in THF (10 mL) was stirred under H₂ for 1 h. The mixture was filtered and the solvent was removed to give 36c, 2-(4-((6-(dibenzo[b,d]furan-3-yl)-3-aminoquinolin-4-yl)methyl)phenyl)-2-methyl propanenitrile (158 mg, 70%).

A mixture of 36c (100 mg, 0.23 mmol, 1 eq), AcOK (38 mg, 0.46 mmol, 2 eq), acetic anhydride (74 mg, 0.69 mmol, 3 eq) in toluene (10 mL) was stirred at RT for 2 h. tert-butylnitrite (29 mg, 1.28 mmol, 1.2 eq) was added and the mixture was heated at 60° C. for 12 h. The solvent was removed under vacuum. K₂CO₃ (66 mg, 0.46 mmol, 2 eq) and EtOH (10 mL) was added. The mixture was refluxed for 1 h. The solvent was removed in vacuum. The residue was dissolved in EA and washed with water. The organic phase was dried over MgSO₄ and evaporated. The mixture was purified by chromatography to give 37c, 2-(4-(8-(dibenzo[b,d]furan-3-yl)-3H-pyrazolo[3,4-c]quinolin-1-yl)phenyl)-2-methylpropanenitrile (22 mg, two step: 20%). MS (m/z) (M⁺+H): 479. ¹H-NMR (δ, DMSO-d6, 400 MHz, ppm): 14.23 (s, 1H), 9.30 (s, 1H), 8.39 (s, 1H), 7.81˜8.25 (m, 3H), 7.81˜7.94 (m, 5H), 7.39˜7.70 (m, 5H), 1.82 (s, 6H).

### Example 4

### 2-(4-(8-(1H-indazol-3-yl)-3H-pyrazolo[3,4-c]quinolin-1-yl)phenyl)-2-methylpropanenitrile (1247)

To a solution of compound 34 (160 mg, 0.35 mmol) in DMF (5 mL) was added 3-iodo-1H-indazole (129 mg, 0.53 mmol), 1M Na₂CO₃ (91 mg, 1.05 mmol, in 1.0 mL water) and Pd(PPh₃)₄ (39 mg, 0.035 mmol). The reaction mixture was stirred under microwave at 100° C. for 30 min, then diluted with water (10 mL), extracted with DCM (3×20 mL). Organic layer was washed with brine, dried over Na₂SO₄, filtered. The filtrate was concentrated. The residue was purified by column chromatography (DCM:Methanol 80:1 to 60:1) to give 35d, 2-(4-((3-nitro-6-(1H-indazol-3-yl)quinolin-4-yl)methyl)phenyl)-2-methylpropanenitrile (51 mg, 34%) as a light yellow solid.

To a suspension of 35d (50 mg, 0.11 mmol) in acetic acid (5 mL) and H₂O (2 mL) was added rapidly a solution of TiCl₃ (1.1 mL, 13% in a 20% HCl solution). After stirring for 30 min at room temperature, a solution of 15% NaOH was added until pH 9. The reaction mixture was extracted with DCM, dried over MgSO₄ and concentrated to give 36d, 2-(4-((3-amino-6-(1H-indazol-3-yl)quinolin-4-yl)methyl)phenyl)-2-methyl propane nitrile (40 mg, 86%).

To a solution of 36d (40 mg, 0.1 mmol) in toluene (50 mL) was added KOAc (30 mg, 0.3 mmol) and acetic anhydride (21 mg, 0.2 mmol) under stirring. The reaction was monitored by HPLC for the consumption of starting material. To the reaction mixture was charged isoamylnitrite (12 mg, 0.12 mol). The resulting mixture was heated to 80° C. and stirred for 18 h, at which time HPLC indicated the reaction was complete. The solvent was concentrated and the residue was purified by silica gel column (EA:PE1:5 to 1:1) to give acetylated product (24 mg, 50%) as a light yellow solid. To the solution of above product (24 mg, 0.05 mmol) in EtOH (10 mL) was added K₂CO₃ (14 mg, 0.1 mmol). The reaction mixture was refluxed overnight, concentrated and purified by column (MeOH:DCM1:80 to 1:30) to give 37d (16 mg, 75%) as a light yellow solid. MS (m/z) (M⁺+H): 420. ¹H-NMR (δ, MeOH-D4, 400 MHz, ppm), 9.67 (s, 1H), 8.26(s, 1H), 8.16 (d, 1H, J=8.00 Hz), 8.08 (d, 1H, J=8.80 Hz), 7.82 (s, 4H), 7.56 (s, 1H), 7.28-7.32 (m, 2H), 7.19 (d, 1H, J=8.80 Hz), 1.82 (s, 6H).

### Example 5

### 2-methyl-2-(4-(8-(pyridine-2-yl)-3H-pyrazolo[3,4-c]quinolin-1-yl)phenyl)propanenitrile (1248)

A mixture of compound 34 (0.18 g, 0.4 mmol), 2-bromopridine (100 mg, 1.1 eq.), Pd(PPh₃)₄ (20 mg, 0.05 eq.), Na₂CO₃ (126 mg, 1.18 mmol), toluene (20 ml) and H₂O (2 ml) was degassed and protected with N₂ and heated to reflux for 12 h. After cooled to room temperature, the mixture was washed diluted water (5 ml) and extracted with EA (10 ml). The organic phase was dried over Na₂SO₄, filtered. The filtrate was concentrated and purified by column chromatography (silica gel, EA:PE 1:5) to give 35e, 2-methyl-2-(4-((3-nitro-6-(pyridine-2-yl)quinolin-4-yl)methyl)phenyl)propanenitrile, as yellow solid (54 mg, 34%). MS (m/z) (M⁺+H): 409.

A mixture of 35e (54 mg, 0.13 mmol) and Pd/C (25 mg) in MeOH (20 ml) was hydrogenated for 2 h. The mixture was filtered, solvent was removed to give 36e, 2-methyl-2-(4-((3-amino-6-(pyridine-2-yl)quinolin-4-yl)methyl)phenyl)propanenitrile (33 mg, 66%). LC/MS (M/Z) M++H): m/z 379⁺.

To a solution of 36e (33 mg, 0.087 mmol) in 10 ml of AcOH was added a solution of NaNO₂ (6 mg in 0.5 ml of H₂O) at room temperature. The mixture was stirred overnight. Solvent was removed under reduced pressure. The residue was purified by column chromatography (silica gel, EA/PE 1:1) to give 37e, 2-methyl-2-(4-(8-(pyridine-2-yl)-3H-pyrazolo[3,4-c]quinolin-1-yl)phenyl)propanenitrile (18 mg, 53%). MS (m/z) (M⁺+H): 390. ¹H-NMR (δ, ppm, DMSO-d₆, 400 MHz) 14.24 (s, 1H), 9.31 (s, 1H), 8.86 (s, 1H), 8.61 (d, 1H, J=4.38 Hz), 8.32 (d, 1H, J=7.31 Hz), 8.22 (d, 1H, J=8.77 Hz), 7.91˜7.78 (m, 6H), 7.37˜7.34 (m, 1H), 1.81 (s, 6H).

### Example 6

### 2-(4-(8-(6-methoxypyridin-2-yl)-3H-pyrazolo[3,4-c]quinolin-1-yl)phenyl)-2-methylpropanenitrile (1249)

A mixture of compound 34 (200 mg, 0.44 mmol, 1 eq), 2-bromo-6-methoxypyridine (90 mg, 0.48 mmol, 1.1 eq), Pd(PPh₃)₄ (10 mg, 0.008 mmol, 0.02 eq), Na₂CO₃ (137 mg, 1.3 mmol, 3 eq), toluene (10 mL) and H₂O (2 mL) was degassed and protected with N₂. The mixture was refluxed for 12 h. After cooling to room temperature, the mixture was diluted with water and extracted with EA. The organic phase was dried over MgSO₄, filtered. The filtrate was concentrated and purified by column chromatography (silica gel) to give 35f, 2-(4-((6-(6-methoxypyridin-2-yl)-3-nitroquinolin-4-yl)methyl)phenyl)-2-methyl propanenitrile (120 mg, 63%).

A mixture of 35f (120 mg, 0.26 mmol, 1 eq) and Pd/C (20 mg, 15% eq) in THF (10 mL) was stirred under H₂ for 1 h. The mixture was filtered and the solvent was removed to give 36f, 2-(4-((6-(6-methoxypyridin-2-yl)-3-aminoquinolin-4-yl)methyl)phenyl)-2-methyl propanenitrile (90 mg, 81%).

A mixture of 36f (90 mg, 0.22 mmol, 1 eq), AcOK (30 mg, 0.44 mmol, 2 eq), acetic anhydride (70 mg, 0.66 mmol, 3 eq) in toluene (10 mL) was stirred at RT for 2 h. tert-butylnitrite (29 mg, 1.28 mmol, 1.2 eq) was added and the mixture was heated at 60° C. for 12 h. The solvent was removed under vacuum. K₂CO₃ (66 mg, 0.44 mmol, 2 eq) and EtOH (10 mL) was added. The mixture was refluxed for 1 h. The solvent was removed in vacuo. The residue was dissolved in EA and washed with brine, dried over MgSO₄, filtered and evaporated. The residue was purified by chromatography to give 37f, 2-(4-(8-(6-methoxypyridin-2-yl)-3H-pyrazolo[3,4-c]quinolin-1-yl)phenyl)-2-methyl propanenitrile (20 mg, of two step: 21%). MS (m/z) (M⁺+H): 479. ¹H-NMR (δ, ppm, DMSO-d6, 400 MHz): 9.30 (s, 1H), 8.96 (s, 1H), 8.28 (s, 1H), 8.23 (s, 1H), 7.90 (d, 1H, J=7.32 Hz), 7.76(s, 1H), 7.53 (d, 1H, J=7.33 Hz), 6.79 (1H, J=7.81 Hz), 3.77 (s, 1H), 1.78 (s, 6H).

### Example 7

### 2-(4-(8-(6-chloro-4-methylpyridin-3-yl)-3H-pyrazolo[3,4-c]quinolin-1-yl)phenyl)-2-methylpropanenitrile (1250)

A mixture of compound 34 (300 mg, 0.66 mmol, 1 eq), 5-bromo-2-chloro-4-methylpyridine (165 mg, 0.8 mmol), Pd(PPh₃)₄ (81 mg, 0.035 mmol), Na₂CO₃ (210 mg, 1.98 mmol), toluene (10 mL) and H₂O (2 mL) was degassed and protected with N₂. The mixture was refluxed for 12 h. After cooling to room temperature, the mixture was diluted with water and extracted with EA. The organic phase was dried over MgSO₄ and filtered. The filtrate was concentrated and purified by column chromatography (silica gel) to give 35g, 2-(4-((6-(6-chloro-4-methylpyridin-3-yl)-3-nitroquinolin-4-yl)methyl)phenyl)-2-methylpropanenitrile. Yield: 59 mg, 20%.

A mixture of 35g (59 mg) and Pd/C (9 mg, 15% eq) in THF (10 mL) was stirred under H₂ for 1 h. The mixture was filtered and the solvent was removed to give 36g, 2-(4-((6-(6-chloro-4-methylpyridin-3-yl)-3-aminoquinolin-4-yl)methyl)phenyl)-2-methyl propanenitrile (44 mg, 80%).

To a solution of 36g (44 mg, 0.1 mmol, 1 eq.) in toluene (10 ml) was added KOAc (20 mg, 2 eq.) and Ac₂O (30 mg, 3 eq). The mixture was stirred at room temperature for 2 h. To the mixture was added tert-Butylnitrite (12 mg, 1.2 eq.) and the reaction mixture was heated to 60° C. overnight. The volatile materials were removed and the residue was purified by column chromatography (silica gel, EA:PE 1:5) to give 37g, 2-(4-(8-(6-chloro-4-methylpyridin-3-yl)-3H-pyrazolo[3,4-c]quinolin-1-yl)phenyl)-2-methylpropanenitrile (20 mg, 44%). MS (m/z) (M⁺+H): 427. ¹H-NMR (δ, CDCl₃, 400 MHz, ppm): 9.27 (s, 1H), 8.32 (d, 1H, J=8.43 Hz), 8.24 (s, 1H), 8.10 (d, 1H, J=1.83 Hz), 7.81(d, 1H, J=8.43 Hz), 7.65(d, 1H, J=8.43 Hz), 7.60 (m, 1H), 7.27 (s, 1H), 2.28 (s, 1H), 1.72 (s, 6H).

### Example 8

### 2-(4-(8-(6-chlorothieno[2,3-b]pyridine-2-yl)-3H-pyrazolo[3,4-c]quinolin-1-yl)phenyl)-2-methylpropanenitrile (1251)

A mixture of compound 34 (20 mg, 0.0437 mmol), 1-(3-bromophenyl)-4-(methylsulfonyl)piperazine (12 mg, 0.048 mmol), Pd(PPh₃)₄ (2.5 mg, 0.05 eq.), Na₂CO₃ (14 mg, 0.131 mmol), toluene (5 ml) and H₂O (0.5 ml) was degassed and protected with N₂ and heated to reflux for 12 h. After cooled to room temperature, the mixture was diluted with water (2 ml) and extracted with EA (5 ml). The organic phase was dried over Na₂SO₄, and filtered. The filtrate was concentrated and purified by column chromatography (silica gel, EA:PE 1:5) to give 35h, 2-methyl-2-(4-((6-(6-methylthieno[2,3-b]pyridine-2-yl)-3-nitroquinolin-4-yl)methyl)phenyl)propanenitrile as yellow powder (18 mg, 82.5%). LC/MS (M/Z) M++H): 499.

A mixture of 35h (180 mg, 0.36 mmol) and Pd/C (25 mg) in THF (20 ml) was hydrogenated for 3 h. The mixture was filtered. The solvent was removed to give 36h, 2-methyl-2-(4-((6-(6-methylthieno[2,3-b]pyridine-2-yl)-3-aminoquinolin4-yl)methyl)phenyl)propanenitrile as off-white powder. Yield: 150 mg, 88%. MS (m/z) (M⁺+H): 469.

To a solution of 36h (70 mg, 0.15 mmol) in toluene (10 ml) was added KOAc (29.3 mg, 0.3 mmol) and acetic anhydride (45.7 mg, 0.45 mmol). The mixture was stirred at rt for 2 h. To the reaction mixture was added tert-Butyl nitrite (20 mg, 0.19 mmol). The resulting mixture was heated to 80° C. and stirred for 18 h. The solvent was removed. The residue was purified by flash chromatography (silica gel, EA/PE 1:1) to give 2-(4-(3-acetyl-8-(6-chlorothieno[2,3-b]pyridine-2-yl)-3H-pyrazolo[3,4-c]quinolin-1-yl)phenyl)-2-methyl propanenitrile. Yield: 23 mg, 29%. LC/MS (M/Z) M++H): 521. To a solution of above compound (23 mg, 0.044 mmol) in EtOH (5 mL) was added K₂CO₃ (12 mg, 0.088 mmol). The reaction mixture was stirred for 3 h at rt. The solution was filtered, concentrated and purified by flash chromatograph (silica gel, EA/Hex1:2) and re-crystallized to give 37h, 2-(4-(8-(6-chlorothieno[2,3-b]pyridine-2-yl)-3H-pyrazolo[3,4-c]quinolin-1-yl)phenyl)-2-methylpropanenitrile as yellow powder (4 mg, 19%). MS (m/z) (M⁺+H): 480. ¹H-NMR (δ, DMSO-d6, 400 MHz, ppm): 8.52 (s, 1H), 8.30 (m, 2H), 7.98 (m, 2H), 7.92 (d, 2H), 7.80 (d, 2H), 7.41(s, 1H), 7.32 (d, 2H), 1.91 (s, 6H).

### Example 9

### N-(5-(1-(4-(2-cyanopropan-2-yl)phenyl)-3H-pyrazolo[3,4-c]quinolin-8-yl)pyridine-2-yl)acetamide (1252)

A mixture of 34 (0.20 g, 0.44 mmol), N-(5-bromopyridin-2-yl)acetamide (110 mg, 1.2 eq.), PdCl₂(dppf) (20 mg, 0.05 eq.), K₂CO₃ (180 mg, 3 eq.), toluene (20 ml) and H₂O (10 ml) was degassed and protected with N₂ and heated to reflux for 12 h. The mixture was cooled to room temperature, diluted with water (5 ml) and extracted with EA (10 ml). The organic phase was dried over Na₂SO₄, and filtered. The filtrate was concentrated and purified by column chromatography (silica gel, EA:PE 1:5) to give 35i, N-(5-(4-(4-(2-cyanopropan-2-yl)benzyl)-3-nitroquinolin-6-yl)pyridine-2-yl)acetamide as a yellow solid. Yield: 140 mg, 69%. MS (m/z) (M⁺+H): 466.

A mixture of 35i (140 mg, 0.22 mmol) and Pd/C (22 mg) and THF (30 ml) was hydrogenated for 2 h. the mixture was filtered and concentrated to give 36i, N-(5-(4-(4-(2-cyanopropan-2-yl)benzyl)-3-aminoquinolin-6-yl)pyridine-2-yl)acetamide (96 mg, 100%). MS (m/z) (M⁺+H): 436.

To a solution of 36i (96 mg, 0.22 mmol) in toluene (30 ml) was added KOAc (33 mg, 1.5 eq.) and Ac₂O (34 mg, 1.5 eq.). The mixture was stirred at room temperature for 2 h, then was added t-BuONO (25 mg, 1.1 eq.). The mixture was heated to 80° C. overnight. After cooled to RT, solvent was removed and residue was purified by column chromatography (silica gel, EA:PE 1:5) to give N-(5-(3-acetyl-1-(4-(2-cyanopropan-2-yl)phenyl)-3H-pyrazolo[3,4-c]quinolin-8-yl)pyridine-2-yl)acetamide (19 mg, 18%), MS (m/z) (M⁺+H): 489. The mixture of above product (19 mg), K₂CO₃ (91 mg) and THF (20 ml) and H₂O (10 ml) was heated to refluxed for 2 h and cooled. The mixture was extracted with EA (2×20 ml). The organic phase was dried over MgSO₄, and filtered. The filtrate was purified by column chromatography (silica gel, 5% MeOH in CH₂Cl₂) to give 37i, N-(5-(1-(4-(2-cyano propan-2-yl)phenyl)-3H-pyrazolo[3,4-c]quinolin-8-yl)pyridine-2-yl)acetamide (7.5 mg, 44%). MS (m/z) (M⁺+H): 447. ¹H-NMR (δ, DMSO-d₆ ppm, 400 MHz) 10.63 (s, 1H), 9.29 (s, 1H), 8.57 (s, 1H), 8.29 (s, 1H), 8.21 (d, 1H, J=7.80 Hz), 8.15 (d, 1H, J=8.78 Hz), 7.98 (d, 2H, J=8.77 Hz), 7.93 (d, 2H, J=8.29 Hz), 7.80 (d, 2H, J=8.29 Hz), 2.10 (s, 3H), 1.78 (s, 6H).

### Example 10

### 2-methyl-2-(4-(8-(6-phenoxypyridin-3-yl)-3H-pyrazolo[3,4-c]quinolin-1-yl)phenyl)propanenitrile (1253)

A mixture of 34 (0.20 g, 0.44 mmol), 2-phenoxy-5-bromopyridine (120 mg, 1.1 eq.), PdCl₂(dppf) (20 mg, 0.05 eq.), K₂CO₃ (180 mg, 3 eq.), toluene (15 ml) and H₂O (15 ml) was degassed and protected by N₂ and heated to reflux for 12 h. The mixture was cooled to room temperature, diluted with water (5 ml) and extracted with EA (10 ml). The organic phase was dried over Na₂SO₄, and filtered. The filtrate was concentrated and purified by column chromatography (silica gel, EA:PE 1:5) to give 35J, 2-(4-((3-nitro-6-(6-phenoxypyridin-3-yl)quinolin-4-yl)methyl)phenyl)-2-methylpropanenitrile as a yellow solid. Yield: 140 mg, 64%. LC/MS (M/Z) M⁺+H): 501.

A mixture of 35J (140 mg, 0.28 mmol) and Pd/C (30 mg) in THF (40 ml) was hydrogenated for 2 h. The mixture was filtered. Solvent was to give 36J, 2-(4-((3-amino-6-(6-phenoxypyridin-3-yl)quinolin-4-yl)methyl)phenyl)-2-methyl propanenitrile (132 mg, 100%). MS (m/z) (M⁺+H): 471.

To a solution of 36J (132 mg, 0.28 mmol) in toluene (30 ml) was added KOAc (41 mg, 1.5 eq.) and Ac₂O (43 mg, 1.5 eq.). The mixture was stirred at room temperature for 2 h, and then t-BuONO (32 mg, 1.1 eq.) was added. The reaction mixture was heated to 80° C. overnight. After cooled to RT, solvent was removed, the residue was purified by column chromatography (silica gel, EA:PE 1:5) to give 2-(4-(3-acetyl-8-(6-phenoxypyridin-3-yl)-3H-pyrazolo[3,4-c]quinolin-1-yl)phenyl)-2-methylpropanenitrile (70 mg, 52%). MS (m/z) (M⁺+H): 524. The mixture of above product (70 mg, 0.13 mmol) and K₂CO₃ (116 mg), THF (20 ml) and H₂O (10 ml) was heated to refluxed for 2 h, extracted with EA (2×20 ml), dried over MgSO₄ and filtered. The filtrate was purified by column chromatography (silica gel, 5% MeOH in CH₂Cl₂) gave 37J, 2-methyl-2-(4-(8-(6-phenoxypyridin-3-yl)-3H-pyrazolo[3,4-c]quinolin-1-yl)phenyl)propanenitrile (64 mg, 100%). MS (m/z) (M⁺+H): 482. ¹H-NMR (δ, ppm, DMSO-d₆, 400 MHz) 14.23 (s, 1H), 9.28 (s, 1H), 8.37 (s, 1H), 8.17 (s, 1H), 8.02 (d, 2H, J=2.69 Hz), 7.92 (d, 1H, J=9.28 Hz), 7.88 (d, 2H, J=8.06 Hz), 7.77 (d, 2H, J=8.05 Hz), 7.45˜7.39 (m, 2H), 7.25˜7.21 (m, 2H), 7.14 (d, 2H, J=9.03 Hz), 7.07 (d, 2H, J=9.28 Hz), 1.75 (s, 6H).

II-4. Method 4 Modification of Pyrazolo[3,4-c]quinoline Derivatives

Suzuki coupling reaction of aryl halide 11 with 3-nitrophenyl boronic acid provided coupling compound (38). Reduction of nitro (38) to aniline (39), followed by alkylation, acylation, sulfonation, reductive amination, provided final compounds (40, A=CH). Final compounds (40, A=N) can be prepared from the similar approach by Suzuki coupling reaction of aryl halide 11 with 3-(boc)amino-pyridyl boronic and de-Boc protection, followed by alkylation, acylation, sulfonation, reductive amination. The reaction conditions and yields were summarized in the following Table, and the final compounds were summarized in Table II-4.

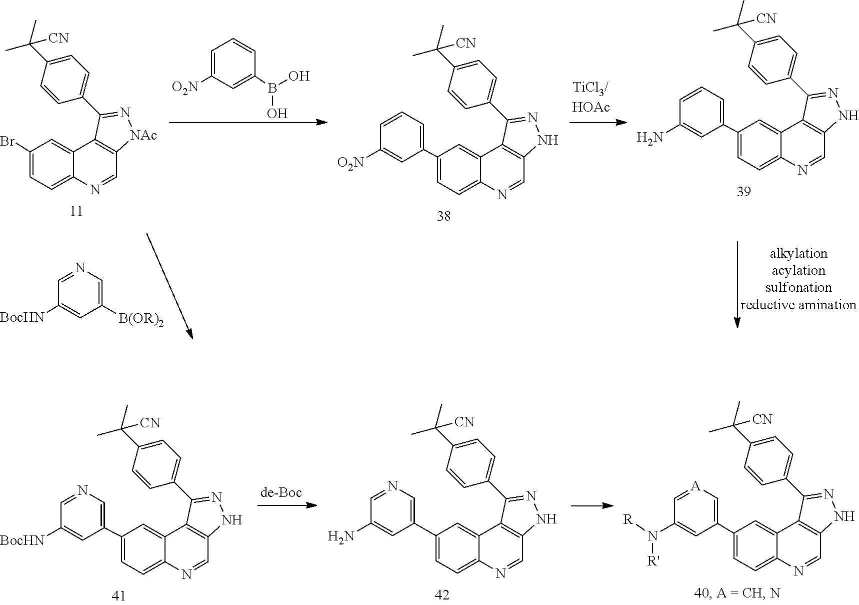

| Entry | 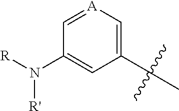 | Method | Yield (%) |
|---|---|---|---|
| 1 | 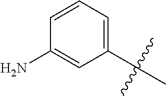 | Pd/C/H₂ | 82 |
| 2 | 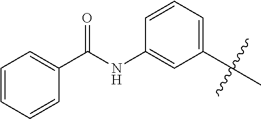 | BzCl/Net₃ | 11 |
| 3 | 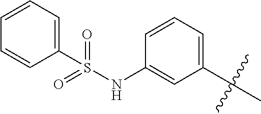 | PhSO₂Cl/Net₃ | 44 |
| 4 | 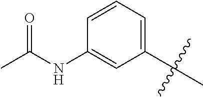 | AcCl/Net₃ | 27 |
| 5 | 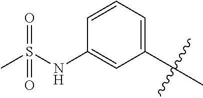 | MsCl/Net₃ | 30 |
| 6 | 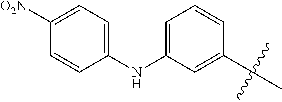 | 4-nitrofluoro- benzene DIEPA Microwave 190° C., 2hrs | 40 |
| 7 | 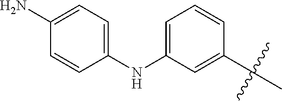 | Pd/C/H₂ | 70 |
| 8 | 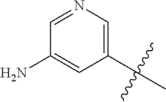 | HCl | 50 |
| 9 | 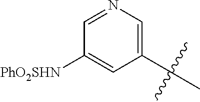 | PhSO₂Cl/NEt₃ | 60 |
| 10 | 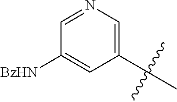 | BzCl/NEt₃ | 40 |
| 11 | 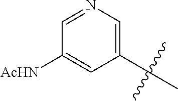 | AcCl/NEt₃ | 50 |
| 12 | 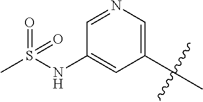 | MsCl/NEt₃ | 46 |
| 13 | 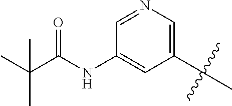 | PivCl/NEt₃ | 57 |
| 14 | 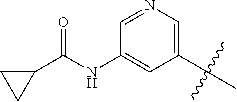 | Cyclopropylcar bonyl chloride/NEt₃ | 22 |

TABLE II-4

| Cpd | | | | MS | |
|---|---|---|---|---|---|
| No | Ex | Structure | MF/MW | (M⁺ + H) | IUPAC |
| 1254 | 1 | 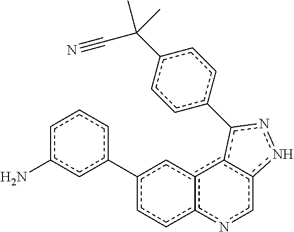 | C26H21N5/403.5 | 404 | 2-(4-(8-(3-aminophenyl)- 3H-pyrazolo[3,4- c]quinolin-1-yl)phenyl)-2- methylpropanenitrile |
| 1255 | 2 | 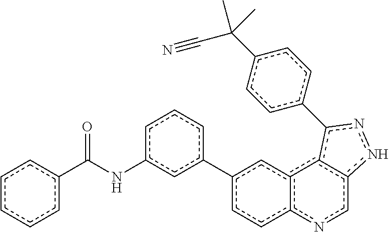 | C33H25N5O/507.6 | 508 | N-(3-(1-(4-(2- cyanopropan-2-yl)phenyl)- 3H-pyrazolo[3,4- c]quinolin-8- yl)phenyl)benzamide |
| 1256 | 3 | 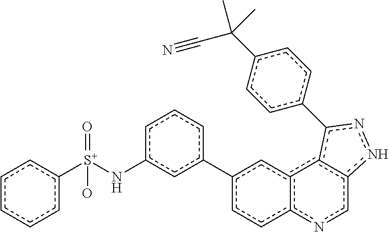 | C32H25N5O2S/543.6 | 544 | N-(3-(1-(4-(2- cyanopropan-2-yl)phenyl)- 3H-pyrazolo[3,4- c]quinolin-8- yl)phenyl)benzene- sulfonamide |
| 1257 | 4 | 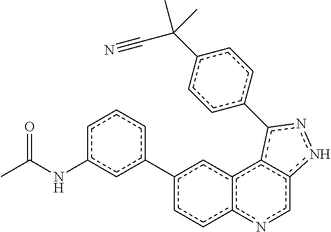 | C28H23N5O/445.5 | 446 | N-(3-(1-(4-(2- cyanopropan-2-yl)phenyl)- 3H-pyrazolo[3,4- c]quinolin-8- yl)phenyl)acetamide |
| 1258 | 5 | 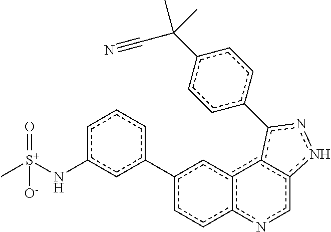 | C27H23N5O2S/481.6 | 482 | N-(3-(1-(4-(2- cyanopropan-2-yl)phenyl)- 3H-pyrazolo[3,4- c]quinolin-8- yl)phenyl)methane- sulfonamide |
| 1259 | 6 | 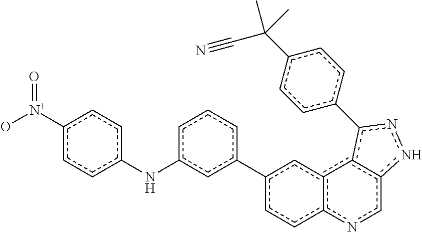 | C32H24N6O2/524.6 | 525 | 2-methyl-2-(4-(8-(3-(4- nitrophenylamino)phenyl)- 3H-pyrazolo[3,4- c]quinolin-1- yl)phenyl)propanenitrile |
| 1260 | 7 | 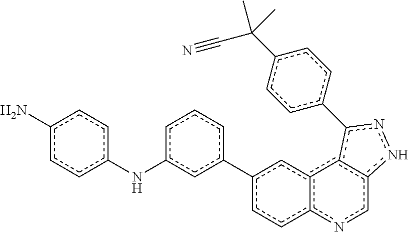 | C32H26N6/494.6 | 495 | 2-(4-(8-(3-(4- aminophenylamino) phenyl)-3H-pyrazolo[3,4- c]quinolin-1-yl)phenyl)-2- methylpropanenitrile |
| 1261 | 8 | 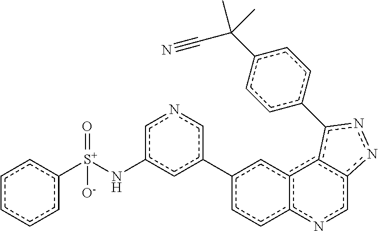 | C31H24N6O2S/544.6 | 545 | N-(5-(1-(4-(2- cyanopropan-2-yl)phenyl)- 3H-pyrazolo[3,4- c]quinolin-8-yl)pyridin-3- yl)benzenesulfonamide |
| 1262 | 9 | 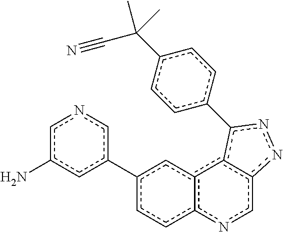 | C25H20N6/404.5 | 405 | 2-(4-(8-(5-aminopyridin-3- yl)-3H-pyrazolo[3,4- c]quinolin-1-yl)phenyl)-2- methylpropanenitrile |
| 1263 | 10 | 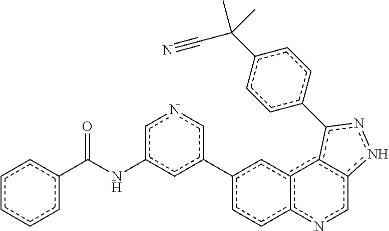 | C32H24N6O/508.6 | 510 | N-(5-(1-(4-(2- cyanopropan-2-yl)phenyl)- 3H-pyrazolo[3,4- c]quinolin-8-yl)pyridin-3- yl)benzamide |
| 1264 | 11 | 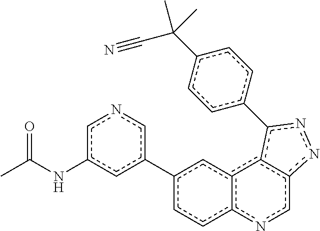 | C27H22N6O/446.5 | 447 | N-(5-(1-(4-(2- cyanopropan-2-yl)phenyl)- 3H-pyrazolo[3,4- c]quinolin-8-yl)pyridin-3- yl)acetamide |
| 1265 | 12 | 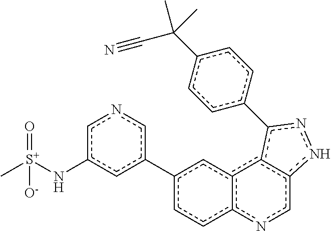 | C26H22N6O2S/482.6 | 483 | N-(5-(1-(4-(2- cyanopropan-2-yl)phenyl)- 3H-pyrazolo[3,4- c]quinolin-8-yl)pyridin-3- yl)methanesulfonamide |
| 1266 | 13 | 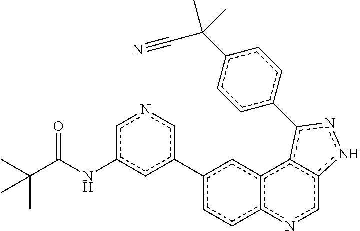 | C30H28N6O/488.6 | 489 | N-(5-(1-(4-(2- cyanopropan-2-yl)phenyl)- 3H-pyrazolo[3,4- c]quinolin-8-yl)pyridin-3- yl)pivalamide |
| 1267 | 14 | 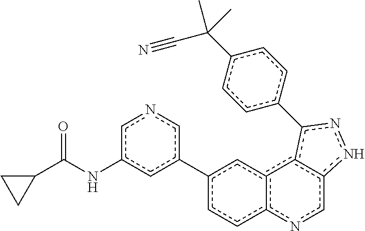 | C33H30N6O3 (with salt)/472.5 | 472 as base | N-(5-(1-(4-(2- cyanopropan-2-yl)phenyl)- 3H-pyrazolo[3,4- c]quinolin-8-yl)pyridin-3- yl)cyclopropane- carboxamide cyclopropanecarboxylate salt |

Synthetic Procedures for Preparing Compounds in Table II-4.

The procedures to make 38 and Examples 12 and 14 are the same as method II-1a and II-1b.

### 2-(4-(8-(3-nitrophenyl)-3H-pyrazolo[3,4-c]quinolin-1-yl)phenyl)-2-methyl propanenitrile (38)

To a solution of 11, 2-(4-(3-acetyl-8-bromo-3H-pyrazolo[3,4-c]quinolin-1-yl)phenyl)-2-methylpropanenitrile (200 mg, 0.46 mmol) in DMF (8 mL) was added 3-nitrophenylboronic acid (1 mmol), 1M Na₂CO₃ (150 mg, in 2 mL water) and Pd(PPh₃)₄ (50 mg, 0.046 mmol). The reaction mixture was purged with nitrogen and stirred under microwave for 30 min at 105-120° C. The reaction mixture was diluted with water (10 mL) and extracted with DCM (3×20 mL). The organic layer was washed with brine, dried over Na₂SO₄, filtered. The filtrate was concentrated. The resulting residue was purified by column chromatography (DCM:Methanol 80:1 to 60:1) to give 38 (140 mg, 70%) as a light yellow solid. MS (m/z) (M⁺+H): 434. ¹H-NMR: (δ, ppm, DMSO-d6, 400 Hz) 14.30(s, 1H), 9.33(s, 1H), 8.47(s, 1H), 8.40(s, 1H), 8.08-8.27(m, 4H), 7.97(d, 2H, J=8.04 Hz), 7.73-7.83(m, 3H), 1.78(s, 6H).

### Example 1

### 2-(4-(8-(3-aminophenyl)-3H-pyrazolo[3,4-c]quinolin-1-yl)phenyl)-2-methylpropanenitrile (39) (1254)

To a suspension of the 38 (90 mg, 0.2 mmol) in acetic acid (5 mL) and H₂O (2 mL) was added rapidly a solution of TiCl₃ (2 mL, 13% in a 20% HCl solution). After stirring for 15 min at room temperature, a solution of 15% NaOH was added until pH 9. The reaction mixture was extracted with AcOEt, dried over MgSO₄ and concentrated to give the 39 (69 mg, 82%). MS (m/z) (M⁺+H): 404. ¹H-NMR: (δ, ppm, MeOH-D4, 400 Hz) 9.14(s, 1H), 8.25(d, 1H, J=1.47 Hz), 8.10(d, 1H, J=8.43 Hz), 7.77-7.85 (m, 5H), 7.12(t, 1H, J=7.70 Hz), 6.91(s, 1H), 6.81(dd, 1H, J=10.73 Hz, J=26.64 Hz), 6.70(dd, 1H, J=12.57 Hz, J=28.47 Hz), 1.83(s, 6H).

General Procedure for Carbonylation or Sulfonylation (40)

To the solution of 39 (14 mg, 0.0347 mmol) in DCM (10 mL) and TEA (11 mg, 3 eq.) at room temperature, was added benzylchloride (15 mg, 3 eq.). The solution was stirred at room temperature for 1 h, and then was quenched by addition of H₂O (5 ml). The organic layer was collected and dried over MgSO₄, and filtered. The filtrate was concentrated and purified by preparative TLC (EA:PE 1:1) to give 2 mg of 40.

### Example 2

### N-(3-(1-(4-(2-cyanopropan-2-yl)phenyl)-3H-pyrazolo[3,4-c]quinolin-8-yl)phenyl)benzamide

11% yield MS (m/z) (M⁺+H): 508. ¹H-NMR: (δ, ppm, DMSO-d6, 400 Hz) 10.38 (s, 1H), 9.30 (s, 1H), 8.39 (s, 1H), 8.22 (d, 2H), 8.00 (d, 2H, J=8.00 Hz), 7.92(d, 3H, J=8.80 Hz), 7.80 (d, 2H), 7.61 (d, 1H), 7.55 (t, 2H), 7.43 (t, 1H), 7.34 (d, 1H), 1.64 (s, 6H).

### Example 3

### N-(3-(1-(4-(2-cyanopropan-2-yl)phenyl)-3H-pyrazolo[3,4-c]quinolin-8-yl)phenyl)benzenesulfonamide

44% yield. MS (m/z) (M⁺+H): 544. ¹H-NMR: (δ, ppm, DMSO-d6, 300 Hz) 14.24 (s, 1H), 10.43 (s, 1H), 9.29 (s, 1H), 8.30 (s, 1H), 8.20 (d, 1H, J=8.80 Hz), 7.91 (d, 2H, J=8.07 Hz), 7.73-7.82 (m, 5H), 7.44-7.57 (m, 4H), 7.22-7.30 (m, 2H), 7.01 (d, 1H, J=7.33 Hz), 1.78 (s, 6H).

### Example 4

### N-(3-(1-(4-(2-cyanopropan-2-yl)phenyl)-3H-pyrazolo[3,4-c]quinolin-8-yl)phenyl)acetamide

27% yield. MS (m/z) (M⁺+H): 446. ¹H-NMR: (δ, ppm, DMSO-d6, 400 Hz) 10.06 (s, 1H), 9.29 (s, 1H), 8.35 (s, 1H), 8.20 (d, 1H, J=8.79 Hz), 8.04 (s, 1H), 7.90 (t, 2H, J=8.43 Hz), 7.85 (t, 2H, J=8.43 Hz), 7.45 (d, 1H), 7.36 (t, 1H, J=8.07 Hz), 7.28(d, 1H), 2.08 (s, 3H), 1.77 (s, 6H).

### Example 5

### N-(3-(1-(4-(2-cyanopropan-2-yl)phenyl)-3H-pyrazolo[3,4-c]quinolin-8-yl)phenyl)methanesulfonamide (1258)

30% yield. MS (m/z) (M⁺+H): 482. ¹H-NMR: (δ, ppm, DMSO-d6, 400 Hz) 9.28 (s, 1H), 8.31 (d, 1H), 8.20 (s, 11-1), 8.18 (s, 1H), 7.90-7.76 (m, 5H), 7.42 (s, 1H), 7.35-7.31 (m, 1H), 7.21-7.19 (d, 1H, J=8 Hz), 7.15-7.13 (d, 1H, J=8 Hz), 2.92 (s, 3H), 1.78 (s, 6H).

### Example 6

### 2-methyl-2-(4-(8-(3-(4-nitrophenylamino)phenyl)-3H-pyrazolo[3,4-c]quinolin-1-yl)phenyl)propanenitrile (1259)

40% yield. MS (m/z) (M⁺+H): 525. ¹H-NMR: (δ, ppm, DMSO-d6, 400 Hz) 9.63 (s, 1H), 8.50 (d, 2H, J=9.17 Hz), 8.32 (d, 2H, J=8.80 Hz), 8.25(d, 2H, J=9.89 Hz), 7.89-8.00 (m, 5H), 7.07 (t, 1H, J=8.06 Hz), 6.83 (s, 1H), 6.69 (d, 1H, J=7.33 Hz), 6.53 (d, 1H, J=8.06 Hz), 5.21 (s, 1H), 1.81 (s, 6H).

### Example 7

### 2-(4-(8-(3-(4-aminophenylamino)phenyl)-3H-pyrazolo[3,4-c]quinolin-1-yl)phenyl)-2-methylpropanenitrile (1260)

70% yield. MS (m/z) (M⁺+H): 495. ¹H-NMR: (δ, ppm, DMSO-d6, 400 Hz) 9.25 (s, 1H), 8.28 (s, 1H), 8.19 (d, 1H, J=8.30 Hz), 7.95-7.85 (m, 5H), 7.53 (d, 2H), 7.07 (t, 1H, J=7.8 Hz), 6.84 (s, 1H), 6.79 (d, 2H), 6.72 (d, 1H, J=7.3 Hz), 6.55 (s, 1H), 5.54 (s, 1H), 5.20 (s, 1H), 1.81 (s, 6H).

### Example 9

### 2-(4-(8-(5-aminopyridin-3-yl)-3H-pyrazolo[3,4-c]quinolin-1-yl)phenyl)-2-methylpropanenitrile (42) (1262)

To a solution of 41 (152 mg, 0.3 mmol) in MeOH (25 mL) was added con. HCl (25uL, 3.0 mmol). The reaction mixture was stirred for 5 h at 20° C. To the above reaction mixture was added TEA until PH 8 and concentrated. The resulting residue was purified by column chromatography (DCM:Methanol 30:1 to 10:1) to give 42 (Example 8) (97 g, 80%) as a light yellow solid. MS (m/z) (M⁺+H): 405. ¹H-NMR: (δ, ppm, MeOH-D4, 400 Hz) 9.23 (s, 1H), 8.17-8.33 (m, 2H), 7.80-7.94 (m, 7H), 7.22-7.26 (m, 1H), 1.83 (s, 6H).

General Procedure for Carbonylation or Sulfonylation of 42 (43)

To a solution of 42 (20 mg, 0.05 mmol) in DCM (5 mL) was added Py (160 mg, 2.0 mmol) and PhSO₂Cl (8.8 mg, 0.05 mmol) at rt. The reaction mixture was stirred for 3 h at rt. The mixture was concentrated and purification by chromatography (DCM:Methanol 50:1 to 30:1) to give 43 (12 mg, 80%) as a light yellow solid.

### Example 8

### N-(5-(1-(4-(2-cyanopropan-2-yl)phenyl)-3H-pyrazolo[3,4-c]quinolin-8-yl)pyridine-3-yl)benzenesulfonamide (1261)

60% yield. MS (m/z) (M⁺+H): 545. ¹H-NMR: (δ, ppm, MeOH-D4, 400 Hz) 9.82 (s, 1H), 8.52 (d, 1H, J=2.19 Hz), 8.41 (d, 1H, J=8.80 Hz), 8.20 (s, 1H), 8.13 (d, 1H, J=8.00 Hz), 7.82-8.05 (m, 9H), 7.55-7.57 (m, 1H), 7.42-7.46 (m, 1 Hz), 1.84 (s, 6H).

### Example 10

### N-(5-(1-(4-(2-cyanopropan-2-yl)phenyl)-3H-pyrazolo[3,4-c]quinolin-8-yl)pyridine-3-yl)benzamide (1263)

40% yield. MS (m/z) (M⁺+H): 510. ¹H-NMR: (δ, ppm, MeOH-D4, 400 Hz) 9.19 (s, 1H), 8.15-8.27 (m, 2H), 8.00 (d, 2H, J=8.43 Hz), 7.93 (d, 2H, J=5.68 Hz), 7.77-7.85 (m, 6H), 7.54-7.56 (m, 1H), 7.44 (t, 1H, J=7.70 Hz), 7.22 (d, 1H, J=4.03 Hz), 1.81 (s, 6H).

### Example 11

### N-(5-(1-(4-(2-cyanopropan-2-yl)phenyl)-3H-pyrazolo[3,4-c]quinolin-8-yl)pyridine-3-yl)acetamide (1264)

50% yield. MS (m/z) (M⁺+H): 447. ¹H-NMR: (δ, ppm, MeOH-D4, 400 Hz) 10.02 (s, 1H), 8.50-8.56 (m, 2H), 8.23-8.27 (m, 2H), 8.12 (d, 1H, J=2.48 Hz), 7.92-8.02 (m, 5H), 2.01 (s, 3H), 1.85 (s, 6H).

### Example 12

### N-(5-(1-(4-(2-cyanopropan-2-yl)phenyl)-3H-pyrazolo[3,4-c]quinolin-8-yl)pyridine-3-yl)methanesulfonamide (1265)

46% yield. MS (m/z) (M⁺+H): 483. ¹H-NMR: (δ, ppm, MeOH-D4, 400 Hz) 10.06 (s, 1H), 8.98 (s, 1H), 8.78 (s, 1H), 8.69 (d, 2H), 8.58-8.56 (d, 1H), 8.37-8.39 (d, 1H), 8.04-8.02 (d, 2H), 7.94-7.92 (d, 2H), 3.30 (s, 3H), 1.85(s, 6H).

### Example 13

### N-(5-(1-(4-(2-cyanopropan-2-yl)phenyl)-3H-pyrazolo[3,4-c]quinolin-8-yl)pyridine-3-yl)pivalamide (1266)

57% yield. MS (m/z) (M⁺+H): 489. ¹H-NMR: (δ, ppm, DMSO-d6, 400 Hz) 9.58 (s, 1H), 9.24 (s, 1H), 8.81(s, 1H), 8.49 (s, 1H), 8.45-8.42 (d, 2H), 8.17-8.15(d, 1H), 7.94-7.92(d, 2H), 7.82-7.80 (d, 1H), 7.76-7.74 (d, 2H), 1.75(s, 6H), 1.27 (s, 9H).

### Example 14

### N-(5-(1-(4-(2-cyanopropan-2-yl)phenyl)-3H-pyrazolo[3,4-c]quinolin-8-yl)pyridine-3-yl)cyclopropanecarboxamide cyclopropanecarboxylate (1267)

22% yield. MS (m/z) (M⁺+H): 472 as free base. ¹H-NMR: (δ, ppm, MeOH-D4, 400 Hz) 10.06 (s, 1H), 9.31 (s, 1H), 9.02 (s, 1H), 8.90 (s, 1H), 8.67 (s, 1H), 8.55 (d, 1H, J=8.52 Hz), 8.33 (d, 1H, J=8.24 Hz), 8.04-7.95 (m, 4H), 3.22-3.19 (m, 1H), 2.02-2.00 (m, 1H), 1.85 (s, 3H), 1.33-1.28 (m, 4H), 1.09-1.01 (m, 4H).

II-5. Method 5 Aryl Halide 23 Coupling with Amines followed by Cyclization

Palladium mediated ammination of aryl halide 23 with amines provided 44. The nitro-group in 44 was reduced to aryl amine (45), followed by acylation (46), ring closure (47), and de-acylation to afford the final compounds (48). The reaction conditions and yields were summarized in the following Table. A bi-product (Entry 5) from the coupling reaction was isolated and was also listed in the Table. The final compounds were summarized in Table II-5 (Example 5).

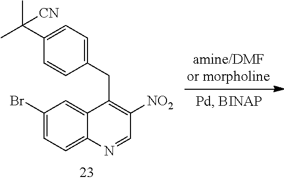

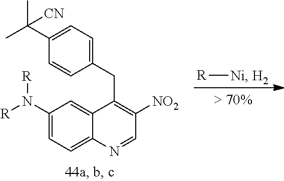

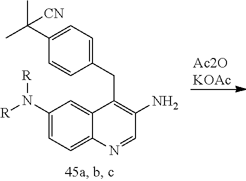

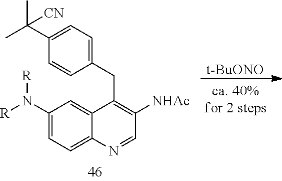

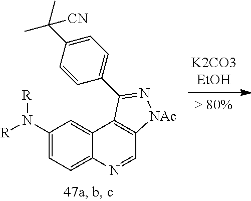

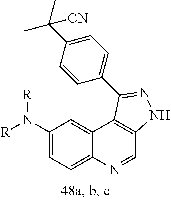

| Entry | NRR | Yield (%) |
|---|---|---|
| 1 | 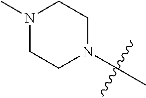 | 30%*61%*42%*57%¹ |
| 2 | 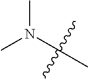 | 50%*65%*50%*80%¹ |
| 3 | 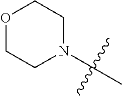 | 50%*78%*64%*86%¹ |
| 4 | 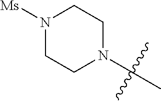 | 67%*65%*37%*80%¹ |
| 5 | 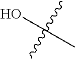 | 20%² |

Note:
1) yields of coupling, reduction, diazotization/cyclization and de-acetylation;
2) yield of product in Suzuki Coupling reaction.

TABLE II-5

| Cpd | | | | MS | |
|---|---|---|---|---|---|
| No | Ex | Structure | MF/MW | (M⁺ + H): | IUPAC |
| 1268 | 1 | 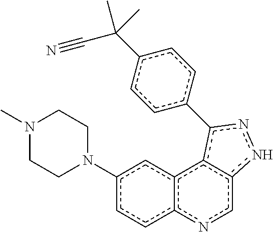 | C25H26N6/410.5 | 411 | 2-methyl-2-(4-(8-(4- methylpiperazin-1-yl)-3H- pyrazolo[3,4-c]quinolin-1- yl)phenyl)propanenitrile |
| 1269 | 2 | 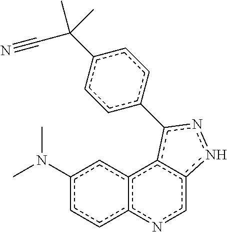 | C22H21N5/355.4 | 356 | 2-(4-(8-(dimethylamino)- 3H-pyrazolo[3,4- c]quinolin-1-yl)phenyl)-2- methylpropanenitrile |
| 1270 | 3 | 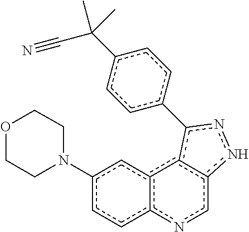 | C24H23N5O/397.5 | 398 | 2-methyl-2-(4-(8- morpholino-3H- pyrazolo[3,4-c]quinolin-1- yl)phenyl)propanenitrile |
| 1271 | 4 | 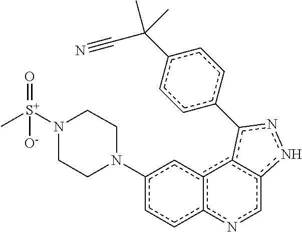 | C25H26N6O2S/474.6 | 475. | 2-methyl-2-(4-(8-(4- (methylsulfonyl)piperazin- 1-yl)-3H-pyrazolo[3,4- c]quinolin-1- yl)phenyl)propanenitrile |
| 1272 | 5 | 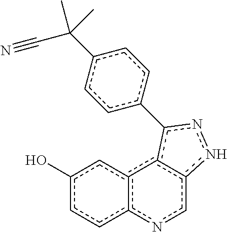 | C20H16N4O/328.4 | 329 | 2-(4-(8-hydroxy-3H- pyrazolo[3,4-c]quinolin-1- yl)phenyl)-2- methylpropanenitrile |

Synthetic Procedures for Preparing Compounds in Table II-5

### Example 1

### 2-methyl-2-(4-(8-(4-methylpiperazin-1-yl)-3H-pyrazolo[3,4-c]quinolin-1-yl)phenyl)propanenitrile (1268)

To a solution of 23, 2-(4-((6-bromo-3-nitroquinolin-4-yl)methyl)phenyl)-2-methyl propanenitrile (86 mg, 0.21 mmol) in DMF (5 mL) was added 1-methylpiperazine (100 mg, 1 mmol), Cs₂CO₃ (326 mg, 1 mmol), BINAP (12 mg, 0.02 mmol) and Pd₂(dba)₃ (18 mg, 0.02 mmol). The reaction mixture was stirred for 15 h at 14° C. The reaction mixture was diluted with water (10 mL) and extracted with DCM (3×20 mL). The prganic phase was combined, washed with brine, dried over Na₂SO₄, and filtered. The filtrate was concentrated. The resulting residue was purified by column chromatography (DCM:Methanol 80:1 to 60:1) to give 44a, 2-methyl-2-(4-((6-(4-methylpiperazin-1-yl)-3-nitroquinolin-4-yl)methyl)phenyl)propanenitrile (30 mg, 32%) as a light yellow solid and byproduct 44b, 2-(4-((6-(dimethylamino)-3-nitroquinolin-4-yl)methyl)phenyl)-2-methylpropanenitrile (38 mg, 50%) as a light yellow solid.

To a solution of 44a (30 mg, 0.07 mmol) in THF (30 mL) was added R—Ni (10 mg) at 25° C. and was hydrogenated with 1 atm hydrogen. The reaction mixture was stirred overnight. The solid was removed by filtration. The filtrate was concentrated to give crude 45a, 2-methyl-2-(4-((6-(4-methylpiperazin-1-yl)-3-aminoquinolin-4-yl)methyl) phenyl)propanenitrile (17 mg, 61%) as a light yellow solid.

To a solution of 45a (17 mg, 0.04 mmol) in toluene (50 mL) was added KOAc (30 mg, 0.27 mmol) and acetic anhydride (21 mg, 0.2 mmol). The reaction was monitored by TLC for the disappearance of starting material. To the reaction mixture was charged isoamylnitrite (5 mg, 0.05 mmol). The resulting mixture was heated to 80° C. and stirred for 18 h. The solvent was concentrated and the residue was purified by silica gel column chromatography (MeOH:DCM1:100 to 1:50) to give 47a, 2-(4-(3-acetyl-8-(4-methyl piperazin-1-yl)-3H-pyrazolo[3,4-c]quinolin-1-yl)phenyl)-2-methylpropanenitrile (8 mg, 42%) as a light yellow solid. To a solution of 47a (8 mg, 0.018 mmol) in MeOH (2 mL) was added K₂CO₃(1.4 mg, 0.01 mmol). The reaction mixture was stirred overnight, and concentrated to dryness. The residue was purified by silica gel column chromatography (MeOH:DCM1:80 to 1:30) to give 2-methyl-2-(4-(8-(4-methylpiperazin-1-yl)-3H-pyrazolo[3,4-c]quinolin-1-yl)phenyl)propanenitrile (48a). 57%. MS (m/z) (M⁺+H): 411. ¹H-NMR: (δ, ppm, MeOH-D4, 400MHz): 9.61 (s, 1H), 8.18 (d, 1H, J=9.17 Hz), 7.93-7.88 (m, 4H), 7.77-7.74 (m, 1H), 7.53 (d, 1H, J=2.57 Hz), 3.93 (d, 1H, J=12.46 Hz), 3.61 (d, 1H, J=10.99 Hz), 3.26-3.19 (m, 4H), 2.97 (s, 3H), 1.83 (s, 6H).

### Example 2

### 2-(4-(8-(dimethylamino)-3H-pyrazolo[3,4-c]quinolin-1-yl)phenyl)-2-methylpropanenitrile (1269)

To a solution of 44b (38 mg, 0.10 mmol) in THF (30 mL) was added R—Ni (10 mg) at 25° C. and was hydrogenated with 1 atm hydrogen. The reaction mixture was stirred overnight. The solid was removed by filtration, filtrated concentrated to give crude 45b, 2-(4-((6-(dimethylamino)-3-aminoquinolin-4-yl)methyl)phenyl)-2-methyl propanenitrile, (22 mg, 65%) as a light yellow solid.

To a solution of 45b (22 mg, 0.065 mmol) in toluene (50 mL) was added KOAc (30 mg, 0.27 mmol) and acetic anhydride (21 mg, 0.2 mmol). The reaction was monitored by TLC for the disappearance of starting material. To the reaction mixture was charged isoamylnitrite (7 mg, 0.07 mol). The resulting mixture was heated to 80° C. and stirred for 18 h. The solvent was concentrated and the residue was chromatographied (silica gel column, MeOH:DCM1:100 to 1:50) to give 47b, 2-(4-(3-acetyl-8-(dimethyl amino)-3H-pyrazolo[3,4-c]quinolin-1-yl)phenyl)-2-methylpropanenitrile, (15 mg, 50%) as a light yellow solid.

To a solution of 47b (15 mg, 0.038 mmol) in MeOH (2 mL) was added K₂CO₃ (2.8 mg, 0.02 mmol). The reaction mixture was stirred overnight, the solvent was concentrated and purified (MeOH:DCM1:80 to 1:30) to give 2-(4-(8-(dimethyl amino)-3H-pyrazolo[3,4-c]quinolin-1-yl)phenyl)-2-methylpropanenitrile (48b) (10 mg, 80%) as a yellow solid. MS (m/z) (M⁺+H): 356. ¹H-NMR (δ, ppm, DMSO-d6, 400MHz): 14.10 (s, 1H), 8.93 (s, 1H), 7.90 (d, 1H, J=9.20 Hz), 7.79 (d, 2H, J=8.00 Hz), 7.71 (d, 2H, J=7.60 Hz), 7.14 (d, 1H, J=8.00 Hz), 7.01(s, 1H), 2.83 (s, 6H), 1.74 (s, 6H).

### Example 3

### 2-methyl-2-(4-(8-morpholino-3H-pyrazolo[3,4-c]quinolin-1-yl)phenyl)propanenitrile (1270)

To a solution of 23 (86 mg, 0.21 mmol) in morpholine (2 mL) was added Cs₂CO₃ (326 mg, 1 mmol), BINAP (12 mg, 0.02 mmol) and Pd₂(dba)₃ (18 mg, 0.02 mmol). The reaction mixture was stirred for 15 h at 140° C. The reaction mixture was diluted with water (10 mL) and extracted with DCM (3×20 mL). The organic phases were combined, washed with brine, dried over Na₂SO₄, and filtered. The filtrate was concentrated. The resulting residue was purified by column chromatography (DCM:Methanol 80:1 to 60:1) to give 44c, 2-methyl-2-(4-((6-morpholino-3-nitroquinolin-4-yl)methyl)phenyl)propanenitrile (42 mg, 50%) as a light yellow solid.

To a solution of 44c (42 mg, 0.1 mmol) in THF (10 mL) was added R—Ni (0.1 g) at 25° C. and was hydrogenated with 1 atm hydrogen. The reaction mixture was stirred overnight. The solid was removed by filtration. The filtrate was concentrated to give crude 45c, 2-methyl-2-(4-((6-morpholino-3-aminoquinolin-4-yl)methyl)phenyl)propanenitrile (30 mg, 78%) as a light yellow solid.

To a solution of 45c (30 mg, 0.08 mmol) in toluene (50 mL) was added KOAc (30 mg, 0.3 mmol) and acetic anhydride (21 mg, 0.2 mmol). The reaction was monitored by TLC for the consumption of starting material. To the reaction mixture was charged isoamylnitrite (5 mg, 0.05 mol). The resulting mixture was heated to 80° C. and stirred for 18 h. The solvent was evaporated off, and the residue was purified (silica gel column, MeOH:DCM1:100 to 1:50) to give 47c, 2-(4-(3-acetyl-8-morpholino-3H-pyrazolo[3,4-c]quinolin-1-yl)phenyl)-2-methylpropanenitrile (22 mg, 64%) as a light yellow solid.

To a solution of 47c (22 mg, 0.05 mmol) in MeOH (2 mL) was added K₂CO₃ (1.4 mg, 0.01 mmol). The reaction mixture was stirred overnight. The solvent was concentrated and the residue was purified (MeOH:DCM1:80 to 1:30) to give 2-methyl-2-(4-(8-morpholino-3H-pyrazolo[3,4-c]quinolin-1-yl)phenyl)propanenitrile (48c) (17 mg, 86%). MS (m/z) (M⁺+H): 398. ¹H-NMR: (δ, ppm, MeOH-D4, 400 Hz) 9.51(s,1H), 8.13 (d, 1H, J=9.60 hz), 7.83 (bs, 4H), 7.68 (d, 1H, J=9.60 hz), 7.41 (s, 1H), 3.79 (bs, 4H), 3.20 (bs, 4H), 1.83 (s, 6H).

### Example 4

### 2-methyl-2-(4-(8-(4-(methylsulfonyl)piperazin-1-yl)-3H-pyrazolo[3,4-c]quinolin-1-yl)phenyl)propanenitrile (1271

To a solution of 23, 2-(4-((6-bromo-3-nitroquinolin-4-yl)methyl)phenyl)-2-methyl propanenitrile (162 mg, 0.40 mmol) in DMF (5 mL) was added 1-(methylsulfonyl) piperazine (164 mg, 1 mmol), Cs₂CO₃ (326 mg, 1 mmol), BINAP (12 mg, 0.02 mmol) and Pd₂(dba)₃ (18 mg, 0.02 mmol). The reaction mixture was stirred for 15 h at 140° C. The reaction mixture was diluted with water (10 mL) and extracted with DCM (3×20 mL). The organic phases were combined, washed with brine, dried over Na₂SO₄, and filtered. The filtrate was concentrated. The resulting residue was purified by column chromatography (DCM:Methanol 80:1 to 60:1) to give 44d, 2-methyl-2-(4-((6-(4-methylsulfonylpiperazin-1-yl)-3-nitroquinolin-4-yl)methyl)phenyl)propanenitrile (50 mg, 25%) as a light yellow solid.

To a solution of 44d (50 mg, 0.10 mmol) in THF (30 mL) was added R—Ni (20 mg) at 25° C. and was hydrogenated with 1 atm hydrogen. The reaction mixture was stirred overnight. The solid was removed by filtration. The filtrate was concentrated to give crude 45d, 2-(4-((3-amino-6-(4-methylsulfonylpiperazin-1-yl)quinolin-4-yl)methyl)phenyl)-2-methylpropanenitrile (30 mg, 65%) as a light yellow solid.

To a solution of 45d (30 mg, 0.065 mmol) in toluene (50 mL) was added KOAc (30 mg, 0.3 mmol) and acetic anhydride (21 mg, 0.2 mmol). The reaction was monitored by TLC for the comsuption of starting material. To the reaction mixture was charged isoamylnitrite (7 mg, 0.07 mol). The resulting mixture was heated to 80° C. and stirred for 18 h. The solvent was concentrated and the residue was purified (MeOH:DCM1:100 to 1:50) to give 47d, 2-(4-(3-acetyl-8-(4-methylsulfonyl piperazin-1-yl)-3H-pyrazolo[3,4-c]quinolin-1-yl)phenyl)-2-methylpropanenitrile (12 mg, 37%) as a light yellow solid.

To a solution of 47d (12 mg, 0.023 mmol) in MeOH (2 mL) was added K₂CO₃ (2.8 mg, 0.02 mmol). The reaction mixture was stirred overnight. The solvent was concentrated and the residue was purified (silica gel column, MeOH:DCM1:80 to 1:30) to give 2-methyl-2-(4-(8-(4-(methylsulfonyl)piperazin-1-yl)-3H-pyrazolo[3,4-c]quinolin-1-yl)phenyl)propanenitrile (48d) (10 mg, 80%) as a yellow solid. MS (m/z) (M⁺+H): 475. ¹H-NMR (δ, ppm, DMSO-d6, 400 MHz): 9.52 (s, 1H), 8.12 (d, 1H, J=8.80 Hz), 7.87 (bs, 4H), 7.69 (d, 1H, J=11.60 Hz), 7.43 (d, 1H, J=2.40 Hz), 3.35-3.30 (m, 8H), 2.88 (s, 3H), 1.86 (s, 6H).

### Example 5

### 2-(4-(8-hydroxy-3H-pyrazolo[3,4-c]quinolin-1-yl)phenyl)-2-methylpropanenitrile (1272)

was isolated as a byproduct in Suzuki coupling of boronate ester of 33 with arylbromides in ca. 20%. MS (m/z) (M⁺+H): 329. ¹H-NMR: (δ, ppm, MeOH-D4, 400 Hz) 8.97 (s,1H), 7.97 (d, 1H), 7.83 (m, 4H), 7.50 (d, 1H), 7.15 (m, 1H), 1.84 (s, 6H).

III. Preparation of of Intermediates and Targets (Scaffold J=)

Pyrazolo[3,4-c]quinoline derivatives with variation on 2- or 3-position is classified as Scaffold J=. Preparation of key intermediates for N2- and N3-substituted pyrazolo[3,4-c]quinoline derivatives is shown in the following Scheme. The structures of each pair of region isomers (25 vs. 28, 26 vs. 29, 27 vs. 30) were confirmed by NOE. The final compounds of N2- and N3-substituted pyrazolo[3,4-c]quinoline derivatives (52) were either prepared by Pd-coupling reactions of aryl halides (25-30) with pyridine boronic acid, or prepared from N-alkylation of pyrazole-NH with alkyl halides. All final compounds of Scaffold J=were summarized in Table II-5.

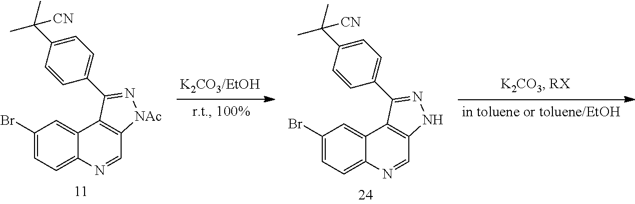

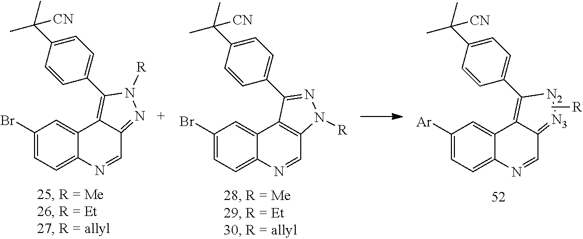

| Entry | Structure | Method | Yield (%) |
|---|---|---|---|
| 1 | 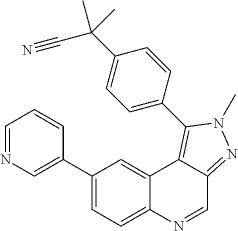 | Pd(PPh₃)_{4,} DMF/H₂O, microwave, 100- 120°C., Na₂CO₃ | 40% |
| 2 | 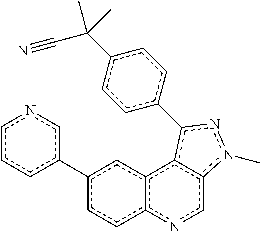 | Pd(PPh₃)_{4,} DMF/H₂O, microwave, 100- 120°C., Na₂CO₃ | 68% |
| 3 | 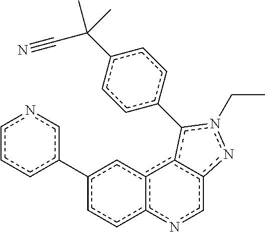 | Pd(PPh₃)_{4,} DMF/H₂O, microwave, 100- 120°C., Na₂CO₃ | 71% |
| 4 | 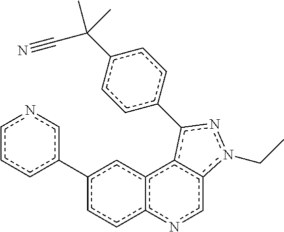 | Pd(PPh₃)_{4,} DMF/H₂O, microwave, 100- 120°C., Na₂CO₃ | 70% |
| 5 | 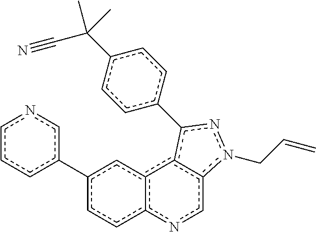 | Pd(PPh₃)_{4,} DMF/H₂O, microwave, 100- 120°C., Na₂CO₃ | 47% |
| 6 | 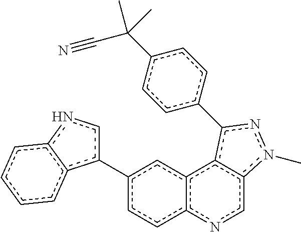 | Pd(PPh₃)_{4,} DMF/H₂O, microwave, 100- 120°C., Na₂CO₃ | 68% |
| 7 | 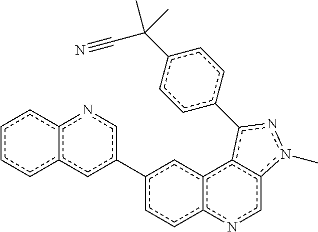 | Pd(PPh₃)_{4,} DMF/H₂O, microwave, 100- 120°C., Na₂CO₃ | 45% |
| 8 | 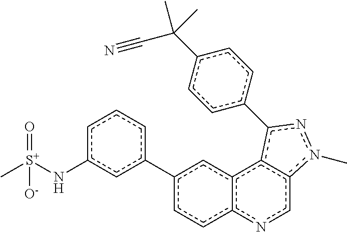 | Pd(PPh₃)_{4,} DMF/H₂O, microwave, 100- 120°C., Na₂CO₃ | 80% |
| 9 | 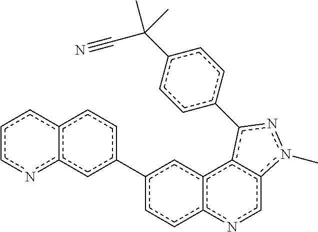 | Pd(PPh₃)_{4,} DMF/H₂O, microwave, 100- 120°C., Na₂CO₃ | 38% |
| 10 | 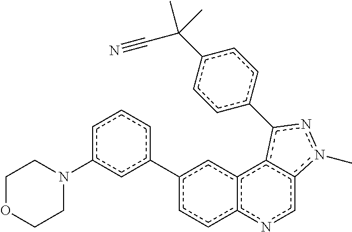 | Pd(PPh₃)_{4,} DMF/H₂O, microwave, 100- 120°C., Na₂CO₃ | 63% |
| 11 | 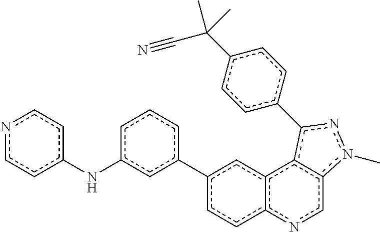 | Pd(PPh₃)_{4,} DMF/H₂O, microwave, 100- 120°C., Na₂CO₃ | 44% |
| 12 | 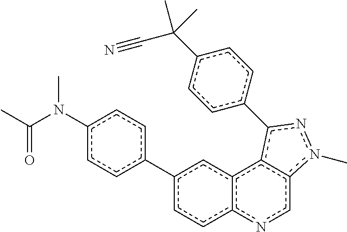 | Pd(PPh₃)_{4,} DMF/H₂O, microwave, 100- 120°C., Na₂CO₃ | 42% |
| 13 | 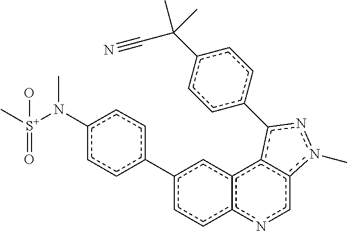 | Pd(PPh₃)_{4,} DMF/H₂O, microwave, 100- 120°C., Na₂CO₃ | 47% |
| 14 | 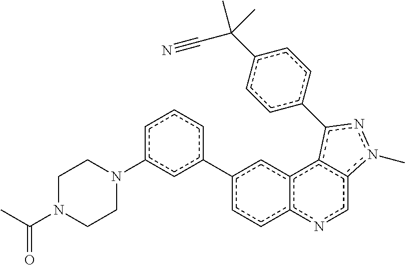 | Pd(PPh₃)_{4,} DMF/H₂O, microwave, 100- 120°C., Na₂CO₃ | 46% |
| 15 | 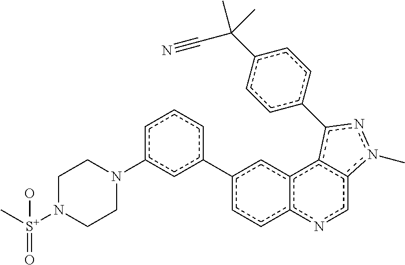 | Pd(PPh₃)_{4,} DMF/H₂O, microwave, 100- 120°C., Na₂CO₃ | 40% |
| 16 | 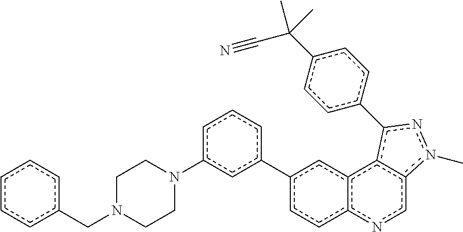 | Pd(PPh₃)_{4,} DMF/H₂O, microwave, 100- 120°C., Na₂CO₃ | 53% |
| 17 | 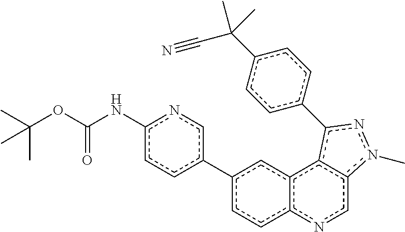 | Pd(PPh₃)_{4,} DMF/H₂O, microwave, 100- 120°C., Na₂CO₃ | 50% |
| 18 | 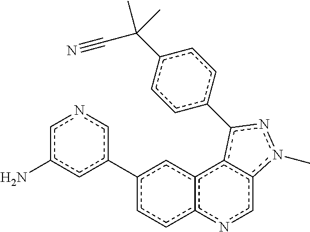 | HCl | 90% |
| 19 | 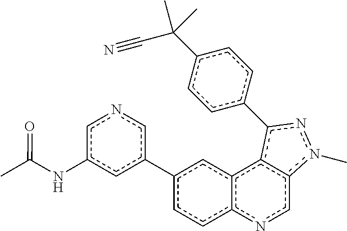 | AcCl/NEt₃ | 88% |
| 20 | 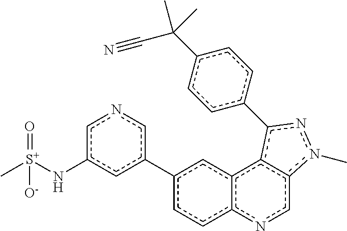 | MsCl/NEt₃ | 70% |
| 21 | 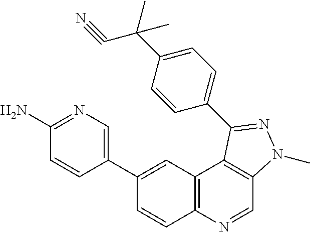 | HCl | 70% |
| 22 | 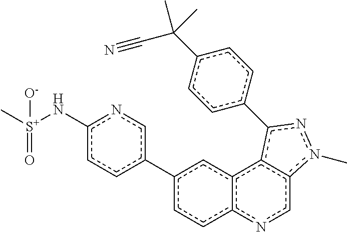 | AcCl/NEt₃ | 70% |
| 23 | 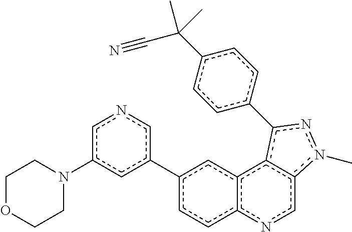 | MsCl/NEt₃ | 70% |
| 24 | 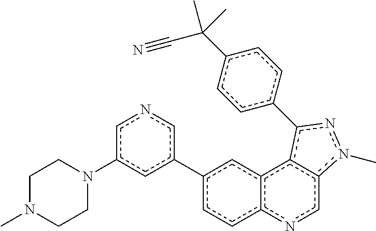 | Pd(PPh₃)_{4,} DMF/H₂O, reflux, 12 hrs K₂CO₃ | 27% |
| 25 | 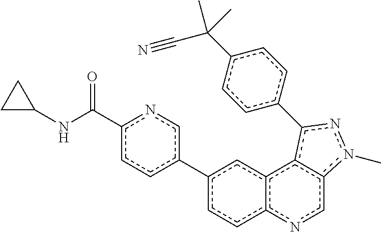 | Pd(PPh₃)_{4,} DMF/H₂O, reflux, 12 hrs K₂CO₃ | 30% |
| 26 | 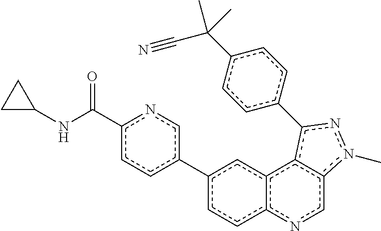 | Pd(PPh₃)_{4,} DMF/H₂O, reflux, 12 hrs K₂CO₃ | 70% |
| 27 | 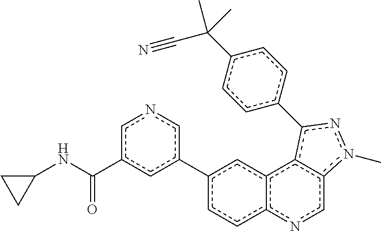 | Pd(PPh₃)_{4,} DMF/H₂O, reflux, 12 hrs K₂CO₃ | 30% |
| 28 | 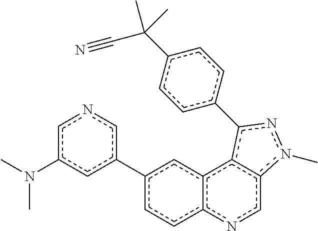 | Pd(PPh₃)_{4,} DMF/H₂O, reflux, 12 hrs K₂CO₃ | 38% |
| 29 | 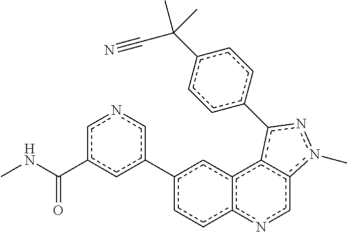 | Pd(PPh₃)_{4,} DMF/H₂O, reflux, 12 hrs K₂CO₃ | 37% |
| 30 | 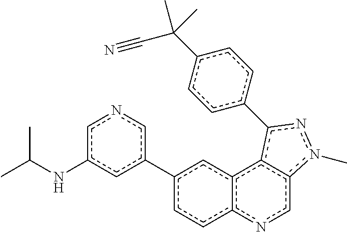 | Pd(PPh₃)_{4,} DMF/H₂O, reflux, 12 hrs K₂CO₃ | 26% |
| 31 | 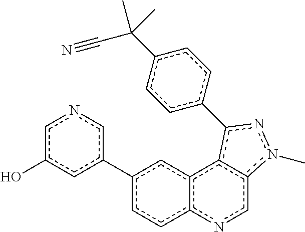 | Pd(PPh₃)_{4,} DMF/H₂O, reflux, 12 hrs K₂CO₃ | 12% |
| 32 | 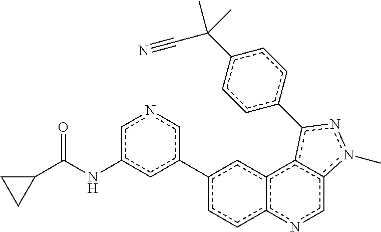 | Pd(PPh₃)_{4,} DMF/H₂O, reflux, 12 hrs K₂CO₃ | 33% |
| 33 | 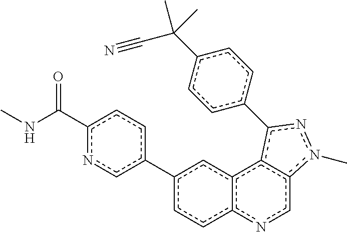 | Pd(PPh₃)_{4,} DMF/H₂O, reflux, 12 hrs K₂CO₃ | 56% |

TABLE III

| Cpd | | | | MS | |
|---|---|---|---|---|---|
| No | Ex | Structure | MF/MW | (M⁺ + H) | IUPAC |
| 1273 | 1 | 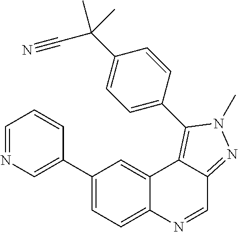 | C26H21N5/ 403.5 | 404 | 2-methyl-2-(4-(2-methyl-8-(pyridin- 3-yl)-2H-pyrazolo[3,4-c]quinolin-1- yl)phenyl)propanenitrile |
| 1274 | 2 | 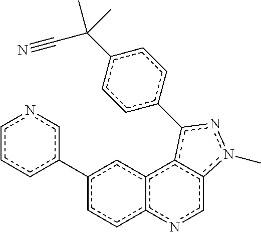 | C26H21N5/ 403.5 | 404 | 2-methyl-2-(4-(3-methyl-8-(pyridin- 3-yl)-3H-pyrazolo[3,4-c]quinolin-1- yl)phenyl)propanenitrile |
| 1275 | 3 | 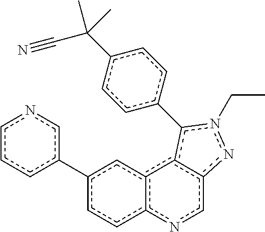 | C27H23N5/ 417.5 | 418 | 2-(4-(2-ethyl-8-(pyridin-3-yl)-2H- pyrazolo[3,4-c]quinolin-1- yl)phenyl)-2-methylpropanenitrile |
| 1276 | 4 | 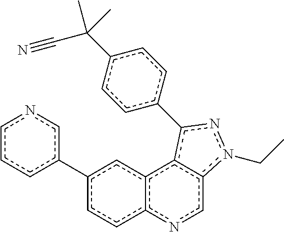 | C27H23N5/ 417.5 | 418 | 2-(4-(3-ethyl-8-(pyridin-3-yl)-3H- pyrazolo[3,4-c]quinolin-1- yl)phenyl)-2-methylpropanenitrile |
| 1277 | 5 | 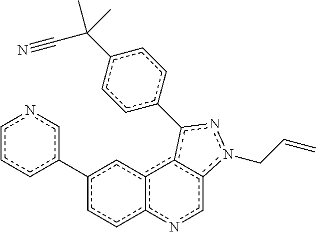 | C28H23N5/ 429.5 | 430 | 2-(4-(3-allyl-8-(pyridin-3-yl)-3H- pyrazolo[3,4-c]quinolin-1- yl)phenyl)-2-methylpropanenitrile |
| 1278 | 6 | 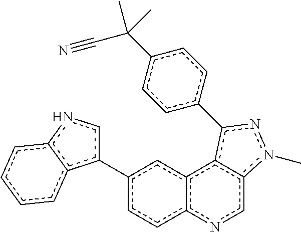 | C29H23N5/ 441.5 | 442 | 2-(4-(8-(1H-indol-3-yl)-3-methyl- 3H-pyrazolo[3,4-c]quinolin-1- yl)phenyl)-2-methylpropanenitrile |
| 1279 | 7 | 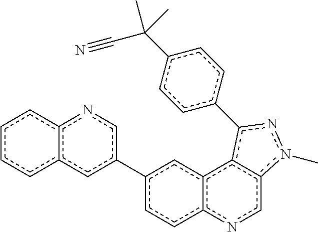 | C30H23N5/ 453.5 | 454.3 | 2-methyl-2-(4-(3-methyl-8- (quinolin-3-yl)-3H-pyrazolo[3,4- c]quinolin-1- yl)phenyl)propanenitrile |
| 1280 | 8 | 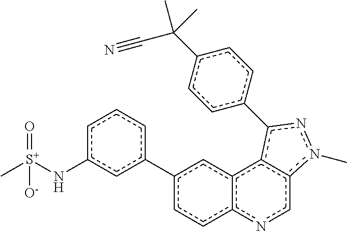 | C28H25N5O2S/ 495.6 | 496 | N-(3-(1-(4-(2-cyanopropan-2- yl)phenyl)-3-methyl-3H- pyrazolo[3,4-c]quinolin-8- yl)phenyl)methanesulfonamide |
| 1281 | 9 | 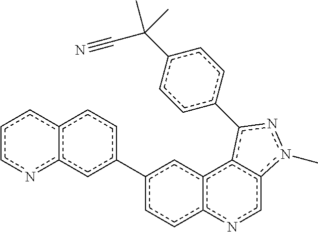 | C30H23N5/ 453.5 | 454 | 2-methyl-2-(4-(3-methyl-8- (quinolin-7-yl)-3H-pyrazolo[3,4- c]quinolin-1- yl)phenyl)propanenitrile |
| 1282 | 10 | 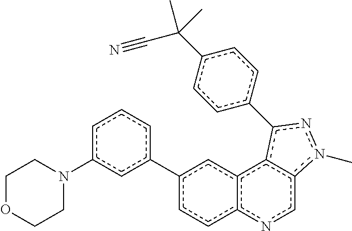 | C31H29N5O/ 487.6 | 488 | 2-methyl-2-(4-(3-methyl-8-(3- morpholinophenyl)-3H- pyrazolo[3,4-c]quinolin-1- yl)phenyl)propanenitrile |
| 1283 | 11 | 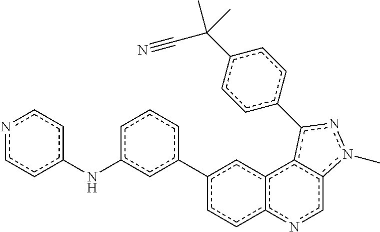 | C32H26N6/ 494.6 | 495 | 2-methyl-2-(4-(3-methyl-8-(3- (pyridin-4-ylamino)phenyl)-3H- pyrazolo[3,4-c]quinolin-1- yl)phenyl)propanenitrile |
| 1284 | 12 | 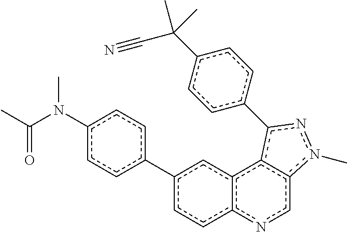 | C30H27N5O/ 473.6 | 474 | N-(4-(1-(4-(2-cyanopropan-2- yl)phenyl)-3-methyl-3H- pyrazolo[3,4-c]quinolin-8- yl)phenyl)-N-methylacetamide |
| 1285 | 13 | 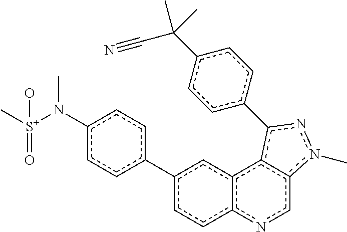 | C29H27N5O2S/ 509.6 | 510 | N-(4-(1-(4-(2-cyanopropan-2- yl)phenyl)-3-methyl-3H- pyrazolo[3,4-c]quinolin-8- yl)phenyl)-N- methylmethanesulfonamide |
| 1286 | 14 | 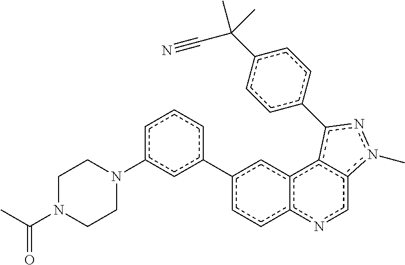 | C33H32N6O/ 528.6 | 529 | 2-(4-(8-(3-(4-acetylpiperazin-1- yl)phenyl)-3-methyl-3H- pyrazolo[3,4-c]quinolin-1- yl)phenyl)-2-methylpropanenitrile |
| 1287 | 15 | 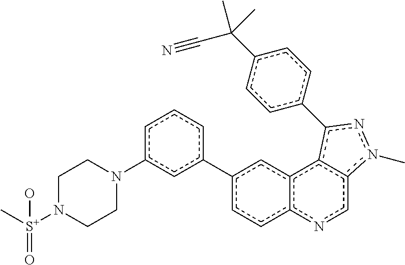 | C32H32N6O2S/ 564.7 | 565 | 2-methyl-2-(4-(3-methyl-8-(3-(4- (methylsulfonyl)piperazin-1- yl)phenyl)-3H-pyrazolo[3,4- c]quinolin-1- yl)phenyl)propanenitrile |
| 1288 | 16 | 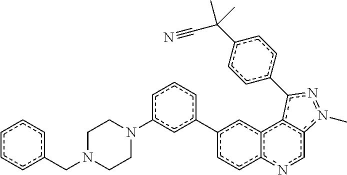 | C38H36N6/ 576.7 | 577 | 2-(4-(8-(3-(4-benzylpiperazin-1- yl)phenyl)-3-methyl-3H- pyrazolo[3,4-c]quinolin-1- yl)phenyl)-2-methylpropanenitrile |
| 1289 | 17 | 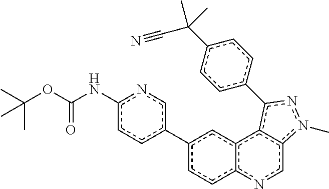 | C31H30N6O2/ 518.6 | 519 | tert-butyl 5-(1-(4-(2-cyanopropan-2- yl)phenyl)-3-methyl-3H- pyrazolo[3,4-c]quinolin-8- yl)pyridin-2-ylcarbamate |
| 1290 | 18 | 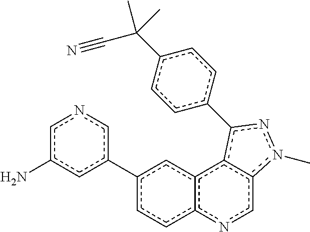 | C26H22N6/ 418.5 | 419 | 2-(4-(8-(5-aminopyridin-3-yl)-3- methyl-3H-pyrazolo[3,4-c]quinolin- 1-yl)phenyl)-2-methylpropanenitrile |
| 1291 | 19 | 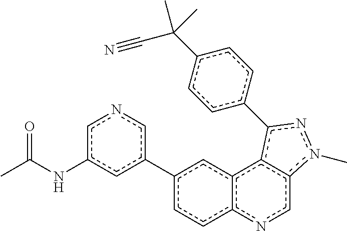 | C28H24N6O/ 460.5 | 461 | N-(5-(1-(4-(2-cyanopropan-2- yl)phenyl)-3-methyl-3H- pyrazolo[3,4-c]quinolin-8- yl)pyridin-3-yl)acetamide |
| 1292 | 20 | 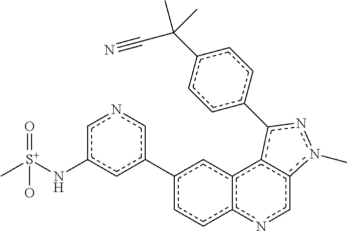 | C27H24N6O2S/ 496.6 | 497 | N-(5-(1-(4-(2-cyanopropan-2- yl)phenyl)-3-methyl-3H- pyrazolo[3,4-c]quinolin-8- yl)pyridin-3-yl)methanesulfonamide |
| 1293 | 21 | 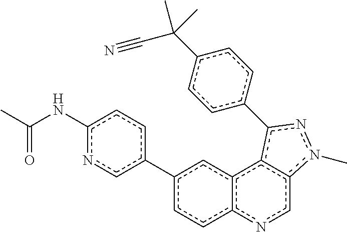 | C28H24N6O/ 460.5 | 461 | N-(5-(1-(4-(2-cyanopropan-2- yl)phenyl)-3-methyl-3H- pyrazolo[3,4-c]quinolin-8- yl)pyridin-2-yl)acetamide |
| 1294 | 22 | 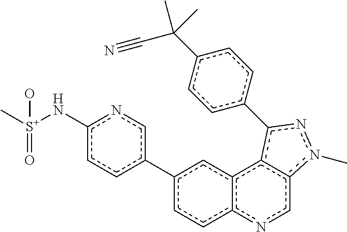 | C27H24N6O2S/ 496.6 | 497 | N-(5-(1-(4-(2-cyanopropan-2- yl)phenyl)-3-methyl-3H- pyrazolo[3,4-c]quinolin-8- yl)pyridin-2-yl)methanesulfonamide |
| 1295 | 23 | 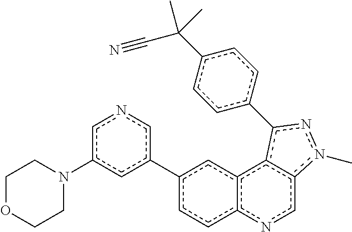 | C30H26N6O/ 488.6 | 489 | 2-methyl-2-(4-(3-methyl-8-(5- morpholinopyridin-3-yl)-3H- pyrazolo[3,4-c]quinolin-1- yl)phenyl)propanenitrile |
| 1296 | 24 | 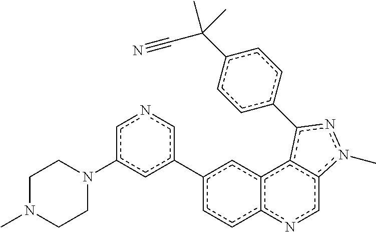 | C31H31N7/ 501.6 | 502 | 2-methyl-2-(4-(3-methyl-8-(5-(4- methylpiperazin-1-yl)pyridin-3-yl)- 3H-pyrazolo[3,4-c]quinolin-1- yl)phenyl)propanenitrile |
| 1297 | 25 | 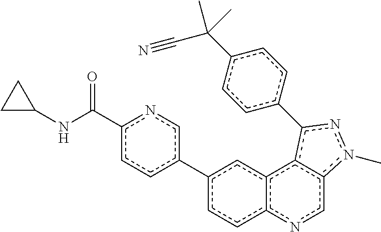 | C30H26N6O/ 486.6 | 487 | 5-(1-(4-(2-cyanopropan-2- yl)phenyl)-3-methyl-3H- pyrazolo[3,4-c]quinolin-8-yl)-N- cyclopropylpicolinamide |
| 1298 | 26 | 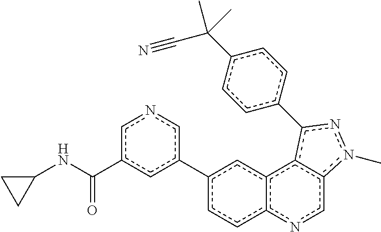 | C30H26N6O/ 486.6 | 487 | 5-(1-(4-(2-cyanopropan-2- yl)phenyl)-3-methyl-3H- pyrazolo[3,4-c]quinolin-8-yl)-N- cyclopropylnicotinamide |
| 1299 | 27 | 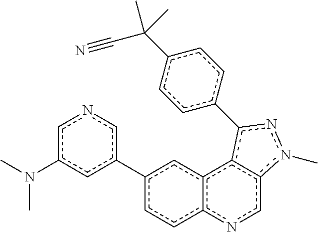 | C28H26N6/ 446.5 | 447 | 2-(4-(8-(5-(dimethylamino)pyridin- 3-yl)-3-methyl-3H-pyrazolo[3,4- c]quinolin-1-yl)phenyl)-2- methylpropanenitrile |
| 1300 | 28 | 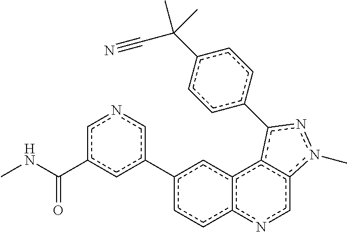 | C28H24N6O/ 460.5 | 461. | 5-(1-(4-(2-cyanopropan-2- yl)phenyl)-3-methyl-3H- pyrazolo[3,4-c]quinolin-8-yl)-N- methylnicotinamide |
| 1301 | 29 | 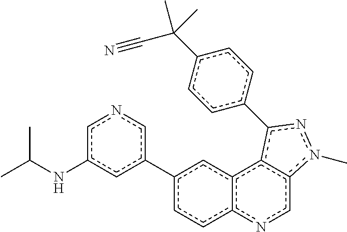 | C29H28N6/ 460.6 | 461 | 2-(4-(8-(5-(isopropylamino)pyridin- 3-yl)-3-methyl-3H-pyrazolo[3,4- c]quinolin-1-yl)phenyl)-2- methylpropanenitrile |
| 1302 | 30 | 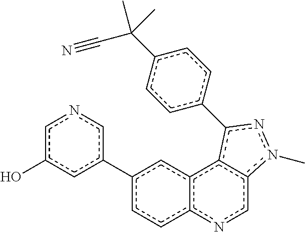 | C26H21N5O/ 419.5 | 420 | 2-(4-(8-(5-hydroxypyridin-3-yl)-3- methyl-3H-pyrazolo[3,4-c]quinolin- 1-yl)phenyl)-2-methylpropanenitrile |
| 1303 | 31 | 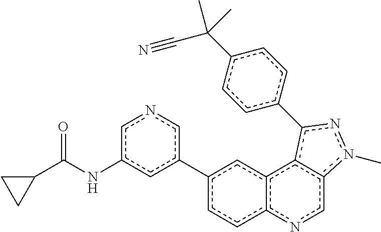 | C30H26N6O/ 486.6 | 486. | N-(5-(1-(4-(2-cyanopropan-2- yl)phenyl)-3-methyl-3H- pyrazolo[3,4-c]quinolin-8- yl)pyridin-3- yl)cyclopropanecarboxamide |
| 1304 | 32 | 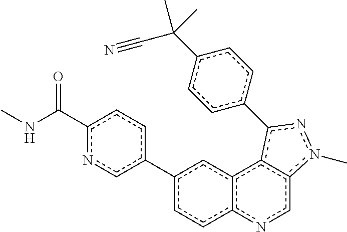 | C28H24N6O/ 460.5 | 461 | 5-(1-(4-(2-cyanopropan-2- yl)phenyl)-3-methyl-3H- pyrazolo[3,4-c]quinolin-8-yl)-N- methylpicolinamide |

Synthetic Procedures for Preparing the Compounds in Table III.

### 2-(4-(8-bromo-3H-pyrazolo[3,4-c]quinolin-1-yl)phenyl)-2-methylpropanenitrile (24)

To a solution of 11 (130 mg, 0.3 mmol) in EtOH (30 mL) was added K₂CO₃ (40 mg, 0.3 mmol). The reaction mixture was stirred overnight at room temperature. The solvent was concentrated and the residue was purified by solica gel column chromatography (EA:PE1:5 to 1:2) to give 24 (100 mg, 90%) as a light yellow solid. MS (m/z) (M⁺+H): 391, 393. ¹H-NMR: (δ, ppm, DMSO-d6, 400 MHz): 14.34 (s, 1H), 9.31 (s, 1H), 8.21-8.07 (m, 2H), 7.85-7.75 (m, 5H), 1.79 (s, 6H).

### 2-(4-(8-bromo-2-methyl-2H-pyrazolo[3,4-c]quinolin-1-yl)phenyl)-2-methylpropanenitrile (25) and 2-(4-(8-bromo-3-methyl-3H-pyrazolo[3,4-c]quinolin-1-yl)phenyl)-2-methylpropanenitrile (28)

To a solution of compound 24 (40 mg, 0.1 mmol) in EtOH/toluene (50 mL, v/v 1:1) was added K₂CO₃ (14 mg, 0.1 mmol). The reaction mixture was stirred overnight at room temperature. The solvent was concentrated and the residue was purified by column (EA:PE1:5 to 1:2) to give 28 (15 mg, 38%) and 25 (13 mg, 34%), respectively. ¹H-NMR (CDCl₃, 400 MHz, ppm) of 25, 69.29 (s, 1H), 7.99(d, 1H, J=8.8 Hz), 7.80(d, 2H), 7.70(s, 1H), 7.63-7.58(m, 3H), 4.08(s, 3H), 1.88(s, 6H). NOE-DIFF: irradiated at 4.33 ppm, resonance at 9.18 ppm (□-proton on quinoline), irradiated at 9.18 ppm, resonance at 4.33 ppm. ¹H-NMR (CDCl₃, 400 MHz, ppm) of 28, δ9.18(s, 1H), 8.35(s, 1H), 8.10(d, 1H, J=4.1 Hz), 7.82(d, 2H), 7.71-7.69(t, 3H), 4.33(s, 3H), 1.84(s, 6H). MS (m/z) (M⁺+H) for 25 and 28: 407, 405. NOE-DIFF: irradiated at 4.08 ppm, resonance at 7.58 ppm (phenyl proton), irradiated at 9.29 ppm, no resonance.

### 2-(4-(8-bromo-2-ethyl-2H-pyrazolo[3,4-c]quinolin-1-yl)phenyl)-2-methylpropanenitrile (26) and 2-(4-(8-bromo-3-ethyl-3H-pyrazolo[3,4-c]quinolin-1-yl)phenyl)-2-methylpropanenitrile (29)

To a solution of 24 (8 mg, 0.01 mmol) in EtOH/toluene (50 mL, v/v 1:4) was added EtBr (2.4 mg, 0.011 mmol) and K₂CO₃ (1.4 mg, 0.01 mmol). The reaction mixture was stirred overnight at 55° C. HPLC indicated that ratio of 26: 29 was 60:40. Pure 26 and 29 were obtained through flash chromatography with the yield of 46% and 32%, respectively. MS (m/z) (M⁺+H) for 26 and 29: 419, 421. ¹H-NMR for 26: 9.19 (s, 1H), 8.36 (d, 1H, J=2.01 Hz), 8.08 (d, 1H, J=8.68 Hz), 7.94-7.77 (m, 2H), 7.83-7.69 (m, 5H), 4.71-4.65 (m, 2H), 1.84 (s, 6H), 1.70-1.65 (m, 3H). NOE-DIFF of 26: irradiated at 9.31 ppm, no resonance was observed. ¹H-NMR for 29: 9.19 (s, 1H), 8.36(d, 1H, J=2.01), 8.08(d, 1H, J=8.68),7.94-7.77 (m, 2H), 7.83-7.69(m, 5H), 4.71-4.65(m, 2H), 1.84 (s, 6H),1.70-1.65(m, 3H). NOE-DIFF of 29: irradiated at 9.19 ppm, resonance appeared at 4.71-4.65 ppm.

### 2-(4-(3-allyl-8-bromo-3H-pyrazolo[3,4-c]quinolin-1-yl)phenyl)-2-methylpropanenitrile (30)

To a solution of 24 (80 mg, 0.2 mmol) in EtOH/toluene (50 mL, v/v 1:1) was added 3-bromoprop-1-ene (27 mg, 0.22 mmol) and K₂CO₃ (28 mg, 0.2 mmol). The reaction mixture was stirred overnight at room temperature, the solvent was concentrated. The residue was purified by silica gel column chromatography (EA:PE1:5) to give 30 (30 mg, 35%) as a light yellow solid. MS (m/z) (M⁺+H): 431, 433, ¹H-NMR: 9.18 (s, 1H), 8.34 (d, 1H, J=2.00 Hz), 8.08 (d, 1H, J=8.40 Hz), 7.83-7.69 (m, 5H), 6.18-6.10 (m, 1H), 5.37-5.24 (m, 4H), 1.85 (s, 6H). NOE-DIFF of 30: irradiated at 9.18 ppm, resonance appeared at 5.25 ppm.

### General Procedures for Preparing Examples 1-17 (Examples 1)

To a solution of 51, 2-(4-(8-bromo-2-methyl-2H-pyrazolo[3,4-c]quinolin-1-yl) phenyl)-2-methylpropanenitrile (scaffold J=1, 20 mg, 0.05 mmol) in DMF (2 mL) was added 3-pyridylboronic acid (112 mg, 1 mmol), 1M Na₂CO₃ (100 mg, 0.6 mmol, in 0.6 mL water) and Pd(PPh₃)₄ (6 mg, 0.1 eq). The reaction mixture was protected with N₂, and stirred under microwave for 15 min at 100° C. The mixture was diluted with water (10 mL) and extracted with DCM (3×20 mL). Organic layer was washed with brine, dried over Na₂SO₄, filtered, and concentrated. The residue was purified by column chromatography (DCM:Methanol 80:1 to 60:1) to give the target product (8 mg, 40%) as a light yellow solid.

### Example 1

### 2-methyl-2-(4-(2-methyl-8-(pyridin-3-yl)-2H-pyrazolo[3,4-c]quinolin-1-yl)phenyl)propanenitrile (1273)

40% yield. MS (m/z) (M⁺+H): 404. ¹H-NMR (ppm, DMSO-d6, 400 MHz): 9.29 (s, 1H), 8.94 (s, 1H), 8.68-8.55(t, 2H), 8.15-8.11 (d, 2H), 7.93 (d, 1H), 7.88 (m, 2H), 7.74 (s, 1H), 7.42 (s, 1H), 7.39 (s, 1H), 4.11 (s, 3H), 1.80 (s, 6H).

### Example 2

### 2-methyl-2-(4-(3-methyl-8-(pyridin-3-yl)-3H-pyrazolo [3,4-c]quinolin-1-yl)phenyl)propanenitrile (1274)

68% yield. MS (m/z) (M⁺+H): 404. ¹H-NMR: (δ, ppm, DMSO-d6, 400 Hz) 9.44 (s, 1H), 8.80 (s, 1H), 8.57 (d, 1H, J=4.30 Hz), 8.23 (s, 1H), 8.21 (s, 1H), 7.94-8.00 (m, 2H), 7.87 (d, 2H, J=8.61 Hz), 7.76 (d, 2H, J=8.21 Hz), 7.46-7.43 (m, 1H), 4.33 (s, 3H), 1.77 (s, 6H).

### Example 3

### 2-(4-(2-ethyl-8-(pyridin-3-yl)-2H-pyrazolo[3,4-c]quinolin-1-yl)phenyl)-2-methylpropanenitrile (1275)

71% yield. MS (m/z) (M⁺+H): 418. ¹H-NMR: (δ, ppm, DMSO-d6, 400 Hz) 9.32 (s, 1H), 8.64 (s, 1H), 8.54 (d, 1H, J=3 Hz), 8.11 (d, 1H, J=8.3 Hz), 7.95-7.80 (m, 5H), 7.63 (s, 1H), 7.42-7.39 (m, 1H), 4.38 (q, 2H, J=7.3 Hz), 1.81 (s, 6H), 1.46 (t, 3H).

### Example 4

### 2-(4-(3-ethyl-8-(pyridin-3-yl)-3H-pyrazolo[3,4-c]quinolin-1-yl)phenyl)-2-methylpropanenitrile (1276

70% yield. MS (m/z) (M⁺+H): 418. ¹H-NMR: (δ, ppm, DMSO-d6, 400 Hz) 9.49 (s, 1H), 8.79 (s, 1H), 8.56 (d, 1H, J=3 Hz), 8.22 (m, 1H), 7.99-7.74 (m, 5H), 7.63-7.42 (m, 3H), 4.73 (q, 2H, J=7.3 Hz), 1.77 (s, 6H), 1.55 (t, 3H).

### Example 5

### 2-(4-(3-allyl-8-(pyridin-3-yl)-3H-pyrazolo[3,4-c]quinolin-1-yl)phenyl)-2-methylpropanenitrile (1277)

47% yield. MS (m/z) (M⁺+H): 430. ¹H-NMR: (δ, ppm, DMSO-d6, 400 Hz) 9.47 (s, 1H), 8.81 (s, 1H), 8.56 (s, 1H, J=4.76 Hz), 8.27 (s, 1H), 8.24 (s, 1H), 7.97-8.03 (m, 2H), 7.89 (d, 2H, J=8.23 Hz), 7.77 (d, 2H, J=8.24 Hz), 7.44-7.47 (m, 1H), 6.13-6.20 (m, 1H), 5.37 (d, 2H, J=5.67 Hz), 5.21-5.27 (m, 2H), 1.76 (s, 6H).

### Example 6

### 2-(4-(8-(1H-indol-3-yl)-3-methyl-3H-pyrazolo[3,4-c]quinolin-1-yl)phenyl)-2-methylpropanenitrile (1278)

68% yield. MS (m/z) (M⁺+H): 442. ¹H-NMR: (δ, ppm, DMSO-d6, 400 Hz) 11.17 (s, 1H), 9.23 (s, 1H), 8.06 (d, 1H, J=8.22 Hz), 7.86-7.91 (m, 5H), 7.81 (s, 1H), 7.65 (s, 1H), 7.40 (s, 1H), 7.38 (dd, 1H, J=13.52 Hz, J=24.46 Hz), 7.21 (dd, 1H, J=11.56 Hz, J=27.16 Hz), 6.44 (s, 1H), 4.10 (s, 3H), 1.81 (s, 6H).

### Example 7

### 2-methyl-2-(4-(3-methyl-8-(quinolin-3-yl)-3H-pyrazolo[3,4-c]quinolin-1-yl)phenyl)propanenitrile (1279)

45% yield. MS (m/z) (M⁺+H): 454. ¹ H-NMR: (δ, ppm, DMSO-d6, 400 Hz) 9.32 (s, 1H), 9.03 (d, 1H, J=1.96 Hz), 8.41 (s, 1H), 8.18 (d, 1H, J=8.22 Hz), 8.09 (d, 1H, J=8.61 Hz), 8.01 (t, 2H, J=8.21 Hz), 7.94 (s, 1H), 7.91 (s, 1H), 7.76 (t, 1H, J=7.82 Hz), 7.63 (t, 1H, J=7.43 Hz), 4.12 (s, 3H), 1.79 (s, 6H).

### Example 8

### N-(3-(1-(4-(2-cyanopropan-2-yl)phenyl)-3-methyl-3H-pyrazolo[3,4-c]quinolin-8-yl)phenyl)methanesulfonamide (1280)

80% yield. MS (m/z) (M⁺+H): 496. ¹H-NMR: (δ, ppm, DMSO-d6, 400 Hz) 9.43 (s, 1H), 8.34 (d, 1H, J=1.86 Hz), 8.22 (d, 1H, J=8.61 Hz), 7.95 (dd, 1H, J=11.96Hz, J=26.79 Hz), 7.89 (d, 2H, J=8.21 Hz), 7.74 (d, 2H, J=8.22 Hz), 7.64 (s, 1H), 7.41-7.50 (m, 3H), 4.32 (s, 3H), 2.93 (s, 3H), 1.75 (s, 6H).

### Example 9

### 2-methyl-2-(4-(3-methyl-8-(quinolin-7-yl)-3H-pyrazolo[3,4-c]quinolin-1-yl)phenyl)propanenitrile (1281)

38% yield. MS (m/z) (M⁺+H): 454. ¹H-NMR: (δ, ppm, DMSO-d6+D2O, 400 Hz) 10.03 (d, 1H, J=2.74 Hz), 9.33 (d, 1H, J=1.95 Hz), 8.90 (s, 1H), 8.38 (d, 1H, J=9.0 Hz), 8.31 (d, 1H, J=8.60 Hz), 7.99-8.04 (m, 2H), 7.92 (s, 3H), 7.84 (t, 2H, J=8.22 Hz), 4.24 (s, 3H), 1.74 (s, 6H).

### Example 10

### 2-methyl-2-(4-(3-methyl-8-(3-morpholinophenyl)-3H-pyrazolo[3,4-c]quinolin-1-yl)phenyl)propanenitrile (1282)

63% yield. MS (m/z) (M⁺+H): 488. ¹H-NMR: (δ, ppm, DMSO-d6, 400 Hz) 9.44 (s, 1H), 8.30 (d, 1H, J=1.95 Hz), 8.22 (d, 1H, J=8.61 Hz), 7.96 (d, 1H, J=1.96 Hz), 7.93 (d, 1H, J=8.22 Hz), 7.76 (d, 2H, J=8.61 Hz), 7.57-7.65 (m, 2H), 7.29 (t, 1H, J=8.21 Hz), 7.17 (s, 1H), 6.97-7.02 (s, 1H), 4.35 (s, 3H), 3.79 (t, 4H, 2OCH₂, J=4.31 Hz), 3.17 (t, 4H, 2NCH₂, J=4.70 Hz), 1.79 (s, 6H).

### Example 11

### 2-methyl-2-(4-(3-methyl-8-(3-(pyridin-4-ylamino)phenyl)-3H-pyrazolo[3,4-c]quinolin-1-yl)phenyl)propanenitrile (1283)

44% yield. MS (m/z) (M⁺+H): 495. ¹H-NMR: (δ, ppm, DMSO-d6, 400 Hz) 9.46 (s, 1H), 8.95 (s, 1H), 8.32 (d, 1H, J=1.95 Hz), 8.21-8.26 (m, 3H), 7.96 (dd, 1H, J=15.05 Hz, J=22.3 Hz), 7.90 (d, 2H, J=8.41 Hz), 7.74 (d, 2H, J=8.41 Hz), 7.42 (t, 1H, J=1.83 Hz), 7.2-7.34 (m, 2H), 6.96 (d, 1H, J=6.45 Hz), 4.36 (s, 3H), 1.67 (s, 6H).

### Example 12

### N-(4-(1-(4-(2-cyanopropan-2-yl)phenyl)-3-methyl-3H-pyrazolo[3,4-c]quinolin-8-yl)phenyl)-N-methylacetamide (1284)

42% yield. MS (m/z) (M⁺+H): 474. ¹H-NMR: (δ, ppm, DMSO-d6, 400 Hz) 9.26 (s, 1H), 8.08 (d, 1H, J=8.41 Hz), 7.83-7.89 (m, 6H), 7.73 (s, 1H), 7.50 (d, 2H, J=8.06 Hz), 7.33 (d, 2H, J=8.06 Hz), 4.10 (s, 3H), 3.13 (s, 3H), 1.79 (s, 6H).

### Example 13

### N-(4-(1-(4-(2-cyanopropan-2-yl)phenyl)-3-methyl-3H-pyrazolo[3,4-c]quinolin-8-yl)phenyl)-N-methylmethanesulfonamide (1285)

47% yield. MS (m/z) (M⁺+H): 510. ¹H-NMR: (δ, ppm, DMSO-d6, 400 Hz) 9.26 (s, 1H), 8.08 (d, 1H, J=8.60 Hz), 7.83-7.88 (m, 4H), 7.70 (s, 1H), 7.47-7.60 (m, 2H), 7.47 (d, 2H, J=8.24 Hz), 7.40 (d, 2H, J=8.41 Hz), 4.10 (s, 3H), 3.21 (s, 3H), 2.92 (s, 3H), 1.80 (s, 6H).

### Example 14

### 2-(4-(8-(3-(4-acetylpiperazin-1-yl)phenyl)-3-methyl-3H-pyrazolo[3,4-c]quinolin-1-yl)phenyl)-2-methylpropanenitrile (1286)

46% yield. MS (m/z) (M⁺+H): 529. ¹H-NMR: (δ, ppm, DMSO-d6, 400 Hz) 9.28 (s, 1H), 8.10 (d, 1H, J=8.29 Hz), 7.86-7.90 (m, 5H), 7.78 (d, 1H, J=1.95 Hz), 7.24 (t, 1H, J=8.29 Hz), 7.08 (s, 1H), 6.97 (dd, 1H, J=11.95 Hz, J=26.60 Hz), 6.84 (d, 1H, J=7.80 Hz), 4.10 (s, 3H), 3.59-3.62 (m, 4H), 3.20 (t, 2H, J=4.88 Hz), 3.13 (t, 2H, J=4.87 Hz), 2.05 (s, 3H), 1.80 (s, 6H).

### Example 15

### 2-methyl-2-(4-(3-methyl-8-(3-(4-(methylsulfonyl)piperazin-1-yl)phenyl)-3H-pyrazolo[3,4-c]quinolin-1-yl)phenyl)propanenitrile (1287)

40% yield. MS (m/z) (M⁺+H): 565. ¹H-NMR: (δ, ppm, DMSO-d6, 400 Hz) 9.28 (s, 1H), 8.10 (d, 1H, J=8.78 Hz), 7.89 (d, 1H, J=1.95 Hz), 7.87 (s, 4H), 7.77 (d, 1H, J=1.95 Hz), 7.25 (t, 1H, J=8.29 Hz), 7.12 (s, 1H), 6.98 (dd, 1H, J=11.95 Hz, J=26.63 Hz), 6.84 (d, 1H, J=7.80 Hz), 4.10 (s, 3H), 3.28-3.33 (m, 8H), 2.94 (s, 3H), 1.85 (s, 6H).

### Example 16

### 2-(4-(8-(3-(4-benzylpiperazin-1-yl)phenyl)-3-methyl-3H-pyrazolo[3,4-c]quinolin-1-yl)phenyl)-2-methylpropanenitrile (1288)

53% yield. MS (m/z) (M⁺+H): 577. ¹H-NMR: (δ, ppm, DMSO-d6, 400 Hz) 10.09 (s, 1H), 8.54 (d, 1H, J=8.61 Hz), 8.17 (dd, 1H, J=11.95 Hz, J=27.46 Hz), 7.90 (s, 4H), 7.77 (d, 1H, J=1.95 Hz), 7.67 (d, 2H, J=9.25 Hz), 7.43 (t, 3H, J=3.13 Hz), 7.21-7.26 (m, 2H), 7.02-6.99 (dd, 1H, J=11.96 Hz, J=26.56 Hz), 6.75 (d, 1H, J=7.82 Hz), 4.21 (s, 3H), 3.88 (d, 2H, J=12.56 Hz), 3.28-3.35 (m, 4H), 1.78 (s, 6H).

### Example 17

### tert-butyl-5-(1-(4-(2-cyanopropan-2-yl)phenyl)-3-methyl-3H-pyrazolo[3,4-c quinolin-8-yl)pyridin-2-ylcarbamate (1289)

50% yield. MS (m/z) (M⁺+H): 519. ¹H-NMR: (δ, ppm, MeOH-D4+CDCl3, 400 Hz) 9.23 (d, 1H, J=6.65 Hz), 8.54 (d, 1H, J=1.96 Hz), 8.19-8.08 (m, 3H), 7.91-7.77 (m, 6H), 4.16 (s, 3H), 1.75 (s, 6H), 1.56 (s, 9H).

### Example 18

### 2-(4-(8-(5-aminopyridin-3-yl)-3-methyl-3H-pyrazolo[3,4-c]quinolin-1-yl)phenyl)-2-methylpropanenitrile (1290)

To a solution of 28, 2-(4-(8-bromo-2-methyl-2H-pyrazolo[3,4-c]quinolin-1-yl) phenyl)-2-methylpropanenitrile (scaffold J=1, 82 mg, 0.2 mmol) in DMF (10 mL) was added tert-butyl 5-(4,4,5,5-tetramethyl-1,3,2-dioxaborolan-2-yl)pyridin-3-ylcarbamate (64 mg, 0.2 mmol), Na₂CO₃ (80 mg, in 0.5 mL water) and Pd(PPh₃)₄ (11 mg, 0.01 mmol). The reaction mixture was stirred under microwave for 0.5 h at 120° C. The reaction mixture was diluted with water (200 mL) and extracted with DCM (3×250 mL). The organic layer was combined, washed with brine, dried over Na₂SO₄, and filtered. The filtrate was concentrated. The resulting residue was purified by column chromatography (DCM:Methanol 50:1 to 30:1) to give tert-Butyl 5-(1-(4-(2-cyanopropan-2-yl)phenyl)-3-methyl-3H-pyrazolo[3,4-c]quinolin-8-yl)pyridin-3-ylcarbamate as a yellow solid (54 mg, 52%). MS (m/z) (M⁺+H): 519. To a solution of tert-Butyl 5-(1-(4-(2-cyanopropan-2-yl)phenyl)-3-methyl-3H-pyrazolo[3,4-c]quinolin-8-yl)pyridin-3-ylcarbamate (104 mg, 0.2 mmol) in MeOH (10 mL) was added concentrated HCl (0.5 mL). The reaction mixture was stirred for 2 h at rt, then NaHCO₃ solid was added to adJ=ust pH 7.5. The mixture was filtered to remove solid. The filtrate was concentrated to give the target product (73 mg, 90%) as brown solid. MS (m/z) (M⁺+H): 419. ¹H-NMR: (δ, ppm, MeOH-D4, 400 Hz): 10.19 (s, 1H), 8.58-8.54 (m, 2H), 8.29-8.25 (m, 2H), 8.13 (d, 1H, J=2.55 Hz), 7.99 (d, 2H, J=8.21 Hz), 7.94-7.89 (m, 2H), 4.58 (s, 3H), 1.84 (s, 6H).

### Example 19

### N-(5-(1-(4-(2-cyanopropan-2-yl)phenyl)-3-methyl-3H-pyrazolo[3,4-c]quinolin-8-yl)□yridine-3-yl)acetamide (1291)

To a solution of Example 18 (21 mg, 0.05 mmol) in DCM (5 mL) was added Py (0.5 mL) and Ac₂O (10 mg, 0.1 mmol). The reaction mixture was stirred for 15 h at rt. The mixture was concentrated and purified by chromatography (MeOH:DCM1:20) to give (20 mg, 88%) as brown solid. MS (m/z) (M⁺+H): 461. ¹H-NMR: (δ, ppm, MeOH-D4, 300 Hz) 10.20 (s, 1H), 9.16 (s, 1H), 9.14 (s, 1H), 8.92 (s, 1H), 8.73 (s, 1H), 8.57 (d, 1H, J=8.71 Hz), 8.37 (d, 1H, J=6.76 Hz), 8.01 (d, 2H, J=8.24 Hz), 7.92 (d, 2H, J=8.09 Hz), 4.58 (s, 3H), 2.31 (s, 3H), 1.83 (s, 6H).

### Example 20

### N-(5-(1-(4-(2-cyanopropan-2-yl)phenyl)-3-methyl-3H-pyrazolo[3,4-c]quinolin-8-yl)pyridine-3-yl)methanesulfonamide (1292)

To a solution of 2-(4-(8-(5-aminopyridin-3-yl)-3-methyl-3H-pyrazolo[3,4-c]quinolin-1-yl)phenyl)-2-methylpropanenitrile (Example 18) (21 mg, 0.05 mmol) in DCM (5 mL) was added Py (0.5 mL) and MsCl (6 mg, 0.05 mmol). The reaction mixture was stirred for 2 h at rt. The mixture was concentrated and purified by chromatography ((MeOH:DCM1:20)) to give target product (17 mg, 70%) as brown solid. MS (m/z) (M⁺+H): 497. ¹H-NMR: (δ, ppm, MeOH-D4, 300 Hz) 10.20 (s, 1H), 8.96-8.90 (m, 2H), 8.74-8.56 (m, 3H), 8.38 (d, 1H, J=8.86 Hz), 8.18-8.16 (m, 1H), 8.00 (d, 2H, J=8.24 Hz), 7.88 (d, 2H, J=8.25 Hz), 4.58 (s, 3H), 3.28 (s, 3H), 1.84 (s, 6H).

### Example 20.5

### 2-(4-(8-(6-aminopyridin-3-yl)-3-methyl-3H-pyrazolo[3,4-c]quinolin-1-yl)phenyl)-2-methylpropanenitrile

Generic procedure to de-Boc is the same as for preparing Example 18. To a solution of compound Example 17 (63 mg, 0.12 mmol) in MeOH (10 mL) was added concentrated HCl (0.5 mL). The reaction mixture was stirred for 2 h at rt, then NaHCO₃ solid was added to adJ=ust PH 7.5. The mixture was filtrated and the filtrate was concentrated to give 21 (41 mg, 83%) as brown solid.

### Example 21

### N-(5-(1-(4-(2-cyanopropan-2-yl)phenyl)-3-methyl-3H-pyrazolo[3,4-c]quinolin-8-yl)pyridine-2-yl)acetamide (1293)

To a solution of 2-(4-(8-(6-aminopyridin-3-yl)-3-methyl-3H-pyrazolo[3,4-c]quinolin-1-yl)phenyl)-2-methylpropanenitrile (20 mg, 0.05 mmol) in DCM (5 mL) was added Py (0.5 mL) and Ac₂O (10 mg, 0.1 mmol). The reaction mixture was stirred for 15 h at rt and evaporated to dryness. The residue was purified by chromatography (MeOH:DCM1:20) to give compound target product (12 mg, 56%) as brown solid. MS (m/z) (M⁺+H): 461. ¹H-NMR: (δ, ppm, MeOH-D4, 400 Hz) 10.07 (s, 1H), 9.16 (d, 1H, J=2.15 Hz), 8.99 (d, 1H, J=1.76 Hz), 8.84 (d, 1H, J=1.57 Hz), 8.46 (d, 1H, J=8.60 Hz), 8.22 (d, 1H, J=8.71 Hz), 8.16 (d, 1H, J=1.76 Hz), 8.01 (d, 2H, J=8.41 Hz), 7.91 (d, 2H, J=8.44 Hz), 4.34 (s, 3H), 2.30 (s, 3H), 1.84 (s, 6H).

### Example 22

### N-(5-(1-(4-(2-cyanopropan-2-yl)phenyl)-3-methyl-3H-pyrazolo[3,4-c]quinolin-8-yl)pyridine-2-yl) methanesulfonamide (1294)

To a solution of 2-(4-(8-(6-aminopyridin-3-yl)-3-methyl-3H-pyrazolo[3,4-c]quinolin-1-yl)phenyl)-2-methylpropanenitrile (20 mg, 0.05 mmol) in DCM (5 mL) was added Py (0.5 mL) and MsCl (6 mg, 0.05 mmol). The reaction mixture was stirred for 2 h at rt and evaporated to dryness. The residue was purified by chromatography ((MeOH:DCM=1:20)) to give compound target product (13 mg, 61%) as brown solid. MS (m/z) (M⁺+H): 497. ¹H-NMR: (δ, ppm, MeOH-D4, 400 Hz) 9.26 (s, 1H), 8.66 (s, 2H), 8.44 (d, 1H, J=14.65 Hz), 8.20 (d, 1H, J=8.61 Hz), 8.04-7.94 (m, 1H), 7.94-7.91 (m; 2H), 7.77 (d, 1H, J=8.22 Hz), 7.60-7.58 (m, 2H), 4.14 (s, 3H), 3.06 (s, 3H), 1.87 (s, 6H).

### Example 23

### 2-methyl-2-(4-(3-methyl-8-(5-morpholinopyridin-3-yl)-3H-pyrazolo[3,4-c]quinolin-1-yl)phenyl)propanenitrile (1295)

13 mg, 27%. MS (m/z) (M⁺+H): 489. H¹ NMR (δ, ppm, MeOH-d⁴, 400 MHz): 9.98 (s, 1H), 8.43-8.38 (m, 2H), 8.29 (s, 1H), 8.19-8.17 (m, 1H), 8.10 (d, 2H, J=12.40 Hz), 7.93 (d, 2H, J=8.40 Hz), 7.88 (d, 2H, J=8.80 Hz), 4.28 (s, 3H), 3.91-3.89 (m, 4H), 3.49-3.43 (m, 4H), 1.85 (s, 6H).

### Example 24

### 2-methyl-2-(4-(3-methyl-8-(5-(4-methylpiperazin-1-yl)pyridin-3-yl)-3H-pyrazolo[3,4-c]quinolin-1-yl)phenyl)propanenitrile (1296)

15 mg, 30%). MS (m/z) (M⁺+H): 502. ¹H-NMR (δ, ppm, MeOH-d₄, 400MHz): 10.04 (s, 1H), 8.56 (d, 1H, J=2.57 Hz), 8.43-8.23 (m, 4H), 8.10 (d, 1H, J=1.47 Hz), 7.96-7.90 (m, 4H), 4.33-4.28 (m, 5H), 3.78-3.72 (m, 2H), 3.49-3.43 (m, 2H), 3.38-3.35 (m, 2H), 3.01 (s, 3H), 1.80 (s, 6H).

### Example 25

### 5-(1-(4-(2-cyanopropan-2-yl)phenyl)-3-methyl-3H-pyrazolo[3,4-c]quinolin-8-yl)-N-cyclopropylpicolinamide (1297)

42 mg, yeild: 70%. MS (m/z) (M⁺+H): 487. ¹H-NMR (δ, ppm, CDCl3, 400MHz): 9.23 (s, 1H), 8.70 (s, 1H), 8.41 (s, 1H), 8.34 (d, J=8.4 Hz, 1H), 8.23 (d, J=8.0 Hz, 1H), 8.06 (br. s, 1H), 7.85-7.99 (m, 4H), 7.72 (s, 1H), 7.70 (s, 1H), 4.37 (s, 3H), 2.96-2.98 (m, 1H), 1.84 (s, 6H), 0.89-0.91 (m, 2H), 0.69-0.71 (m, 2H).

### Example 26

### 5-(1-(4-(2-cyanopropan-2-yl)phenyl)-3-methyl-3H-pyrazolo[3,4-c]quinolin-8-yl)-N-cyclopropylnicotinamide (1298)

15 mg, 30%). MS (m/z) (M⁺+H): 487. H¹ NMR (δ, ppm, CDCl₃, 400 MHz): 9.33 (s, 1H), 8.77 (bs, 2H), 8.21 (d, 1H, J=4.40 Hz), 8.19 (s, 1H), 7.84-7.77 (m, 4H), 7.66 (d, 1H, J=8.00 Hz), 6.49 (s, 1H), 4.14 (s, 3H), 2.95-2.93 (m, 1H), 1.87 (s, 6H), 0.94-0.89 (m, 2H), 0.69-0.65 (m, 1H).

### Example 27

### 2-(4-(8-(5-(dimethylamino)□yridine-3-yl)-3-methyl-3H-pyrazolo[3,4-c]quinolin-1-yl)phenyl)-2-methylpropanenitrile (1299)

17 mg, 38%). MS (m/z) (M⁺+H): 447. H¹ NMR (δ, ppm, CDCl₃, 400MHz): 9.32 (s, 1H), 8.20 (d, 1H, J=8.40 Hz), 8.08 (d, 1H, J=2.80 Hz), 8.02 (d, 1H, J=3.20 Hz), 7.81-7.75 (m, 3H), 7.66-7.64 (m, 2H), 7.00 (s, 1H), 4.10 (s, 3H), 3.00 (s, 6H), 1.84 (s, 6H).

### Example 28

### 5-(1-(4-(2-cyanopropan-2-yl)phenyl)-3-methyl-3H-pyrazolo[3,4-c]quinolin-8-yl)-N-methyl nicotinamide (1300)

17 mg, 37%). MS (m/z) (M⁺+H): 461. H¹ NMR (δ, ppm, CDCl₃, 400 MHz): 9.58 (s, 1H), 8.96 (d, 1H, J=7.60 Hz), 8.58 (s, 1H), 8.23 (d, 1H, J=8.00 Hz), 8.00 (d, 2H, J=8.40 Hz), 7.90-7.84 (m, 4H), 7.70 (d, 2H, J=7.60 Hz), 4.30 (s, 3H), 3.08 (s, 1H), 1.86 (s, 6H).

### Example 29

### 2-(4-(8-(5-(isopropylamino)□yridine-3-yl)-3-methyl-3H-pyrazolo[3,4-c]quinolin-1-yl)phenyl)-2-methylpropanenitrile (1301)

Yield: 26%. MS (m/z) (M⁺+H): 461. H¹ NMR (δ, ppm, CDCl3, 400 MHz): 9.21 (s, 1H), 8.39 (d, 1H), 8.31 (d, 1H), 8.13 (s, 1H), 7.98 (1H), 7.89 (d, 1H), 7.87 (dd, 2H), 7.69 (d, 2H), 6.98 (s, 1H), 4.36 (s, 3H), 1.82 (s, 6H), 1.27 (d, 6H).

### Example 30

### 2-(4-(8-(5-hydroxypyridin-3-yl)-3-methyl-3H-pyrazolo[3,4-c]quinolin-1-yl)phenyl)-2-methylpropanenitrile (1302)

Yield: 12%. MS (m/z) (M⁺+H): 420. ¹H-NMR (δ, ppm, CDCl3, 400 MHz): 9.21 (s, 1H), 8.47 (d, 1H), 8.29 (m, 3H), 7.92 (dd, 1H), 7.87 (d, 2H), 7.74 (d, 2H), 7.25 (t, 1H), 4.37 (s, 3H), 1.86 (s, 6H).

### Example 31

### N-(5-(1-(4-(2-cyanopropan-2-yl)phenyl)-3-methyl-3H-pyrazolo[3,4-c]quinolin-8-yl)pyridine-3-yl)cyclopropanecarboxamide (1303)

16 mg, 33%. MS (m/z) (M⁺+H): 487. H¹ NMR (δ, ppm, CDCl₃, 400 MHz): 11.50 (bs, 1H), 9.88 (bs, 1H), 9.01 (bs, 1H), 8.61 (bs, 1H), 8.53-8.41 (m, 2H), 8.08 (bs, 1H), 7.93-7.87 (m, 5H), 4.22 (s, 3H), 1.99-1.97 (m, 1H), 1.78 (s, 6H), 0.92-0.90 (m, 4H).

### Example 32

### 5-(1-(4-(2-cyanopropan-2-yl)phenyl)-3-methyl-3H-pyrazolo[3,4-c]quinolin-8-yl)-N-methyl picolinamide (1304)

31 mg, □yrid: 56%. MS (m/z) (M⁺+H): 461. ¹H-NMR (δ, ppm, CDCl₃, 400 MHz): 9.22 (s, 1H), 8.71 (s, 1H), 8.41 (s, 1H), 8.33 (d, 1H), 8.22 (d, 1H), 8.03 (br. S, 1H), 7.85-7.99 (m, 4H), 7.72 (s, 1H), 7.70 (s, 1H), 4.37 (s, 3H), 3.06 (d, 3H), 1.84 (s, 6H).

IV. Preparation of Intermediates and Final Compounds of Scaffold R

Pyrazolo[3,4-c]quinoline derivatives with variation on 1-position is classified as Scaffold R. There are two synthesis Routes (Route 1 and Route 2) to prepare key intermediate, 1-bromo-8-(pyridin-3-yl)-3H-pyrazolo[3,4-c]quinoline (12), which is used for preparing 1-substituted pyrazolo[3,4-c]quinoline derivatives.

IV-1. Preparation of Intermediates for Scaffold R

Route 1. Preparation of Intermediates for Scaffold R

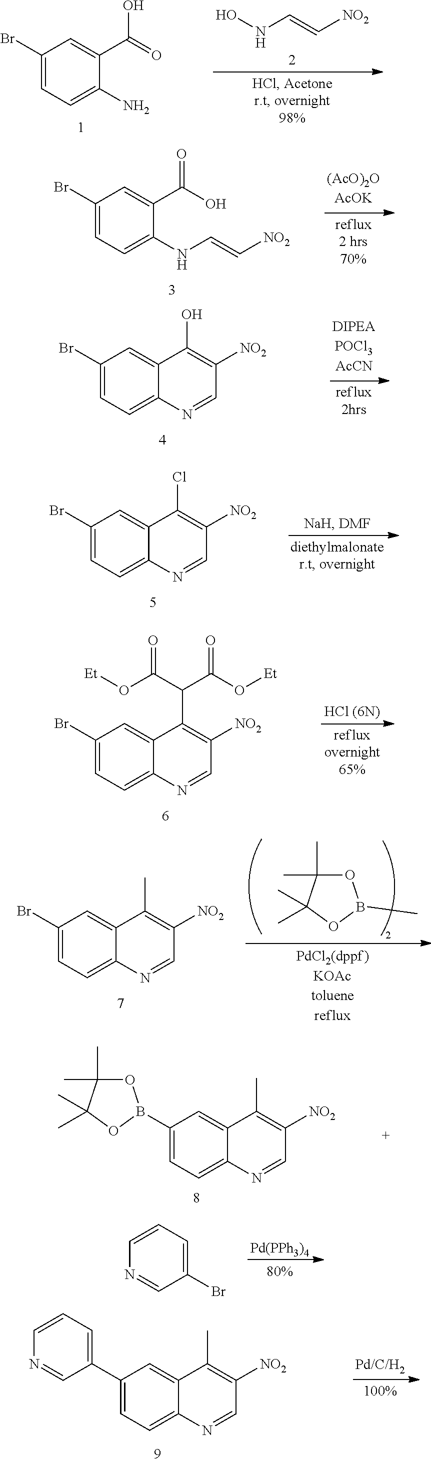

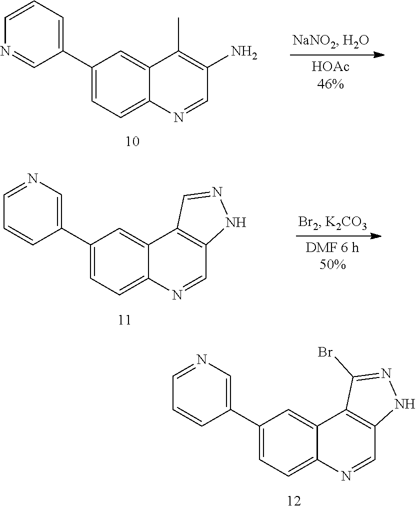

### (E)-N-hydroxy-2-nitroethenamine (2)

A solution of sodium hydroxide (112 g, 2.8 mol) in water (250 mL) was cooled and stirred at room temperature, to which, nitromethane (61 g, 1.0 mol) was added dropwisely at room temperature and slowly raised to 45° C. for 5 min then cooled to room temperature. Another half amount of nitromethane (61 g, 1.0 mol) was added dropwisely at 45° C. The mixture was stirred for 10 min till clear red solution was obtained. The solution was then heated to 50° C. for 5 min and finally cooled to room temperature, poured onto crashed ice (600 g), and acidified with concentrated hydrogen chloride. The resultant solution of methazonic acid 2 was immediately used for next step.

### (E)-5-Bromo-2-(2-nitrovinylamino)benzoic acid (3)

Compound 2 was immediately added to a filtered solution of 5-bromoanthranilic acid (23.76 g, 0.11 mol) and 500 ml of conc. HCl in 1000 ml water. The solution was allowed to stand at room temperature for 18 hours, and then filtered. The solid product was washed repeatedly with water. The cake was sliced into thin flakes and allowed to dry at room temperature to give compound 3 (26 g, 91%). MS (m/z) (M⁺+H): 287, 289.

### 6-Bromo-3-nitroquinolin-4-ol (4)

Compound 3 (15 g, 0.052 mol) and potassium acetate (6.16 g, 0.063 mol) in acetic anhydride (100 mL) were stirred for 1.5 h at 120° C. The precipitate was filtered and washed with acetic acid until the filtrate was colorless and then with water. The solid was dried to give 4 (6 g, 43%). MS (m/z) (M⁺+H): 269, 271.

### 6-Bromo-4-chloro-3-nitroquinoline (5)

To a solution of 4 (15 g, 0.056 mol) in acetonitrile (80 mL) and DIPEA (15.9 g, 0.123 mol), was added POCl₃ (17.1 g, 0.112 mol) dropwisely at 0° C. The reaction temperature was slowly raised to 100° C. for 2 hours. The mixture was cooled and poured onto ice-water. After Neutralized with aq NaHCO₃, extracted with ethyl acetate, and dried over Na₂SO₄, the crude product 5 was obtained by evaporating of solution to dryness (15 g, 93%) as a brown solid. MS (m/z) (M⁺+H): 287, 289.

### Diethyl 2-(6-bromo-3-nitroquinolin-4-yl)malonate (6)

To a mixture of NaH (3.5 g, 140 mmol) in DMF (100 ml) was added Diethyl malonate (11 ml, 70 mmol) dropwise at r.t. The reaction mixture was heated to 80° C. for 1 h. 6-bromo-4-chloro-3-nitroquinoline (20 g, 0.07 mol) was added to the above mixture. The mixture was stirred at rt overnight. The mixture was quenched with water (300 ml) and extracted with EA (3×100 ml). The organic layers were combined and washed with water (3×100 ml), dried over MgSO₄, and filtered. The filtration was concentrated to give 6 (20 g, 70%) as a light yellow solid.

### 6-Bromo-4-methyl-3-nitroquinoline (7)

A solution of the compound 6 (30 g, 76 mol) and HCl (6N, 100 ml) was heated to reflux overnight. The mixture was neutralized with sodium hydroxide (30%) carefully, and extracted with EA (3×100 mL). The organic layers were combined and washed with brine, dried over Na₂SO₄, filtered. The filtrate was concentrated to give 7 (12 g, 43%) as a light yellow solid. MS (m/z) (M⁺+H): 267, 269. ¹H-NMR: (δ, ppm, CDCl₃, 400 Hz): 9.23 (s, 1H), 8.35 (m, 1H), 8.01 (m, 1H), 7.94 (m, 1H), 2.89 (s, 1H)

### 4-Methyl-3-nitro-6-(4,4,5,5-tetramethyl-1,3,2-dioxaborolan-2-yl)quinoline (8) and 4-methyl-3-nitro-6-(pyridin-3-yl)quinoline (9)

To a solution of 7 (7 g, 26.3 mmol) in toluene (100 ml), was added PdCl₂(dppf) (0.05 eq.), bis(pinacolate)diboron (10 g, 39.5 mmol), KOAc (7.7 g, 78.9 mmol) under N₂. The solution was heated to reflux overnight and cooled. To the above solution of (4-methyl-3-nitro-6-(4,4,5,5-tetramethyl-1,3,2-dioxaborolan-2-yl)quinoline) was added K₂CO₃ (10.9 g, 78.9 mmol), 3-bromopyridine (6.2 g, 39.5 mmol) and PdCl₂(dppf) (cat). The mixture was heated to 85° C. for 6 h and cooled. The solution was extracted with EA (2×30 ml), washed with brine (2×20 ml), dried over Na₂SO₄ and filtered. The filtrate was concentrated in vacuo, and re-crystallized (EA/ether) to give compound 9 (4 g,: 57%). MS (m/z) (M⁺+H): 266.

### 4-Methyl-6-(pyridin-3-yl)quinolin-3-amine (10)

The mixture of 9 (4 g, 15.1 mmol) and Pd/C (1.5 g) in THF (100 ML) was stirred under H₂ at rt for 2 h. The mixture was filtrated. The filtrate was concentrated in vacuo to give compound 10 (3.5 g,: 100%). MS (m/z) (M⁺+H): 236.

### 8-(Pyridin-3-yl)-3H-pyrazolo[3,4-c]quinoline (11, Example 1)

To a solution of 10 (3.5 g, 14.9 mmol) in acetic acid (200 ml) was added a solution of NaNO₂ (1.2 g, 17.9 mmol) in 5 mL of water dropwise at room temperature. The mixture was stirred at room temperature overnight. The solvent was removed by reduced pressure. The residue was purified by column chromatography to give 8-(pyridin-3-yl)-3H-pyrazolo[3,4-c]quinoline (11) (1.7 g, 46%). The best yield for this reaction was 90% when started from 350 mg compound 10 MS (m/z) (M⁺+H): 247. ¹H-NMR (δ, DMSO-d6, 400 MHz, ppm): 14.05 (s, 1H), 9.31 (s, 1H), 9.17 (s, 1H), 8.84 (m, 2H), 8.67 (d, 2H, J=3.9 Hz), 8.37 (d, 1H, J=6.82 Hz), 8.23 (d, 1H, J=8.78 Hz), 8.06 (d, 1H, J=7.31 Hz), 7.61 (m, 1H).

### 1-Bromo-8-(pyridin-3-yl)-3H-pyrazolo[3,4-c]quinoline (12)

To a mixture of 11 (100 mg, 0.41 mmol) and K₂CO₃ (168 mg, 1.22 mmol) in DCM (5 ml) was added Br₂ (130 mg, 0.81 mmol) dropwise at room temperature. The mixture was stirred at room temperature for 6 h. 5 ml of H₂O was added. The organic layer was separated and washed with brine (2×5 ml), dried over MgSO₄, and filtered. The filtrate was concentrated and purified by flash chromatography (silica column, using EA:PE1:1) to afford 12 (70 mg, 50%). MS (m/z) (M⁺+H): 325, 327. ¹H-NMR (δ, DMSO-d6, 400 MHz, ppm): 9.09 (s, 1H), 8.99 (s, 1H), 8.92 (s, 1H), 8.61 (s, 1H), 8.19-8.17 (d, 1H, J=8.2 Hz), 8.10-8.07 (m, 1H), 7.83-7.80 (m, 1H), 7.57-7.53 (m, 1H).

Route 2. Preparation of Intermediates for Scaffold R

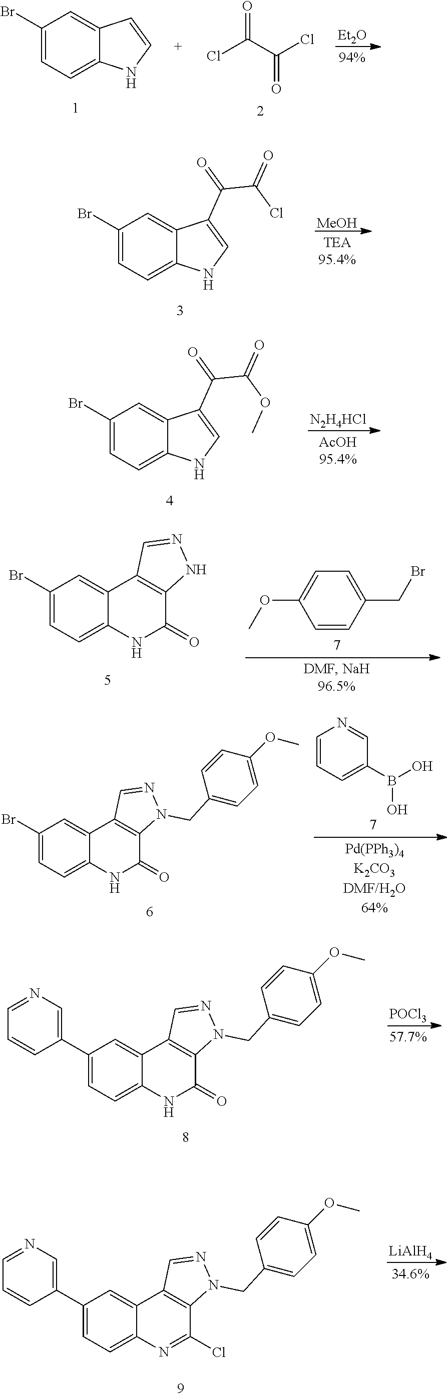

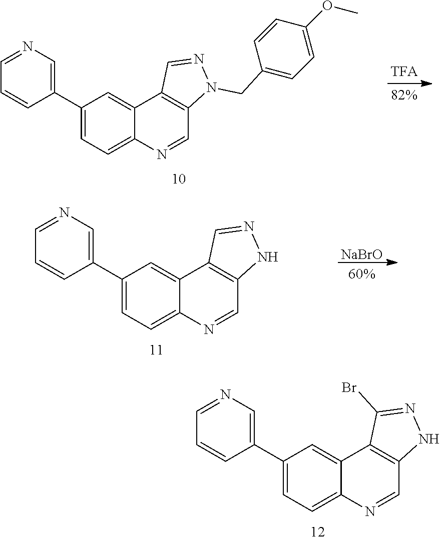

### 2-(5-Bromo-1H-indol-3-yl)-2-oxoacetyl chloride (3)

To a solution of 1 (40 g, 0.20 mol) in Et₂O (200 mL) was added 2 (100 mL) in Et₂O (200 mL) slowly at 0° C. The reaction mixture was stirred at 0□ for 1 hour. The solid was filtered and washed with Et₂O (100 mL) to afford 3 (55 g, 94%) as a yellow solid. MS (m/z) (M⁺+H): 286).

### Methyl 2-(5-bromo-1H-indol-3-yl)-2-oxoacetate (4)

To a solution of 3 (55 g, 0.192 mol) in methanol (300 mL) was added Et₃N (58 g, 0.58 mol) slowly at 0□. The reaction mixture was stirred at room temperature overnight. The solid was filtered and washed with methanol (100 mL×2) to afford 4 (51.5 g, 95%) as a yellow solid. MS (m/z) (M⁺+H): 282.

### 8-Bromo-3H-pyrazolo[3,4-c]quinolin-4(5H)-one (5)

To a solution of 4 (16.5 g, 58 mmol) in AcOH (100 mL) was added the hydrazine hydrochloride (40 g, 58 mmol). The reaction mixture was stirred at 120□ for 2 days. The reaction mixture was cooled and the solid was filtered. The collected solid was then washed with water (100 mL) and ethanol (100 mL×2) to afford 5 (51.5 g, 95%) as a gray solid. MS (m/z) (M⁺+H): 264.

### 8-Bromo-3-(4-methoxybenzyl)-3H-pyrazolo[3,4-c]quinolin-4(5H)-one (6)

To a solution of 5 (10 g, 37.8 mmol) in DMF (150 mL) was added NaH (1.8 g, 45.4 mmol, 60% in mineral oil) slowly at 0° C. The mixture was stirred for 30 min and then was added 1-(bromomerhyl)-4-methoxybenzene (9.1 g, 45.4 mmol) slowly. The resulted reaction mixture was stirred at room temperature overnight before it was concentrated in vacuo. The residue was poured into water (500 mL) and the crashed solid was filtered, which was washed with water and dried to afford 6 (14 g, 97%) as a pale solid. MS (m/z) (M⁺+H): 384.

### 3-(4-Methoxybenzyl)-8-(pyridin-3-yl)-3H-pyrazolo[3,4-c]quinolin-4(5H)-one (8)

To a solution of 6 (5.0 g, 13.0 mmol) and pyridine-3-ylboronic acid (1.9 g, 15.6 mmol) in DMF/H₂O (50 ml/5 ml) was added Pd(PPh₃)₄ (1.05 g, 0.91 mmol) and K₂CO₃ (5.4 g, 39 mmol). The reaction mixture was stirred at 100° C. under N₂ overnight before it was concentrated in vacuo. The resulted residue was poured into water (500 mL) and the crashed solid was filtered and washed with EtOAc (50 mL×2) to afford 8 (3.2 g, 64%) as a pale solid. MS (m/z) (M⁺+H): 383.

### 4-Chloro-3-(4-methoxybenzyl)-8-(pyridin-3-yl)-3H-pyrazolo[3,4-c]quinoline (9)

To a solution of 8 (1 g, 2.6 mmol) was added POCl₃ (10 mL) and the reaction mixture was refluxed for 2 hours before it was concentrated in vacuo. The resulted residue was washed with THF (10 mL) and CH₂Cl₂ (10 mL) to afford a yellow solid. The yellow solid was then mixed with 5% NaHCO₃ (sat.) and the mixture was extracted with CH₂Cl₂ (50 mL×4). The combined organic layers were dried over Na₂SO₄ and concentrated in vacuo to afford 9 (0.60 g, 58%) as a yellow solid. MS (m/z) (M⁺+H): 401.

### 3-(4-Methoxybenzyl)-8-(pyridin-3-yl)-3H-pyrazolo[3,4-c]quinoline (10)

To a solution of 9 (0.6 g, 1.5 mmol) in THF (20 mL) was added LiAlH₄ (0.57 g, 15 mmol), and the reaction mixture was stirred at room temperature for 2 hours. The reaction mixture was diluted with diethyl ether and quenched by Na₂SO₄.10H₂O. The reaction solution was poured out and concentrated in vacuo to afford the crude product. To a solution of the crude product in AcOH (20 mL) was added DDQ. The reaction mixture was stirred for 10 minutes before it was concentrated in vacuo. The resulted residue was added to 10% Na₂CO₃ (aq.) and the mixture was extracted with EtOAc (50 mL×2). The combined organic layers were concentrated in vacuo to afford the crude product which was purified by column chromatography (petroleum ether/ethyl acetate 1/4, 0.5% triethyl amine) to afford 10 (0.19 g, 34.6%) as a yellow solid. MS (m/z) (M⁺+H): 367.

### 8-(Pyridin-3-yl)-3H-pyrazolo[3,4-c]quinoline (11)

To a solution of 10 (0.19 g, 0.52 mmol) was added TFA (5 mL) and the reaction mixture was stirred at 50□ for 2 hours before it was concentrated in vacuo. The residue was then added 10% Na₂CO₃ (aq.) and the mixture was extracted with EtOAc (50 mL×2). The combined organic layers were dried over Na₂SO₄ and concentrated in vacuo to afford 11 (0.105 g, 82%) as a brown solid. MS (m/z) (M⁺+H): 247.

### 1-Bromo-8-(pyridin-3-yl)-3H-pyrazolo[3,4-c]quinoline (12)

To a solution of 1M NaOH (4.2 mL) was added bromine (67 mg) and the mixture was stirred for ten minutes, followed by an addition of 11 (0.105 g, 0.42 mmol) in CH₂Cl₂ (0.5 mL). The reaction mixture was stirred at room temperature for 30 minutes before it was diluted with H₂O (5 mL). 1M HCl was then added to adJ=ust the pH to 7˜8. The reaction mixture was extracted with CH₂Cl₂/MeOH(10/1, 5 mL×2). The combined organic layers were dried over Na₂SO₄ and concentrated in vacuo to afford 12 (0.082 g, 60%) as a yellow solid.

Other key intermediates (25 to 28, 13, and 14) in the following Scheme were prepared by N-alkylation on N2- or N3-position of pyrazolo[3,4-c]quinoline core and the procedures were similar to preparation of the intermediates in Scaffold J=. The chemical structures of the intermediates were also confirmed by NOE.

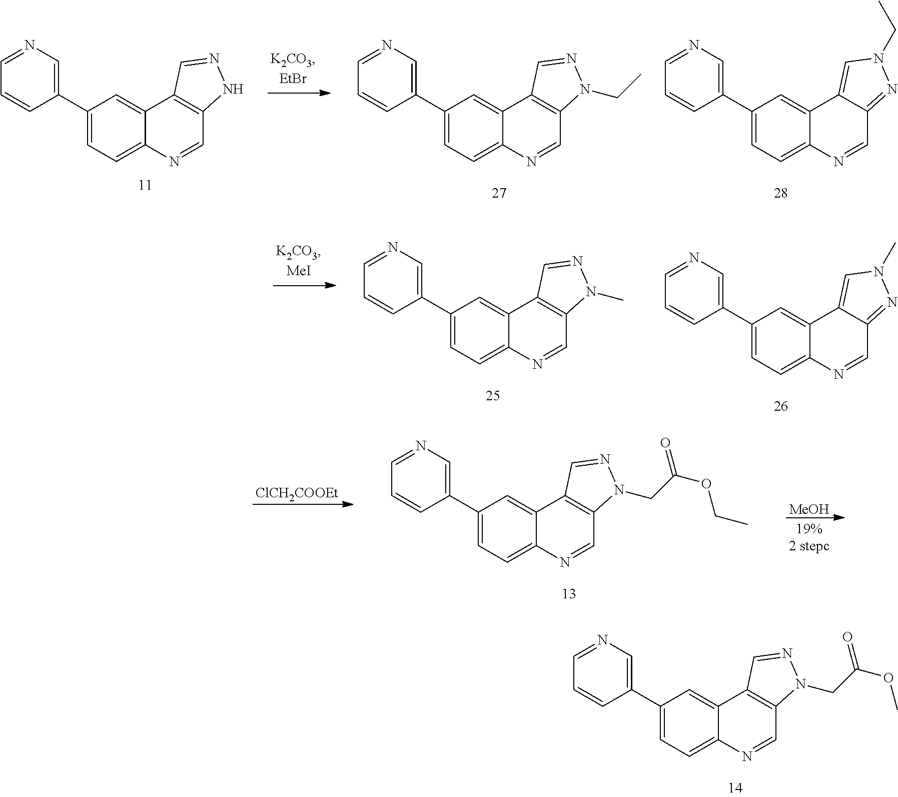

IV-2. Preparation of Target Compounds of Scaffold R

The pyrazolo[3,4-c]quinoline derivatives with variation on 1-position were prepared in multiple methods as shown in the following Schemes. All final compounds in Scaffold R were summarized in Table IV.

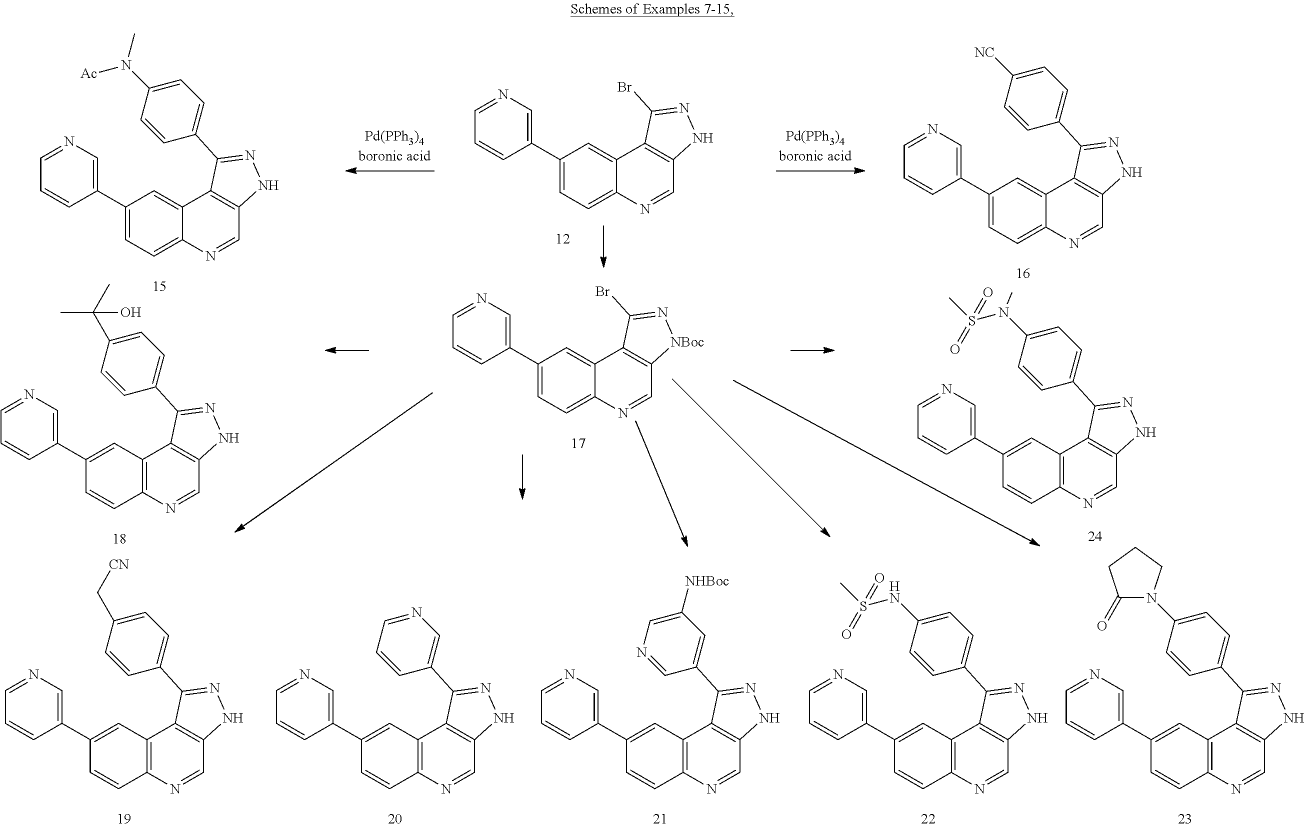

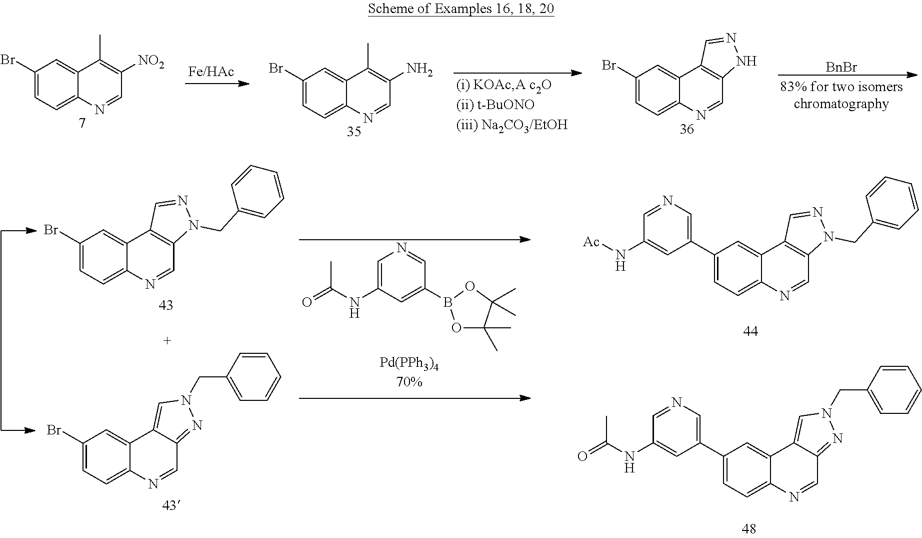

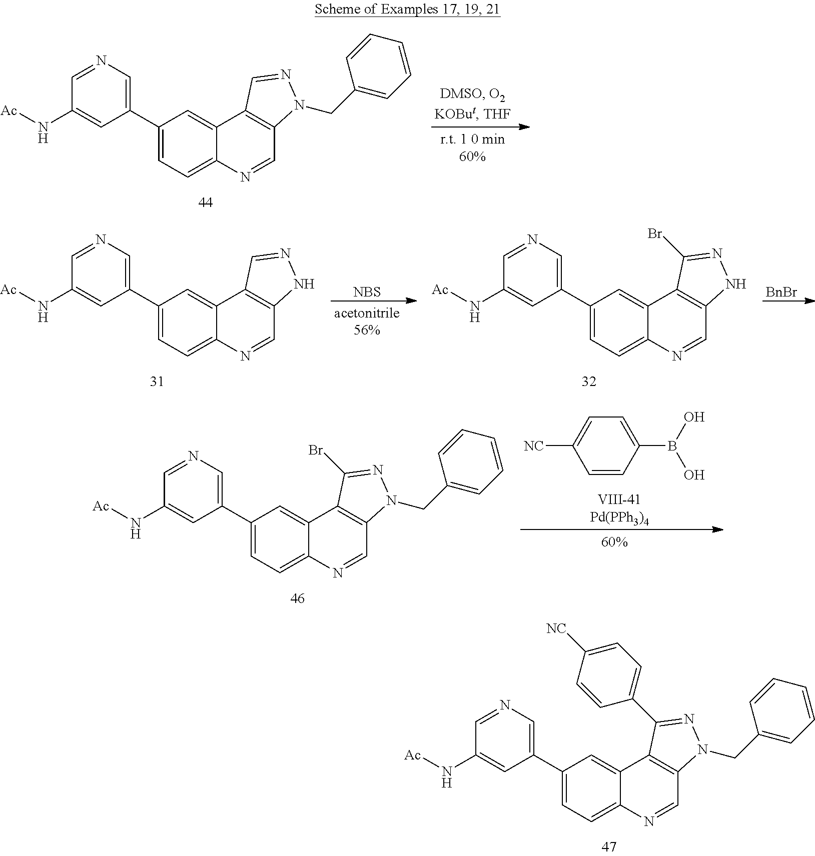

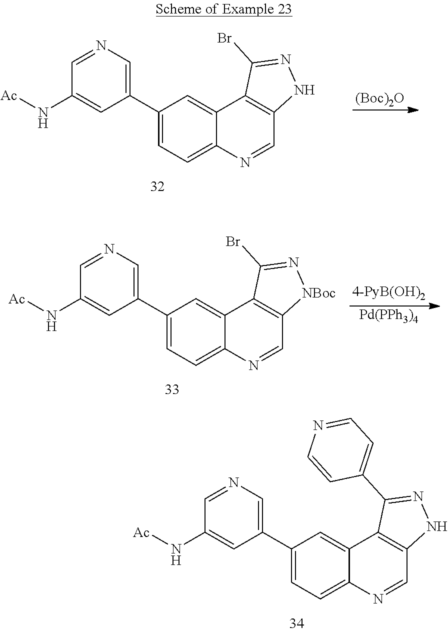

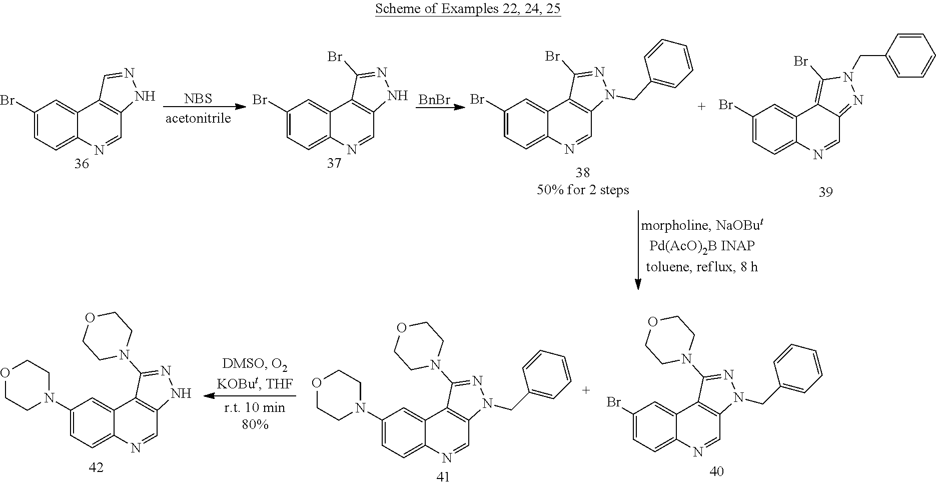

| | Compound # in | | | |
|---|---|---|---|---|
| Entry | Schemes | Structure | Method | Yield (%) |
| 1 | 11 | 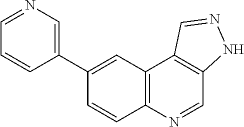 | NaNO₂/HCl | 46% |
| 2 | 14 | 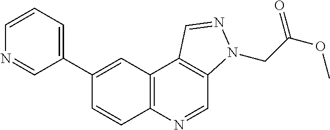 | BrCH₂COOEt/ MeOH | 19% |
| 3 | 25 | 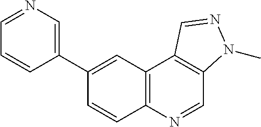 | MeI/EtOH, RT | 14% |
| 4 | 26 | 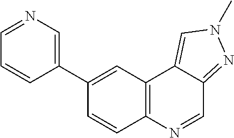 | MeI/EtOH, RT | 13% |
| 5 | 27 | 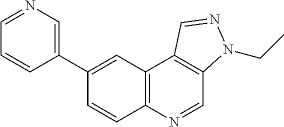 | EtBr/EtOH, 50° C. | 9% |
| 6 | 28 | 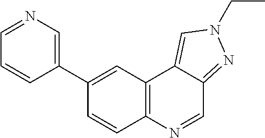 | EtBr/EtOH, 50° C. | 7% |
| 7 | 15 | 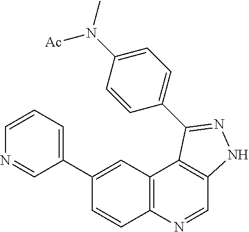 | Pd(PPh₃)₄, DMF/H₂O, 120° C., Na₂CO₃ | 7% |
| 8 | 16 | 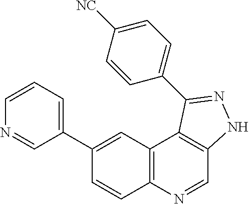 | Pd(PPh₃)₄, DMF/H₂O, 120° C., Na₂CO₃ | 13% |
| 9 | 18 | 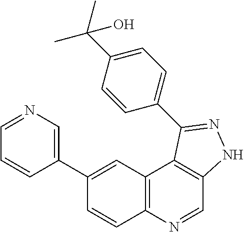 | Pd(PPh₃)₄, DMF/H₂O, 120° C., Na₂CO₃ | 17% |
| 10 | 19 | 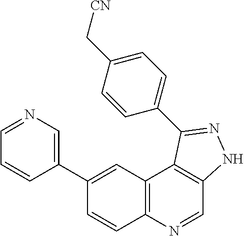 | Pd(PPh₃)₄, DMF/H₂O, 120° C., Na₂CO₃ | 30% |
| 11 | 20 | 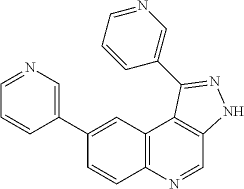 | Pd(PPh₃)₄, DMF/H₂O, 120° C., Na₂CO₃ | 40% |
| 12 | 21 | 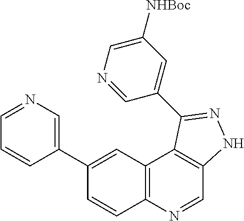 | Pd(PPh₃)₄, DMF/H₂O, 120° C., Na₂CO₃ | 15% |
| 13 | 22 | 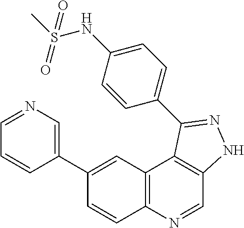 | Pd(PPh₃)₄, DMF/H₂O, 120° C., Na₂CO₃ | 9% |
| 14 | 23 | 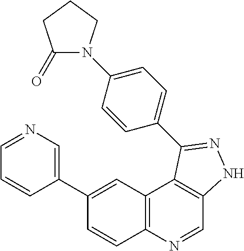 | Pd(PPh₃)₄, DMF/H₂O, 120° C., Na₂CO₃ | 13% |
| 15 | 24 | 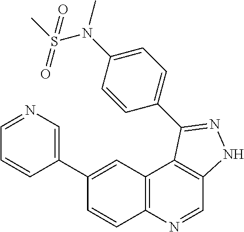 | Pd(PPh₃)₄, DMF/H₂O, 120° C., Na₂CO₃ | 25% |
| 16 | 36 | 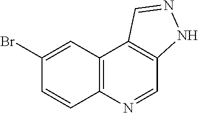 | NaNO₂/HAc | 10% |
| 17 | 32 | 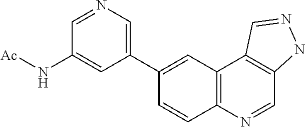 | KOAc, Ac₂O, t-BuONO | 60% |
| 18 | 44 | 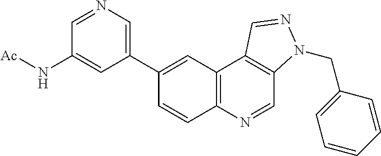 | Pd(dppf)Cl₂, DMF/H₂O, 150° C., K₂CO₃ | 65% |
| 19 | 46 | 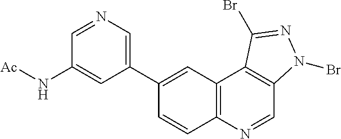 | BnBr | 79% |
| 20 | 48 | 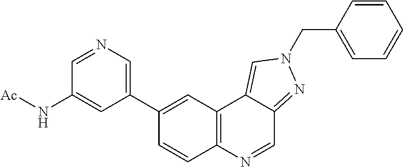 | Pd(dppf)Cl₂, DMF/H₂O, 150° C., K₂CO₃ | 65% |
| 21 | 47 | 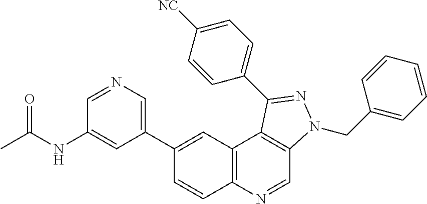 | Pd(dppf)Cl₂, DMF/H₂O, 150° C., K₂CO₃ | 60% |
| 22 | 40 | 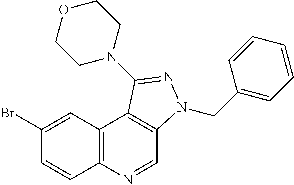 | Pd(AcO)₂ BINAP | 30% |
| 23 | 34 | 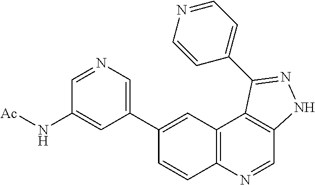 | Pd(dppf)Cl₂, DMF/H₂O, 150° C., K₂CO₃ | 59% |
| 24 | 41 | 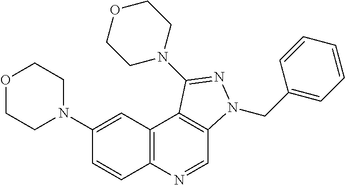 | Pd(AcO)₂, BINAP | 60% |
| 25 | 42 | 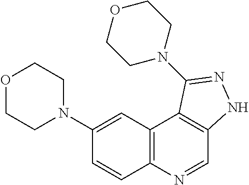 | DMSO, O₂, KOBu^{t} | 70% |

TABLE IV

| Cpd | | | | MS | |
|---|---|---|---|---|---|
| No | Ex | Structure | MF/MW | (M⁺ + H) | IUPAC |
| 1305 | 1 | 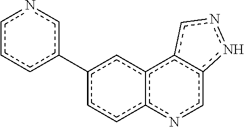 | C15H10N4/ 246.3 | 247 | 8-(pyridin-3-yl)-3H- pyrazolo[3,4- c]quinoline |
| 1306 | 2 | 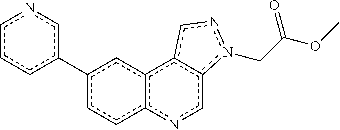 | C18H14N4O2/ 318.3 | 319 | methyl 2-(8-(pyridin-3- yl)-3H-pyrazolo[3,4- c]quinolin-3-yl)acetate |
| 1307 | 3 | 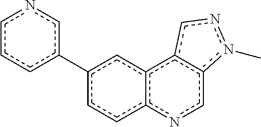 | C16H12N4/ 260.3 | 261 | 3-methyl-8-(pyridin-3- yl)-3H-pyrazolo[3,4- c]quinoline |
| 1308 | 4 | 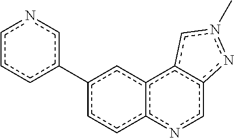 | C16H12N4/ 260.3 | 261 | 2-methyl-8-(pyridin-3- yl)-2H-pyrazolo[3,4- c]quinoline |
| 1309 | 5 | 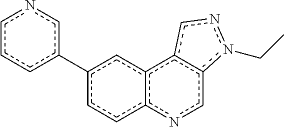 | C17H14N4/ 274.3 | 275 | 3-ethyl-8-(pyridin-3- yl)-3H-pyrazolo[3,4- c]quinoline |
| 1310 | 6 | 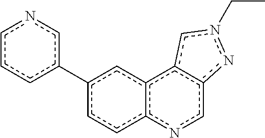 | C17H14N4/ 274.3 | 275 | 2-ethyl-8-(pyridin-3- yl)-2H-pyrazolo[3,4- c]quinoline |
| 1311 | 7 | 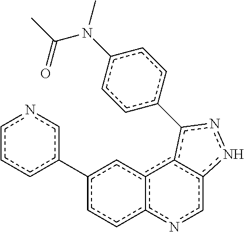 | C24H19N5O/ 393.4 | 394 | N-methyl-N-(4-(8- (pyridin-3-yl)-3H- pyrazolo[3,4- c]quinolin-1- yl)phenyl)acetamide |
| 1312 | 4 | 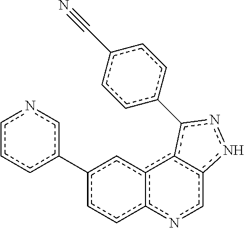 | C22H13N5/ 347.4 | 348 | 4-(8-(pyridin-3-yl)-3H- pyrazolo[3,4- c]quinolin-1- yl)benzonitrile |
| 1313 | 9 | 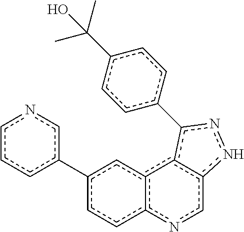 | C24H20N4O/ 380.4 | 381 | 2-(4-(8-(pyridin-3-yl)- 3H-pyrazolo[3,4- c]quinolin-1- yl)phenyl)propan-2-ol |
| 1314 | 10 | 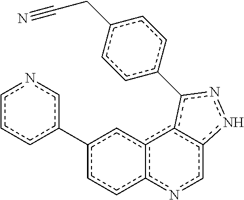 | C23H15N5/ 361.4 | 362 | 2-(4-(8-(pyridin-3-yl)- 3H-pyrazolo[3,4- c]quinolin-1- yl)phenyl)acetonitrile |
| 1315 | 11 | 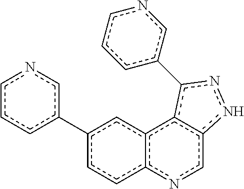 | C20H13N5/ 323.4 | M + Na 346 | l,8-di(pyridin-3-yl)- 3H-pyrazolo[3,4- c]quinoline |
| 1316 | 12 | 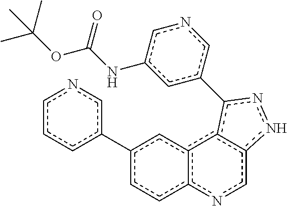 | C25H22N6O2/ 438.5 | 439 | tert-butyl 5-(8-(pyridin- 3-yl)-3H-pyrazolo[3,4- c]quinolin-1-yl)pyridin- 3-ylcarbamate |
| 1317 | 13 | 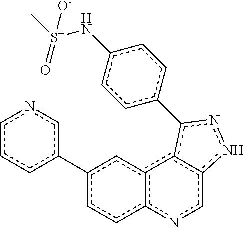 | C22H17N5O2S/ 415.5 | 416 | N-(4-(8-(pyridin-3-yl)- 3H-pyrazolo[3,4- c]quinolin-1- yl)phenyl) methanesulfonamide |
| 1318 | 14 | 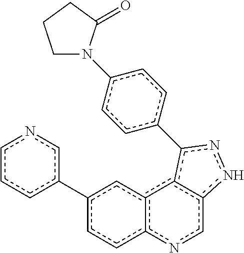 | C25H19N5O/ 405.5 | 406 | 1-(4-(8-(pyridin-3-yl)- 3H-pyrazolo[3,4- c]quinolin-1- yl)phenyl)pyrrolidin-2- one |
| 1319 | 15 | 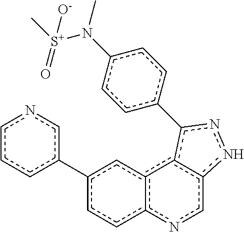 | C23H19N5O2S/ 429.5 | 430 | N-methyl-N-(4-(8- (pyridin-3-yl)-3H- pyrazolo[3,4- c]quinolin-1- yl)phenyl) methanesulfonamide |
| 1320 | 16 | 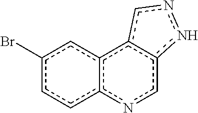 | C10H6BrN3/ 248.1 | 248, 250 | 8-bromo-3H- pyrazolo[3,4- c]quinoline |
| 1321 | 17 | 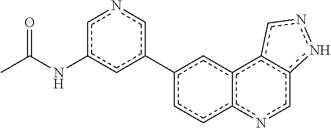 | C17H13N5O/ 303.3 | 304 | N-(5-(3H-pyrazolo[3,4- c]quinolin-8-yl)pyridin- 3-yl)acetamide |
| 1322 | 18 | 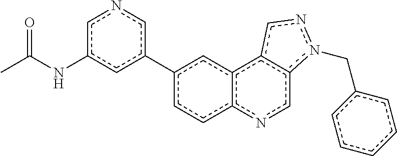 | C24H19N5O/ 393.4 | 394 | N-(5-(3-benzyl-3H- pyrazolo[3,4- c]quinolin-8-yl)pyridin- 3-yl)acetamide |
| 1323 | 19 | 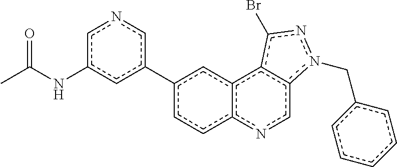 | C24H18BrN5O/ 472.3 | 372, 374 | N-(5-(3-benzyl-1- bromo-3H- pyrazolo[3,4- c]quinolin-8-yl)pyridin- 3-yl)acetamide |
| 1324 | 20 | 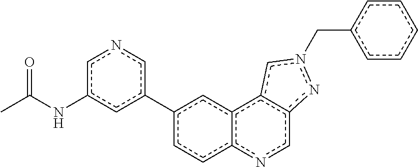 | C24H19N5O/ 393.4 | 394 | N-(5-(2-benzyl-2H- pyrazolo[3,4- c]quinolin-8-yl)pyridin- 3-yl)acetamide |
| 1325 | 21 | 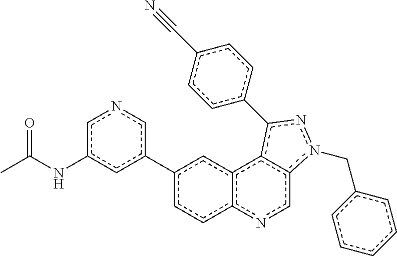 | C31H22N6O/ 494.5 | 495 | N-(5-(3-benzyl-1-(4- cyanophenyl)-3H- pyrazolo[3,4- c]quinolin-8-yl)pyridin- 3-yl)acetamide |
| 1326 | 22 | 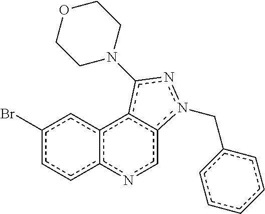 | C21H19BrN4O/ 423.3 | 423, 425 | 3-benzyl-8-bromo-1- morpholino-3H- pyrazolo[3,4- c]quinoline |
| 1327 | 23 | 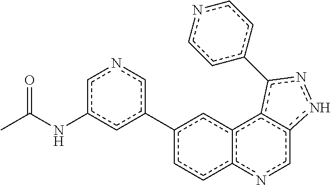 | C22H16N6O/ 380.4 | 381 | N-(5-(1-(pyridin-4-yl)- 3H-pyrazolo[3,4- c]quinolin-8-yl)pyridin- 3-yl)acetamide |
| 1328 | 24 | 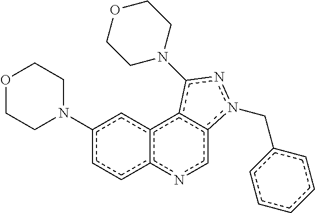 | C25H27N5O2/ 429.5 | 430 | 3-benzyl-1,8- dimorpholino-3H- pyrazolo[3,4- c]quinoline |
| 1329 | 25 | 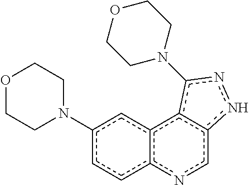 | C18H21N5O2/ 339.4 | 340 | 1,8-dimorpholino-3H- pyrazolo[3,4- c]quinoline |

Synthetic Procedures for Preparing Compound in Table IV.

### Example 1

### 8-(Pyridin-3-yl)-3H-pyrazolo[3,4-c]quinoline (1305)

To a solution of 10 (3.5 g, 14.9 mmol) in acetic acid (200 ml) was added a solution of NaNO₂ (1.2 g, 17.9 mmol) in 5 mL of water dropwise at room temperature. The mixture was stirred at room temperature overnight. The solvent was removed by reduced pressure. The residue was purified by column chromatography to give 8-(pyridin-3-yl)-3H-pyrazolo[3,4-c]quinoline (11) (1.7 g, 46%). The best yield for this reaction was 90% when started from 350 mg compound 10. MS (m/z) (M⁺+H): 247. ¹H-NMR (δ, DMSO-d6, 400 MHz, ppm): 14.05 (s, 1H), 9.31 (s, 1H), 9.17 (s, 1H), 8.84 (m, 2H), 8.67 (d, 2H, J=3.9 Hz), 8.37 (d, 1H, J=6.82 Hz), 8.23 (d, 1H, J=8.78 Hz), 8.06 (d, 1H, J=7.31 Hz), 7.61 (m, 1H).

### Example 2

### ethyl 2-(8-(pyridin-3-yl)-3H-pyrazolo[3,4-c]quinolin-3-yl)acetate (13) and methyl 2-(8-(pyridin-3-yl)-3H-pyrazolo[3,4-c]quinolin-3-yl)acetate (14) (1306)

To a mixture of NaH (52 mg, 60% in oil) in DMF (2 ml) was added a solution of compound 11 (80 mg, 0.33 mmol) in DMF (1 ml) dropwise at 0° C. The mixture was stirred at room temperature for 1.5 h. The solution of ClCH₂COOC₂H₅ (60 mg, 0.98 mmol) in DMF (1 ml) was added to the above solution at 0° C. The mixture was stirred at room temperature for 6 h. The mixture was quenched with ice-water and extracted with EA (2×30 ml). The organic layers were combined and washed with brine (10 ml), and filtered. The filtrate was concentrated and purified by flash chromatography using MeOH:DCM1:20 to give 13 (20 mg, 18.5%). Compound 13 was dissolved in methanol. The solution was heated to reflux for 3 h and evaporated to dryness. The residue was re-crystallization from 10% methanol in DCM to give compound 14 (18 mg, 95%). MS (m/z) (M⁺+H): 319. ¹H-NMR: (δ, DMSO, 400 Mz, ppm): 9.44 (s, 1H), 9.14 (d, 1H), 8.87 (dd, 2H), 8.64 (m, 1H), 8.32 (tt, 1H), 8.25 (d, 1H), 8.07 (dd, 1H), 7.58 (m, 1H), 5.68 (s, 2H), 3.70 (s, 3H).

### Example 3

### 3-methyl-8-(pyridin-3-yl)-3H-pyrazolo[3,4-c]quinoline (25) (1307)

### Example 4

### 2-methyl-8-(pyridin-3-yl)-2H-pyrazolo[3,4-c]quinoline (26) (1308)

To a mixture of compound 11 (100 mg, 0.41 mmol) and K₂CO₃ (84 mg, 0.61 mmol) in ethanol/toluene (1:1, 10 ml) was added CH₃I (58 mg, 0.41 mmol) at room temperature. The mixture was stirred at rt overnight. The solvent was removed under reduced pressure. The residue was purified by flash chromatography (MeOH:DCM1:20) to give Example 3 (15 mg, 14%) and Example 4 (10 mg, 13%).

### Example 3

MS (m/z) (M⁺+H): 261. ¹H-NMR: (δ, ppm, DMSO-d6, 400 Hz) 9.40 (s, 1H), 9.12 (d, 1H), 8.80 (d, 1H), 8.77 (s, 1H), 8.62 (q, 1H), 8.29 (d, 1H), 8.21 (d, 1H), 8.02 (dd, 1H), 7.55 (q, 1H), 4.28 (s, 3H).

### Example 4

MS (m/z) (M⁺+H): 261. ¹H-NMR: (δ, ppm, DMSO-d6, 400 Hz) 9.22 (s, 1H), 9.07 (d, 1H), 8.94 (s, 1H), 8.64 (d, 1H), 8.61 (q, 1H), 8.23 (t, 1H), 8.09 (d, 1H), 7.94 (dd, 1H), 7.53 (q, 1H), 4.26 (s, 3H).

### Example 5

### 3-ethyl-8-(pyridin-3-yl)-3H-pyrazolo[3,4-c]quinoline (27) (1309)

### Example 6

### 2-ethyl-8-(pyridin-3-yl)-2H-pyrazolo[3,4-c]quinoline (28). (1310)

To a mixture of compound 11 (100 mg, 0.41 mmol) and K₂CO₃ (84 mg, 0.61 mmol) in ethanol/toluene (5:1, 10 ml) was added BrCH₂CH₃ (44 mg, 0.41 mmol) at room temperature. The mixture was heated to 50° C. overnight. The solvent was removed by reduced pressure. The residue was purified by flash chromatography using MeOH:DCM1:20 to give compound Example 5 (10 mg, 9%) and Example 6 (8 mg, 7%).

### Example 5

MS (m/z) (M⁺+H): 275. ¹H-NMR: (δ, ppm, DMSO-d6, 400 Hz) 9.45 (s, 1H), 9.13 (d, 1H), 8.82 (d, 1H), 8.80 (d, 1H), 8.62 (q, 1H), 8.31 (m, 1H), 8.21 (d, 1H), 8.02 (dd, 1H), 7.55 (m, 1H), 4.68 (q, 2H), 1.49 (t, 3H).

### Example 6

MS (m/z) (M⁺+H): 275. ¹H-NMR: (δ, ppm, DMSO-d6, 400 Hz) 9.24 (s, 1H), 9.08 (d, 1H), 9.03 (s, 1H), 8.66 (d, 1H), 8.61 (d, 1H), 8.25 (d, 1H), 8.10 (d, 1H), 7.95 (dd, 1H), 7.54 (q, 1H), 4.56 (q, 2H),1.56 (t, 3H).

### Example 7

### N-methyl-N-(4-(8-(pyridin-3-yl)-3H-pyrazolo[3,4-c]quinolin-1-yl)phenyl)acetamide. (1311)

A mixture of compound 12 (100 mg, 0.31 mmol), PdCl₂(dppf) (11.3 mg, 0.015 mmol), 4-(N-methylacetamido)phenylboroic acid (60.6 mg, 0.31 mmol), and K₂CO₃ (127 mg, 0.92 mmol) in DMF (3 ml) and H₂O (1.5 ml) was heated to 110° C. under N₂ overnight. The solvent was removed under reduced pressure. The residue was purified by flash chromatography using MeOH:DCM1:20 to give title compound (9 mg, 7.4 MS (m/z) (M⁺+H): 394. ¹H-NMR: (δ, ppm, DMSO-d6, 300 Hz) 14.28 (s, 1H), 9.33 (s, 1H), 8.84 (d, 1H), 8.59 (d, 1H), 8.33 (t, 2H), 8.05 (t, 2H), 7.91 (d, 2H), 7.62 (d, 2H), 7.50 (m, 1H), 3.27 (s, 3H), 1.91 (s, 3H).

### Example 8

### 4-(8-(pyridin-3-yl)-3H-pyrazolo[3,4-c]quinolin-1-yl)benzonitrile (1312)

A mixture of compound 12 (100 mg, 0.31 mmol), 4-cyanophenylboronic acid (45 mg, 0.31 mmol), K₂CO₃ (127 mg, 0.92 mmol) and PdCl₂(dppf) (11.3 mg, 0.015 mmol) in DMF (3 ml) and H₂O (1.5 ml) was heated to 110° C. under N₂ overnight. The solvent was removed under reduced pressure. The residue was purified by flash chromatography using MeOH:DCM1:20 to give target compound (14 mg, 13%). 13%. MS (m/z) (M⁺+H): 348. ¹H-NMR: (δ, ppm, DMSO-d6, 300 Hz) 14.49 (s, 1H), 9.35 (s, 1H), 8.88 (d, 1H), 8.61 (m, 1H), 8.36 (s, 1H), 8.30 (d, 1H), 8.02-8.12 (m, 6H), 7.56 (m, 1H).

### Example 9

### tert-butyl 1-bromo-8-(pyridin-3-yl)-3H-pyrazolo[3,4-c]quinoline-3-carboxylate (17)

To a solution of compound 12 (100 mg, 0.31 mmol) and NaOH (18.5 mg, 0.46 mmol) in 1,4-dioxane (10 ml) and H₂O (10 ml) was added (Boc)₂O (80.5 mg, 0.37 mmol) at 0° C. The solution was stirred at room temperature overnight and filtered. The cake was dried by vacuum to give 17 (78 mg, 60%). MS (m/z) (M⁺+H): 425, 427.

### 2-(4-(8-(pyridin-3-yl)-3H-pyrazolo[3,4-c]quinolin-1-yl)phenyl)propan-2-ol (18) (1313)

A mixture of compound 17 (100 mg, 0.24 mmol), K₂CO₃ (97 mg, 0.71 mmol), PdCl₂(dppf) (8.6 mg, 0.012 mmol) and 2-(4-(4,4,5,5-tetramethyl-1,3,2-dioxaborolan-2-yl) phenyl)propan-2-ol (124 mg, 0.47 mmol) in DMF (6 ml) and H₂O (3 ml) was heated to 150° C. under N₂ overnight. The solvent was removed under reduced pressure. The residue was purified by flash chromatography using MeOH:DCM1:20 to give target compound (15 mg, 17%). MS (m/z) (M⁺+H): 381. ¹H-NMR: (δ, ppm, DMSO-d6, 400 Hz) 9.25 (s, 1H), 8.74 (s, 1H), 8.52 (q, 1H), 8.41 (s, 1H), 8.05 (d, 1H), 7.97 (d, 1H), 7.78 (s, 4H), 7.51 (q, 1H), 1.63 (s, 6H),

### Example 10

### 2-(4-(8-(pyridin-3-yl)-3H-pyrazolo[3,4-c]quinolin-1-yl)phenyl)acetonitrile (19) (1314)

A mixture of compound 17 (100 mg, 0.24 mmol), K₂CO₃ (97 mg, 0.71 mmol), PdCl₂(dppf) (8.6 mg, 0.012 mmol) and 2-(4-(4,4,5,5-tetramethyl-1,3,2-dioxaborolan-2-yl) phenyl)acetonitrile (114 mg, 0.47 mmol) in DMF (6 ml) and H₂O (3 ml) was heated to 150° C. under N₂ overnight. The solvent was removed under reduced pressure. The residue was purified by flash chromatography using MeOH:DCM1:20 to give target compound (25 mg, 30%). MS (m/z) (M⁺+H): 362. ¹H-NMR: (δ, ppm, DMSO-d6, 300 Hz) 14.3 (s, 1H), 9.35 (s, 1H), 8.90 (s, 1H), 8.62 (t, 1H), 8.31 (s, 1H), 8.29 (d, 1H), 8.06 (m, 2H), 7.93(d, 2H), 7.66 (s, 1H), 7.63 (s, 1H), 7.53 (q, 1H), 4.24 (s, 2H).

### Example 11

### 1,8-di(pyridin-3-yl)-3H-pyrazolo[3,4-c]quinoline (20) (1315)

A mixture of 17 (100 mg, 0.24 mmol), K₂CO₃ (97 mg, 0.71 mmol), PdCl₂(dppf) (8.6 mg, 0.012 mmol) and 3-(4,4,5,5-tetramethyl-1,3,2-dioxaborolan-2-yl)pyridine (96 mg, 0.47 mmol) in DMF (6 ml) and H₂O (3 ml) was heated to 150° C. under N₂ overnight. The solvent was removed under reduced pressure. The residue was purified by flash chromatography using MeOH:DCM1:20 to give target compound (30 mg, 40%). MS (m/z) (M⁺+H): 346. ¹H-NMR: (δ, ppm, DMSO-d6, 300 Hz) 14.5 (s, 1H), 9.38 (s, 1H), 9.09 (s, 1H), 8.87 (d, 1H), 8.81 (d, 1H), 8.63 (d, 1H), 8.06 (m, 3H), 7.71 (q, 1H), 7.55 (q, 1H).

### Example 12

### tert-butyl 5-(8-(pyridin-3-yl)-3H-pyrazolo[3,4-c]quinolin-1-yl)pyridin-3-yl carbamate (21) (1316)

A mixture of compound 17 (100 mg, 0.24 mmol), K₂CO₃ (97 mg, 0.71 mmol), PdCl₂(dppf) (8.6 mg, 0.012 mmol) and tert-butyl-5-(4,4,5,5-tetramethyl-1,3,2-dioxaborolan-2-yl)pyridine-3-ylcarbamate (150.6 mg, 0.47 mmol) in DMF (6 ml) and H₂O (3 ml) was heated to 150° C. under N₂ overnight. The solvent was removed under reduced pressure. The residue was purified by flash chromatography using MeOH:DCM1:20 to give target compound (15 mg, 15%). MS (m/z) (M⁺+H): 439. ¹H-NMR: (δ, ppm, DMSO-d6, 300 Hz) 14.50 (s, 1H), 9.96 (s, 1H), 9.38 (s, 1H), 8.90 (d, 1H), 8.73 (d, 1H), 8.66 (s, 1H), 8.62 (q, 1H), 8.46 (d, 1H), 8.32 (d, 1H), 8.08 (m, 2H), 7.51 (q, 1H), 1.47 (s, 9H).

### Example 13

### N-(4-(8-(pyridin-3-yl)-3H-pyrazolo[3,4-c]quinolin-1-yl)phenyl)methanesulfonamide (22) (1317)

Similar procedure described for Example 12. 9%. MS (m/z) (M⁺+H): 416. ¹H-NMR: (δ, ppm, MeOH-D4, 400 Hz) 10.05 (s, 1H), 9.26 (s, 1H), 8.94 (m, 2H), 8.72 (s, 1H), 8.53 (d, 1H), 8.39 (d, 1H), 8.26 (t, 1H), 7.93 (d, 2H), 7.60 (d, 2H), 3.12 (s, 3H).

### Example 14

### 1-(4-(8-(pyridin-3-yl)-3H-pyrazolo[3,4-c]quinolin-1-yl)phenyl)pyrrolidin-2-one (23) (1318)

Similar procedure described for Example 12. 13%. MS (m/z) (M⁺+H): 406. ¹H-NMR: (δ, ppm, DMSO-d6, 400 Hz): 9.29 (s, 1H), 8.85 (s, 1H), 8.58 (t, 1H), 8.46 (s, 1H), 8.22 (d, 1H), 8.06 (d, 1H), 7.96 (s, 1H), 7.90 (q, 4H), 7.50 (t, 1H), 3.92 (t, 2H), 2.55 (t, 2H), 2.12 (q, 2H).

### Example 15

### N-methyl-N-(4-(8-(pyridin-3-yl)-3H-pyrazolo[3,4-c]quinolin-1-yl)phenyl)methanesulfonamide (24) (1319)

Similar procedure described for Example 12. 25%. MS (m/z) (M⁺+H): 430. ¹H-NMR: (δ, ppm, MeOH-D4, 400 Hz) 10.06 (s, 1H), 9.29 (s, 1H) 8.95 (d, 1H), 8.90 (d, 1H), 8.55 (d, 1H), 8.54 (dd, 1H), 8.43 (dd, 1H), 8.27 (m, 1H), 8.25 (d, 2H), 7.82 (d, 2H), 3.45 (s, 3H), 3.03 (s, 3H).

### Example 16

### 6-Bromo-4-methylquinolin-3-amine (35)

To a solution of 6-bromo-4-methyl-3-nitroquinoline 7 (3.3 g, 1 eq) in AcOH (500 mL), Fe power (3 eq) was added. The reaction mixture was stirred at room temperature for 30 min. The solid was removed by filtration, and the filtrate was concentrated in vacuo to give a crude product., which was dissolved in water and extracted with ethyl acetate, dried over MgSO₄, filtered, and concentrated in vacuo to afford compound 35 (2.4 g: 85%). MS (m/z) (M⁺+H): 237, 239.

### 8-Bromo-3H-pyrazolo[3,4-c]quinoline (36) (1320)

To a 25 mL round-bottom flask was charged with 6-bromo-4-methylquinolin-3-amine (35) (2.4 g, 1 eq), (Ac)₂O (3 eq), AcOK (5 eq) in 15 mL of toulene. The solution was heated at 60° C. for 2 h. To the mixture t-butyl nitrite (3 eq) was added. The reaction mixture was stirred at 85° C. for 8 h under N₂ protection. The mixture was diluted with water (10 mL) and extracted with EA (3×20 mL). Organic layer was washed with brine, dried over Na₂SO₄, filtered, and concentrated. The residue was purified by column chromatography to give 1-(8-bromo-3H-pyrazolo[3,4-c]quinolin-3-yl)ethanone. (1.9 g, 65%)

A mixture of 1-(8-bromo-3H-pyrazolo[3,4-c]quinolin-3-yl)ethanone (1.5 g, 1 eq), K₂CO₃ (3 eq) in CH₃OH (50 mL) was refluxed for 2 h. The solvent was removed in vacuo. The residue was diluted with water (20 mL) and extracted with DCM (3×30 mL). Organic layer was washed with brine, dried over Na₂SO₄, filtered, and concentrated. The residue was purified by column chromatography to give 36 (890 mg, 70%). MS (m/z) (M⁺+H): 248, 250. ¹H-NMR: (δ, ppm, DMCO-d6, 400 MHz): 9.32 (s, 1H), 8.79 (s, 1H), 8.61 (d, 1H), 8.08 (d, 1H), 7.79-7.76 (dd, 1H).

### Example 17

### N-(5-(3H-pyrazolo[3,4-c]quinolin-8-yl)pyridin-3-yl)acetamide (31)

To a mixture of compound 44 (350 mg, 1 eq), DMSO (10 eq), KOtBu (5 eq) in THF (10 mL), O₂ stream was bubble for 10 min. The solvent was removed in vacuo. The residue was diluted with water (20 mL) and extracted with DCM (3×30 mL). Organic layer was washed with brine, dried over Na₂SO₄, filtered, and concentrated. The residue was purified by column chromatography to give 31 (188 mg, 60%). MS (m/z) (M⁺+H): 304. ¹H-NMR: (δ, ppm, CDCl3, 400 MHz): 13.20 (s, 1H), 9.76 (s, 1H), 9.52 (s, 1H), 8.75 (m, 4H), 8.26 (d, 1H), 2.17 (s, 3H).

### N-(5-(1-bromo-3H-pyrazolo[3,4-c]quinolin-8-yl)pyridin-3-yl)acetamide (32)

A mixture of compound 31 (188 mg, 1 eq), NBS (1.1 eq) in CH₃CN (20 mL) was refluxed for 0.5 h. The solvent was removed in vacuo. The residue was diluted with water (20 mL) and extracted with DCM (3×30 mL). Organic layer was washed with brine, dried over Na₂SO₄, filtered, and concentrated. The residue was purified by column chromatography to give 45 (77 mg, 70%).

### Example 18

### 3-Benzyl-8-bromo-3H-pyrazolo[3,4-c]quinoline (43) and 2-Benzyl-8-bromo-2H-pyrazolo[3,4-c]quinoline (43')

A mixture of compound 36 (700 mg, 1 eq), (bromomethyl)benzene (1 eq), K₂CO₃ (3 eq) in EtOH (50 mL) was refluxed for 2 h. The solvent was removed in vacuo. The residue was diluted with water (50 mL) and extracted with DCM (3×50 mL). Organic layer was washed with brine, dried over Na₂SO₄, filtered, and concentrated. The residue was purified by column chromatography to give 43 (500 mg, 52%) and 43′ (300 mg, 31%).

### N-(5-(3-benzyl-3H-pyrazolo[3,4-c]quinolin-8-yl)pyridin-3-yl)acetamide (44) (1322)

To a 25 mL round-bottom flask was charged with N-(5-bromopyridin-3-yl)acetamide (500 mg, 1 eq), Bis(pinacolato)diboron (1.1 eq), PdCl₂(dppf) (0.05 eq), AcOK (3 eq) in 15 mL of dioxane. The mixture was thoroughly degassed by alternately connecting the flask to vacuum and nitrogen. The solution was heated at 85° C. for 8 h. The solvent was removed in vacauo to afford a mixture containing N-(5-(4,4,5,5-tetramethyl-1,3,2-dioxaborolan-2-yl)pyridin-3-yl)acetamide. To the mixture, compound 43 (1 eq□, 2 M K₂CO₃ (5 eq) and Pd(PPh₃)₄ (10 mg) and DMF (10 mL) was added. The reaction mixture was stirred at 155° C. for 8 h under N₂ protection. The mixture was diluted with water (10 mL) and extracted with DCM (3×20 mL). Organic layer was washed with brine, dried over Na₂SO₄, filtered, and concentrated. The residue was purified by column chromatography to give compound 44 (377 mg, 65%(two step)). 394. ¹H-NMR: (δ, ppm, CDCl3, 400 MHz): 10.33 (s, 1H), 9.50 (s, 1H), 8.86 (s, 1H), 8.78-8.75 (m, 3H), 8.45 (s, 1H), 8.24-8.22 (d, 1H), 7.94-7.91 (d, 1H), 7.32-7.27 (m, 5H), 5.92 (s, 2H), 2.11 (s, 3H).

### Example 19

### N-(5-(3-benzyl-1-bromo-3H-pyrazolo[3,4-c]quinolin-8-yl)pyridin-3-yl)acetamide (46) (1323)

A mixture of compound 32 (130 mg, 1 eq), (bromomethyl)benzene (1 eq), K₂CO₃ (3 eq) in EtOH (20 mL) was refluxed for 2 h. The solvent was removed in vacuo. The residue was diluted with water (20 mL) and extracted with DCM (3×30 mL). Organic layer was washed with brine, dried over Na₂SO₄, filtered, and concentrated. The residue was purified by column chromatography to give 46 (127 mg, 79%). MS (m/z) (M⁺+H): 372, 374. ¹H-NMR: (δ, ppm, CDCl3, 400 MHz): 10.36 (s, 1H), 9.58 (s, 1H), 8.99 (s, 1H), 8.79 (s, 1H), 8.70 (s, 1H), 8.47 (s, 1H), 8.30 (s, 1H), 8.04 (m, 5H), 5.92 (s, 2H), 2.11 (s, 3H).

### Example 20

### N-(5-(2-benzyl-2H-pyrazolo[3,4-c]quinolin-8-yl)pyridine-3-yl)acetamide (48) (1324)

To a 25 mL round-bottom flask was charged N-(5-bromopyridin-3-yl)acetamide (500 mg 1 eq), bis(pinacolato)diboron (1.1 eq), PdCl₂(dppf) (0.05 eq), AcOK (3 eq) in 15 mL of dioxane. The mixture was thoroughly degassed by alternately connecting the flask to vacuum and nitrogen. The solution was heated at 85° C. for 8 h. The solvent was removed in vacauo. To the mixture compound 43′ (1 eq), 2 M K₂CO₃(5 eq) and PdCl₂(dppf) (10 mg) and DMF (10 mL) was added. The reaction mixture was stirred at 155° C. for 8 h under N₂ protection. The mixture was diluted with water (10 mL) and extracted with DCM (3×20 mL). Organic layer was washed with brine, dried over Na₂SO₄, filtered, and concentrated. The residue was purified by column chromatography to give compound 48 (226 mg, 65% for two steps). MS (m/z) (M⁺+H): 394. ¹H-NMR: (δ, CDCl₃, 400 MHz, ppm): 9.34 (s, 1H), 8.70 (s, 1H), 8.59 (s, 1H), 8.49 (s, 1H), 8.32 (s, 1H), 8.19 (m, 2H), 7.81 (d, 1H), 7.41-7.36 (m, 5H), 5.69 (s, 2H), 2.27 (s, 3H).

### Example 21

### N-(5-(3-benzyl-1-(4-cyanophenyl)-3H-pyrazolo[3,4-c]quinolin-8-yl)pyridin-3-yl)acetamide (47) (1325)

To a 25 mL round-bottom flask was charged with compound 46 (70 mg, 1 eq, 4-cyanophenylboronic acid (1.1 eq), 2 M K₂CO₃ (5 eq), PdCl₂(dppf) (10 mg) and DMF (10 mL). The mixture was thoroughly degassed by alternately connecting the flask to vacuum and nitrogen. The reaction mixture was stirred at 155° C. for 8 h under N₂ protection. The mixture was diluted with water (10 mL) and extracted with DCM (3×20 mL). Organic layer was washed with brine, dried over Na₂SO₄, filtered, and concentrated. The residue was purified by column chromatography to give compound 47 (40 mg, 60%). MS (m/z) (M⁺+H): 495. ¹H-NMR: (δ, ppm, CDCl3, 400 MHz): 9.12 (s, 1H), 8.69 (s, 1H), 8.58 (s, 1H), 8.39 (s, 1H), 8.30 (m, 3H), 8.04 (m, 5H), 7.36 (m, 6H), 5.85 (s, 2H), 2.31 (s, 3H).

### Example 23

### N-(5-(1-(pyridin-4-yl)-3H-pyrazolo[3,4-c]quinolin-8-yl)pyridin-3-yl)acetamide (34) tert-butyl (1327)

To a solution of 32 (100 mg, 0.23 mmol) and TEA (0.06 ml, 0.45 mmol) in dry DCM (4 ml) was added (Boc)₂O (74 mg, 0.34 mmol) at 0° C. The solution was stirred at rt overnight and filtered. The solid was dried to give 8-(5-acetamidopyridin-3-yl)-1-bromo -3H-pyrazolo[3,4-c]quinoline-3-carboxylate, 33 (60 mg, 47%)

Example 20 (34) was followed general coupling procedure to provide 34. Yield: 59%. MS (m/z) (M⁺+H): 381. ¹H-NMR: (δ, ppm, MeOH-D4, 400 MHz): 9.28 (s, 1H), 8.84 (d, 2H), 8.59 (m, 2H), 8.51 (dd, 2H), 8.30 (d, 1H), 7.99 (m, 3H), 2.22 (s, 3H).

### Examples 22 and 24

### 1,8-Dibromo-3H-pyrazolo[3,4-c]quinoline (37), 3-Benzyl-1,8-dibromo-3H-pyrazolo[3,4-c]quinoline (38) and 2-Benzyl-1,8-dibromo-3H-pyrazolo[3,4-c]quinoline (39)

A mixture of compound 36 (6 g, 1 eq), NBS (1.1 eq) in CH₃CN:DMF (100 mL: 50 mL) was heated at 80° C. for 0.5 h. To the mixture (37), (bromomethyl)benzene (2 eq), K₂CO₃ (5 eq) was added. The mixture was heated at 80° C. for 0.5 h. the mixture was filtered and the filtrate was concentrated. The residue was purified by column chromatography to give 38 (4 g, two step: 88%) and 39 (100 mg). MS (m/z) (M⁺+H) for 38 and 39: 418.

### Examples 22

### 3-Benzyl-8-bromo-1-morpholino-3H-pyrazolo[3,4-c]quinoline (40) (1326)

### Examples 24

### 3-Benzyl-1,8-dimorpholino-3H-pyrazolo[3,4-c]quinoline (41) (1328)

A mixture of compound 38 (500 mg, 1 eq), morpholine (10 eq), NaO^{t}Bu (7 eq), Pd(AcO)₂ (0.1 eq), and BINAP (0.1 eq) in toluene (20 mL) was refluxed for 8 h under N₂ protection. The solvent was removed in vacuo. The residue was diluted with water (20 mL) and extracted with DCM (3×30 mL). Organic layer was washed with brine, dried over Na₂SO₄, filtered, and concentrated. The residue was purified by column chromatography to give 40 (150 mg, 30%) and 41 (300 mg, 56%).

### Examples 22

MS (m/z) (M⁺+H): 423, 425. ¹H-NMR: (δ, ppm, CDCl3, 400 MHz): 8.82 (s, 1H), 8.23 (s, 1H), 8.08 (m, 1H), 7.36 (m, 6H), 5.71 (s, 2H), 3.96 (m, 4H), 3.41 (m, 4H). Examples 24: MS (M/Z) M++H): 430. ¹H-NMR: (δ, ppm, CDCl3, 400 MHz): 8.76 (s, 1H), 8.06 (d, 1H), 7.63 (s, 1H), 7.31 (m, 6H), 5.62 (s, 2H), 4.00-3.92 (m, 8H), 3.41-3.34 (m, 8H).

### Example 25

### 1,8-Dimorpholino-3H-pyrazolo[3,4-c]quinoline (42) (1329)

To a mixture of compound 41 (70 mg, 1 eq), DMSO (10 eq), KOtBu (5 eq) in THF (10 mL), O₂ beam was bubbled for 10 min. The solvent was removed in vacuo. The residue was diluted with water (20 mL) and extracted with DCM (3×20 mL). Organic layer was washed with brine, dried over Na₂SO₄, filtered, and concentrated. The residue was purified by column chromatography to give 42 (40 mg, 70%). MS (m/z) (M⁺+H): 340. ¹H-NMR: (δ, ppm, CDCl3, 400 MHz): 8.92 (s, 1H), 8.01 (d, 1H), 7.70 (s, 1H), 7.41 (m, 1H), 3.95-3.87 (m, 8H), 3.39-3.27 (m, 8H).

V. Preparation of Intermediates and Final Targets in Other Scaffolds

Compounds with various fused tri-membered core derivatives were classified as other Scaffolds. The final compounds were summarized in Table V.

TABLE V

| Cpd | | | | MS | |
|---|---|---|---|---|---|
| No | Ex | Structure | MF/MW | (M⁺ + H) | IUPAC |
| 1330 | 1 | 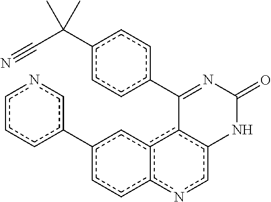 | C26H19N5O/ 417.5 | 419 | 2-methyl-2-(4-(3-oxo-9-(pyridin- 3-yl)-3,4-dihydropyrimido[4,5- c]quinolin-1-yl) phenyl)propanenitrile |
| 1331 | 2 | 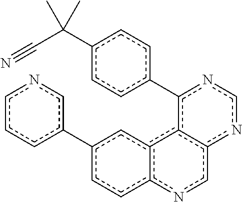 | C26H19N5/ 401.5 | 402.2 | 2-methyl-2-(4-(9-(pyridin-3- yl)pyrimido[4,5-c]quinolin-1- yl)phenyl)propanenitrile |
| 1332 | 3 | 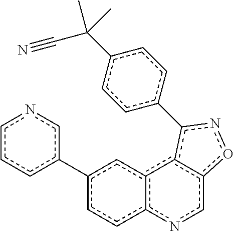 | C25H18N4O/ 390.4 | 391.1 | 2-methyl-2-(4-(8-(pyridin-3- yl)isoxazolo[5,4-c]quinolin-1- yl)phenyl)propanenitrile |
| 1333 | 4 | 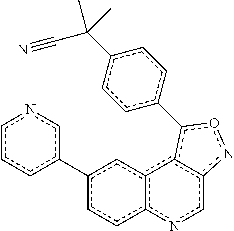 | C25H18N4O/ 390.4 | 391 | 2-methyl-2-(4-(8-(pyridin-3- yl)isoxazolo[3,4-c]quinolin-1- yl)phenyl)propanenitrile |
| 1334 | 5 | 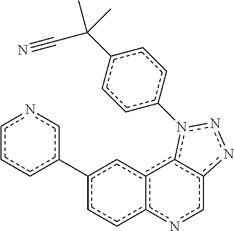 | C24H18N6/ 390.4 | 391 | 2-methyl-2-(4-(8-(pyridin-3- yl)-1H-[1,2,3]triazolo[4,5- c]quinolin-1- yl)phenyl)propanenitrile |
| 1335 | 6 | 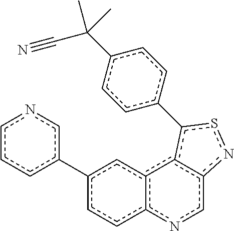 | C25H18N4S/ 406.5 | 407 | 2-methyl-2-(4-(8-(pyridin-3- yl)isothiazolo[3,4-c]quinolin-1- yl)phenyl)propanenitrile |
| 1336 | 7 | 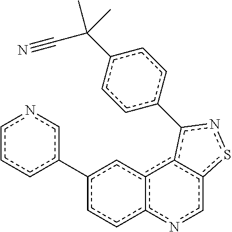 | C25H18N4S/ 406.5 | 407 | 2-methyl-2-(4-(8-(pyridin-3- yl)isothiazolo[5,4-c]quinolin-1- yl)phenyl)propanenitrile |
| 1026 | 8 | 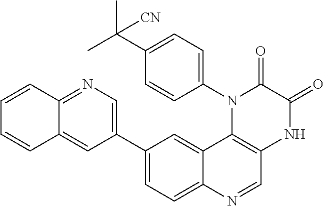 | C30H21N5O2/ 483 | 484 | 2-(4-(2,3-dioxo-9-(quinolin-3- yl)-3,4-dihydropyrazino[2,3- c]quinolin-1(2H)-yl)phenyl)-2- methylpropanenitrile |

Synthetic Procedures for Preparing the Compounds in Table V.

All compounds in this Table were synthesized from unique approaches and their synthetic Schemes and procedures were listed following.

### Example 1

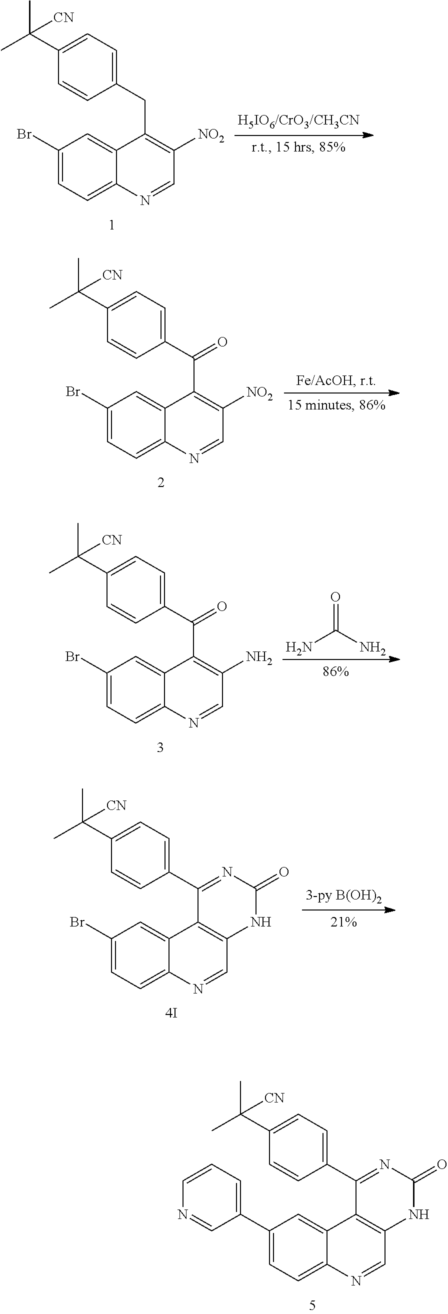

### 2-(4-(6-bromo-3-nitroquinoline-4-carbonyl)phenyl)-2-methylpropanenitrile (2)

To a solution of 2-(4-((6-bromo-3-nitroquinolin-4-yl)methyl)phenyl)-2-methyl propanenitrile (1, 1 g, 2.4 mmol, 1 eq) in MeCN (50 ml), was slowly added 0.1M CrO₃/MeCN solution (2 mL) and H₅IO₆ (1.6 g, 7.2 mmol, 3 eq) under vigorous stirring over 3 hours. After 12 hr an additional 0.9 mL of 0.1 M CrO₃/MeCN solution and H₅IO₆ (0.56 g) was added. The mixture was allowed to stir for an additional 3 hr before a solution of 5% sodium thiosulfate solution was added. The mixture was extracted with EtOAc. The organic phased was washed with aqueous NaHCO₃, dried with MgSO₄ and evaporated to afford 2 as yellow solid (0.85 g, 85%). MS (m/z) (M⁺+H): 424.

### 2-(4-(3-amino-6-bromoquinoline-4-carbonyl)phenyl)-2-methylpropanenitrile (3)

A mixture of 2 (400 mg, 0.94 mmol, 1 eq) and Fe (528 mg, 9.4 mmol, 10 eq) in AcOH (25 mL) was stirred for 30 min at room temperature and monitored by TLC or MS. The solvent was evaporated. Water was added and the pH was adJ=usted to 7 with aqueous K₂CO₃. The mixture was extracted with EtOAc, dried over MgSO₄, evaporated to afford 3 (320 mg, 86%). MS (m/z) (M⁺+H): 394.

### 2-(4-(9-bromo-3-oxo-3,4-dihydropyrimido[4,5-c]quinolin-l-yl)phenyl)-2-methylpropanenitrile (4)

A mixture of 3 (100 mg, 0.25 mmol, 1 eq) and urea (150 mg, 2.5 mmol, 10 eq) was heated at 200° C. for 1 h. After cooling to room temperature, the mixture was washed with water, and EtOAc, dried in vacuum. The residue was re-crystallized in EtOH to afford 4 (90 mg, 86%), MS (m/z) (M⁺+H): 419. ¹H-NMR (ppm, DMSO-d6, 400 MHz): 11.20 (s, br, 1H), 8.94 (s, 1H), 7.86 (d, 1H, J=7.7 Hz), 7.74-7.72 (d, 2H, J=8.0 Hz), 7.54-7.52 (m, 2H), 7.34-7.30 (m, 2H), 1.78(s, 6H).

2-methyl-2-(4-(3-oxo-9-(pyridin-3-yl)-3,4-dihydropyrimido[4,5-c]quinolin-1-yl)phenyl)propanenitrile (5) (1330)

To a solution of 4 (84 mg) in DMF (4 mL) was added 3-pyridylboronic acid (112 mg, 1 mmol), 1M Na₂CO₃ (100 mg, 0.6 mmol, in 0.6 mL water) and Pd(PPh₃)₄ (22 mg, 0.1 mmol). The reaction mixture was stirred under microwave for 15 min at 100° C. The mixture was diluted with water (10 mL) and extracted with DCM (3×20 mL). Organic layer was washed with brine, dried over Na₂SO₄, filtered, and concentrated. The residue was purified by column chromatography (DCM:Methanol 80:1 to 60:1) to give 5 (18 mg, 21%) as a light yellow solid. MS (m/z) (M⁺+H): 419; ¹H-NMR (δ, ppm, DMSO-d6, 400 MHz): 10.37 (s, 1H), 8.72 (s, 1H), 8.54 (d, 1H, J=2.4 Hz), 8.30 (s, 1H), 8.22 (s, 1H), 8.01 (d, 1H, J=10.2 Hz), 7.75 (m, 2H), 7.61 (d, 1H), 7.51 (m, 3H), 7.41 (m, 1H), 1.66 (s, 6H).

### Example 2

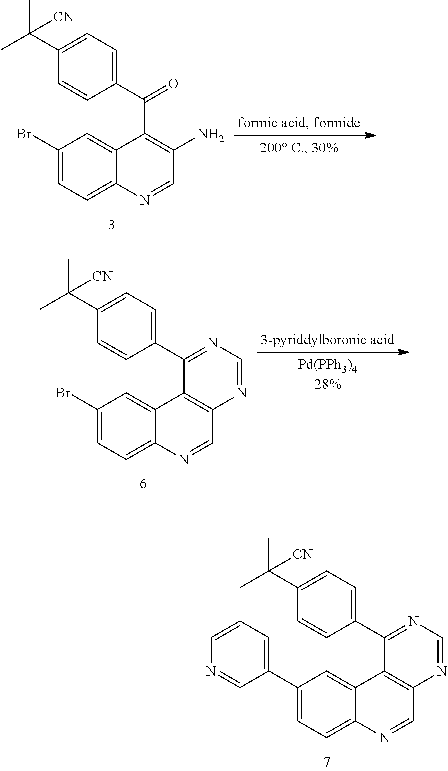

### 2-(4-(9-bromopyrimido[4,5-c]quinolin-1-yl)phenyl)-2-methylpropanenitrile(6)

A solution of 2 (100 mg, 0.25 mmol, 1 eq) in formic acid (0.25 mL) and formamide (1 mL) was boiled at 170° C. for 30 min. After cooled, the mixture was purified by flash chromatography to afford 30 mg of 6: 30%. MS (m/z) (M⁺+H): 403. ¹H-NMR (δ, ppm, DMSO-d6, 400 MHz): 9.63 (s, 1H), 9.54 (s, 1H), 8.49 (s, 2H), 8.14-8.11 (d, 1H, J=9.0 Hz), 7.95 (d, 1H, J=1.91), 7.83-7.80 (d, 2H, J=8.4 Hz), 7.71-7.69 (d, 2H, J=8.2 Hz), 7.44 (s, 1H), 1.73 (s, 6H).

### 2-methyl-2-(4-(9-(pyridin-3-yl)pyrimido[4,5-c]quinolin-1-yl)phenyl)propanenitrile (7) (1331)

To a solution of 6 (60 mg, 0.2 mmol) in DMF (4 mL) was added 3-pyridylboronic acid (112 mg), 1M Na₂CO₃ (100 mg, 0.6 mmol, in 0.6 mL water) and Pd(PPh₃)₄ (22 mg, 0.1 mmol). The reaction mixture was stirred under microwave for 30 min at 105° C. The mixture was diluted with water (10 mL) and extracted with DCM (3×20 mL). Organic layer was washed with brine, dried over Na₂SO₄, filtered, and concentrated. The resulted residue was purified by column chromatography (DCM:Methanol 80:1 to 60:1) to give 7 (11 mg, 28%, 95% purity) as a light yellow solid. MS (m/z) (M⁺+H): 402.2. ¹H-NMR (d-DMSO, 400 MHz): 9.64 (s, 1H), 9.56 (s, 1H), 8.58 (s, 1H), 8.40 (s, 1H), 8.32 (d, 1H), 8.19 (d, 1H), 7.83 (m, 6H), 7.44 (m, 1H), 1.84 (s, 6H).

### Example 3

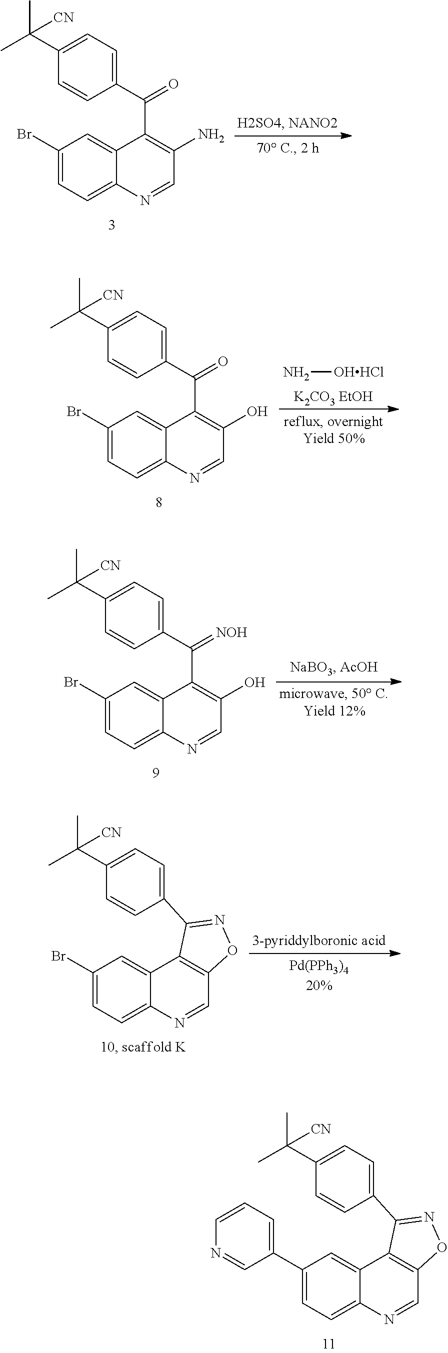

### 2-(4-(6-bromo-3-hydroxyquinoline-4-carbonyl)phenyl)-2-methylpropanenitrile (8)

To a suspension of 3, 2-(4-(3-amino-6-bromoquinoline-4-carbonyl)phenyl)-2-methyl propanenitrile (10 mg, 0.025 mmol) in H₂SO₄ (2.0 N, 1 mL) was added 1-INO₂ (5 mg, 01 mmol) at 0° C. After stirring for 0.5 h at room temperature and for 1 h at 70° C., a solution of NH₄OH was added. Filtered and washed some water to give 8 (10 mg. 100%) as a light yellow solid. MS (m/z) (M⁺+H): 395, 397.

### 2-(4-((6-bromo-3-hydroxyquinolin-4-yl)(hydroxyimino)methyl)phenyl)-2-methylpropanenitrile (9)

A mixture of 8 (100 mg) and hydroxylamine hydrochloride (200 mg) in EtOH was heated to reflux for 1 hour. The mixture was evaporated to dryness, washed with minimum amount of water and dried to give 9 (49 mg, 50%). MS (m/z) (M⁺+H): 410, 412. ¹H-NMR (δ, ppm, DMSO-d6, 300 MHz): 9.64 (s, 1H), 8.25-8.23 (d, 1H, J=8.8 Hz), 8.14 (s, 1H), 7.99-7.93 (m, 1H), 7.87-7.85 (d, 1H, J=8.3 Hz), 7.13 (s, 1H), 7.05 (s, 1H), 1.55 (s, 6H).

### 2-(4-(8-bromoisoxazolo[5,4-c]quinolin-1-yl)phenyl)-2-methylpropanenitrile (10)

To a solution of compound 9 (530 mg) in AcOH (50 mL), was added NaBO₃ (110 mg, 1 eq.) The mixture was refluxed for 3 h, to which was added water (50 ml) and the mixture was extracted with EA (3×50 ml). Organic layers were combined, washed with brine (50 ml), dried over Na₂SO₄, filtered and concentrated. Purification by flash chromatography (EA:PE, 1:10) to yield 10 (60 mg, 12%) as a light yellow solid. MS (m/z) (M⁺+H): 392, 394.

### (2-methyl-2-(4-(8-(pyridin-3-yl)isoxazolo[5,4-c]quinolin-1-yl)phenyl)propanenitrile(11) (1332)

To a solution of 10 (60 mg, 0.15 mmol) in DMF (5 mL) was added pyridin-3-ylboronic acid (37 mg, 0.3 mmol), Na₂CO₃ (38 mg, 0.45 mmol, in 0.3 mL water) and Pd(PPh₃)₄ (16 mg, 0.015 mmol). The reaction mixture was stirred under microwave at 108° C. for 20 min. The resulting mixture was diluted with water (20 mL), extracted with DCM (3×50 mL). Organic layers were combined, washed with brine, dried over Na₂SO₄, filtered, and concentrated in vacuo. The resulting residue was purified by column chromatography (DCM:Methanol 60:1 to 30:1) to give 11 (12 mg, 20%) as a light yellow solid. MS (m/z) (M⁺+H): 391.1. ¹H-NMR (δ, MeOH-d4, 400 MHz, ppm), 9.83 (s, 1H), 9.19 (s, 1H), 8.89 (d, 1H, J=5.09 Hz), 8.78 (d, 1H, J=8.22 Hz), 8.62 (d, 1H, J=1.96 Hz), 8.46 (d, 1H, J=8.60 Hz), 8.33-8.35 (m, 1H), 8.24-8.32 (m, 1H), 8.14-8.18 (m, 1H), 7.86-8.03 (m, 3H), 1.84 (s, 6H).

### Example 4

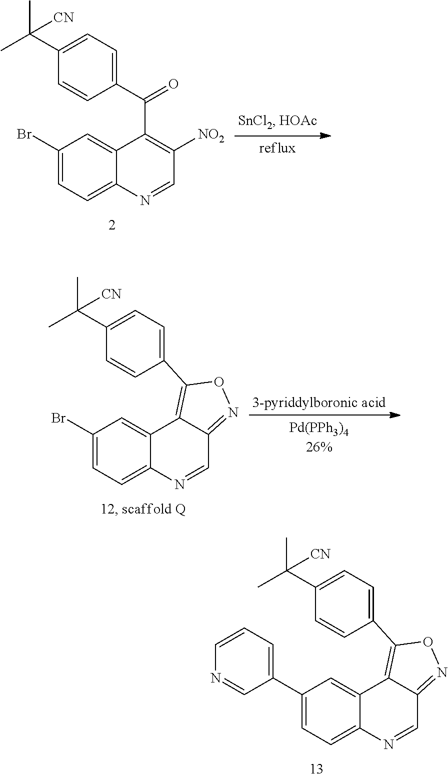

### 2-(4-(8-bromoisoxazolo[3,4-c]quinolin-1-yl)phenyl)-2-methylpropanenitrile (12)

A suspension of selenium dioxide (270 mg, 2.4 mmol, 5 eq) and 8 (200 mg, 0.48 mmol, 1 eq) in glacial acetic acid (15 ml) was heated at 100° C. for 3 hr and 120° C. for 2 hr. The solid was removed by filtration, and the filtrate was concentrated under reduced pressure. Water (5 ml) was added to the residue, and the mixture was neutralized with aqueous NaHCO₃, extracted with EtOAc, dried over MgSO₄, evaporated. Purification through column chromatography afforded 12 (40 mg: 21%). MS (m/z) (M⁺+H): 392, 394. ¹H-NMR (δ, CDCl3, 400MHz, ppm), 9.29 (s, 1H), 8.34 (s, 1H), 7.99-7.74 (m, 6H), 1.85 (s, 6H).

### 2-methyl-2-(4-(8-(pyridin-3-yl)isoxazolo[3,4-c]quinolin-1-yl)phenyl)propanenitrile (13) (1333)

To a solution of 12 (39 mg, 0.1 mmol) in DMF (4 mL) was added 3-pyridylboronic acid (25 mg, 0.2 mmol), 1M Na₂CO₃ (50 mg, 0.3 mmol, in 0.3 mL water) and Pd(PPh₃)₄ (11 mg). The reaction mixture was stirred under microwave for 15 min at 100° C. The mixture was diluted with water (10 mL) and extracted with DCM (3×20 mL). Organic layer was washed with brine, dried over Na₂SO₄, filtered, and concentrated. The residue was purified by column chromatography (DCM:Methanol 80:1 to 60:1) to give 13 (10 mg, 26%) as a light yellow solid. MS (m/z) (M⁺+H): 391. ¹H-NMR (δ, ppm, MeOH-D4, 400 MHz): δ 8.85 (s, 1H), 8.64 (s, 1H), 8.44 (s, 1H), 8.16 (dd, 1 H, J=1 4Hz, J=2 8 Hz), 7.98 (d, 1 H, J=9 Hz), 7.8-7.5 (m, 7H), 7.40-7.32 (m, 2H), 1.73 (s, 6H).

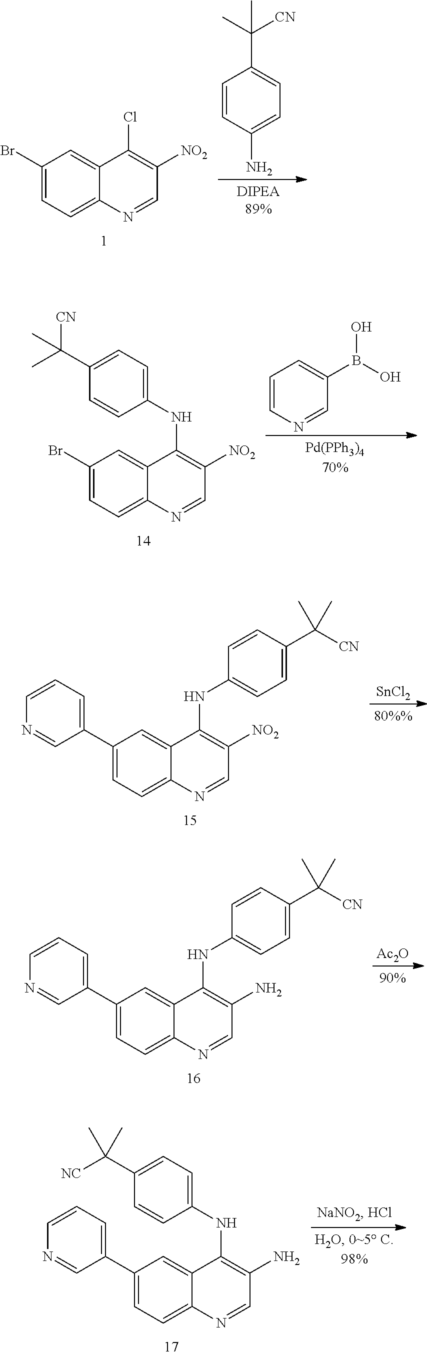

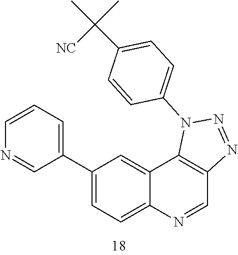

### 2-(4-(6-bromo-3-nitroquinolin-4-ylamino)phenyl)-2-methylpropanenitrile (14)

To a solution of compound 1 (4.3 g, 15 mmol) in acetic acid (100 mL) at 25° C., was added 2-(4-aminophenyl)-2-methylpropanenitrile (2.4 g, 15 mmol). The reaction mixture was stirred at 25° C. for 30 mins. The reaction mixture was quenched with ice-water and the solid was filtered, washed with 10% cold aqueous NaHCO₃ solution and cold water, and dried under vacuum to provide 14 (5.5 g, 89%) as a yellowish solid. MS (m/z) (M⁺+H): 411, 413; ¹H-NMR (400 Mz, DMSO-d6, ppm) 10.13 (s, 1H), 9.08 (s, 1H), 8.72 (s, 1H), 8.01 (d, 1H, J=8.55 Hz), 7.94 (d, 1H, J=8.97 Hz), 7.47 (d, 2H, J=8.55 Hz), 7.14 (d, 2H, J=8.55 Hz),1.68 (s, 6H).

### 2-methyl-2-(4-(3-nitro-6-(pyridin-3-yl)quinolin-4-ylamino)phenyl)propanenitrile (15)

A solution of 3-pyridylboronic acid (1.8 g, 15 mmol), compound 14 (4.1 g, 10 mmol), K₂CO₃ (4.14 g, 30 mmol), and Pd(PPh₃)₄(0.5 g, 0.5 mmol) in DMF (30 ml) was stirred at 90° C. under Ar overnight. The mixture was diluted with brine (5 ml) and EtOAc (30 ml). The layer was separated, and the aqueous layers was extracted with EtOAc (30 ml). The combined organic layers were washed with brine (5 ml), dried (Na₂SO₄), concentrated in vacuo, and purified by column chromatograph (ethyl acetate/petroleum ether1:8-1:2) to give compound 15 (2.9 g, 70%) as yellow solid. MS (m/z) (M⁺+H): 410.

### 2-(4-(3-amino-6-(pyridin-3-yl)quinolin-4-ylamino)phenyl)-2-methylpropanenitrile (16)

A mixture of compound 15 (2.9 g, 7.1 mmol), SnCl₂(4 g, 22 mmol) in MeOH (80 ml) was heated refluxed for 10 hr, then filter by suction. The filtrate was concentrated in vacuo and the crude product was purified by column chromatography (CH₂Cl₂/MeOH 1:25-1:10) to give 16 (2.14 g, 80%) as red solid. MS (m/z) (M⁺+H): 380.

### Example 6

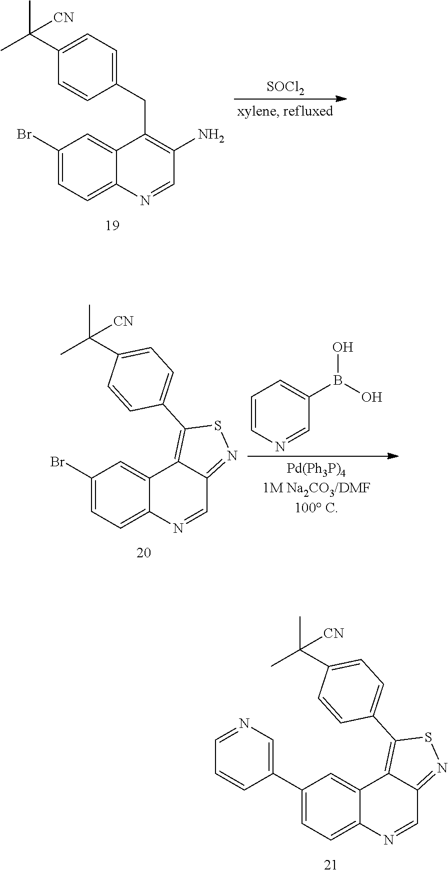

### 2-(4-(8-bromoisothiazolo[3,4-c]quinolin-1-yl)phenyl)-2-methylpropanenitrile (20)

To a solution of 19 (10 in I-1a), 2-(4-((3-amino-6-bromoquinolin-4-yl)methyl)phenyl)-2-methyl propanenitrile (380 mg, 1 mmol) in xylene (20 mL) was added SOCl₂ (240 mg, 2.0 mmol) at RT. The reaction mixture was refluxed overnight. The mixture was diluted with EA (50 mL), washed with saturated NaHCO₃ and brine, dried over Na₂SO₄, filtered, and concentrated. The residue was purified by column chromatography (EA:PE 1:10) to give 20 (125 mg, 30%) as a light yellow solid. MS (m/z) (M⁺+H): 408, 410; ¹H-NMR (δ, ppm, CDCl₃, 400 MHz): 9.29 (s, 1H), 7.97-8.02 (m, 2H), 7.74-7.77 (m, 3H), 7.65-7.72 (m, 2H), 1.86 (s, 6H).

### 2-methyl-2-(4-(8-(pyridin-3-yl)isothiazolo[3,4-c]quinolin-1-yl)phenyl)propanenitrile (21) (1335)

To a solution of 20 (82 mg, 0.21 mmol) in DMF (4 mL) was added 3-pyridylboronic acid (123 mg, 1 mmol), 1M Na₂CO₃ (100 mg, 0.6 mmol, in 0.6 mL water) and Pd(PPh₃)₄ (22 mg, 0.1 mmol). The reaction mixture was stirred under microwave for 15 min at 100° C. The mixture was diluted with water (10 mL), extracted with DCM (3×20 mL). Organic layer was washed with brine, dried over Na₂SO₄, filtered, and concentrated. The residue was purified by column chromatography (DCM:Methanol 80:1 to 60:1) to give 21 (20 mg, 25%) as a light yellow solid. MS (m/z) (M⁺+H): 407; ¹H-NMR (δ, ppm, DMSO-d6, 400 MHz): 9.34 (s, 1H), 8.64 (d, 1H, J=2.20 Hz), 8.57 (d, 1H, J=3.3 Hz), 8.20 (d, 1H, J=8.43 Hz), 8.10 (dd, 1H, J=12.2 Hz, J=28.43 Hz), 7.96 (d, 1H, J=1.83 Hz), 7.83 (dd, 2H, J=14.03 Hz, J=25.86 Hz), 7.53-7.64 (m, 3H), 7.43 (dd, 1H, J=14.76 Hz, J=27.70 Hz), 1.82 (s, 6H).

### Example 7

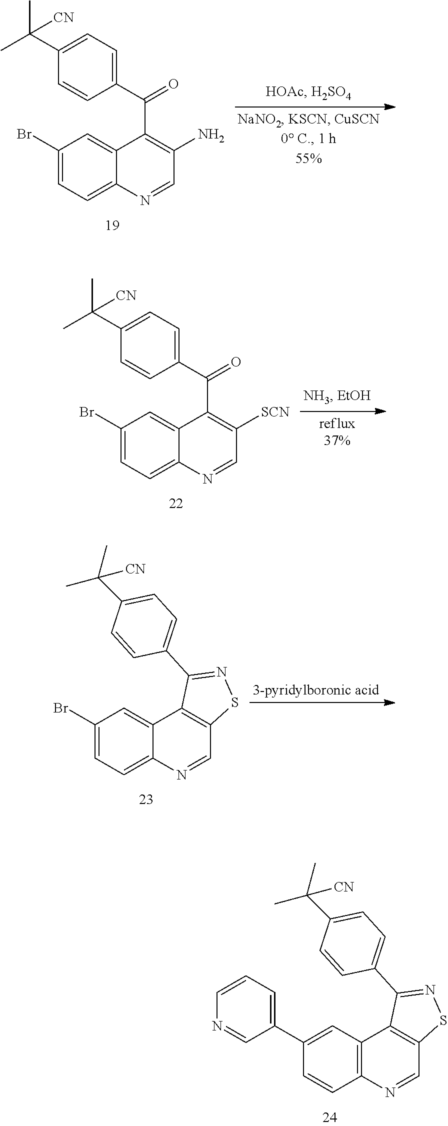

### 2-(4-(6-bromo-3-thiocyanatoquinoline-4-carbonyl)phenyl)-2-methylpropanenitrile (22)

To a solution of 19 (10 in II-1), 2-(4-(3-amino-6-bromoquinoline-4-carbonyl)phenyl)-2-methylpropanenitrile (1.6 g, 4.1 mmol) in AcOH (200 ml) and H₂SO₄ (60 ml), was added dropwise NaNO₂ (560 mg, 8.2 mmol) in water (10 ml)at −5° C. under stirring. The mixture was stirred for 0.5 hour at 0° C., and added into a solution of CuSCN (1.2 g, 9.8 mmol) and KSCN (1.56 g, 16.4 mmol) in water (500 ml). The mixture was stirred for 1 h at r.t. After filtered, the solid was washed with water (5×50 mL), and purified by silica gel column chromatography (EA:PE 4:1) to give 22 (880 mg, 50%) as a yellow solid. MS (m/z) (M⁺+H): 436, 438.

### 2-(4-(8-bromoisothiazolo[5,4-c]quinolin-1-yl)phenyl)-2-methylpropanenitrile (23)

Compound 22 (880 mg, 2.0 mmol) was dissolved in ammonia saturated DMF (100 ml). The solution was stirred overnight at RT, then was poured into water (200 ml), and extracted with DCM (2×50 mL). The organic phases were combined, washed with water (5×50 mL), dried over Na₂SO₄, filtered, and concentrated to afford 23 (300 mg, 32%) as yellow powder. MS (m/z) (M⁺+H): 408, 410.

### 2-methyl-2-(4-(8-(pyridin-3-yl)isothiazolo[5,4-c]quinolin-1-yl)phenyl)propanenitrile (24) (1336)

To a solution of 23 (Scaffold N) (82 mg, 0.2 mmol) in DMF (5 mL) was added pyridin-3-ylboronic acid (61 mg, 0.5 mmol), 1M Na₂CO₃ (64 mg, 0.6 mmol, in 0.60 mL water) and Pd(PPh₃)₄ (22 mg, 0.02 mmol). The reaction mixture was stirred under microwave for 30 min at 100° C. The reaction mixture was diluted with water (10 mL) and extracted with DCM (3×20 mL). Organic layers were combined, washed with brine, dried over Na₂SO₄, filtered, and concentrated. The residue was purified by column chromatography (silica gel, DCM:Methanol 150:1) to give 24 (30 mg, 37%) as a light yellow solid. ¹H-NMR: (δ, DMSO-d6, 400 MHz, ppm), 9.83 (s, 1H), 8.62 (d, 1H, J=2.35 Hz), 8.58 (dd, 1H, J=11.37 Hz, J=23.60 Hz), 8.35 (d, 1H, J=8.61 Hz), 8.18 (dd, 1H, J=11.95 Hz, J=26.93 Hz), 7.93 (d, 1H, J=1.76 Hz), 7.80-7.87 (m, 5H), 7.44 (dd, 1H, J=14.69 Hz, J=26.95 Hz), 1.83 (s, 6H).

### Example 8

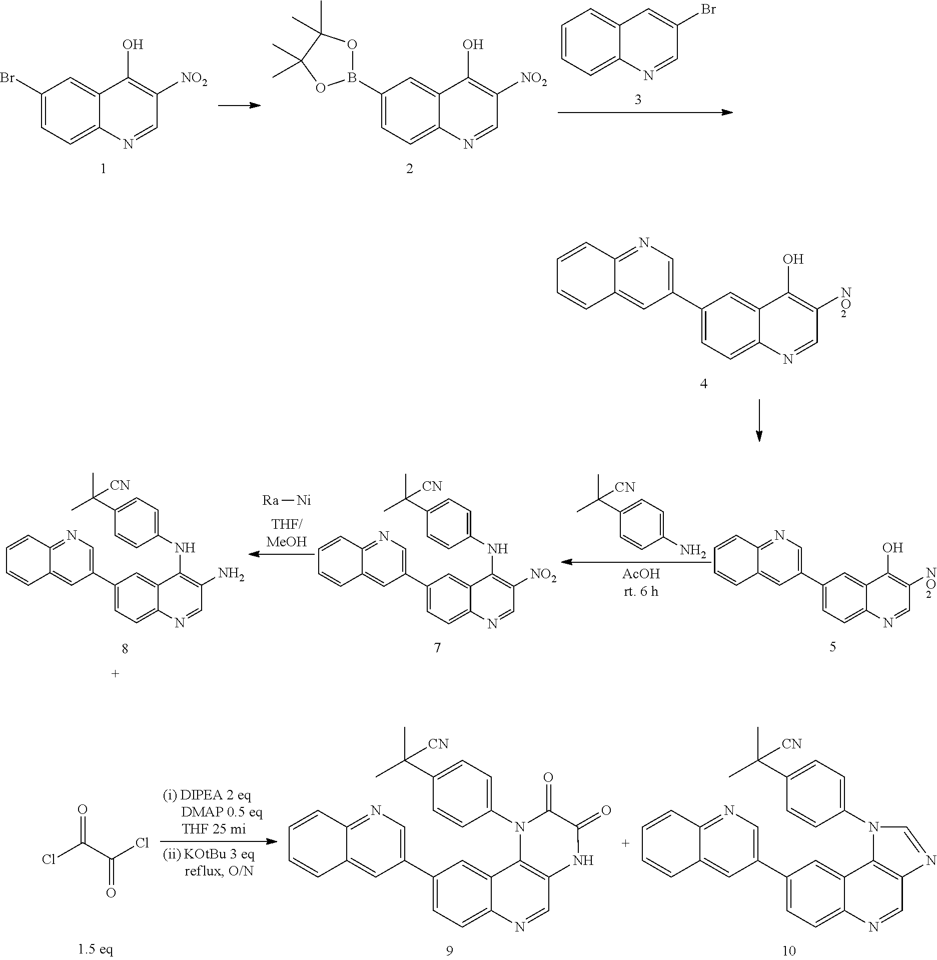

Compound 2 was prepared using bis(pinacolato) diboron, compound 1 in DMSO and PdCl₂(PPh₃)₂ as a catalyst. Then coupled with 3-bromo quinoline 3 by Suzuki coupling method gave compound 4 with 65% yield. After well drying the compound 4 was treated with POCl₃ under nitrogen atmosphere in acetonitrile at 80° C. gives compound 5 in good yield. Then compound 5 was treated with amine 6 in acetic acid at room temperature to product 7. Then compound 7 was reduced by Ra—Ni under hydrogen pressure gave 8. Then compound 8 was treated with oxalyl chloride using DIPEA, DMAP and KOtBu condition gave 2-(4-(2-(methoxymethyl)-8-(quinolin-3-yl)-1H-imidazo[4,5-c]quinolin-1-yl)phenyl)-2-methyl propanenitrile (9, Example 8) and 10.

### Compound 2

Compound 1 (3 g, 0.011 mol, 1 eq) was dissolved in 15 ml of DMSO and bis(pinacolato)diboron (3.07 g, 0.012 mol, 1.1 eq), KOAc (3.2 g, 0.032 mol, 3 eq) and PdC1₂(PPh₃)₂ (230 mg, 0.327 mmol, 0.03 eq), was added then reaction mixture was thoroughly degassed by alternately connected the flask to vacuum and nitrogen. This resulting mixture was then heated at 80° C. for overnight. Reaction mixture cooled to room temperature and cooled water (100 ml) was added neutralized to ₚH 7 with 1.5 N HCl. Solid was filtered and washed with water then dried with toluene. The residue is stirred in hexane. The compound 2 was directly taken for next step (4 g, 87%) of off white solid; 1H NMR (DMSO-d6, 300 MHz): [symbol] 13.02s1 [symbol] 9.19 (s, 1H), 8.58 (s, 1H), 7.99 (d, 1H, J=8.40 Hz), 7.70 (d, 1H, J=8.10 Hz), 1.31 (s, 12H); MS (m/z) (M⁺+H): 317 at 1.57 min RT.

### Compound 4

Compound 2 (4 g, 0.012 mol, 1 eq) was dissolved in 20 ml of DME and 3-bromo quinoline (2.6 g, 0.012 mol, 1 eq), Na₂CO₃ (3.2 g, 0.045 mol, 3 eq) and PdCl₂(PPh₃)₂ (260 mg, 0.00037 mol, 0.03 eq) was added then reaction mixture was thoroughly degassed by alternately connected the flask to vacuum and nitrogen. This resulting mixture was then heated at 80° C. for overnight. Reaction mixture cooled to room temperature and cooled water (50 ml) was added neutralized [PH 7] with 1.5 N HCl, then precipitate was formed. Solid was filtered and washed with water then dried with toluene. The residue is stirred in hexane and washed with dichloromethane. The compound 4 was directly taken for next step (2.7 g, 65%) of pale green solid; 1H NMR (DMSO-d6, 300 MHz): [symbol] 9.31 (d, 1H, J=2.25 Hz), 9.16 (s, 1H), 8.76 (s, 1H), 8.65 (d, 1H, J=1.95 Hz), 8.27 (d, 1H, J=2.10 Hz), 8.24 (d, 1H, J=2.13 Hz), 8.13-8.05 (m, 2H), 7.78-7.68 (m, 2H), 7.63-7.56 (m, 1H); MS (m/z) (M⁺+H): 318 at 1.16 min RT

### Compound 5

Compound 4 (500 mg, 1.577 mmol, 1 eq) was dissolved in 5 ml of acetonitrile and DIPEA (447 mg, 3.47 mmol, 2.2 eq) was added then reaction mixture cooled to 0° C. and POCl₃ (482 mg, 3.154 mmol, 2 eq) was added drop wise then slowly raised to 80° C. for 2 hours. After reaction completion (monitored by TLC), The reaction mixture is cooled to room temperature and removed the solvent under vacuum. Then the crude was poured into ice-water (10 ml). Neutralized with 10% aq NaHCO₃ and was extracted with dichloromethane (4×25 ml) and organic layer was dried over Na₂S0₄, the solvent is evaporated to dryness. The compound 5 was purified by flash column (silica gel, 8:2 [hexane:ethyl acetate]) to provide off white solid (480 mg, 84%); 1H NMR (DMSO-d6, 300 MHz): □9.42 (s, 1H), 9.32 (s, 1H), 8.94 (s, 1H), 8.80 (s, 1H), 8.62 (d, 1H, J=8.97 Hz), 8.38 (d, 1H, J=8.88 Hz), 8.15-8.12 (m, 2H), 7.86-7.80 (m, 1H), 7.69-7.61 (m, 1H); MS (m/z) (M⁺+H): 335 at 3.29 min RT.

### Compound 7

Compound 5 (2.5 g, 7.46 mmol, 1 eq) was dissolved in 25 ml of AcOH and amine 6 (1.1 g, 7.46 mmol, 1 eq) was added then reaction mixture was stirred for 4 hours at room temperature under Nitrogen. The reaction mixture poured into water (50 ml) and extracted with dichloromethane (5×100 ml), dried over Na₂SO₄. Yield: 2 g. (58%) of yellow solid; ¹H NMR (DMSO-d₆, 400 MHz): □ 10.25 (s, 1H), 9.24 (s, 1H), 9.12 (s, 1H), 8.87 (s, 1H), 8.43 (d, 1H, J=11.5 Hz), 8.17 (d, 2H, J=12.24 Hz), 8.08 (d, 1H, J=11.9 Hz), 7.80-7.98 (m, 2H), 7.69-7.67 (m, 1H), 7.53 (d, 2H, J=11.2 Hz), 7.23 (d, 2H, J=11.1 Hz), 1.68 (s, 6H); MS (m/z) (M⁺+H): 460 at 3.40 min RT in positive mode.

### Compound 8

Compound 7 (250 mg, 0.544 mmol, 1 eq) was dissolved in 10 ml of MeOH/THF (1:1) and Ra—Ni (150 mg) was added then reaction mixture was stirred for 3 hours under 30 psi hydrogen pressure. Reaction mixture filtered and solvent was removed under vacuum. Purified by column over basic silica gel [CHCl₃:MeOH 9:1] Yield: 110 mg. (47%) of pale yellow solid; ¹H NMR (DMSO-d₆, 300 MHz): □ 9.23 (s, 1H), 8.62 (s, 3H), 8.16 (s, 1H), 8.17 (d, 2H, J=8.2 Hz), 8.08 (d, 1H, J=8.9 Hz), 8.05-8.00 (m, 3H), 7.73-7.71 (m, 1H), 7.52-7.50 (m, 1H), 7.29 (d, 2H, J=8.3 Hz), 6.64 (d, 2H, J=8.7 Hz), 1.60 (s, 6H); MS (m/z) (M⁺+H): 430 at 4.24 min RT in positive mode.

### Compound 9. 2-(4-(2,3-dioxo-9-(quinolin-3-yl)-3,4-dihydropyrazino[2,3-c]quinolin-1(2H)-yl)phenyl)-2-methylpropanenitrile (1026)

Compound 8 (200 mg, 0.466 mmol, 1 eq) in 10 ml of THF, DIPEA (120 mg, 0.932 mmol, 2 eq) and DMAP (28 mg, 0.233 mmol, 0.5 eq) were added and stirred for 30 min at room temperature. Reaction mixture was cooled to 0° C. Oxalylchloride (118 mg, 0.932 mmol, 2 eq) in 10 ml of THF was added drop wise then reaction mixture was stirred for 3 hours. Then KOtBu (160 mg, 1.39 mmol, 3 eq) was added refluxed for overnight. Reaction mixture was extracted with dichloromethane (4×50 ml). Combined organic layer washed with brine dried over Na₂SO₄. The compound 9 and 10 was purified by preparative HPLC. Yield: 7 mg. (3% for 9) off white solid; 9 mg. (4% for 10) off white solid.

For 9 ¹H NMR (DMSO-d₆, 400 MHz): □ 12.65 (s, 1H), 8.87 (s, 1H), 8.40 (d, 2H, J=11.16 Hz), 8.14 (d, 1H, J=8.68 Hz), 8.04-7.99 (m, 3H), 7.82-7.78 (m, 31-1), 7.67-7.63 (m, 3H), 7.13 (s, 1H), 1.64 (s, 6H); MS (m/z) (M⁺+H): 484 at 3.49 min RT in positive mode.

| Cpd | | | Mol. | | MS | |
|---|---|---|---|---|---|---|
| No | Ex | Structure | Formula | MW | (M⁺ + H) | IUPAC |
| 1337 | 1 | 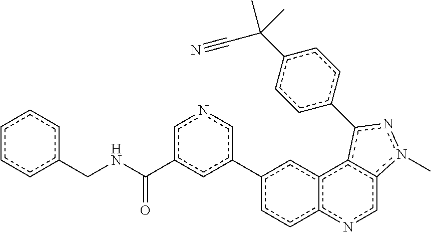 | C34H28N6O | 536.6 | 537 | N-benzyl-5-(1-(4-(2- cyanopropan-2-yl)phenyl)-3- methyl-3H-pyrazolo[3,4- c]quinolin-8-yl)nicotinamide |
| 1338 | 2 | 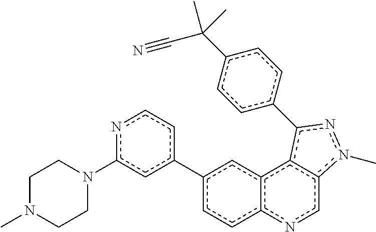 | C31H31N7 | 501.6 | 502 | 2-methyl-2-(4-(3-methyl-8-(2- (4-methylpiperazin-1- yl)pyridin-4-yl)-3H- pyrazolo[3,4-c]quinolin-1- yl)phenyl)propanenitrile |
| 1339 | 3 | 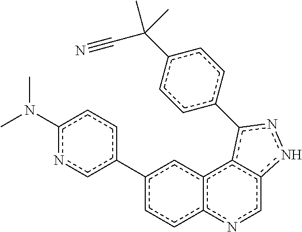 | C27H24N6 | 432.5 | 433 | 2-(4-(8-(6- (dimethylamino)pyridin-3-yl)- 3H-pyrazolo[3,4-c]quinolin-1- yl)phenyl)-2- methylpropanenitrile |
| 1340 | 4 | 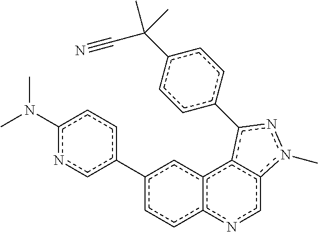 | C28H26N6 | 446.5 | 447 | 2-(4-(8-(6- (dimethylamino)pyridin-3-yl)- 3-methyl-3H-pyrazolo[3,4- c]quinolin-1-yl)phenyl)-2- methylpropanenitrile |
| 1341 | 5 | 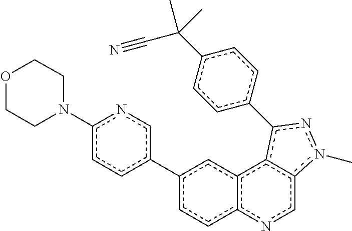 | C30H28N6O | 488.6 | 489 | 2-methyl-2-(4-(3-methyl-8-(6- morpholinopyridin-3-yl)-3H- pyrazolo[3,4-c]quinolin-1- yl)phenyl)propanenitrile |
| 1342 | 6 | 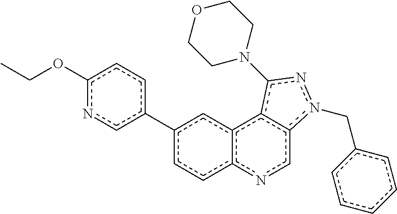 | C28H27N5O2 | 465.5 | 466 | 3-benzyl-8-(6-ethoxypyridin- 3-yl)-1-morpholino-3H- pyrazolo[3,4-c]quinoline |
| 1343 | 7 | 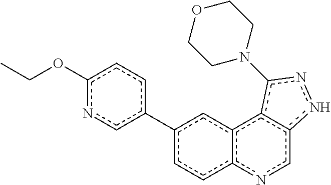 | C21H21N5O2 | 375.4 | 376 | 8-(6-ethoxypyridin-3-yl)-1- morpholino-3H-pyrazolo[3,4- c]quinoline |
| 1344 | 8 | 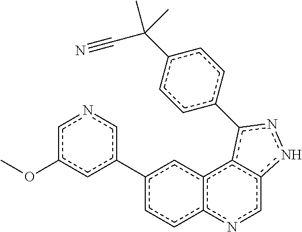 | C26H21N5O | 419.5 | 420 | 2-(4-(8-(5-methoxypyridin-3- yl)-3H-pyrazolo[3,4- c]quinolin-1-yl)phenyl)-2- methylpropanenitrile |
| 1345 | 9 | 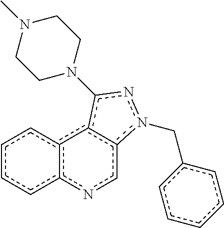 | C22H23N5 | 357.5 | 358 | 3-benzyl-1-(4- methylpiperazin-1-yl)-3H- pyrazolo[3,4-c]quinoline |
| 1346 | 10 | 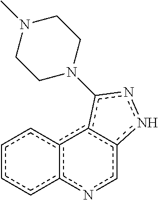 | C15H17N5 | 267.3 | 268 | 1-(4-methylpiperazin-1-yl)- 3H-pyrazolo[3,4-c]quinoline |
| 1347 | 11 | 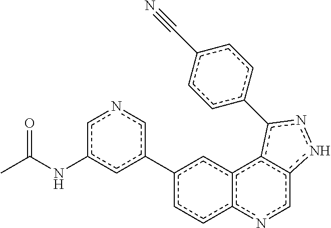 | C24H16N6O | 404.4 | 405 | N-(5-(1-(4-cyanophenyl)-3H- pyrazolo[3,4-c]quinolin-8- yl)pyridin-3-yl)acetamide |
| 1348 | 12 | 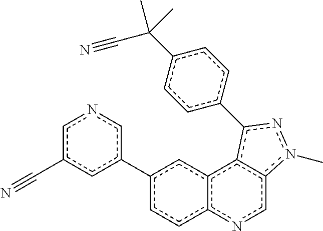 | C27H20N6 | 428.5 | 447 | 5-(1-(4-(2-cyanopropan-2- yl)phenyl)-3-methyl-3H- pyrazolo[3,4-c]quinolin-8- yl)picolinamide |
| 1349 | 13 | 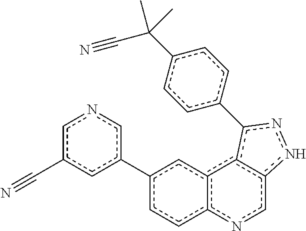 | C26H18N6 | 414.5 | 487 | N-(5-(1-(4-(2-cyanopropan- 2-yl)phenyl)-2-methyl-2H- pyrazolo[3,4-c]quinolin-8- yl)pyridin-3-yl) cyclopropanecarboxamide |
| 1350 | 14 | 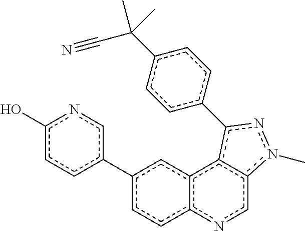 | C26H21N5O | 419.5 | 488 | 2-methyl-2-(4-(2-methyl-8- (5-morpholinopyridin- 3-yl)-2H-pyrazolo[3,4-c] quinolin-1-yl)phenyl) propanenitrile |
| 1351 | 15 | 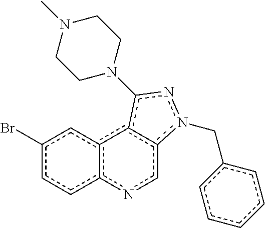 | C22H22BrN5 | 436.3 | 461 | N-(4-(1-(4-(2-cyanopropan- 2-yl)phenyl)-3-methyl-3H- pyrazolo[3,4-c]quinolin-8- yl)pyridin-2-yl)acetamide |
| 1352 | 16 | 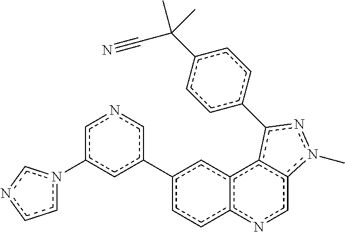 | C29H23N7 | 469.5 | 471 | 2-methyl-2-(4-(3-methyl-8- (6-(oxazol-2-yl)pyridin- 3-yl)-3H-pyrazolo[3,4-c] quinolin-1- yl)phenyl)propanenitrile |
| 1353 | 17 | 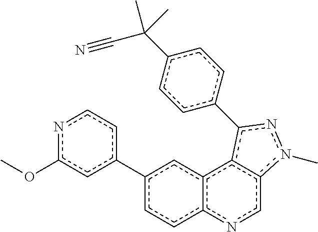 | C27H23N5O | 433.5 | 470 | 2-(4-(8-(6-(1H-pyrazol-1- yl)pyridin-3-yl)-3-methyl- 3H-pyrazolo[3,4-c] quinolin-1-yl)phenyl)-2- methylpropanenitrile |
| 1354 | 22 | 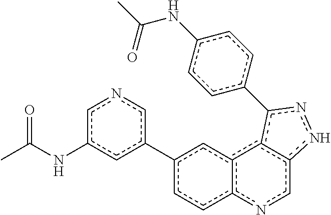 | C25H20N6O2 | 436.5 | 537 | N-(5-(3-benzyl-1-(4-(2- cyanopropan-2-yl)phenyl)- 3H-pyrazolo[3,4-c] quinolin-8-yl)pyridin-3- yl)acetamide |
| 1355 | 23 | 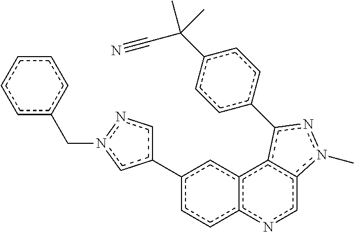 | C31H26N6 | 482.6 | 573 | N-(5-(3-benzyl-1-(4-(2- cyanopropan-2-yl)phenyl)- 3H-pyrazolo[3,4-c] quinolin-8-yl)pyridin-3- yl)methanesulfonamide |
| 1356 | 24 | 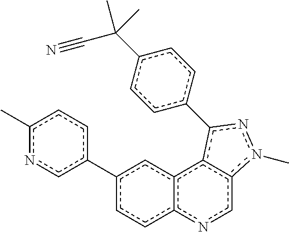 | C27H23N | 417.5 | 537 | N-(5-(2-benzyl-1-(4-(2- cyanopropan-2-yl)phenyl)- 2H-pyrazolo[3,4-c] quinolin-8-yl)pyridin- 3-yl)acetamide |
| 1357 | 25 | 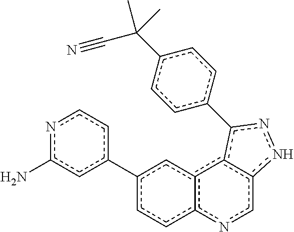 | C25H20N6 | 404.5 | 573 | N-(5-(2-benzyl-1-(4-(2- cyanopropan-2-yl)phenyl)- 2H-pyrazolo[3,4-c]quinolin- 8-yl)pyridin-3- yl)methanesulfonamide |
| 1358 | 26 | 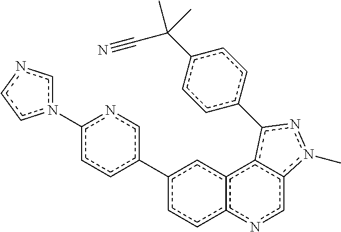 | C29H23N7 | 469.5 | 429 | 5-(1-(4-(2-cyanopropan-2- yl)phenyl)-3-methyl-3H- pyrazolo[3,4-c]quinolin-8- yl)nicotinonitrile |
| 1359 | 27 | 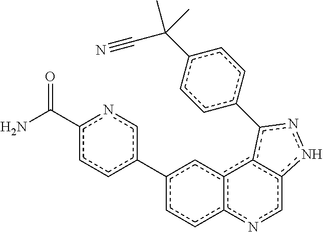 | C26H20N6O | 432.5 | 415; M + Na 437 | 5-(1-(4-(2-cyanopropan-2- yl)phenyl)-3H-pyrazolo [3,4-c]quinolin-8-yl) nicotinonitrile |
| 1360 | 28 | 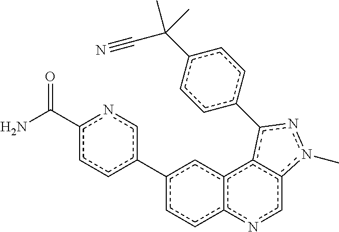 | C27H22N6O | 446.5 | 420 | 2-(4-(8-(6-hydroxypyridin- 3-yl)-3-methyl-3H- pyrazolo[3,4-c]quinolin-1- yl)phenyl)-2- methylpropanenitrile |
| 1361 | 29 | 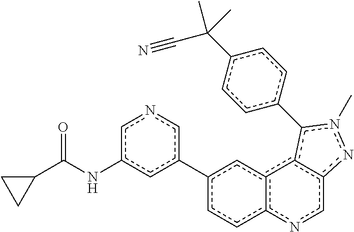 | C30H26N6O | 486.6 | 436, 438 | 3-benzyl-8-bromo-1-(4- methylpiperazin-1-yl)-3H- pyrazolo[3,4-c]quinoline |
| 1362 | 30 | 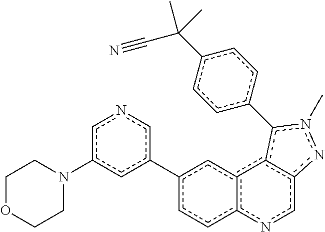 | C30H28N6O | 488.6 | 470 | 2-(4-(8-(5-(1H-imidazol- 1-yl)pyridin-3-yl)-3- methyl-3H-pyrazolo[3,4-c] quinolin-1-yl)phenyl)-2- methylpropanenitrile |
| 1363 | 31 | 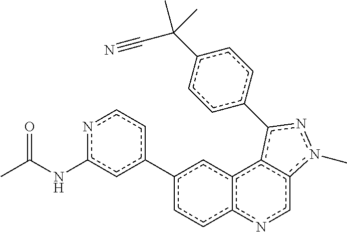 | C28H24N6O | 460.5 | 433 | 2-(4-(8-(2-methoxypyridin- 4-yl)-3-methyl-3H- pyrazolo[3,4-c]quinolin-1- yl)phenyl)-2- methylpropanenitrile |
| 1364 | 32 | 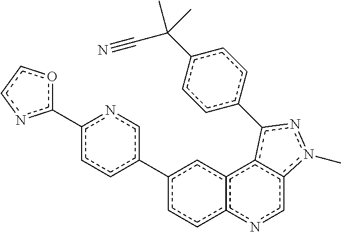 | C29H22N6O | 470.5 | 437 | N-(5-(1-(4- acetamidophenyl)-3H- pyrazolo[3,4-c]quinolin-8- yl)pyridin-3-yl)acetamide |
| 1365 | 33 | 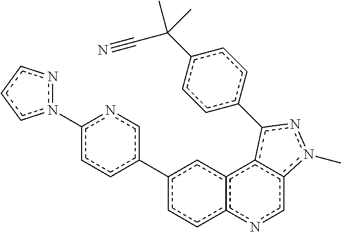 | C29H23N7 | 469.5 | 586 | 1-(2-benzyl-2H-indazol-4- yl)-6-((4-(methylsulfonyl) piperazin-1-yl)methyl)-3- morpholino-1H-indazole |
| 1366 | 34 | 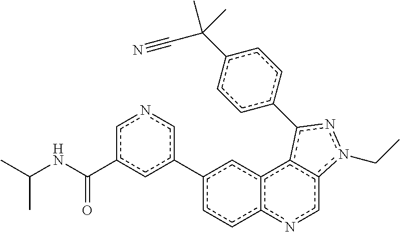 | C31H30N6O | 502.6 | 418 | 2-methyl-2-(4-(3-methyl-8- (6-methylpyridin-3-yl)-3H- pyrazolo[3,4-c]quinolin-1- yl)phenyl)propanenitrile |
| 1367 | 35 | 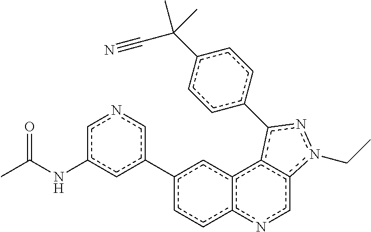 | C29H26N6O | 474.6 | 404 | 2-(4-(8-(2-aminopyridin-4- yl)-3H-pyrazolo[3,4-c] quinolin-1-yl)phenyl)-2- methylpropanenitrile |
| 1368 | 36 | 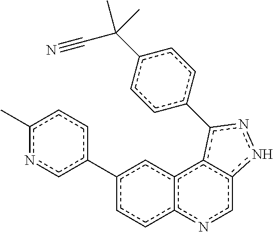 | C26H21N5 | 403.5 | 470 | 2-(4-(8-(6-(1H-imidazol-1- yl)pyridin-3-yl)-3-methyl-3H- pyrazolo[3,4-c]quinolin-1- yl)phenyl)-2- methylpropanenitrile |
| 1369 | 37 | 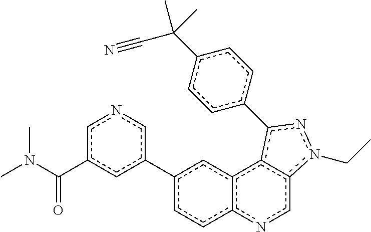 | C30H28N6O | 488.6 | 433 | 5-(1-(4-(2-cyanopropan-2- yl)phenyl)-3H-pyrazolo[3,4- c]quinolin-8-yl)picolinamide |
| 1370 | 38 | 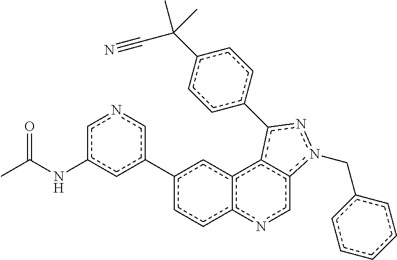 | C34H28N6O | 536.6 | 496 | 1-(1H-indazol-4-yl)-6-((4- (methylsulfonyl)piperazin- 1-yl)methyl)-3-morpholino- 1H-indazole |
| 1371 | 39 | 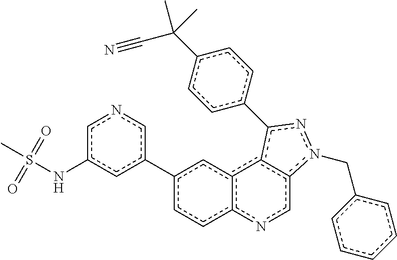 | C33H28N6O2S | 572.7 | 586 | 1-(2-benzyl-2H-indazol-4- yl)-5-((4-(methylsulfonyl) piperazin-1-yl)methyl)- 3-morpholino-1H-indazole |
| 1372 | 40 | 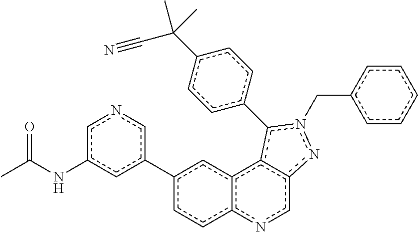 | C34H28N6O | 536.6 | 496 | 1-(1H-indazol-4-yl)-5-((4- (methylsulfonyl)piperazin-1- yl)methyl)-3-morpholino- 1H-indazole |
| 1373 | 41 | 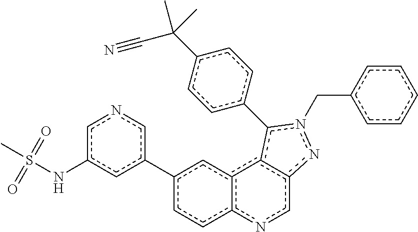 | C33H28N6O2S | 572.7 | 483 | 2-(4-(8-(1-benzyl-1H- pyrazol-4-yl)-3-methyl- 3H-pyrazolo[3,4-c] quinolin-1 yl)phenyl)-2- methylpropanenitrile |

While the invention has been particularly shown and described with reference to particular embodiments, it will be appreciated that variations of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. A novel quinoline compound according to Formula(I) 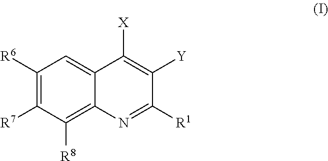
or stereoisomers, prodrugs, or pharmaceutically acceptable salt forms thereof, wherein:
X is NR² or CR², forming a 5 or 6 membered quinoline-fused heterocycle;
Y is NR³, CR³, or O, forming a 5 or 6 membered quinoline-fused heterocycle;
with the proviso that in said 5-membered quinoline fused heterocyle X cannot be NR²;
R¹ is H, OH, or O(C₁-C8)R^{1a},
C₁-C₈ alkyl substituted with 0-3 R^{1a},
C₂-C₈ alkenyl substituted with 0-3 R^{1a},
C₂-C₈ alkynyl substituted with 0-3 R^{1a},
C₂-C₈ alkoxy substituted with 0-3 R^{1a},
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, or
5 to 10 member heterocycle containing 1 to 4 heteroatoms selected from nitrogen, oxygen, and sulfur, wherein said 5 to 10 member heterocycle is substituted with 0-3 R^{1b};
R^{1a}, at each occurrence, is independently selected from is H, Cl, F, Br, I, CN, NO₂, NR¹²R¹³, OR⁵, SR⁴, C(═O)R₄, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, and
5 to 10 member heterocycle containing 1 to 4 heteroatoms selected from nitrogen, oxygen, and sulphur, wherein said 5 to 10 membered heterocycle is substituted with 0-3 R^{1b};
with the proviso that said heterocycle is not imidazo
R^{1b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
with the proviso that R¹ is not 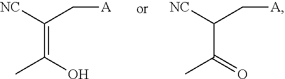
where A is B—(CH₂)ₙ—R^{1c},
B is —CONH—, —SO₂— or —CO—,
n is 1-6, and
R^{1c} is C₁-C₁₄ alkyl,
phenyl,
unsaturated 5-member heterocycle containing 2 or 3 heteroatoms selected from nitrogen, oxygen, and sulfur,
wherein the phenyl and the unsaturated 5-member heterocycle are substituted with
0-2 substituents selected independently from halogen, CF₃, hydroxyl, nitro, amino, formylamino, C₁-C₆ alkyl, C₁-C₆ alkoxy, C₂-C₈ alkanoylamino and C₂-C₈ alkanoyloxy;
R² is H,
C₁-C₈ alkyl substituted with 0-3 R^{2a},
C₂-C₈ alkenyl substituted with 0-3 R^{2a},
C₂-C₈ alkynyl substituted with 0-3 R^{2a},
C₃-C₁₀ carbocycle substituted with 0-3 R^{2b},
C₁-C₄ sulfonamido substituted with 0-3 R^{2b},
C₆-C₁₀ aryl substituted with 0-3 R^{2b}, or
5 to 10 member heterocycle containing 1 to 4 heteroatoms selected from nitrogen, oxygen, and sulfur, wherein said 5 to 10 member heterocycle is substituted with 0-3 R^{2b},
R^{2a}, at each occurrence, is independently selected from is H, Cl, F, Br, I, CN, NO₂, NR¹²R¹³, OR₅, SR⁴, C(═O)R₄, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{2b},
C₁-C₄ sulfonamido substituted with 0-3 R^{2b},
aryl, arylamine, or allyloxy, at each occurrence substituted with 0-3 R^{2b}, and 5 to 10 member heterocycle containing 1 to 4 heteroatoms selected from nitrogen, oxygen, and sulphur, wherein said 5 to 10 member heterocycle is substituted with 0-3 R^{2b};
R^{2b}, at each occurrence, is independently selected from H, OH, Cl, F, Br, I, CN, NO₂, thiazole, NR¹₂R¹³, CF₃, acetyl, SCH₃, S(═O)CH₃, S(═O)₂CH₃, H₂N—C(═O)—,
C₁-C₆ alkyl, C₁-C₄ alkoxy, C₁-C₄ haloalkyl, C₁-C₆ cyanoalkyl,
C₁-C₄ haloalkoxy, C₁-C₄ cyanoalkoxy, and C₁-C₄ haloalkyl-S—;
R³ is H, O, or S,
C₁-C8 alkyl substituted with 0-3 R^{3a},
C₂-C₈ alkenyl substituted with 0-3 R^{3a},
C₂-C₈ alkynyl substituted with 0-3 R^{3a},
C₂-C₈ alkoxy substituted with 0-3 R^{3a},
C₃-C₁₀ carbocycle substituted with 0-3 R^{3b},
C₁-C₄ sulfonamido substituted with 0-3 R^{3b},
C₆-C₁₀ aryl substituted with 0-3 R^{3b}, or
5 to 10 member heterocycle containing 1 to 4 heteroatoms selected from nitrogen, oxygen, and sulfur, wherein said 5 to 10 member heterocycle is substituted with 0-3 R^{3b},
R^{3a}, at each occurrence, is independently selected from is H, Cl, F, Br, I, CN, NO₂, NR¹²R¹³, OR⁵, SR⁴, C(═O)R⁴ providing the NR² is not substituted by R^{2a} being C(═O)R⁴,
NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{3b},
C₁-C₄ sulfonamido substituted with 0-3 R^{3b},
C₆-C10 aryl substituted with 0-3 R^{3b}, and
5 to 10 member heterocycle containing 1 to 4 heteroatoms selected from nitrogen, oxygen, and sulphur, wherein said 5 to 10 member heterocycle is substituted with 0-3 R^{3b};
R^{3b}, at each occurrence, is independently selected from H, OH, Cl, F, Br, I, CN, NO₂, NR¹²R¹³, CF₃, acetyl, SCH₃, S(═O)CH₃, S(═O)₂CH₃, H₂N—C(═O)—,
C₁-C₆ alkyl, C₁-C₄ alkoxy, C₁-C₄ haloalkyl, C₁-C₆ cyanoalkyl,
C₁-C₄ haloalkoxy, C₁-C₄ cyanoalkoxy, and C₁-C₄ haloalkyl-S—;
R⁴ is H, phenyl, benzyl, C₁-C₄ alkyl, C₃-C₈ cycloalkyl substituted with 0-3 R^{1b}, or
a 5 to 10 member heterocycle containing 1 to 4 heteroatoms selected from nitrogen, oxygen, and sulphur, wherein said 5 to 10 member heterocycle is substituted with 0-3 R^{1b};
R⁵ is H, phenyl, benzyl, or C₁-C₄ alkyl;
R⁶ is H,
C₁-C₈ alkyl substituted with 0-3 R^{6a},
C₂-C₈ alkenyl substituted with 0-3 R^{6a},
C₂-C₈ alkynyl substituted with 0-3 R^{6a},
C₃-C₁₀ carbocycle substituted with 0-3 R^{6b},
C₁-C₄ sulfonamido substituted with 0-3 R^{6b},
aryl, arylamine, or alkyloxy, at each occurrence substituted with 0-3 R^{6b}, or
5 to 10 member heterocycle containing 1 to 4 heteroatoms selected from nitrogen, oxygen, and sulfur, wherein said 5 to 10 member heterocycle is substituted with 0-3 R^{6b},
except where R⁶ is in the form of —C(R^{6c})(R^{6d})—NH—CH(R^{6e})(R^{6f}),
wherein R^{6c} and R^{6d} are independently H, C₁₋₄ haloalkyl or C₁₋₈ alkyl, and
R^{6e} is a C₁₋₈alkyl or C₁₋₈ alkyl or C₁₋₄ haloalkyl, and
R^{6f} is phenyl, benzyl, naphthyl or saturated or unsaturated 5- or 6-membered heterocycle containing 1, 2 or 3 atoms selected from nitrogen, oxygen and sulphur with no more than two substituent atoms selected from oxygen and sulphur, and
wherein said phenyl, benzyl or heterocycle contain 0-3 substituents selected from C₁₋₆ alkyl, C₁₋₄ haloalkyl, —OC₁₋₆alkyl, halogen, cyano and nitro;
R^{6a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵, C(═O)NH₂,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, and
5 to 10 member heterocycle containing 1 to 4 heteroatoms selected from nitrogen, oxygen, and sulphur, wherein said 5 to 10 member heterocycle is substituted with 0-3 R^{1b};
R^{6b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R⁷ is C₁-C₄ alkyl, C₂-C₄ alkenyl, or C₃-C₄ alkynyl;
R⁸ is H,
C₁-C₈ alkyl substituted with 0-3 R^{8a},
C₂-C₈ alkenyl substituted with 0-3 R^{8a},
C₂-C₈ alkynyl substituted with 0-3 R^{8a}
C₃-C₁₀ carbocycle substituted with 0-3 R^{8b},
C₁-C₄ sulfonamido substituted with 0-3 R^{8b},
C₆-C₁₀ aryl substituted with 0-3 R^{8b}, or
5 to 10 member heterocycle containing 1 to 4 heteroatoms selected from nitrogen, oxygen, and sulfur, wherein said 5 to 10 member heterocycle is substituted with 0-3 R^{8b},
R^{8a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{8b},
C₁-C₄ sulfonamido substituted with 0-3 R^{8b},
C₆-C₁₀ aryl substituted with 0-3 R^{8b}, and
5 to 10 member heterocycle containing 1 to 4 heteroatoms selected from nitrogen, oxygen, and sulphur, wherein said 5 to 10 member heterocycle is substituted with 0-3 R^{8b};
R^{8b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R¹², at each occurrence, is independently selected from H, C₁-C₆ alkyl, benzyl, phenethyl,
(C₁-C₆ alkyl)-C(═O)—, (C₁-C₆ alkyl)-OC(═O)—, (C₁-C₆ alkyl)-S(═O)₂—, and piperdinyl C(═O)—;
R¹³, at each occurrence, is independently selected from H, OH, C₁-C₆ alkyl, benzyl, phenethyl,
(C₁-C₆ alkyl)-C(═O)—, and (C₁-C₆ alkyl)-S(═O)₂—;
alternatively, R¹² and R¹³ together with the nitrogen to which they are attached, may combine to form a 4-7 member ring wherein said 4-7 member ring optionally contains an additional heteroatom selected from O and NH;
R¹⁴, at each occurrence, is independently selected from H, C₁-C₆ alkyl, benzyl, phenethyl, (C₁-C₆ alkyl)-C(═O)—, and (C₁-C₆ alkyl)-S(═O)₂—;
R¹⁵, at each occurrence, is independently selected from H, OH, C₁-C₆ alkyl, benzyl, phenethyl, (C₁-C₆ alkyl)-OC(═O)—,
(C₁-C₆ alkyl)-C(═O)—, and (C₁-C₆ alkyl)-S(═O)₂—; and
alternatively, R¹⁴ and R¹⁵, may combine together with the nitrogen to which they are attached, to form a 4-7 member ring,
wherein said 4-7 member ring optionally contains an heteroatom selected from O and NH.

2. A compound of claim 1, according to Formula(II): 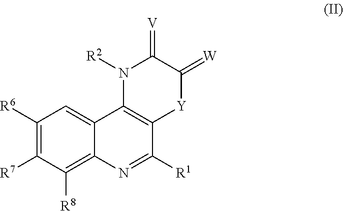
or a stereoisomer or pharmaceutically acceptable salt forms or prodrug thereof,
wherein:
Y is NR³, CR³ or O,
V and W are independently H or O
with the proviso that W is H when V is O; and when W and V are H, Y is not NR³,
R₁ is H, OH,
C₁-C₈ alkyl substituted with 0-3 R^{1a},
C₂-C₈ alkenyl substituted with 0-3 R^{1a},
C₂-C₈ alkynyl substituted with 0-3 R^{1a},
C₂-C₈ alkoxy substituted with 0-3 R^{1a},
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, or
5 to 10 member heterocycle containing 1 to 4 heteroatoms selected from nitrogen, oxygen, and sulfur, wherein said 5 to 10 member heterocycle is substituted with 0-3 R^{1b},
R^{1a}, at each occurrence, is independently selected from is H, Cl, F, Br, I, CN, NO₂, NR¹²R¹³, OR⁵, SR⁴, C(═O)R₄,NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, and
5 to 10 member heterocycle containing 1 to 4 heteroatoms selected from nitrogen, oxygen, and sulphur, wherein said 5 to 10 member heterocycle is substituted with 0-3 R^{1b};
R^{1b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R² is H,
C₁-C₈ alkyl substituted with 0-3 R^{2a},
C₂-C₈ alkenyl substituted with 0-3 R^{2a},
C₂-C₈ alkynyl substituted with 0-3 R^{2a},
C₃-C₁₀ carbocycle substituted with 0-3 R^{2b},
C₁-C₄ sulfonamido substituted with 0-3 R^{2b},
C₆-C₁₀ aryl substituted with 0-3 R^{2b}, or
5 to 10 member heterocycle containing 1 to 4 heteroatoms selected from nitrogen, oxygen, and sulfur, wherein said 5 to 10 member heterocycle is substituted with 0-3 R^{2b},
R^{2a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{2b},
C₁-C₄ sulfonamido substituted with 0-3 R^{2b},
C₆-C₁₀ aryl substituted with 0-3 R^{2b}, and
5 to 10 member heterocycle containing 1 to 4 heteroatoms selected from nitrogen, oxygen, and sulphur, wherein said 5 to 10 member heterocycle is substituted with 0-3 R^{2b};
R^{2b}, at each occurrence, is independently selected from H, OH, Cl, F, Br, I, CN, NO₂, thiazole, NR¹²R¹³, CF₃, acetyl, SCH₃, S(═O)CH₃, S(═O)₂CH₃, H₂N—C(═O)—,
C₁-C₆ alkyl, C₁-C₄ alkoxy, C₁-C₄ haloalkyl, C₁-C₆ cyanoalkyl,
C₁-C₄ haloalkoxy, C₁-C₄ cyanoalkoxy, and C₁-C₄ haloalkyl-S—;
R³ is H,
C₁-C₈ alkyl substituted with 0-3 R^{3a},
C₂-C₈ alkenyl substituted with 0-3 R^{3a},
C₂-C₈ alkynyl substituted with 0-3 R^{3a}
C₃-C₁₀ carbocycle substituted with 0-3 R^{3b},
C₁-C₄ sulfonamido substituted with 0-3 R^{3b},
C₆-C₁₀ aryl substituted with 0-3 R^{3b}, or
5 to 10 member heterocycle containing 1 to 4 heteroatoms selected from nitrogen, oxygen, and sulfur, wherein said 5 to 10 member heterocycle is substituted with 0-3 R^{3b},
R^{3a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{3b},
C₁-C₄ sulfonamido substituted with 0-3 R^{3b},
C₆-C₁₀ aryl substituted with 0-3 R^{3b}, and
5 to 10 member heterocycle containing 1 to 4 heteroatoms selected from nitrogen, oxygen, and sulphur, wherein said 5 to 10 member heterocycle is substituted with 0-3 R^{3b};
R^{3b}, at each occurrence, is independently selected from H, OH, Cl, F, Br, I, CN, NO₂, NR¹²R¹³, CF₃, acetyl, SCH₃, S(═O)CH₃, S(═O)₂CH₃, H₂N—C(═O)—,
C₁-C₆ alkyl, C₁-C₄ alkoxy, C₁-C₄ haloalkyl, C₁-C₆ cyanoalkyl,
C₁-C₄ haloalkoxy, C₁-C₄ cyanoalkoxy, and C₁-C₄ haloalkyl-S—;
R⁴ is H, phenyl, benzyl, C₁-C₄ alkyl, C₃-C₈ cycloalkyl substituted with 0-3 R^{1b}, or
a 5 to 10 member heterocycle containing 1 to 4 heteroatoms selected from nitrogen, oxygen, and sulphur, wherein said 5 to 10 member heterocycle is substituted with 0-3 R^{1b};
R⁵ is H, phenyl, benzyl, or C₁-C₄ alkyl;
R⁶ is H,
C₁-C₈ alkyl substituted with 0-3 R^{6a},
C₂-C₈ alkenyl substituted with 0-3 R^{6a},
C₂-C₈ alkynyl substituted with 0-3 R^{6a},
C₃-C₁₀ carbocycle substituted with 0-3 R^{6b},
C₁-C₄ sulfonamido substituted with 0-3 R^{6b},
C₁-C₁₀aryl substituted with 0-3 R^{6a}; arylamine substituted with 0-3 R^{6a},
C₁-C₆ alkyloxy substituted with 0-3 R^{6a}, or
5 to 10 member heterocycle containing 1 to 4 heteroatoms selected from nitrogen, oxygen, and sulfur, wherein said 5 to 10 member heterocycle is substituted with 0-3 R^{6b},
R^{6a}, at each occurrence, is independently selected from is H, Cl, F, Br, I, CN, NO₂, NR¹²R¹³, OR⁵, SR⁴, C(═O)R⁴, NR⁵R⁶, S(═O)R⁶, S(═O)₂R⁶,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, and
5 to 10 member heterocycle containing 1 to 4 heteroatoms selected from nitrogen, oxygen, and sulphur, wherein said 5 to 10 member heterocycle is substituted with 0-3 R^{1b};
R^{6b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R⁷ is C₁-C₄ alkyl, C₂-C₄ alkenyl, or C₃-C₄ alkynyl;
R⁸ is H,
C₁-C₈ alkyl substituted with 0-3 R^{8a},
C₂-C₈ alkenyl substituted with 0-3 R^{8a},
C₂-C₈ alkynyl substituted with 0-3 R^{8a}
C₃-C₁₀ carbocycle substituted with 0-3 R^{8b},
C₁-C₄ sulfonamido substituted with 0-3 R^{8b},
C₆-C₁₀ aryl substituted with 0-3 R^{8b}, or
5 to 10 member heterocycle containing 1 to 4 heteroatoms selected from nitrogen, oxygen, and sulfur, wherein said 5 to 10 member heterocycle is substituted with 0-3 R^{8b},
R^{8a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R⁴,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{8b},
C₁-C₄ sulfonamido substituted with 0-3 R^{8b},
C₆-C₁₀ aryl substituted with 0-3 R^{8b}, and
5 to 10 member heterocycle containing 1 to 4 heteroatoms selected from nitrogen, oxygen, and sulphur, wherein said 5 to 10 member heterocycle is substituted with 0-3 R^{8b};
R^{8b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R¹², at each occurrence, is independently selected from H, C₁-C₆ alkyl, benzyl, phenethyl, (C₁-C₆ alkyl)—C═O)—, (C₁-C₆ alkyl)—OC(═O)—, (C₁-C₆ alkyl)-S(═O)₂—, and piperdinyl C(═O)—;
R¹³, at each occurrence, is independently selected from H, OH, C₁-C₆ alkyl, benzyl, phenethyl,
(C₁-C₆ alkyl)-C(═O)—, and (C₁-C₆ alkyl)-S(═O)₂—;
alternatively, R¹² and R¹³ together with the nitrogen to which they are attached, may combine to form a 4-7 member ring wherein said 4-7 member ring optionally contains an additional heteroatom selected from O and NH;
R¹⁴, at each occurrence, is independently selected from H, C₁-C₆ alkyl, benzyl, phenethyl, (C₁-C₆ alkyl)-C(═O)—, and (C₁-C₆ alkyl)-S(═O)₂—;
R¹⁵, at each occurrence, is independently selected from H, OH, C₁-C₆ alkyl, benzyl, phenethyl, (C₁-C₆ alkyl)-OC(═O)—, (C₁-C₆ alkyl)-C(═O)—, and (C₁-C₆ alkyl)-S(═O)₂—; and
alternatively, R¹⁴ and R¹⁵, may combine together with the nitrogen to which they are attached, to form a 4-7 member ring,
wherein said 4-7 member ring optionally contains an heteroatom selected from O and NH.

3. A compound of claim 1, according to Formula (III), 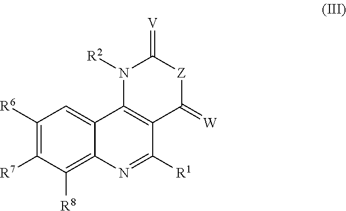
or a stereoisomer or pharmaceutically acceptable salt forms or prodrug thereof,
wherein:
X is N or C;
V and W are independently a single H or O,
W is a single H when V is O;
Z is O, CR³ or NR³;
R¹ is H, O,
C₁-C₈ alkyl substituted with 0-3 R^{1a},
C₂-C₈ alkenyl substituted with 0-3 R^{1a},
C₂-C₈ alkynyl substituted with 0-3 R^{1a},
C₂-C₈ alkoxy substituted with 0-3 R^{1a},
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, or
5 to 10 member heterocycle containing 1 to 4 heteroatoms selected from nitrogen, oxygen, and sulfur, wherein said 5 to 10 member heterocycle is substituted with 0-3 R^{1b},
R^{1a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, and
5 to 10 member heterocycle containing 1 to 4 heteroatoms selected from nitrogen, oxygen, and sulphur, wherein said 5 to 10 member heterocycle is substituted with 0-3 R^{1b};
R^{1b}, at each occurrence, is independently selected from H, OH, Cl, F, Br, I, CN, NO₂, NR¹²R¹³, CF₃, acetyl, SCH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R² is H,
C₁-C₈ alkyl substituted with 0-3 R^{2a},
C₂-C₈ alkenyl substituted with 0-3 R^{2a},
C₂-C₈ alkynyl substituted with 0-3 R^{2a}
C₃-C₁₀ carbocycle substituted with 0-3 R^{2b},
C₁-C₄ sulfonamido substituted with 0-3 R^{2b},
C₆-C₁₀ aryl substituted with 0-3 R^{2b}, or
5 to 10 member heterocycle containing 1 to 4 heteroatoms selected from nitrogen, oxygen, and sulfur, wherein said 5 to 10 member heterocycle is substituted with 0-3 R^{2b},
R^{2a}, at each occurrence, is independently selected from is H, Cl, F, Br, I, CN, NO₂, NR¹²R¹³, OR⁵, SR⁴, C(═O)R⁴, NR14R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{2b},
C₁-C₄ sulfonamido substituted with 0-3 R^{2b},
C₆-C₁₀ aryl substituted with 0-3 R^{2b}, and
5 to 10 member heterocycle containing 1 to 4 heteroatoms selected from nitrogen, oxygen, and sulphur, wherein said 5 to 10 member heterocycle is substituted with 0-3 R^{2b};
R^{2b}, at each occurrence, is independently selected from H, OH, Cl, F, Br, I, CN, NO₂, thiazole, NR¹²R¹³, CF₃, acetyl, SCH₃, S(═O)CH₃, S(═O)₂CH₃, H₂N—C(═O)—,
C₁-C₆ alkyl, C₁-C₄ alkoxy, C₁-C₄ haloalkyl, C₁-C₆ cyanoalkyl,
C₁-C₄ haloalkoxy, C₁-C₄ cyanoalkoxy, and C₁-C₄ haloalkyl-S—;
R³ is H, O,
C₁-C₈ alkyl substituted with 0-3 R3a,
C₂-C₈ alkenyl substituted with 0-3 R^{3a},
C₂-C₈ alkynyl substituted with 0-3 R^{3a}
C₃-C₁₀ carbocycle substituted with 0-3 R^{3b},
C₁-C₄ sulfonamido substituted with 0-3 R³_{b},
C₆-C₁₀ aryl substituted with 0-3 R^{3b}, or
5 to 10 member heterocycle containing 1 to 4 heteroatoms selected from nitrogen, oxygen, and sulfur, wherein said 5 to 10 member heterocycle is substituted with 0-3 R^{3b},
R^{3a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{3b},
C₁-C₄ sulfonamido substituted with 0-3 R^{3b},
C₆-C₁₀ aryl substituted with 0-3 R^{3b}, and
5 to 10 member heterocycle containing 1 to 4 heteroatoms selected from nitrogen, oxygen, and sulphur, wherein said 5 to 10 member heterocycle is substituted with 0-3 R^{3b};
R^{3b}, at each occurrence, is independently selected from H, OH, Cl, F, Br, I, CN, NO₂, NR¹²R¹³, CF₃, acetyl, SCH₃, S(═O)CH₃, S(═O)₂CH₃, H₂N—C(═O)—,
C₁-C₆ alkyl, C₁-C₄ alkoxy, C₁-C₄ haloalkyl, C₁-C₆ cyanoalkyl,
C₁-C₄ haloalkoxy, C₁-C₄ cyanoalkoxy, and C₁-C₄ haloalkyl-S—;
R⁴ is H, phenyl, benzyl, C₁-C₄ alkyl, C₃-C₈ cycloalkyl substituted with 0-3 R^{1b}, or
a 5 to 10 member heterocycle containing 1 to 4 heteroatoms selected from nitrogen, oxygen, and sulphur, wherein said 5 to 10 member heterocycle is substituted with 0-3 R^{1b};
R⁵ is H, phenyl, benzyl, or C₁-C₄ alkyl;
R⁶ is H,
C₁-C₈ alkyl substituted with 0-3 R^{6a},
C₂-C₈ alkenyl substituted with 0-3 R^{6a},
C₂-C₈ alkynyl substituted with 0-3 R^{6a}
C₃-C₁₀ carbocycle substituted with 0-3 R^{6b},
C₁-C₄ sulfonamido substituted with 0-3 R^{6b},
C₆-C₁₀aryl substituted with 0-3 R^{6a}; arylamine substituted with 0-3 R^{6a},
C₁-C₆ alkyloxy substituted with 0-3 R^{6a}, or
5 to 10 member heterocycle containing 1 to 4 heteroatoms selected from nitrogen, oxygen, and sulfur, wherein said 5 to 10 member heterocycle is substituted with 0-3 R^{6b},
R^{6a}, at each occurrence, is independently selected from is H, Cl, F, Br, I, CN, NO₂, NR¹²R¹³, OR₅, SR₄, C(═O)R⁴, NR⁵R⁶, S(═O)R⁶, S(═O)₂R⁶,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{1b},
C₁-C₄ sulfonamido substituted with 0-3 R¹_{b},
C₆-C₁₀ aryl substituted with 0-3 R^{1b}, and
5 to 10 member heterocycle containing 1 to 4 heteroatoms selected from nitrogen, oxygen, and sulphur, wherein said 5 to 10 member heterocycle is substituted with 0-3 R^{1b};
R^{6b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R⁷ is C₁-C₄ alkyl, C₂-C₄ alkenyl, or C₃-C₄ alkynyl;
R⁸ is H,
C₁-C₈ alkyl substituted with 0-3 R^{8a},
C₂-C₈ alkenyl substituted with 0-3 R^{8a},
C₂-C₈ alkynyl substituted with 0-3 R^{8a},
C₃-C₁₀ carbocycle substituted with 0-3 R^{8b},
C₁-C₄ sulfonamido substituted with 0-3 R^{8b},
C₆-C₁₀ aryl substituted with 0-3 R^{8b}, or
5 to 10 member heterocycle containing 1 to 4 heteroatoms selected from nitrogen, oxygen, and sulfur, wherein said 5 to 10 member heterocycle is substituted with 0-3 R^{8b},
R^{8a}, at each occurrence, is independently selected from is H, Cl, F, Br, I, CN, NO₂, NR¹²R¹³, OR⁵, SR₄, C(═O)R₄, NR¹⁴R¹⁵, S(═O)R₆, S(═O)₂R⁴,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{8b},
C₁-C₄ sulfonamido substituted with 0-3 R^{8b},
C₆-C₁₀ aryl substituted with 0-3 R^{8b}, and
5 to 10 member heterocycle containing 1 to 4 heteroatoms selected from nitrogen, oxygen, and sulphur, wherein said 5 to 10 member heterocycle is substituted with 0-3 R^{8b};
R^{8b}, at each occurrence, is independently selected from H, OH, Cl, F, Br, I, CN, NO₂, NR¹²R¹³, CF₃, acetyl, SCH₃, S(═O)CH₃, S(═O)2CH₃,
C₁-C₆ alkyl, C₁-C₄ alkoxy, C₁-C₄ haloalkyl,
C₁-C₄ haloalkoxy, and C₁-C₄ haloalkyl-S—;
R¹², at each occurrence, is independently selected from H, C₁-C₆ alkyl, benzyl, phenethyl, (C₁-C₆ alkyl)-C(═O)—, (C₁-C₆ alkyl)—OC(═O)—, (C₁-C₆ alkyl)-S(═O)₂—, and piperdinyl C(═O)—;
R¹³, at each occurrence, is independently selected from H, OH, C₁-C₆ alkyl, benzyl, phenethyl,
(C₁-C₆ alkyl)-C(═O)—, and (C₁-C₆ alkyl)-S(═O)₂—;
alternatively, R¹² and R¹³ together with the nitrogen to which they are attached, may combine to form a 4-7 member ring wherein said 4-7 member ring optionally contains an additional heteroatom selected from O and NH;
R¹⁴, at each occurrence, is independently selected from H, C₁-C₆ alkyl, benzyl, phenethyl, (C₁-C₆ alkyl)-C(═O)—, and (C₁-C₆ alkyl)-S(═O)₂—;
R¹⁵, at each occurrence, is independently selected from H, OH, C₁-C₆ alkyl, benzyl, phenethyl, (C₁-C₆ alkyl)—OC(═O)—,
(C₁-C₆ alkyl)-C(═O)—, and (C₁-C₆ alkyl)-S(═O)₂—; and
alternatively, R¹⁴ and R¹⁵, may combine together with the nitrogen to which they are attached, to form a 4-7 member ring,
wherein said 4-7 member ring optionally contains an heteroatom selected from O and NH.

4. A compound of claim 1, according to Formula (IV), 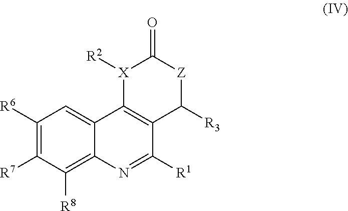
or stereoisomer or pharmaceutically acceptable salt forms or prodrug thereof,
wherein:
Z is O, CR³ or NR³ and all other symbols are as described in III of claim 3.

5. A compound of claim 1, according to Formula (V), 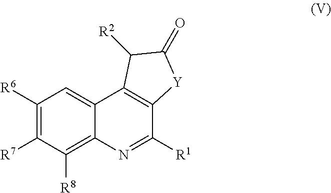
or a stereoisomer or pharmaceutically acceptable salt forms or prodrug thereof,
wherein:
Y is O, CR³ or NR³;
R^{I} is H, O,
C₁-C₈ alkyl substituted with 0-3 R^{1a},
C₂-C₈ alkenyl substituted with 0-3 R^{1a},
C₂-C₈ alkynyl substituted with 0-3 R^{1a},
C₂-C₈ alkoxy substituted with 0-3 R^{1a},
C₃-C₁₀ carbocycle substituted with 0-3 R¹_{b},
C₁-C₄ sulfonamido substituted with 0-3 R^{1b},
C₆-C₁₀ aryl substituted with 0-3 R^{1b}, or
5 to 10 member heterocycle containing 1 to 4 heteroatoms selected from nitrogen, oxygen, and sulfur, wherein said 5 to 10 member heterocycle is substituted with 0-3 R^{1b},
R^{1a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R_{1b},
C₁-C₄ sulfonamido substituted with 0-3 R_{1b},
C₆-C₁₀ aryl substituted with 0-3 R_{1b}, and
5 to 10 member heterocycle containing 1 to 4 heteroatoms selected from nitrogen, oxygen, and sulphur, wherein said 5 to 10 member heterocycle is substituted with 0-3 R_{1b};
R^{1b}, at each occurrence, is independently selected from H, OH, Cl, F, Br, I, CN, NO₂, NR₁₂R₁₃, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R² is H, O,
C₁-C₈ alkyl substituted with 0-3 R^{2a},
C₂-C₈ alkenyl substituted with 0-3 R^{2a},
C₂-C₈ alkynyl substituted with 0-3 R^{2a}
C₃-C₁₀ carbocycle substituted with 0-3 R^{2b},
C₁-C₄ sulfonamido substituted with 0-3 R^{2b},
C₆-C₁₀ aryl substituted with 0-3 R^{2b}, or
5 to 10 member heterocycle containing 1 to 4 heteroatoms selected from nitrogen, oxygen, and sulfur, wherein said 5 to 10 member heterocycle is substituted with 0-3 R^{2b},
R₂ₐ, at each occurrence, is independently selected from H, Cl, F, Br, I, CN, NO₂, NR₁₂R₁₃, OR⁵, SR⁴, C(═O)R⁴, NR¹⁴R¹⁵, S(═O)R₆, S(═O)7R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{2b},
C₁-C₄ sulfonamido substituted with 0-3 R^{2b},
C₆-C₁₀ C₆-C₁₀ aryl substituted with 0-3 R^{2b}, and
5 to 10 member heterocycle containing 1 to 4 heteroatoms selected from nitrogen, oxygen, and sulphur, wherein said 5 to 10 member heterocycle is substituted with 0-3 R^{2b};
R_{2b}, at each occurrence, is independently selected from H, OH, Cl, F, Br, I, CN, NO₂, thiazole, NR₁₂R₁₃, CF₃, acetyl, SCH₃, S(═O)CH₃, S(═O)₂CH₃, H₂N—C(═O)—,
C₁-C₆ alkyl, C₁-C₄ alkoxy, C₁-C₄ haloalkyl, C₁-C₆ cyanoalkyl,
C₁-C₄ haloalkoxy, C₁-C₄ cyanoalkoxy, and C₁-C₄ haloalkyl-S—;
R₃ is H,
C₁-C₈ alkyl substituted with 0-3 R^{3a},
C₂-C₈ alkenyl substituted with 0-3 R^{3a},
C₂-C₈ alkynyl substituted with 0-3 R^{3a},
C₃-C₁₀ carbocycle substituted with 0-3 R^{3a},
C₁-C₄ sulfonamido substituted with 0-3 R^{3a},
aryl substituted with 0-3 R^{3a}, or
5 to 10 member heterocycle containing 1 to 4 heteroatoms selected from nitrogen, oxygen, and sulfur, wherein said 5 to 10 member heterocycle is substituted with 0-3 R^{3a},
R^{3a}, at each occurrence, is independently selected from H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{3b},
C₁-C₄ sulfonamido substituted with 0-3 R^{3b},
C₆-C₁₀ aryl substituted with 0-3 R^{3b}, and
5 to 10 member heterocycle containing 1 to 4 heteroatoms selected from nitrogen, oxygen, and sulphur, wherein said 5 to 10 member heterocycle is substituted with 0-3 R^{3b};
R^{3b}, at each occurrence, is independently selected from H, OH, Cl, F, Br, I, CN, NO₂, NR₁₂R₁₃, CF₃, acetyl, SCH₃, S(═O)CH₃, S(═O)₂CH₃, H2N—C(═O)—,
C₁-C₆ alkyl, C₁-C₄ alkoxy, C₁-C₄ haloalkyl, C₁-C₆ cyanoalkyl,
C₁-C₄ haloalkoxy, C₁-C₄ cyanoalkoxy, and C₁-C₄ haloalkyl-S—;
R⁴ is H, phenyl, benzyl, Cl-C₄ alkyl, C₃-C₈ cycloalkyl substituted with 0-3 R^{1b}, or
a 5 to 10 member heterocycle containing 1 to 4 heteroatoms selected from nitrogen, oxygen, and sulphur, wherein said 5 to 10 member heterocycle is substituted with 0-3 R^{1b};
R⁵ is H, phenyl, benzyl, or C₁-C₄ alkyl;
R⁶ is H,
C₁-C₈ alkyl substituted with 0-3 R^{6a},
C₂-C₈ alkenyl substituted with 0-3 R^{6a},
C₂-C₈ alkynyl substituted with 0-3 R^{6a}
C₃-C₁₀ carbocycle substituted with 0-3 R^{6b},
C₁-C₄ sulfonamido substituted with 0-3 R₆^{b},
C₆-C₁₀ aryl substituted with 0-3 R^{6a}; arylamine substituted with 0-3 R^{6a},
C₁-C₆ alkyloxy substituted with 0-3 R^{6a}, or
5 to 10 membered heterocycle containing 1 to 4 heteroatoms selected from nitrogen, oxygen, and sulfur, wherein said 5 to 10 membered heterocycle is substituted with 0-3 R^{6b},
R^{6a}, at each occurrence, is independently selected from is H, Cl, F, Br, I, CN, NO₂, NR¹²R¹³, OR⁵, SR⁴ C(═O)R₄, NR⁵R⁶, S(═O)R⁶, S(═O)₂R⁶,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, and
5 to 10 membered heterocycle containing 1 to 4 heteroatoms selected from nitrogen, oxygen, and sulphur, wherein said 5 to 10 membered heterocycle is substituted with 0-3 R^{1b};
R^{6b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R⁷ is H, C₁-C₄ alkyl, C₂-C₄ alkenyl, or C₃-C₄ alkynyl;
R⁸ is H,
C₁-C₈ alkyl substituted with 0-3 R^{8a},
C₂-C₈ alkenyl substituted with 0-3 R^{8a},
C₂-C₈ alkynyl substituted with 0-3 R^{8a},
C₃-C₁₀ carbocycle substituted with 0-3 R^{8b},
C₁-C₄ sulfonamido substituted with 0-3 R^{8b},
C₆-C₁₀ aryl substituted with 0-3 R^{8b}, or
5 to 10 membered heterocycle containing 1 to 4 heteroatoms selected from nitrogen, oxygen, and sulfur, wherein said 5 to 10 membered heterocycle is substituted with 0-3 R^{8b},
R^{8a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁵, S(═O)₂R⁴,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{8b},
C₁-C₄ sulfonamido substituted with 0-3 R^{8b},
C₆-C₁₀ aryl substituted with 0-3 R^{8b}, and
5 to 10 member heterocycle containing 1 to 4 heteroatoms selected from nitrogen, oxygen, and sulphur, wherein said 5 to 10 member heterocycle is substituted with 0-3 R_{8b};
R^{8b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R¹², at each occurrence, is independently selected from H, C₁-C₆ alkyl, benzyl, phenethyl, (C₁-C₆ alkyl)-C(═O)—, (C₁-C₆ alkyl)-OC(═O)—, (C₁-C₆ alkyl)-S(═O)₂—, and piperdinyl C(═O)—;
R¹³, at each occurrence, is independently selected from H, OH, C₁-C₆ alkyl, benzyl, phenethyl,
(C₁-C₆ alkyl)-C(═O)—, and (C₁-C₆ alkyl)-S(═O)₂—;
alternatively, R¹² and R¹³ together with the nitrogen to which they are attached, may combine to form a 4-7 member ring wherein said 4-7 member ring optionally contains an additional heteroatom selected from O and NH;
R¹⁴, at each occurrence, is independently selected from H, C₁-C₆ alkyl, benzyl, phenethyl, (C₁-C₆ alkyl)-C(═O)—, and (C₁-C₆ alkyl)-S(═O)₂—;
R¹⁵, at each occurrence, is independently selected from H, OH, C₁-C₆ alkyl, benzyl, phenethyl, (C₁-C₆ alkyl)—OC(═O)—,
(C₁-C₆ alkyl)-C(═O)—, and (C₁-C₆ alkyl)-S(═O)2—; and
alternatively, R¹⁴ and R¹⁵, may combine together with the nitrogen to which they are attached, to form a 4-7 member ring,
wherein said 4-7 member ring optionally contains an heteroatom selected from O and NH.

6. A compound of claim 1, according to Formula (VI), 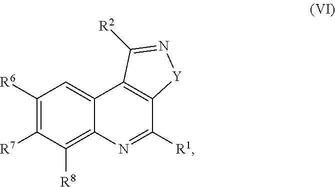
or a stereoisomer or a pharmaceutically acceptable salt form or prodrug thereof, wherein:
Y is CR₃, O, N^{R3},
R¹ is H, O,
C₁-C₈ alkyl substituted with 0-3 R^{1a},
C₂-C₈ alkenyl substituted with 0-3 R^{1a},
C₂-C₈ alkynyl substituted with 0-3 R^{1a},
C₂-C₈ alkoxy substituted with 0-3 R^{1a},
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, or
5 to 10 member heterocycle containing 1 to 4 heteroatoms selected from nitrogen, oxygen, and sulfur, wherein said 5 to 10 member heterocycle is substituted with 0-3 R^{1b},
R^{1a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, and
5 to 10 member heterocycle containing 1 to 4 heteroatoms selected from nitrogen, oxygen, and sulphur, wherein said 5 to 10 member heterocycle is substituted with 0-3 R^{1b};
R^{1b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R² is H,
C₁-C₈ alkyl substituted with 0-3 R^{2a},
C₂-C₈ alkenyl substituted with 0-3 R^{2a},
C₂-C₈ alkynyl substituted with 0-3 R^{2a},
C₃-C₁₀ carbocycle substituted with 0-3 R^{2b},
C₁-C₄ sulfonamido substituted with 0-3 R^{2b},
C₆-C₁₀ aryl substituted with 0-3 R^{2b}, or
5 to 10 member heterocycle containing 1 to 4 heteroatoms selected from nitrogen, oxygen, and sulfur, wherein said 5 to 10 member heterocycle is substituted with 0-3 R^{2b},
R^{2a}, at each occurrence, is independently selected from is H, Cl, F, Br, I, CN, NO₂, NR¹³R¹³, OR⁵, SR⁴, C(═O)R⁴, NR¹⁴R¹⁵, S(═O)R⁶, S(═O)2R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{2b},
C₁-C₄ sulfonamido substituted with 0-3 R^{2b},
C₆-C₁₀ C₆-C₁₀ aryl substituted with 0-3 R^{2b}, and
5 to 10 member heterocycle containing 1 to 4 heteroatoms selected from nitrogen, oxygen, and sulphur, wherein said 5 to 10 member heterocycle is substituted with 0-3 R^{2b};
R^{2b}, at each occurrence, is independently selected from H, OH, Cl, F, Br, I, CN, NO₂, thiazole, NR¹²R¹³, CF₃, acetyl, SCH₃, S(═O)CH₃, S(═O)₂CH₃, H₂N—C(═O)—,
C₁-C₆ alkyl, C₁-C₄ alkoxy, C₁-C₄ haloalkyl, C₁-C₆ cyanoalkyl,
C₁-C₄ haloalkoxy, C₁-C₄ cyanoalkoxy, and C₁-C₄ haloalkyl-S—;
R³ is H, O,
C₁-C₈ alkyl substituted with 0-3 R^{3a},
C₂-C₈ alkenyl substituted with 0-3 R^{3a},
C₂-C₈ alkynyl substituted with 0-3 R^{3a}
C₃-C₁₀ carbocycle substituted with 0-3 R^{3b},
C₁-C₄ sulfonamido substituted with 0-3 R^{3b},
C₆-C₁₀ aryl substituted with 0-3 R^{3b}, or
5 to 10 member heterocycle containing 1 to 4 heteroatoms selected from nitrogen, oxygen, and sulfur, wherein said 5 to 10 member heterocycle is substituted with 0-3 R³,
R^{3a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{3b},
C₁-C₄ sulfonamido substituted with 0-3 R^{3b},
C₆-C₁₀ aryl substituted with 0-3 R^{3b}, and p2 5 to 10 member heterocycle containing 1 to 4 heteroatoms selected from nitrogen, oxygen, and sulphur, wherein said 5 to 10 member heterocycle is substituted with 0-3 R^{3b};
R^{3b}, at each occurrence, is independently selected from H, OH, Cl, F, Br, I, CN, NO₂, NR¹²R¹³, CF₃, acetyl, SCH₃, S(═O)CH₃, S(═O)₂CH₃, H₂N—C(═O)—,
C₁-C₆ alkyl, C₁-C₄ alkoxy, C₁-C₄ haloalkyl, C₁-C₆ cyanoalkyl,
C₁-C₄ haloalkoxy, C₁-C₄ cyanoalkoxy, and C₁-C₄ haloalkyl-S—;
R⁴ is H, phenyl, benzyl, C₁-C₄ alkyl, C₃-C₈ cycloalkyl substituted with 0-3 R^{1b}, or
a 5 to 10 member heterocycle containing 1 to 4 heteroatoms selected from nitrogen, oxygen, and sulphur, wherein said 5 to 10 member heterocycle is substituted with 0-3 R^{1b};
R⁵ is H, phenyl, benzyl, or C₁-C₄ alkyl;
R⁶ is H,
C₁-C₈ alkyl substituted with 0-3 R^{6a},
C₂-C₈ alkenyl substituted with 0-3 R^{6a},
C₂-C₈ alkynyl substituted with 0-3 R^{6a}
C₃-C₁₀ carbocycle substituted with 0-3 R^{6b},
C₁-C₄ sulfonamido substituted with 0-3 R^{6b},
C₆-C₁₀ aryl substituted with 0-3 R^{6a}; arylamine substituted with 0-3 R^{6a},
C₁-C₆ alkyloxy substituted with 0-3 R^{6a}, or
5 to 10 member heterocycle containing 1 to 4 heteroatoms selected from nitrogen, oxygen, and sulfur, wherein said 5 to 10 member heterocycle is substituted with 0-3 R^{6b},
R^{6a}, at each occurrence, is independently selected from is H, Cl, F, Br, I, CN, NO₂, NR¹²R¹³, OR⁵, SR⁴ C(═O)R⁴, NR¹⁴R¹⁵, S(═O)R⁶, S(═O)₂R⁶,
C₁-C₆ ^{alkyl, C}₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, and
5 to 10 member heterocycle containing 1 to 4 heteroatoms selected from nitrogen, oxygen, and sulphur, wherein said 5 to 10 member heterocycle is substituted with 0-3 R^{1b};
R^{6b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R⁷ is H, C₁-C₄ alkyl, C₂-C₄ alkenyl, or C₃-C₄ alkynyl;
R⁸ is H,
C₁-C₈ alkyl substituted with 0-3 R^{8a},
C₂-C₈ alkenyl substituted with 0-3 R^{8a},
C₂-C₈ alkynyl substituted with 0-3 R^{8a}
C₃-C₁₀ carbocycle substituted with 0-3 R^{8b},
C₁-C₄ sulfonamido substituted with 0-3 R^{8b},
C₆-C₁₀ aryl substituted with 0-3 R^{8b}, or
5 to 10 member heterocycle containing 1 to 4 heteroatoms selected from nitrogen, oxygen, and sulfur, wherein said 5 to 10 member heterocycle is substituted with 0-3 R^{8b},
R^{8a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R⁴,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{8b},
C₁-C₄ sulfonamido substituted with 0-3 R^{8b},
C₆-C₁₀ aryl substituted with 0-3 R^{8b}, and
5 to 10 member heterocycle containing 1 to 4 heteroatoms selected from nitrogen, oxygen, and sulphur, wherein said 5 to 10 member heterocycle is substituted with 0-3 R^{8b};
R^{8b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and Cl-C₄ haloalkyl-S—;
R¹², at each occurrence, is independently selected from H, C₁-C₆ alkyl, benzyl, phenethyl, (C₁-C₆ alkyl)-C(═O)—, (C₁-C₆ alkyl)—OC(═O)—, (C₁-C₆ alkyl)-S(═O)₂—, and piperdinyl C(═O)—;
R¹³, at each occurrence, is independently selected from H, OH, C₁-C₆ alkyl, benzyl, phenethyl,
(C₁-C₆ alkyl)-C(═O)—, and (C₁-C₆ alkyl)-S(═O)₂—;
alternatively, R¹² and R¹³ together with the nitrogen to which they are attached, may combine to form a 4-7 member ring wherein said 4-7 member ring optionally contains an additional heteroatom selected from O and NH;
R¹⁴, at each occurrence, is independently selected from H, C₁-C₆ alkyl, benzyl, phenethyl, (C₁-C₆ alkyl)-C(═O)—, and (C₁-C₆ alkyl)-S(═O)₂—;
R¹⁵, at each occurrence, is independently selected from H, OH, C₁-C₆ alkyl, benzyl, phenethyl, (Cᵢ-C₆ alkyl)-OC(═O)—,
(C₁-C₆ alkyl)-C(═O)—, and (C₁-C₆ alkyl)-S(═O)₂—; and
alternatively, R¹⁴ and R¹⁵, may combine together with the nitrogen to which they are attached, to form a 4-7 member ring,
wherein said 4-7 member ring optionally contains an heteroatom selected from O and NH.

7. A compound of claim 1 according to Formula (VII), 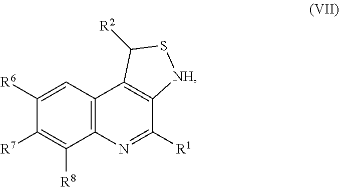
a stereoisomer or a pharmaceutically acceptable salt form or prodrug thereof, wherein:
Y is CR₃, O, NR³,
R¹ is H, O,
C₁-C₈ alkyl substituted with 0-3 R^{1a},
C₂-C₈ alkenyl substituted with 0-3 R^{1a},
C₂-C₈ alkynyl substituted with 0-3 R^{1a},
C₂-C₈ alkoxy substituted with 0-3 R^{1a},
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, or
5 to 10 member heterocycle containing 1 to 4 heteroatoms selected from nitrogen, oxygen, and sulfur, wherein said 5 to 10 member heterocycle is substituted with 0-3 R^{1b},
R^{1a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, and
5 to 10 member heterocycle containing 1 to 4 heteroatoms selected from nitrogen, oxygen, and sulphur, wherein said 5 to 10 member heterocycle is substituted with 0-3 R^{1b};
R^{1b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R² is H,
C₁-C₈ alkyl substituted with 0-3 R^{2a},
C₂-C₈ alkenyl substituted with 0-3 R^{2a},
C₂-C₈ alkynyl substituted with 0-3 R^{2a},
C₃-C₁₀ carbocycle substituted with 0-3 R^{2b},
C₁-C₄ sulfonamido substituted with 0-3 R^{2b},
C₆-C₁₀ aryl substituted with 0-3 R^{2b}, or
5 to 10 member heterocycle containing 1 to 4 heteroatoms selected from nitrogen, oxygen, and sulfur, wherein said 5 to 10 member heterocycle is substituted with 0-3 R^{2b},
R^{2a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{2b},
C₁-C₄ sulfonamido substituted with 0-3 R^{2b},
C₆-C₁₀ aryl substituted with 0-3 R^{2b}, and
5 to 10 member heterocycle containing 1 to 4 heteroatoms selected from nitrogen, oxygen, and sulphur, wherein said 5 to 10 member heterocycle is substituted with 0-3 R^{2b};
R^{2b}, at each occurrence, is independently selected from H, OH, Cl, F, Br, I, CN, NO₂, thiazole, NR₁₂R₁₃, CF₃, acetyl, SCH₃, S(═O)CH₃, S(═O)₂CH₃, H₂N—C(═O)—,
C₁-C₆ alkyl, C₁-C₄ alkoxy, C₁-C₄ haloalkyl, C₁-C₆ cyanoalkyl,
C₁-C₄ haloalkoxy, C₁-C₄ cyanoalkoxy, and C₁-C₄ haloalkyl-S—;
R⁴ is H, phenyl, benzyl, C₁-C₄ alkyl, C₃-C₈ cycloalkyl substituted with 0-3 R^{1b}, or
a 5 to 10 member heterocycle containing 1 to 4 heteroatoms selected from nitrogen, oxygen, and sulphur, wherein said 5 to 10 member heterocycle is substituted with 0-3 R^{1b};
R⁵ is H, phenyl, benzyl, or C₁-C₄ alkyl;
R⁶ is H,
C₁-C₈ alkyl substituted with 0-3 R^{6a},
C₂-C₈ alkenyl substituted with 0-3 R^{6a},
C₂-C₈ alkynyl substituted with 0-3 R^{6a}
C₃-C10 carbocycle substituted with 0-3 R^{6b},
C₁-C₄ sulfonamido substituted with 0-3 R^{6b},
C₆-C₁₀aryl substituted with 0-3 R^{6a}; arylamine substituted with 0-3 R^{6a},
C₁-C₆ alkyloxy substituted with 0-3 R^{6a}, or
5 to 10 member heterocycle containing 1 to 4 heteroatoms selected from nitrogen, oxygen, and sulfur, wherein said 5 to 10 member heterocycle is substituted with 0-3 R^{6b},
R^{6a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R⁶,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, and
5 to 10 member heterocycle containing 1 to 4 heteroatoms selected from nitrogen, oxygen, and sulphur, wherein said 5 to 10 member heterocycle is substituted with 0-3 R^{1b};
R^{6b}, at each occurrence, is independently selected from H, OH, Cl, F, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R⁷ is H, C₁-C₄ alkyl, C₂-C₄ alkenyl, or C₃-C₄ alkynyl;
R⁸ is H,
C₁-C₈ alkyl substituted with 0-3 R^{8a},
C₂-C₈ alkenyl substituted with 0-3 R^{8a},
C₇-C₈ alkynyl substituted with 0-3 R^{8a}
C₃-C₁₀ carbocycle substituted with 0-3 R^{8b},
C₁-C₄ sulfonamido substituted with 0-3 R^{8b},
C₆-C₁₀ aryl substituted with 0-3 R^{8b}, or
5 to 10 member heterocycle containing 1 to 4 heteroatoms selected from nitrogen, oxygen, and sulfur, wherein said 5 to 10 member heterocycle is substituted with 0-3 R^{8b},
R^{8a}, at each occurrence, is independently selected from is H, Cl, F, Br, I, CN, NO₂, NR¹²R¹³, OR⁵, SR⁴, C(═O)R₄, NR¹⁴R¹⁵, S(═O)R⁶, S(═O)₂R⁴,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{8b},
C₁-C₄ sulfonamido substituted with 0-3 R^{8b},
C₆-C₁₀ aryl substituted with 0-3 R^{8b}, and
5 to 10 member heterocycle containing 1 to 4 heteroatoms selected from nitrogen, oxygen, and sulphur, wherein said 5 to 10 member heterocycle is substituted with 0-3 R^{8b};
R^{8b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R¹², at each occurrence, is independently selected from H, C₁-C₆ alkyl, benzyl, phenethyl, (C₁-C₆ alkyl)-C(═O)—, (C₁-C₆ alkyl)—OC(═O)—, (C₁-C₆ alkyl)-S(═O)₂-, and piperdinyl C(═O)—;
R¹³, at each occurrence, is independently selected from H, OH, C₁-C₆ alkyl, benzyl, phenethyl,
(C₁-C₆ alkyl)-C(═O)—, and (C₁-C₆ alkyl)-S(═O)₂—;
alternatively, R¹² and R¹³ together with the nitrogen to which they are attached, may combine to form a 4-7 member ring wherein said 4-7 member ring optionally contains an additional heteroatom selected from O and NH;
R¹⁴, at each occurrence, is independently selected from H, C₁-C₆ alkyl, benzyl, phenethyl, (C₁-C₆ alkyl)-C(═O)—, and (C₁-C₆ alkyl)-S(═O)₂—;
R¹⁵, at each occurrence, is independently selected from H, OH, C₁-C₆ alkyl, benzyl, phenethyl, (C₁-C₆ alkyl)—OC(═O)—,
(C₁-C₆ alkyl)-C(═O)—, and (C₁-C₆ alkyl)-S(═O)₂—; and
alternatively, R¹⁴ and R¹⁵, may combine together with the nitrogen to which they are attached, to form a 4-7 member ring,
wherein said 4-7 member ring optionally contains an heteroatom selected from O and NH.

8. A compound of claim 1 according to Formula (VIII), 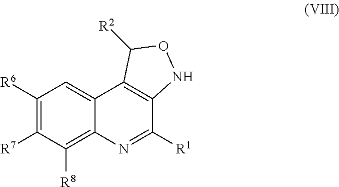
stereoisomer, prodrug, or pharmaceutically acceptable salt forms thereof, wherein:
R¹ is H, O,
C₁-C₈ alkyl substituted with 0-3 R^{1a},
C₂-C₈ alkenyl substituted with 0-3 R^{1a},
C₂-C₈ alkynyl substituted with 0-3 R^{1a},
C₂-C₈ alkoxy substituted with 0-3 R^{1a},
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, or
5 to 10 member heterocycle containing 1 to 4 heteroatoms selected from nitrogen, oxygen, and sulfur, wherein said 5 to 10 member heterocycle is substituted with 0-3 R^{1b},
R^{1a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, and
5 to 10 member heterocycle containing 1 to 4 heteroatoms selected from nitrogen, oxygen, and sulphur, wherein said 5 to 10 member heterocycle is substituted with 0-3 R^{1b};
R^{1b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R² is H,
C₁-C₈ alkyl substituted with 0-3 R^{2a},
C₂-C₈ alkenyl substituted with 0-3 R^{2a},
C₂-C₈ alkynyl substituted with 0-3 R^{2a},
C₃-C₁₀ carbocycle substituted with 0-3 R^{2b},
C₁-C₄ sulfonamido substituted with 0-3 R^{2b},
C₆-C₁₀ aryl substituted with 0-3 R^{2b}, or
5 to 10 member heterocycle containing 1 to 4 heteroatoms selected from nitrogen, oxygen, and sulfur, wherein said 5 to 10 member heterocycle is substituted with 0-3 R^{2b},
R^{2a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R¹⁵,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{2b},
C₁-C₄ sulfonamido substituted with 0-3 R^{2b},
C₆-C₁₀ aryl substituted with 0-3 R^{2b}, and
5 to 10 member heterocycle containing 1 to 4 heteroatoms selected from nitrogen, oxygen, and sulphur, wherein said 5 to 10 member heterocycle is substituted with 0-3 R^{2b};
R^{2b}, at each occurrence, is independently selected from H, OH, Cl, F, Br, I, CN, NO₂, thiazole, NR₁₂R₁₃, CF₃, acetyl, SCH₃, S(═O)CH₃, S(═O)₂CH₃, H₂N—C(═O)—, C₁-C₆ alkyl, C₁-C₄ alkoxy, C₁-C₄ haloalkyl, C₁-C₆ cyanoalkyl, C₁-C₄ haloalkoxy, C₁-C₄ cyanoalkoxy, and C₁-C₄ haloalkyl-S—;
R⁴ is H, phenyl, benzyl, C₁-C₄ alkyl, C₃-C₈ cycloalkyl substituted with 0-3 R^{1b}, or
a 5 to 10 member heterocycle containing 1 to 4 heteroatoms selected from nitrogen, oxygen, and sulphur, wherein said 5 to 10 member heterocycle is substituted with 0-3 R^{1b};
R⁵ is H, phenyl, benzyl, or C₁-C₄ alkyl;
R⁶ is H, C₁-C₈ alkyl substituted with 0-3 R^{6a},
C₂-C₈ alkenyl substituted with 0-3 R^{6a},
C₂-C₈ alkynyl substituted with 0-3 R^{6a}
C₃-C₁₀ carbocycle substituted with 0-3 R^{6b},
C₁-C₄ sulfonamido substituted with 0-3 R^{6b},
C₆-C₁₀aryl substituted with 0-3 R^{6a}; arylamine substituted with 0-3 R^{6a},
C₁-C₆ alkyloxy substituted with 0-3 R^{6a}, or
5 to 10 member heterocycle containing 1 to 4 heteroatoms selected from nitrogen, oxygen, and sulfur, wherein said 5 to 10 member heterocycle is substituted with 0-3 R^{6b},
R^{6a}, at each occurrence, is independently selected from is H, Cl, F, Br, I, CN, NO₂, NR¹²R¹³, OR⁵, SR⁴, C(═O)R⁴, NR¹⁴R¹⁵, S(═O)R⁶, S(═O)₂R⁶,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{1b},
C₁-C₄ sulfonamido substituted with 0-3 R^{1b},
C₆-C₁₀ aryl substituted with 0-3 R^{1b}, and
5 to 10 member heterocycle containing 1 to 4 heteroatoms selected from nitrogen, oxygen, and sulphur, wherein said 5 to 10 member heterocycle is substituted with 0-3 R^{1b};
R^{6b}, at each occurrence, is independently selected from H, OH, Cl, F, Br, I, CN, NO₂, NR¹²R¹³, CF₃, acetyl, SCH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R⁷ is H, C₁-C₄ alkyl, C₂-C₄ alkenyl, or C₃-C₄ alkynyl;
R⁸ is H,
C₁-C₈ alkyl substituted with 0-3 R^{8a},
C₂-C₈ alkenyl substituted with 0-3 R^{8a},
C₂-C₈ alkynyl substituted with 0-3 R^{8a}
C₃-C₁₀ carbocycle substituted with 0-3 R^{8b},
C₁-C₄ sulfonamido substituted with 0-3 R^{8b},
C₆-C₁₀ aryl substituted with 0-3 R^{8b}, or
5 to 10 member heterocycle containing 1 to 4 heteroatoms selected from nitrogen, oxygen, and sulfur, wherein said 5 to 10 member heterocycle is substituted with 0-3 R^{8b},
R^{8a}, at each occurrence, is independently selected from is H, Cl, F, Br, I, CN, NO₂, NR¹²R¹³, OR⁵, SR⁴, C(═O)R₄, NR¹⁴R¹⁵, S(═O)R⁶, S(═O)₂R⁴,
C₁-C₆ alkyl, C₁-C₄ alkoxy, C₁-C₄ haloalkyl,
C₁-C₄ haloalkoxy, C₁-C₄ haloalkyl-S—,
C₃-C₁₀ carbocycle substituted with 0-3 R^{8b},
C₁-C₄ sulfonamido substituted with 0-3 R^{8b},
C₆-C₁₀ aryl substituted with 0-3 R^{8b}, and
5 to 10 member heterocycle containing 1 to 4 heteroatoms selected from nitrogen, oxygen, and sulphur, wherein said 5 to 10 member heterocycle is substituted with 0-3 R^{8b};
R^{8b}, at each occurrence, is independently selected from H, OH, Cl, F, Br, I, CN, NO₂, NR¹²R¹³, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R¹², at each occurrence, is independently selected from H, C₁-C₆ alkyl, benzyl, phenethyl, (C₁-C₆ alkyl)-C(═O)—, (C₁-C₆ alkyl)-OC(═O)—, (C₁-C₆ alkyl)-S(═O)₂—, and piperdinyl C(═O)—;
R¹³, at each occurrence, is independently selected from H, OH, C₁-C₆ alkyl, benzyl, phenethyl,
(C₁-C₆ alkyl)-C(═O)—, and (C₁-C₆ alkyl)-S(═O)₂—;
alternatively, R¹² and R¹³ together with the nitrogen to which they are attached, may combine to form a 4-7 member ring wherein said 4-7 member ring optionally contains an additional heteroatom selected from O and NH;
R¹⁴, at each occurrence, is independently selected from H, C₁-C₆ alkyl, benzyl, phenethyl, (C₁-C₆ alkyl)-C(═O)—, and (C₁-C₆ alkyl)-S(═O)₂—;
R¹⁵, at each occurrence, is independently selected from H, OH, C₁-C₆ alkyl, benzyl, phenethyl, (C₁-C₆ alkyl)—OC(═O)—,
(C₁-C₆ alkyl)-C(═O)—, and (C₁-C₆ alkyl)-S(═O)₂—; and
alternatively, R¹⁴ and R¹⁵, may combine together with the nitrogen to which they are attached, to form a 4-7 member ring,
wherein said 4-7 member ring optionally contains an heteroatom selected from O and NH.

9. A compound of claim 2, according to Formula (II), or a stereoisomer or a pharmaceutically acceptable salt form or prodrug thereof: 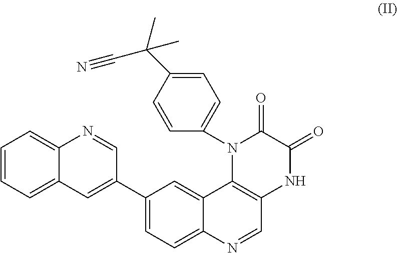
2-(4-(2,3-dioxo-9-(quinolin-3-yl)-3,4-dihydropyrazino[2,3-c]quinolin-1(2H)-yl)phenyl)-2-methylpropanenitrile.

10. A compound of claim 6, according to Formula (VI), or a stereoisomer or a pharmaceutically acceptable salt form or prodrug thereof, selected from: 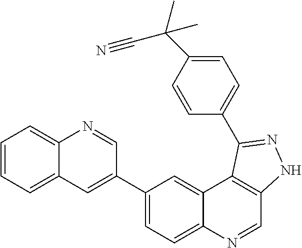
2-methyl-2-(4-(8-(quinolin-3-yl)-3H-pyrazolo[3,4-c]quinolin-1-yl)phenyl)propanenitrile; 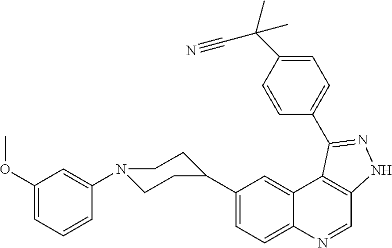
2-(4-(8-(1-(3-methoxyphenyl)piperidin-4-yl)-3H-pyrazolo[3,4-c]quinolin-1-yl)phenyl)-2-methylpropanenitrile; 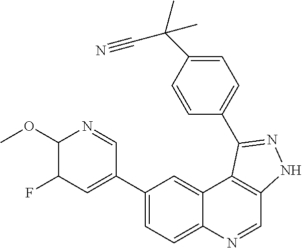
2-(4-(8-(5-fluoro-6-methoxy-5,6-dihydropyridin-3-yl)-3H-pyrazolo[3,4-c]quinolin-1-yl)phenyl)-2-methylpropanenitrile; 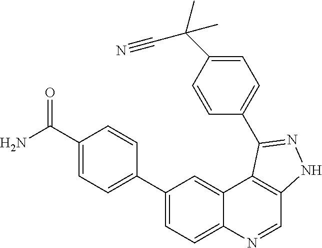
4-(1-(4-(2-cyanopropan-2-yl)phenyl)-3H-pyrazolo[3,4-c]quinolin-8-yl)benzamide; 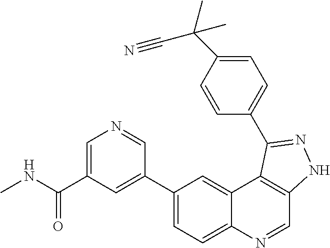
5-(1-(4-(2-cyanopropan-2-yl)phenyl)-3H-pyrazolo[3,4-c]quinolin-8-yl)-N-methylnicotinamide; 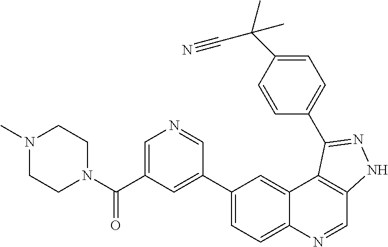
2-methyl-2-(4-(8-(5-(4-methylpiperazine-1-carbonyl)pyridin-3-yl)-3H-pyrazolo[3,4-c]quinolin-1-yl)phenyl)propanenitrile; 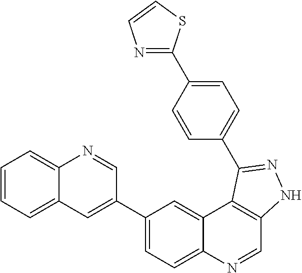
2-(4-(8-(quinolin-3-yl)-3H-pyrazolo[3,4-c]quinolin-1 -yl)phenyl)thiazole; 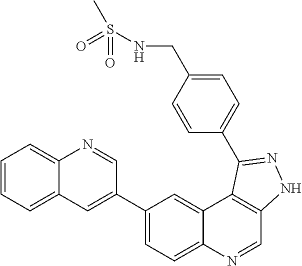
N-(4-(8-(quinolin-3-yl)-3H-pyrazolo[3,4-c]quinolin-1-yl)benzyl)methanesulfonamide; 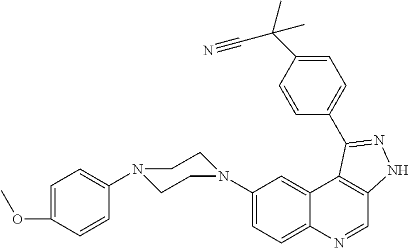
2-(4-(8-(4-(4-methoxyphenyl)piperazin-1-yl)-3H-pyrazolo[3,4-c]quinolin-1-yl)phenyl)-2-methylpropanenitrile; 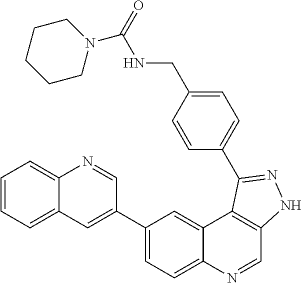
N-(4-(8-(quinolin-3-yl)-3H-pyrazolo[3,4-c]quinolin-1-yl)benzyl)piperidine-1-carboxamide; 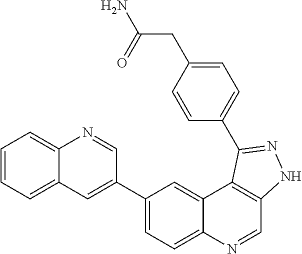
2-(4-(8-(quinolin-3-yl)-3H-pyrazolo[3,4-c]quinolin-1-yl)phenyl)acetamide; 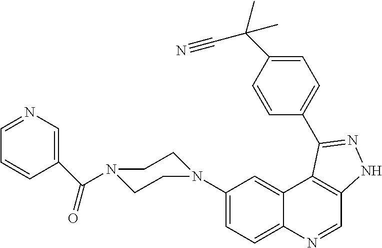
2-methyl-2-(4-(8-(4-nicotinoylpiperazin-1-yl)-3H-pyrazolo[3,4-c]quinolin-1-yl)phenyl)propanenitrile; 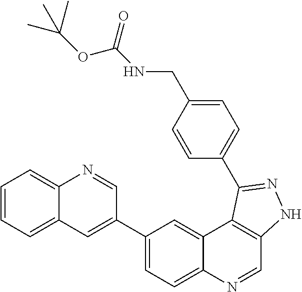
tert-butyl 4-(8-(quinolin-3-yl)-3H-pyrazolo[3,4-c]quinolin-1-yl)benzylcarbamate; 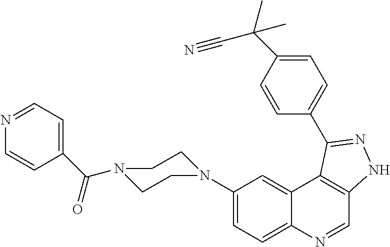
2-(4-(8-(4-isonicotinoylpiperazin-1-yl)-3H-pyrazolo[3,4-c]quinolin-1-yl)phenyl)-2-methylpropanenitrile; 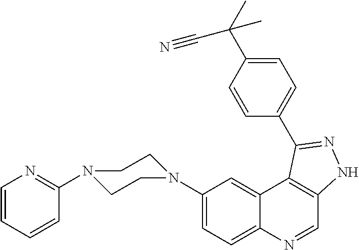
2-methyl-2-(4-(8-(4-(pyridin-2-yl)piperazin-1-yl)-3H-pyrazolo[3,4-c]quinolin-1-yl)phenyl)propanenitrile; 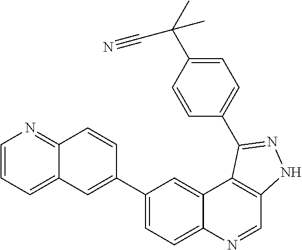
2-methyl-2-(4-(8-(quinolin-6-yl)-3H-pyrazolo[3,4-c]quinolin-1-yl)phenyl)propanenitrile; 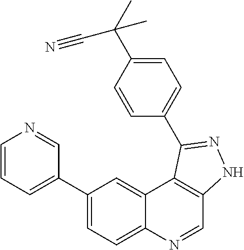
2-methyl-2-(4-(8-(pyridin-3-yl)-3H-pyrazolo[3,4-c]quinolin-1-yl)phenyl)propanenitrile; 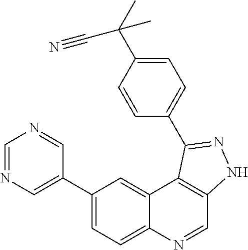
2-methyl-2-(4-(8-(pyrimidin-5-yl)-3H-pyrazolo[3,4-c]quinolin-1-yl)phenyl)propanenitrile; 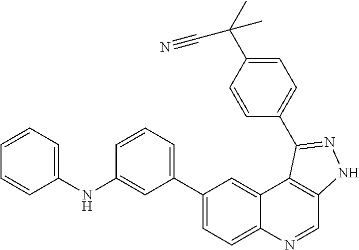
2-methyl-2-(4-(8-(3-(phenylamino)phenyl)-3H-pyrazolo[3,4-c]quinolin-1-yl)phenyl)propanenitrile; 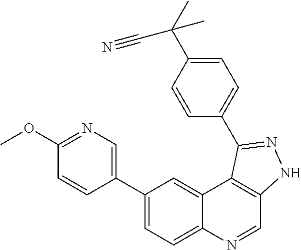
2-(4-(8-(6-methoxypyridin-3-yl)-3H-pyrazolo[3,4-c]quinolin-1-yl)phenyl)-2-methylpropanenitrile; 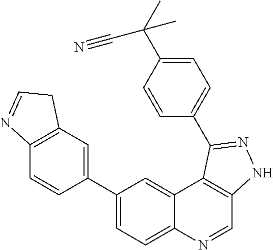
2-(4-(8-(3H-indol-5-yl)-3H-pyrazolo[3,4-c]quinolin-1-yl)phenyl)-2-methylpropanenitrile; 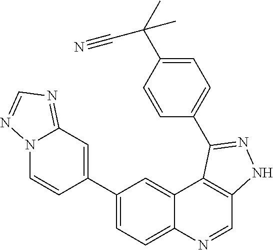
2-(4-(8-(1,3a-dihydro-[1,2,3]triazolo[1,5-a]pyridin-5-yl)-3H-pyrazolo[3,4-c]quinolin-1-yl)phenyl)-2-methylpropanenitrile; 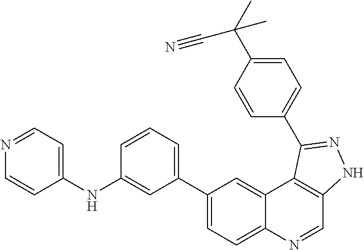
2-methyl-2-(4-(8-(3-(pyridin-4-ylamino)phenyl)-3H-pyrazolo[3,4-c]quinolin-1-yl)phenyl)propanenitrile; 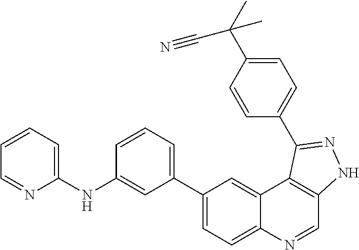
2-methyl-2-(4-(8-(3 -(pyridin-2-ylamino)phenyl)-3H-pyrazolo[3,4-c]quinolin-1-yl)phenyl)propanenitrile; 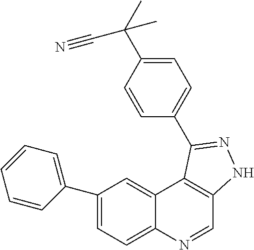
2-methyl-2-(4-(8-phenyl-3H-pyrazolo[3,4-c]quinolin-1-yl)phenyl)propanenitrile; 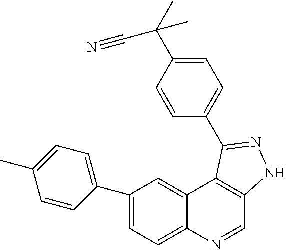
2-methyl-2-(4-(8-p-tolyl-3H-pyrazolo[3,4-c]quinolin-1-yl)phenyl)propanenitrile; 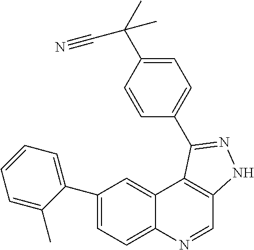
2-methyl-2-(4-(8-o-tolyl-3H-pyrazolo[3,4-c]quinolin-1-yl)phenyl)propanenitrile; 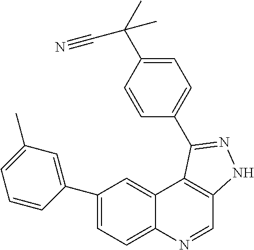
2-methyl-2-(4-(8-m-tolyl-3H-pyrazolo[3,4-c]quinolin-1-yl)phenyl)propanenitrile; 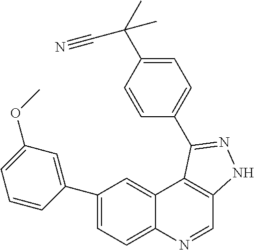
2-(4-(8-(3- ethoxyphenyl)-3H-pyrazolo[3,4-c]quinolin-1-yl)phenyl)-2-methylpropanenitrile; 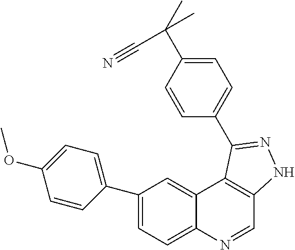
2-(4-(8-(4-methoxyphenyl)-3H-pyrazolo[3,4-c]quinolin-1-yl)phenyl)-2-methylpropanenitrile; 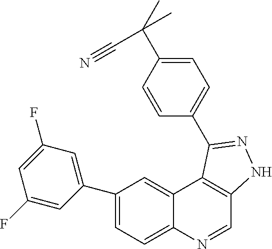
2-(4-(8-(3,5-difluorophenyl)-3H-pyrazolo[3,4-c]quinolin-1-yl)phenyl)-2-methylpropanenitrile; 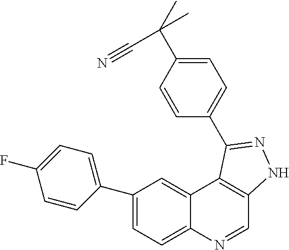
2-(4-(8-(4-fluorophenyl)-3H-pyrazolo[3,4-c]quinolin-1-yl)phenyl)-2-methylpropanenitrile; and 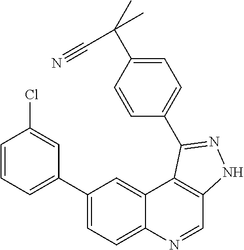
2-(4-(8-(3-chlorophenyl)-3H-pyrazolo[3,4-c]quinolin-1-yl)phenyl)-2-methylpropanenitrile.

11. A compound of claim 6, according to Formula (VI), or a stereoisomer or a pharmaceutically acceptable salt form or prodrug thereof, according to the structure, selected from: 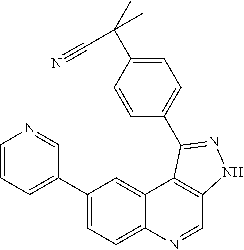
2-methyl-2-(4-(8-(pyridin-3-yl)-3H-pyrazolo[3,4-c]quinolin-1-yl)phenyl)propanenitrile and 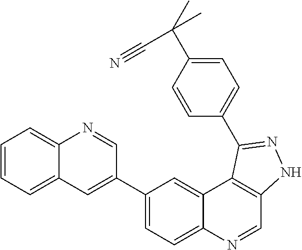
2-methyl-2-(4-(8-(quinolin-3-yl)-3H-pyrazolo[3,4-c]quinolin-1-yl)phenyl)propanenitrile.

12. A compound of claim 2, according to Formula (II), or a stereoisomer or a pharmaceutically acceptable salt form or prodrug thereof, 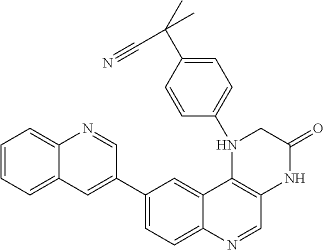
2-methyl-2-(4-(3-oxo-9-(quinolin-3-yl)-3,4-dihydropyrazino[2,3-c]quinolin-1(2H)-yl)phenyl)propanenitrile.

13. A compound of claim 7, according to Formula (VII), or a stereoisomer or a pharmaceutically acceptable salt form or prodrug thereof, 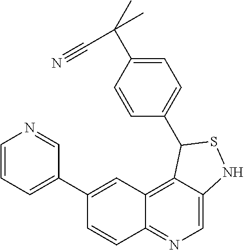
2-methyl-2-(4-(8-(pyridine-3-yl)isothiazolo[3,4-c]quinolin-1-yl)propanenitrile, 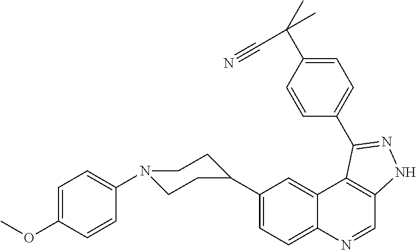
2-(4-(8-(1-(4-methoxyphenyl)piperidin-4-yl)-3H-pyrazolo[3,4-c]quinolin-1-yl)phenyl)-2-methylpropanenitrile, 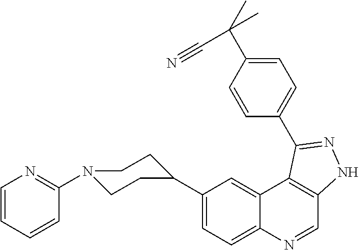
2-methyl-2-(4-(8-(1-(pyridin-2-yl)piperidin-4-yl)-3H-pyrazolo[3,4-c]quinolin-1-yl)phenyl)propanenitrile, 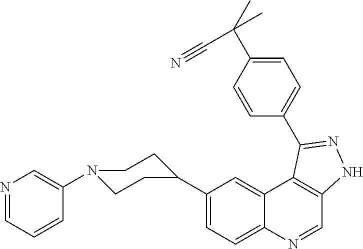
2-methyl-2-(4-(8-(1-(pyridin-3-yl)piperidin-4-yl)-3H-pyrazolo[3,4-c]quinolin-1-yl)phenyl)propanenitrile, 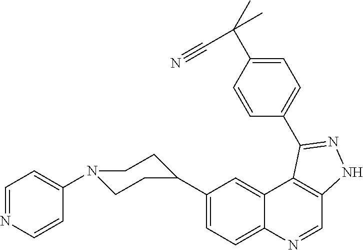
2-methyl-2-(4-(8-(1-(pyridin-4-yl)piperidin-4-yl)-3H-pyrazolo[3,4-c]quinolin-1-yl)phenyl)propanenitrile, 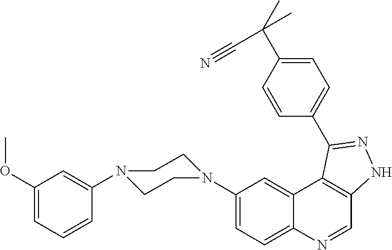
2-(4-(8-(4-(3-methoxyphenyl)piperazin-1-yl)-3H-pyrazolo[3,4-c]quinolin-1-yl)phenyl)-2-methylpropanenitrile, 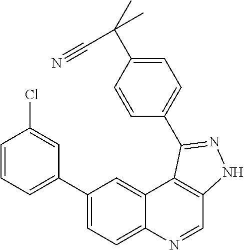
2-(4-(8-(3-chlorophenyl)-3H-pyrazolo[3,4-c]quinolin-1-yl)phenyl)-2-methylpropanenitrile, or 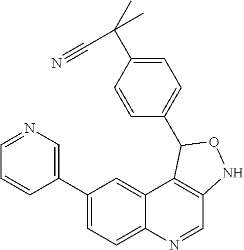
2-methyl-2-(4-(8-(pyridin-3-yl)-1,3-dihydroisoxazolo[3,4-c]quinolin-1-yl)phenyl)propanenitrile.

14. A compound according to claim 1, or a stereoisomer or a pharmaceutically acceptable salt form or prodrug thereof, selected from the group presented in Table A.

15. A compound according to claim 1, or a stereoisomer or a pharmaceutically acceptable salt form or prodrug thereof, selected from the group presented in Table A.

16. A composition comprising a compound, stereoisomer, prodrug or pharmaceutically acceptable salt form thereof, according to any one of the claims 1-15; and a pharmaceutically acceptable diluent or other inert carrier.

17. A method of treating a human or animal disease comprising administering to a subject in need thereof a therapeutically effective amount of a compound according to any one of claims 1-15, a stereoisomer, prodrug, or pharmaceutically acceptable salt form thereof,
wherein the disease comprises cancers, cancer-associated maladies, benign growths, tumor growths, neoplasms, metabolic diseases, inflammatory diseases, allergic diseases, or cardiovascular disease; or
wherein the disease further comprises diseases associated with abnormal activity or activation of PI3-kinase, PI3-kinase subtypes, PI3-kinase mutants or PI3-kinase variants, PI3-kinase-related kinases, PI3-kinase signaling pathway, protein kinases, non-kinases, transcription and protein translation factors, overexpressed or activated oncogenes, or growth factor signal transduction pathways that enhance or cooperate with the PI3-kinase pathway; and/or
wherein the disease further comprises a disease associated with abnormal activity or activation of pathways or enzymes that negatively regulate the PI3-kinase or the PI3-kinase pathway; and/or
wherein the disease further comprises a disease associated with abnormal activity, activation, or overexpression of a protein kinase, a non-kinase, a transcription factor, a protein translation factor, or an oncogene associated with cell growth, proliferation, and/or survival.

18. The method according to claim 17, wherein the oncogene associated with cell growth, proliferation, and/or survival is selected from Ras, c-myc, Cyclin B, cyclin D, or eIF-4E.

19. The method according to claim 17,
wherein the cancers comprise adrenal, bladder, brain, breast, cervical, endometrial, uterine, colon, esophageal, head/neck, kidney, liver, lung, ovarian, pancreatic, prostate, rectal, stomach, thyroid, or vaginal cancer; or
wherein the cancers further comprise leukemia, acute lymphocytic leukemia, chronic myeloid leukemia, and chronic lymphocytic leukemia; multiple myeloma, neuroblastoma, lymphoma, GIST, skin melanoma and Kaposi's sarcoma, sarcoma, or solid tumor; or
wherein the inflammatory, diseases comprise rheumatoid arthritis, osteoarthritis, ankyolsing spondylitis, psoriatic arthritis; psoriasis, systemic lupus erythematosus, glomerulonephritis, scleroderma, general renal failure, inflammatory bowel disease, ulcerative colitis, Crohn's disease, pancreatitis, multiple sclerosis, inflammation due to hyper-responsiveness to cytokine production, chronic obstructive pulmonary, airway or lung disease (COPD, COAD or COLD), acute respiratory distress syndrome (ARDS) and occupation-related diseases comprising aluminosis, anthracosis, asbestosis; chalicosis, ptilosis, siderosis, silicosis, tabacosis or byssinosis; or
wherein the allergic diseases comprise asthma, asthma related, small and large airway hyperactivity, bronanaphylaxis, aspirin-induced asthma, allergic airway inflammation, urticaria, Steven-Johnson syndrome, atopic dermatitis, bolus pemphigoid, or parasite-caused eosinophilia; or
wherein the cardiovascular and metabolic diseases comprise atherosclerosis, acute heart failure, enlargement of the heart, myocardial infarction and reprofusion injury, type-2 diabetes, syndrome X, and obesity; or
wherein the abnormal activity, activation, or overexpression involves one or more kinases selected from the protein kinases ABL1, ABL2, ALK4, ARK5, AUR A, AXL, BLK, BMX, BRK, BTK, CAMKK2, CDK1, CDK2, CDK3, CDK5, CDK7, CK1δ, CK1ε, CK2α, CK2α2, CLK1, CLK2, CLK3, CLK4, c-MER, c-Src, DYRKIA, DYRK1B,DYRK2, DYRK3, EGFR, EPHA7, FER, FGR, FLT3, FLT4, FMS, FYN, GCK, GSK3α, GSK3β, HCK, HGK, HIPK2, HIPK3, HIPK4, IRAK1, IRAK4, ITK, KDR/VEGFR2, KIT, LCK, LOK, LYN, MELK, MLCK2, MLK1, MNK1, MNK2, MST1, MST2, mTOR, MUSK, NEK1, NEK3, PDGFRα, PDGFRβ, PIM-1, PKCδ (delta), PKCμ (mu), PKCν (nu), PKD2, RET, RIPK2, ROS, RSK1, RSK2, RSK3, RSK4, STK33, TAK1, TAOK1, TAOK3, TRKA, TRKB, TRKC, TTK, TXK,TYK2, YES, ZAK, or ZAP70, or mutant, mutationally activated, or variant forms thereof; or
wherein the enzymes that negatively regulate the PI3-kinase or the PI3-kinase pathway comprise PTEN or a mutant or variant form thereof.

20. The method according to the claim 17 or 19, further comprising administering the compound or composition alone or in combination with one or more additional therapeutics, chemotherapeutic drugs, antiproliferative agents, anti-inflammatory agents, agents for treating asthma, immunosuppressive agents, immunomodulatory agents, cardiovascular disease treatment agents, diabetes treatment agents, blood disorder treatment agents, or in combination with one or more non-PI3-kinase inhibitors.

21. The method according to claim 20, wherein additional chemotherapeutic agents or other antiproliferative agents may be co-administered, administered at the same or a different time, or combined to treat the disease.

22. The method according to claim 20, wherein one or more chemotherapeutic drugs comprise alkylating drugs, cyclophosphamide, melphalan, mechlorethamine, chlorambucil, Ifosfamide; antimetabolites; or methotrexate; wherein the one or more chemotherapeutic drugs comprise purine antagonists or pyrimidine antagonists, 6-mercaptopurine, 5-fluorouracil, fluorouracil, cytarabile, gemcitabine; wherein the one or more chemotherapeutic drugs comprise spindle poisons, vinblastine, vincristine, vinorelbine, or paclitaxel; wherein the one or more chemotherapeutic drugs comprise podophyllotoxins, etoposide, irinotecan, topotecan; wherein the one or more chemotherapeutic drugs comprise antibiotics, doxorubicin, bleomycin, mitomycin, adriamycin, dexamethasone; wherein the one or more chemotherapeutic drugs comprise nitrosoureas, Carmustine, Lomustine; wherein the one or more chemotherapeutic drugs comprise inorganic ions, cisplatin, carboplatin; wherein the one or more chemotherapeutic drugs comprise enzymes, asparaginase; wherein the one or more chemotherapeutic drugs comprise biologic response modifiers, interleukins, tumor suppressor factors, interleukins, tumor necrosis factor (TNF), hormones, Tamoxifen, Leuprolide, Flutamide, or Megestrol; wherein the one or more chemotherapeutic drugs comprise small molecule inhibitor drugs, Gleevec®, Sutent®; cyclophosphamide, Taxol, or platinum derivatives.

23. The method according to claim 20, wherein one or more additional therapeutics comprise anti-inflammatory agents, non-steroidal anti-inflammatory drugs (NSAIDs), corticosteroids, TNF blockers or inhibitors, IL-RA, azathioprine, cyclophosphamide, sulfasalazine; wherein the one or more additional agents comprise treatments for allegeric diseases, agents for treating asthma, albuterol, Singulair®; wherein the one or more additional comprise agents for treating multiple sclerosis, β-interferon, Avonex®, Rebif®, Copaxone®, mitoxantrone; wherein the one or more additional agents comprise immunosuppressive and immunomodulatory agents, cyclosporin, tacrolimus, rapamycin, mycophenolate mofetil, interferons, corticosteroids, cyclophosphamide, azathioprine, or sulfasalazine; wherein the one or more additional agents comprise cardiovascular disease treatment agents, ACE inhibitors, beta-blockers, diuretics, nitrates, calcium channel blockers, statins; wherein the one or more additional agents comprise diabetes treatment agents, insulin, glitazones, sulfonyl ureas; wherein the one or more additional agents include blood disorder treatment agents, corticosteroids, or anti-leukemia agents.

24. A method of treating, reducing the severity of, inhibiting the growth of, eliminating, or preventing a tumor or cancer associated with activation, aberrant expression, aberrant activity, or overexpression of PI3K in a subject, comprising administering to the subject an effective amount of the compound of any one of claims 1-15 or the composition of claim 16, so as to treat, reduce the severity of, inhibit the growth of, eliminate, or prevent the tumor or cancer;
wherein the tumor or cancer comprises adrenal, bladder, brain, breast, cervical, endometrial, uterine, colon, esophageal, head/neck, kidney, liver, lung, ovarian, pancreatic, prostate, rectal, stomach, thyroid, or vaginal tumors or cancers; or further, wherein the cancer is one or more of leukemia, acute lymphocytic leukemia, chronic myeloid leukemia, chronic lymphocytic leukemia; multiple myeloma, neuroblastoma, lymphoma, GIST, skin melanoma, Kaposi's sarcoma, sarcoma, or solid tumor.

25. A method of treating, reducing the severity of, inhibiting the growth of, eliminating, or preventing a tumor or cancer associated with activation, aberrant expression, aberrant activity, or overexpression of PI3K in a subject, comprising administering to the subject an effective amount of the compound of any one of claims 1-15 or the composition of claim 16 wherein the compound or composition is administered alone or in combination with one or more additional therapeutics, chemotherapeutic drugs, antiproliferative agents, anti-inflammatory agents, immunosuppressive agents, immunomodulatory agents, or one or more non-PI3-kinase inhibitors.

26. A method of treating, reducing the severity of, inhibiting the growth of, eliminating, or preventing a tumor or cancer associated with activation, aberrant expression, aberrant activity, or overexpression of PI3K in a subject, comprising administering to the subject an effective amount of the compound of any one of claims 1-15 or the composition of claim 16, wherein the tumor or cancer is associated with activation, aberrant expression, aberrant activity, or overexpression of PI3Kα(p110α), PI3β(p110β), PI3Kγ(p110γ), PI3Kδ(p110δ), or a mutant or variant form thereof.

27. The method of claim 26, wherein the tumor or cancer is associated with activation, aberrant expression, aberrant activity, or overexpression of PI3Kα(p110α), or a mutant or variant form thereof.

28. The method of claim 26, wherein the tumor or cancer is associated with activation, aberrant expression, aberrant activity, or overexpression of one or more of PI3Kα(E545K) or PI3Kα(H1047R).

29. A method of treating, reducing the severity of, inhibiting, eliminating, or preventing a disease or condition associated with activation, aberrant expression, aberrant activity, or overexpression of P13K in a subject, comprising administering to the subject an effective amount of the compound of any one of claims 1-15 or the composition of claim 16, so as to treat, reduce the severity of, inhibit, eliminate, or prevent the disease or condition; wherein the disease or condition is one or more of inflammatory diseases, allergic diseases, metabolic diseases, cardiovascular disease, or a disease or condition associated therewith; wherein the disease or condition further comprises diseases or conditions associated with abnormal activity or activation of PI3-kinase, PI3-kinase subtypes, PI3-kinase mutants or PI3-kinase variants, PI3-kinase-related kinases, PI3-kinase signaling pathway, protein kinases, non-kinases, transcription and protein translation factors, overexpressed or activated oncogenes, or growth factor signal transduction pathways that enhance or cooperate with the PI3-kinase pathway; wherein the disease or condition further comprises a disease or condition associated with abnormal activity or activation of pathways or enzymes that negatively regulate the PI3-kinase or the PI3-kinase pathway; wherein the disease or condition further comprises a disease or condition associated with abnormal activity, activation, expression, or overexpression of a protein kinase associated with cell growth, proliferation, and/or survival.

30. A method of treating, reducing the severity of, inhibiting, eliminating, or preventing a disease or condition, comprising administering to the subject an effective amount of the compound of any one of claims 1-15 or the composition of claim 16, wherein the disease or condition is or involves:
(i) an inflammatory, disease which comprises rheumatoid arthritis, osteoarthritis, ankyolsing spondylitis, psoriatic arthritis; psoriasis, systemic lupus erythematosus, glomerulonephritis, scleroderma, general renal failure, inflammatory bowel disease, ulcerative colitis, Crohn's disease, pancreatitis, multiple sclerosis, inflammation due to hyper-responsiveness to cytokine production, chronic obstructive pulmonary, airway or lung disease (COPD, COAD, or COLD), acute respiratory distress syndrome (ARDS) and occupation-related diseases comprising aluminosis, anthracosis, asbestosis; chalicosis, ptilosis, siderosis, silicosis, tabacosis, or byssinosis;
(ii) an allergic disease which comprises asthma, asthma related, small and large airway hyperactivity, bronanaphylaxis, aspirin-induced asthma, allergic airway inflammation, urticaria, Steven-Johnson syndrome, atopic dermatitis, bolus pemphigoid, and parasite-caused eosinophilia;
(iii) a cardiovascular or metabolic disease which comprises atherosclerosis, acute heart failure, enlargement of the heart, myocardial infarction and reprofusion injury, type-2 diabetes, syndrome X, or obesity; and
(iv) abnormal activity, activation, or overexpression of one or more kinases selected from kinases ABL1, ABL2, ALK4, ARK5, AUR A, AXL, BLK, BMX, BRK, BTK, CAMKK2, CDK1, CDK2, CDK3, CDK5, CDK7, CK1δ, CK1ε, CK2α, CK2α2, CLK1, CLK2, CLK3, CLK4, c-MER, c-Src, DYRK1A, DYRK1B,DYRK2, DYRK3, EGFR, EPHA7, FER, FGR, FLT3, FLT4, FMS, FYN, GCK, GSK3α, GSK3β, HCK, HGK, HIPK2, HIPK3, HIPK4, IRAK1, IRAK4, ITK, KDR/VEGFR2, KIT, LCK, LOK, LYN, MELK, MLCK2, MLK1, MNK1, MNK2, MST1, MST2, mTOR, MUSK, NEK1, NEK3, PDGFRα, PDGFRβ, PIM-1, PKCδ (delta), PKCμ (mu), PKCν (nu), PKD2, RET, RIPK2, ROS, RSK1, RSK2, RSK3, RSK4, STK33, TAK1, TAOK1, TAOK3, TRKA, TRKB, TRKC, TTK, TXK,TYK2, YES, ZAK, ZAP70, or mutant, mutationally activated, or variant forms thereof.

31. The method of claim 30, wherein the compound or composition is administered alone or in combination with one or more additional therapeutics, chemotherapeutic drugs, antiproliferative agents, anti-inflammatory agents, agents for treating asthma, anti-allergic agents, immunosuppressive agents, immunomodulatory agents, cardiovascular disease treatment kinase inhibitors.

32. The method of claim 30, wherein the compound is effective for treating, reducing the severity of, inhibiting the growth of, eliminating, or preventing a tumor or cancer associated with activation, aberrant expression, aberrant activity, or overexpression of PI3K in a subject.

33. The method of of claim 30, wherein the disease or condition is associated with activation, aberrant expression, aberrant activity, or overexpression of PI3Kα(p110α), PI3Kβ(p110β), PI3Kγ(p110γ), PI3Kδ(p110δ), or a mutant or variant form thereof.

34. The method of claim 33, wherein the disease or condition is associated with activation, aberrant expression, aberrant activity, or overexpression of PI3Kα(p110α), or a mutant or variant form thereof.

35. The method of claim 33, wherein the the disease or condition is associated with activation, aberrant expression, aberrant activity, or overexpression of one or more of PI3Kα (E545K) or PI3Kα(H1047R).

36. A method of treating, reducing the severity of, inhibiting the growth of, eliminating, or preventing a tumor, cancer, or disease associated with activation, aberrant expression, aberrant activity, or overexpression of one or more of ABL1, ABL2, ALK4, ARK5, AUR A, AXL, BLK, BMX, BRK, BTK, CAMKK2, CDK1, CDK2, CDK3, CDK5, CDK7, CK1δ, CK1ε, CK2α, CK2α2, CLK1, CLK2, CLK3, CLK4, c-MER, c-Src, DYRK1A, DYRK1B,DYRK2, DYRK3, EGFR, EPHA7, FER, FGR, FLT3, FLT4, FMS, FYN, GCK, GSK3α, GSK3β, HCK, HGK, HIPK2, HIPK3, HIPK4, IRAK1, IRAK4, ITK, KDR/VEGFR2, KIT, LCK, LOK, LYN, MELK, MLCK2, MLK1, MNK1, MNK2, MST1, MST2, mTOR, MUSK, NEK1, NEK3, PDGFRα, PDGFRβ, PIM-1, PKCδ (delta), PKCμ (mu), PKCν (nu), PKD2, RET, RIPK2, ROS, RSK1, RSK2, RSK3, RSK4, STK33, TAK1, TAOK1, TAOK3, TRKA, TRKB, TRKC, TTK, TXK,TYK2, YES, ZAK, ZAP70 kinases, or mutant, mutationally activated, or variant forms thereof, in a subject, comprising administering to the subject an effective amount of the compound of any one of claims 1-15, or the composition of claim 16, so as to treat, reduce the severity of, inhibit the growth of, eliminate, or prevent the tumor, cancer, or disease.

37. The method according to claim 36, wherein the tumor or cancer is one or more of adrenal, bladder, brain, breast, cervical, endometrial, uterine, colon, esophageal, head/neck, kidney, liver, lung, ovarian, pancreatic, prostate, rectal, stomach, thyroid, vaginal tumors or cancers, leukemia, acute lymphocytic leukemia, chronic myeloid leukemia, chronic lymphocytic leukemia; multiple myeloma, neuroblastoma, lymphoma, GIST, skin melanoma, Kaposi's sarcoma, sarcoma, solid tumor, breast tumor or cancer, colorectal tumor or cancer, lung tumor or cancer, brain tumor or cancer, or ovarian tumor or cancer.

38. The method according to claim 36, wherein the compound or composition is administered alone or in combination with one or more additional therapeutics, chemotherapeutic drugs, antiproliferative agents, anti-inflammatory agents, agents for treating asthma, immunosuppressive agents, immunomodulatory agents, cardiovascular disease treatment agents, diabetes treatment agents, blood disorder treatment agents, or one or more non-PI3-kinase inhibitors.

39. The method of claim 38, wherein the tumor or cancer is associated with activation, aberrant expression, or overexpression of PI3Kα, or a mutant or variant form thereof.

40. A method of treating, reducing the severity of, inhibiting the growth of, eliminating, or preventing a tumor, cancer, disease or condition associated with activation, aberrant expression, aberrant activity, or overexpression of both PI3K and one or more of ABL1, ABL2, ALK4, ARK5, AUR A, AXL, BLK, BMX, BRK, BTK, CAMKK2, CDK1, CDK2, CDK3, CDK5, CDK7, CK1δ, CK1ε, CK2α, CK2α2, CLK1, CLK2, CLK3, CLK4, c-MER, c-Src, DYRK1A, DYRK1B,DYRK2, DYRK3, EGFR, EPHA7, FER, FGR, FLT3, FLT4, FMS, FYN, GCK, GSK3α, GSK3β, FICK, HGK, HIPK2, HIPK3, HIPK4, IRAK1, IRAK4, ITK, KDR/VEGFR2, KIT, LCK, LOK, LYN, MELK, MLCK2, MLK1, MNK1, MNK2, MST1, MST2, mTOR, MUSK, NEK1, NEK3, PDGFRα, PDGFRβ, PIM-1, PKCδ (delta), PKCμ (mu), PKCν (nu), PKD2, RET, RIPK2, ROS, RSK1, RSK2, RSK3, RSK4, STK33, TAK1, TAOK1, TAOK3, TRKA, TRKB, TRKC, TTK, TXK,TYK2, YES, ZAK, ZAP70 kinases, or mutant, mutationally activated, or variant forms thereof, in a subject, comprising administering to the subject an effective amount of the compound of any one of claims 1-15 or the composition of claim 16, so as to treat, reduce the severity of, inhibit the growth of, eliminate, or prevent the tumor or cancer.

41. The method according to claim 40, wherein the tumor or cancer is one or more of adrenal, bladder, brain, breast, cervical, endometrial, uterine, colon, esophageal, head/neck, kidney, liver, lung, ovarian, pancreatic, prostate, rectal, stomach, thyroid, vaginal tumors or cancers, leukemia, acute lymphocytic leukemia, chronic myeloid leukemia, chronic lymphocytic leukemia; multiple myeloma, neuroblastoma, lymphoma, GIST, skin melanoma, Kaposi's sarcoma, sarcoma, solid tumor, breast tumor or cancer, colorectal tumor or cancer, lung tumor or cancer, brain tumor or cancer, or ovarian tumor or cancer.

42. The method according to claim 40, wherein the compound or composition is administered alone or in combination with one or more additional therapeutics, chemotherapeutic drugs, antiproliferative agents, anti-inflammatory agents, agents for treating asthma, immunosuppressive agents, immunomodulatory agents, cardiovascular disease treatment agents, diabetes treatment agents, blood disorder treatment agents, or one or more non-PI3-kinase inhibitors.

43. The method of claim 40, wherein the tumor or cancer is associated with activation, aberrant expression, aberrant activity, or overexpression of PI3Kα, or a mutant or variant form thereof.

44. A method of treating, reducing the severity of, inhibiting the growth of, eliminating, or preventing a tumor, cancer, disease or condition associated with activation, aberrant expression, aberrant activity, or overexpression of FLT3, FLT3(D835Y), TRKc, MELK, MNK, PDGFRα(D816V), PDGFRα(D842V), PDGFRβ, GSK3α/β, c-MER, CLK1, CLK4, DYRK2, CK2α2, BLK, CDK1, CDK2, LCK, GCK, HCK, IRAK1, IRAK4, ITK, LYN, RIPK2, or PIM-1 in a subject, comprising administering to the subject an effective amount of the compound of any one of claims 1-15 or the composition of claim 16, so as to treat, reduce the severity of, inhibit the growth of, eliminate, or prevent the tumor or cancer.

45. A method of inhibiting PI3K in a cell or biological sample in which PI3K activity is associated with abnormal cell growth, proliferation, survival, or tumorigenesis, comprising contacting the cell or sample with a compound according to any one of claims 1-15 or a composition according to claim 16, in an amount effective for inhibiting the PI3K activity in the cell or sample.

46. The method of claim 45, wherein the cell or biological sample is present in a subject and the compound or composition is administered in an amount effective to inhibit the activity of PI3K in the cell or sample.

47. The method of claim 46, wherein the PI3K activity inhibited in the cell or sample is PI3Kα activity.

48. The method of claim 45, wherein the compound further inhibits the activity of one or more of both PI3K and one or more of ABL1, ABL2, ALK4, ARK5, AUR A, AXL, BLK, BMX, BRK, BTK, CAMKK2, CDK1, CDK2, CDK3, CDK5, CDK7, CK1δ, CK1ε, CK2α, CK2α2, CLK1, CLK2, CLK3, CLK4, c-MER, c-Src, DYRK1A, DYRK1B,DYRK2, DYRK3, EGFR, EPHA7, FER, FGR, FLT3, FLT4, FMS, FYN, GCK, GSK3α, GSK3β, HCK, HGK, HIPK2, HIPK3, HIPK4, IRAK1, IRAK4, ITK, KDRNEGFR2, KIT, LCK, LOK, LYN, MELK, MLCK2, MLK1, MNK1, MNK2, MST1, MST2, mTOR, MUSK, NEK1, NEK3, PDGFRα, PDGFRβ, PIM-1, PKCδ (delta), PKCμ (mu), PKCν (nu), PKD2, RET, RIPK2, ROS, RSK1, RSK2, RSK3, RSK4, STK33, TAK1, TAOK1, TAOK3, TRKA, TRKB, TRKC, TTK, TXK,TYK2, YES, ZAK, ZAP70 kinases, or mutant, mutationally activated, or variant forms thereof, in the cell or sample.

49. The method of claim 48, wherein TRKc, MELK, PIM-1, or MNK activity is inhibited in the cell or sample.

50. The method of claim 48, wherein both PI3K activity and TRKc, MELK, PIM-1, or MNK activity are inhibited in the cell or sample.

51. A method of synthesizing a compound according to any one of claims 1-15, comprising of the steps of:
(a) obtaining a quinoline precursor wherein the quinoline precursor comprises impure source of quinoline, purified quionline and a derivative of quinoline;
(b) modifying the quinoline precursor to yield a quinoline derivative with a leaving group bonded independently to positions 6 and 4 of the quinoline derivative;
(c) chemically substituting the leaving group of the quinoline derivative with an additional group;
(d) modifying the quinoline derivative comprising the additional group to yield a heterocyclicquinoline compound; and
(e) purifying the heterocyclicquinoline compound.

52. The method of claim 51, wherein, when the leaving group of step (b) comprises halogen, the quinoline further comprises a nitrogen atom bonded to position 3 of the quinoline precursor.

53. The method of claim 51, wherein, in step (c), the additional group comprises palladium, hydrogen, boron, organoboronic acid, halide, or trifilate.

54. A method of inducing apoptosis of a tumor or cancer cell, comprising contacting the tumor or cancer cell with a compound according to any one of claims 1-15 or the composition according to claim 16, in an amount effective to induce apoptosis of the tumor or cancer cell.

55. The method of claim 54, wherein the tumor or cancer cell is present in a subject and the compound is administered to the subject.

56. A method of inducing caspase activity in a tumor or cancer cell harboring one or more mutations that confer resistance to a PI3K inhibitor resulting in apoptosis of the tumor or cancer cell, comprising contacting the tumor or cancer cell with a compound according to any one of claims 1-15 or a composition according to claim 16, in an amount effective to induce caspase activity in and apoptosis of the tumor or cancer cell.

57. The method of claim 56, wherein the tumor or cancer cell harbors at least one mutation in one or more of Ras or Src.

58. The method of claim 56, wherein the tumor or cancer cell is present in a subject and the compound is administered to the subject.

59. A method of inducing caspase activity in a tumor or cancer cell comprising overexpression of a gene or protein that confers resistance to a PI3K inhibitor resulting in apoptosis of the tumor or cancer cell, comprising contacting the tumor or cancer cell with a compound according to any one of claims 1-15 or a composition according to claim 16, in an amount effective to induce caspase activity in and apoptosis of the tumor or cancer cell.

60. The method of claim 61, wherein the tumor or cancer cell comprises overexpression of Myc or cyclin B.

62. The method of claim 59, wherein the tumor or cancer cell is present in a subject and the compound is administered to the subject.

63. A method of inducing cytotoxicity in a tumor or cancer cell by blocking translation of one or more proteins comprising a signal transduction pathway other than a pathway involving AKT-mTOR, comprising contacting the tumor or cancer cell with a compound according to any one of claims 1-15 or a composition according to claim 16, in an amount effective to block translation of proteins comprising a signal transduction pathway other than the pathway involving AKT-mTOR.

64. The method of claim 63, wherein the one or more proteins is selected from MNK, eIF4E, MAPK, RSK, or a combination thereof.

65. The method of claim 63, wherein the tumor or cancer cell is present in a subject and the compound is administered to the subject.
